SEQUENCE LISTING (78 sequences)

SEQ ID NO: 1 - protein, 556 aa, Homo sapiens
  source 1..556: note = GPC3
QPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSPLKLLTSMAISVVCFFFLVH

SEQ ID NO: 2 - protein, 556 aa, Macaca fascicularis
  source 1..556: note = GPC3
QPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSVPKGRVLDKNLDEEGFESGDCGDDEDECIGGSGDGMMKVKNQLRFLAELAYDLDVDDVPGNNQQATPKDNEISTFHNLGNVHSPLKLLTSMAISVVCFFFLVH

SEQ ID NO: 3 - protein, 8 aa, synthetic construct
  source 1..8: note = IMGT HCDR1
GYTFTDYE

SEQ ID NO: 4 - protein, 8 aa, synthetic construct
  source 1..8: note = IMGT HCDR2
IDPETGDT

SEQ ID NO: 5 - protein, 8 aa, synthetic construct
  source 1..8: note = IMGT HCDR3
TRYFSFAY

SEQ ID NO: 6 - protein, 5 aa, synthetic construct
  source 1..5: note = Kabat HCDR1
DYEMH

SEQ ID NO: 7 - protein, 17 aa, synthetic construct
  source 1..17: note = Kabat HCDR2
AIDPETGDTAYNQKFKG

SEQ ID NO: 8 - protein, 6 aa, synthetic construct
  source 1..6: note = Kabat, Chothia, AbM HCDR3
YFSFAY

SEQ ID NO: 9 - protein, 7 aa, synthetic construct
  source 1..7: note = Chothia HCDR1
GYTFTDY

SEQ ID NO: 10 - protein, 6 aa, synthetic construct
  source 1..6: note = Chothia HCDR2
DPETGD

SEQ ID NO: 11 - protein, 10 aa, synthetic construct
  source 1..10: note = AbM HCDR1
GYTFTDYEMH

SEQ ID NO: 12 - protein, 10 aa, synthetic construct
  source 1..10: note = AbM HCDR2
AIDPETGDTA

SEQ ID NO: 13 - protein, 6 aa, synthetic construct
  source 1..6: note = Contact HCDR1
TDYEMH

SEQ ID NO: 14 - protein, 13 aa, synthetic construct
  source 1..13: note = Contact HCDR2
WIGAIDPETGDTA

SEQ ID NO: 15 - protein, 7 aa, synthetic construct
  source 1..7: note = Contact HCDR3
TRYFSFA

SEQ ID NO: 16 - protein, 11 aa, synthetic construct
  source 1..11: note = IMGT LCDR1
QSLRHSNGNTY

SEQ ID NO: 17 - protein, 9 aa, synthetic construct
  source 1..9: note = IMGT, Kabat, Chothia, AbM LCDR3
YQSKHVPYT

SEQ ID NO: 18 - protein, 16 aa, synthetic construct
  source 1..16: note = Kabat, Chothia, AbM LCDR1
RSSQSLRHSNGNTYLQ

SEQ ID NO: 19 - protein, 7 aa, synthetic construct
  source 1..7: note = Kabat, Chothia, AbM LCDR2
KVSNRFS

SEQ ID NO: 20 - protein, 12 aa, synthetic construct
  source 1..12: note = Contact LCDR1
RHSNGNTYLQWY

SEQ ID NO: 21 - protein, 10 aa, synthetic construct
  source 1..10: note = Contact LCDR2
LLIYKVSNRF

SEQ ID NO: 22 - protein, 8 aa, synthetic construct
  source 1..8: note = Contact LCDR3
YQSKHVPY

SEQ ID NO: 23 - protein, 217 aa, Homo sapiens
  source 1..217: note = IgG1 Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 329 aa, synthetic construct
  source 1..329: note = CH1-hinge-CH2-CH3 (IGHG1*01)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 329 aa, synthetic construct
  source 1..329: note = CH1-hinge-CH2-CH3 (IGHG1*03)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 107 aa, synthetic construct [sequence omitted]

SEQ ID NO: 27 - protein, 115 aa, synthetic construct
  source 1..115: note = CL_#27282 M3-H1L1 VH
EVQLVQSGAEVKKPGSSVKVSCKASGYTFTDYEMHWVRQAPGQGLEWIGAIDPETGDTAYNQKFKGRATLTADKSTSTAYMELSSLRSEDTAVYYCTRYFSFAYWGQGTLVTVSS

SEQ ID NO: 28 - protein, 112 aa, synthetic construct
  source 1..112: note = CL_#26960 M3-H1L1 VL
DVQMTQSPSSVSASVGDRVTITCRSSQSLRHSNGNTYLQWYQQKPGKAPKLLIYKVSNRFSGVPSRFSGSGSGTDFTLTISSLQPEDFATYFCYQSKHVPYTFGQGTKVEIK

SEQ ID NO: 29 - protein, 115 aa, synthetic construct
  source 1..115: note = CL_#28294 M3-H18L6 with C-term K, VH
QVQLVQSGAEVKKPGSSVKVSCKASGYTFTDYEMHWVKQAPGHGLEWIGAIDPETGDTAYNQKFKGKATLTADTSTSTAYMELSSLRSEDTAVYYCTRYFSFAYWGQGTLVTVSS

SEQ ID NO: 30 - protein, 112 aa, synthetic construct
  source 1..112: note = CL_#28285 M3-H18L6 VL
EIVLTQSPATLSLSPGERATISCRSSQSLRHSNGNTYLQWYQQKPGQAPRLLIYKVSNRFSGVPARFSGSGSGTDFTLTISSLEPEDFAVYFCYQSKHVPYTFGGGTKVEIK

SEQ ID NO: 31 - protein, 115 aa, synthetic construct
  source 1..115: note = Codrituzumab VH
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 32 - protein, 112 aa, synthetic construct
  source 1..112: note = Codrituzumab VL
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 33 - protein, 117 aa, synthetic construct
  source 1..117: note = BMS-986182 VH
EVQLVQSGAEVKKPGESLKISCKGSGYSFTSYWIAWVRQMPGKGLEWMGIIFPGDSDTRYSPSFQGQVTISADRSIRTAYLQWSSLKASDTALYYCARTREGYFDYWGQGTLVTVSS

SEQ ID NO: 34 - protein, 107 aa, synthetic construct
  source 1..107: note = BMS-986182 VL
EIVLTQSPGTLSLSPGERATLSCRAVQSVSSSYLAWYQQKPGQAPRLLIYGASSRATGIPDRFSGSGSGTDFTLTISRLEPEDFAVYYCQQYGSSPTFGGGTKVEIK

SEQ ID NO: 35 - protein, 444 aa, synthetic construct
  source 1..444: note = M3-H1L1 HC
EVQLVQSGAEVKKPGSSVKVSCKASGYTFTDYEMHWVRQAPGQGLEWIGAIDPETGDTAYNQKFKGRATLTADKSTSTAYMELSSLRSEDTAVYYCTRYFSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 219 aa, synthetic construct
  source 1..219: note = M3-H1L1 LC
DVQMTQSPSSVSASVGDRVTITCRSSQSLRHSNGNTYLQWYQQKPGKAPKLLIYKVSNRFSGVPSRFSGSGSGTDFTLTISSLQPEDFATYFCYQSKHVPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 444 aa, synthetic construct
  source 1..444: note = M3-H18L6 HC
QVQLVQSGAEVKKPGSSVKVSCKASGYTFTDYEMHWVKQAPGHGLEWIGAIDPETGDTAYNQKFKGKATLTADTSTSTAYMELSSLRSEDTAVYYCTRYFSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 219 aa, synthetic construct
  source 1..219: note = M3-H18L6 LC
EIVLTQSPATLSLSPGERATISCRSSQSLRHSNGNTYLQWYQQKPGQAPRLLIYKVSNRFSGVPARFSGSGSGTDFTLTISSLEPEDFAVYFCYQSKHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 444 aa, synthetic construct
  source 1..444: note = Codrituzumab HC
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 219 aa, synthetic construct
  source 1..219: note = Codrituzumab LC
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 446 aa, synthetic construct
  source 1..446: note = BMS-986182 HC
EVQLVQSGAEVKKPGESLKISCKGSGYSFTSYWIAWVRQMPGKGLEWMGIIFPGDSDTRYSPSFQGQVTISADRSIRTAYLQWSSLKASDTALYYCARTREGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 214 aa, synthetic construct
  source 1..214: note = BMS-986182 LC
EIVLTQSPGTLSLSPGERATLSCRAVQSVSSSYLAWYQQKPGQAPRLLIYGASSRATGIPDRFSGSGSGTDFTLTISRLEPEDFAVYYCQQYGSSPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 23 aa, synthetic construct
  source 1..23: note = signal peptide
MRPTWAWWLFLVLLLALWAPARG

SEQ ID NO: 44 - protein, 444 aa, synthetic construct; identical to SEQ ID NO: 35
  source 1..444: note = CL_#27287 M3-H1L1 Full Heavy Chain

SEQ ID NO: 45 - DNA, 1332 bp, synthetic construct
  source 1..1332: mol_type = other DNA; note = CL_#27287 Full Heavy Chain
gaggtgcagctggtgcagagcggagccgaggtgaagaagcccggcagctccgtgaaggtgtcttgcaaggccagcggctacaccttcacagactatgagatgcactgggtgcggcaggcccccggacagggactggagtggatcggagccatcgaccctgagaccggcgatacagcctacaaccagaagtttaagggcagggccaccctgacagccgacaagagcacctccacagcctatatggagctgtctagcctgcggtccgaggataccgccgtgtactattgtacaagatacttctcttttgcctattggggccagggcaccctggtgacagtgtcctctgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaactgctgggaggaccaagcgtgttcctgtttccacccaagcctaaagacaccctgatgatttcccggactcctgaggtcacctgcgtggtcgtggacgtgtctcacgaggaccccgaagtcaagttcaactggtacgtggatggcgtcgaagtgcataatgccaagaccaaaccccgggaggaacagtacaactctacctatagagtcgtgagtgtcctgacagtgctgcaccaggactggctgaatgggaaggagtataagtgtaaagtgagcaacaaagccctgcccgccccaatcgaaaaaacaatctctaaagcaaaaggacagcctcgcgaaccacaggtgtacactctgcctccatctcgggacgagctgactaagaaccaggtcagtctgacctgtctggtgaaaggattctatcccagcgatatcgctgtggagtgggaatccaatggccagcctgagaacaattacaagaccacaccccctgtgctggactctgatggcagtttctttctgtatagtaagctgaccgtcgataaatcacgatggcagcaggggaacgtgttcagctgttcagtgatgcacgaagccctgcacaaccattacacccagaagagcctgagcctgtctcccggc

SEQ ID NO: 46 - DNA, 345 bp, synthetic construct
  source 1..345: mol_type = other DNA; note = CL_#27287 VH
gaggtgcagctggtgcagagcggagccgaggtgaagaagcccggcagctccgtgaaggtgtcttgcaaggccagcggctacaccttcacagactatgagatgcactgggtgcggcaggcccccggacagggactggagtggatcggagccatcgaccctgagaccggcgatacagcctacaaccagaagtttaagggcagggccaccctgacagccgacaagagcacctccacagcctatatggagctgtctagcctgcggtccgaggataccgccgtgtactattgtacaagatacttctcttttgcctattggggccagggcaccctggtgacagtgtcctct

SEQ ID NO: 47 - protein, 219 aa, synthetic construct; identical to SEQ ID NO: 36
  source 1..219: note = CL_#26960 M3-H1L1 Full Light Chain

SEQ ID NO: 48 - DNA, 657 bp, synthetic construct
  source 1..657: mol_type = other DNA; note = CL_#26960 Full Light Chain
gacgtgcagatgacacagtccccaagctccgtgagcgcctccgtgggcgatcgggtgaccatcacatgcagatctagccagtctctgaggcacagcaacggcaatacctacctgcagtggtatcagcagaagcccggcaaggcccctaagctgctgatctacaaggtgtccaaccgcttctctggcgtgcctagccggttttctggcagcggctccggcaccgacttcaccctgacaatctcctctctgcagcctgaggatttcgccacatacttttgttatcagtctaagcacgtgccatatacctttggccagggcacaaaggtggagatcaagagaacagtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 49 - DNA, 336 bp, synthetic construct
  source 1..336: mol_type = other DNA; note = CL_#26960 VL
gacgtgcagatgacacagtccccaagctccgtgagcgcctccgtgggcgatcgggtgaccatcacatgcagatctagccagtctctgaggcacagcaacggcaatacctacctgcagtggtatcagcagaagcccggcaaggcccctaagctgctgatctacaaggtgtccaaccgcttctctggcgtgcctagccggttttctggcagcggctccggcaccgacttcaccctgacaatctcctctctgcagcctgaggatttcgccacatacttttgttatcagtctaagcacgtgccatatacctttggccagggcacaaaggtggagatcaag

SEQ ID NO: 50 - protein, 444 aa, synthetic construct; identical to SEQ ID NO: 37
  source 1..444: note = CL_#28294 M3-H18L6 Full Heavy Chain

SEQ ID NO: 51 - DNA, 1332 bp, synthetic construct
  source 1..1332: mol_type = other DNA; note = CL_#28294 Full Heavy Chain
caggtgcagctggtgcagagcggagccgaggtgaagaagcccggcagctccgtgaaggtgtcttgcaaggccagcggctacaccttcacagactatgagatgcactgggtgaagcaggcccccggacacggactggagtggatcggagccatcgaccctgagacaggcgataccgcctacaaccagaagtttaagggcaaggccaccctgacagccgacaccagcacatccaccgcctatatggagctgtctagcctgcggtccgaggatacagccgtgtactattgtaccagatacttctcttttgcctattggggccagggcacactggtgaccgtgtcctctgctagcactaaggggccttccgtgtttccactggctccctctagtaaatccacctctggaggcacagctgcactgggatgtctggtgaaggattacttccctgaaccagtcacagtgagttggaactcaggggctctgacaagtggagtccatacttttcccgcagtgctgcagtcaagcggactgtactccctgtcctctgtggtcaccgtgcctagttcaagcctgggcacccagacatatatctgcaacgtgaatcacaagccatcaaatacaaaagtcgacaagaaagtggagcccaagagctgtgataaaactcatacctgcccaccttgtccggcgccagaactgctgggaggaccaagcgtgttcctgtttccacccaagcctaaagacaccctgatgatttcccggactcctgaggtcacctgcgtggtcgtggacgtgtctcacgaggaccccgaagtcaagttcaactggtacgtggatggcgtcgaagtgcataatgccaagaccaaaccccgggaggaacagtacaactctacctatagagtcgtgagtgtcctgacagtgctgcaccaggactggctgaatgggaaggagtataagtgtaaagtgagcaacaaagccctgcccgccccaatcgaaaaaacaatctctaaagcaaaaggacagcctcgcgaaccacaggtgtacactctgcctccatctcgggacgagctgactaagaaccaggtcagtctgacctgtctggtgaaaggattctatcccagcgatatcgctgtggagtgggaatccaatggccagcctgagaacaattacaagaccacaccccctgtgctggactctgatggcagtttctttctgtatagtaagctgaccgtcgataaatcacgatggcagcaggggaacgtgttcagctgttcagtgatgcacgaagccctgcacaaccattacacccagaagagcctgagcctgtctcccggc

SEQ ID NO: 52 - DNA, 345 bp, synthetic construct
  source 1..345: mol_type = other DNA; note = CL_#28294 VH
caggtgcagctggtgcagagcggagccgaggtgaagaagcccggcagctccgtgaaggtgtcttgcaaggccagcggctacaccttcacagactatgagatgcactgggtgaagcaggcccccggacacggactggagtggatcggagccatcgaccctgagacaggcgataccgcctacaaccagaagtttaagggcaaggccaccctgacagccgacaccagcacatccaccgcctatatggagctgtctagcctgcggtccgaggatacagccgtgtactattgtaccagatacttctcttttgcctattggggccagggcacactggtgaccgtgtcctct

SEQ ID NO: 53 - protein, 219 aa, synthetic construct; identical to SEQ ID NO: 38
  source 1..219: note = CL_#28295 M3-H18L6 Full Light Chain

SEQ ID NO: 54 - DNA, 657 bp, synthetic construct
  source 1..657: mol_type = other DNA; note = CL_#28295 Full Light Chain
gagatcgtgctgacccagtccccagccacactgtccctgtctccaggagagagggcaaccatctcttgcagaagctcccagagcctgaggcactccaacggcaatacatacctgcagtggtatcagcagaagccaggacaggcccctcgcctgctgatctacaaggtgtctaaccgcttcagcggcgtgcccgcccggtttagcggctccggctctggcaccgacttcaccctgacaatctctagcctggagcctgaggatttcgccgtgtacttttgttatcagtccaagcacgtgccatatacctttggcggcggcacaaaggtggagatcaagcggacagtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 55 - DNA, 336 bp, synthetic construct
  source 1..336: mol_type = other DNA; note = CL_#28295 VL
gagatcgtgctgacccagtccccagccacactgtccctgtctccaggagagagggcaaccatctcttgcagaagctcccagagcctgaggcactccaacggcaatacatacctgcagtggtatcagcagaagccaggacaggcccctcgcctgctgatctacaaggtgtctaaccgcttcagcggcgtgcccgcccggtttagcggctccggctctggcaccgacttcaccctgacaatctctagcctggagcctgaggatttcgccgtgtacttttgttatcagtccaagcacgtgccatatacctttggcggcggcacaaaggtggagatcaag

SEQ ID NO: 56 - protein, 445 aa, synthetic construct
  source 1..445: note = CL_#29372 Full Heavy Chain
QVQLVQSGAEVKKPGSSVKVSCKASGYTFTDYEMHWVKQAPGHGLEWIGAIDPETGDTAYNQKFKGKATLTADTSTSTAYMELSSLRSEDTAVYYCTRYFSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - DNA, 1335 bp, synthetic construct
  source 1..1335: mol_type = other DNA; note = CL_#29372 Full Heavy Chain
caggtccagcttgtacaatccggggcagaggtcaagaagccaggcagcagcgttaaagtgtcctgcaaggcttctggatacacatttaccgattatgagatgcactgggtcaagcaggctcccgggcatggtctcgagtggattggcgccatcgaccctgaaacaggagataccgcctataaccagaagtttaaagggaaggccaccctgaccgctgatacatcaacatccaccgcctacatggagctcagttctttgcggagcgaggacactgccgtgtactactgtacccggtactttagcttcgcctattggggccagggcacactggtgacagtttcttccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagaaggttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaa

SEQ ID NO: 58 - protein, 219 aa, synthetic construct
  source 1..219: note = CL_#30542 G34Q Light Chain
EIVLTQSPATLSLSPGERATISCRSSQSLRHSNQNTYLQWYQQKPGQAPRLLIYKVSNRFSGVPARFSGSGSGTDFTLTISSLEPEDFAVYFCYQSKHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - DNA, 657 bp, synthetic construct
  source 1..657: mol_type = other DNA; note = CL_#30542 G34Q Light Chain
gagatcgtgctgacccagtccccagccacactgagcctgtccccaggagagagggcaaccatctcctgcagaagctcccagtctctgaggcactctaaccagaacacatacctgcagtggtatcagcagaagccaggacaggcccctcgcctgctgatctacaaggtgtctaatcgcttcagcggcgtgcccgcccggttttctggcagcggctccggcaccgacttcaccctgacaatctcctctctggagcctgaggatttcgccgtgtacttttgttatcagagcaagcacgtgccatatacctttggcggcggcacaaaggtggagatcaagcggacagtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 60 - protein, 112 aa, synthetic construct
  source 1..112: note = CL_#30542 G34Q VL
EIVLTQSPATLSLSPGERATISCRSSQSLRHSNQNTYLQWYQQKPGQAPRLLIYKVSNRFSGVPARFSGSGSGTDFTLTISSLEPEDFAVYFCYQSKHVPYTFGGGTKVEIK

SEQ ID NO: 61 - DNA, 336 bp, synthetic construct
  source 1..336: mol_type = other DNA; note = CL_#30542 G34Q VL
gagatcgtgctgacccagtccccagccacactgagcctgtccccaggagagagggcaaccatctcctgcagaagctcccagtctctgaggcactctaaccagaacacatacctgcagtggtatcagcagaagccaggacaggcccctcgcctgctgatctacaaggtgtctaatcgcttcagcggcgtgcccgcccggttttctggcagcggctccggcaccgacttcaccctgacaatctcctctctggagcctgaggatttcgccgtgtacttttgttatcagagcaagcacgtgccatatacctttggcggcggcacaaaggtggagatcaag

SEQ ID NO: 62 - protein, 219 aa, synthetic construct
  source 1..219: note = CL_#30544 G34K Full Light Chain
EIVLTQSPATLSLSPGERATISCRSSQSLRHSNKNTYLQWYQQKPGQAPRLLIYKVSNRFSGVPARFSGSGSGTDFTLTISSLEPEDFAVYFCYQSKHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - DNA, 657 bp, synthetic construct
  source 1..657: mol_type = other DNA; note = CL_#30544 G34K Full Light Chain
gagatcgtgctgacccagtccccagccacactgagcctgtccccaggagagagggcaaccatctcctgcagaagctcccagtctctgaggcactctaacaagaacacatacctgcagtggtatcagcagaagccaggacaggcccctcgcctgctgatctacaaggtgtctaatcgcttcagcggcgtgcccgcccggttttctggcagcggctccggcaccgacttcaccctgacaatctcctctctggagcctgaggatttcgccgtgtacttttgttatcagagcaagcacgtgccatatacctttggcggcggcacaaaggtggagatcaagcggacagtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 64 - protein, 112 aa, synthetic construct
  source 1..112: note = CL_#30544 G34K VL
EIVLTQSPATLSLSPGERATISCRSSQSLRHSNKNTYLQWYQQKPGQAPRLLIYKVSNRFSGVPARFSGSGSGTDFTLTISSLEPEDFAVYFCYQSKHVPYTFGGGTKVEIK

SEQ ID NO: 65 - DNA, 336 bp, synthetic construct
  source 1..336: mol_type = other DNA; note = CL_#30544 G34K VL
gagatcgtgctgacccagtccccagccacactgagcctgtccccaggagagagggcaaccatctcctgcagaagctcccagtctctgaggcactctaacaagaacacatacctgcagtggtatcagcagaagccaggacaggcccctcgcctgctgatctacaaggtgtctaatcgcttcagcggcgtgcccgcccggttttctggcagcggctccggcaccgacttcaccctgacaatctcctctctggagcctgaggatttcgccgtgtacttttgttatcagagcaagcacgtgccatatacctttggcggcggcacaaaggtggagatcaag

SEQ ID NO: 66 - protein, 219 aa, synthetic construct
  source 1..219: note = CL_#30545 G34R Full Light Chain
EIVLTQSPATLSLSPGERATISCRSSQSLRHSNRNTYLQWYQQKPGQAPRLLIYKVSNRFSGVPARFSGSGSGTDFTLTISSLEPEDFAVYFCYQSKHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - DNA, 657 bp, synthetic construct
  source 1..657: mol_type = other DNA; note = CL_#30545 G34R Full Light Chain
gagatcgtgctgacccagtccccagccacactgagcctgtccccaggagagagggcaaccatctcctgcagaagctcccagtctctgaggcactctaacagaaacacatacctgcagtggtatcagcagaagccaggacaggcccctcgcctgctgatctacaaggtgtctaatcgcttcagcggcgtgcccgcccggttttctggcagcggctccggcaccgacttcaccctgacaatctcctctctggagcctgaggatttcgccgtgtacttttgttatcagagcaagcacgtgccatatacctttggcggcggcacaaaggtggagatcaagcggacagtggcggcgcccagtgtcttcatttttccccctagcgacgaacagctgaagtctgggacagccagtgtggtctgtctgctgaacaacttctaccctagagaggctaaagtgcagtggaaggtcgataacgcactgcagtccggaaattctcaggagagtgtgactgaacaggactcaaaagatagcacctattccctgtcaagcacactgactctgagcaaggccgactacgagaagcataaagtgtatgcttgtgaagtcacccaccaggggctgagttcaccagtcacaaaatcattcaacagaggggagtgc

SEQ ID NO: 68 - protein, 112 aa, synthetic construct
  source 1..112: note = CL_#30545 G34R VL
EIVLTQSPATLSLSPGERATISCRSSQSLRHSNRNTYLQWYQQKPGQAPRLLIYKVSNRFSGVPARFSGSGSGTDFTLTISSLEPEDFAVYFCYQSKHVPYTFGGGTKVEIK

SEQ ID NO: 69 - DNA, 336 bp, synthetic construct
  source 1..336: mol_type = other DNA; note = CL_#30545 G34R VL
gagatcgtgctgacccagtccccagccacactgagcctgtccccaggagagagggcaaccatctcctgcagaagctcccagtctctgaggcactctaacagaaacacatacctgcagtggtatcagcagaagccaggacaggcccctcgcctgctgatctacaaggtgtctaatcgcttcagcggcgtgcccgcccggttttctggcagcggctccggcaccgacttcaccctgacaatctcctctctggagcctgaggatttcgccgtgtacttttgttatcagagcaagcacgtgccatatacctttggcggcggcacaaaggtggagatcaag

SEQ ID NO: 70 - protein, 11 aa, synthetic construct
  source 1..11: note = IMGT LCDR1 (G34R)
QSLRHSNRNTY

SEQ ID NO: 71 - protein, 16 aa, synthetic construct
  source 1..16: note = Kabat, Chothia, AbM LCDR1 (G34R)
RSSQSLRHSNRNTYLQ

SEQ ID NO: 72 - protein, 12 aa, synthetic construct
  source 1..12: note = Contact LCDR1 (G34R)
RHSNRNTYLQWY

SEQ ID NO: 73 - protein, 11 aa, synthetic construct
QSLRHSNKNTY

SEQ ID NO: 74 - protein, 16 aa, synthetic construct
  source 1..16: note = Kabat, Chothia, AbM LCDR1 (G34K)
RSSQSLRHSNKNTYLQ

SEQ ID NO: 75 - protein, 12 aa, synthetic construct
  source 1..12: note = Contact LCDR1 (G34K)
RHSNKNTYLQWY

SEQ ID NO: 76 - protein, 11 aa, synthetic construct
  source 1..11: note = IMGT LCDR1 (G34Q)
QSLRHSNQNTY

SEQ ID NO: 77 - protein, 16 aa, synthetic construct
  source 1..16: note = Kabat, Chothia, AbM LCDR1 (G34Q)
RSSQSLRHSNQNTYLQ

SEQ ID NO: 78 - protein, 12 aa, synthetic construct
  source 1..12: note = Contact LCDR1 (G34Q)
RHSNQNTYLQWY

(19) United States
(12) Patent Application Publication
(10) Pub. No.: US 2025/0295799 A1
(43) Pub. Date: Sep. 25, 2025

(54) ANTIBODY-DRUG CONJUGATES TARGETING GLYPICAN-3 AND METHODS OF USE

(71) Applicant: Zymeworks BC Inc., Vancouver (CA)

(72) Inventors: Andrea Hernandez Rojas, Vancouver (CA); Chayne L. Piscitelli, Vancouver (CA); Stuart Daniel Barnscher, Vancouver (CA); James R. Rich, Vancouver (CA); Michael G. Brant, Vancouver (CA); Raffaele Colombo, Vancouver (CA); Samir Das, Vancouver (CA); Manuel Michel Auguste Lasalle, Vancouver (CA); Mark Edmund Petersen, Vancouver (CA); Alex Man Lai Wu, Vancouver (CA); Diego Arturo Alonzo Muniz, Vancouver (CA); Dunja Urosev, Vancouver (CA)

(21) Appl. No.: 19/173,694

(22) Filed: Apr. 8, 2025

Related U.S. Application Data
(63) Continuation of application No. PCT/CA2023/051378, filed on Oct. 18, 2023.
(60) Provisional application No. 63/417,295, filed on Oct. 18, 2022.
(60) Provisional application No. 63/458,857, filed on Apr. 12, 2023.

(51) Int. Cl. A61K 47/68; A61P 35/00; C07K 16/30

(52) U.S. Cl. CPC A61K 47/68037; A61K 47/6859; A61K 47/6889; A61P 35/00; C07K 16/303

(57) ABSTRACT

Described herein are antibody-drug conjugates (ADCs) comprising an antibody construct that binds human glypican 3 (GPC3) conjugated to a camptothecin analogue of Formula (I). The ADCs are useful as therapeutics, in particular in the treatment of cancer.

### CROSS REFERENCE TO RELATED APPLICATIONS

This application is a continuation of International Application No. PCT/CA2023/051378 filed on Oct. 18, 2023, which claims the benefit of U.S. Provisional Application No. 63/458,857, filed Apr. 12, 2023, and U.S. Provisional Application No. 63/417,295, filed Oct. 18, 2022, which is hereby incorporated in its entirety by reference.

### SEQUENCE LISTING

The instant application contains a Sequence Listing which is hereby incorporated by reference in its entirety. Said XML copy, created on Nov. 27, 2023, is named 2023 11 15 ZYME095WO.xml, and is 100,324 bytes in size.

### FIELD

The present disclosure relates to the field of immunotherapeutics and, in particular, to antibody-drug conjugates targeting human glypican-3 (GPC3).

### BACKGROUND

Glypican-3 (GPC3) is a glycosyl-phosphatidylinositol (GPI)-anchored oncofetal protein expressed on the surface of placental and fetal tissue such as liver, lung and kidney. GPC3 expression is downregulated or silenced in normal adult tissues, but expressed in hepatocellular carcinomas, melanomas, squamous cell lung carcinomas, and hepatoblastomas.

Numerous antibodies binding to human GPC3 have been described. Many of these antibodies are being developed as T-cell engager, NK-cell engager, chimeric antigen receptor (CAR) T cell or NK cell, or bispecific antibody therapeutics for the treatment of cancer. International Patent Publication No. WO2021/226321 (Phanes Therapeutics) describes several anti-GPC3 paratopes that specifically bind to human GPC3.

Some antibodies targeting GPC3 have been tested clinically in a monospecific format i.e. as a bivalent IgG. Codrituzumab, also known as GC33 or RG-7686, has been studied in clinical trials in adults with hepatocellular carcinoma (HCC), and in combination with other therapeutic agents and although exhibiting a good safety profile, showed limited efficacy. A clinical trial of BMS-986183, an antibody-drug conjugate (ADC) of the anti-GPC3 antibody BMS-986182 (also known as GPC3.1 (BMS) or 4A6 (Medarex)) conjugated to a tubulysin drug moiety was started in patients with advanced HCC, but no efficacy was observed, and the trial was terminated.

Fu et al. (Hepatology. 2019 August; 70(2): 563-576) have described An ADC of the anti-GPC3 antibody YP7 conjugated to DNA damaging agents Duocarmycin SA and pyrrolobenzodiazepine (PBD) has been described (see Fu et al. (2019) Hepatology, 70(2): 563-576). YP7 conjugated to PBD dimer showed potency in cancer cell models in vitro and caused tumor remission in mouse tumor models but this ADC has yet to be clinically evaluated.

Camptothecin analogues have been developed as payloads for ADCs. Two such ADCs have been approved for treatment of cancer. Trastuzumab deruxtecan (Enhertu™) in which the camptothecin analogue, deruxtecan (Dxd), is conjugated to the anti-HER2 antibody, trastuzumab, via a cleavable tetrapeptide-based linker, and sacituzumab govitecan (Trodelvy™) in which the camptothecin analogue, SN-38, is conjugated to the anti-Trop-2 antibody, sacituzumab, via a hydrolysable, pH-sensitive linker.

Other camptothecin analogues and derivatives, as well as ADCs comprising them have been described. See, for example, International (PCT) Publication Nos. WO 2019/195665; WO 2019/236954; WO 2020/200880 and WO 2020/219287.

This background information is provided for the purpose of making known information believed by the applicant to be of possible relevance to the present disclosure. No admission is necessarily intended, nor should be construed, that any of the preceding information constitutes prior art against the claimed invention.

### SUMMARY

Described herein are antibody-drug conjugates targeting glypican-3 (GPC3) and methods of use. One aspect of the present disclosure relates to antibody-drug conjugate having Formula (X):

T-[L-(D)ₘ]ₙ (X)
wherein:
m is an integer between 1 and 4;
n is an integer between 1 and 10;
T is an anti-GPC3 (glypican-3) antibody construct, comprising an antigen-binding domain that binds to human GPC3, the antigen-binding domain comprising:
   a) a heavy chain CDR1 (HCDR1) amino acid sequence comprising the sequence as set forth in SEQ ID NO: 6, a heavy chain CDR2 (HCDR2) amino acid sequence comprising the sequence as set forth in SEQ ID NO: 7, and a heavy chain CDR3 (HCDR3) amino acid sequence comprising the sequence as set forth in SEQ ID NO: 8, and
   b) a light chain CDR1 (LCDR1) amino acid sequence comprising the sequence as set forth in SEQ ID NO: 18, a light chain CDR2 (LCDR2) amino acid sequence comprising the sequence as set forth in SEQ ID NO: 19, and a light chain CDR3 (LCDR3) amino acid sequence comprising the sequence as set forth in SEQ ID NO: 17;
L is a linker, and
D is a compound of Formula I:

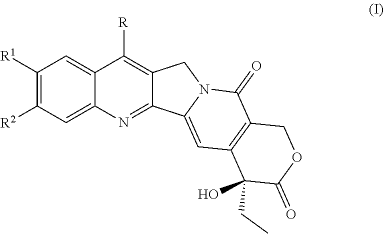

wherein:
   R¹ is selected from: —H, —CH₃, —CHF₂, —CF₃, —F, —Br, —Cl, —OH, —OCH₃, —OCF₃ and —NH₂, and
   R² is selected from: —H, —CH₃, —CF₃, —F, —Br, —Cl, —OH, —OCH₃ and —OCF₃, and wherein:
   when R¹ is-NH₂, then R is R³ or R⁴, and when R¹ is other than-NH₂, then R is R⁴;
   R³ is selected from: —H, —C₁-C₆ alkyl, —C₃-C₈ cycloalkyl, —(C₁-C₆ alkyl)-O—R⁵,

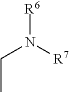

—CO₂R⁸, -aryl, -heteroaryl and —(C₁-C₆ alkyl)-aryl;
R⁴ is selected from:

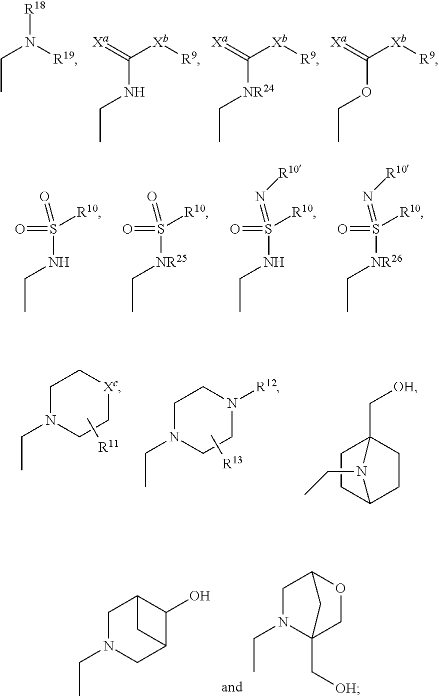

R⁵ is selected from: —H, —C₁-C₆ alkyl, —C₃-C₈ cycloalkyl, -aryl, -heteroaryl, -aryl and —(C₁-C₆ alkyl)-aryl;
R⁶ and R⁷ are each independently selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, —NR¹⁴R^{14′},-aryl, -heteroaryl and —(C₁-C₆ alkyl)-aryl;
R^{10′}is selected from: —H, —C₁-C₆ alkyl, —C₃-C₈ cycloalkyl, -aryl, -heteroaryl, and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl,—(C₁-C₆ alkyl)-aryl,-S(O)₂R¹⁶ and

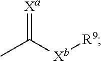

R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁷ is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R¹⁸ and R¹⁹ taken together with the N atom to which they are bonded form a 4-, 5-, 6- or 7-membered ring having 0 to 3 substituents selected from: halogen, —C₁-C₆ alkyl, —C₃-C₈ cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, O and S, and
X^{c} is selected from; O, S and S(O)₂,
with the proviso that the compound is other than (S)-9-amino-11-butyl-4-ethyl-4-hydroxy-1,12-dihydro-14H-pyrano[3′,4′:6,7]indolizino[1,2-b]quinoline-3,14(4H)-dione.

Another aspect of the present disclosure relates to an antibody-drug conjugate having the structure:

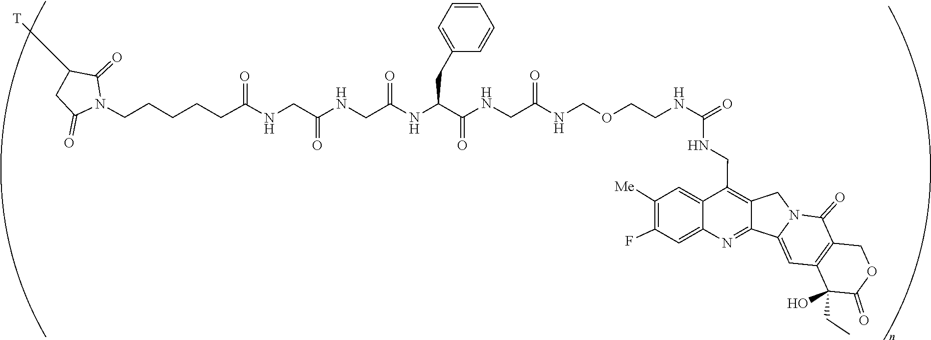

wherein:
n is between 1 and 10, and
T is an anti-GPC3 (glypican-3) antibody construct, comprising an antigen-binding domain that binds to human GPC3, the antigen-binding domain comprising:
   a) a heavy chain CDR1 (HCDR1) amino acid sequence comprising the sequence as set forth in SEQ ID NO: 6, a heavy chain CDR2 (HCDR2) amino acid sequence comprising the sequence as set forth in SEQ ID NO: 7, and a heavy chain CDR3 (HCDR3) amino acid sequence comprising the sequence as set forth in SEQ ID NO: 8, and
   b) 1) a light chain CDR1 (LCDR1) amino acid sequence comprising the sequence as set forth in SEQ ID NO: 71, a light chain CDR2 (LCDR2) amino acid sequence comprising the sequence as set forth in SEQ ID NO: 19, and a light chain CDR3 (LCDR3) amino acid sequence comprising the sequence as set forth in SEQ ID NO: 17; or
      ii) a light chain CDR1 (LCDR1) amino acid sequence comprising the sequence as set forth in SEQ ID NO: 74, a light chain CDR2 (LCDR2) amino acid sequence comprising the sequence as set forth in SEQ ID NO: 19, and a light chain CDR3 (LCDR3) amino acid sequence comprising the sequence as set forth in SEQ ID NO: 17; or
      iii) a light chain CDR1 (LCDR1) amino acid sequence comprising the sequence as set forth in SEQ ID NO: 77, a light chain CDR2 (LCDR2) amino acid sequence comprising the sequence as set forth in SEQ ID NO: 19, and a light chain CDR3 (LCDR3) amino acid sequence comprising the sequence as set forth in SEQ ID NO: 17.

Another aspect of the present disclosure relates to a pharmaceutical composition comprising an antibody-drug conjugate as described herein, and a pharmaceutically acceptable carrier or diluent.

Another aspect of the present disclosure relates to a method of inhibiting the proliferation of cancer cells comprising contacting the cells with an effective amount of the antibody-drug conjugate as described herein.

Another aspect of the present disclosure relates to a method of killing cancer cells comprising contacting the cells with an effective amount of the antibody-drug conjugate as described herein.

Another aspect of the present disclosure relates to a method of treating cancer in a subject in need thereof comprising administering to the subject an effective amount of the antibody-drug conjugate as described herein.

Another aspect of the present disclosure relates to an antibody-drug conjugate as described herein for use in the treatment of cancer.

Another aspect of the present disclosure relates to a use of an antibody-drug conjugate as described herein in the manufacture of a medicament for the treatment of cancer.

Another aspect of the present disclosure relates to a kit comprising an antibody-drug conjugate as described herein and a label and/or package insert containing instructions for use.

### BRIEF DESCRIPTION OF THE DRAWINGS

FIG. 1A shows the Caliper electrophoresis results under reducing (R) and non-reducing (NR) conditions for v37575 (codrituzumab), v37574 (M3-H18L6), and v33624 (BMS-986182).

FIG. 1B shows the UPLC-SEC profiles for the v37574 and v37575 (post SEC purification) and for v33624 (post Protein A purification).

FIG. 2 shows assessment of binding cross-reactivity of humanized antibody M3-H1L1 (v36180) to GPC1, GPC2, GPC3, and GPC5 as assessed by ELISA.

FIG. 3A shows binding of v36180 (M3-H1L1), v37574 (M3-H18L6), and codrituzumab compared to the control palivizumab in HepG2 cells. FIG. 3B depicts the binding of these same antibodies in JHH-7 cells.

FIG. 4A depicts the cytotoxicity of anti-GPC3 ADCs relative to non-targeting controls in GPC3-high HepG2 cells. FIG. 4B depicts the cytotoxicity of anti-GPC3 ADCs relative to non-targeting controls in GPC3-mid JHH-7 cells.

FIG. 5A shows the cytotoxicity of M3-H18L6 ADCs compared to non-targeting controls in GPC3-high HepG2 spheroids. FIG. 5B shows the cytotoxicity of M3-H18L6 ADCs compared to non-targeting controls in GPC3-mid NCI-H446 spheroids compared to non-targeting controls.

FIG. 6A shows the cytotoxicity of M3-H18L6 ADCs compared to a BMS-986182 ADC and a non-targeting antibody ADC in JHH-7 cells. FIG. 6B shows the cytotoxicity of M3-H18L6 ADCs compared to a BMS-986182 ADC and a non-targeting antibody ADC in JHH-7 spheroids cells.

FIG. 7 depicts the stability of M3-H1L1 and BMS-986182 ADCs in mouse plasma.

FIG. 8 shows the pharmacokinetic (PK) profile of M3-H18L6 and M3-H1L1 antibodies and ADCs of these antibodies in a Tg32 mouse model.

FIG. 9A shows a comparison of the efficacy of ADCs of BMS-986182 and M3-H1L1 in a JHH-7 cell line-derived xenograft model. FIG. 9B shows a comparison of the efficacy of the same ADCs in an NCI-H446 cell line-derived xenograft model.

FIG. 10A shows the PK profile of M3-H1L1 ADCs in an NCI-H446 xenograft model.

FIG. 10B shows the PK profile of M3-H1L1 ADCs in an NCI-H446 xenograft model in a JHH-7 cell line-derived xenograft model.

FIG. 11A shows the efficacy of M3-H1L1 and M3-H18L6 ADCs in a JHH-7 cell line-derived xenograft model. FIG. 11B shows the efficacy of M3-H1L1 and M3-H18L6 ADCs in an NCI-H446 cell line-derived xenograft model.

FIG. 12A depicts the efficacy of M3-H18L6 ADCs in a HepG2 xenograft model. FIG. 12B depicts the efficacy of M3-H18L6 ADCs in a Hep3B xenograft model. FIG. 12C depicts the efficacy of M3-H18L6 ADCs in a Huh-7 xenograft model. FIG. 12D depicts the efficacy of M3-H18L6 ADCs in a PLC/PRF/5 xenograft model.

FIG. 13A depicts the efficacy of M3-H18L6 ADCs in a LI1025 patient-derived xenograft model. FIG. 13B depicts the efficacy of M3-H18L6 ADCs in a LI1037 patient-derived xenograft model.

FIG. 14A shows the bystander effect of ADCs of v37574 (M3-H18L6) and v37575 (codrituzumab) in co-culture with GPC3-high HepG2 cells. FIG. 14B shows the bystander effect of ADCs of v37574 (M3-H18L6) and v37575 (codrituzumab) in co-culture with GPC3-mid JHH-5 cells.

FIG. 15 shows the Membrane Proteome Array™ screening results for humanized variant v38592 in HEK293T cells).

FIG. 16A depicts binding of M3-H18L6 antibody and ADCs to CHO cells transfected with human GPC3. FIG. 16B depicts binding of M3-H18L6 antibody and ADCs to CHO cells transfected with cynomolgus monkey GPC3.

FIG. 17A depicts binding of M3-H18L6 antibody and ADCs to HepG2 cells. FIG. 17B depicts binding of M3-H18L6 antibody and ADCs to JHH-7 cells. FIG. 17C depicts binding of M3-H18L6 antibody and ADCs to JHH-5 cells. FIG. 17D depicts binding of M3-H18L6 antibody and ADCs to SNU-601 cells.

FIG. 18A depicts in vivo efficacy of M3-H18L6 ADCs in a JHH-7 CDX model. FIG. 18B depicts in vivo efficacy of M3-H18L6 ADCs in a Hep3B CDX model. FIG. 18C depicts in vivo efficacy of M3-H18L6 ADCs in a JHH-5 CDX model.

FIG. 19A depicts in vivo efficacy of M3-H18L6 ADCs in a LI0050 PDX model of hepatocellular carcinoma (HCC). FIG. 19B depicts in vivo efficacy of M3-H18L6 ADCs in a LI1005 PDX model of HCC. FIG. 19C depicts in vivo efficacy of M3-H18L6 ADCs in a LI1069 PDX model of HCC. FIG. 19D depicts in vivo efficacy of M3-H18L6 ADCs in a LI1097 PDX model of HCC. FIG. 19E depicts in vivo efficacy of M3-H18L6 ADCs in a L16610 PDX model of HCC. FIG. 19F depicts in vivo efficacy of M3-H18L6 ADCs in a LI6619 PDX model of HCC.

FIG. 19G depicts in vivo efficacy of M3-H18L6 ADCs in a LI6677 PDX model of HCC.

FIG. 20 depicts the pharmacokinetic (PK) profile of v38592-MC-GGFG-AM-Compound 139 at DAR4.

FIG. 21 depicts the pharmacokinetic (PK) profile of v38592-MC-GGFG-AM-Compound 139 at DAR8.

### DETAILED DESCRIPTION

The present disclosure relates to antibody-drug conjugates (ADCs) comprising an antibody construct that binds to human glypican-3 GPC3 (an anti-GPC3 antibody construct) conjugated to a camptothecin analogue of Formula (I) as described herein. The ADCs of the present disclosure may find use, for example, as therapeutics, in particular in the treatment of cancer.

### Definitions

Unless defined otherwise, all technical and scientific terms used herein have the same meaning as commonly understood by one of ordinary skill in the art.

As used herein, the term “about” refers to an approximately +/−10% variation from a given value. It is to be understood that such a variation is always included in any given value provided herein, whether or not it is specifically referred to.

The use of the word “a” or “an” when used herein in conjunction with the term “comprising” may mean “one,” but it is also consistent with the meaning of “one or more,” “at least one” and “one or more than one.”

Where a range of values is provided herein, for example where a value is defined as being “between” an upper limit value and a lower limit value, it is understood that the range encompasses both the upper limit value and the lower limit value as well as each intervening value.

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

A “complementarity determining region” or “CDR” is an amino acid sequence that contributes to antigen-binding specificity and affinity. “Framework” regions (FR) can aid in maintaining the proper conformation of the CDRs to promote binding between the antigen-binding region and an antigen. From N-terminus to C-terminus, both the light chain variable region (VL) and the heavy chain variable region (VH) of an antibody typically comprise the domains FR1, CDR1, FR2, CDR2, FR3, CDR3, and FR4. The three heavy chain CDRs are referred to herein as HCDR1, HCDR2, and HCDR3, and the three light chain CDRs are referred to as LCDR1, LCDR2, and LCDR3. CDRs provide the majority of contact residues for the binding of the antibody to the antigen or epitope. Often, the three heavy chain CDRs and the three light chain CDRs are required to bind antigen. However, in some instances, even a single variable domain can confer binding specificity to the antigen. Furthermore, as is known in the art, in some cases, antigen-binding may also occur through a combination of a minimum of one or more CDRs selected from the VH and/or VL domains, for example HCDR3.

A number of different definitions of the CDR sequences are in common use, including those described by Kabat et al. (1983, Sequences of Proteins of Immunological Interest, NIH Publication No. 369-847, Bethesda, MD), by Chothia et al. (1987, J Mol Biol, 196:901-917), as well as the IMGT, AbM (University of Bath) and Contact (MacCallum, et al., 1996, J Mol Biol, 262(5):732-745) definitions. By way of example, CDR definitions according to Kabat, Chothia, IMGT, AbM and Contact are provided in Table 1 below. Accordingly, as would be readily apparent to one skilled in the art, the exact numbering and placement of CDRs may differ based on the numbering system employed. However, it is to be understood that the disclosure herein of a VH includes the disclosure of the associated (inherent) heavy chain CDRs (HCDRs) as defined by any of the known numbering systems. Similarly, disclosure herein of a VL includes the disclosure of the associated (inherent) light chain CDRs (LCDRs) as defined by any of the known numbering systems.

TABLE 1
Common CDR Definitions¹

| | Heavy Chain | | | Light Chain | | |
|---|---|---|---|---|---|---|
| Definition | CDR1² | CDR2 | CDR3 | CDR1 | CDR2 | CDR3 |
| Kabat | H31-H35B | H50-H65 | H95-H102 | L24-L34 | L50-L56 | L89-L97 |
| Chothia | H26-H32, | H52-H56 | H95-H102 | L24-L34 | L50-L56 | L89-L97 |
| | H33 or H34 | | | | | |
| IMGT | H26-H33, | H51-H57 | H93-H102 | L27-L32 | L50-L52 | L89-L97 |
| | H34, H35, | | | | | |
| | H35A or | | | | | |
| | H35B | | | | | |
| AbM | H26-H35B | H50-H58 | H95-H102 | L24-L34 | L50-L56 | L89-L97 |
| Contact | H30-H35B | H47-H58 | H93-H101 | L30-L36 | L46-L55 | L89-L96 |

¹Either the Kabat or Chothia numbering system may be used for HCDR2, HCDR3 and the light chain CDRs for all definitions except Contact, which uses Chothia numbering
²Using Kabat numbering. The position in the Kabat numbering scheme that demarcates the end of the Chothia and IMGT CDR-H1 loop varies depending on the length of the loop because Kabat places insertions outside of those CDR definitions at positions 35A and 35B. However, the IMGT and Chothia CDR-H1 loop can be unambiguously defined using Chothia numbering. CDR-H1 definitions using Chothia numbering: Kabat H31-H35, Chothia H26-H32, AbM H26-H35, IMGT H26-H33, Contact H30-H35.

The term “identical” in the context of two or more polynucleotide or polypeptide sequences, refers to two or more sequences or subsequences that are the same. Sequences are “substantially identical” if they have a percentage of amino acid residues or nucleotides that are the same (for example, about 80%, about 85%, about 90%, about 95%, or about 98% identity, over a specified region) when compared and aligned for maximum correspondence over a comparison window or over a designated region as measured using one of the commonly used sequence comparison algorithms as known to persons of ordinary skill in the art or by manual alignment and visual inspection. For sequence comparison, typically test sequences are compared to a designated reference sequence. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window” refers to a segment of a sequence comprising contiguous amino acid or nucleotide positions which may be, for example, from about 10 to 600 contiguous amino acid or nucleotide positions, or from about 10 to about 200, or from about 10 to about 150 contiguous amino acid or nucleotide positions over which a test sequence may be compared to a reference sequence of the same number of contiguous positions after the two sequences are optimally aligned. Methods of alignment of sequences for comparison are known to those of ordinary skill in the art. Optimal alignment of sequences for comparison can be conducted, for example, by the local homology algorithm of Smith & Waterman, 1970, Adv. Appl. Math., 2:482c; by the homology alignment algorithm of Needleman & Wunsch, 1970, J. Mol. Biol., 48:443; by the search for similarity method of Pearson & Lipman, 1988, Proc. Natl. Acad. Sci. USA, 85:2444, or by computerized implementations of these algorithms (for example, GAP, BESTFIT, FASTA or TFASTA in the Wisconsin Genetics Software Package, Genetics Computer Group, Madison, WI), or by manual alignment and visual inspection (see, for example, Ausubel et al., Current Protocols in Molecular Biology, (1995 supplement), Cold Spring Harbor Laboratory Press). Examples of available algorithms suitable for determining percent sequence identity are the BLAST and BLAST 2.0 algorithms, which are described in Altschul et al., 1997, Nuc. Acids Res., 25:3389-3402, and Altschul et al., 1990, J. Mol. Biol., 215:403-410, respectively. Software for performing BLAST analyses is publicly available through the website for the National Center for Biotechnology Information (NCBI).

The term “acyl,” as used herein, refers to the group —C(O)R, where R is hydrogen, alkyl, aryl, heteroaryl, cycloalkyl or heterocycloalkyl.

The term “acyloxy” refers to the group —OC(O)R, where R is alkyl.

The term “alkoxy,” as used herein, refers to the group —OR, where R is alkyl, aryl, heteroaryl, cycloalkyl or cycloheteroalkyl.

The term “alkyl,” as used herein, refers to a straight chain or branched saturated hydrocarbon group containing the specified number of carbon atoms. Examples of alkyl include, but are not limited to, methyl, ethyl, n-propyl, isopropyl, n-butyl, sec-butyl, isobutyl, t-butyl, pentyl, isopentyl, t-pentyl, neo-pentyl, 1-methylbutyl, 2-methylbutyl, n-hexyl, and the like.

The term “alkylaminoaryl,” as used herein, refers to an alkyl group as defined herein substituted with one aminoaryl group as defined herein.

The term “alkylheterocycloalkyl,” as used herein, refers to an alkyl group as defined herein substituted with one heterocycloalkyl group as defined herein.

The term “alkylthio,” as used herein, refers to the group —SR, where R is an alkyl group.

The term “amido,” as used herein, refers to the group —C(O)NRR′, where R and R′ are independently hydrogen, alkyl, aryl, heteroaryl, cycloalkyl or heterocycloalkyl.

The term “amino,” as used herein, refers to the group —NRR′, where R and R′ are independently hydrogen, alkyl, aryl, heteroaryl, cycloalkyl or heterocycloalkyl.

The term “aminoalkyl,” as used herein, refers to an alkyl group as defined herein substituted with one or more amino groups, for example, one, two or three amino groups.

The term “aminoaryl,” as used herein, refers to an aryl group as defined herein substituted with one amino group.

The term “aryl,” as used herein, refers to a 6- to 12-membered mono- or bicyclic hydrocarbon ring system in which at least one ring aromatic. Examples of aryl include, but are not limited to, phenyl, naphthalenyl, 1,2,3,4-tetrahydro-naphthalenyl, 5,6,7,8-tetrahydro-naphthalenyl, indanyl, and the like.

The term “carboxy,” as used herein, refers to the group —C(O)OR, where R is H, alkyl, aryl, heteroaryl, cycloalkyl or cycloheteroalkyl.

The term “cyano,” as used herein, refers to the group —CN.

The term “cycloalkyl,” as used herein, refers to a mono- or bicyclic saturated hydrocarbon containing the specified number of carbon atoms. Examples of cycloalkyl include, but are not limited to, cyclopropyl, cyclobutyl, cyclopentyl, cyclohexyl, cycloheptane, bicyclo [2.2.1]heptane, bicyclo [3.1.1]heptane, and the like.

The term “haloalkyl,” as used herein, refers to an alkyl group as defined herein substituted with one or more halogen atoms.

The terms “halogen” and “halo,” as used herein refer to fluorine (F), bromine (Br), chlorine (Cl) and iodine (I).

The term “heteroaryl,” as used herein, refers to a 6- to 12-membered mono- or bicyclic ring system in which at least one ring atom is a heteroatom and at least one ring is aromatic.

Examples of heteroatoms include, but are not limited to, O, S and N. Examples of heteroaryl include, but are not limited to: pyridyl, benzofuranyl, pyrazinyl, pyridazinyl, pyrimidinyl, triazinyl, quinolinyl, benzoxazolyl, benzothiazolyl, isoquinolinyl, quinazolinyl, quinoxalinyl, pyrrolyl, indolyl, and the like.

The term “heterocycloalkyl,” as used herein, refers to a mono- or bicyclic non-aromatic ring system containing the specified number of atoms and in which at least one ring atom is a heteroatom, for example, O, S or N. A heterocyclyl substituent can be attached via any of its available ring atoms, for example, a ring carbon, or a ring nitrogen. Examples of heterocycloalkyl include, but are not limited to, aziridinyl, azetidinyl, piperidinyl, morpholinyl, piperazinyl, pyrrolidinyl, and the like.

The terms “hydroxy” and “hydroxyl,” as used herein, refer to the group —OH.

The term “hydroxyalkyl,” as used herein, refers to an alkyl group as defined herein substituted with one or more hydroxy groups.

The term “nitro,” as used herein, refers to the group —NO₂.

The term “sulfonyl,” as used herein, refers to the group —S(O)₂R, where R is H, alkyl or aryl.

The term “sulfonamido,” as used herein, refers to the group —NH—S(O)₂R, where R is H, alkyl or aryl.

The terms “thio” and “thiol,” as used herein, refer to the group —SH.

Unless specifically stated as being “unsubstituted,” any alkyl, cycloalkyl, heterocycloalkyl, aryl or heteroaryl group referred to herein is understood to be “optionally substituted,” i.e. each such reference includes both unsubstituted and substituted versions of these groups. For example, reference to a “—C₁-C₆ alkyl” includes both unsubstituted —C₁-C₆ alkyl and —C₁-C₆ alkyl substituted with one or more substituents. Examples of substituents include, but are not limited to, halogen, acyl, acyloxy, alkoxy, carboxy, hydroxy, amino, amido, nitro, cyano, azido, alkylthio, thio, sulfonyl, sulfonamido, alkyl, cycloalkyl, heterocycloalkyl, aryl or heteroaryl. In certain embodiments, each alkyl, cycloalkyl, heterocycloalkyl, aryl or heteroaryl group referred to herein is optionally substituted with one or more substituents selected from: halogen, acyl, acyloxy, alkoxy, carboxy, hydroxy, amino, amido, nitro, cyano, azido, alkylthio, thio, sulfonyl and sulfonamido.

A chemical group described herein as “substituted,” may include one substituent or a plurality of substituents up to the full valence of substitution for that group. For example, a methyl group may include 1, 2, or 3 substituents, and a phenyl group may include 1, 2, 3, 4, or 5 substituents. When a group is substituted with more than one substituent, the substituents may be the same or they may be different.

The term “subject,” as used herein, refers to an animal, in some embodiments a mammal, which is the object of treatment, observation or experiment. The animal may be a human, a non-human primate, a companion animal (for example, dog, cat, or the like), farm animal (for example, cow, sheep, pig, horse, or the like) or a laboratory animal (for example, rat, mouse, guinea pig, non-human primate, or the like). In certain embodiments, the subject is a human.

It is contemplated that any embodiment discussed herein can be implemented with respect to any method, use or composition disclosed herein, and vice versa.

Particular features, structures and/or characteristics described in connection with an embodiment disclosed herein may be combined with features, structures and/or characteristics described in connection with another embodiment disclosed herein in any suitable manner to provide one or more further embodiments.

It is also to be understood that the positive recitation of a feature in one embodiment, serves as a basis for excluding the feature in an alternative embodiment. For example, where a list of options is presented for a given embodiment or claim, it is to be understood that one or more option may be deleted from the list and the shortened list may form an alternative embodiment, whether or not such an alternative embodiment is specifically referred to.

### Antibody-Drug Conjugates

The present disclosure relates to antibody-drug conjugates (ADCs) comprising an anti-GPC3 antibody construct conjugated to a camptothecin analogue having Formula (I). In certain embodiments, the ADC has Formula (X):

T-[L-(D)ₘ]ₙ (X)
wherein:
T is an anti-GPC3 antibody construct as described herein;
L is a linker;
D is a camptothecin analogue having Formula (I);
m is an integer between 1 and 4, and
n is an integer between 1 and 10.

Components of Formula (X) are described below.

### Anti-GPC3 Antibody Constructs, “T”

The ADCs of the present disclosure comprise an anti-GPC3 antibody construct, T. In this context, the term “antibody construct” refers to a polypeptide or a set of polypeptides that comprises one or more antigen-binding domains, where each of the one or more antigen-binding domains specifically binds to an epitope or antigen. Where the antibody construct comprises two or more antigen-binding domains, each of the antigen-binding domains may bind the same epitope or antigen (i.e. the antibody construct is monospecific) or they may bind to different epitopes or antigens (i.e. the antibody construct is bispecific or multispecific). The antibody construct may further comprise a scaffold and the one or more antigen-binding domains can be fused or covalently attached to the scaffold, optionally via a linker.

In accordance with the present disclosure, the anti-GPC3 antibody construct comprises at least one antigen-binding domain that specifically binds to human GPC3 (hGPC3). By “specifically binds” to hGPC3, it is meant that the antibody construct binds to hGPC3 but does not exhibit significant binding to any of human glypican-1 (GPC1), glypican-2 (GPC2), glypican-4 (GPC4), glypican-5 (GPC5), or glypican-6 (GPC6). In one embodiment, the anti-GPC3 antibody construct binds to GPC3 but does not exhibit significant binding to any of GPC 1, GPC2, or GPC5. In certain embodiments, the anti-GPC3 antibody constructs of the present disclosure may be capable of binding to a GPC3 from one or more non-human species. In certain embodiments, the anti-GPC3 antibody constructs of the present disclosure are capable of binding to cynomolgus monkey GPC3.

Human GPC3 is also known as “Glypican Proteoglycan 3” or “Heparan Sulphate Proteoglycan.” The protein sequences of hGPC3 from various sources are known in the art and readily available from publicly accessible databases, such as Genflank or UniProtKB. Examples of hGPC3 sequences include for example those provided under NCBI reference numbers P51654, NP_001158091.1, NP 001158090.1, NP 001158089.1, NP_004475.1 and AAA98132.1. An exemplary hGPC3 protein sequence is provided in Table 2 as SEQ TD NO: 1 (NCBI Reference Sequence: P51654). An exemplary cynomolgus monkey GPC3 protein sequence is also provided in Table 2 (SEQ ID NO: 2; UniProt ID: AOA2K5VK50).

TABLE 2
Human and Cynomolgus Monkey GPC3 Protein Sequences

| | | SEQ |
|---|---|---|
| Organism | Sequence | ID NO |
| Human | QPPPPPPDATCHQVRSFFQRLQPGLKWVPETPVPGSDLQVCLPKGPT | 1 |
| | CCSRKMEEKYQLTARLNMEQLLQSASMELKFLIIQNAAVFQEAFEI | |
| | VVRHAKNYTNAMFKNNYPSLTPQAFEFVGEFFTDVSLYILGSDINV | |
| | DDMVNELFDSLFPVIYTQLMNPGLPDSALDINECLRGARRDLKVFG | |
| | NFPKLIMTQVSKSLQVTRIFLQALNLGIEVINTTDHLKFSKDCGRML | |
| | TRMWYCSYCQGLMMVKPCGGYCNVVMQGCMAGVVEIDKYWRE | |
| | YILSLEELVNGMYRIYDMENVLLGLFSTIHDSIQYVQKNAGKLTTTI | |
| | GKLCAHSQQRQYRSAYYPEDLFIDKKVLKVAHVEHEETLSSRRREL | |
| | IQKLKSFISFYSALPGYICSHSPVAENDTLCWNGQELVERYSQKAAR | |
| | NGMKNQFNLHELKMKGPEPVVSQIIDKLKHINQLLRTMSMPKGRV | |
| | LDKNLDEEGFESGDCGDDEDECIGGSGDGMIKVKNQLRFLAELAY | |
| | DLDVDDAPGNSQQATPKDNEISTFHNLGNVHSPLKLLTSMAISVVC | |
| | FFFLVH | |
| Cynomolgus | QPPPPPPDATCHQVRSFFQRLQPGLKWVPETPVPGSDLQVCLPKGPT | 2 |
| Monkey | CCSRKMEEKYQLTARLNMEQLLQSASMELKFLIIQNAAVFQEAFEI | |
| | VVRHAKNYTNAMFKNNYPSLTPQAFEFVGEFFTDVSLYILGSDINV | |
| | DDMVNELFDSLFPVIYTQLMNPGLPDSALDINECLRGARRDLKVFG | |
| | NFPKLIMTQVSKSLQVTRIFLQALNLGIEVINTTDHLKFSKDCGRML | |
| | TRMWYCSYCQGLMMVKPCGGYCNVVMQGCMAGVVEIDKYWRE | |
| | YILSLEELVNGMYRIYDMENVLLGLFSTIHDSIQYVQKNAGKLTTTI | |
| | GKLCAHSQQRQYRSAYYPEDLFIDKKVLKVAHVEHEETLSSRRREL | |
| | IQKLKSFISFYSALPGYICSHSPVAENDTLCWNGQELVERYSQKAAR | |
| | NGMKNQFNLHELKMKGPEPVVSQIIDKLKHINQLLRTMSVPKGRVL | |
| | DKNLDEEGFESGDCGDDEDECIGGSGDGMMKVKNQLRFLAELAYD | |
| | LDVDDVPGNNQQATPKDNEISTFHNLGNVHSPLKLLTSMAISVVCF | |
| | FFLVH | |

Specific binding of an antigen-binding domain to a target antigen or epitope may be measured, for example, through an enzyme-linked immunosorbent assay (ELISA), a surface plasmon resonance (SPR) technique (employing, for example, a BIAcore instrument) (Liljeblad et al., 2000, Glyco J, 17:323-329), flow cytometry or a traditional binding assay (Heeley, 2002, Endocr Res, 28:217-229). In certain embodiments, specific binding may be defined as the extent of binding to a non-target protein (such as GPC1, GPC2, or GPC5) being less than about 10% of the binding to hGPC3 as measured by ELISA or flow cytometry, for example.

The term “dissociation constant (K_{D} or K_{d})” as used herein, is intended to refer to the equilibrium dissociation constant of a particular ligand-protein interaction. As used herein, ligand-protein interactions refer to, but are not limited to protein-protein interactions or antibody-antigen interactions. The K_{D} measures the propensity of two proteins complexed together (e.g. AB) to dissociate reversibly into constituent components (A+B), and is defined as the ratio of the rate of dissociation, also called the “off-rate (k_{off})”, to the association rate, or “on-rate (kₒₙ.)”. Thus, K_{D} equals k_{off}/kₒₙ and is expressed as a molar concentration (M). It follows that the smaller the K_{D}, the stronger the affinity of binding, and thus a decrease in K_{D} indicates an increase in affinity. Therefore, a K_{D} of 1 mM indicates weak binding affinity compared to a K_{D} of 1 nM. Affinity is sometimes measured in terms of a K_{A} or Kₐ, which is the reciprocal of the K_{D} or K_{d}. K_{D} values for antibody constructs can be determined using methods well established in the art. One method for determining the K_{D} of an antibody construct is by using surface plasmon resonance (SPR), typically using a biosensor system such as a Biacore® system. Isothermal titration calorimetry (ITC) is another method that can be used to measure K_{D}. The Octet™ system may also be used to measure the affinity of antibodies for a target antigen.

In certain embodiments, specific binding of an antibody construct for GPC3 may be defined by a dissociation constant (K_{D}) of ≤1 μM, for example, ≤500 nM, ≤250 nM, ≤100 nM, ≤50 nM, or ≤10 nM. In certain embodiments, specific binding of an antibody construct for a particular antigen or an epitope may be defined by a dissociation constant (K_{D}) of 10⁻⁶ M or less, for example, 10⁻⁷ M or less, or 10⁻⁸ M or less. In some embodiments, specific binding of an antibody construct for a particular antigen or an epitope may be defined by a dissociation constant (K_{D}) between 10⁻⁶ M and 10⁻⁹ M, for example, between 10⁻⁷ M and 10⁻⁹ M.

In some embodiments, the antigen-binding domain of the anti-GPC3 antibody construct binds to human GPC3 with a K_{D} that is higher than that of reference antibody codrituzumab, as measured by SPR. Accordingly, in these embodiments, the anti-GPC3 antibody constructs of the ADCs of the present disclosure comprise an antigen-binding domain having an affinity for human GPC3 that is lower than that of reference antibody codrituzumab.

The anti-GPC3 antibody constructs are internalized by GPC3-expressing cells. Antibody internalization may be measured using art-known methods, for example, by a direct internalization method according to the protocol detailed in Schmidt, M. et al., 2008, Cancer Immunol. Immunother., 57:1879-1890, or using commercially available fluorescent dyes such as the pHAb Dyes (Promega Corporation, Madison, WI), pHrodo iFL and Deep Red Dyes (ThermoFisher Scientific Corporation, Waltham, MA) and Incucyte® Fabfluor-pH Antibody Labeling Reagent (Sartorius A G, Gottingen, Germany) and analysis techniques such as microscopy, FACS, high content imaging or other plate-based assays.

In some embodiments, the anti-GPC3 antibody construct is internalized to a similar extent as reference antibody codrituzumab in cells expressing GPC3 at a high level, for example in HepG2 cells, or in JHH-7 cells. In some embodiments, the amount of internalized antibody is determined after at least a 5-hour incubation period. In some embodiments, conjugation of the anti-GPC3 antibody construct to a camptothecin analogue does not affect internalization of the anti-GPC3 antibody construct.

GPC3 expression varies depending on cell type as indicated throughout the disclosure and the level of GPC3 expression is sometimes referred to herein as “high”, “mid,” “low” or “negative.” These terms are used for reference to describe levels of GPC3 expression in general according to the designations shown in Table 12.1 in Example 12 and are not intended to be limited to the specific numerical values for average GPC3 per cell included therein. Alternatively, expression level of GPC3 in cells or tumors may be assessed by immunohistochemistry (IHC) according to methods known in the art. For example, IHC may be used to stain for GPC3 in tumor tissue samples from xenograft models, cell line-derived (CDX) or patient-derived (PDX). Tissue samples may be examined, and an H-score calculated as known in the art and described, for example in Example 33, herein. The higher the H-score, the higher the expression of GPC3 in the tissue sample.

### Antigen-Binding Domains

The anti-GPC3 antibody constructs of the present disclosure comprise at least one antigen-binding domain that is capable of binding to hGPC3. At least one antigen-binding domain capable of binding to hGPC3 typically is an immunoglobulin-based binding domain, such as an antigen-binding antibody fragment. Examples of an antigen-binding antibody fragment include, but are not limited to, a Fab fragment, a Fab′ fragment, a single chain Fab (scFab), a single chain Fv (scFv) and a single domain antibody (sdAb).

A “Fab fragment” contains the constant domain of the light chain (CL) and the first constant domain of the heavy chain (CH1) along with the variable domains of the light and heavy chains (VL and VH, respectively). Fab′ fragments differ from Fab fragments by the addition of a few amino acid residues at the C-terminus of the heavy chain CH1 domain, including one or more cysteines from the antibody hinge region. A Fab fragment may also be a single-chain Fab molecule, i.e. a Fab molecule in which the Fab light chain and the Fab heavy chain are connected by a peptide linker to form a single peptide chain. For example, the C-terminus of the Fab light chain may be connected to the N-terminus of the Fab heavy chain in the single-chain Fab molecule.

An “scFv” includes a heavy chain variable domain (VH) and alight chain variable domain (VL) of an antibody in a single polypeptide chain. The scFv may optionally further comprise a polypeptide linker between the VH and VL domains which enables the scFv to form a desired structure for antigen binding. For example, an scFv may include a VL connected from its C-terminus to the N-terminus of a VH by a polypeptide linker. Alternately, an scFv may comprise a VH connected through its C-terminus to the N-terminus of a VL by a polypeptide linker (see review in Pluckthun in The Pharmacology ofMonoclonal Antibodies, vol. 113, Rosenburg and Moore eds., Springer-Verlag, New York, pp. 269-315 (1994)).

An “sdAb” format refers to a single immunoglobulin domain. The sdAb may be, for example, of camelid origin. Camelid antibodies lack light chains and their antigen-binding sites consist of a single domain, termed a “VHH.” An sdAb comprises three CDR/hypervariable loops that form the antigen-binding site: CDR1, CDR2 and CDR3. sdAbs are fairly stable and easy to express, for example, as a fusion with the Fc chain of an antibody (see, for example, Harmsen & De Haard, 2007, Appl. Microbiol Biotechnol., 77(1):13-22).

In those embodiments in which the anti-GPC3 antibody constructs of the ADCs comprise two or more antigen-binding domains, each additional antigen-binding domain may independently be an immunoglobulin-based domain, such as an antigen-binding antibody fragment, or a non-immunoglobulin-based domain, such as a non-immunoglobulin-based antibody mimetic, or other polypeptide or small molecule capable of specifically binding to its target, for example, a natural or engineered ligand. Non-immunoglobulin-based antibody mimetic formats include, for example, anticalins, fynomers, affimers, alphabodies, DARPins and avimers.

In certain embodiments, the anti-GPC3 antibody constructs of the ADCs of the present disclosure comprise at least one antigen-binding domain that specifically binds to hGPC3, where the antigen-binding domain is derived from the MAb clone M3 described in WO2021/226321. Thus, in certain embodiments, the anti-GPC3 antibody construct of the ADCs of the present disclosure comprises a set of HCDRs and a set of LCDRs, identified according to IMGT, Kabat, Chothia AbM or Contact numbering, as set forth in Table 3 below.

TABLE 3
CDR amino acid sequences for MAb clone M3

| | Sequence | | | | | |
|---|---|---|---|---|---|---|
| | | SEQ | | SEQ | | SEQ |
| Numbering | Heavy Chain | ID | Heavy Chain | ID | Heavy Chain | ID |
| System | CDR1 | NO | CDR2 | NO | CDR3 | NO |
| IMGT | GYTFTDYE | 3 | IDPETGDT | 4 | TRYFSFAY | 5 |
| Kabat | DYEMH | 6 | AIDPETGDTAYNQKFKG | 7 | YFSFAY | 8 |
| Chothia | GYTFTDY | 9 | DPETGD | 10 | YFSFAY | 8 |
| AbM | GYTFTDYEMH | 11 | AIDPETGDTA | 12 | YFSFAY | 8 |
| Contact | TDYEMH | 13 | WIGAIDPETGDTA | 14 | TRYFSFA | 15 |
| | | SEQ | | SEQ | | SEQ |
| | Light Chain | ID | Light Chain | ID | Light Chain | ID |
| | CDR1 | NO | CDR2 | NO | CDR3 | NO |
| IMGT | QSLRHSNGNTY | 16 | KVS | — | YQSKHVPYT | 17 |
| Kabat | RSSQSLRHSNGNTYLQ | 18 | KVSNRFS | 19 | YQSKHVPYT | 17 |
| Chothia | RSSQSLRHSNGNTYLQ | 18 | KVSNRFS | 19 | YQSKHVPYT | 17 |
| AbM | RSSQSLRHSNGNTYLQ | 18 | KVSNRFS | 19 | YQSKHVPYT | 17 |
| Contact | RHSNGNTYLQWY | 20 | LLIYKVSNRF | 21 | YQSKHVPY | 22 |

In certain embodiments, the anti-GPC3 antibody construct of the ADCs of the present disclosure comprises an antigen-binding domain comprising the 3 HCDR amino acid sequences and the 3 LCDRs amino acid sequences of v36180 (M3-H1L1) or v37574 (M3-H18L6), as defined by IMGT, Kabat, Chothia or AbM numbering systems.

In certain embodiments, the anti-GPC3 antibody constructs of the ADCs of the present disclosure comprise an antigen-binding domain having a VH amino acid sequence that comprises the 3 HCDR amino acid sequences of v36180 (M3-H1L1) and a VL amino acid sequence that comprises the 3 LCDR amino acid sequences of v36180 (M3-H1L1). In certain other embodiments, the anti-GPC3 antibody constructs of the ADCs of the present disclosure comprise an antigen-binding domain having a VH amino acid sequence that comprises the 3 HCDR amino acid sequences of v37574 (M3-H18L6) and a VL amino acid sequence that comprises the 3 LCDR amino acid sequences of v37574 (M3-H18L6).

In certain embodiments, the anti-GPC3 antibody constructs of the ADCs of the present disclosure comprise an antigen-binding domain having heavy chain CDR amino acid sequences (HCDR1, HCDR2 and HCDR3) comprising the sequences as set forth in SEQ ID NOs: 6, 7 and 8, and light chain CDR amino acid sequences (LCDR1, LCDR2 and LCDR3) comprising the sequences as set forth in SEQ ID NOs: 18, 19 and 17 as defined by Kabat numbering.

In certain embodiments, the anti-GPC3 antibody constructs of the ADCs of the present disclosure comprise an antigen-binding domain having heavy chain CDR amino acid sequences (HCDR1, HCDR2 and HCDR3) comprising the sequences as set forth in SEQ ID NOs: 3, 4 and 5, and light chain CDR amino acid sequences (LCDR1 and LCDR3) comprising the sequences as set forth in SEQ ID NOs: 16 and 17 and the LCDR2 amino acid sequence KVS, as defined by IMGT numbering.

In certain embodiments, the anti-GPC3 antibody constructs of the ADCs of the present disclosure comprise an antigen-binding domain having heavy chain CDR amino acid sequences (HCDR1, HCDR2 and HCDR3) comprising the sequences as set forth in SEQ ID NOs: 9, 10 and 8, and light chain CDR amino acid sequences (LCDR1, LCDR2 and LCDR3) comprising the sequences as set forth in SEQ ID NOs: 18, 19 and 17 as defined by Chothia numbering.

In certain embodiments, the anti-GPC3 antibody constructs of the ADCs of the present disclosure comprise an antigen-binding domain having heavy chain CDR amino acid sequences (HCDR1, HCDR2 and HCDR3) comprising the sequences as set forth in SEQ ID NOs: 13, 14 and 15, and light chain CDR amino acid sequences (LCDR1, LCDR2 and LCDR3) comprising the sequences as set forth in SEQ ID NOs: 20, 21 and 22 as defined by Contact numbering.

In certain embodiments, the anti-GPC3 antibody constructs of the ADCs of the present disclosure comprise an antigen-binding domain having heavy chain CDR amino acid sequences (HCDR1, HCDR2 and HCDR3) comprising the sequences as set forth in SEQ ID NOs: 11, 12 and 8, and light chain CDR amino acid sequences (LCDR1, LCDR2 and LCDR3) comprising the sequences as set forth in SEQ ID NOs: 18, 19 and 17 as defined by AbM numbering.

One skilled in the art will appreciate that a limited number of amino acid substitutions may be introduced into the CDR sequences or into the VH or VL sequences of known antibodies without the antibody losing its ability to bind its target. Candidate amino acid substitutions may be identified by computer modeling or by art-known techniques such as alanine scanning, with the resulting variants being tested for binding activity by standard techniques. Accordingly, in certain embodiments, the anti-GPC3 antibody constructs of the ADCs of the present disclosure comprise an antigen-binding domain that comprises a set of CDRs (i.e. heavy chain HCDR1, HCDR2 and HCDR3, and light chain LCDR1, LCDR2 and LCDR3) that have 90% or greater, 95% or greater, 98% or greater, 99% or greater, or 100% sequence identity to the set of CDRs of v36180 (M3-H1L1) or v37574 (M3-H18L6), where the % sequence identity is calculated across all six CDRs and where the antigen-binding domain retains the ability to bind hGPC3.

In one embodiment, the anti-GPC3 antibody construct of the ADCs of the present disclosure comprises a set of HCDRs and a set of LCDRs as set forth in any one of Table 3A, Table 3B, or Table 3C below:

TABLE 3A
CDR amino acid sequences for Light chain-modified variants of MAb clone M3
(v40206, G34R)

| | Sequence | | | | | |
|---|---|---|---|---|---|---|
| | | SEQ | | SEQ | | SEQ |
| Numbering | Heavy Chain | ID | Heavy Chain | ID | Heavy Chain | ID |
| System | CDR1 | NO | CDR2 | NO | CDR3 | NO |
| IMGT | GYTFTDYE | 3 | IDPETGDT | 4 | TRYFSFAY | 5 |
| Kabat | DYEMH | 6 | AIDPETGDTAYNQKFKG | 7 | YFSFAY | 8 |
| Chothia | GYTFTDY | 9 | DPETGD | 10 | YFSFAY | 8 |
| AbM | GYTFTDYEMH | 11 | AIDPETGDTA | 12 | YFSFAY | 8 |
| Contact | TDYEMH | 13 | WIGAIDPETGDTA | 14 | TRYFSFA | 15 |
| | | SEQ | | SEQ | | SEQ |
| | Light Chain | ID | Light Chain | ID | Light Chain | ID |
| | CDR1 | NO | CDR2 | NO | CDR3 | NO |
| IMGT | QSLRHSNRNTY | 70 | KVS | - | YQSKHVPYT | 17 |
| Kabat | RSSQSLRHSNRNTYLQ | 71 | KVSNRFS | 19 | YQSKHVPYT | 17 |
| Chothia | RSSQSLRHSNRNTYLQ | 71 | KVSNRFS | 19 | YQSKHVPYT | 17 |
| AbM | RSSQSLRHSNRNTYLQ | 71 | KVSNRFS | 19 | YQSKHVPYT | 17 |
| Contact | RHSNRNTYLQWY | 72 | LLIYKVSNRF | 21 | YQSKHVPY | 22 |

TABLE 3B
CDR amino acid sequences for Light chain-modified variants of MAb clone M3
(v40207, G34K)

| | Sequence | | | | | |
|---|---|---|---|---|---|---|
| | | SEQ | | SEQ | | SEQ |
| Numbering | Heavy Chain | ID | Heavy Chain | ID | Heavy Chain | ID |
| System | CDR1 | NO | CDR2 | NO | CDR3 | NO |
| IMGT | GYTFTDYE | 3 | IDPETGDT | 4 | TRYFSFAY | 5 |
| Kabat | DYEMH | 6 | AIDPETGDTAYNQKFKG | 7 | YFSFAY | 8 |
| Chothia | GYTFTDY | 9 | DPETGD | 10 | YFSFAY | 8 |
| AbM | GYTFTDYEMH | 11 | AIDPETGDTA | 12 | YFSFAY | 8 |
| Contact | TDYEMH | 13 | WIGAIDPETGDTA | 14 | TRYFSFA | 15 |
| | | SEQ | | SEQ | | SEQ |
| | Light Chain | ID | Light Chain | ID | Light Chain | ID |
| | CDR1 | NO | CDR2 | NO | CDR3 | NO |
| IMGT | QSLRHSNKNTY | 73 | KVS | — | YQSKHVPYT | 17 |
| Kabat | RSSQSLRHSNKNTYLQ | 74 | KVSNRFS | 19 | YQSKHVPYT | 17 |
| Chothia | RSSQSLRHSNKNTYLQ | 74 | KVSNRFS | 19 | YQSKHVPYT | 17 |
| AbM | RSSQSLRHSNKNTYLQ | 74 | KVSNRFS | 19 | YQSKHVPYT | 17 |
| Contact | RHSNKNTYLQWY | 75 | LLIYKVSNRF | 21 | YQSKHVPY | 22 |

TABLE 3C
CDR amino acid sequences for Light chain (LC)-modified variants of MAb clone
M3 (v40208, G34Q)

| Numbering | Sequence | | | | | |
|---|---|---|---|---|---|---|
| System | | SEQ | | SEQ | | SEQ |
| | Heavy Chain | ID | Heavy Chain | ID | Heavy Chain | ID |
| | CDR1 | NO | CDR2 | NO | CDR3 | NO |
| IMGT | GYTFTDYE | 3 | IDPETGDT | 4 | TRYFSFAY | 5 |
| Kabat | DYEMH | 6 | AIDPETGDTAYNQKFKG | 7 | YFSFAY | 8 |
| Chothia | GYTFTDY | 9 | DPETGD | 10 | YFSFAY | 8 |
| AbM | GYTFTDYEMH | 11 | AIDPETGDTA | 12 | YFSFAY | 8 |
| Contact | TD YEMH | 13 | WIGAIDPETGDTA | 14 | TRYFSFA | 15 |
| | | SEQ | | SEQ | | SEQ |
| | Light Chain | ID | Light Chain | ID | Light Chain | ID |
| | CDR1 | NO | CDR2 | NO | CDR3 | NO |
| IMGT | QSLRHSNQNTY | 76 | KVS | — | YQSKHVPYT | 17 |
| Kabat | RSSQSLRHSNONTYLQ | 77 | KVSNRFS | 19 | YQSKHVPYT | 17 |
| Chothia | RSSQSLRHSNQNTYLQ | 77 | KVSNRFS | 19 | YQSKHVPYT | 17 |
| AbM | RSSQSLRHSNQNTYLQ | 77 | KVSNRFS | 19 | YQSKHVPYT | 17 |
| Contact | RHSNQNTYLQWY | 78 | LLIYKVSNRF | 21 | YQSKHVPY | 22 |

In certain embodiments, the anti-GPC3 antibody constructs of the ADCs of the present disclosure comprise an antigen-binding domain having a VH amino acid sequence that comprises the 3 HCDR amino acid sequences of LC-modified variant 40206 and a VL amino acid sequence that comprises the 3 LCDR amino acid sequences of v40206, as defined by one of IMGT, Kabat, Chothia, AbM, or Contact numbering. In certain other embodiments, the anti-GPC3 antibody constructs of the ADCs of the present disclosure comprise an antigen-binding domain having a VH amino acid sequence that comprises the 3 HCDR amino acid sequences of LC-modified variant 40207 and a VL amino acid sequence that comprises the 3 LCDR amino acid sequences of LC-modified variant 40207, as defined by one of IMGT, Kabat, Chothia, AbM, or Contact numbering. In still other embodiments, the anti-GPC3 antibody constructs of the ADCs of the present disclosure comprise an antigen-binding domain having a VH amino acid sequence that comprises the 3 HCDR amino acid sequences of LC-modified variant 40208 and a VL amino acid sequence that comprises the 3 LCDR amino acid sequences of LC-modified variant 40208, as defined by one of IMGT, Kabat, Chothia, AbM, or Contact numbering.

In certain embodiments, the anti-GPC3 antibody constructs of the ADCs of the present disclosure comprise an antigen-binding domain of LC-modified variant 40206, having heavy chain CDR amino acid sequences (HCDR1, HCDR2 and HCDR3) comprising the sequences as set forth in SEQ ID NOs: 6, 7 and 8, and light chain CDR amino acid sequences (LCDR1, LCDR2 and LCDR3) comprising the sequences as set forth in SEQ ID NOs: 71, 19 and 17 as defined by Kabat numbering.

In certain embodiments, the anti-GPC3 antibody constructs of the ADCs of the present disclosure comprise an antigen-binding domain of LC-modified variant 40206 having heavy chain CDR amino acid sequences (HCDR1, HCDR2 and HCDR3) comprising the sequences as set forth in SEQ ID NOs: 3, 4 and 5, and light chain CDR amino acid sequences (LCDR1 and LCDR3) comprising the sequences as set forth in SEQ ID NOs: 70 and 17 and the LCDR2 amino acid sequence KVS, as defined by IMGT numbering.

In certain embodiments, the anti-GPC3 antibody constructs of the ADCs of the present disclosure comprise an antigen-binding domain LC-modified variant 40206 having heavy chain CDR amino acid sequences (HCDR1, HCDR2 and HCDR3) comprising the sequences as set forth in SEQ ID NOs: 9, 10 and 8, and light chain CDR amino acid sequences (LCDR1, LCDR2 and LCDR3) comprising the sequences as set forth in SEQ ID NOs: 71, 19 and 17 as defined by Chothia numbering.

In certain embodiments, the anti-GPC3 antibody constructs of the ADCs of the present disclosure comprise an antigen-binding domain LC-modified variant 40206 having heavy chain CDR amino acid sequences (HCDR1, HCDR2 and HCDR3) comprising the sequences as set forth in SEQ ID NOs: 13, 14 and 15, and light chain CDR amino acid sequences (LCDR1, LCDR2 and LCDR3) comprising the sequences as set forth in SEQ ID NOs: 72, 21 and 22 as defined by Contact numbering.

In certain embodiments, the anti-GPC3 antibody constructs of the ADCs of the present disclosure comprise an antigen-binding domain LC-modified variant 40206 having heavy chain CDR amino acid sequences (HCDR1, HCDR2 and HCDR3) comprising the sequences as set forth in SEQ ID NOs: 11, 12 and 8, and light chain CDR amino acid sequences (LCDR1, LCDR2 and LCDR3) comprising the sequences as set forth in SEQ ID NOs: 71, 19 and 17 as defined by AbM numbering.

In certain embodiments, the anti-GPC3 antibody constructs of the ADCs of the present disclosure comprise an antigen-binding domain of LC-modified variant 40207 having heavy chain CDR amino acid sequences (HCDR1, HCDR2 and HCDR3) comprising the sequences as set forth in SEQ ID NOs: 6, 7 and 8, and light chain CDR amino acid sequences (LCDR1, LCDR2 and LCDR3) comprising the sequences as set forth in SEQ ID NOs: 74, 19 and 17 as defined by Kabat numbering.

In certain embodiments, the anti-GPC3 antibody constructs of the ADCs of the present disclosure comprise an antigen-binding domain of LC-modified variant 40207 having heavy chain CDR amino acid sequences (HCDR1, HCDR2 and HCDR3) comprising the sequences as set forth in SEQ ID NOs: 3, 4 and 5, and light chain CDR amino acid sequences (LCDR1 and LCDR3) comprising the sequences as set forth in SEQ ID NOs: 73 and 17 and the LCDR2 amino acid sequence KVS, as defined by IMGT numbering.

In certain embodiments, the anti-GPC3 antibody constructs of the ADCs of the present disclosure comprise an antigen-binding domain LC-modified variant 40207 having heavy chain CDR amino acid sequences (HCDR1, HCDR2 and HCDR3) comprising the sequences as set forth in SEQ ID NOs: 9, 10 and 8, and light chain CDR amino acid sequences (LCDR1, LCDR2 and LCDR3) comprising the sequences as set forth in SEQ ID NOs: 74, 19 and 17 as defined by Chothia numbering.

In certain embodiments, the anti-GPC3 antibody constructs of the ADCs of the present disclosure comprise an antigen-binding domain LC-modified variant 40207 having heavy chain CDR amino acid sequences (HCDR1, HCDR2 and HCDR3) comprising the sequences as set forth in SEQ ID NOs: 13, 14 and 15, and light chain CDR amino acid sequences (LCDR1, LCDR2 and LCDR3) comprising the sequences as set forth in SEQ ID NOs: 75, 21 and 22 as defined by Contact numbering.

In certain embodiments, the anti-GPC3 antibody constructs of the ADCs of the present disclosure comprise an antigen-binding domain LC-modified variant 40207 having heavy chain CDR amino acid sequences (HCDR1, HCDR2 and HCDR3) comprising the sequences as set forth in SEQ ID NOs: 11, 12 and 8, and light chain CDR amino acid sequences (LCDR1, LCDR2 and LCDR3) comprising the sequences as set forth in SEQ ID NOs: 74, 19 and 17 as defined by AbM numbering.

In certain embodiments, the anti-GPC3 antibody constructs of the ADCs of the present disclosure comprise an antigen-binding domain of LC-modified variant 40208 having heavy chain CDR amino acid sequences (HCDR1, HCDR2 and HCDR3) comprising the sequences as set forth in SEQ ID NOs: 6, 7 and 8, and light chain CDR amino acid sequences (LCDR1, LCDR2 and LCDR3) comprising the sequences as set forth in SEQ ID NOs: 77, 19 and 17 as defined by Kabat numbering.

In certain embodiments, the anti-GPC3 antibody constructs of the ADCs of the present disclosure comprise an antigen-binding domain of LC-modified variant 40208 having heavy chain CDR amino acid sequences (HCDR1, HCDR2 and HCDR3) comprising the sequences as set forth in SEQ ID NOs: 3, 4 and 5, and light chain CDR amino acid sequences (LCDR1 and LCDR3) comprising the sequences as set forth in SEQ ID NOs: 76 and 17 and the LCDR2 amino acid sequence KVS, as defined by IMGT numbering.

In certain embodiments, the anti-GPC3 antibody constructs of the ADCs of the present disclosure comprise an antigen-binding domain LC-modified variant 40208 having heavy chain CDR amino acid sequences (HCDR1, HCDR2 and HCDR3) comprising the sequences as set forth in SEQ ID NOs: 9, 10 and 8, and light chain CDR amino acid sequences (LCDR1, LCDR2 and LCDR3) comprising the sequences as set forth in SEQ ID NOs: 77, 19 and 17 as defined by Chothia numbering.

In certain embodiments, the anti-GPC3 antibody constructs of the ADCs of the present disclosure comprise an antigen-binding domain LC-modified variant 40208 having heavy chain CDR amino acid sequences (HCDR1, HCDR2 and HCDR3) comprising the sequences as set forth in SEQ ID NOs: 13, 14 and 15, and light chain CDR amino acid sequences (LCDR1, LCDR2 and LCDR3) comprising the sequences as set forth in SEQ ID NOs: 78, 21 and 22 as defined by Contact numbering.

In certain embodiments, the anti-GPC3 antibody constructs of the ADCs of the present disclosure comprise an antigen-binding domain LC-modified variant 40208 having heavy chain CDR amino acid sequences (HCDR1, HCDR2 and HCDR3) comprising the sequences as set forth in SEQ ID NOs: 11, 12 and 8, and light chain CDR amino acid sequences (LCDR1, LCDR2 and LCDR3) comprising the sequences as set forth in SEQ ID NOs: 77, 19 and 17 as defined by AbM numbering.

In certain embodiments, the anti-GPC3 antibody constructs of the ADCs of the present disclosure comprise an antigen-binding domain comprising a VH sequence that is at least 80%, at least 85%, at least 90%, at least 91%, at least 92%, at least 93%, at least 94%, at least 95%, at least 96%, at least 97%, at least 98%, at least 99%, or 100% identical to the VH amino acid sequence of v36180 (M3-H1L1) and a VL sequence that is at least 80%, at least 85%, at least 90%, at least 91%, at least 92%, at least 93%, at least 94%, at least 95%, at least 96%, at least 97%, at least 98%, at least 99%, or 100% identical to the VL amino acid sequence of v36180 (M3-H1L1), where the antigen-binding domain retains the ability to bind hGPC3.

In certain embodiments, the anti-GPC3 antibody constructs of the ADCs of the present disclosure comprise an antigen-binding domain comprising a VH sequence having the 3 HCDRs of v36180 (M3-H1L1) and having at least 80%, at least 85%, at least 90%, at least 91%, at least 92%, at least 93%, at least 94%, at least 95%, at least 96%, at least 97%, at least 98%, at least 99%, or 100% identical to the VH amino acid sequence of v36180 (M3-H1L1) and a VL sequence having the 3 LCDRs of v36180 (M3-H1L1) and having at least 80%, at least 85%, at least 90%, at least 91%, at least 92%, at least 93%, at least 94%, at least 95%, at least 96%, at least 97%, at least 98%, at least 99%, or 100% identical to the VL amino acid sequence of v36180 (M3-H1L1), wherein the 3 HCDRs and the 3 LCDRs are defined by IMGT, Kabat, Chothia or AbM numbering systems, where the antigen-binding domain retains the ability to bind hGPC3.

In other embodiments, the anti-GPC3 antibody constructs of the ADCs of the present disclosure comprise an antigen-binding domain comprising a VH sequence that is at least 80%, at least 85%, at least 90%, at least 91%, at least 92%, at least 93%, at least 94%, at least 95%, at least 96%, at least 97%, at least 98%, at least 99%, or 100% identical to the VH amino acid sequence of v37574 (M3-H18L6) and a VL sequence that is at least 80%, at least 85%, at least 90%, at least 91%, at least 92%, at least 93%, at least 94%, at least 95%, at least 96%, at least 97%, at least 98%, at least 99%, or 100% identical to the VL amino acid sequence of v37574 (M3-H18L6), where the antigen-binding domain retains the ability to bind hGPC3.

In other embodiments, the anti-GPC3 antibody constructs of the ADCs of the present disclosure comprise an antigen-binding domain comprising a VH sequence having the 3 HCDRs of v37574 (M3-H18L6) and having at least 80%, at least 85%, at least 90%, at least 91%, at least 92%, at least 93%, at least 94%, at least 95%, at least 96%, at least 97%, at least 98%, at least 99%, or 100% sequence identity to the VH amino acid sequence of v37574 (M3-H18L6) and a VL sequence having the 3 LCDRs of v37574 (M3-H18L6) and having at least 80%, at least 85%, at least 90%, at least 91%, at least 92%, at least 93%, at least 94%, at least 95%, at least 96%, at least 97%, at least 98%, at least 99%, or 100% sequence identity to the VL amino acid sequence of v37574 (M3-H18L6), where the antigen-binding domain retains the ability to bind hGPC3.

In certain embodiments, the anti-GPC3 antibody constructs of the ADCs of the present disclosure comprise an antigen-binding domain comprising a VH sequence that is at least 80%, at least 85%, at least 90%, at least 91%, at least 92%, at least 93%, at least 94%, at least 95%, at least 96%, at least 97%, at least 98%, at least 99%, or 100% identical to the VH amino acid sequence of LC-modified variant 40206 and a VL sequence that is at least 80%, at least 85%, at least 90%, at least 91%, at least 92%, at least 93%, at least 94%, at least 95%, at least 96%, at least 97%, at least 98%, at least 99%, or 100% identical to the VL amino acid sequence of LC-modified variant 40206, where the antigen-binding domain retains the ability to bind hGPC3.

In certain embodiments, the anti-GPC3 antibody constructs of the ADCs of the present disclosure comprise an antigen-binding domain comprising a VH sequence having the 3 HCDRs of LC-modified variant 40206 and having at least 80%, at least 85%, at least 90%, at least 91%, at least 92%, at least 93%, at least 94%, at least 95%, at least 96%, at least 97%, at least 98%, at least 99%, or 100% identical to the VH amino acid sequence of LC-modified variant 40206 and a VL sequence having the 3 LCDRs of LC-modified variant 40206 and having at least 80%, at least 85%, at least 90%, at least 91%, at least 92%, at least 93%, at least 94%, at least 95%, at least 96%, at least 97%, at least 98%, at least 99%, or 100% identical to the VL amino acid sequence of LC-modified variant 40206, wherein the 3 HCDRs and the 3 LCDRs are defined by IMGT, Kabat, Chothia or AbM numbering systems, where the antigen-binding domain retains the ability to bind hGPC3.

In certain embodiments, the anti-GPC3 antibody constructs of the ADCs of the present disclosure comprise an antigen-binding domain comprising a VH sequence that is at least 80%, at least 85%, at least 90%, at least 91%, at least 92%, at least 93%, at least 94%, at least 95%, at least 96%, at least 97%, at least 98%, at least 99%, or 100% identical to the VH amino acid sequence of LC-modified variant 40207 and a VL sequence that is at least 80%, at least 85%, at least 90%, at least 91%, at least 92%, at least 93%, at least 94%, at least 95%, at least 96%, at least 97%, at least 98%, at least 99%, or 100% identical to the VL amino acid sequence of LC-modified variant 40207, where the antigen-binding domain retains the ability to bind hGPC3.

In certain embodiments, the anti-GPC3 antibody constructs of the ADCs of the present disclosure comprise an antigen-binding domain comprising a VH sequence having the 3 HCDRs of LC-modified variant 40207 and having at least 80%, at least 85%, at least 90%, at least 91%, at least 92%, at least 93%, at least 94%, at least 95%, at least 96%, at least 97%, at least 98%, at least 99%, or 100% identical to the VH amino acid sequence of LC-modified variant 40207 and a VL sequence having the 3 LCDRs of LC-modified variant 40207 and having at least 80%, at least 85%, at least 90%, at least 91%, at least 92%, at least 93%, at least 94%, at least 95%, at least 96%, at least 97%, at least 98%, at least 99%, or 100% identical to the VL amino acid sequence of LC-modified variant 40207, wherein the 3 HCDRs and the 3 LCDRs are defined by IMGT, Kabat, Chothia or AbM numbering systems, where the antigen-binding domain retains the ability to bind hGPC3.

In certain embodiments, the anti-GPC3 antibody constructs of the ADCs of the present disclosure comprise an antigen-binding domain comprising a VH sequence that is at least 80%, at least 85%, at least 90%, at least 91%, at least 92%, at least 93%, at least 94%, at least 95%, at least 96%, at least 97%, at least 98%, at least 99%, or 100% identical to the VH amino acid sequence of LC-modified variant 40208 and a VL sequence that is at least 80%, at least 85%, at least 90%, at least 91%, at least 92%, at least 93%, at least 94%, at least 95%, at least 96%, at least 97%, at least 98%, at least 99%, or 100% identical to the VL amino acid sequence of LC-modified variant 40208, where the antigen-binding domain retains the ability to bind hGPC3.

In certain embodiments, the anti-GPC3 antibody constructs of the ADCs of the present disclosure comprise an antigen-binding domain comprising a VH sequence having the 3 HCDRs of LC-modified variant 40208 and having at least 80%, at least 85%, at least 90%, at least 91%, at least 92%, at least 93%, at least 94%, at least 95%, at least 96%, at least 97%, at least 98%, at least 99%, or 100% identical to the VH amino acid sequence of LC-modified variant 40208 and a VL sequence having the 3 LCDRs of LC-modified variant 40208 and having at least 80%, at least 85%, at least 90%, at least 91%, at least 92%, at least 93%, at least 94%, at least 95%, at least 96%, at least 97%, at least 98%, at least 99%, or 100% identical to the VL amino acid sequence of LC-modified variant 40208, wherein the 3 HCDRs and the 3 LCDRs are defined by IMGT, Kabat, Chothia or AbM numbering systems, where the antigen-binding domain retains the ability to bind hGPC3.

In certain embodiments, the anti-GPC3 antibody construct of the ADC of the present disclosure comprises an antigen-binding domain comprising a VH amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to the sequence as set forth in SEQ ID NO: 27, and a VL amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to the sequence as set forth in SEQ ID NO: 28.

In certain embodiments, the anti-GPC3 antibody construct of the ADC of the present disclosure comprises an antigen-binding domain comprising a VH amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to the sequence as set forth in SEQ ID NO: 29, and a VL amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to the sequence as set forth in SEQ ID NO: 30.

In certain embodiments, the anti-GPC3 antibody construct of the ADC of the present disclosure comprises an antigen-binding domain comprising a VH amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to the sequence as set forth in SEQ ID NO: 29, and a VL amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to the sequence as set forth in SEQ ID NO: 68.

In certain embodiments, the anti-GPC3 antibody construct of the ADC of the present disclosure comprises an antigen-binding domain comprising a VH amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to the sequence as set forth in SEQ ID NO: 29, and a VL amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to the sequence as set forth in SEQ ID NO: 64.

In certain embodiments, the anti-GPC3 antibody construct of the ADC of the present disclosure comprises an antigen-binding domain comprising a VH amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to the sequence as set forth in SEQ ID NO: 29, and a VL amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to the sequence as set forth in SEQ ID NO: 60.

In certain embodiments, the anti-GPC3 antibody construct of the ADC of the present disclosure comprises an antigen-binding domain comprising (i) a VH amino acid sequence as set forth in SEQ ID NO: 27, and a VL amino acid sequence as set forth in SEQ ID NO: 28, or (ii) a VH amino acid sequence as set forth in SEQ ID NO: 29, and a VL amino acid sequence as set forth in SEQ ID NO: 30. In other embodiments, the anti-GPC3 antibody construct of the ADC of the present disclosure comprises an antigen-binding domain comprising a VH amino acid sequence as set forth in SEQ ID NO: 29, and a VL amino acid sequence as set forth in SEQ ID NO: 68. In certain embodiments, the anti-GPC3 antibody construct of the ADC of the present disclosure comprises an antigen-binding domain comprising a VH amino acid sequence as set forth in SEQ ID NO: 29, and a VL amino acid sequence as set forth in SEQ ID NO: 64. In still other embodiments, the anti-GPC3 antibody construct of the ADC of the present disclosure comprises an antigen-binding domain comprising a VH amino acid sequence as set forth in SEQ ID NO: 29, and a VL amino acid sequence as set forth in SEQ ID NO: 60.

Exemplary VH and VL sequences are provided in the Examples and Sequence Tables.

In one embodiment the anti-GPC3 construct of the ADCs of the present disclosure comprises two heavy chains comprising the sequence as set forth in SEQ ID NO:50 and two light chains comprising the sequence as set forth in SEQ ID NO:53 (v37574 M3-H18L6). In one embodiment the anti-GPC3 construct of the ADCs of the present disclosure comprises two heavy chains comprising the sequence as set forth in SEQ ID NO:56 and two light chains comprising the sequence as set forth in SEQ ID NO:53 (v38592). In one embodiment the anti-GPC3 construct of the ADCs of the present disclosure comprises two heavy chains comprising the sequence as set forth in SEQ ID NO:44 and two light chains comprising the sequence as set forth in SEQ ID NO:47 (v36180 M3-H1L1). In one embodiment the anti-GPC3 construct of the ADCs of the present disclosure comprises two heavy chains comprising the sequence as set forth in SEQ ID NO:50 and two light chains comprising the sequence as set forth in SEQ ID NO:66 (v40206). In one embodiment the anti-GPC3 construct of the ADCs of the present disclosure comprises two heavy chains comprising the sequence as set forth in SEQ ID NO:50 and two light chains comprising the sequence as set forth in SEQ ID NO:62 (v40207). In one embodiment the anti-GPC3 construct of the ADCs of the present disclosure comprises two heavy chains comprising the sequence as set forth in SEQ ID NO:50 and two light chains comprising the sequence as set forth in SEQ ID NO:58 (v40208).

### Formats

The anti-GPC3 antibody constructs of the ADCs may have various formats. The minimal component of the anti-GPC3 antibody construct is an antigen-binding domain that binds to hGPC3. The anti-GPC3 antibody constructs may further optionally comprise one or more additional antigen-binding domains and/or a scaffold. In those embodiments in which the anti-GPC3 antibody construct comprises two or more antigen-binding domains, each additional antigen-binding domain may bind to the same epitope within hGPC3, may bind to a different epitope within hGPC3, or may bind to a different antigen. Thus, the anti-GPC3 antibody construct may be, for example, monospecific, biparatopic, bispecific or multispecific.

In certain embodiments, the anti-GPC3 antibody construct comprises at least one antigen-binding domain that binds to hGPC3 and a scaffold, where the antigen-binding domain is operably linked to the scaffold. The term “operably linked,” as used herein, means that the components described are in a relationship permitting them to function in their intended manner. Suitable scaffolds are described below.

In certain embodiments, the anti-GPC3 antibody construct comprises two antigen-binding domains optionally operably linked to a scaffold. In some embodiments, the anti-GPC3 antibody construct may comprise three or four antigen-binding domains and optionally a scaffold. In these formats, when comprising a scaffold, at least a first antigen-binding domain is operably linked to the scaffold and the remaining antigen-binding domain(s) may each independently be operably linked to the scaffold or to the first antigen-binding domain or, when more than two antigen-binding domains are present, to another antigen-binding domain.

Anti-GPC3 antibody constructs that lack a scaffold may comprise a single antigen-binding domain in an appropriate format, such as an sdAb, or they may comprise two or more antigen-binding domains optionally operably linked by one or more linkers. In such anti-GPC3 antibody constructs, the antigen-binding domains may be in the form of scFvs, Fabs, sdAbs, or a combination thereof. For example, using scFvs as the antigen-binding domains, formats such as a tandem scFv ((scFv)₂ or taFv) may be constructed, in which the scFvs are connected together by a flexible linker. scFvs may also be used to construct diabody formats, which comprise two scFvs connected by a short linker (usually about 5 amino acids in length). The restricted length of the linker results in dimerization of the scFvs in a head-to-tail manner. In any of the preceding formats, the scFvs may be further stabilized by inclusion of an interdomain disulfide bond. For example, a disulfide bond may be introduced between VL and VH through introduction of an additional cysteine residue in each chain (for example, at position 44 in VH and 100 in VL) (see, for example, Fitzgerald et al., 1997, Protein Engineering, 10:1221-1225), or a disulfide bond may be introduced between two VHs to provide a construct having a DART format (see, for example, Johnson et al., 2010, J Mol. Biol., 399:436-449).

Similarly, formats comprising two sdAbs, such as VHs or VHHs, connected together through a suitable linker may be employed in some embodiments. Other examples of anti-GPC3 antibody construct formats that lack a scaffold include those based on Fab fragments, for example, Fab2 and F(ab′)₂ formats, in which the Fab fragments are connected through a linker or an IgG hinge region.

Combinations of antigen-binding domains in different forms may also be employed to generate alternative scaffold-less formats. For example, an scFv or a sdAb may be fused to the C-terminus of either or both of the light and heavy chain of a Fab fragment resulting in a bivalent (Fab-scFv/sdAb) construct.

In certain embodiments, the anti-GPC3 antibody construct may be in an antibody format that is based on an immunoglobulin (Ig). This type of format is referred to herein as a full-size antibody format (FSA) or Mab format and includes anti-GPC3 antibody constructs that comprise two Ig heavy chains and two Ig light chains. In certain embodiments, the anti-GPC3 antibody construct may be based on an IgG class immunoglobulin, for example, an IgG1, IgG2, IgG3 or IgG4 immunoglobulin. In some embodiments, the anti-GPC3 antibody construct may be based on an IgG1 immunoglobulin. In the context of the present disclosure, when an anti-GPC3 antibody construct is based on a specified immunoglobulin isotype, it is meant that the anti-GPC3 antibody construct comprises all or a portion of the constant region of the specified immunoglobulin isotype. For example, an anti-GPC3 antibody construct based on a given Ig isotype may comprise at least one antigen-binding domain operably linked to an Ig scaffold, where the scaffold comprises an Fc region from the given isotype and optionally an Ig hinge region from the same or a different isotype. It is to be understood that the anti-GPC3 antibody constructs may also comprise hybrids of isotypes and/or subclasses in some embodiments. It is also to be understood that the Fc region and/or hinge region may optionally be modified to impart one or more desirable functional properties as is known in the art. Thus, in certain embodiments, the anti-GPC3 antibody construct comprises a VH amino acid sequence fused to IgG1 constant domain amino acid sequences (i.e. CH1, CH2, CH3 amino acid sequences) and a VL amino acid sequence fused to kappa or lambda constant amino acid sequences domain (i.e. CL amino acid sequences). Exemplary amino acid sequences are provided in the Examples and Sequence Tables.

In some embodiments, the anti-GPC3 antibody constructs may be derived from two or more immunoglobulins that are from different species, for example, the anti-GPC3 antibody construct may be a chimeric antibody or a humanized antibody. The terms “chimeric antibody” and “humanized antibody” both refer generally to antibodies that combine immunoglobulin regions or domains from more than one species.

A “chimeric antibody” typically comprises at least one variable domain from a non-human antibody, such as a rabbit or rodent (for example, murine) antibody, and at least one constant domain from a human antibody. The human constant domain of a chimeric antibody need not be of the same isotype as the non-human constant domain it replaces. Chimeric antibodies are discussed, for example, in Morrison et al., 1984, Proc. Natl. Acad. Sci. USA, 81:6851-55, and U.S. Pat. No. 4,816,567.

A “humanized antibody” is a type of chimeric antibody that contains minimal sequence derived from a non-human antibody. Generally, humanized antibodies are human immunoglobulins (recipient antibody) in which residues from a hypervariable region of the recipient are replaced by residues from a hypervariable region of a non-human species (donor antibody), such as mouse, rat, rabbit or non-human primate, having the desired specificity and affinity for a target antigen. This technique for creating humanized antibodies is often referred to as “CDR grafting.”

In some instances, additional modifications are made to further refine antibody performance. For example, framework region (FR) residues of the human immunoglobulin are replaced by corresponding non-human residues, or the humanized antibodies may comprise residues that are not found in either the recipient antibody or the donor antibody. In general, a variable domain in a humanized antibody will comprise all or substantially all of the hypervariable regions from a non-human immunoglobulin and all or substantially all of the FRs from a human immunoglobulin sequence. Humanized antibodies are described in more detail in Jones, et al., 1986, Nature, 321:522-525; Riechmann, et al., 1988, Nature, 332:323-329, and Presta, 1992, Curr. Op. Struct. Biol., 2:593-596, for example.

A number of approaches are known in the art for selecting the most appropriate human frameworks in which to graft the non-human CDRs. Early approaches used a limited subset of well-characterised human antibodies, irrespective of the sequence identity to the non-human antibody providing the CDRs (the “fixed frameworks” approach). More recent approaches have employed variable regions with high amino acid sequence identity to the variable regions of the non-human antibody providing the CDRs (“homology matching” or “best-fit” approach). An alternative approach is to select fragments of the framework sequences within each light or heavy chain variable region from several different human antibodies. CDR-grafting may in some cases result in a partial or complete loss of affinity of the grafted molecule for its target antigen. In such cases, affinity can be restored by back-mutating some of the residues of human origin to the corresponding non-human ones. Methods for preparing humanized antibodies by these approaches are well-known in the art (see, for example, Tsurushita & Vasquez, 2004, Humanization of Monoclonal Antibodies, Molecular Biology of B Cells, 533-545, Elsevier Science (USA); Jones et al., 1986, Nature, 321:522-525; Riechmann et al., 1988, Nature, 332:323-329; Presta et al., 1997, Cancer Res, 57(20):4593-4599).

Alternatively, or in addition to, these traditional approaches, more recent technologies may be employed to further reduce the immunogenicity of a CDR-grafted humanized antibody. For example, frameworks based on human germline sequences or consensus sequences may be employed as acceptor human frameworks rather than human frameworks with somatic mutation(s). Another technique that aims to reduce the potential immunogenicity of non-human CDRs is to graft only specificity-determining residues (SDRs). In this approach, only the minimum CDR residues required for antigen-binding activity (the “SDRs”) are grafted into a human germline framework. This method improves the “humanness” (i.e. the similarity to human germline sequence) of the humanized antibody and thus may help reduce the risk of immunogenicity of the variable region. These techniques have been described in various publications (see, for example, Almagro & Fransson, 2008, Front Biosci, 13:1619-1633; Tan, et al., 2002, J Immunol, 169:1119-1125; Hwang, et al., 2005, Methods, 36:35-42; Pelat, et al., 2008, J Mol Biol, 384:1400-1407; Tamura, et al., 2000, J Immunol, 164:1432-1441; Gonzales, et al., 2004, Mol Immunol, 1:863-872, and Kashmiri, et al., 2005, Methods, 36:25-34).

### Scaffolds

In certain embodiments, the anti-GPC3 antibody constructs of the ADC comprise one or more antigen-binding domains operably linked to a scaffold. The antigen-binding domain(s) may be in one or a combination of the forms described above (for example, scFvs, Fabs and/or sdAbs). Examples of suitable scaffolds are described in more detail below and include, but are not limited to, immunoglobulin Fc regions, albumin, albumin analogues and derivatives, heterodimerizing peptides (such as leucine zippers, heterodimer-forming “zipper” peptides derived from Jun and Fos, IgG CH1 and CL domains or barnase-barstar toxins), cytokines, chemokines or growth factors. Other examples include antibodies based on the DOCK-AND-LOCK™ (DNL™) technology developed by IBC Pharmaceuticals, Inc. and Immunomedics, Inc. (see, for example, Chang, et al., 2007, Clin. Cancer Res., 13:5586s-5591s).

A scaffold may be a peptide, polypeptide, polymer, nanoparticle or other chemical entity. Where the scaffold is a polypeptide, each antigen-binding domain of the anti-GPC3 antibody construct may be linked to either the N- or C-terminus of the polypeptide scaffold. Anti-GPC3 antibody construct comprising a polypeptide scaffold in which one or more of the antigen-binding polypeptide constructs are linked to a region other than the N- or C-terminus, for example, via the side chain of an amino acid with or without a linker, are also contemplated in certain embodiments.

In embodiments where the anti-GPC3 antibody construct comprises a scaffold that is a peptide or polypeptide, the antigen-binding domain(s) may be linked to the scaffold by genetic fusion or chemical conjugation. Typically, when the scaffold is a peptide or polypeptide, the antigen-binding domain(s) are linked to the scaffold by genetic fusion. In some embodiments, where the scaffold is a polymer or nanoparticle, the antigen-binding domain(s) may be linked to the scaffold by chemical conjugation.

A number of protein domains are known in the art that comprise selective pairs of two different polypeptides and may be used to form a scaffold. An example is leucine zipper domains such as Fos and Jun that selectively pair together (Kostelny, et al., J Immunol, 148:1547-53 (1992); Wranik, et al., J. Biol. Chem., 287: 43331-43339 (2012)). Other selectively pairing molecular pairs include, for example, the barnase-barstar pair (Deyev, et al., Nat Biotechnol, 21:1486-1492 (2003)), DNA strand pairs (Chaudri, et al., FEBS Letters, 450(1-2):23-26 (1999)) and split fluorescent protein pairs (International Patent Application Publication No. WO 2011/135040).

Other examples of protein scaffolds include immunoglobulin Fc regions, albumin, albumin analogues and derivatives, toxins, cytokines, chemokines and growth factors. The use of protein scaffolds in combination with antigen-binding moieties has been described (see, for example, Müller et al., 2007, J. Biol. Chem., 282:12650-12660; McDonaugh et al., 2012, Mol. Cancer Ther., 11:582-593; Vallera et al., 2005, Clin. Cancer Res., 11:3879-3888; Song et al., 2006, Biotech. Appl. Biochem., 45:147-154, and U.S. Patent Application Publication No. 2009/0285816).

For example, fusing antigen-binding moieties such as scFvs, diabodies or single chain diabodies to albumin has been shown to improve the serum half-life of the antigen-binding moieties (Müller et al., ibid.). Antigen-binding moieties may be fused at the N- and/or C-termini of albumin, optionally via a linker.

Derivatives of albumin in the form of heteromultimers that comprise two transporter polypeptides obtained by segmentation of an albumin protein such that the transporter polypeptides self-assemble to form quasi-native albumin have been described (see International Patent Application Publication Nos. WO 2012/116453 and WO 2014/012082). As a result of the segmentation of albumin, the heteromultimer includes four termini and thus can be fused to up to four different antigen-binding moieties, optionally via linkers.

In certain embodiments, the anti-GPC3 antibody construct of the ADC may comprise a protein scaffold. In some embodiments, the anti-GPC3 antibody construct may comprise a protein scaffold that is based on an immunoglobulin Fc region, an albumin or an albumin analogue or derivative. In some embodiments, the anti-GPC3 antibody construct may comprise a protein scaffold that is based on an immunoglobulin Fc region, for example, an IgG Fc region.

### Fc Regions

The terms “Fc region,” “Fc” or “Fc domain” as used herein refer to a C-terminal region of an immunoglobulin heavy chain that contains at least a portion of the constant region. The term includes native sequence Fc regions and variant Fc regions. Unless otherwise specified herein, numbering of amino acid residues in the Fc region or constant region is according to the EU numbering system, also called the EU index, as described in Kabat, et al., Sequences of Proteins of Immunological Interest, 5th Ed. Public Health Service, National Institutes of Health, Bethesda, MD (1991).

In certain embodiments, the anti-GPC3 antibody constructs of the ADC may comprise a scaffold that is based on an immunoglobulin Fc region. The Fc region may be dimeric and composed of two Fc polypeptides or alternatively, the Fc region may be composed of a single polypeptide.

An “Fc polypeptide” in the context of a dimeric Fc refers to one of the two polypeptides forming the dimeric Fc domain, i.e. a polypeptide comprising one or more C-terminal constant regions of an immunoglobulin heavy chain that is capable of stable self-association. When referring to a dimeric Fc region, the terms “first Fc polypeptide” and “second Fc polypeptide” may be used interchangeably provided that the Fc region comprises one first Fc polypeptide and one second Fc polypeptide.

An Fc region may comprise a CH3 domain or it may comprise both a CH3 and a CH2 domain. For example, in certain embodiments, an Fc polypeptide of a dimeric IgG Fc region may comprise an IgG CH2 domain sequence and an IgG CH3 domain sequence. In such embodiments, the CH3 domain comprises two CH3 sequences, one from each of the two Fc polypeptides of the dimeric Fc region, and the CH2 domain comprises two CH2 sequences, one from each of the two Fc polypeptides of the dimeric Fc region.

In some embodiments, the anti-GPC3 antibody construct of the ADC may comprise a scaffold that is based on an IgG Fc region. In some embodiments, the anti-GPC3 antibody construct may comprise a scaffold that is based on a human IgG Fc region. In some embodiments, the anti-GPC3 antibody construct may comprise a scaffold based on an IgG1 Fc region. In some embodiments, the anti-GPC3 antibody construct may comprise a scaffold based on a human IgGI Fc region.

In certain embodiments, the anti-GPC3 antibody construct may comprise a scaffold based on an IgG Fc region, which is a heterodimeric Fc region, comprising a first Fc polypeptide and a second Fc polypeptide, each comprising a CH3 sequence, and optionally a CH2 sequence and in which the first and second Fc polypeptides are different. In some embodiments, the anti-GPC3 antibody construct may comprise a scaffold based on an Fc region which comprises two CH3 sequences, at least one of which comprises one or more amino acid modifications. In some embodiments, the anti-GPC3 antibody construct may comprise a scaffold based on an Fc region which comprises two CH3 sequences and two CH2 sequences, at least one of the CH2 sequences comprising one or more amino acid modifications.

In some embodiments, the anti-GPC3 antibody construct may comprise a heterodimeric Fc region comprising a modified CH3 domain, where the modified CH3 domain is an asymmetrically modified CH3 domain comprising one or more asymmetric amino acid modifications. As used herein, an “asymmetric amino acid modification” refers to a modification, such as a substitution or an insertion, in which an amino acid at a specific position on a first CH3 or CH2 sequence is different to the amino acid on a second CH3 or CH2 sequence at the same position. These asymmetric amino acid modifications can be a result of modification of only one of the two amino acids at the same respective amino acid position on each sequence, or different modifications of both amino acids on each sequence at the same respective position on each of the first and second CH3 or CH2 sequences. Each of the first and second CH3 or CH2 sequences of a heterodimeric Fc may comprise one or more than one asymmetric amino acid modification.

In some embodiments, the anti-GPC3 antibody construct may comprise a heterodimeric Fc comprising a modified CH3 domain, where the modified CH3 domain comprises one or more amino acid modifications that promote formation of the heterodimeric Fc over formation of a homodimeric Fc. In some embodiments, one or more of the amino acid modifications are asymmetric amino acid modifications.

Amino acid modifications that may be made to the CH3 domain of an Fc in order to promote formation of a heterodimeric Fc are known in the art and include, for example, those described in International Publication No. WO 96/027011 (“knobs into holes”), Gunasekaran et al., 2010, J Biol Chem, 285, 19637-46 (“electrostatic steering”), Davis et al., 2010, Prot Eng Des Sel, 23(4):195-202 (strand exchange engineered domain (SEED) technology) and Labrijn et al., 2013, Proc Natl Acad Sci USA, 110(13):5145-50 (Fab-arm exchange). Other examples include approaches combining positive and negative design strategies to produce stable asymmetrically modified Fc regions as described in International Publication Nos. WO 2012/058768 and WO 2013/063702. In certain embodiments, the anti-GPC3 antibody construct may comprise a scaffold based on a modified Fc region as described in International Publication No. WO 2012/058768 or WO 2013/063702.

Table 4 provides the amino acid sequence of the human IgG1 Fc sequence (SEQ ID NO:16), corresponding to amino acids 231 to 447 of the full-length human IgG1 heavy chain. The CH3 sequence comprises amino acids 341-447 of the full-length human IgG1 heavy chain. Also shown in Table 4 are CH3 domain amino acid modifications that promote formation of a heterodimeric Fc as described in in International Patent Application Publication Nos. WO 2012/058768 and WO 2013/063702.

In certain embodiments, the anti-GPC3 antibody construct may comprise a heterodimeric Fc scaffold having a modified CH3 domain comprising the modifications of any one of Variant 1, Variant 2, Variant 3, Variant 4 or Variant 5, as shown in Table 4.

TABLE 4
Human IgG1 Fc Sequence¹ and CH3 Domain Amino Acid
Modifications Promoting Heterodimer Formation

APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHE
ALHNHYTQKSLSLSPGK (SEQ ID NO: 23)

| Variant No | Chain | Mutations |
|---|---|---|
| 1 | A | L351Y_F405A_Y407V |
| | B | T366L_K392M_T394W |
| 2 | A | L351Y_F405A_Y407V |
| | B | T366L_K392L_T394W |
| 3 | A | T350V_L351Y_F405A_Y407V |
| | B | T350V_T366L_K392L_T394W |
| 4 | A | T350V_L351Y_F405A_Y407V |
| | B | T350V_T366L_K392M_T394W |
| 5 | A | T350V_L351Y_S400E_F405A_Y407V |
| | B | T350V_T366L_N390R_K392M_T394W |

¹Sequence from positions 231-447 (EU numbering)

In some embodiments, the anti-GPC3 antibody construct may comprise a scaffold based on an Fc region comprising two CH3 sequences and two CH2 sequences, at least one of the CH2 sequences comprising one or more amino acid modifications. Modifications in the CH2 domain can affect the binding of Fc receptors (FcRs) to the Fc, such as receptors of the FcγRI, FcγRII and FcγRIII subclasses.

In some embodiments, the anti-GPC3 antibody construct comprises a scaffold based on an IgG Fe having a modified CH2 domain, wherein the modification of the CH2 domain results in altered binding to one or more of the FcγRI, FcγRII and FcγRIII receptors.

A number of amino acid modifications to the CH2 domain that selectively alter the affinity of the Fc for different Fcγ receptors are known in the art. Amino acid modifications that result in increased binding and amino acid modifications that result in decreased binding can each be useful in certain indications. For example, increasing binding affinity of an Fc for FcγRIIIa (an activating receptor) may result in increased antibody dependent cell-mediated cytotoxicity (ADCC), which in turn results in increased lysis of the target cell. Decreased binding to FcγRIIb (an inhibitory receptor) likewise may be beneficial in some circumstances. In certain indications, a decrease in, or elimination of, ADCC and complement-mediated cytotoxicity (CDC) may be desirable. In such cases, modified CH2 domains comprising amino acid modifications that result in increased binding to FcγRIIb or amino acid modifications that decrease or eliminate binding of the Fc region to all of the Fcγ receptors (“knock-out” variants) may be useful.

Examples of amino acid modifications to the CH2 domain that alter binding of the Fc by Fcγ receptors include, but are not limited to, the following: S298A/E333A/K334A and S298A/E333A/K334A/K326A (increased affinity for FcγRIIIa) (Lu, et al., 2011, J Immunol Methods, 365(1-2):132-41); F243L/R292P/Y300L/V305I/P396L (increased affinity for FcγRIIIa) (Stavenhagen, et al., 2007, Cancer Res, 67(18):8882-90); F243L/R292P/Y300L/L235V/P396L (increased affinity for FcγRIIIa) (Nordstrom J L, et al., 2011, Breast Cancer Res, 13(6): R¹²³); F243L (increased affinity for FcγRIIIa) (Stewart, et al., 2011, Protein Eng Des Sel., 24(9):671-8); S298A/E333A/K334A (increased affinity for FcγRIIIa) (Shields, et al., 2001, J Biol Chem, 276(9):6591-604); S239D/I332E/A330L and S239D/I332E (increased affinity for FcγRIIIa) (Lazar, et al., 2006, Proc Natl Acad Sci USA, 103(11):4005-10), and S239D/S267E and S267E/L328F (increased affinity for FcγRIIb) (Chu, et al., 2008, Mol Immunol, 45(15):3926-33). Various amino acid modifications to the CH2 domain that alter binding of the Fc by FcγRIIb are described in International Publication No. WO 2021/232162. Additional modifications that affect Fc binding to Fcγ receptors are described in Therapeutic Antibody Engineering (Strohl & Strohl, Woodhead Publishing series in Biomedicine No 11, ISBN 1 907568 37 9, October 2012, page 283).

In certain embodiments, the anti-GPC3 antibody construct comprises a scaffold based on an IgG Fe having a modified CH2 domain, in which the modified CH2 domain comprises one or more amino acid modifications that result in decreased or eliminated binding of the Fc region to all of the Fcγ receptors (i.e. a “knock-out” variant).

Various publications describe strategies that have been used to engineer antibodies to produce “knock-out” variants (see, for example, Strohl, 2009, Curr Opin Biotech 20:685-691, and Strohl & Strohl, “Antibody Fc engineering for optimal antibody performance” In Therapeutic Antibody Engineering, Cambridge: Woodhead Publishing, 2012, pp 225-249). These strategies include reduction of effector function through modification of glycosylation, use of IgG2/IgG4 scaffolds, or the introduction of mutations in the hinge or CH2 domain of the Fc (see also, U.S. Patent Publication No. 2011/0212087, International Publication No. WO 2006/105338, U.S. Patent Publication No. 2012/0225058, U.S. Patent Publication No. 2012/0251531 and Strop et al., 2012, J. Mol. Biol., 420: 204-219).

Examples of mutations that may be introduced into the hinge or CH2 domain to produce a “knock-out” variant include the amino acid modifications L234A/L235A, and L234A/L235A/D265S.

In certain embodiments, the anti-GPC3 antibody constructs described herein may comprise a scaffold based on an IgG Fc in which native glycosylation has been modified. As is known in the art, glycosylation of an Fc may be modified to increase or decrease effector function. For example, mutation of the conserved asparagine residue at position 297 to alanine, glutamine, lysine or histidine (i.e. N297A, Q, K or H) results in an aglycoslated Fc that lacks all effector function (Bolt et al., 1993, Eur. J Immunol., 23:403-411; Tao & Morrison, 1989, J. Immunol., 143:2595-2601).

Conversely, removal of fucose from heavy chain N297-linked oligosaccharides has been shown to enhance ADCC, based on improved binding to FcγRIIIa (see, for example, Shields et al., 2002, J Biol Chem., 277:26733-26740, and Niwa et al., 2005, J. Immunol. Methods, 306:151-160). Such low fucose antibodies may be produced, for example in knockout Chinese hamster ovary (CHO) cells lacking fucosyltransferase (FUT8) (Yamane-Ohnuki et al., 2004, Biotechnol. Bioeng., 87:614-622); in the variant CHO cell line, Lec 13, that has a reduced ability to attach fucose to N297-linked carbohydrates (International Publication No. WO 03/035835), or in other cells that generate afucosylated antibodies (see, for example, Li et al., 2006, Nat Biotechnol, 24:210-215; Shields et al., 2002, ibid, and Shinkawa et al., 2003, J. Biol. Chem., 278:3466-3473). In addition, International Publication No. WO 2009/135181 describes the addition of fucose analogues to culture medium during antibody production to inhibit incorporation of fucose into the carbohydrate on the antibody.

Other methods of producing antibodies with little or no fucose on the Fc glycosylation site (N297) are well known in the art. For example, the GlymaX@technology (ProBioGen AG) (see von Horsten et al., 2010, Glycobiology, 20(12):1607-1618 and U.S. Pat. No. 8,409,572).

Other glycosylation variants include those with bisected oligosaccharides, for example, variants in which a biantennary oligosaccharide attached to the Fc region of the antibody is bisected by N-acetylglucosamine (GlcNAc). Such glycosylation variants may have reduced fucosylation and/or improved ADCC function (see, for example, International Publication No. WO 2003/011878, U.S. Pat. No. 6,602,684 and US Patent Application Publication No. US 2005/0123546). Useful glycosylation variants also include those having at least one galactose residue in the oligosaccharide attached to the Fc region, which may have improved CDC function (see, for example, International Publication Nos. WO 1997/030087, WO 1998/58964 and WO 1999/22764).

### Preparation of Anti-GPC3 Antibody Constructs

The anti-GPC3 antibody constructs described herein may be produced using standard recombinant methods known in the art (see, for example, U.S. Pat. No. 4,816,567 and “Antibodies: A Laboratory Manual,” 2^{nd} Edition, Ed. Greenfield, Cold Spring Harbor Laboratory Press, New York, 2014).

Typically, for recombinant production of an antibody construct, a polynucleotide or set of polynucleotides encoding the anti-GPC3 antibody construct is generated and inserted into one or more vectors for further cloning and/or expression in a host cell. Polynucleotide(s) encoding the anti-GPC3 antibody construct may be produced by standard methods known in the art (see, for example, Ausubel et al., Current Protocols in Molecular Biology, John Wiley & Sons, New York, 1994 & update, and “Antibodies: A Laboratory Manual,” 2^{nd} Edition, Ed. Greenfield, Cold Spring Harbor Laboratory Press, New York, 2014). As would be appreciated by one of skill in the art, the number of polynucleotides required for expression of the anti-GPC3 antibody construct will be dependent on the format of the construct, including whether or not the antibody construct comprises a scaffold. For example, when an anti-GPC3 antibody construct is in a monospecific mAb or FSA format, two polynucleotides one encoding a light chain polypeptide and one encoding a heavy chain polypeptide will be required. When multiple polynucleotides are required, they may be incorporated into one vector or into more than one vector.

Generally, for expression, the polynucleotide or set of polynucleotides is incorporated into an expression vector or vectors together with one or more regulatory elements, such as transcriptional elements, which are required for efficient transcription of the polynucleotide. Examples of such regulatory elements include, but are not limited to, promoters, enhancers, terminators, and polyadenylation signals. One skilled in the art will appreciate that the choice of regulatory elements is dependent on the host cell selected for expression of the antibody construct and that such regulatory elements may be derived from a variety of sources, including bacterial, fungal, viral, mammalian or insect genes. The expression vector may optionally further contain heterologous nucleic acid sequences that facilitate expression or purification of the expressed protein. Examples include, but are not limited to, signal peptides and affinity tags such as metal-affinity tags, histidine tags, avidin/streptavidin encoding sequences, glutathione-S-transferase (GST) encoding sequences and biotin encoding sequences. The expression vector may be an extrachromosomal vector or an integrating vector.

Suitable host cells for cloning or expression of the anti-GPC3 antibody constructs include various prokaryotic or eukaryotic cells as known in the art. Eukaryotic host cells include, for example, mammalian cells, plant cells, insect cells and yeast cells (such as Saccharomyces or Pichia cells). Prokaryotic host cells include, for example, E. coli, A. salmonicida or B. subtilis cells.

In certain embodiments, the anti-GPC3 antibody construct may be produced in bacteria, in particular when glycosylation and Fc effector function are not needed, as described for example in U.S. Pat. Nos. 5,648,237; 5,789,199, and 5,840,523, and in Charlton, Methods in Molecular Biology, Vol. 248, pp. 245-254, B. K. C. Lo, ed., Humana Press, Totowa, N.J., 2003.

Eukaryotic microbes such as filamentous fungi or yeast may be suitable expression host cells in certain embodiments, in particular fungi and yeast strains whose glycosylation pathways have been “humanized” resulting in the production of an antibody construct with a partially or fully human glycosylation pattern (see, for example, Gerngross, 2004, Nat. Biotech. 22:1409-1414, and Li et al., 2006, Nat. Biotech. 24:210-215).

Suitable host cells for the expression of glycosylated anti-GPC3 antibody constructs are usually eukaryotic cells. For example, U.S. Pat. Nos. 5,959,177, 6,040,498, 6,420,548, 7,125,978 and 6,417,429 describe PLANTIBODIES™ technology for producing antigen-binding constructs in transgenic plants. Mammalian cell lines adapted to grow in suspension may be particularly useful for expression of antibody constructs. Examples include, but are not limited to, monkey kidney CV1 line transformed by SV40 (COS-7), human embryonic kidney (HEK) line 293 or 293 cells (see, for example, Graham et al., 1977, J. Gen Virol., 36:59), baby hamster kidney cells (BHK), mouse sertoli TM4 cells (see, for example, Mather, 1980, Biol Reprod, 23:243-251), monkey kidney cells (CV1), African green monkey kidney cells (VERO-76), human cervical carcinoma (HeLa) cells, canine kidney cells (MDCK), buffalo rat liver cells (BRL 3A), human lung cells (W138), human liver cells (Hep G2), mouse mammary tumour (MMT 060562), TRI cells (see, for example, Mather et al., 1982, Annals N.Y. Acad Sci, 383:44-68), MRC 5 cells, FS4 cells, Chinese hamster ovary (CHO) cells (including DHFR⁻ CHO cells, see Urlaub et al., 1980, Proc Natl Acad Sci USA, 77:4216), and myeloma cell lines (such as Y0, NS0 and Sp2/0). Exemplary mammalian host cell lines suitable for production of antibody constructs are reviewed in Yazaki & Wu, Methods in Molecular Biology, Vol. 248, pp. 255-268 (B. K. C. Lo, ed., Humana Press, Totowa, N.J., 2003).

In certain embodiments, the host cell may be a transient or stable higher eukaryotic cell line, such as a mammalian cell line. In some embodiments, the host cell may be a mammalian HEK293T, CHO, HeLa, NS0 or COS cell line, or a cell line derived from any one of these cell lines. In some embodiments, the host cell may be a stable cell line that allows for mature glycosylation of the antibody construct.

The host cells comprising the expression vector(s) encoding the anti-GPC3 antibody construct may be cultured using routine methods to produce the anti-GPC3 antibody construct. Alternatively, in some embodiments, host cells comprising the expression vector(s) encoding the anti-GPC3 antibody construct may be used therapeutically or prophylactically to deliver the anti-GPC3 antibody construct to a subject, or polynucleotides or expression vectors may be administered to a cell from a subject ex vivo and the cell then returned to the body of the subject.

Typically, the anti-GPC3 antibody constructs are purified after expression. Proteins may be isolated or purified in a variety of ways known to those skilled in the art (see, for example, Protein Purification: Principles and Practice, 3^{rd} Ed., Scopes, Springer-Verlag, NY, 1994). Standard purification methods include chromatographic techniques, including ion exchange, hydrophobic interaction, affinity, sizing or gel filtration, and reverse-phase, carried out at atmospheric pressure or at high pressure using systems such as FPLC and HPLC. Additional purification methods include electrophoretic, immunological, precipitation, dialysis and chromatofocusing techniques. Ultrafiltration and diafiltration techniques, in conjunction with protein concentration, are also useful. As is well known in the art, a variety of natural proteins bind Fc and antibodies, and these proteins may be used for purification of certain antibody constructs. For example, the bacterial proteins A and G bind to the Fc region. Likewise, the bacterial protein L binds to the Fab region of some antibodies. Purification may also be enabled by a particular fusion partner. For example, antibodies may be purified using glutathione resin if a GST fusion is employed, Ni⁺² affinity chromatography if a His-tag is employed or immobilized anti-flag antibody if a flag-tag is used. The degree of purification necessary will vary depending on the use of the anti-GPC3 antibody constructs. In some instances, no purification may be necessary.

In certain embodiments, the anti-GPC3 antibody constructs are substantially pure. The term “substantially pure” (or “substantially purified”) when used in reference to an anti-GPC3 antibody construct described herein, means that the antibody construct is substantially or essentially free of components that normally accompany or interact with the protein as found in its naturally occurring environment, such as a native cell, or a host cell in the case of recombinantly produced construct. In certain embodiments, an anti-GPC3 antibody construct that is substantially pure is a protein preparation having less than about 30%, less than about 25%, less than about 20%, less than about 15%, less than about 10%, or less than about 5% (by dry weight) of contaminating protein.

Certain embodiments of the present disclosure relate to a method of making an anti-GPC3 antibody construct comprising culturing a host cell into which one or more polynucleotides encoding the anti-GPC3 antibody construct, or one or more expression vectors encoding the anti-GPC3 antibody construct, have been introduced, under conditions suitable for expression of the anti-GPC3 antibody construct, and optionally recovering the anti-GPC3 antibody construct from the host cell (or from host cell culture medium).

### Post-Translational Modifications

In certain embodiments, the anti-GPC3 antibody constructs described herein may comprise one or more post-translational modifications. Such post-translational modifications may occur in vivo, or they be conducted in vitro after isolation of the anti-GPC3 antibody construct from the host cell.

Post-translational modifications include various modifications as are known in the art (see, for example, Proteins—Structure and Molecular Properties, 2nd Ed., T. E. Creighton, W. H. Freeman and Company, New York, 1993; Post-Translational Covalent Modification of Proteins, B. C. Johnson, Ed., Academic Press, New York, pgs. 1-12, 1983; Seifter et al., 1990, Meth. Enzymol., 182:626-646, and Rattan et al., 1992, Ann. N.Y. Acad. Sci., 663:48-62). In those embodiments in which the anti-GPC3 antibody constructs comprise one or more post-translational modifications, the constructs may comprise the same type of modification at one or several sites, or it may comprise different modifications at different sites.

Examples of post-translational modifications include glycosylation, acetylation, phosphorylation, amidation, derivatization by known protecting/blocking groups, formylation, oxidation, reduction, proteolytic cleavage or specific chemical cleavage by cyanogen bromide, trypsin, chymotrypsin, papain, V8 protease or NaBH4.

Other examples of post-translational modifications include, for example, addition or removal of N-linked or O-linked carbohydrate chains, chemical modifications of N-linked or O-linked carbohydrate chains, processing of N-terminal or C-terminal ends, attachment of chemical moieties to the amino acid backbone, and addition or deletion of an N-terminal methionine residue resulting from prokaryotic host cell expression. Post-translational modifications may also include modification with a detectable label, such as an enzymatic, fluorescent, luminescent, isotopic or affinity label to allow for detection and isolation of the protein. Examples of suitable enzyme labels include, but are not limited to, horseradish peroxidase, alkaline phosphatase, beta-galactosidase and acetylcholinesterase. Examples of suitable prosthetic group complexes include, but are not limited to, streptavidin/biotin and avidin/biotin. Examples of suitable fluorescent materials include, but are not limited to, umbelliferone, fluorescein, fluorescein isothiocyanate, rhodamine, dichlorotriazinylamine fluorescein, dansyl chloride and phycoerythrin. Examples of luminescent materials include luminol, and bioluminescent materials such as luciferase, luciferin and aequorin. Examples of suitable radioactive materials include iodine, carbon, sulfur, tritium, indium, technetium, thallium, gallium, palladium, molybdenum, xenon and fluorine.

Additional examples of post-translational modifications include acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, gamma-carboxylation, GPI anchor formation, hydroxylation, iodination, methylation, myristylation, pegylation, prenylation, racemization, selenoylation, sulfation, transfer-RNA mediated addition of amino acids to proteins such as arginylation, and ubiquitination.

### Camptothecin Analogues

The camptothecin analogue comprised by the ADCs of the present disclosure is a compound having Formula (I):

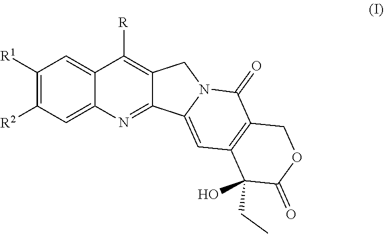

wherein:
   R¹ is selected from: —H, —CH₃, —CHF₂, —CF₃, —F, —Br, —Cl, —OH, —OCH₃, —OCF₃ and —NH₂, and
   R² is selected from: —H, —CH₃, —CF₃, —F, —Br, —Cl, —OH, —OCH₃ and —OCF₃, and wherein:
   when R¹ is-NH₂, then R is R³ or R⁴, and when R¹ is other than-NH₂, then R is R⁴;
   R³ is selected from: —H, —C₁-C₆ alkyl, —C₃-C₈ cycloalkyl, —(C₁-C₆ alkyl)-O—R⁵,

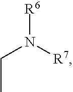

—CO₂R⁸, -aryl, -heteroaryl and —(C₁-C₆ alkyl)-aryl;
   R⁴ is selected from:

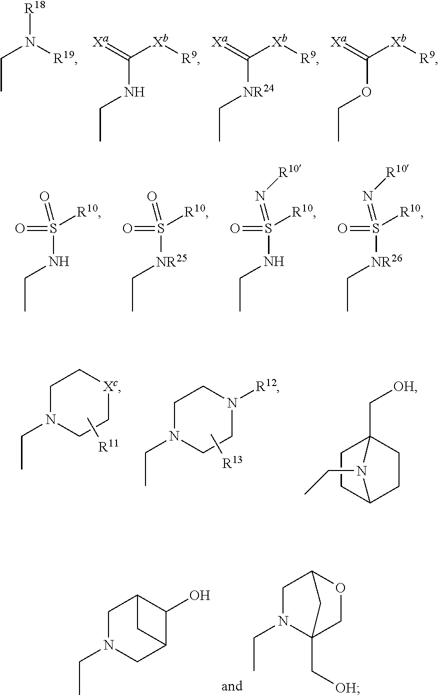

R⁵ is selected from: —H, —C₁-C₆ alkyl, —C₃-C₈ cycloalkyl, -aryl, -heteroaryl, -aryl and —(C₁-C₆ alkyl)-aryl;
R⁶ and R⁷ are each independently selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, —NR¹⁴R^{14′},-aryl, -heteroaryl and —(C₁-C₆ alkyl)-aryl;
R^{10′}is selected from: —H, —C₁-C₆ alkyl, —C₃-C₈ cycloalkyl, -aryl, -heteroaryl, and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl,-S(O)₂R¹⁶ and

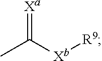

R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹¹ is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R¹⁸ and R¹⁹ taken together with the N atom to which they are bonded form a 4-, 5-, 6- or 7-membered ring having 0 to 3 substituents selected from: halogen, —C₁-C₆ alkyl, —C₃-C₈ cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, 0 and S, and
X^{c} is selected from; 0, S and S(O)₂,
with the proviso that the compound is other than (S)-9-amino-11-butyl-4-ethyl-4-hydroxy-1,12-dihydro-14H-pyrano[3′,4′:6,7]indolizino[1,2-b]quinoline-3,14(4H)-dione.

In some embodiments, the camptothecin analogues are compounds of Formula (I), with the proviso that when R¹ is NH₂, R² is other than H.

In some embodiments, in compounds of Formula (I), R¹ is selected from: —CH₃, —CF₃, —OCH₃, —OCF₃ and NH₂.

In some embodiments, in compounds of Formula (I), R¹ is NH₂.

In some embodiments, in compounds of Formula (I), R¹ is selected from: —H, —CH₃, —CF₃, —F, —Br, —Cl, —OH, —OCH₃ and —OCF₃.

In some embodiments, in compounds of Formula (I), R¹ is selected from: —CH₃, —CF₃, —OCH₃ and —OCF₃.

In some embodiments, in compounds of Formula (I), R² is selected from: —H, —CH₃, —CF₃, —F, —Cl, —OCH₃ and —OCF₃.

In some embodiments, in compounds of Formula (I), R² is selected from: —CH₃, —CF₃, —F, —Cl, —OCH₃ and —OCF₃.

In some embodiments, in compounds of Formula (I), R² is selected from: —H, —F, —Br and-Cl.

In some embodiments, in compounds of Formula (I), R² is selected from: —F, —Br and —Cl.

In some embodiments, in compounds of Formula (I), R³ is selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵,

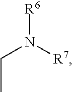

—CO₂R⁸, unsubstituted-aryl, -aminoaryl, -heteroaryl and —(C₁-C₆ alkyl)-aminoaryl.

In some embodiments, in compounds of Formula (I), R⁴ is selected from:

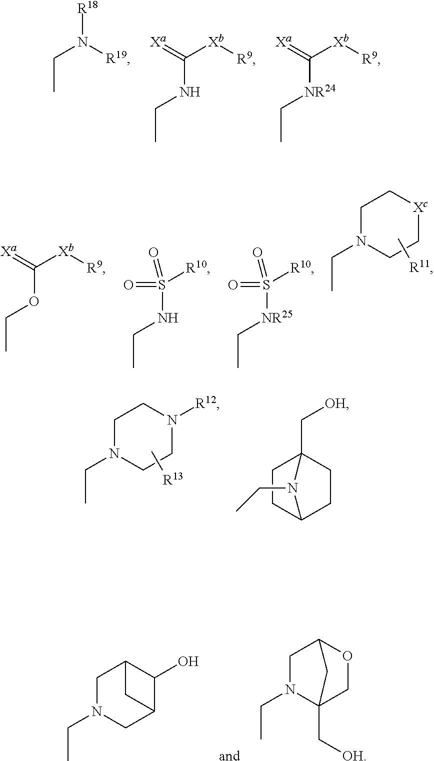

In some embodiments, in compounds of Formula (I), R⁵ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted-aryl, -aminoaryl, -heteroaryl and —(C₁-C₆ alkyl)-aminoaryl.

In some embodiments, in compounds of Formula (I), R⁶ and R⁷ are each independently selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵, —C₃-C₈ heterocycloalkyl and —C(O)R¹⁷.

In some embodiments, in compounds of Formula (I), R⁸ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (I), each R⁹ is independently selected from: —C₁-C₆ alkyl, —C₃-C₈ cycloalkyl, -aryl and —(C₁-C₆ alkyl)-aryl.

In some embodiments, in compounds of Formula (I), each R⁹ is independently selected from: —C₁-C₆ alkyl and —(C₁-C₆ alkyl)-aryl.

In some embodiments, in compounds of Formula (I), each R⁹ is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted-aryl, -aminoaryl, -heteroaryl and —(C₁-C₆ alkyl)-aminoaryl.

In some embodiments, in compounds of Formula (I), each R¹⁰ is independently selected from: —C₁-C₆ alkyl, —C₃-C₈ cycloalkyl, —NR¹⁴R^{14′}, -aryl and —(C₁-C₆ alkyl)-aryl.

In some embodiments, in compounds of Formula (I), each R¹⁰ is independently selected from: —C₁-C₆ alkyl, —NR¹⁴R^{14′}, -aryl and —(C₁-C₆ alkyl)-aryl.

In some embodiments, in compounds of Formula (I), each R¹⁰ is independently selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, —NR¹⁴R^{14′}, unsubstituted-aryl, -aminoaryl, -heteroaryl and —(C₁-C₆ alkyl)-aryl.

In some embodiments, in compounds of Formula (I), R^{10′} is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted-aryl, -aminoaryl, -heteroaryl and —(C₁-C₆ alkyl)-aryl.

In some embodiments, in compounds of Formula (I), R¹¹ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (I), R¹² is selected from: —H, —C₁-C₆ alkyl, —CO₂R⁸, -aryl, —(C₁-C₆ alkyl)-aryl and —S(O)₂R¹⁶.

In some embodiments, in compounds of Formula (I), R¹² is selected from: —H, unsubstituted —C₁-C₆ alkyl, —C₁-C₆ haloalkyl, —C₁-C₆ hydroxyalkyl, —C₁-C₆ aminoalkyl, —CO₂R⁸, unsubstituted-aryl, -aminoaryl, -heteroaryl, —(C₁-C₆ alkyl)-aminoaryl, —S(O)₂R¹⁶ and

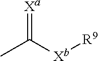

In some embodiments, in compounds of Formula (I), R¹³ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (I), R¹⁴ and R^{14′} are each independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (I), R¹⁶ is selected from: -aryl, -heteroaryl and —(C₁-C₆ alkyl)-aryl.

In some embodiments, in compounds of Formula (I), R¹⁶ is selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted aryl, -aminoaryl, -heteroaryl and —(C₁-C₆ alkyl)-aminoaryl.

In some embodiments, in compounds of Formula (I), R¹¹ is selected from: unsubstituted C₁-C₆ alkyl, —C₁-C₆ hydroxyalkyl, —C₃-C₈ cycloalkyl, —C₃-C₈ heterocycloalkyl, —(C₁-C₆ alkyl)-C₃-C₈ heterocycloalkyl, unsubstituted aryl, -hydroxyaryl, -aminoaryl, -heteroaryl and —(C₁-C₆ alkyl)-aminoaryl.

In some embodiments, in compounds of Formula (I), R¹⁸ and R¹⁹ taken together with the N atom to which they are bonded form a 4-, 5-, 6- or 7-membered ring having 0 to 3 substituents selected from: halogen, unsubstituted C₁-C₆ alkyl, —C₁-C₆ haloalkyl, —C₁-C₆ hydroxyalkyl, —C₁-C₆ aminoalkyl, —C₃-C₈ cycloalkyl and —(C₁-C₆ alkyl)-O—R⁵.

In some embodiments, in compounds of Formula (I), X^{a} and X^{b} are each independently selected from: NH and O.

Combinations of any of the foregoing embodiments for compounds of Formula (I) are also contemplated and each combination forms a separate embodiment for the purposes of the present disclosure.

In certain embodiments, the compound of Formula (I) has Formula (II):

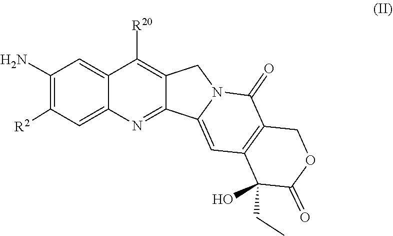

wherein:
   R² is selected from: —H, —CH₃, —CF₃, —F, —Br, —Cl, —OH, —OCH₃ and —OCF₃;
   R²⁰ is selected from: —H, —C₁-C₆ alkyl, —C₃-C₈ cycloalkyl, —(C₁-C₆ alkyl)-O—R⁵,

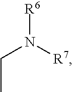

—CO₂R⁸, -aryl, -heteroaryl, —(C₁-C₆ alkyl)-aryl,

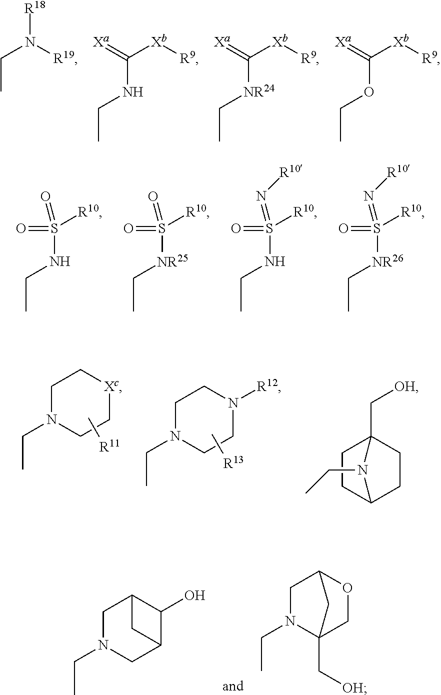

R⁵ is selected from: —H, —C₁-C₆ alkyl, —C₃-C₈ cycloalkyl, -aryl, -heteroaryl and —(C₁-C₆ alkyl)-aryl;
R⁶ and R⁷ are each independently selected from: —H, —C₁-C₆ alkyl, —C₃-C₈ cycloalkyl, —(C₁-C₆ alkyl)-O—R⁵, —C₃-C₈ heterocycloalkyl and —C(O)R⁷;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, —NR¹⁴R^{14′},-aryl, -heteroaryl and —(C₁-C₆ alkyl)-aryl;
R^{10′}is selected from: —H, —C₁-C₆ alkyl, —C₃-C₈ cycloalkyl, -aryl, -heteroaryl, and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl,-S(O)₂R¹⁶ and

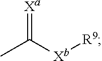

R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁷ is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R¹⁸ and R¹⁹ taken together with the N atom to which they are bonded form a 4-, 5-, 6-, or 7-membered ring having 0 to 3 substituents selected from: halogen, —C₁-C₆ alkyl, —C₃-C₈ cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, O and S, and
X^{c} is selected from: 0, S and S(O)₂,
with the proviso that the compound is other than (S)-9-amino-11-butyl-4-ethyl-4-hydroxy-1,12-dihydro-14H-pyrano[3′,4′:6,7]indolizino[1,2-b]quinoline-3,14(4H)-dione.

In some embodiments, in compounds of Formula (II), R² is selected from: —CH₃, —CF₃,—F, —Br, —Cl, —OH, —OCH₃ and —OCF₃.

In some embodiments, in compounds of Formula (II), R² is selected from: —CH₃, —CF₃, —F, —Cl, —OCH₃ and —OCF₃.

In some embodiments, in compounds of Formula (II), R² is selected from F and Cl.

In some embodiments, in compounds of Formula (II), R²⁰ is selected from: —H, —C₁-C₆ alkyl, —(C₁-C₆ alkyl)-O—R⁵,

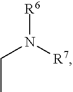

—(C₁-C₆ alkyl)-aryl,

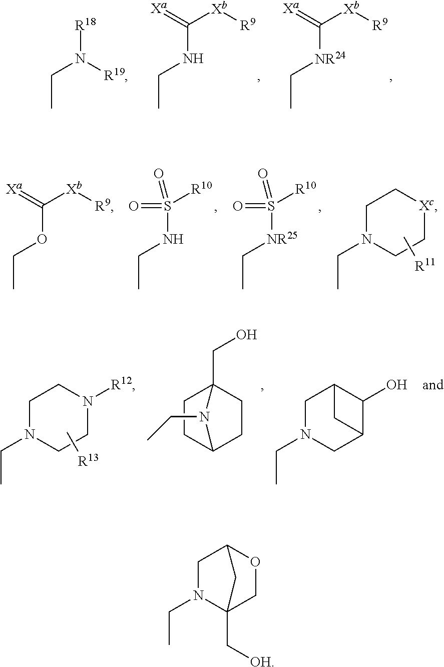

In some embodiments, in compounds of Formula (II), R²⁰ is selected from: —H, —C₁-C₆ alkyl, —(C₁-C₆ alkyl)-O—R⁵,

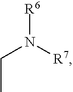

—(C₁-C₆ alkyl)-aryl,

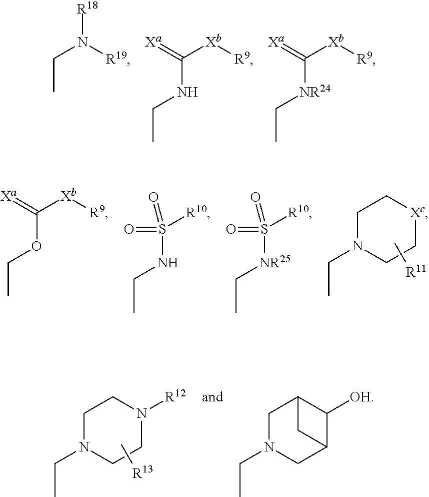

In some embodiments, in compounds of Formula (II), R²⁰ is selected from: —H, —C₁-C₆

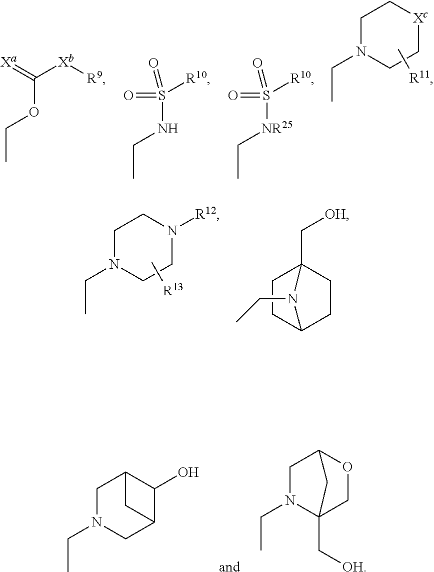

In some embodiments, in compounds of Formula (II), R²⁰ is selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵,

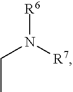

—CO₂R⁸, unsubstituted aryl, -aminoaryl, -heteroaryl, —(C₁-C₆ alkyl)-aminoaryl,

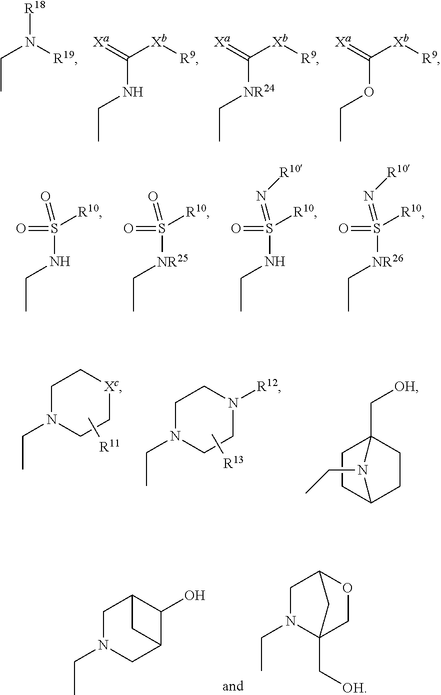

In some embodiments, in compounds of Formula (II), R² is selected from: —CH₃, —CF₃, —F, —Br, —Cl, —OH, —OCH₃ and —OCF₃, and R²⁰ is selected from: —H, —C₁-C₆ alkyl, —(C₁-C₆ alkyl)-O—

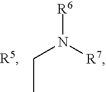

—(C₁-C₆ alkyl)-aryl,

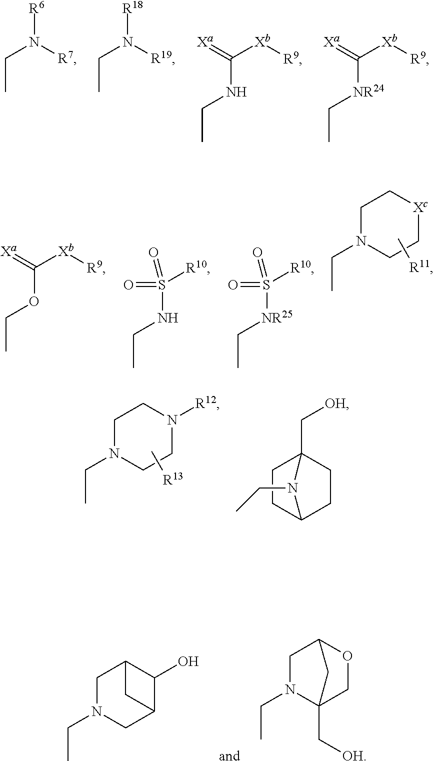

In some embodiments, in compounds of Formula (II), R² is selected from: —CH₃, —CF₃, —F, —Br, —Cl, —OH, —OCH₃ and —OCF₃, and R²⁰ is selected from: —H, —C₁-C₆ alkyl, —(C₁-C₆ alkyl)-O—R⁵,

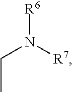

—(C₁-C₆ alkyl)-aryl,

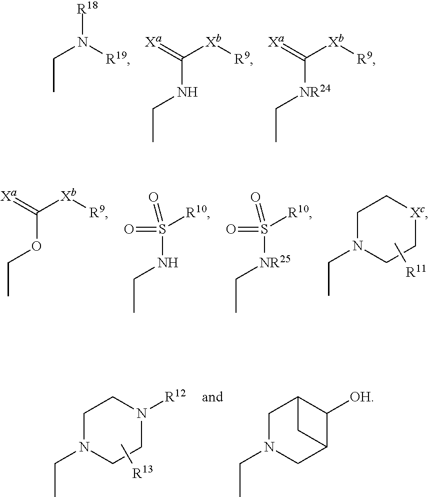

In some embodiments, in compounds of Formula (II), R² is selected from: —CH₃, —CF₃, —F, —Br, —Cl, —OH, —OCH₃ and —OCF₃, and R²⁰ is selected from: —H, —C₁-C₆ alkyl, —(C₁-C₆ alkyl)-O—

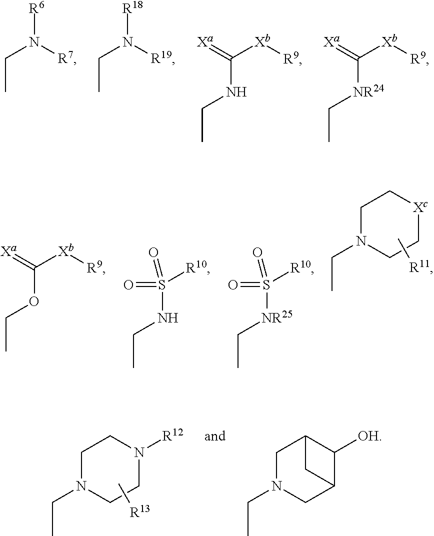

In some embodiments, in compounds of Formula (II), R⁵ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted-aryl, -aminoaryl, -heteroaryl and —(C₁-C₆ alkyl)-aminoaryl.

In some embodiments, in compounds of Formula (II), R⁶ and R⁷ are each independently selected from: —H, —C₁-C₆ alkyl, —C₃-C₈ cycloalkyl and —C(O)R¹⁷.

In some embodiments, in compounds of Formula (II), R⁶ is H, and R⁷ is selected from: —H,-C₁-C₆ alkyl, —C₃-C₈ cycloalkyl, —(C₁-C₆ alkyl)-O—R⁵, —C₃-C₈ heterocycloalkyl and —C(O)R¹⁷.

In some embodiments, in compounds of Formula (II), R⁶ is H, and R⁷ is selected from: —H, —C₁-C₆ alkyl, —C₃-C₈ cycloalkyl and —C(O)R¹⁷.

In some embodiments, in compounds of Formula (II), R⁶ and R⁷ are each independently selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵, —C₃-C₈ heterocycloalkyl and —C(O)R¹⁷.

In some embodiments, in compounds of Formula (II), R⁸ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (II), each R⁹ is independently selected from: —C₁-C₆ alkyl, —C₃-C₈ cycloalkyl, -aryl and —(C₁-C₆ alkyl)-aryl.

In some embodiments, in compounds of Formula (II), each R⁹ is independently selected from: —C₁-C₆ alkyl and —(C₁-C₆ alkyl)-aryl.

In some embodiments, in compounds of Formula (II), each R⁹ is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted-aryl, -aminoaryl, -heteroaryl and —(C₁-C₆ alkyl)-aminoaryl.

In some embodiments, in compounds of Formula (II), each R¹⁰ is independently selected from: —C₁-C₆ alkyl, —C₃-C₈ cycloalkyl, —NR¹⁴R^{14′}, -aryl and —(C₁-C₆ alkyl)-aryl.

In some embodiments, in compounds of Formula (II), each R¹⁰ is independently selected from: —C₁-C₆ alkyl, —NR¹⁴R^{14′}, -aryl and —(C₁-C₆ alkyl)-aryl.

In some embodiments, in compounds of Formula (II), each R¹⁰ is independently selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, —NR¹⁴R^{14′}, unsubstituted-aryl, -aminoaryl, -heteroaryl and —(C₁-C₆ alkyl)-aryl.

In some embodiments, in compounds of Formula (II), R^{10′} is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted-aryl, -aminoaryl, -heteroaryl and —(C₁-C₆ alkyl)-aryl.

In some embodiments, in compounds of Formula (II), R¹¹ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (II), R¹² is selected from: —H, —C₁-C₆ alkyl, —CO₂R⁸, -aryl, —(C₁-C₆ alkyl)-aryl and —S(O)₂R¹⁶.

In some embodiments, in compounds of Formula (II), R¹² is selected from: —H, unsubstituted —C₁-C₆ alkyl, —C₁-C₆ haloalkyl, —C₁-C₆ hydroxyalkyl, —C₁-C₆ aminoalkyl, —CO₂R⁸, unsubstituted-aryl, -aminoaryl, -heteroaryl, —(C₁-C₆ alkyl)-aminoaryl, —S(O)₂R¹⁶ and

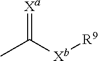

In some embodiments, in compounds of Formula (II), R¹³ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (II), R¹⁴ and R^{14′} are each independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (II), R¹⁶ is selected from: -aryl, -heteroaryl and —(C₁-C₆ alkyl)-aryl.

In some embodiments, in compounds of Formula (II), R¹⁶ is selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted-aryl, -aminoaryl, -heteroaryl and —(C₁-C₆ alkyl)-aminoaryl.

In some embodiments, in compounds of Formula (II), R¹⁷ is —C₁-C₆ alkyl.

In some embodiments, in compounds of Formula (II), R¹⁷ is selected from: unsubstituted C₁-C₆ alkyl, —C₁-C₆ hydroxyalkyl, —C₃-C₈ cycloalkyl, —C₃-C₈ heterocycloalkyl, —(C₁-C₆ alkyl)-C₃-C₈ heterocycloalkyl, unsubstituted aryl, -hydroxyaryl, -aminoaryl, -heteroaryl and —(C₁-C₆ alkyl)-aminoaryl.

In some embodiments, in compounds of Formula (II), R¹⁸ and R¹⁹ taken together with the N atom to which they are bonded form a 4-, 5-, 6- or 7-membered ring having 0 to 3 substituents selected from: halogen, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —(C₁-C₆ alkyl)-O—R⁵.

In some embodiments, in compounds of Formula (II), X^{a} and X^{b} are each independently selected from: NH and O.

Combinations of any of the foregoing embodiments for compounds of Formula (II) are also contemplated and each combination forms a separate embodiment for the purposes of the present disclosure.

In certain embodiments, the compound of Formula (I) has Formula (III):

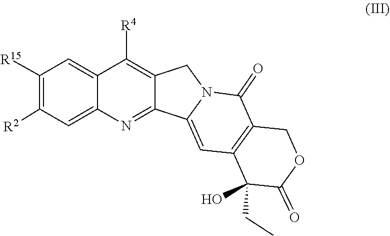

wherein:
   R² is selected from: —H, —CH₃, —CF₃, —F, —Br, —Cl, —OH, —OCH₃ and —OCF₃;
   R¹⁵ is selected from: —H, —CH₃, —CHF₂, —CF₃, —F, —Br, —Cl, —OH, —OCH₃ and —OCF₃;
   R⁴ is selected from:

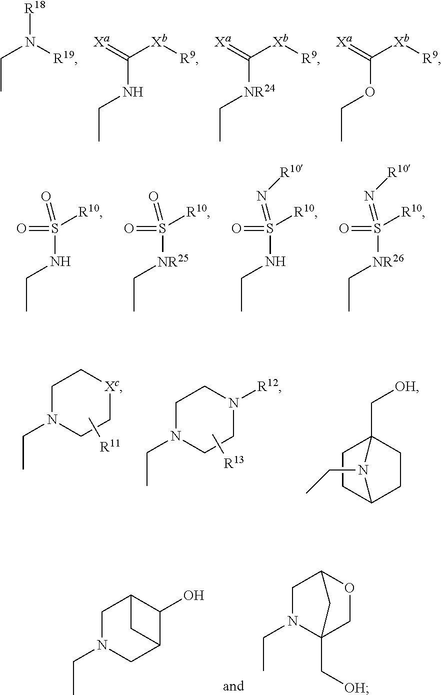

R⁵ is selected from: —H, —C₁-C₆ alkyl, —C₃-C₈ cycloalkyl, -aryl, -heteroaryl and —(C₁-C₆ alkyl)-aryl;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, —NR¹⁴R^{14′},-aryl, -heteroaryl and —(C₁-C₆ alkyl)-aryl;
R^{10′} is selected from: —H, —C₁-C₆ alkyl, —C₃-C₈ cycloalkyl, -aryl, -heteroaryl and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl,-S(O)₂R¹⁶ and

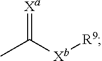

R¹³ is selected from: —H and —C₁-C₆ alkyl;
   R¹⁴ and R^{14′} are each independently selected from: —H, C₁-C₆ alkyl, —C₃-C₈ cycloalkyl and —C₃-C₈ heterocycloalkyl;
   R¹⁶ is selected from: —C₁-C₆ alkyl, —C₃-C₈ cycloalkyl, -aryl, -heteroaryl and —(C₁-C₆ alkyl)-aryl;
   R¹⁸ and R¹⁹ taken together with the N atom to which they are bonded form a 4-, 5-, 6-, or 7-membered ring having 0 to 3 substituents selected from: halogen, —C₁-C₆ alkyl, —C₃-C₈ cycloalkyl and —(C₁-C₆ alkyl)-O—R⁵;
   R²⁴, R²⁵ and R²⁶ are each —C₁-C₆ alkyl;
   X^{a} and X^{b} are each independently selected from: NH, 0 and S, and
   X^{c} is selected from: 0, S and S(O)₂.

In some embodiments, in compounds of Formula (III), R² is selected from: —H, —CH₃, —CF₃, —F, —Cl, —OCH₃ and —OCF₃.

In some embodiments, in compounds of Formula (III), R² is selected from: —H, —F and —Cl.

In some embodiments, in compounds of Formula (III), R¹⁵ is selected from: —CH₃, —CF₃,-OCH₃ and —OCF₃.

In some embodiments, in compounds of Formula (III), R¹⁵ is selected from: —CH₃ and —OCH₃.

In some embodiments, in compounds of Formula (III), R² is selected from: —H, —F and —Cl, and R¹⁵ is selected from: —CH₃, —CF₃, —OCH₃ and —OCF₃.

In some embodiments, in compounds of Formula (III), R² is selected from: —H, —F and —Cl, and R¹⁵ is selected from: —CH₃ and —OCH₃.

In some embodiments, in compounds of Formula (III), R⁴ is selected from:

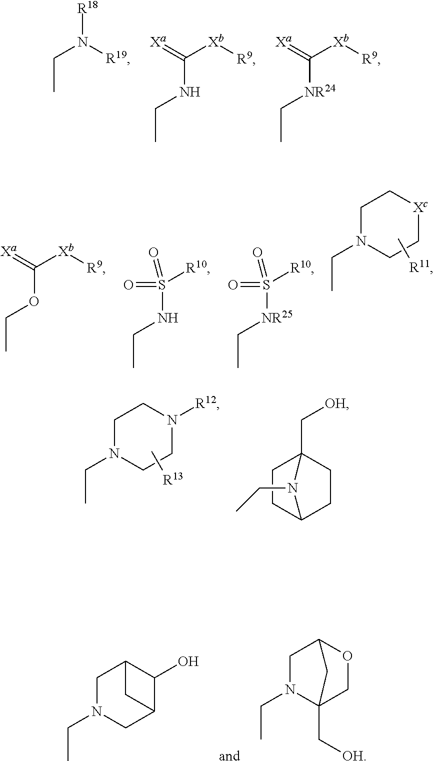

In some embodiments, in compounds of Formula (III), R⁵ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted-aryl, -aminoaryl, -heteroaryl and —(C₁-C₆ alkyl)-aminoaryl.

In some embodiments, in compounds of Formula (III), R⁸ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (III), each R⁹ is independently selected from: —C₁-C₆ alkyl, —C₃-C₈ cycloalkyl, -aryl and —(C₁-C₆ alkyl)-aryl.

In some embodiments, in compounds of Formula (III), each R⁹ is independently selected from: —C₁-C₆ alkyl and —(C₁-C₆ alkyl)-aryl.

In some embodiments, in compounds of Formula (III), each R⁹ is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted-aryl, -aminoaryl, -heteroaryl and —(C₁-C₆ alkyl)-aminoaryl.

In some embodiments, in compounds of Formula (III), each R¹⁰ is independently selected from: —C₁-C₆ alkyl, —C₃-C₈ cycloalkyl, —NR¹⁴R^{14′}, -aryl and —(C₁-C₆ alkyl)-aryl.

In some embodiments, in compounds of Formula (III), each R¹⁰ is independently selected from: —C₁-C₆ alkyl, —NR¹⁴R^{14′}, -aryl and —(C₁-C₆ alkyl)-aryl.

In some embodiments, in compounds of Formula (III), each R¹⁰ is independently selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, —NR¹⁴R^{14′}, unsubstituted-aryl, -aminoaryl, -heteroaryl and —(C₁-C₆ alkyl)-aryl.

In some embodiments, in compounds of Formula (III), R^{10′} is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted-aryl, -aminoaryl, -heteroaryl and —(C₁-C₆ alkyl)-aryl.

In some embodiments, in compounds of Formula (III), R¹¹ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (III), R¹² is selected from: —H, —C₁-C₆ alkyl, —CO₂R⁸, -aryl, —(C₁-C₆ alkyl)-aryl and —S(O)₂R¹⁶.

In some embodiments, in compounds of Formula (III), R¹² is selected from: —H, unsubstituted —C₁-C₆ alkyl, —C₁-C₆ haloalkyl, —C₁-C₆ hydroxyalkyl, —C₁-C₆ aminoalkyl, —CO₂R⁸, unsubstituted-aryl,-aminoaryl, -heteroaryl, —(C₁-C₆ alkyl)-aminoaryl,—S(O)₂R¹⁶ and

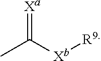

In some embodiments, in compounds of Formula (III), R¹³ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (III), R¹⁴ and R^{14′} are each independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (III), R¹⁶ is selected from: -aryl, -heteroaryl and —(C₁-C₆ alkyl)-aryl.

In some embodiments, in compounds of Formula (III), R¹⁶ is selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted-aryl, -aminoaryl, -heteroaryl and —(C₁-C₆ alkyl)-aminoaryl.

In some embodiments, in compounds of Formula (III), R¹⁸ and R¹⁹ taken together with the N atom to which they are bonded form a 4-, 5-, 6- or 7-membered ring having 0 to 3 substituents selected from: halogen, unsubstituted C₁-C₆ alkyl, —C₁-C₆ haloalkyl, —C₁-C₆ hydroxyalkyl, —C₁-C₆ aminoalkyl, —C₃-C₈ cycloalkyl and —(C₁-C₆ alkyl)-O—R⁵.

In some embodiments, in compounds of Formula (III), X^{a} and X^{b} are each independently selected from: NH and O.

Combinations of any of the foregoing embodiments for compounds of Formula (III) are also contemplated and each combination forms a separate embodiment for the purposes of the present disclosure.

In certain embodiments, each alkyl, cycloalkyl, heterocycloalkyl, aryl and heteroaryl group as defined in any one of Formulae (I), (II) or (III) is optionally substituted with one or more substituents selected from: halogen, acyl, acyloxy, alkoxy, carboxy, hydroxy, amino, amido, nitro, cyano, azido, alkylthio, thio, sulfonyl, sulfonamido, alkyl, cycloalkyl, heterocycloalkyl, aryl and heteroaryl. In some embodiments, each alkyl, cycloalkyl, heterocycloalkyl, aryl and heteroaryl group as defined in any one of Formulae (I), (II) or (III) is optionally substituted with one or more substituents selected from: halogen, acyl, acyloxy, alkoxy, carboxy, hydroxy, amino, amido, nitro, cyano, azido, alkylthio, thio, sulfonyl and sulfonamido.

In certain embodiments, the camptothecin analogue comprised by the ADC according to the present disclosure is a compound having Formula (I) and is selected from the compounds shown in Tables 5 and 6.

In certain embodiments, the camptothecin analogue is a compound having Formula (II).

In some embodiments, the camptothecin analogue is a compound having Formula (II), in which R² is F, and R²⁰ is H, —(C₁-C₆)—O—R⁵ or

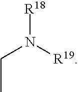

In some embodiments, the camptothecin analogue is a compound having Formula (II), in which R² is F; R²⁰ is H, —(C₁-C₆)—O—R^{S} or

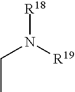

R⁵ is H, and R¹⁸ and R¹⁹ taken together with the N atom to which they are bonded form an unsubstituted 4-, 5-, 6-, or 7-membered ring. In some embodiments, the camptothecin analogue is a compound having Formula (II), in which R² is F; R²⁰ is —(C₁-C₆)—O—R⁵, and R⁵ is H. In certain embodiments, the camptothecin analogue is a compound having Formula (II) and is selected from the compounds shown in Table 5.

In certain embodiments, the camptothecin analogue is a compound having Formula (III).

In certain embodiments, the camptothecin analogue is a compound having Formula (III), in which R² is F; R¹⁵ is —CH₃; R⁴ is

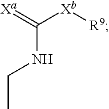

R⁹ is —C₁-C₆ hydroxyalkyl, and X^{a} and X^{b} are each O. In certain embodiments, the camptothecin analogue is a compound having Formula (III) and is selected from the compounds shown in Table 6.

In certain embodiments, the camptothecin analogue comprised by the ADC according to the present disclosure is Compound 139, Compound 140, Compound 141 or Compound 148. In some embodiments, the camptothecin analogue comprised by the ADC according to the present disclosure is Compound 139 or Compound 141.

TABLE 5
Exemplary Camptothecin Analogues of Formula (II)

| | Compound | |
|---|---|---|
| Structure | Name | Number |
| 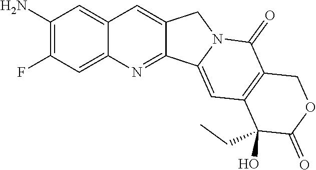 [SMILES: CC[C@@]1(O)C(=O)OCc2c1cc1n(c2=O)Cc2cc3cc(N)c(F)cc3nc2-1] | (S)-9-amino-4-ethyl-8-fluoro-4-hydroxy- 1,12-dihydro-14H- pyrano[3′,4′:6,7]indolizino[1,2- b]quinoline-3,14(4H)-dione | Compound 140 |
| 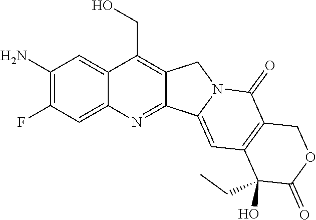 [SMILES: CC[C@@]1(O)C(=O)OCc2c1cc1n(c2=O)Cc2c-1nc1cc(F)c(N)cc1c2CO] | (S)-9-amino-4-ethyl-8-fluoro-4-hydroxy- 11-(hydroxymethyl)-1,12-dihydro-14H- pyrano[3′,4′:6,7]indolizino[1,2- b]quinoline-3,14(4H)-dione | Compound 141 |
| 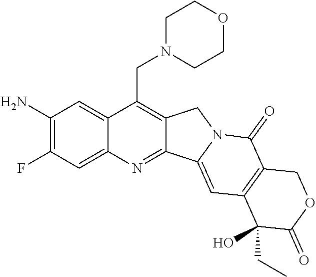 [SMILES: CC[C@@]1(O)C(=O)OCc2c1cc1n(c2=O)Cc2c-1nc1cc(F)c(N)cc1c2CN1CCOCC1] | (S)-9-amino-4-ethyl-8-fluoro-4-hydroxy- 11-(morpholinomethyl)-1,12-dihydro-14H- pyrano[3′,4′:6,7]indolizino[1,2- b]quinoline-3,14(4H)-dione | Compound 142 |
| 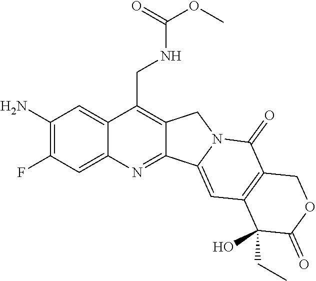 [SMILES: CC[C@@]1(O)C(=O)OCc2c1cc1n(c2=O)Cc2c-1nc1cc(F)c(N)cc1c2CNC(=O)OC] | methyl (S)-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)carbamate | Compound 143 |
| 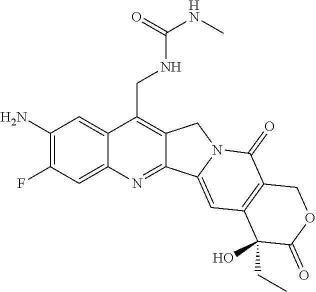 [SMILES: CC[C@@]1(O)C(=O)OCc2c1cc1n(c2=O)Cc2c-1nc1cc(F)c(N)cc1c2CNC(=O)NC] | (S)-1-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-3-methylurea | Compound 144 |
| 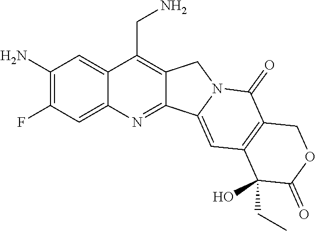 [SMILES: CC[C@@]1(O)C(=O)OCc2c1cc1n(c2=O)Cc2c-1nc1cc(F)c(N)cc1c2CN] | (S)-9-amino-11-(aminomethyl)-4-ethyl-8- fluoro-4-hydroxy-1,12-dihydro-14H- pyrano[3′,4′:6,7]indolizino[1,2- b]quinoline-3,14(4H)-dione | Compound 145 |
| 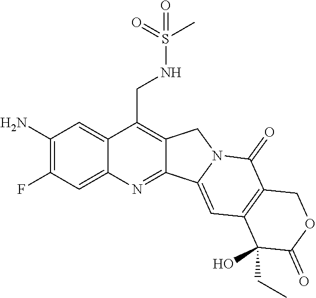 [SMILES: CC[C@@]1(O)C(=O)OCc2c1cc1n(c2=O)Cc2c-1nc1cc(F)c(N)cc1c2CNS(C)(=O)=O] | (S)-N-((9-amino-4-ethyl-8-fluoro-4- hydroxy-3,14-dioxo-3,4,12,14-tetrahydro- 1H-pyrano[3′,4′:6,7]indolizino[1,2- b]quinolin-11- yl)methyl)methanesulfonamide | Compound 146 |
| 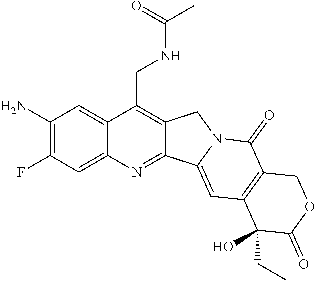 [SMILES: CC[C@@]1(O)C(=O)OCc2c1cc1n(c2=O)Cc2c-1nc1cc(F)c(N)cc1c2CNC(C)=O] | (S)-N-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)acetamide | Compound 147 |
| 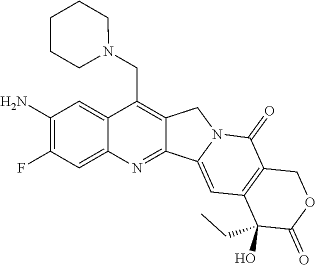 [SMILES: CC[C@@]1(O)C(=O)OCc2c1cc1n(c2=O)Cc2c-1nc1cc(F)c(N)cc1c2CN1CCCCC1] | (S)-9-amino-4-ethyl-8-fluoro-4-hydroxy- 11-(piperidin-1-ylmethyl)-1,12-dihydro- 14H-pyrano[3′,4′:6,7]indolizino[1,2- b]quinoline-3,14(4H)-dione | Compound 148 |
| 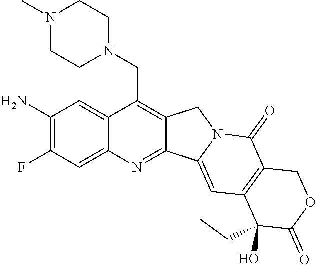 [SMILES: CC[C@@]1(O)C(=O)OCc2c1cc1n(c2=O)Cc2c-1nc1cc(F)c(N)cc1c2CN1CCN(C)CC1] | (S)-9-amino-4-ethyl-8-fluoro-4-hydroxy- 11-((4-methylpiperazin-1-yl)methyl)-1,12- dihydro-14H- pyrano[3′,4′:6,7]indolizino[1,2- b]quinoline-3,14(4H)-dione | Compound 149 |
| 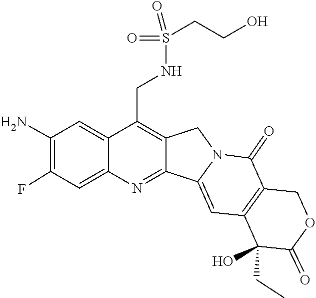 [SMILES: CC[C@@]1(O)C(=O)OCc2c1cc1n(c2=O)Cc2c-1nc1cc(F)c(N)cc1c2CNS(=O)(=O)CCO] | (S)-N-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-2- hydroxyethane-1-sulfonamide | Compound 150 |
| 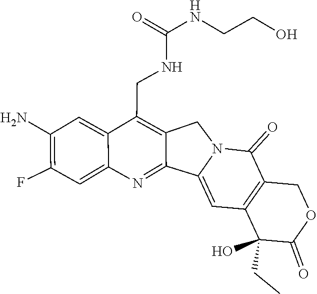 [SMILES: CC[C@@]1(O)C(=O)OCc2c1cc1n(c2=O)Cc2c-1nc1cc(F)c(N)cc1c2CNC(=O)NCCO] | (S)-1-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-3-(2- hydroxyethyl)urea | Compound 151 |
| 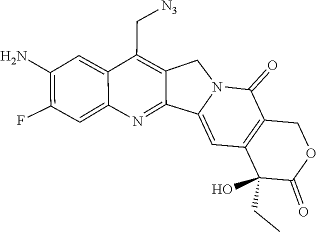 [SMILES: CC[C@@]1(O)C(=O)OCc2c1cc1n(c2=O)Cc2c-1nc1cc(F)c(N)cc1c2CN=[N+]=[N-]] | (S)-9-amino-11-(azidomethyl)-4-ethyl-8- fluoro-4-hydroxy-1,12-dihydro-14H- pyrano[3′,4′:6,7]indolizino[1,2- b]quinoline-3,14(4H)-dione | Compound 152 |
| 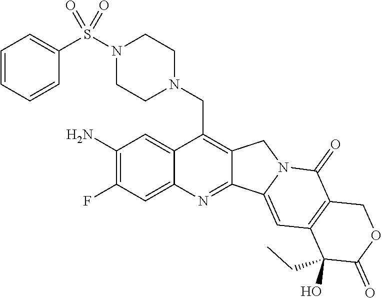 [SMILES: CC[C@@]1(O)C(=O)OCc2c1cc1n(c2=O)Cc2c-1nc1cc(F)c(N)cc1c2CN1CCN(S(=O)(=O)c2ccccc2)CC1] | (S)-9-amino-4-ethyl-8-fluoro-4-hydroxy- 11-((4-(phenylsulfonyl)piperazin-1- yl)methyl)-1,12-dihydro-14H- pyrano[3′,4′:6,7]indolizino[1,2- b]quinoline-3,14(4H)-dion | Compound 153 |
| 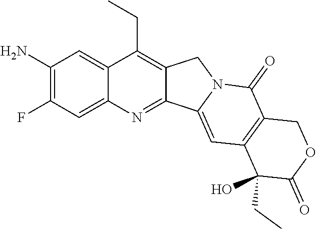 [SMILES: CCc1c2c(nc3cc(F)c(N)cc13)-c1cc3c(c(=O)n1C2)COC(=O)[C@]3(O)CC] | (S)-9-amino-4,11-diethyl-8-fluoro-4- hydroxy-1,12-dihydro-14H- pyrano[3′,4′:6,7]indolizino[1,2- b]quinoline-3,14(4H)-dione | Compound 154 |
| 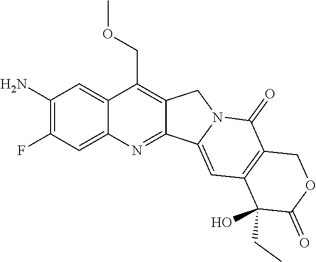 [SMILES: CC[C@@]1(O)C(=O)OCc2c1cc1n(c2=O)Cc2c-1nc1cc(F)c(N)cc1c2COC] | (S)-9-amino-4-ethyl-8-fluoro-4-hydroxy- 11-(methoxymethyl)-1,12-dihydro-14H- pyrano[3′,4′:6,7]indolizino[1,2- b]quinoline-3,14(4H)-dione | Compound 155 |
| 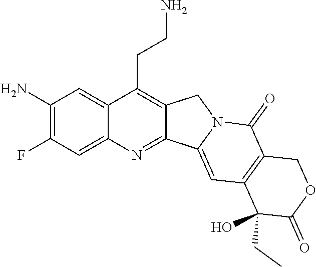 [SMILES: CC[C@@]1(O)C(=O)OCc2c1cc1n(c2=O)Cc2c-1nc1cc(F)c(N)cc1c2CCN] | (S)-9-amino-11-(2-aminoethyl)-4-ethyl-8- fluoro-4-hydroxy-1,12-dihydro-14H- pyrano[3′,4′:6,7]indolizino[1,2- b]quinoline-3,14(4H)-dione | Compound 156 |
| 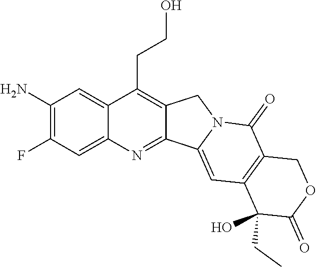 [SMILES: CC[C@@]1(O)C(=O)OCc2c1cc1n(c2=O)Cc2c-1nc1cc(F)c(N)cc1c2CCO] | (S)-9-amino-4-ethyl-8-fluoro-4-hydroxy- 11-(2-hydroxyethyl)-1,12-dihydro-14H- pyrano[3′,4′:6,7]indolizino[1,2- b]quinoline-3,14(4H)-dione | Compound 157 |
| 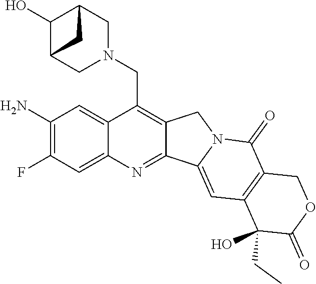 [SMILES: CC[C@@]1(O)C(=O)OCc2c1cc1n(c2=O)Cc2c-1nc1cc(F)c(N)cc1c2CN1C[C@H]2C[C@@H](C1)C2O] | (4S)-9-amino-4-ethyl-8-fluoro-4-hydroxy- 11-(((1R,5S)-6-hydroxy-3- azabicyclo[3.1.1]heptan-3-yl)methyl)-1,12- dihydro-14HI- pyrano[3′,4′:6,7]indolizino[1,2- b]quinoline-3,14(4H)-dione | Compound 158 |
| 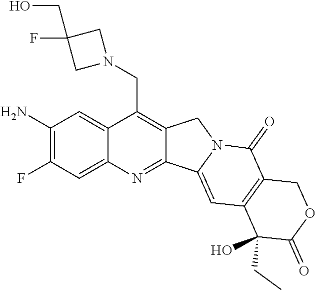 [SMILES: CC[C@@]1(O)C(=O)OCc2c1cc1n(c2=O)Cc2c-1nc1cc(F)c(N)cc1c2CN1CC(F)(CO)C1] | (S)-9-amino-4-ethyl-8-fluoro-11-((3- fluoro-3-(hydroxymethyl)azetidin-1- yl)methyl)-4-hydroxy-1,12-dihydro-14H- pyrano[3′,4′:6,7]indolizino[1,2- b]quinoline-3,14(4H)-dione | Compound 159 |
| 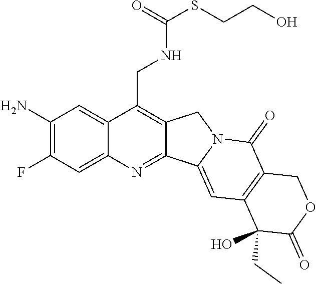 [SMILES: CC[C@@]1(O)C(=O)OCc2c1cc1n(c2=O)Cc2c-1nc1cc(F)c(N)cc1c2CNC(=O)SCCO] | S-(2-hydroxyethyl) (S)-((9-amino-4-ethyl- 8-fluoro-4-hydroxy-3,14-dioxo-3,4,12,14- tetrahydro-1H- pyrano[3′,4′:6,7]indolizino[1,2-b]quinolin- 11-yl)methyl)carbamothioate | Compound 160 |
| 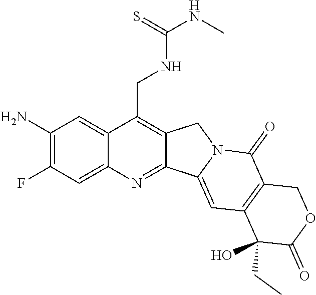 [SMILES: CC[C@@]1(O)C(=O)OCc2c1cc1n(c2=O)Cc2c-1nc1cc(F)c(N)cc1c2CNC(=S)NC] | (S)-1-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-3-methylthiourea | Compound 161 |
| 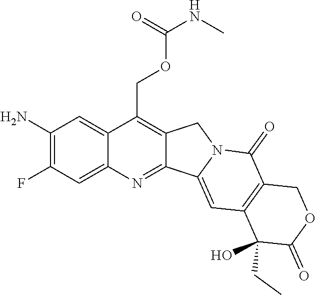 [SMILES: CC[C@@]1(O)C(=O)OCc2c1cc1n(c2=O)Cc2c-1nc1cc(F)c(N)cc1c2COC(=O)NC] | (S)-(9-amino-4-ethyl-8-fluoro-4-hydroxy- 3,14-dioxo-3,4,12,14-tetrahydro-1H- pyrano[3′,4′:6,7]indolizino[1,2-b]quinolin- 11-yl)methyl methylcarbamate | Compound 162 |
| 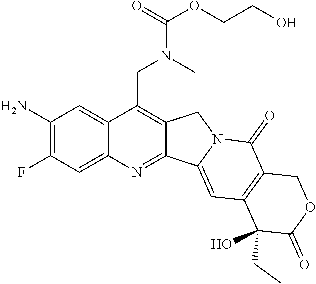 [SMILES: CC[C@@]1(O)C(=O)OCc2c1cc1n(c2=O)Cc2c-1nc1cc(F)c(N)cc1c2CN(C)C(=O)OCCO] | 2-hydroxyethyl (S)-((9-amino-4-ethyl-8- fluoro-4-hydroxy-3,14-dioxo-3,4,12,14- tetrahydro-1H- pyrano[3′,4′:6,7]indolizino[1,2-b]quinolin- 11-yl)methyl)(methyl)carbamate | Compound 163 |
| 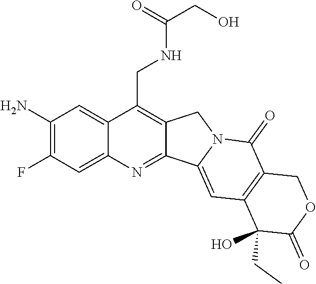 [SMILES: CC[C@@]1(O)C(=O)OCc2c1cc1n(c2=O)Cc2c-1nc1cc(F)c(N)cc1c2CNC(=O)CO] | (S)-N-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-2- hydroxyacetamide | Compound 164 |
| 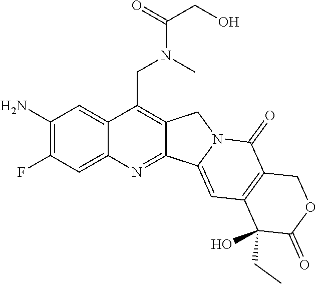 [SMILES: CC[C@@]1(O)C(=O)OCc2c1cc1n(c2=O)Cc2c-1nc1cc(F)c(N)cc1c2CN(C)C(=O)CO] | (S)-N-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-2-hydroxy-N- methylacetamide | Compound 165 |
| 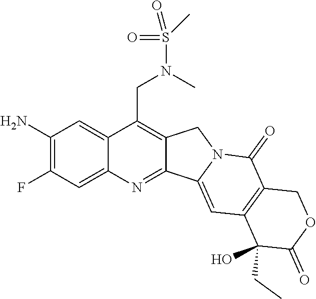 [SMILES: CC[C@@]1(O)C(=O)OCc2c1cc1n(c2=O)Cc2c-1nc1cc(F)c(N)cc1c2CN(C)S(C)(=O)=O] | (S)-N-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-N- methylmethanesulfonamide | Compound 166 |
| 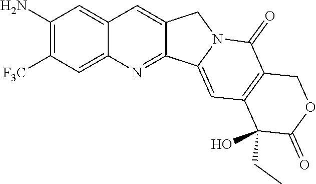 [SMILES: CC[C@@]1(O)C(=O)OCc2c1cc1n(c2=O)Cc2cc3cc(N)c(C(F)(F)F)cc3nc2-1] | (S)-9-amino-4-ethyl-4-hydroxy-8- (trifluoromethyl)-1,12-dihydro-14H- pyrano[3′,4′:6,7]indolizino[1,2- b]quinoline-3,14(4H)-dione | Compound 167 |
| 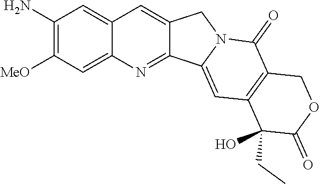 [SMILES: CC[C@@]1(O)C(=O)OCc2c1cc1n(c2=O)Cc2cc3cc(N)c(OC)cc3nc2-1] | (S)-9-amino-4-ethyl-4-hydroxy-8- methoxy-1,12-dihydro-14H- pyrano[3′,4′:6,7]indolizino[1,2- b]quinoline-3,14(4H)-dione | Compound 168 |
| 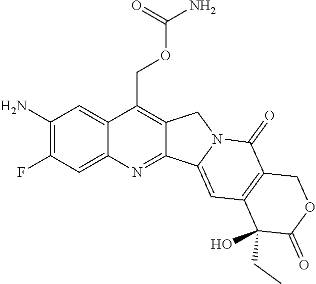 [SMILES: CC[C@@]1(O)C(=O)OCc2c1cc1n(c2=O)Cc2c-1nc1cc(F)c(N)cc1c2COC(N)=O] | (S)-(9-amino-4-ethyl-8-fluoro-4-hydroxy- 3,14-dioxo-3,4,12,14-tetrahydro-1H- pyrano[3′,4′:6,7]indolizino[1,2-b]quinolin- 11-yl)methyl carbamate | Compound 169 |
| 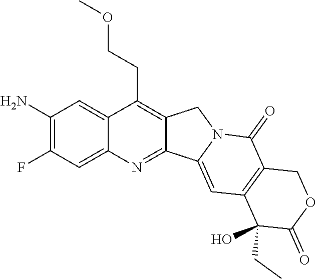 [SMILES: CC[C@@]1(O)C(=O)OCc2c1cc1n(c2=O)Cc2c-1nc1cc(F)c(N)cc1c2CCOC] | (S)-9-amino-4-ethyl-8-fluoro-4-hydroxy- 11-(2-methoxyethyl)-1,12-dihydro-14H- pyrano[3′,4′:6,7]indolizino[1,2- b]quinoline-3,14(4H)-dione | Compound 170 |
| 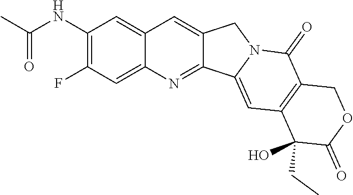 [SMILES: CC[C@@]1(O)C(=O)OCc2c1cc1n(c2=O)Cc2cc3cc(NC(C)=O)c(F)cc3nc2-1] | (S)-N-(4-ethyl-8-fluoro-4-hydroxy-3,14- dioxo-3,4, 12,14-tetrahydro-1H- pyrano[3′,4′:6,7]indolizino[1,2-b]quinolin- 9-yl)acetamide | Compound 171 |

TABLE 6
Exemplary Camptothecin Analogues of Formula (III)

| | Compound | |
|---|---|---|
| Structure | Name | Number |
| 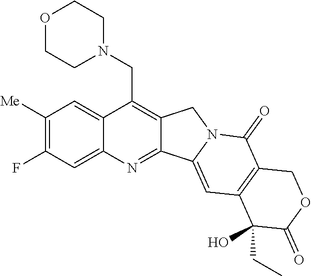 [SMILES: CC[C@@]1(O)C(=O)OCc2c1cc1n(c2=O)Cc2c-1nc1cc(F)c(C)cc1c2CN1CCOCC1] | (S)-4-ethyl-8-fluoro-4-hydroxy-9-methyl- 11-(morpholinomethyl)-1,12-dihydro-14H- pyrano[3′,4′:6,7]indolizino[1,2- b]quinoline-3,14(4H)-dione | Compound 100 |
| 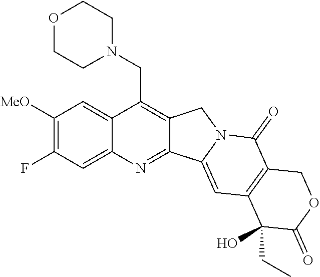 [SMILES: CC[C@@]1(O)C(=O)OCc2c1cc1n(c2=O)Cc2c-1nc1cc(F)c(OC)cc1c2CN1CCOCC1] | (S)-4-ethyl-8-fluoro-4-hydroxy-9-methoxy- 11-(morpholinomethyl)-1,12-dihydro-14H- pyrano[3′,4′:6,7]indolizino[1,2- b]quinoline-3,14(4H)-dione | Compound 101 |
| 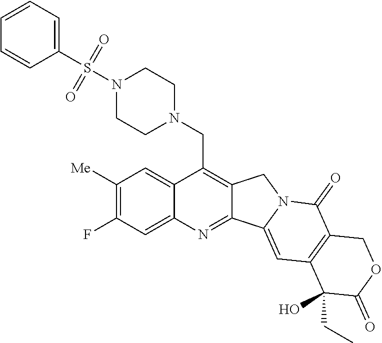 [SMILES: CC[C@@]1(O)C(=O)OCc2c1cc1n(c2=O)Cc2c-1nc1cc(F)c(C)cc1c2CN1CCN(S(=O)(=O)c2ccccc2)CC1] | (S)-4-ethyl-8-fluoro-4-hydroxy-9-methyl- 11-((4-(phenylsulfonyl)piperazin-1- yl)methyl)-1,12-dihydro-14H- pyrano[3′,4′:6,7]indolizino[1,2- b]quinoline-3,14(4H)-dione | Compound 102 |
| 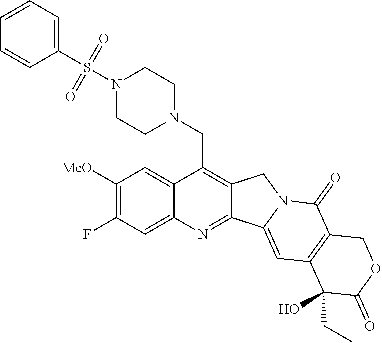 [SMILES: CC[C@@]1(O)C(=O)OCc2c1cc1n(c2=O)Cc2c-1nc1cc(F)c(OC)cc1c2CN1CCN(S(=O)(=O)c2ccccc2)CC1] | (S)-4-ethyl-8-fluoro-4-hydroxy-9-methoxy- 11-((4-(phenylsulfonyl)piperazin-1- yl)methyl)-1,12-dihydro-14H- pyrano[3′,4′:6,7]indolizino[1,2- b]quinoline-3,14(4H)-dione | Compound 103 |
| 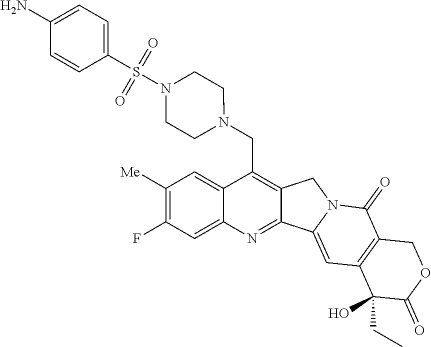 [SMILES: CC[C@@]1(O)C(=O)OCc2c1cc1n(c2=O)Cc2c-1nc1cc(F)c(C)cc1c2CN1CCN(S(=O)(=O)c2ccc(N)cc2)CC1] | (S)-11-((4-((4- aminophenyl)sulfonyl)piperazin-1- yl)methyl)-4-ethyl-8-fluoro-4-hydroxy-9- methyl-1,12-dihydro-14H- pyrano[3′,4′:6,7]indolizino[1,2- b]quinoline-3,14(4H)-dione | Compound 104 |
| 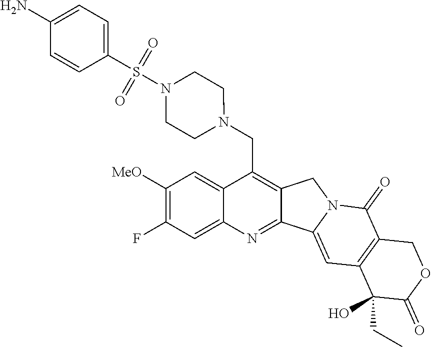 [SMILES: CC[C@@]1(O)C(=O)OCc2c1cc1n(c2=O)Cc2c-1nc1cc(F)c(OC)cc1c2CN1CCN(S(=O)(=O)c2ccc(N)cc2)CC1] | (S)-11-((4-((4- aminophenyl)sulfonyl)piperazin-1- yl)methyl)-4-ethyl-8-fluoro-4-hydroxy-9- methoxy-1,12-dihydro-14H- pyrano[3′,4′:6,7]indolizino[1,2- b]quinoline-3,14(4H)-dione | Compound 105 |
| 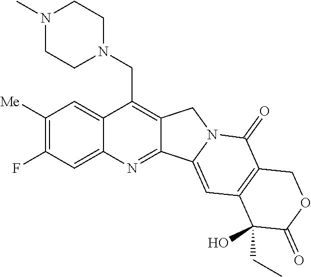 [SMILES: CC[C@@]1(O)C(=O)OCc2c1cc1n(c2=O)Cc2c-1nc1cc(F)c(C)cc1c2CN1CCN(C)CC1] | (S)-4-ethyl-8-fluoro-4-hydroxy-9-methyl- 11-((4-methylpiperazin-1-yl)methyl)-1,12- dihydro-14H- pyrano[3′,4′:6,7]indolizino[1,2- b]quinoline-3,14(4H)-dione | Compound 106 |
| 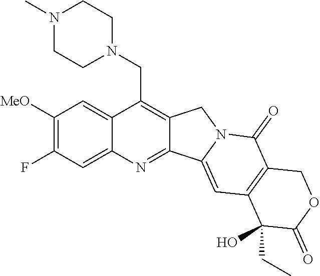 [SMILES: CC[C@@]1(O)C(=O)OCc2c1cc1n(c2=O)Cc2c-1nc1cc(F)c(OC)cc1c2CN1CCN(C)CC1] | (S)-4-ethyl-8-fluoro-4-hydroxy-9-methoxy- 11-((4-methylpiperazin-1-yl)methyl)-1,12- dihydro-14H- pyrano[3′,4′:6,7]indolizino[1,2- b]quinoline-3,14(4H)-dione | Compound 107 |
| 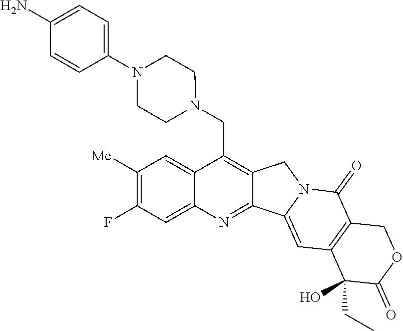 [SMILES: CC[C@@]1(O)C(=O)OCc2c1cc1n(c2=O)Cc2c-1nc1cc(F)c(C)cc1c2CN1CCN(c2ccc(N)cc2)CC1] | (S)-11-((4-(4-aminophenyl)piperazin-1- yl)methyl)-4-ethyl-8-fluoro-4-hydroxy-9- methyl-1,12-dihydro-14H- pyrano[3′,4′:6,7]indolizino[1,2- b]quinoline-3,14(4H)-dione | Compound 108 |
| 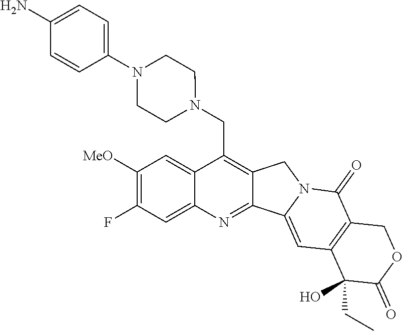 [SMILES: CC[C@@]1(O)C(=O)OCc2c1cc1n(c2=O)Cc2c-1nc1cc(F)c(OC)cc1c2CN1CCN(c2ccc(N)cc2)CC1] | (S)-11-((4-(4-aminophenyl)piperazin-1- yl)methyl)-4-ethyl-8-fluoro-4-hydroxy-9- methoxy-1,12-dihydro-14H- pyrano[3′,4′:6,7]indolizino[1,2- b]quinoline-3,14(4H)-dione | Compound 109 |
| 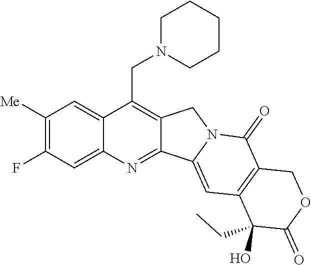 [SMILES: CC[C@@]1(O)C(=O)OCc2c1cc1n(c2=O)Cc2c-1nc1cc(F)c(C)cc1c2CN1CCCCC1] | (S)-4-ethyl-8-fluoro-4-hydroxy-9-methyl- 11-(piperidin-1-ylmethyl)-1,12-dihydro- 14H-pyrano[3′,4′:6,7]indolizino[1,2- b]quinoline-3,14(4H)-dione | Compound 110 |
| 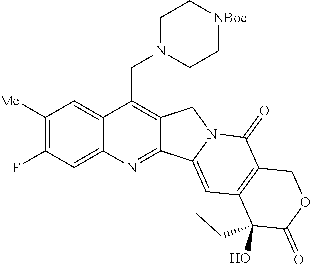 [SMILES: CC[C@@]1(O)C(=O)OCc2c1cc1n(c2=O)Cc2c-1nc1cc(F)c(C)cc1c2CN1CCN(C(=O)OC(C)(C)C)CC1] | tert-butyl (S)-4-((4-ethyl-8-fluoro-4- hydroxy-9-methyl-3,14-dioxo-3,4,12,14- tetrahydro-1H- pyrano[3′,4′:6,7]indolizino[1,2-b]quinolin- 11-yl)methyl)piperazine-1-carboxylate | Compound 111 |
| 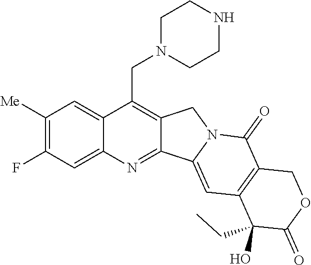 [SMILES: CC[C@@]1(O)C(=O)OCc2c1cc1n(c2=O)Cc2c-1nc1cc(F)c(C)cc1c2CN1CCNCC1] | (S)-4-ethyl-8-fluoro-4-hydroxy-9-methyl- 11-(piperazin-1-ylmethyl)-1,12-dihydro- 14H-pyrano[3′,4′:6,7]indolizino[1,2- b]quinoline-3,14(4H)-dione | Compound 112 |
| 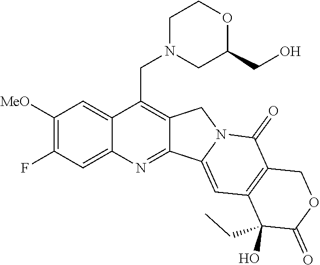 [SMILES: CC[C@@]1(O)C(=O)OCc2c1cc1n(c2=O)Cc2c-1nc1cc(F)c(OC)cc1c2CN1CCO[C@@H](CO)C1] | (S)-4-ethyl-8-fluoro-4-hydroxy-11-(((R)-2- (hydroxymethyl)morpholino)methyl)-9- methyl-1,12-dihydro-14H- pyrano[3′,4′:6,7]indolizino[1,2- b]quinoline-3,14(4H)-dione | Compound 113 |
| 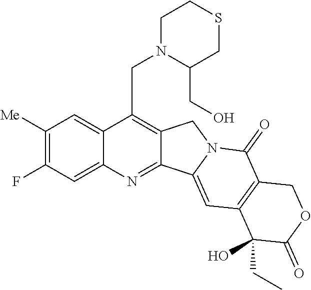 [SMILES: CC[C@@]1(O)C(=O)OCc2c1cc1n(c2=O)Cc2c-1nc1cc(F)c(C)cc1c2CN1CCSCC1CO] | (4S)-4-ethyl-8-fluoro-4-hydroxy-11-((3- (hydroxymethyl)thiomorpholino)methyl)- 9-methyl-1,12-dihydro-14H- pyrano[3′,4′:6,7]indolizino[1,2- b]quinoline-3,14(4H)-dione | Compound 114 |
| 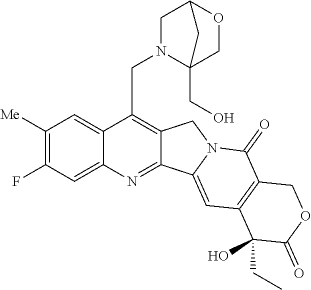 [SMILES: CC[C@@]1(O)C(=O)OCc2c1cc1n(c2=O)Cc2c-1nc1cc(F)c(C)cc1c2CN1CC2CC1(CO)CO2] | (4S)-4-ethyl-8-fluoro-4-hydroxy-11-((4- (hydroxymethyl)-2-oxa-5- azabicyclo[2.2.1]heptan-5-yl)methyl)-9- methyl-1,12-dihydro-14H- pyrano[3′,4′:6,7]indolizino[1,2- b]quinoline-3,14(4H)-dione | Compound 115 |
| 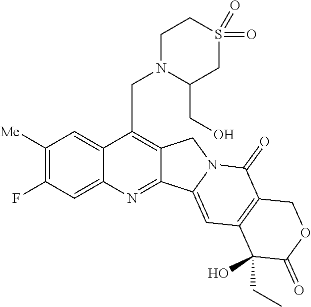 [SMILES: CC[C@@]1(O)C(=O)OCc2c1cc1n(c2=O)Cc2c-1nc1cc(F)c(C)cc1c2CN1CCS(=O)(=O)CC1CO] | (4S)-4-ethyl-8-fluoro-4-hydroxy-11-((3- (hydroxymethyl)-1,1- dioxidothiomorpholino)methyl)-9-methyl- 1,12-dihydro-14H- pyrano[3′,4′:6,7]indolizino[1,2- b]quinoline-3,14(4H)-dione | Compound 116 |
| 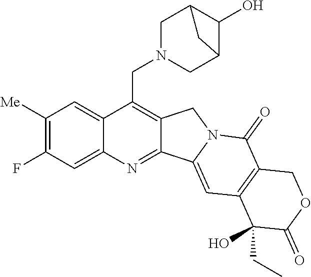 [SMILES: CC[C@@]1(O)C(=O)OCc2c1cc1n(c2=O)Cc2c-1nc1cc(F)c(C)cc1c2CN1CC2CC(C1)C2O] | (4S)-4-ethyl-8-fluoro-4-hydroxy-11-((6- hydroxy-3-azabicyclo[3.1.1]heptan-3- yl)methyl)-9-methyl-1,12-dihydro-14H- pyrano[3′,4′:6,7]indolizino[1,2- b]quinoline-3,14(4H)-dione | Compound 117 |
| 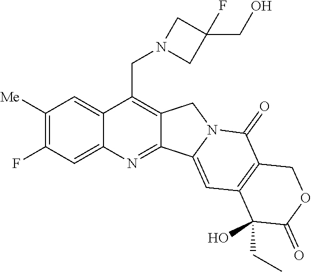 [SMILES: CC[C@@]1(O)C(=O)OCc2c1cc1n(c2=O)Cc2c-1nc1cc(F)c(C)cc1c2CN1CC(F)(CO)C1] | (S)-4-ethyl-8-fluoro-11-((3-fluoro-3- (hydroxymethyl)azetidin-1-yl)methyl)-4- hydroxy-9-methyl-1,12-dihydro-14H- pyrano[3′,4′:6,7]indolizino[1,2- b]quinoline-3,14(4H)-dione | Compound 118 |
| 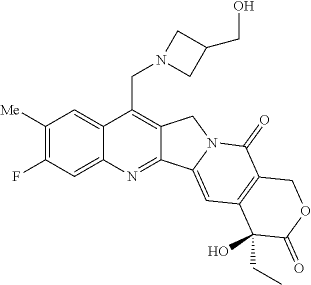 [SMILES: CC[C@@]1(O)C(=O)OCc2c1cc1n(c2=O)Cc2c-1nc1cc(F)c(C)cc1c2CN1CC(CO)C1] | (S)-4-ethyl-8-fluoro-4-hydroxy-11-((3- (hydroxymethyl)azetidin-1-yl)methyl)-9- methyl-1,12-dihydro-14H- pyrano[3′,4′:6,7]indolizino[1,2- b]quinoline-3,14(4H)-dione | Compound 119 |
| 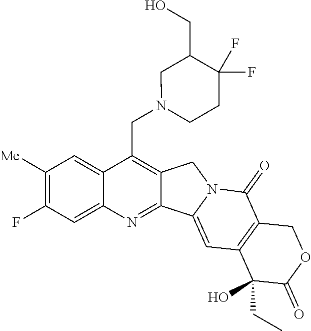 [SMILES: CC[C@@]1(O)C(=O)OCc2c1cc1n(c2=O)Cc2c-1nc1cc(F)c(C)cc1c2CN1CCC(F)(F)C(CO)C1] | (4S)-11-((4,4-difluoro-3- (hydroxymethyl)piperidin-1-yl)methyl)-4- ethyl-8-fluoro-4-hydroxy-9-methyl-1,12- dihydro-14H- pyrano[3′,4′:6,7]indolizino[1,2- b]quinoline-3,14(4H)-dione | Compound 120 |
| 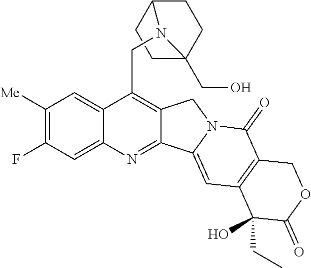 [SMILES: CC[C@@]1(O)C(=O)OCc2c1cc1n(c2=O)Cc2c-1nc1cc(F)c(C)cc1c2CN1C2CCC1(CO)CC2] | (4S)-11-((4,4-difluoro-3- (hydroxymethyl)piperidin-1-yl)methyl)-4- ethyl-8-fluoro-4-hydroxy-9-methyl-1,12- dihydro-14H- pyrano[3′,4′:6,7]indolizino[1,2- b]quinoline-3,14(4H)-dione | Compound 121 |
| 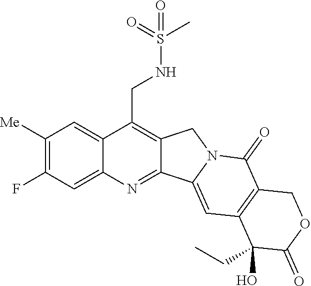 [SMILES: CC[C@@]1(O)C(=O)OCc2c1cc1n(c2=O)Cc2c-1nc1cc(F)c(C)cc1c2CNS(C)(=O)=O] | (S)-N-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11- yl)methyl)methanesulfonamide | Compound 122 |
| 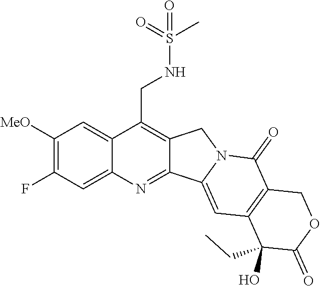 [SMILES: CC[C@@]1(O)C(=O)OCc2c1cc1n(c2=O)Cc2c-1nc1cc(F)c(OC)cc1c2CNS(C)(=O)=O] | (S)-N-((4-ethyl-8-fluoro-4-hydroxy-9- methoxy-3,14-dioxo-3,4,12,14-tetrahydro- 1H-pyrano[3′,4′:6,7]indolizino[1,2- b]quinolin-11- yl)methyl)methanesulfonamide | Compound 123 |
| 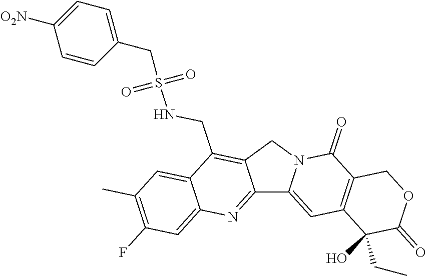 [SMILES: CC[C@@]1(O)C(=O)OCc2c1cc1n(c2=O)Cc2c-1nc1cc(F)c(C)cc1c2CNS(=O)(=O)Cc1ccc([N+](=O)[O-])cc1] | (S)-N-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11-yl)methyl)-1-(4- nitrophenyl)methanesulfonamide | Compound 124 |
| 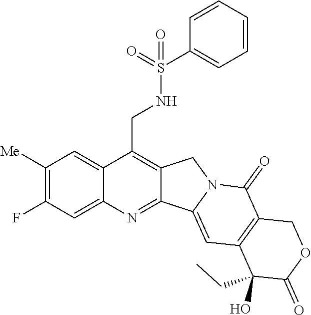 [SMILES: CC[C@@]1(O)C(=O)OCc2c1cc1n(c2=O)Cc2c-1nc1cc(F)c(C)cc1c2CNS(=O)(=O)c1ccccc1] | (S)-N-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11- yl)methyl)benzenesulfonamide | Compound 125 |
| 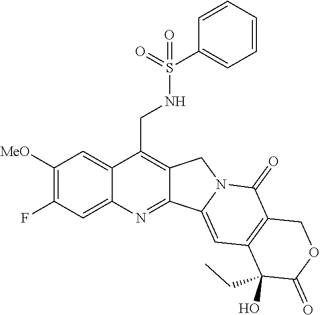 [SMILES: CC[C@@]1(O)C(=O)OCc2c1cc1n(c2=O)Cc2c-1nc1cc(F)c(OC)cc1c2CNS(=O)(=O)c1ccccc1] | (S)-N-((4-ethyl-8-fluoro-4-hydroxy-9- methoxy-3,14-dioxo-3,4,12,14-tetrahydro- 1H-pyrano[3′,4′:6,7]indolizino[1,2- b]quinolin-11- yl)methyl)benzenesulfonamide | Compound 126 |
| 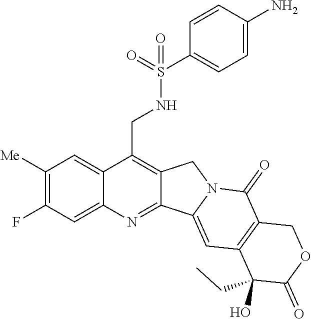 [SMILES: CC[C@@]1(O)C(=O)OCc2c1cc1n(c2=O)Cc2c-1nc1cc(F)c(C)cc1c2CNS(=O)(=O)c1ccc(N)cc1] | (S)-4-amino-N-((4-ethyl-8-fluoro-4- hydroxy-9-methyl-3,14-dioxo-3,4,12,14- tetrahydro-1H- pyrano[3′,4′:6,7]indolizino[1,2-b]quinolin- 11-yl)methyl)benzenesulfonamide | Compound 127 |
| 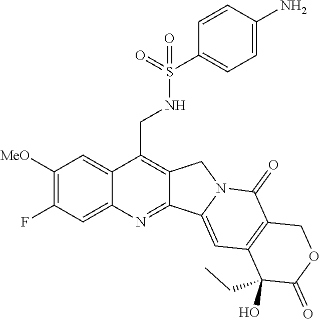 [SMILES: CC[C@@]1(O)C(=O)OCc2c1cc1n(c2=O)Cc2c-1nc1cc(F)c(OC)cc1c2CNS(=O)(=O)c1ccc(N)cc1] | (S)-4-amino-N-((4-ethyl-8-fluoro-4- hydroxy-9-methoxy-3,14-dioxo-3,4,12,14- tetrahydro-1H- pyrano[3′,4′:6,7]indolizino[1,2-b]quinolin- 11-yl)methyl)benzenesulfonamide | Compound 128 |
| 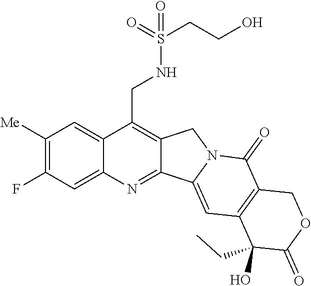 [SMILES: CC[C@@]1(O)C(=O)OCc2c1cc1n(c2=O)Cc2c-1nc1cc(F)c(C)cc1c2CNS(=O)(=O)CCO] | (S)-N-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11-yl)methyl)-2- hydroxyethane-1-sulfonamide | Compound 129 |
| 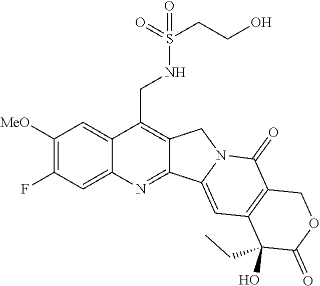 [SMILES: CC[C@@]1(O)C(=O)OCc2c1cc1n(c2=O)Cc2c-1nc1cc(F)c(OC)cc1c2CNS(=O)(=O)CCO] | (S)-N-((4-ethyl-8-fluoro-4-hydroxy-9- methoxy-3,14-dioxo-3,4,12,14-tetrahydro- 1H-pyrano[3′,4′:6,7]indolizino[1,2- b]quinolin-11-yl)methyl)-2- hydroxyethane-1-sulfonamide | Compound 130 |
| 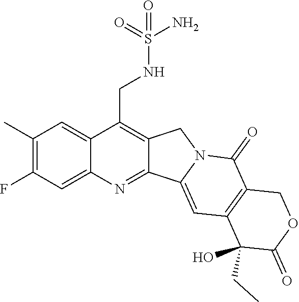 [SMILES: CC[C@@]1(O)C(=O)OCc2c1cc1n(c2=O)Cc2c-1nc1cc(F)c(C)cc1c2CNS(N)(=O)=O] | (S)-((4-ethyl-8-fluoro-4-hydroxy-9-methyl- 3,14-dioxo-3,4,12,14-tetrahydro-1H- pyrano[3′,4′:6,7]indolizino[1,2-b]quinolin- 11-yl)methyl)sulfamide | Compound 131 |
| 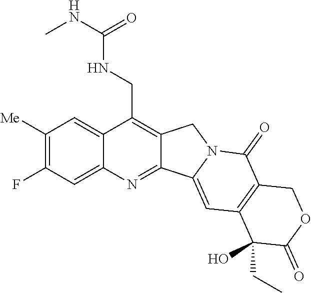 [SMILES: CC[C@@]1(O)C(=O)OCc2c1cc1n(c2=O)Cc2c-1nc1cc(F)c(C)cc1c2CNC(=O)NC] | (S)-1-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11-yl)methyl)-3-methylurea | Compound 132 |
| 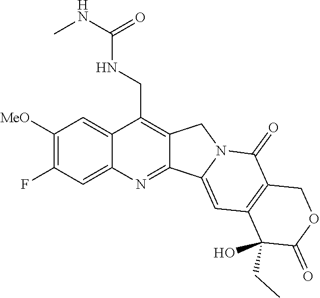 [SMILES: CC[C@@]1(O)C(=O)OCc2c1cc1n(c2=O)Cc2c-1nc1cc(F)c(OC)cc1c2CNC(=O)NC] | (S)-1-((4-ethyl-8-fluoro-4-hydroxy-9- methoxy-3,14-dioxo-3,4,12,14-tetrahydro- 1H-pyrano[3′,4′:6,7]indolizino[1,2- b]quinolin-11-yl)methyl)-3-methylurea | Compound 133 |
| 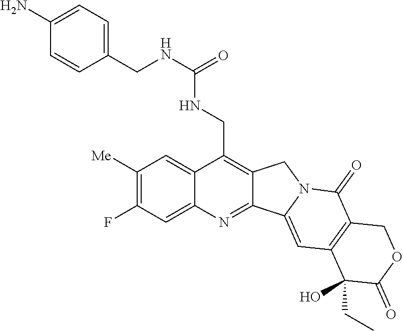 [SMILES: CC[C@@]1(O)C(=O)OCc2c1cc1n(c2=O)Cc2c-1nc1cc(F)c(C)cc1c2CNC(=O)NCc1ccc(N)cc1] | (S)-1-(4-aminobenzyl)-3-((4-ethyl-8- fluoro-4-hydroxy-9-methyl-3,14-dioxo- 3,4,12,14-tetrahydro-1H- pyrano[3′,4′:6,7]indolizino[1,2-b]quinolin- 11-yl)methyl)urea | Compound 134 |
| 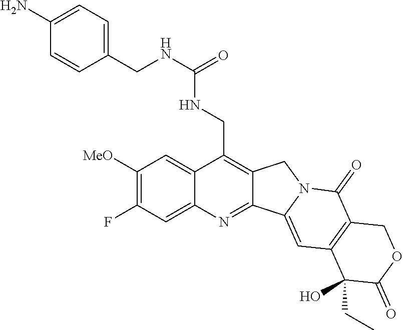 [SMILES: CC[C@@]1(O)C(=O)OCc2c1cc1n(c2=O)Cc2c-1nc1cc(F)c(OC)cc1c2CNC(=O)NCc1ccc(N)cc1] | (S)-1-(4-aminobenzyl)-3-((4-ethyl-8- fluoro-4-hydroxy-9-methoxy-3,14-dioxo- 3,4,12,14-tetrahydro-1H- pyrano[3′,4′:6,7]indolizino[1,2-b]quinolin- 11-yl)methyl)urea | Compound 135 |
| 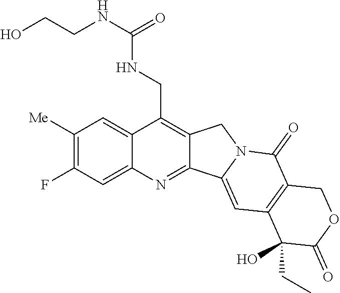 [SMILES: CC[C@@]1(O)C(=O)OCc2c1cc1n(c2=O)Cc2c-1nc1cc(F)c(C)cc1c2CNC(=O)NCCO] | (S)-1-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11-yl)methyl)-3-(2- hydroxyethyl)urea | Compound 136 |
| 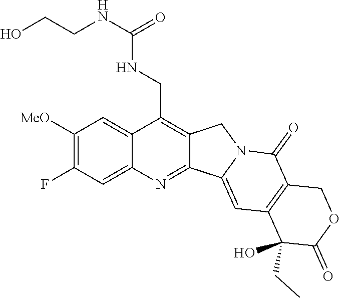 [SMILES: CC[C@@]1(O)C(=O)OCc2c1cc1n(c2=O)Cc2c-1nc1cc(F)c(OC)cc1c2CNC(=O)NCCO] | (S)-1-((4-ethyl-8-fluoro-4-hydroxy-9- methoxy-3,14-dioxo-3,4,12,14-tetrahydro- 1H-pyrano[3′,4′:6,7]indolizino[1,2- b]quinolin-11-yl)methyl)-3-(2- hydroxyethyl)urea | Compound 137 |
| 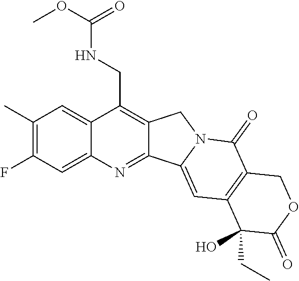 [SMILES: CC[C@@]1(O)C(=O)OCc2c1cc1n(c2=O)Cc2c-1nc1cc(F)c(C)cc1c2CNC(=O)OC] | methyl (S)-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11-yl)methyl)carbamate | Compound 138 |
| 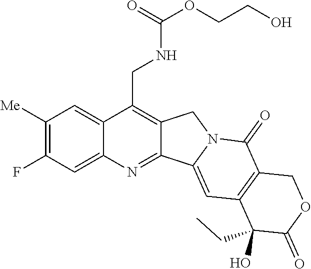 [SMILES: CC[C@@]1(O)C(=O)OCc2c1cc1n(c2=O)Cc2c-1nc1cc(F)c(C)cc1c2CNC(=O)OCCO] | 2-hydroxyethyl (S)-((4-ethyl-8-fluoro-4- hydroxy-9-methyl-3,14-dioxo-3,4,12,14- tetrahydro-1H- pyrano[3′,4′:6,7]indolizino[1,2-b]quinolin- 11-yl)methyl)carbamate | Compound 139 |

It is to be understood that reference to compounds of Formula (I) throughout this disclosure, includes in various embodiments, compounds of Formula (II) and Formula (III), as well as the individual compounds shown in Tables 5 and 6 to the same extent as if embodiments reciting each of these Formulae or compounds individually were specifically recited.

### Antibody-Drug Conjugates

As indicated above, the present disclosure relates to antibody-drug conjugates (ADCs) comprising an anti-GPC3 antibody construct conjugated to a camptothecin analogue having Formula (I). In certain embodiments, the ADC has Formula (X):

T-[L-(D)ₘ]ₙ (X)
wherein:
T is an anti-GPC3 antibody construct as described herein;
L is a linker;
D is a camptothecin analogue having Formula (I);
m is an integer between 1 and 4, and
n is an integer between 1 and 10.

In certain embodiments, in conjugates of Formula (X), m is between 1 and 2. In some embodiments, m is 1.

In some embodiments, in conjugates of Formula (X), n is between 1 and 8, for example, between 2 and 8. In some embodiments, n is between 4 and 8.

In certain embodiments, in conjugates of Formula (X), m is between 1 and 2, and n is between 2 and 8, or between 4 and 8. In some embodiments, in conjugates of Formula (X), m is 1, and n is between 2 and 8, or between 4 and 8.

As noted above and reflected by parameters m and n in Formula (X), the anti-GPC3 antibody construct, “T,” can be conjugated to more than one compound of Formula (I), “D.” Those skilled in the art will appreciate that, while any particular anti-GPC3 antibody construct T is conjugated to an integer number of compounds D, analysis of a preparation of the conjugate to determine the ratio of compound D to anti-GPC3 antibody construct T may give a non-integer result, reflecting a statistical average. This ratio of compound D to targeting moiety T may generally be referred to as the drug-to-antibody ratio, or “DAR.” Accordingly, conjugate preparations having non-integer DARs are intended to be encompassed by Formula (X).

In certain embodiments, in the conjugates of Formula (X), D is a compound of Formula Formula (II) or Formula (III). In certain embodiments, in the conjugates of Formula (X), D is a compound selected from the compounds shown in Tables 5 and 6. In certain embodiments, in the conjugates of Formula (X), D is Compound 139, Compound 140, Compound 141 or Compound 148. In some embodiments, in the conjugates of Formula (X), D is Compound 139 or Compound 141.

Certain embodiments of the present disclosure relate to ADCs having Formula (X), in which D is a compound of Formula (IV):

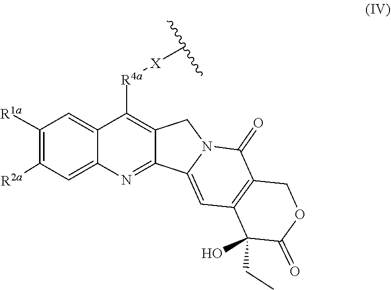

wherein:
   R^{1a} is selected from: —H, —CH₃, —CHF₂, —CF₃, —F, —Br, —Cl, —OH, —OCH₃, —OCF₃ and —NH₂;
   R^{2a} is selected from: —H, —CH₃, —CF₃, —F, —Br, —Cl, —OH, —OCH₃ and —OCF₃;
   X is —O—, —S— or —NH—, and R^{4a} is selected from:

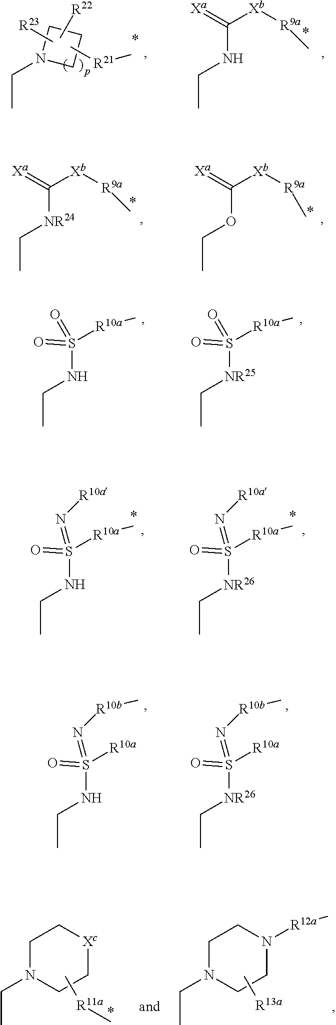

wherein * is the point of attachment to X, and wherein p is 1, 2, 3 or 4; or
X is O, and R^{4a}—X— is selected from;

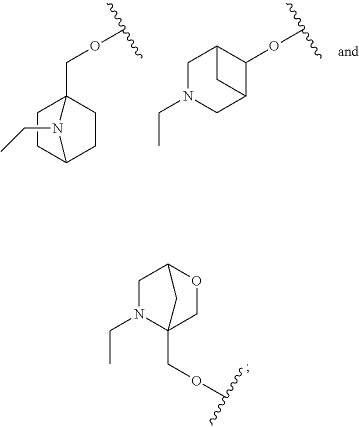 [SMILES: *OCC12CCC(CC1)N2CC ; *OC1C2CC1CN(CC)C2 ; *OCC12COC(CN1CC)C2]

R^{5a} is selected from: —C₁-C₆ alkyl, —C₃-C₈ cycloalkyl, -aryl, -heteroaryl and —(C₁-C₆ alkyl)-aryl;
R^{8a} is selected from: —C₁-C₆ alkyl, —C₃-C₈ cycloalkyl and —C₃-C₈ heterocycloalkyl;
each R^{9a} is independently selected from: —C₁-C₆ alkyl, —C₃-C₈ cycloalkyl, -aryl, -heteroaryl and —(C₁-C₆ alkyl)-aryl; or R^{9a} is absent and X^{b}═X;
each R^{10a} is independently selected from: —C₁-C₆ alkyl, —C₃-C₈ cycloalkyl, -aryl, -heteroaryl, —(C₁-C₆ alkyl)-aryl and

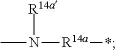

each R^{10a′}is independently selected from: —H, —C₁-C₆ alkyl, —C₃-C₈ cycloalkyl, -aryl,-heteroaryl and —(C₁-C₆ alkyl)-aryl;
each R^{10b} is independently selected from: —C₁-C₆ alkyl, —C₃-C₈ cycloalkyl, -aryl, -heteroaryl and —(C₁-C₆ alkyl)-aryl;
R^{11a} is absent or is —C₁-C₆ alkyl;
R^{12a} is selected from: —C₁-C₆ alkyl, —CO₂R^{8a}, -aryl, -heteroaryl, —(C₁-C₆ alkyl)-aryl, -S(O)₂R^{16a} and

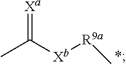

R^{13a} is selected from: —H and —C₁-C₆ alkyl;
R^{14a} is selected from: —C₁-C₆ alkyl, —C₃-C₈ cycloalkyl and —C₃-C₈ heterocycloalkyl;
R^{14a′} is selected from: H, —C₁-C₆ alkyl, —C₃-C₈ cycloalkyl and —C₃-C₈ heterocycloalkyl;
R^{16a} is selected from: —C₁-C₆ alkyl, —C₃-C₈ cycloalkyl, -aryl, -heteroaryl and —(C₁-C₆ alkyl)-aryl;
R²¹ is selected from: —C₁-C₆ alkyl, —C₃-C₈ cycloalkyl and —(C₁-C₆ alkyl)-O—R^{5a};
R²² and R²³ are each independently selected from: —H, -halogen, —C₁-C₆ alkyl and —C₃-C₈ cycloalkyl;
R²⁴, R²⁵ and R²⁶ are each —C₁-C₆ alkyl;
X^{a} and X^{b} are each independently selected from: NH, O and S; X^{c} is selected from: 0, S and S(O)₂, and

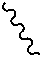

denotes the point of attachment to linker, L.

In some embodiments, in compounds of Formula (IV), R^{1a} is selected from: —CH₃, —CF₃,-OCH₃, —OCF₃ and —NH₂.

In some embodiments, in compounds of Formula (IV), R^{1a} is selected from: —CH₃, —CF₃,-OCH₃ and —OCF₃.

In some embodiments, in compounds of Formula (IV), R^{1a} is selected from: —CH₃, —OCH₃ and NH₂.

In some embodiments, in compounds of Formula (IV), R^{1a} is selected from: —CH₃ and —OCH₃.

In some embodiments, in compounds of Formula (IV), R^{2a} is selected from: —H, —CH₃, —CF₃, —F, —Cl, —OCH₃ and —OCF₃.

In some embodiments, in compounds of Formula (IV), R^{2a} is selected from: —H, —F and —Cl.

In some embodiments, in compounds of Formula (IV), R^{2a} is —F.

In some embodiments, in compounds of Formula (IV), X is —O—, —S— or —NH—, and R^{4a} is selected from:

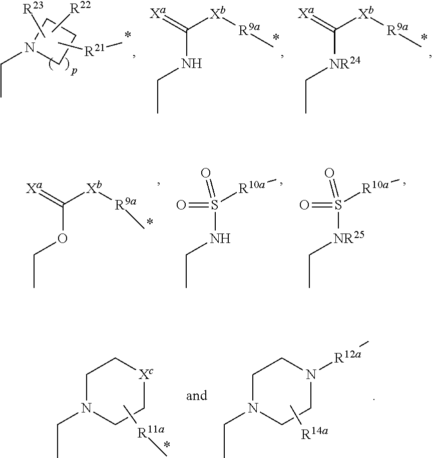

In some embodiments, in compounds of Formula (IV), X is —O— or —NH—.

In some embodiments, in compounds of Formula (IV), each R^{9a} is independently selected from: —C₁-C₆ alkyl, —C₃-C₈ cycloalkyl, -aryl and —(C₁-C₆ alkyl)-aryl.

In some embodiments, in compounds of Formula (IV), each R^{9a} is independently selected from: —C₁-C₆ alkyl and —(C₁-C₆ alkyl)-aryl.

In some embodiments, in compounds of Formula (IV), each R^{10a} is independently selected from: —C₁-C₆ alkyl, —C₃-C₈ cycloalkyl, -aryl, —(C₁-C₆ alkyl)-aryl and

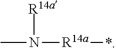

In some embodiments, in compounds of Formula (IV), each R^{10a} is independently selected from: —C₁-C₆ alkyl, -aryl, —(C₁-C₆ alkyl)-aryl and

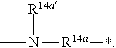

In some embodiments, in compounds of Formula (IV), R^{12a} is selected from: —C₁-C₆ alkyl,-aryl, —(C₁-C₆ alkyl)-aryl and —S(O)₂R¹⁶.

In some embodiments, in compounds of Formula (IV), R^{13a} is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (IV), R^{14a′} is selected from: H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (IV), R^{16a} is selected from: -aryl, -heteroaryl and —(C₁-C₆ alkyl)-aryl.

In some embodiments, in compounds of Formula (IV), R²² and R²³ are each independently selected from: —H, -halogen, unsubstituted —C₁-C₆ alkyl, —C₁-C₆ haloalkyl, —C₁-C₆ aminoalkyl, —C₁-C₆ hydroxyalkyl and —C₃-C₈ cycloalkyl.

In some embodiments, in compounds of Formula (IV), X^{a} and X^{b} are each independently selected from: NH and O.

In some embodiments, in compounds of Formula (IV), X^{a} and X^{b} are each O.

In some embodiments, in compounds of Formula (IV), X is O; R^{4a} is

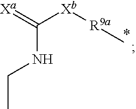

X^{a} and X^{b} are each O, and R^{9a} is —C₁-C₆ alkyl.

In some embodiments, in compounds of Formula (IV), R^{1a} is —CH₃ or -OCH₃; X is O; R^{4a} is

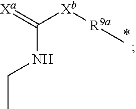

X^{a} and X^{b} are each O; and R^{9a} is —C₁-C₆ alkyl.

In some embodiments, in compounds of Formula (IV), R^{1a} is —CH₃ or -OCH₃; R^{2a} is H or F; X is O; R^{4a} is

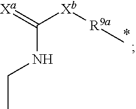

X^{a} and X^{b} are each 0; and R^{9a} is —C₁-C₆ alkyl.

Other combinations of any of the foregoing embodiments for compounds of Formula (IV) are also contemplated and each combination forms a separate embodiment for the purposes of the present disclosure.

Certain embodiments of the present disclosure relate to ADCs having Formula (X), in which D is a compound of Formula (V):

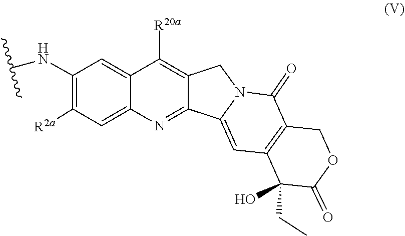

wherein:
   R^{2a} is selected from: —CH₃, —CF₃, —F, —Br, —Cl, —OH, —OCH₃ and —OCF₃;
   R^{20a} is selected from: —H, —C₁-C₆ alkyl, —C₃-C₈ cycloalkyl, —(C₁-C₆ alkyl)-O—R⁵,

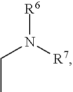

—CO₂R⁸, -aryl, -heteroaryl, —(C₁-C₆ alkyl)-aryl,

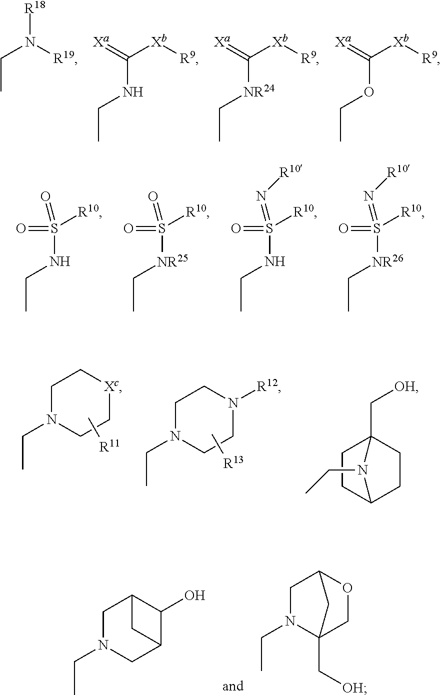

R⁵ is selected from: —H, —C₁-C₆ alkyl, —C₃-C₈ cycloalkyl, -aryl, -heteroaryl and —(C₁-C₆ alkyl)-aryl;
R⁶ and R⁷ are each independently selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, -aryl, -heteroaryl, —(C₁-C₆ alkyl)-aryl and —NR¹⁴R^{14′};
each R^{10′}is independently selected from: —H, —C₁-C₆ alkyl, —C₃-C₈ cycloalkyl, -aryl, -heteroaryl and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl,-S(O)₂R¹⁶ and

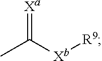

R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁷ is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R¹⁸ and R¹⁹ taken together with the N atom to which they are bonded form a 4-, 5-, 6-, or 7-membered ring having 0 to 3 substituents selected from: halogen, —C₁-C₆ alkyl, —C₃-C₈ cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, 0 and S;
X^{c} is selected from: 0, S and S(O)₂, and

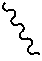

denotes the point of attachment to linker, L.

In some embodiments, in compounds of Formula (V), R^{2a} is selected from: —CH₃, —CF₃, —F, —Cl, —OCH₃ and —OCF₃.

In some embodiments, in compounds of Formula (V), R^{2a} is selected from: —CF₃, —F, —Cl and —OCH₃.

In some embodiments, in compounds of Formula (V), R^{2a} is F.

In some embodiments, in compounds of Formula (V), R^{20a} is selected from: —H, —C₁-C₆ alkyl, —C₃-C₈ cycloalkyl, —(C₁-C₆ alkyl)-O—R⁵,

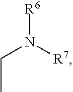

—CO₂R⁸, -aryl, -heteroaryl, —(C₁-C₆ alkyl)-aryl,

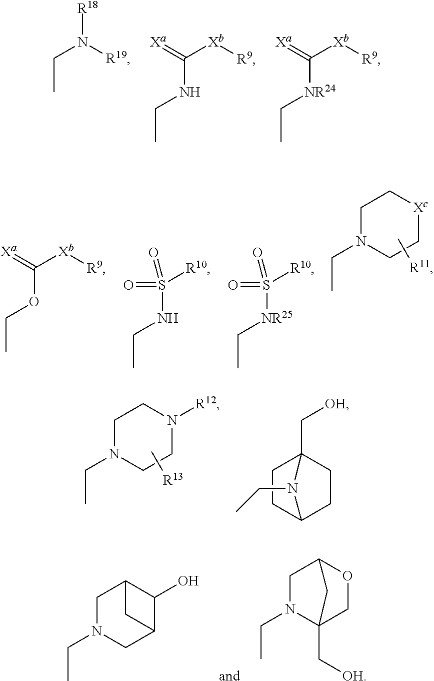

In some embodiments, in compounds of Formula (V), R^{20a} is selected from: —H, —C₁-C₆ alkyl, —(C₁-C₆ alkyl)-O—R⁵,

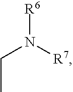

—(C₁-C₆ alkyl)-aryl,

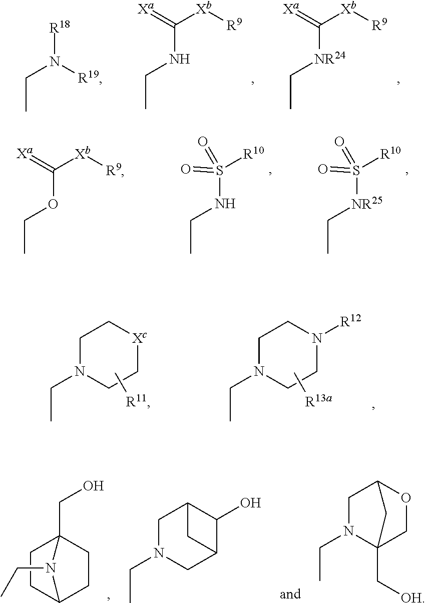

In some embodiments, in compounds of Formula (V), R^{20a} is selected from: —H, —C₁-C₆ alkyl, —(C₁-C₆ alkyl)-O—R⁵,

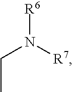

—(C₁-C₆ alkyl)-aryl,

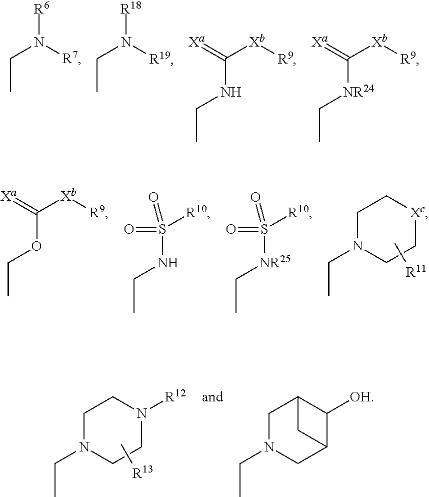

In some embodiments, in compounds of Formula (V), R^{20a} is selected from: —H, —C₁-C₆ alkyl, —(C₁-C₆ alkyl)-O—R⁵,

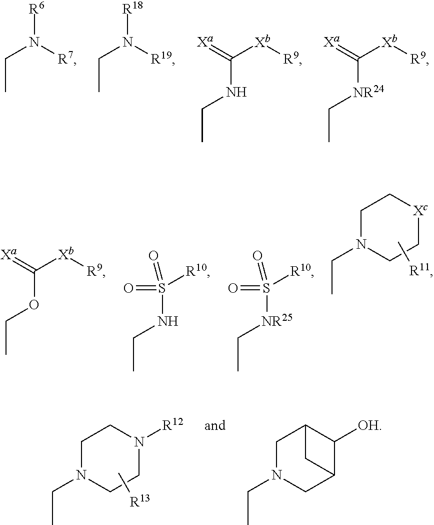

In some embodiments, in compounds of Formula (V), R^{20a} is selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵,

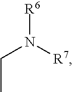

—CO₂R⁸, unsubstituted-aryl, -aminoaryl, -heteroaryl, —(C₁-C₆ alkyl)-aminoaryl,

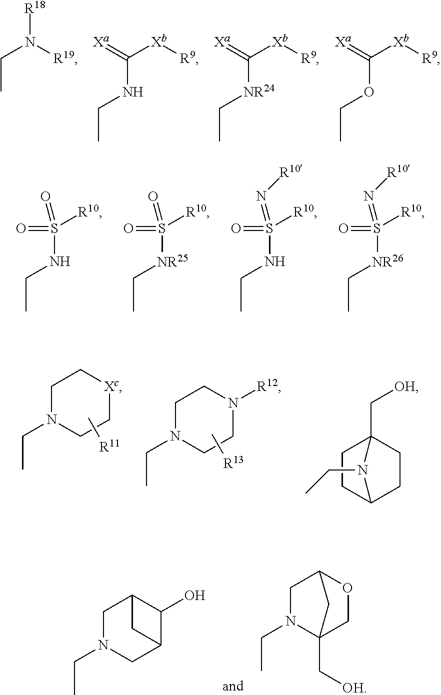

In some embodiments, in compounds of Formula (V), R⁶ and R⁷ are each independently selected from: —H, —C₁-C₆ alkyl, —C₃-C₈ cycloalkyl and —C(O)R¹⁷.

In some embodiments, in compounds of Formula (V), R⁶ is H, and R⁷ is selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷.

In some embodiments, in compounds of Formula (V), R⁶ is H, and R⁷ is selected from: —H, —C₁-C₆ alkyl, —C₃-C₈ cycloalkyl and —C(O)R¹⁷.

In some embodiments, in compounds of Formula (V), R⁶ and R⁷ are each independently selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵, —C₃-C₈ heterocycloalkyl and —C(O)R¹⁷.

In some embodiments, in compounds of Formula (V), R⁸ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (V), each R⁹ is independently selected from: —C₁-C₆ alkyl, —C₃-C₈ cycloalkyl, -aryl and —(C₁-C₆ alkyl)-aryl.

In some embodiments, in compounds of Formula (V), each R⁹ is independently selected from: —C₁-C₆ alkyl and —(C₁-C₆ alkyl)-aryl.

In some embodiments, in compounds of Formula (V), each R⁹ is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted-aryl, -aminoaryl, -heteroaryl and —(C₁-C₆ alkyl)-aminoaryl.

In some embodiments, in compounds of Formula (V), each R¹⁰ is independently selected from: —C₁-C₆ alkyl, —C₃-C₈ cycloalkyl, —NR¹⁴R^{14′}, -aryl and —(C₁-C₆ alkyl)-aryl.

In some embodiments, in compounds of Formula (V), each R¹⁰ is independently selected from: —C₁-C₆ alkyl, —NR¹⁴R^{14′}, -aryl and —(C₁-C₆ alkyl)-aryl.

In some embodiments, in compounds of Formula (V), R¹¹ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (V), R¹² is selected from: —H, —C₁-C₆ alkyl, -aryl, —(C₁-C₆ alkyl)-aryl and —S(O)₂R¹⁶.

In some embodiments, in compounds of Formula (V), R¹² is selected from: —H, unsubstituted —C₁-C₆ alkyl, —C₁-C₆ haloalkyl, —C₁-C₆ hydroxyalkyl, —C₁-C₆ aminoalkyl, —CO₂R⁸, unsubstituted-aryl, -aminoaryl, -heteroaryl, —(C₁-C₆ alkyl)-aminoaryl, —S(O)₂R¹⁶ and

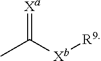

In some embodiments, in compounds of Formula (V), R¹³ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (V), R¹⁴ and R^{14′} are each independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (V), R¹⁶ is selected from: -aryl, -heteroaryl and —(C₁-C₆ alkyl)-aryl.

In some embodiments, in compounds of Formula (V), R¹⁶ is selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted-aryl, -aminoaryl, -heteroaryl and —(C₁-C₆ alkyl)-aminoaryl.

In some embodiments, in compounds of Formula (V), R¹⁷ is selected from: unsubstituted —C₁-C₆ alkyl, —C₃-C₈ cycloalkyl, —C₃-C₈ heterocycloalkyl, —(C₁-C₆ alkyl)-C₃-C₈ heterocycloalkyl, unsubstituted-aryl, -hydroxyaryl, -aminoaryl, -heteroaryl and —(C₁-C₆ alkyl)-aminoaryl.

In some embodiments, in compounds of Formula (V), R¹⁸ and R¹⁹ taken together with the N atom to which they are bonded form a 4-, 5-, 6-, or 7-membered ring having 0 to 3 substituents selected from: halogen, unsubstituted —C₁-C₆ alkyl, —C₁-C₆ haloalkyl, —C₁-C₆ aminoalkyl, —C₁-C₆ hydroxyalkyl, —C₃-C₈ cycloalkyl and —(C₁-C₆ alkyl)-O—R^{S}.

In some embodiments, in compounds of Formula (V), R¹⁷ is —C₁-C₆ alkyl.

In some embodiments, in compounds of Formula (V), X^{a} and X^{b} are each independently selected from: NH and O.

In some embodiments, in compounds of Formula (V), X^{a} and X^{b} are each O.

In some embodiments, in compounds of Formula (V), R^{20a} is —(C₁-C₆ alkyl)-O—R⁵.

In some embodiments, in compounds of Formula (V), R^{20a} is —(C₁-C₆ alkyl)-O—R⁵, and R⁵ is H.

In some embodiments, in compounds of Formula (V), R^{2a} is F; R^{20a} is —(C₁-C₆ alkyl)-O—R⁵, and R⁵ is H.

Other combinations of any of the foregoing embodiments for compounds of Formula (V) are also contemplated and each combination forms a separate embodiment for the purposes of the present disclosure.

Certain embodiments of the present disclosure relate to ADCs having Formula (X), in which D is a compound of Formula (VI):

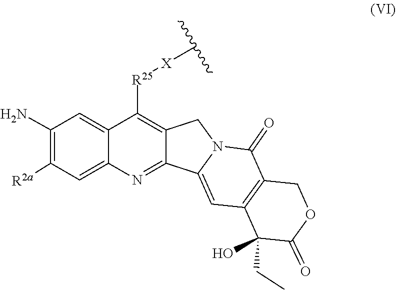

wherein:
   R^{2a} is selected from: —H, —CH₃, —CF₃, —F, —Br, —Cl, —OH, —OCH₃ and —OCF₃;
   X is —O—, —S— or —NH—, and R²5 is selected from: —C₁-C₆ alkyl, —(C₁-C₆ alkyl)-O—R^{5a}, —CO₂R^{8a}, -aryl, -heteroaryl, —(C₁-C₆ alkyl)-aryl,

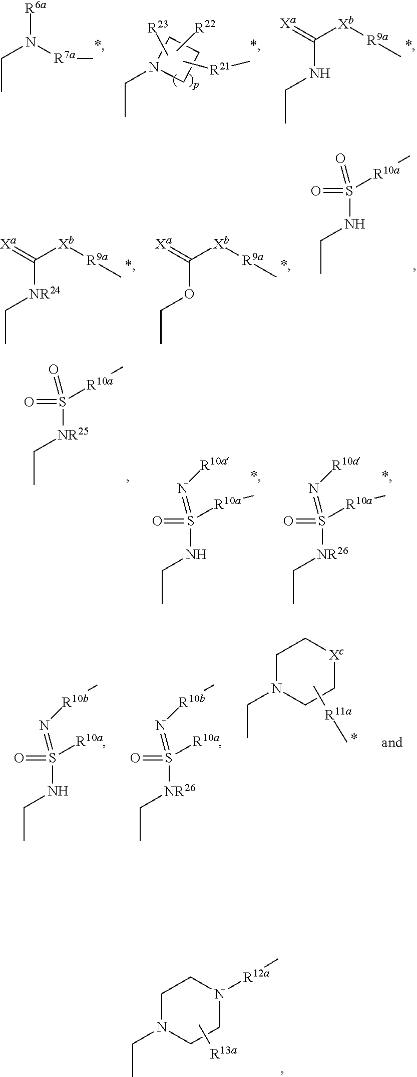

wherein * is the point of attachment to X, and wherein p is 1, 2, 3 or 4; or
X is O, and R²⁵—X— is selected from:

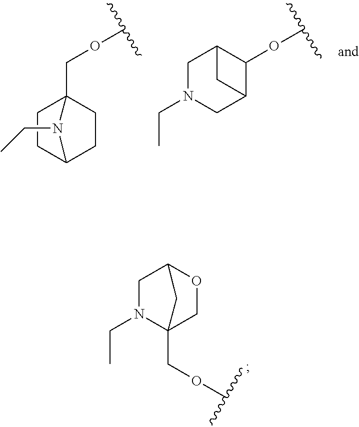 [SMILES: *OCC12CCC(CC1)N2CC ; *OC1C2CC1CN(CC)C2 ; *OCC12COC(CN1CC)C2]

R^{5a} is selected from: —C₁-C₆ alkyl, —C₃-C₈ cycloalkyl, -aryl, -heteroaryl and —(C₁-C₆ alkyl)-aryl;
R^{6a} is selected from: —H, —C₁-C₆ alkyl, —C₃-C₈ cycloalkyl and —C₃-C₈ heterocycloalkyl;
R^{7a} is selected from: —C₁-C₆ alkyl, —C₃-C₈ cycloalkyl, —(C₁-C₆ alkyl)-O—R^{5a}, —C₃-C₈ heterocycloalkyl and —C(O)R^{17a};
R^{8a} is selected from: —C₁-C₆ alkyl, —C₃-C₈ cycloalkyl and —C₃-C₈ heterocycloalkyl;
each R^{9a} is independently selected from: —C₁-C₆ alkyl, —C₃-C₈ cycloalkyl, -aryl, -heteroaryl and —(C₁-C₆ alkyl)-aryl; or R^{9a} is absent and X^{b}═X;
each R^{10a} is independently selected from: —C₁-C₆ alkyl, —C₃-C₈ cycloalkyl, -aryl, -heteroaryl, —(C₁-C₆ alkyl)-aryl and

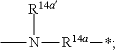

each R^{10a′} is independently selected from: —H, —C₁-C₆ alkyl, —C₃-C₈ cycloalkyl, -aryl, -heteroaryl and —(C₁-C₆ alkyl)-aryl;
each R^{10b} is independently selected from: —C₁-C₆ alkyl, —C₃-C₈ cycloalkyl, -aryl, -heteroaryl and —(C₁-C₆ alkyl)-aryl;
   R^{11a} is absent or is —C₁-C₆ alkyl;
   R^{12a} is selected from: —C₁-C₆ alkyl, —CO₂R^{8a}, -aryl, -heteroaryl, —(C₁-C₆ alkyl)-aryl, -S(O)₂R^{16a} and

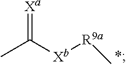

R^{13a} is selected from: —H and —C₁-C₆ alkyl;
R^{14a} is selected from: —C₁-C₆ alkyl, —C₃-C₈ cycloalkyl and —C₃-C₈ heterocycloalkyl;
R^{14a′}is selected from: H, —C₁-C₆ alkyl, —C₃-C₈ cycloalkyl and —C₃-C₈ heterocycloalkyl;
R^{16a} is selected from: —C₁-C₆ alkyl, —C₃-C₈ cycloalkyl, -aryl, -heteroaryl and —(C₁-C₆ alkyl)-aryl;
R^{17a} is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R²¹ is selected from: —C₁-C₆ alkyl, —C₃-C₈ cycloalkyl and —(C₁-C₆ alkyl)-O—R^{5a};
R²² and R²³ are each independently selected from: —H, -halogen, —C₁-C₆ alkyl and —C₃-C₈ cycloalkyl;
R²⁴, R²⁵ and R²⁶ are each —C₁-C₆ alkyl;
X^{a} and X^{b} are each independently selected from: NH, 0 and S;
X^{c} is selected from: 0, S and S(O)₂, and

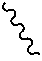

denotes the point of attachment to linker, L.

In some embodiments, in compounds of Formula (VI), R^{2a} is selected from: —CH₃, —CF₃, —F, —Br, —Cl, —OH, —OCH₃ and —OCF₃.

In some embodiments, in compounds of Formula (VI), R^{2a} is selected from: —CH₃, —CF₃, —F, —Cl, —OCH₃ and —OCF₃.

In some embodiments, in compounds of Formula (VI), R^{2a} is selected from: F and Cl.

In some embodiments, in compounds of Formula (VI), R^{2a} is F.

In some embodiments, in compounds of Formula (VI), X is —O—, —S— or —NH—, and R²⁵ is selected from: —C₁-C₆ alkyl, —(C₁-C₆ alkyl)-O—R^{5a}, —(C₁-C₆ alkyl)-aryl,

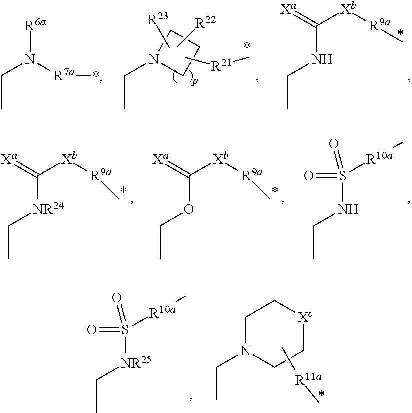

and

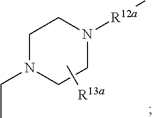

or X is O, and R²⁵—X— is selected from:

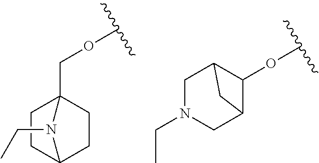 [SMILES: *OCC12CCC(CC1)N2CC ; *OC1C2CC1CN(CC)C2]

and

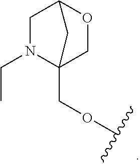 [SMILES: *OCC12COC(CN1CC)C2]

In some embodiments, in compounds of Formula (VI), X is —O—, —S— or —NH—, and R²⁵ is selected from: —C₁-C₆ alkyl, —(C₁-C₆ alkyl)-O—R^{5a}, —(C₁-C₆ alkyl)-aryl,

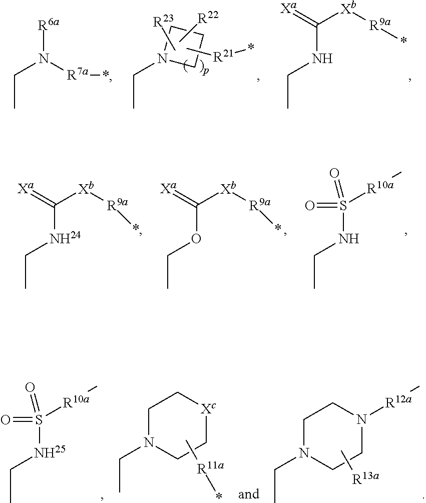

In some embodiments, in compounds of Formula (VI), X is —O—, —S— or —NH—, and R²⁵ is selected from: —C₁-C₆ alkyl, —(C₁-C₆ alkyl)-O—R^{5a}

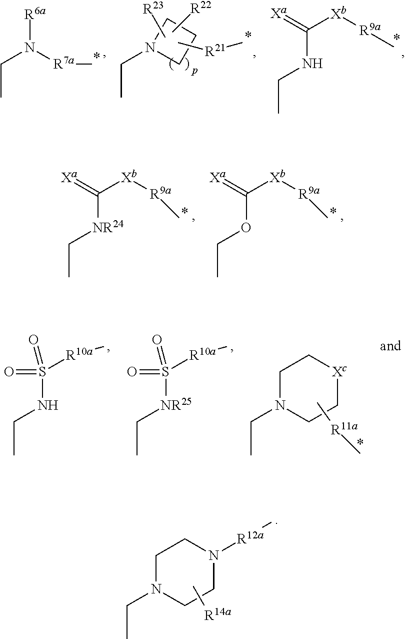

In some embodiments, in compounds of Formula (VI), X is —O—, —S— or —NH—, and R²⁵ is selected from:

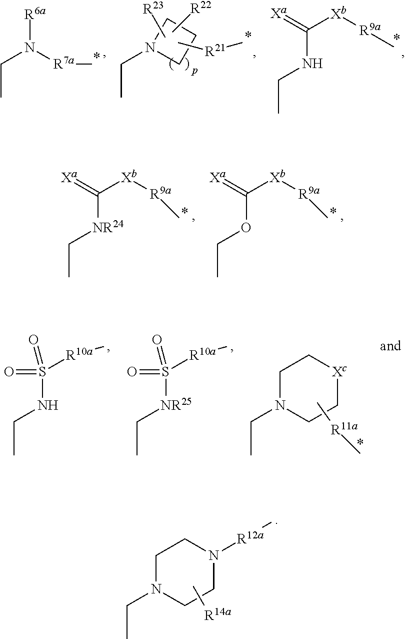

In some embodiments, in compounds of Formula (VI), X is —O— or —NH—.

In some embodiments, in compounds of Formula (VI), R^{6a} is H.

In some embodiments, in compounds of Formula (VI), R^{6a} is selected from: —H, unsubstituted —C₁-C₆ alkyl, —C₁-C₆ haloalkyl, —C₁-C₆ hydroxyalkyl, —C₃-C₈ cycloalkyl and —C₃-C₈ heterocycloalkyl.

In some embodiments, in compounds of Formula (VI), R^{7a} is selected from: —C₁-C₆ alkyl, —C₃-C₈ cycloalkyl and —C(O)R^{17a}.

In some embodiments, in compounds of Formula (VI), each R^{9a} is independently selected from: —C₁-C₆ alkyl, —C₃-C₈ cycloalkyl, -aryl and —(C₁-C₆ alkyl)-aryl.

In some embodiments, in compounds of Formula (VI), each R^{9a} is independently selected from: —C₁-C₆ alkyl and —(C₁-C₆ alkyl)-aryl.

In some embodiments, in compounds of Formula (VI), each R^{10a} is independently selected from: —C₁-C₆ alkyl, —C₃-C₈ cycloalkyl, -aryl, —(C₁-C₆ alkyl)-aryl and

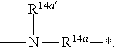

In some embodiments, in compounds of Formula (VI), each R^{10a} is independently selected from: —C₁-C₆ alkyl, -aryl, —(C₁-C₆ alkyl)-aryl and

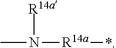

In some embodiments, in compounds of Formula (VI), R^{12a} is selected from: —C₁-C₆ alkyl, -aryl, —(C₁-C₆ alkyl)-aryl and —S(O)₂R^{16a}.

In some embodiments, in compounds of Formula (VI), R^{13a} is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (VI), R^{14a′} is selected from: H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (VI), R^{16a} is selected from: -aryl, -heteroaryl and —(C₁-C₆ alkyl)-aryl.

In some embodiments, in compounds of Formula (VI), R^{17a} is —C₁-C₆ alkyl.

In some embodiments, in compounds of Formula (VI), R²² and R²³ are each independently selected from: —H, -halogen, unsubstituted —C₁-C₆ alkyl, —C₁-C₆ haloalkyl, —C₁-C₆ hydroxyalkyl, —C₁-C₆ aminoalkyl and —C₃-C₈ cycloalkyl.

In some embodiments, in compounds of Formula (VI), X^{a} and X^{b} are each independently selected from: NH and O.

In some embodiments, in compounds of Formula (VI), X^{a} and X^{b} are each O.

In some embodiments, in compounds of Formula (VI), X is O, and R²⁵ is —C₁-C₆ alkyl.

In some embodiments, in compounds of Formula (VI), R^{2a} is F; X is O, and R²⁵ is —C₁-C₆ alkyl.

Other combinations of any of the foregoing embodiments for compounds of Formula (VI) are also contemplated and each combination forms a separate embodiment for the purposes of the present disclosure.

In certain embodiments, each alkyl, cycloalkyl, heterocycloalkyl, aryl and heteroaryl group as defined in any one of Formulae (IV), (V) or (VI) is optionally substituted with one or more substituents selected from: halogen, acyl, acyloxy, alkoxy, carboxy, hydroxy, amino, amido, nitro, cyano, azido, alkylthio, thio, sulfonyl, sulfonamido, alkyl, cycloalkyl, heterocycloalkyl, aryl and heteroaryl. In some embodiments, each alkyl, cycloalkyl, heterocycloalkyl, aryl and heteroaryl group as defined in any one of Formulae (IV), (V) or (VI) is optionally substituted with one or more substituents selected from: halogen, acyl, acyloxy, alkoxy, carboxy, hydroxy, amino, amido, nitro, cyano, azido, alkylthio, thio, sulfonyl and sulfonamido.

In certain embodiments, in ADCs having Formula (X), D is a compound of Formula (IV), in which R^{1a} is —CH₃, and R^{2a} is F. In some embodiments, in ADCs having Formula (X), D is a compound of Formula (IV), in which R^{1a} is —CH₃; R^{2a} is F; X is —O—; R^{4a} is

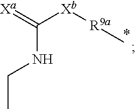

R^{9a} is —C₁-C₆ alkyl, and X^{a} and X^{b} are each O.

In certain embodiments, in ADCs having Formula (X), D is a compound of Formula (V), in which R^{2a} is F, and R^{20a} is H, —(C₁-C₆)-O—R^{S} or

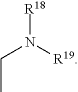

In some embodiments, in ADCs having Formula (X), D is a compound of Formula (V), in which R^{2a} is F; R²0a is H, —(C₁-C₆)-O—R⁵ or

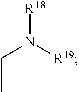

R⁵ is H, and R¹⁸ and R¹⁹ taken together with the N atom to which they are bonded form an unsubstituted 4-, 5-, 6-, or 7-membered ring. In some embodiments, in ADCs having Formula (X), D is a compound of Formula (V), in which R^{2a} is F; R²0a is —(C₁-C₆)-O—R⁵, and R⁵ is H.

In certain embodiments, in ADCs having Formula (X), D is a compound of Formula (VI), in which R^{2a} is F; X is —O—, and R²⁵ is —C₁-C₆ alkyl.

### Linker, L

The conjugates of Formula (X) include a linker, L, which is a bifunctional or multifunctional moiety capable of linking one or more camptothecin analogues, D, to the anti-GPC3 antibody construct, T. A bifunctional (or monovalent) linker, L, links a single compound D to a single site on the anti-GPC3 antibody construct, T, whereas a multifunctional (or polyvalent) linker, L, links more than one compound, D, to a single site on the anti-GPC3 antibody construct, T. A linker that links one compound, D, to more than one site on the anti-GPC3 antibody construct, T, may also be considered to be multifunctional.

Linker, L, includes a functional group capable of reacting with the target group or groups on the anti-GPC3 antibody construct, T, and at least one functional group capable of reacting with a target group on the camptothecin analogue, D. Suitable functional groups are known in the art and include those described, for example, in Bioconjugate Techniques (G. T. Hermanson, 2013, Academic Press). Groups on the anti-GPC3 antibody construct, T, and the camptothecin analogue, D, that may serve as target groups for linker attachment include, but are not limited to, thiol, hydroxyl, carboxyl, amine, aldehyde and ketone groups.

Non-limiting examples of functional groups capable of reacting with thiols include maleimide, haloacetamide, haloacetyl, activated esters (such as succinimide esters, 4-nitrophenyl esters, pentafluorophenyl esters and tetrafluorophenyl esters), anhydrides, acid chlorides, sulfonyl chlorides, isocyanates and isothiocyanates. Also useful in this context are “self-stabilizing” maleimides as described in Lyon et al., 2014, Nat. Biotechnol., 32:1059-1062.

Non-limiting examples of functional groups capable of reacting with amines include activated esters (such as N-hydroxysuccinamide (NHS) esters and sulfo-NHS esters), imido esters (such as Traut's reagent), isothiocyanates, aldehydes and acid anhydrides (such as diethylenetriaminepentaacetic anhydride (DTPA)). Other examples include the use of succinimido-1,1,3,3-tetra-methyluronium tetrafluoroborate (TSTU) or benzotriazol-1-yl-oxytripyrrolidinophosphonium hexafluorophosphate (PyBOP) to convert a carboxyl group to an activated ester, which may then be reacted with an amine.

Non-limiting examples of functional groups capable of reacting with an electrophilic group such as an aldehyde or ketone carbonyl group include hydrazide, oxime, amino, hydrazine, thiosemicarbazone, hydrazine carboxylate and arylhydrazide.

In certain embodiments, linker, L, may include a functional group that allows for bridging of two interchain cysteines on the anti-GPC3 antibody construct, such as a ThioBridge™ linker (Badescu et al., 2014, Bioconjug. Chem. 25:1124-1136), a dithiomaleimide (DTM) linker (Behrens et al., 2015, Mol. Pharm. 12:3986-3998), a dithioaryl(TCEP)pyridazinedione-based linker (Lee et al., 2016, Chem. Sci., 7:799-802) or a dibromopyridazinedione-based linker (Maruani et al., 2015, Nat. Commun., 6:6645).

Alternatively, the anti-GPC3 antibody construct, T, may be modified to include a non-natural reactive group, such as an azide, that allows for conjugation to the linker via a complementary reactive group on the linker. For example, conjugation of the linker to the anti-GPC3 antibody construct may make use of click chemistry reactions (see, for example, Chio & Bane, 2020, Methods Mol. Biol., 2078:83-97), such as the azide-alkyne cycloaddition (AAC) reaction, which has been used successfully in the development of antibody-drug conjugates. The AAC reaction may be a copper-catalyzed AAC (CuAAC) reaction, which involves coupling of an azide with a linear alkyne, or a strain-promoted AAC (SPAAC) reaction, which involves coupling of an azide with a cyclooctyne.

Linker, L, may be a cleavable or a non-cleavable linker. A cleavable linker is a linker that is susceptible to cleavage under specific conditions, for example, intracellular conditions (such as in an endosome or lysosome) or within the vicinity of a target cell (such as in the tumor microenvironment). Examples include linkers that are protease-sensitive, acid-sensitive or reduction-sensitive. Non-cleavable linkers by contrast, rely on the degradation of the antibody in the cell, which typically results in the release of an amino acid-linker-drug moiety.

Examples of cleavable linkers include, for example, linkers comprising an amino acid sequence that is a cleavage recognition sequence for a protease. Many such cleavage recognition sequences are known in the art. For conjugates that are not intended to be internalized by a cell, for example, an amino acid sequence that is recognized and cleaved by a protease present in the extracellular matrix in the vicinity of a target cell, such as a cancer cell, may be employed. Examples of extracellular tumor-associated proteases include, for example, plasmin, matrix metalloproteases (MMPs), elastase and kallikrein-related peptidases.

For conjugates intended to be internalized by a cell, linker, L, may comprise an amino acid sequence that is recognized and cleaved by an endosomal or lysosomal protease. Examples of such proteases include, for example, cathepsins B, C, D, H, L and S, and legumain.

Cleavage recognition sequences may be, for example, dipeptides, tripeptides or tetrapeptides. Non-limiting examples of dipeptide recognition sequences that may be included in cleavable linkers include, but are not limited to, Ala-(D)Asp, Ala-Lys, Ala-Phe, Asn-Lys, Asn-(D)Lys, Asp-Val, His-Val, Ile-Cit, Ile-Pro, Ile-Val, Leu-Cit, Me3Lys-Pro, Met-Lys, Met-(D)Lys, NorVal-(D)Asp, Phe-Arg, Phe-Cit, Phe-Lys, PhenylGly-(D)Lys, Pro-(D)Lys, Trp-Cit, Val-Ala, Val-(D)Asp, Val-Cit, Val-Gly, Val-Gln and Val-Lys. Examples of tri- and tetrapeptide cleavage sequences include, but are not limited to, Ala-Ala-Asn, Ala-Val-Cit, (D)Ala-Phe-Lys, Asp-Val-Ala, Asp-Val-Cit, Gly-Cit-Val, Lys-Val-Ala, Lys-Val-Cit, Met-Cit-Val, (D)Phe-Phe-Lys, Asn-Pro-Val, Ala-Leu-Ala-Leu, Gly-Phe-Leu-Gly, Gly-Gly-Phe-Gly and Gly-Phe-Gly-Gly.

Additional examples of cleavable linkers include disulfide-containing linkers such as N-succinimydyl-4-(2-pyridyldithio) butanoate (SPDB) and N-succinimydyl-4-(2-pyridyldithio)-2-sulfo butanoate (sulfo-SPDB). Disulfide-containing linkers may optionally include additional groups to provide steric hindrance adjacent to the disulfide bond in order to improve the extracellular stability of the linker, for example, inclusion of a geminal dimethyl group. Other cleavable linkers include linkers hydrolyzable at a specific pH or within a pH range, such as hydrazone linkers. Linkers comprising combinations of these functionalities may also be useful, for example, linkers comprising both a hydrazone and a disulfide are known in the art.

A further example of a cleavable linker is a linker comprising a β-glucuronide, which is cleavable by β-glucuronidase, an enzyme present in lysosomes and tumor interstitium (see, for example, De Graaf et al., 2002, Curr. Pharm. Des. 8:1391-1403, and International Patent Publication No. WO 2007/011968). β-glucuronide may also function to improve the hydrophilicity of linker, L.

Another example of a linker that is cleaved internally within a cell and improves hydrophilicity is a linker comprising a pyrophosphate diester moiety (see, for example, Kern et al., 2016, J Am Chem Soc., 138:2430-1445).

In certain embodiments, the linker, L, comprised by the conjugate of Formula (X) is a cleavable linker. In some embodiments, linker, L, comprises a cleavage recognition sequence. In some embodiments, linker, L, may comprise an amino acid sequence that is recognized and cleaved by a lysosomal protease.

Cleavable linkers may optionally further comprise one or more additional functionalities such as self-immolative and self-elimination groups, stretchers or hydrophilic moieties.

Self-immolative and self-elimination groups that find use in linkers include, for example, α-aminobenzyl (PAB) and p-aminobenzyloxycarbonyl (PABC) groups, methylated ethylene diamine (MED) and hemi-aminal groups. Other examples of self-immolative groups include, but are not limited to, aromatic compounds that are electronically similar to the PAB or PABC group such as heterocyclic derivatives, for example 2-aminoimidazol-5-methanol derivatives as described in U.S. Pat. No. 7,375,078. Other examples include groups that undergo cyclization upon amide bond hydrolysis, such as substituted and unsubstituted 4-aminobutyric acid amides (Rodrigues et al., 1995, Chemistry Biology 2:223-227) and 2-aminophenylpropionic acid amides (Amsberry, et al., 1990, J. Org. Chem. 55:5867-5877). Self-immolative/self-elimination groups are typically attached to an amino or hydroxyl group on the compound, D. Self-immolative/self-elimination groups, alone or in combination are often included in peptide-based linkers, but may also be included in other types of linkers.

Stretchers that find use in linkers for drug conjugates include, for example, alkylene groups and stretchers based on aliphatic acids, diacids, amines or diamines, such as diglycolate, malonate, caproate and caproamide. Other stretchers include, for example, glycine-based stretchers and polyethylene glycol (PEG) or monomethoxy polyethylene glycol (mPEG) stretchers.

PEG and mPEG stretchers can also function as hydrophilic moieties within a linker. For example, PEG or mPEG may be included in a linker either “in-line” or as pendant groups to increase the hydrophilicity of the linker (see, for example, U.S.patentt Application Publication No. US 2016/0310612). Various PEG-containing linkers are commercially available from companies such as Quanta BioDesign, Ltd (Plain City, OH). Other hydrophilic groups that may optionally be incorporated into linker, L, include, for example, β-glucuronide, sulfonate groups, carboxylate groups and pyrophosphate diesters.

In certain embodiments, ADCs of Formula (X) may comprise a cleavable linker. In some embodiments, ADCs of Formula (X) may comprise a peptide-containing linker. In some embodiments, ADCs of Formula (X) may comprise a protease-cleavable linker.

In some embodiments, in ADCs of Formula (X), m is 1, and linker, L, is a cleavable linker having Formula (XI):

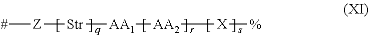

wherein:
Z is a functional group capable of reacting with a target group on the anti-GPC3 antibody construct, T;
Str is a stretcher;
AA₁ and AA₂ are each independently an amino acid, wherein AA₁-[AA₂]r forms a protease cleavage site;
X is a self-immolative group;
q is 0 or 1;
r is 1, 2 or 3;
s is 0, 1 or 2;
#is the point of attachment to the anti-GPC3 antibody construct, T, and
% is the point of attachment to the camptothecin analogue, D.

In some embodiments, in linkers of Formula (XI), q is 1.

In some embodiments, in linkers of Formula (XI), s is 1. In some embodiments, in ADCs of Formula (XI), s is 0.

In some embodiments, in linkers of Formula (XI), r is 1. In some embodiments, in ADCs of Formula (XI), r is 3.

In some embodiments, in linkers of Formula (XI):
Z is

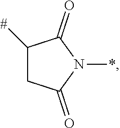

where #is the point of attachment to T, and * is the point of attachment to the remainder of the linker.

In some embodiments, in linkers of Formula (XI), Str is selected from:

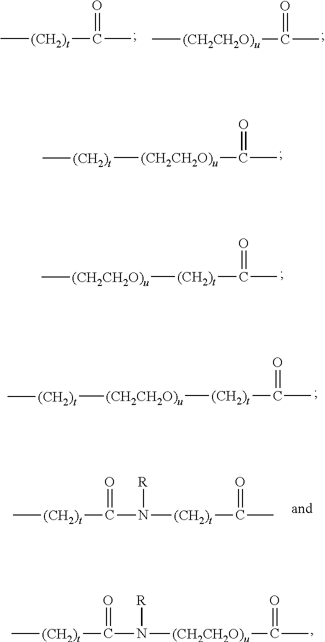

wherein:
R is H or C₁-C₆ alkyl;
t is an integer between 2 and 10, and
u is an integer between 1 and 10.

In some embodiments, in linkers of Formula (XI), Str is selected from:

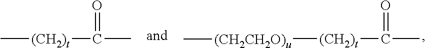

wherein:
t is an integer between 2 and 10, and
u is an integer between 1 and 10.

In some embodiments, in linkers of Formula (XI), AA₁-[AA₂]ᵣ is a dipeptide (i.e. r=1). In some embodiments, in linkers of Formula (XI), AA₁-[AA₂]ᵣ has a sequence selected from: Ala-(D)Asp, Ala-Lys, Ala-Phe, Asn-Lys, Asn-(D)Lys, Asp-Val, His-Val, Ile-Cit, Ile-Pro, Ile-Val, Leu-Cit, Me3Lys-Pro, Met-Lys, Met-(D)Lys, NorVal-(D)Asp, Phe-Arg, Phe-Cit, Phe-Lys, PhenylGly-(D)Lys, Pro-(D)Lys, Trp-Cit, Val-Ala, Val-(D)Asp, Val-Cit, Val-Gly, Val-Gln and Val-Lys.

In some embodiments, in linkers of Formula (XI), AA₁-[AA₂]ᵣ is a tripeptide (i.e. r=2). In some embodiments, in linkers of Formula (XI), AA₁-[AA₂]ᵣ has a sequence selected from: Ala-Ala-Asn, Ala-Val-Cit, (D)Ala-Phe-Lys, Asp-Val-Ala, Asp-Val-Cit, Gly-Cit-Val, Lys-Val-Ala, Lys-Val-Cit, Met-Cit-Val, (D)Phe-Phe-Lys, and Asn-Pro-Val.

In some embodiments, in linkers of Formula (XI), AA₁-[AA₂]ᵣ is a tetrapeptide (i.e. r=3). In some embodiments, in linkers of Formula (XI), AA₁-[AA₂]ᵣ has a sequence selected from: Ala-Leu-Ala-Leu, Gly-Phe-Leu-Gly, Gly-Gly-Phe-Gly and Gly-Phe-Gly-Gly.

In certain embodiments, in ADCs of Formula (X), m is 1, and linker, L, is a cleavable linker having Formula (XII):

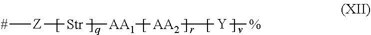

wherein:
Z is a functional group capable of reacting with a target group on the anti-GPC3 antibody
construct, T;
Str is a stretcher;
AA₁ and AA₂ are each independently an amino acid, wherein AA₁-[AA₂]ᵣ forms a protease
cleavage site;
Y is —NH—CH₂—;
q is 0 or 1;
r is 1, 2 or 3;
v is 0 or 1;
#is the point of attachment to the anti-GPC3 antibody construct, T, and
% is the point of attachment to the camptothecin analogue, D.

In some embodiments, in linkers of Formula (XII), q is 1.

In some embodiments, in linkers of Formula (XII), v is 0. In some embodiments, in ADCs of Formula (XII), s is 1.

In some embodiments, in linkers of Formula (XII), r is 1. In some embodiments, in ADCs of Formula (XII), r is 3.

In some embodiments, in linkers of Formula (XII):
Z is

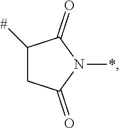

where #is the point of attachment to T, and * is the point of attachment to the remainder of the linker.

In some embodiments, in linkers of Formula (XII), Str is selected from:

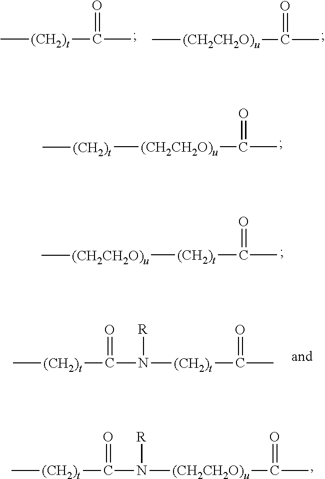

wherein:
R is H or C₁-C₆ alkyl;
t is an integer between 2 and 10, and
u is an integer between 1 and 10.

In some embodiments, in linkers of Formula (XII), Str is selected from:

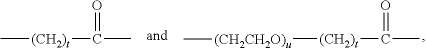

wherein:
t is an integer between 2 and 10, and
u is an integer between 1 and 10.

In some embodiments, in linkers of Formula (XII), AA₁-[AA₂]ᵣ is a dipeptide (i.e. r=1).

In some embodiments, in linkers of Formula (XII), AA₁-[AA₂]ᵣ has a sequence selected from: Ala-(D)Asp, Ala-Lys, Ala-Phe, Asn-Lys, Asn-(D)Lys, Asp-Val, His-Val, Ile-Cit, Ile-Pro, Ile-Val, Leu-Cit, Me3Lys-Pro, Met-Lys, Met-(D)Lys, NorVal-(D)Asp, Phe-Arg, Phe-Cit, Phe-Lys, PhenylGly-(D)Lys, Pro-(D)Lys, Trp-Cit, Val-Ala, Val-(D)Asp, Val-Cit, Val-Gly, Val-Gln and Val-Lys.

In some embodiments, in linkers of Formula (XII), AA₁-[AA₂]ᵣ is a tripeptide (i.e. r=2).

In some embodiments, in linkers of Formula (XII), AA₁-[AA₂]ᵣ has a sequence selected from: Ala-Ala-Asn, Ala-Val-Cit, (D)Ala-Phe-Lys, Asp-Val-Ala, Asp-Val-Cit, Gly-Cit-Val, Lys-Val-Ala, Lys-Val-Cit, Met-Cit-Val, (D)Phe-Phe-Lys, Asn-Pro-Val.

In some embodiments, in linkers of Formula (XII), AA₁-[AA₂]ᵣ is a tetrapeptide (i.e. r=3). In some embodiments, in linkers of Formula (XII), AA₁-[AA₂]ᵣ has a sequence selected from: Ala-Leu-Ala-Leu, Gly-Phe-Leu-Gly, Gly-Gly-Phe-Gly and Gly-Phe-Gly-Gly.

In some embodiments, in linkers of Formula (XII), Y is —NH—CH₂. In some embodiments, in linkers of Formula (XII), v is 1 and Y is —NH—CH₂.

In some embodiments, ADCs of Formula (X) may comprise a disulfide-containing linker.

In some embodiments, in ADCs of Formula (X), m is 1, and linker, L, is a cleavable linker having Formula (XIII):

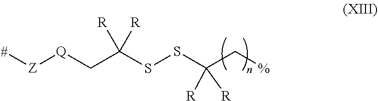

wherein:
Z is a functional group capable of reacting with a target group on the anti-GPC3 antibody construct, T;
Q is —(CH₂)ₚ— or —(CH₂CH₂O)_{q}—, wherein p and q are each independently an integer between 1 and 10;
each R is independently H or C₁-C₆ alkyl;
n is 1, 2 or 3;
#is the point of attachment to the anti-GPC3 antibody construct, T, and
% is the point of attachment to the camptothecin analogue, D.

In some embodiments, ADCs of Formula (X) may comprise a P-glucuronide-containing linker.

Various non-cleavable linkers are known in the art for linking drugs to targeting moieties and may be useful in the ADCs of the present disclosure in certain embodiments. Examples of non-cleavable linkers include linkers having an N-succinimidyl ester or N-sulfosuccinimidyl ester moiety for reaction with the anti-GPC3 antibody construct, as well as a maleimido- or haloacetyl-based moiety for reaction with the camptothecin analogue, or vice versa. An example of such a non-cleavable linker is based on sulfosuccinimidyl-4-[N-maleimidomethyl]cyclohexane-1-carboxylate (sulfo-SMCC). Sulfo-SMCC conjugation typically occurs via a maleimide group which reacts with sulfhydryls (thiols, SH) on the camptothecin analogue, while the sulfo-NHS ester is reactive toward primary amines (as found in lysine and at the N-terminus of proteins or peptides) on the anti-GPC3 antibody construct. Other non-limiting examples of such linkers include those based on N-succinimidyl 4-(maleimidomethyl)cyclohexanecarboxylate (SMCC), N-succinimidyl-4-(N-maleimidomethyl)-cyclohexane-1-carboxy-(6-amidocaproate) (“long chain” SMCC or LC-SMCC), x-maleimidoundecanoic acid N-succinimidyl ester (KMUA), β-maleimidobutyric acid N-succinimidyl ester (GMBS), 8-maleimidocaproic acid N-hydroxysuccinimide ester (EMCS), m-maleimidobenzoyl-N-hydroxysuccinimide ester (MBS), N-(α-maleimidoacetoxy)-succinimide ester (AMAS), succinimidyl-6-(β-maleimidopropionamido)hexanoate (SMPH), N-succinimidyl 4-(γ-maleimidophenyl)-butyrate (SMPB) and N-(p-maleimidophenyl)isocyanate (PMPI). Other examples include those comprising a haloacetyl-based functional group such as N-succinimidyl-4-(iodoacetyl)-aminobenzoate (SIAB), N-succinimidyl iodoacetate (SIA), N-succinimidyl bromoacetate (SBA) and N-succinimidyl 3-(bromoacetamido)propionate (SBAP).

Non-limiting examples of drug-linkers comprising camptothecin analogues of Formula (I) are shown in Table 7, Table 8, and Table 9. Non-limiting examples of conjugates comprising these drug-linkers are shown in Table 10, Table 11 and Table 12. In certain embodiments, the ADC of Formula (X) comprises a drug-linker selected from the drug-linkers shown in Tables 7, 8 and 9. In certain embodiments, the ADC of Formula (X) is selected from the conjugates shown in Tables 10, 11 and 12, where T is the anti-GPC3 antibody construct and n is between 1 and 10. In some embodiments, the ADC of Formula (X) is selected from the conjugates shown in Tables 10, 11 and 12, where T is the anti-GPC3 antibody construct and n is between 2 and 8. In some embodiments, the ADC of Formula (X) is selected from the conjugates shown in Tables 10, 11, and 12, where T is the anti-FRa antibody construct and n is between 4 and 8.

In certain embodiments, the ADC of Formula (X) comprises a drug-linker (L-(D)ₘ) selected from MT-GGFG-AM-Compound 139, MC-GGFG-AM-Compound 139, MT-GGFG-Compound 140, MC-GGFG-Compound 140, MT-GGFG-AM-Compound 141, MC-GGFG-AM-Compound 141, MT-GGFG-Compound 141, MC-GGFG-Compound 141, MT-GGFG-Compound 148 and MC-GGFG-Compound 148, and n is 4 or 8. In some embodiments, the ADC of Formula (X) comprises a drug-linker (L-(D)ₘ) selected from MT-GGFG-AM-Compound 139, MC-GGFG-AM-Compound 139, MT-GGFG-Compound 140, MC-GGFG-Compound 140, MT-GGFG-AM-Compound 141, MC-GGFG-AM-Compound 141, MT-GGFG-Compound 141, MC-GGFG-Compound 141, MT-GGFG-Compound 148 and MC-GGFG-Compound 148, and n is 8.

### Preparation of ADCs

ADCs of Formula (X) may be prepared by standard methods known in the art (see, for example, Bioconjugate Techniques (G. T. Hermanson, 2013, Academic Press)). Various linkers and linker components are commercially available or may be prepared using standard synthetic organic chemistry techniques (see, for example, March's Advanced Organic Chemistry (Smith & March, 2006, Sixth Ed., Wiley); Toki et al., (2002) J. Org. Chem. 67:1866-1872; Frisch et al., (1997) Bioconj. Chem. 7:180-186; Bioconjugate Techniques (G. T. Hermanson, 2013, Academic Press)). In addition, various antibody drug conjugation services are available commercially from companies such as Lonza Inc. (Allendale, NJ), Abzena PLC (Cambridge, UK), ADC Biotechnology (St. Asaph, UK), Baxter BioPharma Solutions (Baxter Healthcare Corporation, Deerfield, IL) and Piramal Pharma Solutions (Grangemouth, UK).

Typically, preparation of the ADCs comprises first preparing a drug-linker, D-L, comprising one or more camptothecin analogues of Formula (I) and linker L, and then conjugating the drug-linker, D-L, to an appropriate group on the anti-GPC3 antibody construct, T. Ligation of linker, L, to the anti-GPC3 antibody construct, T, and subsequent ligation of the anti-GPC3 antibody construct-linker, T-L, to one or more camptothecin analogues of Formula (I), D, remains however an alternative approach that may be employed in some embodiments.

Suitable groups on compounds of Formula (I), D, for attachment of linker, L, in either of the above approaches include, but are not limited to, thiol groups, amine groups, carboxylic acid groups and hydroxyl groups. In some embodiments of the present disclosure, linker, L, is attached to a compound of Formula (I), D, via a hydroxyl or amine group on the compound.

Suitable groups on the anti-GPC3 antibody construct, T, for attachment of linker, L, in either of the above approaches include sulfhydryl groups (for example, on the side-chain of cysteine residues), amino groups (for example, on the side-chain of lysine residues), carboxylic acid groups (for example, on the side-chains of aspartate or glutamate residues), and carbohydrate groups.

For example, the anti-GPC3 antibody construct T may comprise one or more naturally occurring sulfhydryl groups allowing the anti-GPC3 antibody construct, T, to bond to linker, L, via the sulfur atom of a sulfhydryl group. Alternatively, the anti-GPC3 antibody construct, T, may comprise one or more lysine residues that can be chemically modified to introduce one or more sulfhydryl groups. Reagents that can be used to modify lysine residues include, but are not limited to, N-succinimidyl S-acetylthioacetate (SATA), N-succinimidyl-3-(2-pyridyldithio)propionate (“SPDP”) and 2-iminothiolane hydrochloride (Traut's Reagent). Alternatively, the anti-GPC3 antibody construct, T, may comprise one or more carbohydrate groups that can be chemically modified to include one or more sulfhydryl groups.

Carbohydrate groups on the anti-GPC3 antibody construct, T, may also be oxidized to provide an aldehyde (—CHO) group (see, for example, Laguzza et al., 1989, J. Med. Chem. 32(3):548-55), which could subsequently be reacted with linker, L, for example, via a hydrazine or hydroxylamine group on linker, L.

The anti-GPC3 antibody construct, T, may also be modified to include additional cysteine residues (see, for example, U.S. Pat. Nos. 7,521,541; 8,455,622 and 9,000,130) or non-natural amino acids that provide reactive handles, such as selenomethionine, p-acetylphenylalanine, formylglycine or p-azidomethyl-L-phenylalanine (see, for example, Hofer et al., 2009, Biochemistry, 48:12047-12057; Axup et al., 2012, PNAS, 109:16101-16106; Wu et al., 2009, PNAS, 106:3000-3005; Zimmerman et al., 2014, Bioconj. Chem., 25:351-361), to allow for site-specific conjugation. Alternatively, the anti-GPC3 antibody construct, T, may be modified to include a non-natural reactive group, such as an azide, that allows for conjugation to the linker via a complementary reactive group on the linker, for example, for example, by click chemistry (see, for example, Chio & Bane, 2020, Methods Mol. Biol., 2078:83-97). A further option is the use of GlycoConnect™ technology (Synaffix B V, Nijmegen, Netherlands), which involves enzymatic remodelling of the antibody glycans to allow for attachment of a linker by metal-free click chemistry (see, for example, European Patent No. EP 2 911 699).

Other protocols for the modification of proteins for the attachment or association of linker, L, are known in the art and include those described in Coligan et al., Current Protocols in Protein Science, vol. 2, John Wiley & Sons (2002).

Alternatively, ADCs may be prepared using the enzyme transglutaminase, in particular, bacterial transglutaminase (BTG) from Streptomyces mobaraensis (see, for example, Jeger et al., 2010, Angew. Chem. Int. Ed., 49:9995-9997). BTG forms an amide bond between the side chain carboxamide of a glutamine (the amine acceptor, typically on the antibody) and an alkyleneamino group (the amine donor, typically on the drug-linker), which can be, for example, the F-amino group of a lysine or a 5-amino-n-pentyl group. Antibodies may also be modified to include a glutamine containing peptide, or “tag,” which allows BTG conjugation to be used to conjugate the antibody to a drug-linker (see, for example, U.S. Patent Application Publication No. US 2013/0230543 and International (PCT) Publication No. WO 2016/144608).

A similar conjugation approach utilizes the enzyme sortase A. In this approach, the antibody is typically modified to include the sortase A recognition motif (LPXTG, where X is any natural amino acid) and the drug-linker is designed to include an oligoglycine motif (typically GGG) to allow for sortase A-mediated transpeptidation (see, for example, Beerli, et al., 2015, PLos One, 10:e0131177; Chen et al., 2016, Nature:Scientific Reports, 6:31899).

Once conjugation is complete, the average number of compounds of Formula (I) conjugated to the anti-GPC3 antibody construct, T, (i.e. the “drug-to-antibody ratio” or DAR) may be determined by standard techniques such as UV/VIS spectroscopic analysis, ELISA-based techniques, chromatography techniques such as hydrophobic interaction chromatography (HIC), UV-MALDI mass spectrometry (MS) and MALDI-TOF MS. In addition, distribution of drug-linked forms (for example, the fraction of the anti-GPC3 antibody construct, T, containing zero, one, two, three, etc. compounds of Formula (I), D) may also optionally be analyzed. Various techniques are known in the art to measure DAR distribution, including MS (with or without an accompanying chromatographic separation step), hydrophobic interaction chromatography, reverse-phase HPLC or iso-electric focusing gel electrophoresis (IEF) (see, for example, Wakankar et al., 2011, mAbs, 3:161-172).

### PHARMACEUTICAL COMPOSITIONS

For therapeutic uses, the ADCs of the present disclosure are typically formulated as pharmaceutical compositions. Certain embodiments of the present disclosure thus relate to pharmaceutical compositions comprising an ADC as described herein and a pharmaceutically acceptable carrier, diluent, or excipient. Such pharmaceutical compositions may be prepared by known procedures using well-known and readily available ingredients.

Pharmaceutical compositions may be formulated for administration to a subject by, for example, oral (including, for example, buccal or sublingual), topical, parenteral, rectal or vaginal routes, or by inhalation or spray. The term “parenteral” as used herein includes subcutaneous injection, and intradermal, intra-articular, intravenous, intramuscular, intravascular, intrasternal, intrathecal injection or infusion. The pharmaceutical composition will typically be formulated in a format suitable for administration to the subject, for example, as a syrup, elixir, tablet, troche, lozenge, hard or soft capsule, pill, suppository, oily or aqueous suspension, dispersible powder or granule, emulsion, injectable or solution. Pharmaceutical compositions may be provided as unit dosage formulations.

In certain embodiments, the pharmaceutical compositions comprising the ADCs are formulated for parenteral administration, for example as lyophilized formulations or aqueous solutions. Such pharmaceutical compositions may be provided, for example, in a unit dosage injectable form.

Pharmaceutically acceptable carriers are generally nontoxic to recipients at the dosages and concentrations employed. Examples of such carriers include, but are not limited to, buffers such as phosphate, citrate, and other organic acids; antioxidants such as ascorbic acid and methionine; preservatives such as octadecyldimethylbenzyl ammonium chloride, hexamethonium chloride, benzalkonium chloride, benzethonium chloride, phenol, butyl alcohol, benzyl alcohol, alkyl parabens (such as methyl or propyl paraben), catechol, resorcinol, cyclohexanol, 3-pentanol and m-cresol; low molecular weight (less than about 10 residues) polypeptides; proteins such as serum albumin or gelatin; hydrophilic polymers such as polyvinylpyrrolidone; amino acids such as glycine, glutamine, asparagine, histidine, arginine or lysine; monosaccharides, disaccharides, and other carbohydrates such as glucose, mannose or dextrins; chelating agents such as EDTA; sugars such as sucrose, mannitol, trehalose or sorbitol; salt-forming counter-ions such as sodium; metal complexes such as Zn-protein complexes, and non-ionic surfactants such as polyethylene glycol (PEG).

In certain embodiments, the compositions comprising the ADCs may be in the form of a sterile injectable aqueous or oleaginous solution or suspension. Such suspensions may be formulated using suitable dispersing or wetting agents and/or suspending agent that are known in the art. The sterile injectable solution or suspension may comprise the ADC in a non-toxic parentally acceptable diluent or carrier. Acceptable diluents and carriers that may be employed include, for example, 1,3-butanediol, water, Ringer's solution or isotonic sodium chloride solution. In addition, sterile, fixed oils may be employed as a carrier. For this purpose, various bland fixed oils may be employed, including synthetic mono- or diglycerides. In addition, fatty acids such as oleic acid find use in the preparation of injectables. Adjuvants such as local anaesthetics, preservatives and/or buffering agents may also be included in the injectable solution or suspension.

In certain embodiments, the composition comprising the ADC may be formulated for intravenous administration to humans. Typically, compositions for intravenous administration are solutions in sterile isotonic aqueous buffer. Where necessary, the composition may also include a solubilizing agent and/or a local anaesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, PA (2000).

### METHODS OF USE

Certain embodiments of the present disclosure relate to the therapeutic use of the ADCs described herein. Some embodiments relate to the use of the ADCs as therapeutic agents.

Certain embodiments of the present disclosure relate to methods of inhibiting abnormal cancer cell or tumor cell growth; inhibiting cancer cell or tumor cell proliferation, or treating cancer in a subject, comprising administering an ADC described herein. In certain embodiments, the ADCs described herein may be used in the treatment of cancer. Some embodiments of the present disclosure thus relate to the use of the ADCs as anti-cancer agents.

Certain embodiments of the present disclosure relate to methods of inhibiting the proliferation of cancer or tumor cells comprising contacting the cells with an ADC as described herein, for example, an ADC of Formula (X). Some embodiments relate to a method of killing cancer or tumor cells comprising contacting the cells with an ADC as described herein, for example, an ADC of Formula (X).

Some embodiments relate to methods of treating a subject having a cancer by administering to the subject an ADC as described herein, for example, an ADC of Formula (X). In this context, treating the subject may result in one or more of a reduction in the size of a tumor, the slowing or prevention of an increase in the size of a tumor, an increase in the disease-free survival time between the disappearance or removal of a tumor and its reappearance, prevention of a subsequent occurrence of a tumor (for example, metastasis), an increase in the time to progression, reduction of one or more adverse symptom associated with a tumor, and/or an increase in the overall survival time of a subject having cancer.

Certain embodiments relate to the use of an ADC as described herein, for example, an ADC of Formula (X), in a method of inhibiting tumor growth in a subject. Some embodiments relate to the use of an ADC as described herein, for example, an ADC of Formula (X), in a method of inhibiting proliferation of and/or killing cancer cells in vitro. Some embodiments relate to the use of an ADC as described herein, for example, an ADC of Formula (X), in a method of inhibiting proliferation of and/or killing cancer cells in vivo in a subject having a cancer.

Examples of cancers which may be treated in certain embodiments are carcinomas, including adenocarcinomas and squamous cell carcinomas; melanomas and sarcomas. Carcinomas and sarcomas are also frequently referred to as “solid tumors.” Examples of commonly occurring solid tumors that may be treated in certain embodiments include, but are not limited to, brain cancer, breast cancer, cervical cancer, colon cancer, head and neck cancer, kidney cancer, liver cancer, lung cancer, ovarian cancer, pancreatic cancer, prostate cancer, stomach cancer, uterine cancer, non-small cell lung cancer (NSCLC) and colorectal cancer. Various forms of lymphoma also may result in the formation of a solid tumor and, therefore, may also be considered to be solid tumors in certain situations. Typically, the cancer to be treated is a GPC3-expressing cancer.

Certain embodiments relate to methods of inhibiting the growth of GPC3-positive tumor cells comprising contacting the cells with an ADC as described herein, for example, an ADC of Formula (X). The cells may be in vitro or in vivo. In certain embodiments, the ADCs may be used in methods of treating a GPC3-positive cancer or tumor in a subject.

In some embodiments, the ADCs described herein may be used to treat subject having a cancer that overexpresses GPC3. Cancers that overexpress GPC3 are typically solid tumors.

Examples include, but are not limited to, hepatocellular carcinoma (HCC), melanoma, lung carcinoma, and hepatoblastoma.

### PHARMACEUTICAL KITS

Certain embodiments relate to pharmaceutical kits comprising an ADC as described herein, for example, an ADC of Formula (X).

The kit typically will comprise a container holding the ADC and a label and/or package insert on or associated with the container. The label or package insert contains instructions customarily included in commercial packages of therapeutic products, providing information about the indications, usage, dosage, administration, contraindications and/or warnings concerning the use of such therapeutic products. The label or package insert may further include a notice in the form prescribed by a governmental agency regulating the manufacture, use or sale of pharmaceuticals or biological products, which notice reflects approval by the agency of manufacture, for use or sale for human or animal administration. In some embodiments, the container may have a sterile access port. For example, the container may be an intravenous solution bag or a vial having a stopper that may be pierced by a hypodermic injection needle.

In addition to the container holding the ADC, the kit may optionally comprise one or more additional containers comprising other components of the kit. For example, a pharmaceutically acceptable buffer (such as bacteriostatic water for injection (BWFI), phosphate-buffered saline, Ringer's solution or dextrose solution), other buffers or diluents.

Suitable containers include, for example, bottles, vials, syringes, intravenous solution bags, and the like. The containers may be formed from a variety of materials such as glass or plastic. If appropriate, one or more components of the kit may be lyophilized or provided in a dry form, such as a powder or granules, and the kit can additionally contain a suitable solvent for reconstitution of the lyophilized or dried component(s).

The kit may further include other materials desirable from a commercial or user standpoint, such as filters, needles, and syringes.

### Tables 7 to 12

TABLE 7
Exemplary drug-linker (DL) structures comprising camptothecin analogues of
Formula (I) with a C7 linkage

| Name | Structure |
|---|---|
| DL: MT- GGFG- Compound 104 | 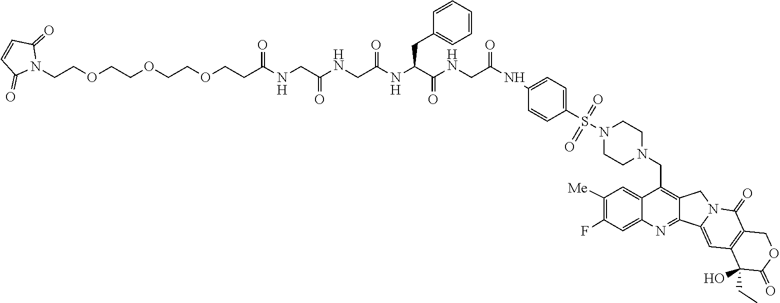 [SMILES: CC[C@@]1(O)C(=O)OCc2c1cc1n(c2=O)Cc2c-1nc1cc(F)c(C)cc1c2CN1CCN(S(=O)(=O)c2ccc(NC(=O)CNC(=O)[C@H](Cc3ccccc3)NC(=O)CNC(=O)CNC(=O)CCOCCOCCOCCN3C(=O)C=CC3=O)cc2)CC1] |
| DL: MT- GGFG- Compound 108 | 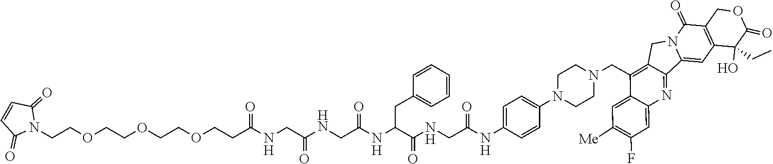 [SMILES: CC[C@@]1(O)C(=O)OCc2c1cc1n(c2=O)Cc2c-1nc1cc(F)c(C)cc1c2CN1CCN(c2ccc(NC(=O)CNC(=O)C(Cc3ccccc3)NC(=O)CNC(=O)CNC(=O)CCOCCOCCOCCN3C(=O)C=CC3=O)cc2)CC1] |
| DL: MT- GGFG- Compound 127 | 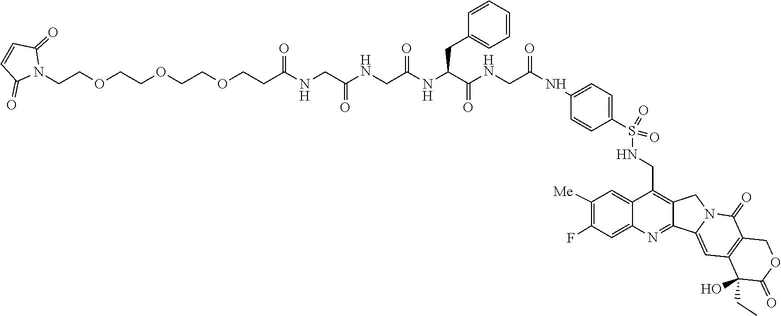 [SMILES: CC[C@@]1(O)C(=O)OCc2c1cc1n(c2=O)Cc2c-1nc1cc(F)c(C)cc1c2CNS(=O)(=O)c1ccc(NC(=O)CNC(=O)[C@H](Cc2ccccc2)NC(=O)CNC(=O)CNC(=O)CCOCCOCCOCCN2C(=O)C=CC2=O)cc1] |
| DL: MT- GGFG- AM- Compound 136 | 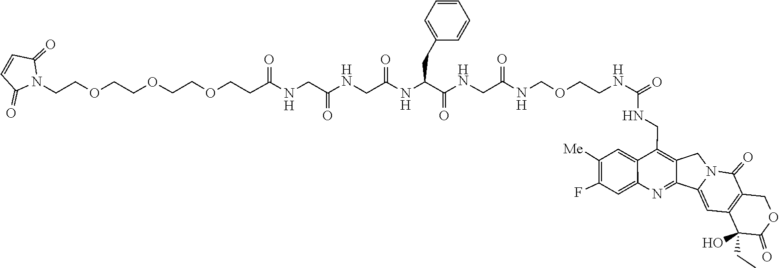 [SMILES: CC[C@@]1(O)C(=O)OCc2c1cc1n(c2=O)Cc2c-1nc1cc(F)c(C)cc1c2CNC(=O)NCCOCNC(=O)CNC(=O)[C@H](Cc1ccccc1)NC(=O)CNC(=O)CNC(=O)CCOCCOCCOCCN1C(=O)C=CC1=O] |
| DL: MT- GGFG- AM- Compound 139 | 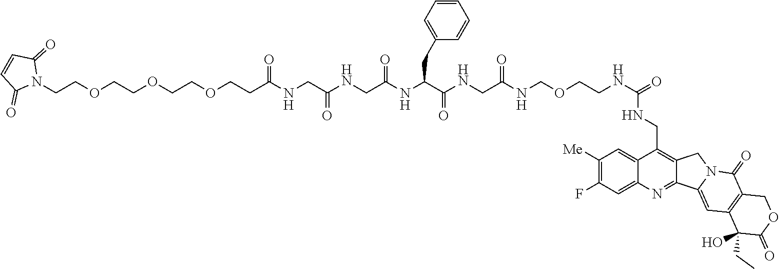 [SMILES: CC[C@@]1(O)C(=O)OCc2c1cc1n(c2=O)Cc2c-1nc1cc(F)c(C)cc1c2CNC(=O)NCCOCNC(=O)CNC(=O)[C@H](Cc1ccccc1)NC(=O)CNC(=O)CNC(=O)CCOCCOCCOCCN1C(=O)C=CC1=O] |
| DL: MT- GGFG- AM- Compound 129 | 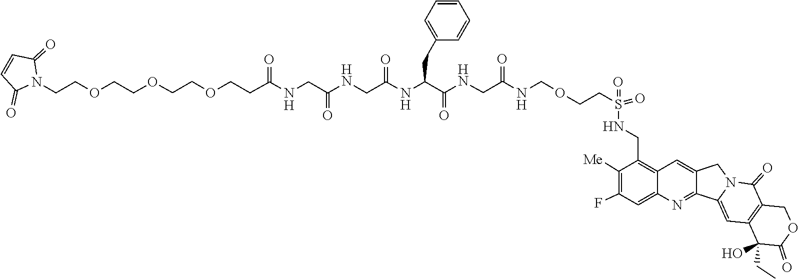 [SMILES: CC[C@@]1(O)C(=O)OCc2c1cc1n(c2=O)Cc2cc3c(CNS(=O)(=O)CCOCNC(=O)CNC(=O)[C@H](Cc4ccccc4)NC(=O)CNC(=O)CNC(=O)CCOCCOCCOCCN4C(=O)C=CC4=O)c(C)c(F)cc3nc2-1] |
| DL: MT- GGFG- AM- Compound 113 | 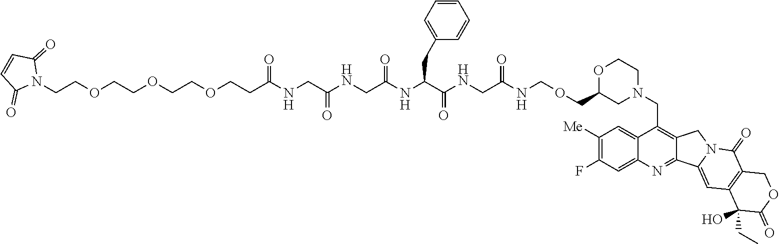 [SMILES: CC[C@@]1(O)C(=O)OCc2c1cc1n(c2=O)Cc2c-1nc1cc(F)c(C)cc1c2CN1CCO[C@H](COCNC(=O)CNC(=O)[C@H](Cc2ccccc2)NC(=O)CNC(=O)CNC(=O)CCOCCOCCOCCN2C(=O)C=CC2=O)C1] |
| DL: MT- GGFG- AM- Compound 141 | 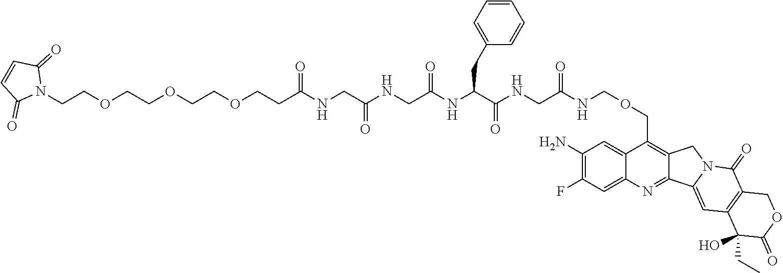 [SMILES: CC[C@@]1(O)C(=O)OCc2c1cc1n(c2=O)Cc2c-1nc1cc(F)c(N)cc1c2COCNC(=O)CNC(=O)[C@H](Cc1ccccc1)NC(=O)CNC(=O)CNC(=O)CCOCCOCCOCCN1C(=O)C=CC1=O] |
| DL: MC- GGFG- AM- Compound 141 | 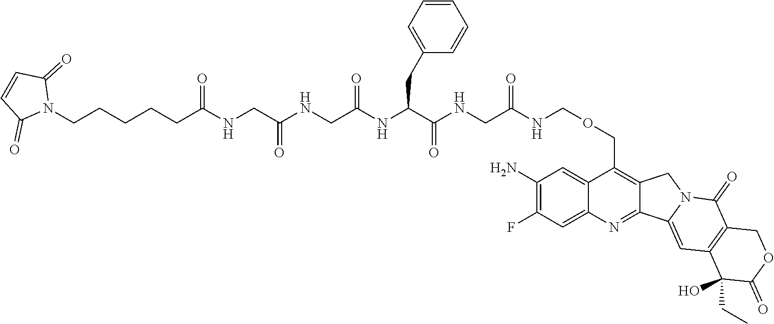 [SMILES: CC[C@@]1(O)C(=O)OCc2c1cc1n(c2=O)Cc2c-1nc1cc(F)c(N)cc1c2COCNC(=O)CNC(=O)[C@H](Cc1ccccc1)NC(=O)CNC(=O)CNC(=O)CCCCCN1C(=O)C=CC1=O] |
| DL: MT- GGFG- AM- Compound 117 | 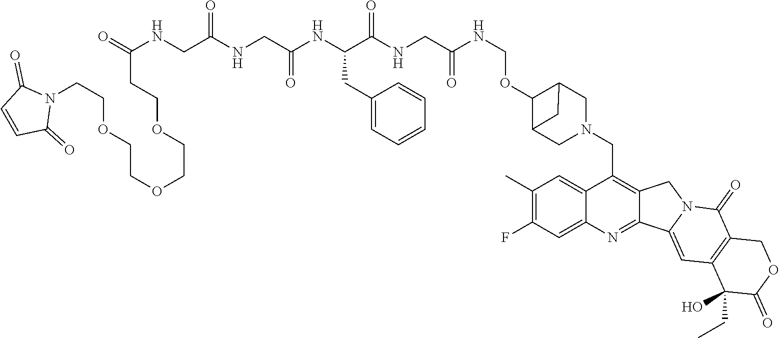 [SMILES: CC[C@@]1(O)C(=O)OCc2c1cc1n(c2=O)Cc2c-1nc1cc(F)c(C)cc1c2CN1CC2CC(C1)C2OCNC(=O)CNC(=O)[C@H](Cc1ccccc1)NC(=O)CNC(=O)CNC(=O)CCOCCOCCOCCN1C(=O)C=CC1=O] |
| DL: MT- GGFG- AM- Compound 118 | 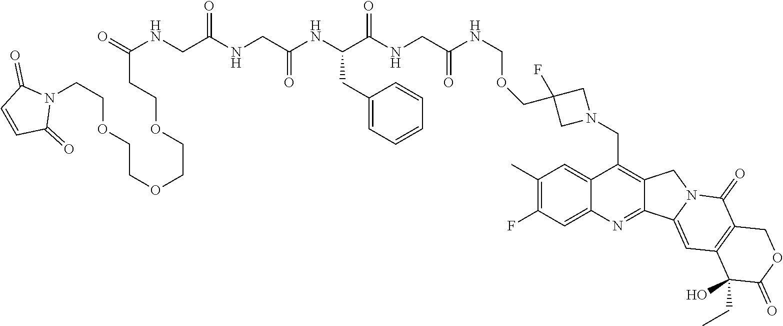 [SMILES: CC[C@@]1(O)C(=O)OCc2c1cc1n(c2=O)Cc2c-1nc1cc(F)c(C)cc1c2CN1CC(F)(COCNC(=O)CNC(=O)[C@H](Cc2ccccc2)NC(=O)CNC(=O)CNC(=O)CCOCCOCCOCCN2C(=O)C=CC2=O)C1] |
| DL: MC- GGFG- AM- Compound 139 | 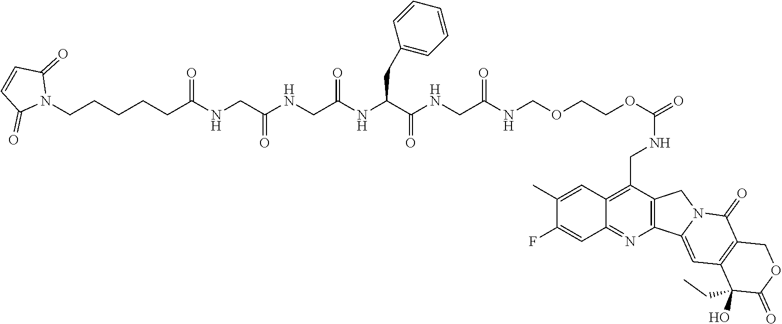 [SMILES: CC[C@@]1(O)C(=O)OCc2c1cc1n(c2=O)Cc2c-1nc1cc(F)c(C)cc1c2CNC(=O)OCCOCNC(=O)CNC(=O)[C@H](Cc1ccccc1)NC(=O)CNC(=O)CNC(=O)CCCCCN1C(=O)C=CC1=O] |
| DL: DIS- Compound 145 | 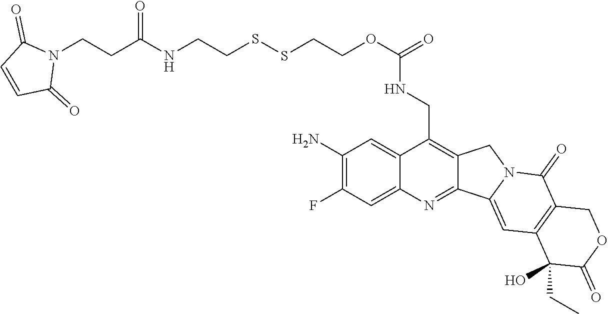 [SMILES: CC[C@@]1(O)C(=O)OCc2c1cc1n(c2=O)Cc2c-1nc1cc(F)c(N)cc1c2CNC(=O)OCCSSCCNC(=O)CCN1C(=O)C=CC1=O] |

TABLE 8
Exemplary drug-linker (DL) structures comprising camptothecin analogues of
Formula (I) with a C10 linkage

| Name | Structure |
|---|---|
| DL: MT- GGFG- Compound 140 | 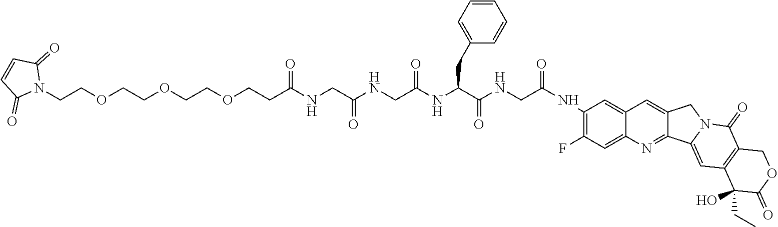 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)CCOCCOCCOCCN4C(=O)C=CC4=O)c(F)cc3nc2-1] |
| DL: MT- GGFG- Compound 141 | 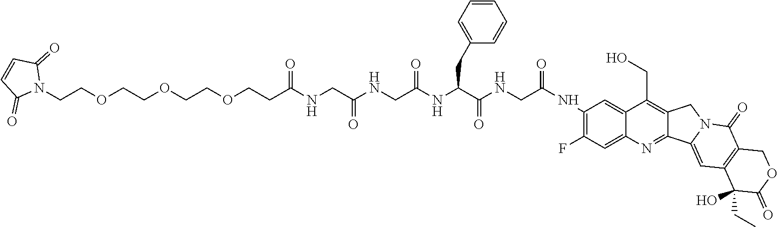 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CO] |
| DL: MT- GGFG- Compound 145 | 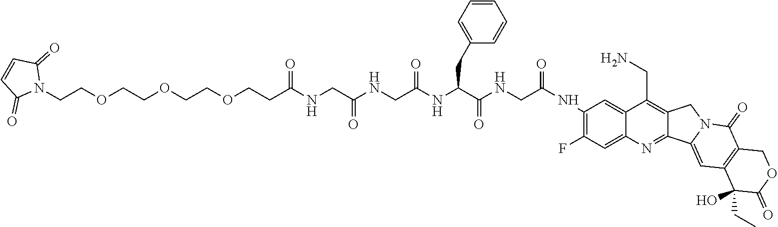 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN] |
| DL: MT- GGFG- Compound 148 | 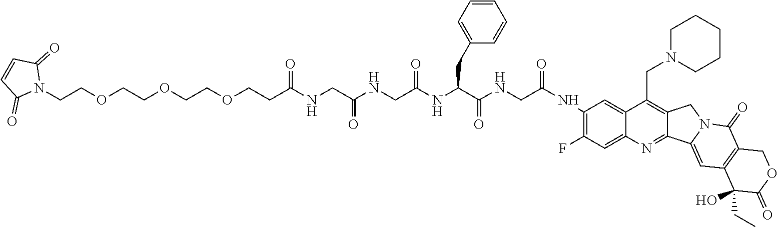 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN1CCCCC1] |
| DL: MC- GGFG- Compound 140 | 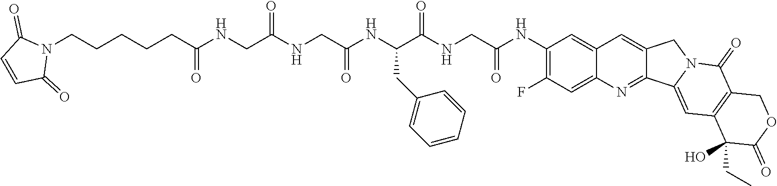 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)CCCCCN4C(=O)C=CC4=O)c(F)cc3nc2-1] |
| DL: MT- GGFG- Compound 142 | 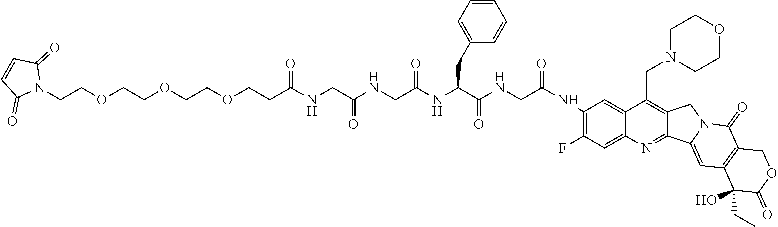 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN1CCOCC1] |
| DL: MC- GGFG- Compound 141 | 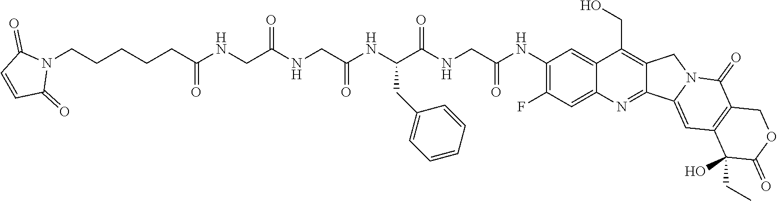 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCCCCN3C(=O)C=CC3=O)cc1c2CO] |
| DL: MC- VA- Compound 140 (DL: 201) | 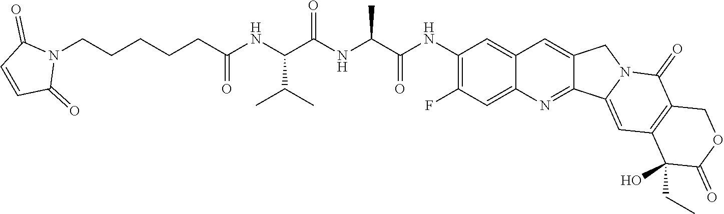 [SMILES: CC[C@@]1(O)C(=O)OCc2c1cc1n(c2=O)Cc2cc3cc(NC(=O)[C@H](C)NC(=O)[C@@H](NC(=O)CCCCCN4C(=O)C=CC4=O)C(C)C)c(F)cc3nc2-1] |
| DL: NHC- C-VA- Compound 140 | 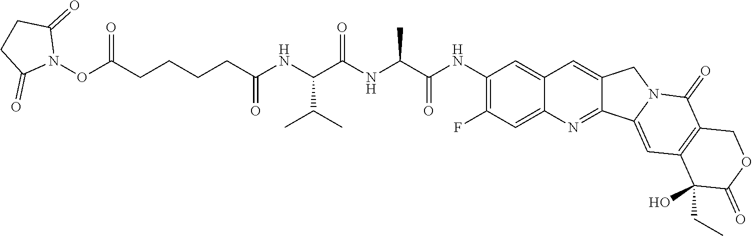 [SMILES: CC[C@@]1(O)C(=O)OCc2c1cc1n(c2=O)Cc2cc3cc(NC(=O)[C@H](C)NC(=O)[C@@H](NC(=O)CCCCC(=O)ON4C(=O)CCC4=O)C(C)C)c(F)cc3nc2-1] |
| DL: Azido- PEG8- VA- Compound 140 | 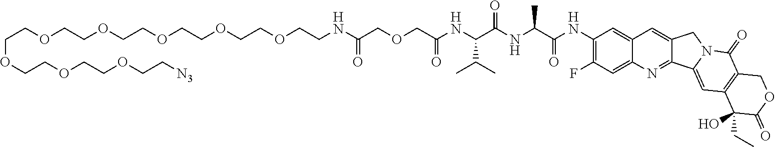 [SMILES: CC[C@@]1(O)C(=O)OCc2c1cc1n(c2=O)Cc2cc3cc(NC(=O)[C@H](C)NC(=O)[C@@H](NC(=O)COCC(=O)NCCOCCOCCOCCOCCOCCOCCOCCOCCN=[N+]=[N-])C(C)C)c(F)cc3nc2-1] |
| DL: MT- GGFG- Compound 140 | 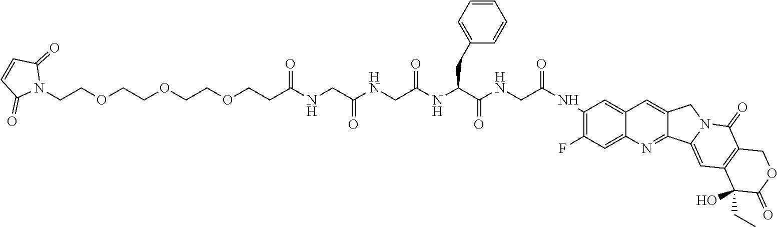 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)CCOCCOCCOCCN4C(=O)C=CC4=O)c(F)cc3nc2-1] |
| DL: MT- GGFG- Compound 141 | 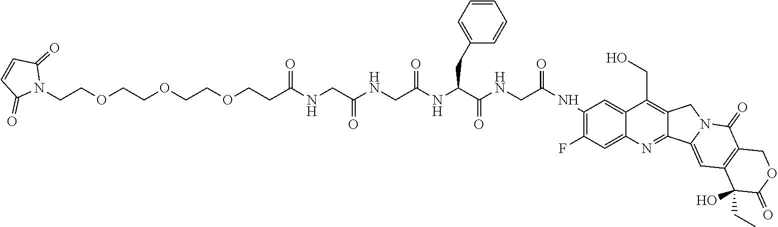 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CO] |
| DL: MT- GGFG- Compound 145 | 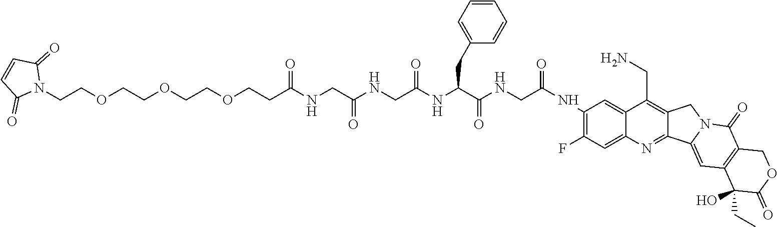 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN] |
| DL: MT- GGFG- Compound 148 | 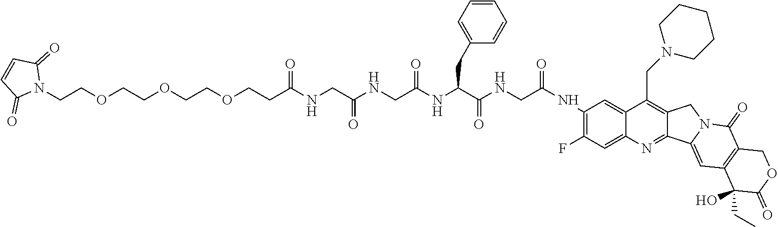 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN1CCCCC1] |
| DL: MC- GGFG- Compound 140 | 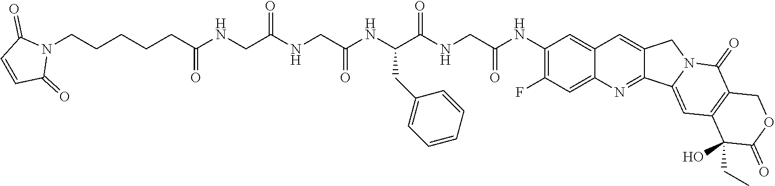 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)CCCCCN4C(=O)C=CC4=O)c(F)cc3nc2-1] |
| DL: MT- GGFG- Compound 142 | 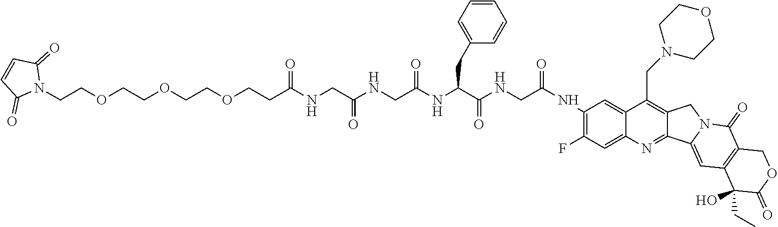 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN1CCOCC1] |
| DL: MC- GGFG- Compound 141 | 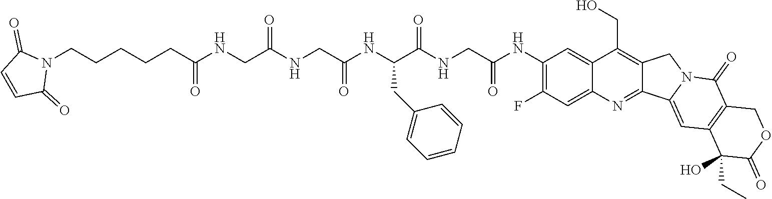 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCCCCN3C(=O)C=CC3=O)cc1c2CO] |
| DL: MC- VA- Compound 140 (DL: 201) | 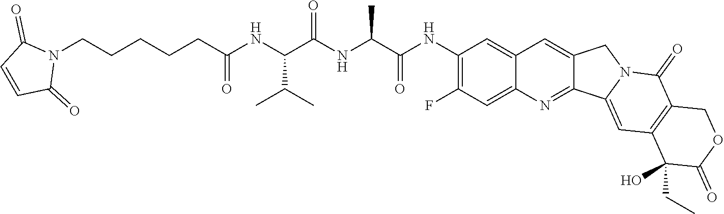 [SMILES: CC[C@@]1(O)C(=O)OCc2c1cc1n(c2=O)Cc2cc3cc(NC(=O)[C@H](C)NC(=O)[C@@H](NC(=O)CCCCCN4C(=O)C=CC4=O)C(C)C)c(F)cc3nc2-1] |
| DL: NHC- C-VA- Compound 140 | 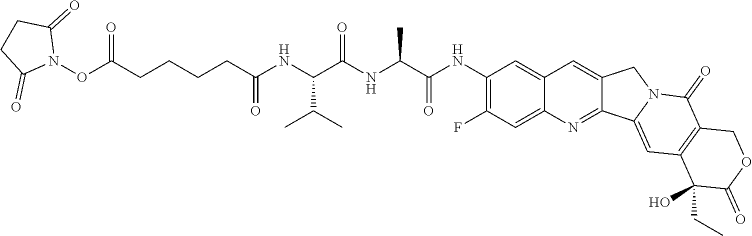 [SMILES: CC[C@@]1(O)C(=O)OCc2c1cc1n(c2=O)Cc2cc3cc(NC(=O)[C@H](C)NC(=O)[C@@H](NC(=O)CCCCC(=O)ON4C(=O)CCC4=O)C(C)C)c(F)cc3nc2-1] |
| DL: Azido- PEG8- VA- Compound 140 | 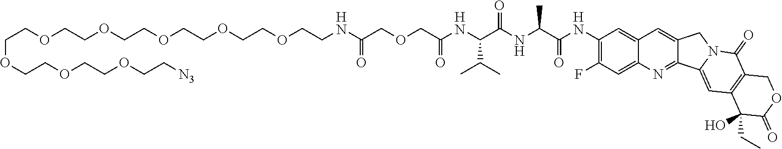 [SMILES: CC[C@@]1(O)C(=O)OCc2c1cc1n(c2=O)Cc2cc3cc(NC(=O)[C@H](C)NC(=O)[C@@H](NC(=O)COCC(=O)NCCOCCOCCOCCOCCOCCOCCOCCOCCN=[N+]=[N-])C(C)C)c(F)cc3nc2-1] |

TABLE 9
Exemplary drug-linker (DL) structures comprising camptothecin analogues of
Formula (1) with either a C7 or C10 linkage

| Name | Structure |
|---|---|
| DL: 200 | 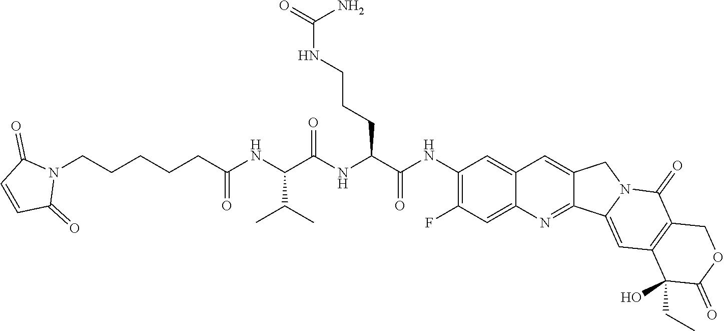 [SMILES: CC[C@@]1(O)C(=O)OCc2c1cc1n(c2=O)Cc2cc3cc(NC(=O)[C@H](CCCNC(N)=O)NC(=O)[C@@H](NC(=O)CCCCCN4C(=O)C=CC4=O)C(C)C)c(F)cc3nc2-1] |
| DL: 202 | 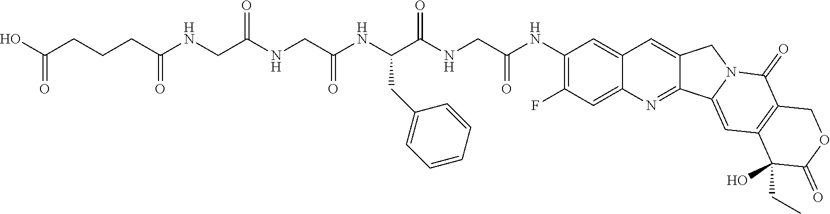 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)CCCC(=O)O)c(F)cc3nc2-1] |
| DL: 203 | 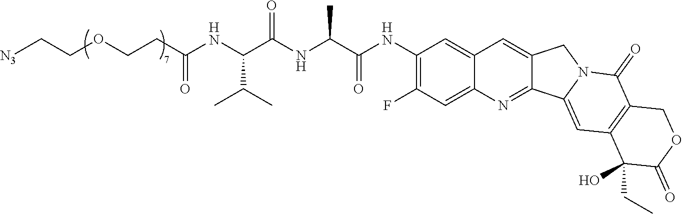 |
| DL: 204 | 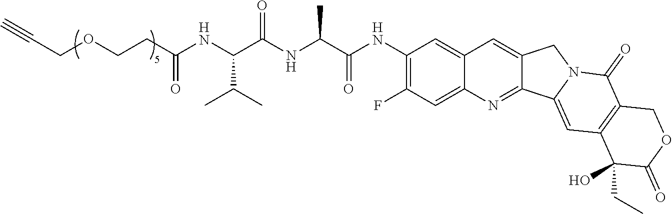 |
| DL: 205 | 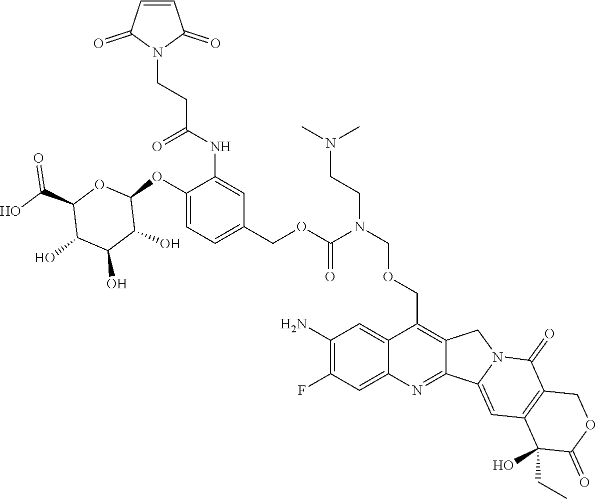 [SMILES: CC[C@@]1(O)C(=O)OCc2c1cc1n(c2=O)Cc2c-1nc1cc(F)c(N)cc1c2COCN(CCN(C)C)C(=O)OCc1ccc(O[C@@H]2O[C@H](C(=O)O)[C@@H](O)[C@H](O)[C@H]2O)c(NC(=O)CCN2C(=O)C=CC2=O)c1] |
| DL: 206 | 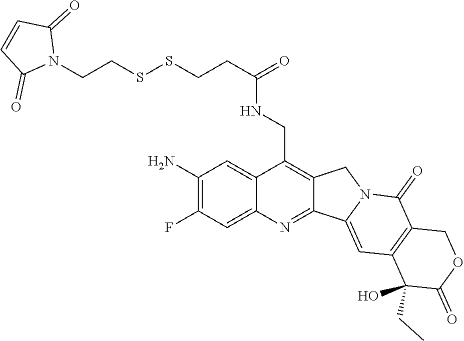 [SMILES: CC[C@@]1(O)C(=O)OCc2c1cc1n(c2=O)Cc2c-1nc1cc(F)c(N)cc1c2CNC(=O)CCSSCCN1C(=O)C=CC1=O] |
| DL: 207 | 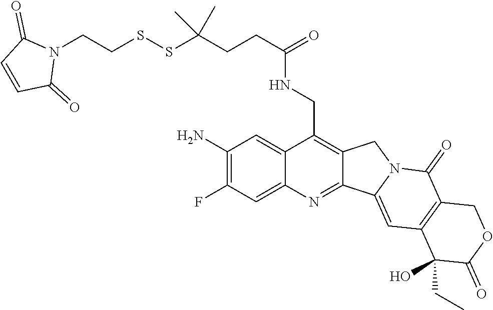 [SMILES: CC[C@@]1(O)C(=O)OCc2c1cc1n(c2=O)Cc2c-1nc1cc(F)c(N)cc1c2CNC(=O)CCC(C)(C)SSCCN1C(=O)C=CC1=O] |
| DL: 208 | 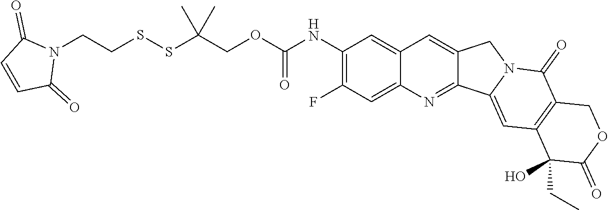 [SMILES: CC[C@@]1(O)C(=O)OCc2c1cc1n(c2=O)Cc2cc3cc(NC(=O)OCC(C)(C)SSCCN4C(=O)C=CC4=O)c(F)cc3nc2-1] |
| DL: 209 | 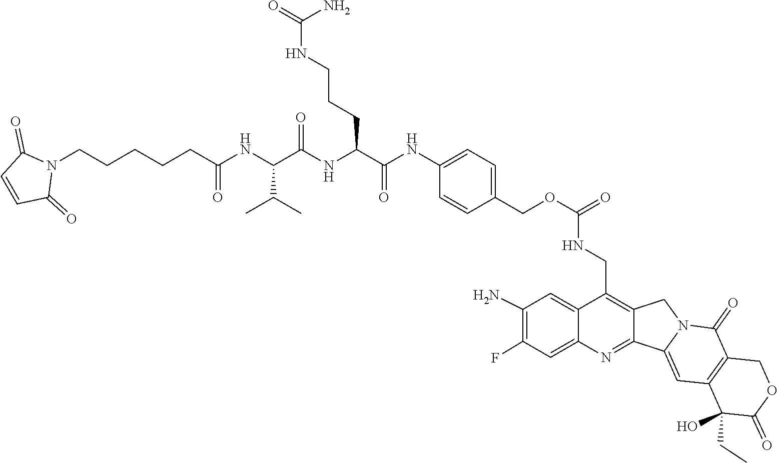 [SMILES: CC[C@@]1(O)C(=O)OCc2c1cc1n(c2=O)Cc2c-1nc1cc(F)c(N)cc1c2CNC(=O)OCc1ccc(NC(=O)[C@H](CCCNC(N)=O)NC(=O)[C@@H](NC(=O)CCCCCN2C(=O)C=CC2=O)C(C)C)cc1] |
| DL: 210 | 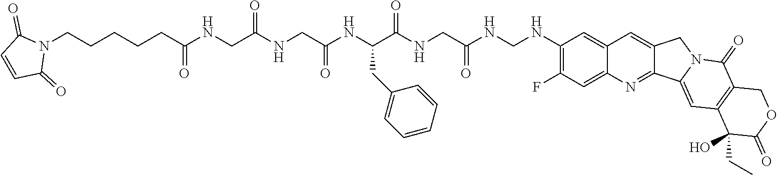 [SMILES: CC[C@@]1(O)C(=O)OCc2c1cc1n(c2=O)Cc2cc3cc(NCNC(=O)CNC(=O)[C@H](Cc4ccccc4)NC(=O)CNC(=O)CNC(=O)CCCCCN4C(=O)C=CC4=O)c(F)cc3nc2-1] |

TABLE 10
Exemplary conjugate (DC) structures comprising camptothecin analogues of
Formula (I) with a C7 linkage

| Name | Structure |
|---|---|
| DC: MT- GGFG- Compound 104 | 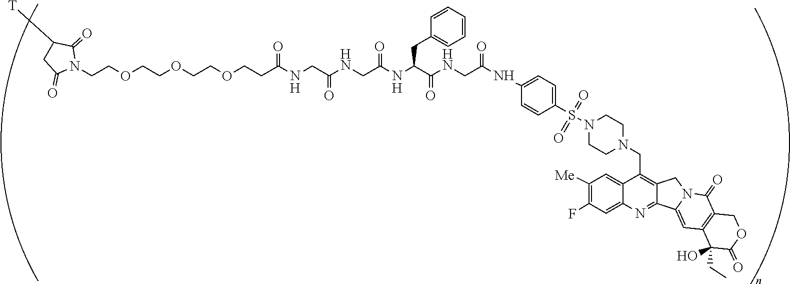 |
| DC: MT- GGFG- Compound 108 | 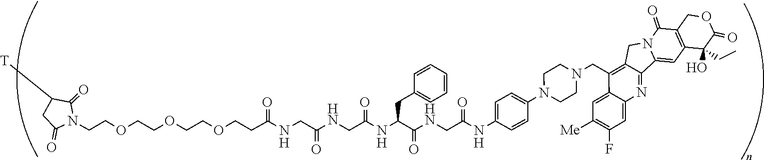 |
| DC: MT- GGFG- Compound 127 | 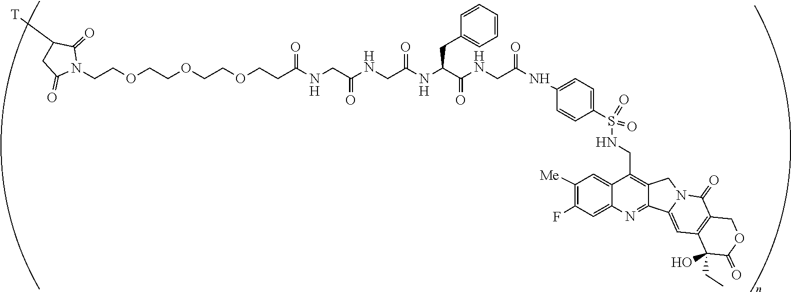 |
| DC: MT- GGFG- AM- Compound 136 | 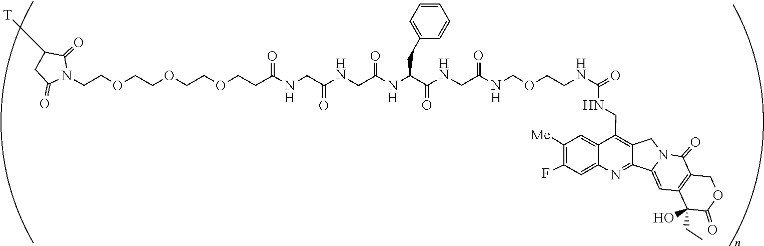 |
| DC: MT- GGFG- AM- Compound 139 | 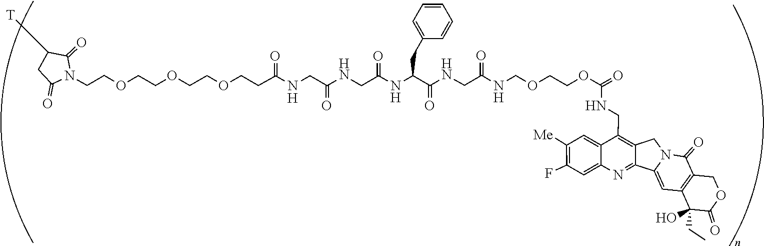 |
| DC: MT- GGFG- AM- Compound 129 | 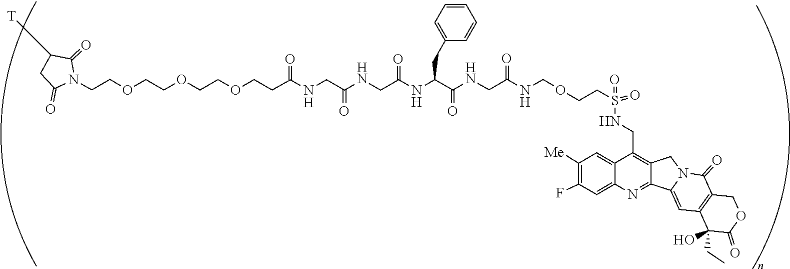 |
| DC: MT- GGFG- AM- Compound 113 | 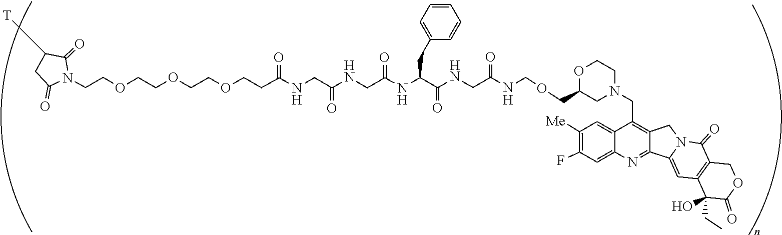 |
| DC: MT- GGFG- AM- Compound 141 | 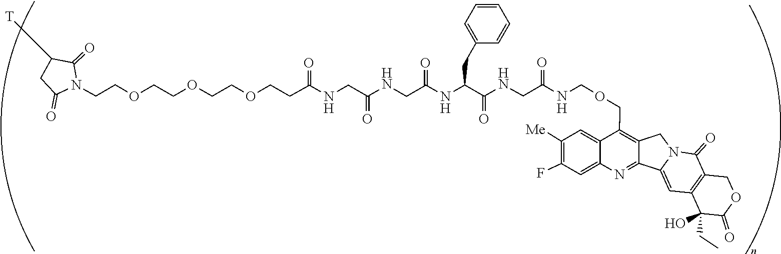 |
| DC: MC- GGFG- AM- Compound 141 | 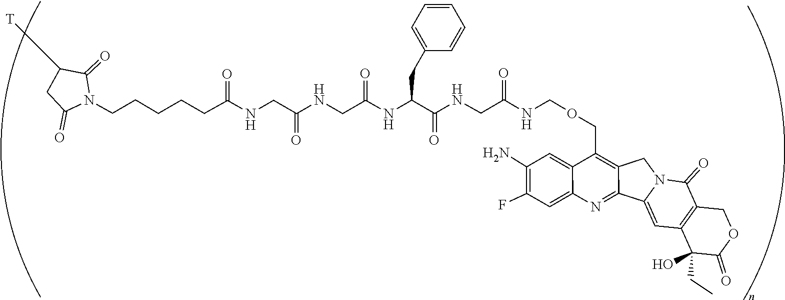 |
| DC: MT- GGFG- AM- Compound 117 | 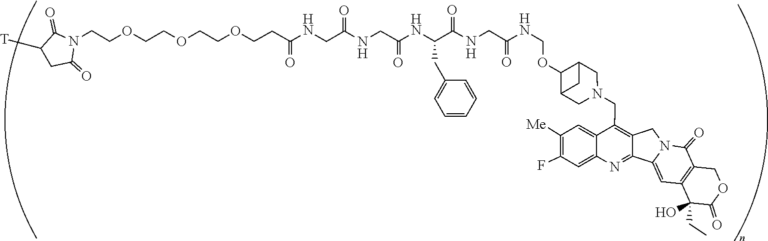 |
| DC: MT- GGFG- AM- Compound 118 | 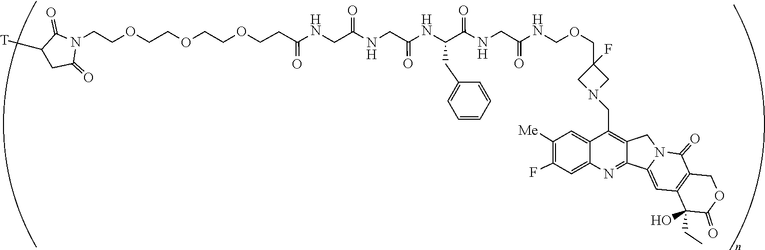 |
| DC: MC- GGFG- AM- Compound 139 | 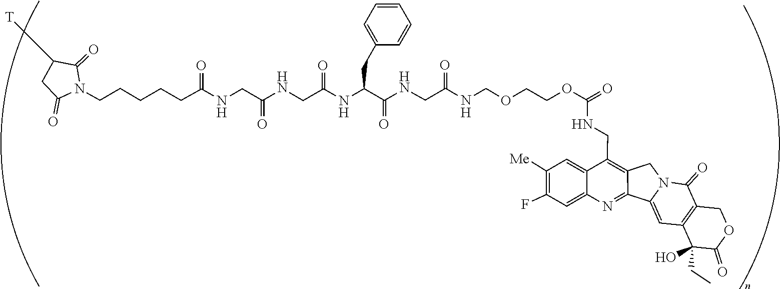 |
| DC: DIS- Compound 145 | 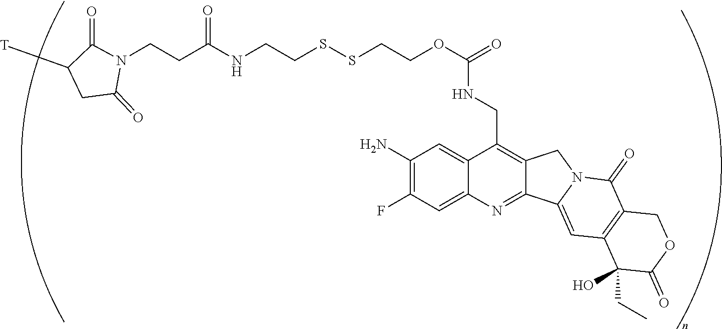 |

TABLE 11
Exemplary conjugate (DC) structures comprising camptothecin analogues of
Formula (I) with a C10 linkage

| Name | Structure |
|---|---|
| DC: MT- GGFG- Compound 140 | 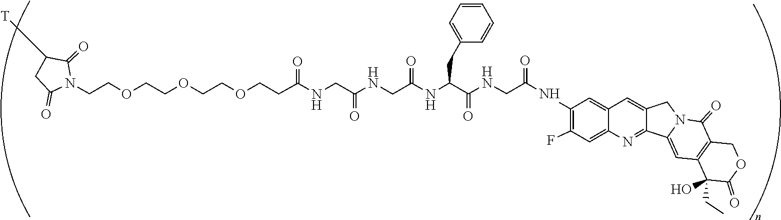 |
| DC: MT- GGFG- Compound 141 | 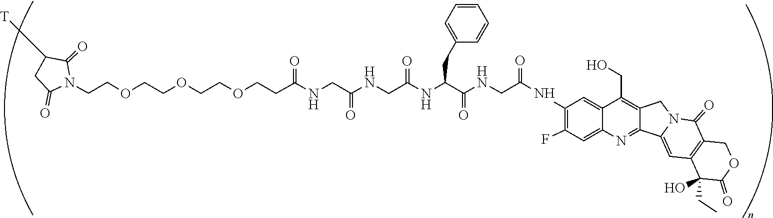 |
| DC: MT- GGFG- Compound 145 | 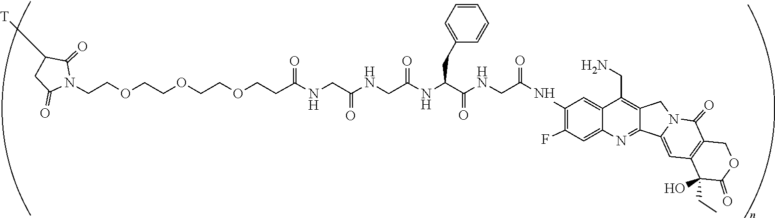 |
| DC: MT- GGFG- Compound 148 | 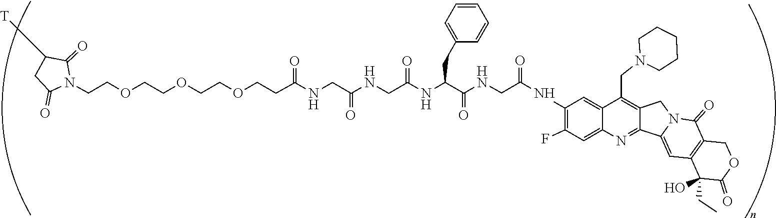 |
| DC: MT- GGFG- Compound 142 | 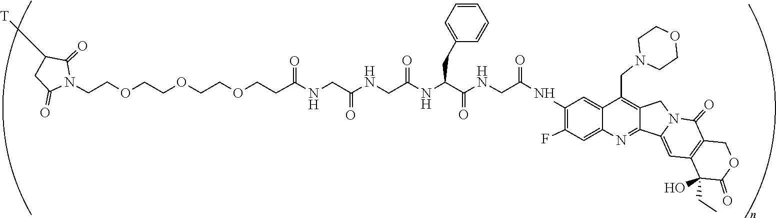 |
| DC: MC- GGFG- Compound 140 | 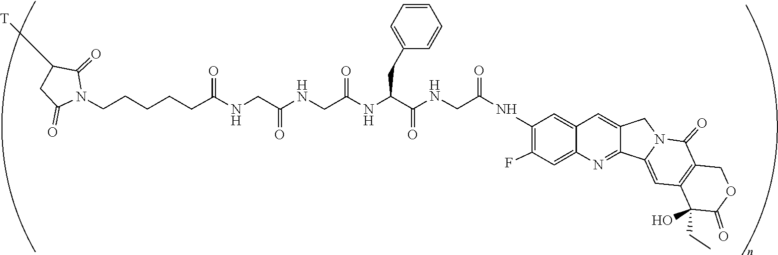 |
| DC: MC- GGFG- Compound 141 | 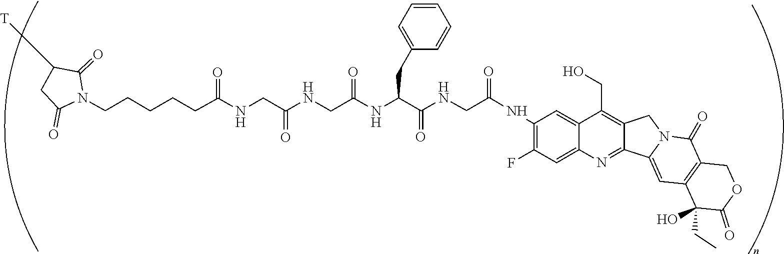 |
| DC: MC- VA- Compound 140 (DC: 301) | 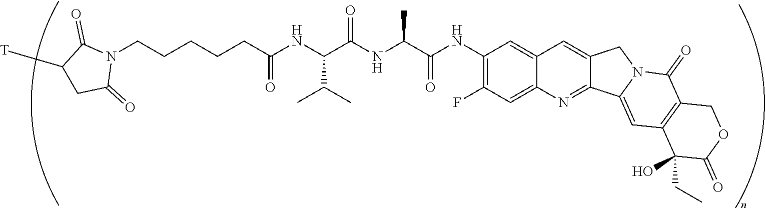 |
| DC: NHC- C-VA- Compound 140 | 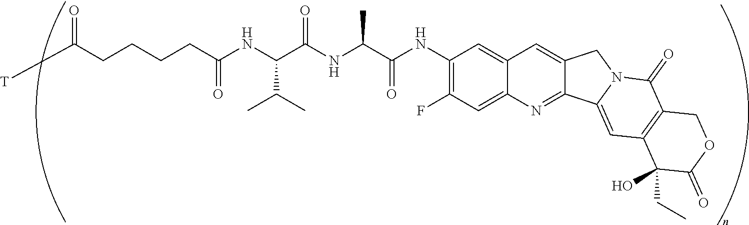 |
| DC: Azido- PEG8-VA- Compound 140 | 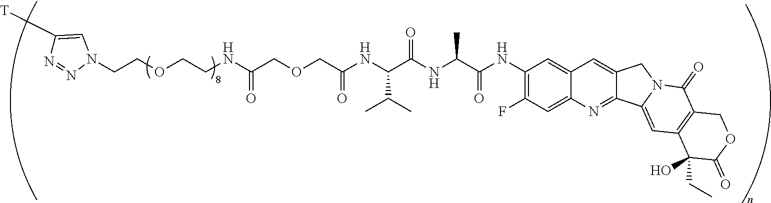 |

TABLE 12
Exemplary conjugate (DC) structures comprising camptothecin analogues of
Formula (1) with either a C7 or C10 linkage

| Name | Structure |
|---|---|
| DC: 300 | 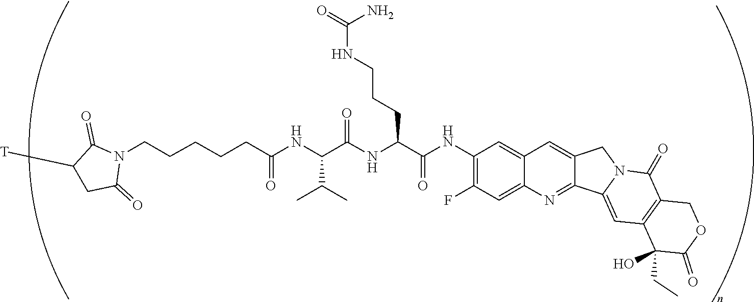 |
| DC: 302 | 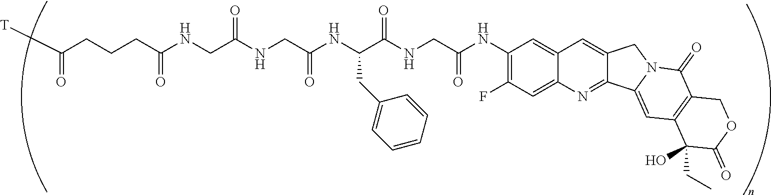 |
| DC: 303a | 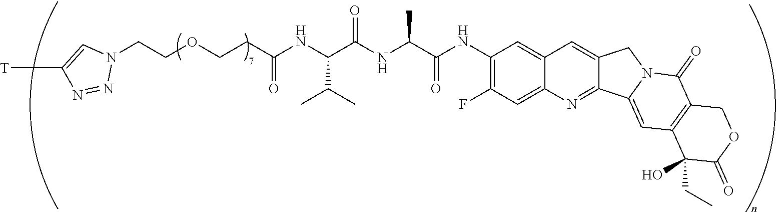 |
| DC: 303b | 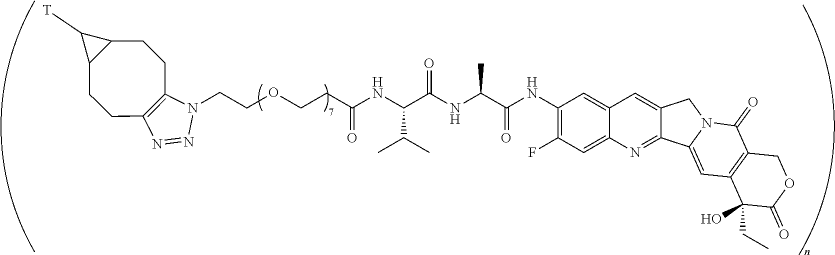 |
| DC: 304 | 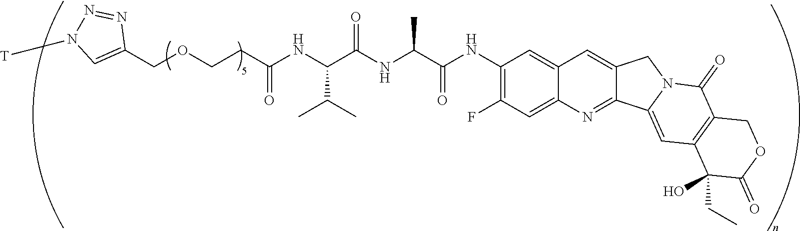 |
| DC: 305 | 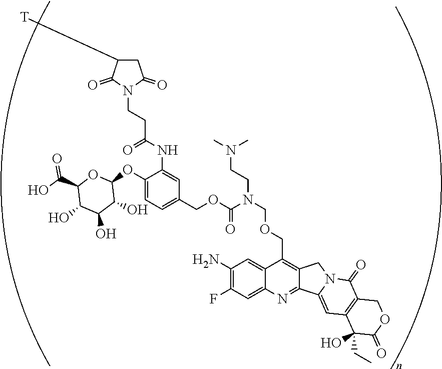 |
| DC: 306 | 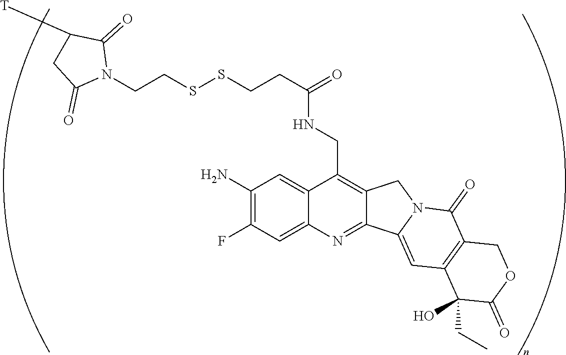 |
| DC: 307 | 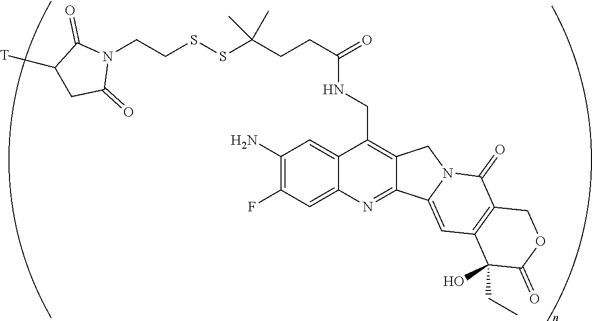 |
| DC: 308 | 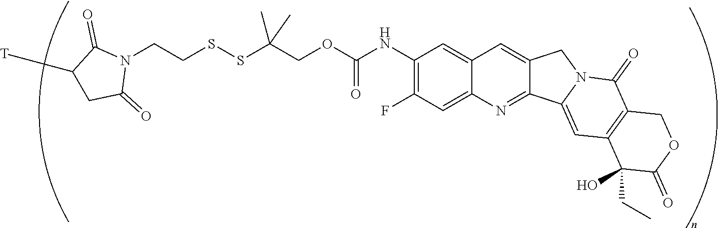 |
| DC: 309 | 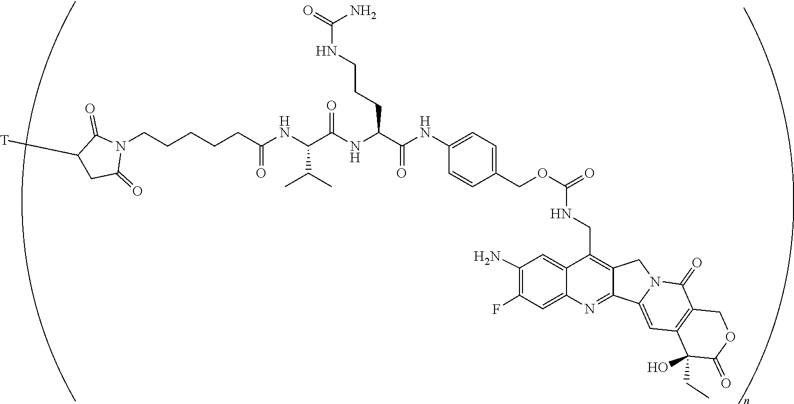 |
| DC: 310 | 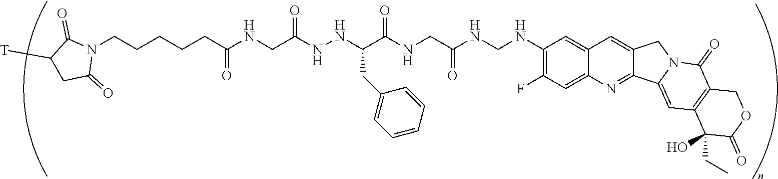 |

The following Examples are provided for illustrative purposes and are not intended to limit the scope of the invention in any way.

### Examples

Examples 1-3 below illustrate various methods of preparing camptothecin analogues of Formula (I). It is understood that one skilled in the art may be able to make these compounds by similar methods or by combining other methods known in the art. It is also understood that one skilled in the art would be able to make, using the methods described below or similar methods, other compounds of Formula (I) not specifically illustrated below by using the appropriate starting components and modifying the parameters of the synthesis as needed. In general, starting components may be obtained from commercial sources such as Sigma Aldrich (Merck KGaA), Alfa Aesar and Maybridge (Thermo Fisher Scientific Inc.), Matrix Scientific, Tokyo Chemical Industry Ltd. (TCI) and Fluorochem Ltd., or synthesized according to sources known to those skilled in the art (see, for example, March's Advanced Organic Chemistry: Reactions, Mechanisms, and Structure, 7th edition, John Wiley & Sons, Inc., 2013) or prepared as described herein.

### ABBREVIATIONS

The following abbreviations are used throughout the Examples section: BCA: bicinchonic acid; Boc: di-tert-butyl dicarbonate; CE-SDS: capillary electrophoresis sodium dodecyl sulfate; DCM: dichloromethane; DTPA: diethylenetriamine pentaacetic acid; DIPEA: N,N-diisopropylethylamine; DMF: dimethylformamide; DMM™: (4-(4,6-dimethoxy-1,3,5-triazin-2-yl)-4-methyl-morpholiniurn chloride; EDC: 1-ethyl-3-(3-dimethylaminopropyl)carbodiimide; Fmoc: fluorenylmethyloxycarbonyl; HATU: hexafluorophosphate azabenzotriazole tetramethyl uronium; HIC: hydrophobic interaction chromatography; HOAt: 1-hydroxy-7-azabenzotriazole; HPLC: high-performance liquid chromatography; LC/MS: liquid chromatography mass spectrometry; MC: maleimidocaproyl; MT: maleimidotriethylene glycolate; NMM: N-methylmorpholine; PNP: p-nitrophenol; RP-UPLC-MS: reversed-phase ultra-high performance chromatography mass spectrometry; SEC: size exclusion chromatography; TCEP: tris(2-carboxyethyl) phosphine; Tfp: tetrafluorophenyl; TLC: thin layer chromatography; TFA: trifluoracetic acid.

### General Chemistry Procedures

### General Procedure 1: Conversion of Chloride to Amine

To a stirring solution of chloride compound in dimethylformamide (0.05-0.1 M) was added the appropriate secondary amine (3 eq.). Upon completion (determined by LC/MS, typically 1-3 h), the reaction mixture was purified by reverse-phase HPLC to provide the desired product after lyophilization.

### General Procedure 2: Conversion of Amine to Amide

To a stirring solution of amine compound in dimethylformamide (0.05-0.1 M) was added triethylamine (1.2 eq.), the appropriate carboxylic acid (1.1 eq.) followed by a solution of DMM™ (2 eq.) in water (1 M). Upon completion (determined by LC/MS, typically 16 h), the reaction mixture was purified by reverse-phase HPLC to provide the desired product after lyophilization.

### General Procedure 3: Conversion of Amine to Sulfonamide

To a stirring solution of amine compound in dimethylformamide (0.05-0.1 M) was added DIPEA (3 eq.) followed by the appropriate sulfonyl chloride. Upon completion (determined by LC/MS, typically 16 h), the reaction mixture was purified by reverse-phase HPLC to provide the desired product after lyophilization.

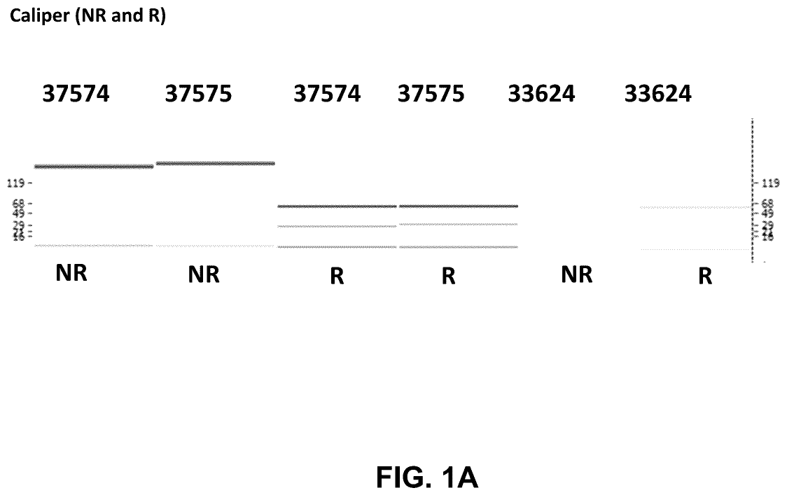

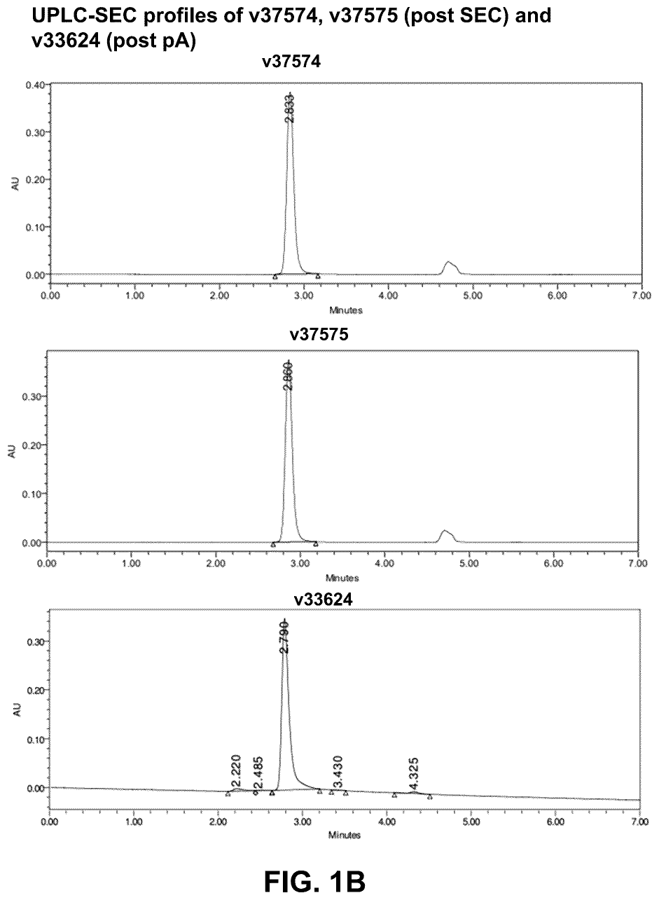

### General Procedure 4: 2-Step Conversion of Amine to Urea (Synthetic Scheme IV; FIG. 1D)

Step 1: To a stirring solution of amine compound in dichloromethane or dimethylformamide (0.05-0.1 M) was added p-nitrophenyl carbonate (1 eq.) then triethylamine (2 eq.). Upon completion (determined by LC/MS typically 1-4 h), the reaction mixture was concentrated to dryness then purified by reverse-phase HPLC to provide the desired PNP-carbamate intermediate after lyophilization. This intermediate can be used to generate a single analog or be divided into multiple batches in order to generate multiple analogs in the second step. Step 2: To the PNP-carbamate intermediate in dimethylformamide (0.1-0.2 M) was added the appropriate primary amine (3 eq.). Upon completion (determined by LC/MS, typically 1 h), the reaction mixture was purified by reverse-phase HPLC to provide the desired product after lyophilization.

### General Procedure 5: Conversion of Amine to Carbamate

To a stirring solution of amine compound in dichloromethane or dimethylformamide (0.05-0.1 M) was added p-nitrophenyl carbonate (1 eq.) then triethylamine (2 eq.). Upon completion (determined by LC/MS, typically 1-4 h), the appropriate alcohol was added to the resultant PNP-carbamate intermediate. Upon completion (determined by LC/MS, typically 1-16 h), the reaction mixture was purified by reverse-phase HPLC to provide the desired product after lyophilization.

### General Procedure 6: Removal of Boc Protecting Group

To a stirring solution of the Boc-protected amine compound in dichloromethane (0.1 M) was added TFA (20% by volume). Upon completion (determined by LC/MS, typically 1 h), the reaction mixture was concentrated in vacuo to provide a crude solid or was purified as described in General Procedure 9.

### General Procedure 7: Copper-Mediated Amide Coupling

To a rapidly stirring solution of Boc-GGFG-OH (3 eq.) and HOAt (3 eq.) in a 10% v/v mixture of dimethyl formamide in dichloromethane (0.02 M) was added EDC (HCl salt, 3 eq.). After 5 min, a solution of the amine containing payload (1 eq.) in a 10% v/v mixture of dimethyl formamide in dichloromethane (0.02 M) was added, followed immediately by the addition of CuCl2 (4 eq.). Upon completion (determined by LC/MS, typically 1-16 h), the reaction mixture was concentrated in vacuo to provide a crude solid or was purified by preparative HPLC to provide the desired product after lyophilization.

### General Procedure 8: MT Installation

To a stirring solution of amine compound (1 eq.) in dimethylformamide (—0.02 M) was added a solution of MT-OTfp (1.2-1.5 eq.) in acetonitrile (˜0.02 M) then DIPEA (10 μL, 4 eq.). Upon completion (determined by LC/MS, typically 1-16 h), the reaction mixture was concentrated in vacuo to provide a crude solid which was purified by preparative HPLC to provide the desired product after lyophilization.

### General Procedure 9: Compound Purification

Flash Chromatography: Crude reaction products were purified with Biotage® Snap Ultra columns (10, 25, 50, or 100 g) (Biotage, Charlotte, NC), eluting with linear gradients of ethyl acetate/hexanes or methanol/dichloromethane on a Biotage® Isolera™ automated flash system (Biotage, Charlotte, NC). Alternatively, reverse-phase flash purification was conducting using Biotage® Snap Ultra C₁₈ columns (12, 30, 60, or 120 g), eluting with linear gradients of 0.1% TFA in acetonitrile/0.1% TFA in water. Purified compounds were isolated by either removal of organic solvents by rotavap or lyophilization of acetonitrile/water mixtures.

Preparative HPLC: Reverse-phase HPLC of crude compounds was performed using a Luna® 5-μm C18 100 Å (150×30 mm) column (Phenomenex, Torrance, CA) on an Agilent 1260 Infinity II preparative LC/MSD system (Agilent Technologies, Inc., Santa Clara, CA), and eluting with linear gradients of 0.1% TFA in acetonitrile/0.1% TFA in water. Purified compounds were isolated by lyophilization of acetonitrile/water mixtures.

### General Procedure 10: Compound Analysis

LC/MS: Reactions were monitored for completion and purified compounds were analyzed using a Kinetex® 2.6-μm C18 100 Å(30×3 mm) column (Phenomenex, Torrance, CA) on an Agilent 1290 HPLC/6120 single quad LC/MS system (Agilent Technologies, Inc., Santa Clara, CA), eluting with a 10 to 100% linear gradient of 0.1% formic acid in acetonitrile/0.1% formic acid in water.

NMR: ¹H NMR spectra were collected with a Bruker AVANCE III 300 Spectrometer (300 MHz) (Bruker Corporation, Billerica, MA). Chemical shifts are reported in parts per million (ppm).

### Example 1: Preparation of Camptothecin Analogues Having Methyl at the C₁₀ Position

### 1.1: (S)-11-(chloromethyl)-4-ethyl-8-fluoro-4-hydroxy-9-methyl-1,12-dihydro-14H-pyrano[3′,4′:6,7]indolizino[1,2-b]quinoline-3,14(4H)-dione (Compound 1.1)

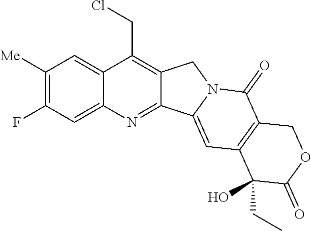 [SMILES: CC[C@@]1(O)C(=O)OCc2c1cc1n(c2=O)Cc2c-1nc1cc(F)c(C)cc1c2CCl]

The title compound was prepared according to the procedure provided in Li, et al., 2019, ACS Med. Chem. Lett., 10(10): 1386-1392.

### 1.2: (S)-11-(aminomethyl)-4-ethyl-8-fluoro-4-hydroxy-9-methyl-1,12-dihydro-14H-pyrano[3′,4′ 6,7]indolizino[1,2-b]quinoline-3,14(4H)-dione (Compound 1.2)

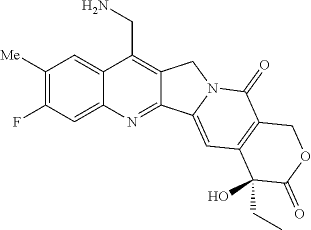 [SMILES: CC[C@@]1(O)C(=O)OCc2c1cc1n(c2=O)Cc2c-1nc1cc(F)c(C)cc1c2CN]

The title compound was prepared according to the procedure provided in Li, et al., 2019, ACS Med. Chem. Lett., 10(10): 1386-1392.

### 1.3: (S)-4-ethyl-8-fluoro-4-hydroxy-9-methyl-11-(morpholinomethyl)-1,12-dihydro-14H-pyrano[3′,4′ 6,7]indolizino[1,2-b]quinoline-3,14(4H)-dione (Compound 100)

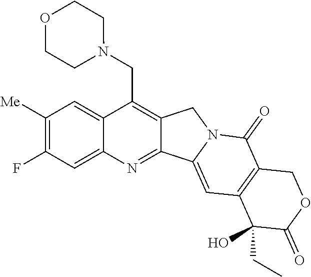 [SMILES: CC[C@@]1(O)C(=O)OCc2c1cc1n(c2=O)Cc2c-1nc1cc(F)c(C)cc1c2CN1CCOCC1]

The title compound was prepared according to General Procedure 1 starting from Compound 1.1 (10 mg) and morpholine. Preparative HPLC purification was accomplished as described in General Procedure 9, eluting with a 20 to 60% CH₃CN/H₂O+0.1% TFA gradient to give the title compound as an off-white solid (TFA salt, 3.6 mg, 26% yield).

LC/MS: Calc'd m/z=479.2 for C₂₆H26FN305, found [M+H]+=480.4.

¹H NMR (300 MHz, CDCl3) δ 8.20 (d, J=8.0 Hz, 1H), 7.82 (d, J=10.4 Hz, 1H), 7.67 (s, 1H), 5.77 (d, J=16.4 Hz, 1H), 5.42 (s, 2H), 5.33 (d, J=16.4 Hz, 1H), 4.26 (s, 2H), 3.81 (t, J=4.7 Hz, 4H), 2.82-2.76 (m, 4H), 2.57 (d, J=1.7 Hz, 3H), 1.99-1.82 (m, 2H), 1.06 (t, J=7.4 Hz, 3H).

### 1.4: (S)-4-ethyl-8-fluoro-4-hydroxy-9-methyl-11-((4-(phenylsulfonyl)piperazin-1-yl)methyl)-1,12-dihydro-14H-pyrano[3′,4′: 6,7]indolizino[1,2-b]quinoline-3,14(4H)-dione (Compound 102)

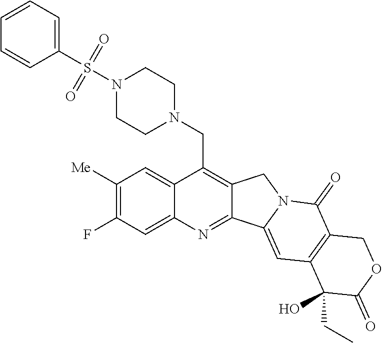 [SMILES: CC[C@@]1(O)C(=O)OCc2c1cc1n(c2=O)Cc2c-1nc1cc(F)c(C)cc1c2CN1CCN(S(=O)(=O)c2ccccc2)CC1]

The title compound was prepared according to General Procedure 1 starting from Compound 1.1 (10 mg) and 1-(phenylsulfonyl)piperazine. Preparative HPLC purification was accomplished as described in General Procedure 9, eluting with a 20 to 60% CH₃CN/H₂O+0.1% TFA gradient to give the title compound as an off-white solid (TFA salt, 3.6 mg, 21% yield).

LC/MS: Calc'd m/z=618.2 for C₃₂H31FN406, found [M+H]+=619.4.

¹H NMR (300 MHz, CDCl3) δ 8.07 (d, J=7.9 Hz, 1H), 7.88-7.44 (m, 7H), 5.73 (d, J=16.4 Hz, 1H), 5.33 (s, 2H), 5.33-5.26 (m, 1H), 4.19 (s, 2H), 3.12 (s, 4H), 2.80 (s, 4H), 2.54 (s, 3H), 1.90 (dt, J=11.6, 7.0 Hz, 2H), 1.04 (t, J=7.3 Hz, 3H).

### 1.5: (S)-11-((4-((4-aminophenyl)sulfonyl)piperazin-1-yl)methyl)-4-ethyl-8-fluoro-4-hydroxy-9-methyl-1,12-dihydro-14H-pyrano[3′,4′:6,7]indolizino[1,2-b]quinoline-3,14(4H)-dione (Compound 104)

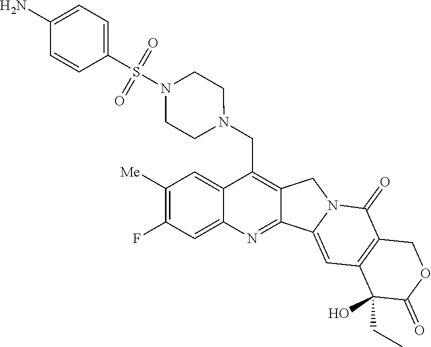 [SMILES: CC[C@@]1(O)C(=O)OCc2c1cc1n(c2=O)Cc2c-1nc1cc(F)c(C)cc1c2CN1CCN(S(=O)(=O)c2ccc(N)cc2)CC1]

The title compound was prepared according to General Procedure 1 starting from Compound 1.1 (10 mg) and 4-(piperazin-1-ylsulfonyl)aniline. Preparative HPLC purification was accomplished as described in General Procedure 9, eluting with a 20 to 60% CH₃CN/H₂O+0.1% TFA gradient to give the title compound as an off-white solid (TFA salt, 4.7 mg, 27% yield).

LC/MS: Calc'd m/z=633.2 for C₃₂H₃₂FN₅O₆, found [M+H]⁺=634.4.

¹H NMR (300 MHz, MeOD) δ 8.32 (d, J=8.0 Hz, 1H), 7.85 (d, J=10.5 Hz, 1H), 7.65 (s, 1H), 7.46 (d, J=8.7 Hz, 2H), 6.74 (d, J=8.7 Hz, 2H), 5.61 (d, J=16.5 Hz, 1H), 5.44 (s, 2H), 5.41 (d, J=16.5 Hz, 1H), 4.51 (s, 2H), 3.22-3.07 (m, 8H), 2.58 (s, 3H), 2.03-1.93 (m, 2H), 1.02 (t, J=7.3 Hz, 3H).

### 1.6: (S)-4-ethyl-8-fluoro-4-hydroxy-9-methyl-11-((4-methylpiperazin-1-yl)methyl)-1,12-dihydro-14H-pyrano[3′,4′:6,7]indolizino[1,2-b]quinoline-3,14(4H)-dione (Compound 106)

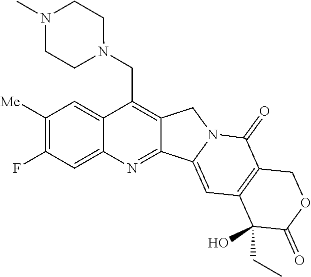 [SMILES: CC[C@@]1(O)C(=O)OCc2c1cc1n(c2=O)Cc2c-1nc1cc(F)c(C)cc1c2CN1CCN(C)CC1]

The title compound was prepared according to General Procedure 1 starting from Compound 1.1 (10 mg) and N-methylpiperazine. Preparative HPLC purification was accomplished as described in General Procedure 9, eluting with a 20 to 50% CH₃CN/H₂O+0.1% TFA gradient to give the title compound as an off-white solid (TFA salt, 3.6 mg, 25% yield).

LC/MS: Calc'd m/z=492.2 for C₂₇H₂₉FN404, found [M+H]⁺=493.4.

### 1.7. (S)-11-((4-(4-aminophenyl)piperazin-1-yl)methyl)-4-ethyl-8-fluoro-4-hydroxy-9-methyl-1,12-dihydro-14H-pyrano[3′,4′: 6,7]indolizino[1,2-b]quinoline-3,14(4H)-dione (Compound 108)

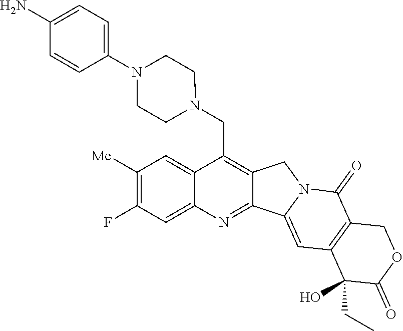 [SMILES: CC[C@@]1(O)C(=O)OCc2c1cc1n(c2=O)Cc2c-1nc1cc(F)c(C)cc1c2CN1CCN(c2ccc(N)cc2)CC1]

The title compound was prepared according to General Procedure 1 starting from Compound 1.1 (10 mg) and 4-(piperazin-1-yl)aniline. Preparative HPLC purification was accomplished as described in General Procedure 9, eluting with a 20 to 50% CH₃CN/H₂O+0.1% TFA gradient to give the title compound as an off-white solid (TFA salt, 3.7 mg, 23% yield).

LC/MS: Calc'd m/z=569.2 for C₃₂H₃₂FN₅O₄, found [M+H]⁺=570.4.

¹H NMR (300 MHz, MeOD) δ 8.39 (d, J=8.1 Hz, 1H), 7.79 (d, J=10.6 Hz, 1H), 7.21 (d, J=9.0 Hz, 2H), 7.14 (d, J=9.0 Hz, 2H), 5.62 (d, J=16.4 Hz, 1H), 5.49 (s, 2H), 5.41 (d, J=16.4 Hz, 1H), 4.45 (s, 2H), 3.44-3.38 (m, 4H), 3.06-3.00 (m, 4H), 2.58 (d, J=1.8 Hz, 3H), 2.00-1.89 (m, 2H), 1.03 (t, J=7.3 Hz, 3H).

### 1.8: (S)-4-ethyl-8-fluoro-4-hydroxy-9-methyl-11-(piperidin-1-ylmethyl)-1,12-dihydro-14H-pyrano[3′,4′:6,7]indolizino[1,2-b]quinoline-3,14(4H)-dione (Compound 110)

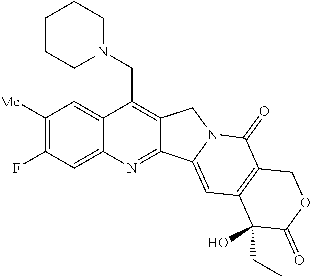 [SMILES: CC[C@@]1(O)C(=O)OCc2c1cc1n(c2=O)Cc2c-1nc1cc(F)c(C)cc1c2CN1CCCCC1]

The title compound was prepared according to General Procedure 1 starting from Compound 1.1 (10 mg) and piperidine. Preparative HPLC purification was accomplished as described in General Procedure 9, eluting with a 10 to 60% CH₃CN/H₂O+0.1% TFA gradient to give the title compound as an off-white solid (TFA salt, 1.5 mg, 11% yield).

LC/MS: Calc'd m/z=477.2 for C₂₇H₂₈FN304, found [M+H]⁺=478.2.

¹H NMR (300 MHz, MeOD) δ 8.34 (d, J=7.6 Hz, 1H), 7.94 (d, J=10.3 Hz, 1H), 7.70 (s, 1H), 5.63 (d, J=16.4 Hz, 1H), 5.52 (s, 2H), 5.44 (d, J=16.5 Hz, 1H), 4.99 (s, 2H), 3.73-3.46 (m, 4H), 2.64 (s, 3H), 2.03-1.90 (m, 2H), 1.90-1.84 (m, 6H), 1.03 (t, J=7.4 Hz, 3H).

### 1.9: tert-butyl (S)-4-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)piperazine-1-carboxylate (Compound 111)

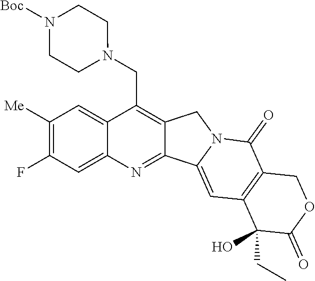 [SMILES: CC[C@@]1(O)C(=O)OCc2c1cc1n(c2=O)Cc2c-1nc1cc(F)c(C)cc1c2CN1CCN(C(=O)OC(C)(C)C)CC1]

The title compound was prepared according to General Procedure 1 starting from Compound 1.1 (10 mg) and tert-butyl piperazine-1-carboxylate. Preparative HPLC purification was accomplished as described in General Procedure 9, eluting with a 10 to 60% CH₃CN/H₂O+0.1% TFA gradient to give the title compound as an off-white solid (TFA salt, 6.6 mg, 40% yield).

LC/MS: Calc'd m/z=578.2 for C₃₁H₃₅FN406, found [M+H]⁺=579.4.

### 1.10: (S)-4-ethyl-8-fluoro-4-hydroxy-9-methyl-11-(piperazin-1-ylmethyl)-1,12-dihydro-14H-pyrano[3′,4′:6,7]indolizino[1,2-b]quinoline-3,14(4H)-dione (Compound 112)

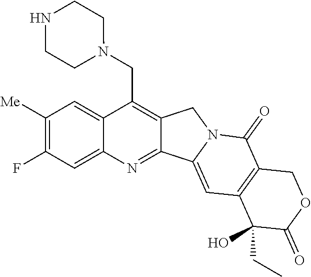 [SMILES: CC[C@@]1(O)C(=O)OCc2c1cc1n(c2=O)Cc2c-1nc1cc(F)c(C)cc1c2CN1CCNCC1]

The title compound was prepared according to General Procedure 6 starting from Compound 111 (5.0 mg) to give the title compound as an off-white solid (TFA salt, 4.4 mg).

LC/MS: Calc'd m/z=478.2 for C₂₆H₂₇FN404, found [M+H]⁺=479.2.

### 1.11: (S)-4-ethyl-8-fluoro-4-hydroxy-11-(((R)-2-(hydroxymethyl)morpholino)methyl)-9-methyl-1,12-dihydro-14H-pyrano[3′,4′:6,7]indolizino[1,2-b]quinoline-3,14(4H)-dione (Compound 113)

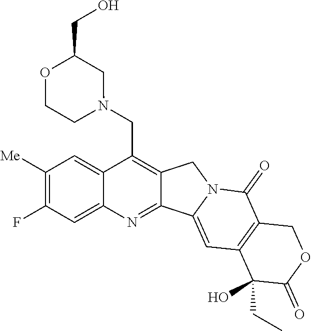 [SMILES: CC[C@@]1(O)C(=O)OCc2c1cc1n(c2=O)Cc2c-1nc1cc(F)c(C)cc1c2CN1CCO[C@@H](CO)C1]

The title compound was prepared according to General Procedure 1 starting from Compound 1.1 (10 mg) and (R)-morpholin-2-yl methanol. Preparative HPLC purification was accomplished as described in General Procedure 9, eluting with a 10 to 60% CH₃CN/H₂O+0.1% TFA gradient to give the title compound as an off-white solid (TFA salt, 4.6 mg, 32% yield).

LC/MS: Calc'd m/z=509.2 for C₂₇H₂₈FN306, found [M+H]⁺=510.4.

### 1.12: (4S)-4-ethyl-8-fluoro-4-hydroxy-11-((3-(hydroxymethyl)thiomorpholino)methyl)-9-methyl-1,12-dihydro-14H-pyrano[3′,4′:6,7]indolizino[1,2-b]quinoline-3,14(4H)-dione (Compound 114)

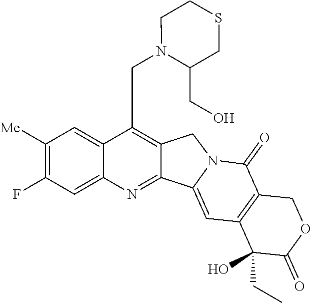 [SMILES: CC[C@@]1(O)C(=O)OCc2c1cc1n(c2=O)Cc2c-1nc1cc(F)c(C)cc1c2CN1CCSCC1CO]

The title compound was prepared according to General Procedure 1 starting from Compound 1.1 (10 mg) and thiomorpholin-3-ylmethanol. Preparative HPLC purification was accomplished as described in General Procedure 9, eluting with a 10 to 60% CH₃CN/H₂O+0.1% TFA gradient to give the title compound as an off-white solid (TFA salt, 1.5 mg, 12% yield).

LC/MS: Calc'd m/z=525.6 for C₂₇H₂₈FN305S, found [M+H]⁺=526.5.

¹H NMR (300 MHz, 10% D₂O/CD3CN) 8.36 (d, J=8.1 Hz, 1H), 7.83 (d, J=10.7 Hz, 1H), 7.50 (s, 1H), 5.57 (d, J=16.4 Hz, 1H), 5.52-5.29 (m, 3H), 5.02 (d, J=14.6 Hz, 1H), 4.71-4.54 (m, 1H), 4.27 (dd, J=12.4, 5.0 Hz, 1H), 3.98 (dd, J=12.3, 3.4 Hz, 1H), 3.55 (s, 1H), 3.30-3.03 (m, 4H) 2.97-2.72 (m, 3H), 2.62 (s, 1H), 2.55 (s, 3H), 0.95 (t, J=7.4 Hz, 3H).

### 1.13: (4S)-4-ethyl-8-fluoro-4-hydroxy-11-((4-(hydroxymethyl)-2-oxa-5-azabicyclo[2.2.1]heptan-5-yl)methyl)-9-methyl-1,12-dihydro-14H-pyrano[3′,4′:6,7]indolizino[1,2-b]quinoline-3,14(4H)-dione (Compound 115)

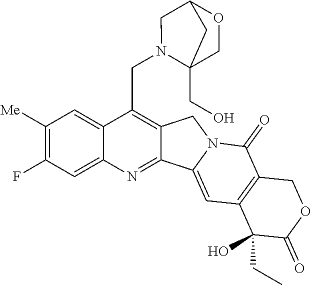 [SMILES: CC[C@@]1(O)C(=O)OCc2c1cc1n(c2=O)Cc2c-1nc1cc(F)c(C)cc1c2CN1CC2CC1(CO)CO2]

The title compound was prepared according to General Procedure 1 starting from Compound 1.1 (10 mg) and 2-oxa-5-azabicyclo[2.2.1]heptan-4-yl methanol. Preparative HPLC purification was accomplished as described in General Procedure 9, eluting with a 10 to 60% CH₃CN/H₂O+0.1% TFA gradient to give the title compound as an off-white solid (TFA salt, 3.5 mg, 29% yield).

LC/MS: Calc'd m/z=521.5 for C₂₈H₂₈FN306, found [M+H]⁺=522.5.

¹H NMR (300 MHz, 10% D₂O/CD3CN) 6 8.36 (d, J=7.9 Hz, 1H), 7.86 (dd, J=10.6, 5.0 Hz, 1H), 7.50 (d, J=1.8 Hz, 1H), 5.63-5.49 (m, 2H), 5.37 (dd, J=17.8, 14.1 Hz, 2H), 5.05 (s, 2H), 4.63 (d, J=2.5 Hz, 1H), 4.55 (d, J=10.7 Hz, 1H), 4.33 (s, 2H), 3.92 (d, J=10.7 Hz, 1H), 3.36 (s, 2H), 2.57 (s, 3H), 2.41-2.13 (m, 2H), 1.97-1.85 (m, 2H), 0.95 (t, J=7.4 Hz, 3H).

### 1.14: (4S)-4-ethyl-8-fluoro-4-hydroxy-11-((3-(hydroxymethyl)-1,1-dioxidothiomorpholino) methyl)-9-methyl-1,12-dihydro-14H-pyrano[3′,4′ 6,7]indolizino[1,2-b]quinoline-3,14(4H)-dione (Compound 116)

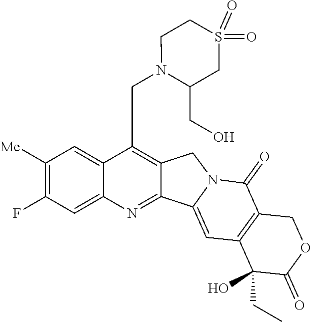 [SMILES: CC[C@@]1(O)C(=O)OCc2c1cc1n(c2=O)Cc2c-1nc1cc(F)c(C)cc1c2CN1CCS(=O)(=O)CC1CO]

The title compound was prepared according to General Procedure 1 starting from Compound 1.1 (10 mg) and 3-(hydroxymethyl)-1)⁶-thiomorpholine-1,1-dione. Purification was accomplished as described in General Procedure 9, eluting with a 10 to 60% CH₃CN/H₂O+0.1% TFA gradient to give the title compound as an off-white solid (TFA salt, 0.2 mg, 2% yield).

LC/MS: Calc'd m/z=557.6 for C₂₇H₂₈FN₃O₇S, found [M+H]⁺=558.4.

¹H NMR (300 MHz, 10% D₂O/CD3CN) 6 8.44 (d, J=8.2 Hz, 1H), 7.80 (d, J=11.0 Hz, 1H), 7.50 (s, 1H), 5.58 (d, J=16.5 Hz, 1H), 5.45-5.26 (m, 3H), 4.60 (d, J=14.9 Hz, 1H), 4.33 (d, J=14.7 Hz, 1H), 3.88 (d, J=4.8 Hz, 2H), 3.41-2.85 (m, 4H), 2.53 (s, 2H), 2.19 (p, J=2.5 Hz, 2H), 1.74 (p, J=2.5 Hz, 2H), 1.27 (s, 2H), 0.95 (t, J=7.4 Hz, 3H).

### 1.15: (4S)-4-ethyl-8-fluoro-4-hydroxy-11-((6-hydroxy-3-azabicylo[3.1.1]heptan-3-yl)methyl)-9-methyl-1,12-dihydro-14H-pyrano[3′,4′:6,7]indolizino[1,2-b]quinoline-3,14(4H)-dione (Compound 117)

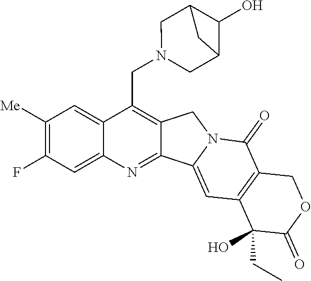 [SMILES: CC[C@@]1(O)C(=O)OCc2c1cc1n(c2=O)Cc2c-1nc1cc(F)c(C)cc1c2CN1CC2CC(C1)C2O]

The title compound was prepared according to General Procedure 1 starting from Compound 1.1 (10 mg) and 3-azabicyclo[3.1.1]heptan-6-ol. Preparative HPLC purification was accomplished as described in General Procedure 9, eluting with a 10 to 60% CH₃CN/H₂O+0.1% TFA gradient to give the title compound as an off-white solid (TFA salt, 1.3 mg, 11% yield).

LC/MS: Calc'd m/z=505.5 for C₂₈H₂₈FN305, found [M+H]⁺=506.6.

¹H NMR (300 MHz, 10% D₂O/CD3CN) 6 8.25 (d, J=7.9 Hz, 1H), 7.87 (d, J=10.6 Hz, 1H), 7.50 (s, 1H), 5.65-5.27 (m, 4H), 4.98 (s, 2H), 4.24 (s, 1H), 3.83-3.57 (m, 4H), 2.54 (s, 5H), 2.01-1.86 (m, 2H), 1.70 (s, 2H), 0.95 (t, J=7.3 Hz, 3H).

### 1.16: (S)-4-ethyl-8-fluoro-11-((3-fluoro-3-(hydroxymethyl)azetidin-1-yl)methyl)-4-hydroxy-9-methyl-1,12-dihydro-14H-pyrano[3′,4′:6,7]indolizino[1,2-b]quinoline-3,14(4H)-dione (Compound 118)

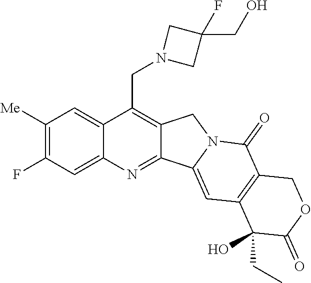 [SMILES: CC[C@@]1(O)C(=O)OCc2c1cc1n(c2=O)Cc2c-1nc1cc(F)c(C)cc1c2CN1CC(F)(CO)C1]

The title compound was prepared according to General Procedure 1 starting from Compound 1.1 (10 mg) and 3-fluoroazetidin-3-yl methanol. Preparative HPLC purification was accomplished as described in General Procedure 9, eluting with a 10 to 60% CH₃CN/H₂O+0.1% TFA gradient to give the title compound as an off-white solid (TFA salt, 1.4 mg, 12% yield).

LC/MS: Calc'd m/z=497.5 for C₂₆H₂₅F2N305, found [M+H]⁺=498.4.

¹H NMR (300 MHz, 10% D₂O/CD3CN) 6 8.24 (d, J=7.9 Hz, 1H), 7.85 (d, J=10.7 Hz, 1H), 7.50 (s, 1H), 5.57 (d, J=16.5 Hz, 1H), 5.48-5.28 (m, 3H), 4.98 (s, 2H), 4.44-4.14 (m, 4H), 3.78 (d, J=14.9 Hz, 2H), 2.01-1.86 (m, 2H), 0.95 (t, J=7.4 Hz, 3H).

### 1.17. (S)-4-ethyl-8-fluoro-4-hydroxy-11-((3-(hydroxymethyl)azetidin-1-yl)methyl)-9-methyl-1,12-dihydro-14H-pyrano[3′,4′:6,7]indolizino[1,2-b]quinoline-3,14(4H)-dione (Compound 119)

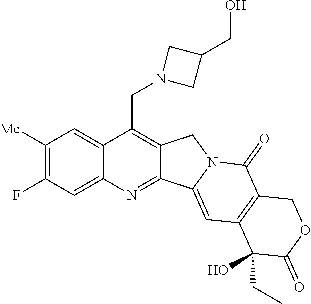 [SMILES: CC[C@@]1(O)C(=O)OCc2c1cc1n(c2=O)Cc2c-1nc1cc(F)c(C)cc1c2CN1CC(CO)C1]

The title compound was prepared according to General Procedure 1 starting from Compound 1.1 (10 mg) and azetidin-3-ylmethanol. Preparative HPLC purification was accomplished as described in General Procedure 9, eluting with a 10 to 60% CH₃CN/H₂O+0.1% TFA gradient to give the title compound as an off-white solid (TFA salt, 0.5 mg, 4.5% yield).

LC/MS: Calc'd m/z=479.5 for C₂₆H₂₆FN305, found [M+H]⁺=480.4.

¹H NMR (300 MHz, 10% D₂O/CD3CN) 6 8.23 (d, J=7.8 Hz, 1H), 7.90 (d, J=10.6 Hz, 1H), 7.53 (s, 1H), 5.58 (d, J=16.5 Hz, 1H), 5.50-5.28 (m, 3H), 5.01 (s, 2H), 4.31-4.17 (m, 2H), 4.15-4.00 (m, 2H), 3.62 (d, J=3.9 Hz, 2H), 2.58 (s, 3H), 2.01-1.86 (m, 2H), 0.96 (t, J=7.4 Hz, 3H).

### 1.18: (4S)-11-((4,4-difluoro-3-(hydroxymethyl)piperidin-1-yl)methyl)-4-ethyl-8-fluoro-4-hydroxy-9-methyl-1,12-dihydro-14H-pyrano[3′,4′:6,7]indolizino[1,2-b]quinoline-3,14(4H)-dione (Compound 120)

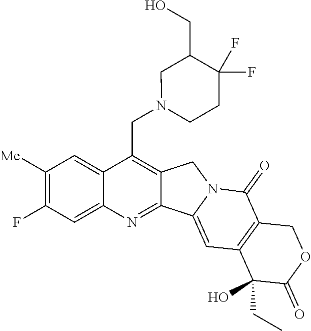 [SMILES: CC[C@@]1(O)C(=O)OCc2c1cc1n(c2=O)Cc2c-1nc1cc(F)c(C)cc1c2CN1CCC(F)(F)C(CO)C1]

The title compound was prepared according to General Procedure 1 starting from Compound 1.1 (10 mg) and 4,4-difluoropiperidin-3-yl methanol. Preparative HPLC purification was accomplished as described in General Procedure 9, eluting with a 10 to 60% CH₃CN/H₂O+0.1% TFA gradient to give the title compound as an off-white solid (TFA salt, 4 mg, 32% yield).

LC/MS: Calc'd m/z=543.5 for C₂₈H₂₈F3N305, found [M+H]⁺=544.4.

¹H NMR (300 MHz, 10% D₂O/CD3CN) 6 8.25 (d, J=8.0 Hz, 1H), 7.77 (dd, J=10.7, 1.4 Hz, 1H), 7.47 (s, 1H), 5.55 (d, J=16.5 Hz, 1H), 5.42-5.25 (m, 3H), 4.66 (d, J=3.2 Hz, 2H), 3.90-3.77 (m, 1H), 3.71-3.45 (m, 4H), 2.24 (q, J=11.8, 9.2 Hz, 2H), 2.01-1.86 (m, 2H), 0.94 (t, J=7.4 Hz, 3H).

### 1.19: (S)-4-ethyl-8-fluoro-4-hydroxy-11-((1-(hydroxymethyl)-7-azabicyclo[2.2.1]heptan-7-yl)methyl)-9-methyl-1,12-dihydro-14H-pyrano[3′,4′:6,7]indolizino[1,2-b]quinoline-3,14(4H)-dione (Compound 121)

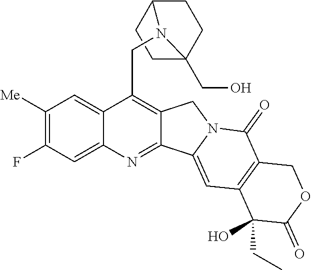 [SMILES: CC[C@@]1(O)C(=O)OCc2c1cc1n(c2=O)Cc2c-1nc1cc(F)c(C)cc1c2CN1C2CCC1(CO)CC2]

The title compound was prepared according to General Procedure 1 starting from Compound 1.1 (10 mg) and 7-azabicyclo[2.2.1]heptan-1-ylmethanol. Preparative HPLC purification was accomplished as described in General Procedure 9, eluting with a 10 to 60% CH₃CN/H₂O+0.1% TFA gradient to give the title compound as an off-white solid (TFA salt, 0.8 mg, 6.6% yield).

LC/MS: Calc'd m/z=519.6 for C₂₉H₃₀FN305, found [M+H]⁺=520.4.

¹H NMR (300 MHz, 10% D₂O/CD3CN) 6 8.22 (s, 1H), 7.92 (d, J=10.7 Hz, 1H), 7.54 (s, 1H), 5.59 (dd, J=17.6, 7.6 Hz, 2H), 5.33 (t, J=17.4 Hz, 2H), 4.98-4.81 (m, 1H), 4.67-4.44 (m, 2H), 4.28-3.93 (m, 4H), 2.73 (s, 2H), 2.34-2.03 (m, 4H), 1.91 (d, J=14.0 Hz, 5H), 0.96 (t, J=7.4 Hz, 3H).

### 1.20: (S)-N-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)methanesulfonamide (Compound 122)

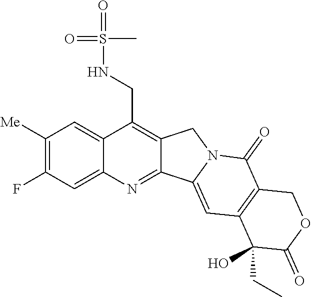 [SMILES: CC[C@@]1(O)C(=O)OCc2c1cc1n(c2=O)Cc2c-1nc1cc(F)c(C)cc1c2CNS(C)(=O)=O]

The title compound was prepared according to General Procedure 3 starting from Compound 1.2 (10 mg) and methane sulfonyl chloride. Preparative HPLC purification was accomplished as described in General Procedure 9, eluting with a 10 to 50% CH₃CN/H₂O+0.1% TFA gradient to give the title compound as an off-white solid (0.8 mg, 7% yield).

LC/MS: Calc'd m/z=487.1 for C₂₃H₂₂FN306S, found [M+H]⁺=488.2.

¹H NMR (300 MHz, MeOD) δ 8.33 (d, J=8.1 Hz, 1H), 7.83 (d, J=10.8 Hz, 1H), 7.68 (s, 1H), 5.62 (d, J=16.3 Hz, 1H), 5.52 (s, 2H), 5.42 (d, J=16.4 Hz, 1H), 4.87 (s, 2H), 3.06 (s, 3H), 2.59 (s, 3H), 2.06-1.93 (m, 2H), 1.03 (t, J=7.4 Hz, 3H).

### 1.21: (S)—N-((4-ethyl-8-fluoro-4-hydroxy-9-methyl-3,14-dioxo-3,4,12,14-tetrahydro-1H-pyrano[3′,4′ 6,7]indolizino[1,2-b]quinolin-11-yl)methyl)-1-(4-nitrophenyl) methanesulfonamide (Compound 124)

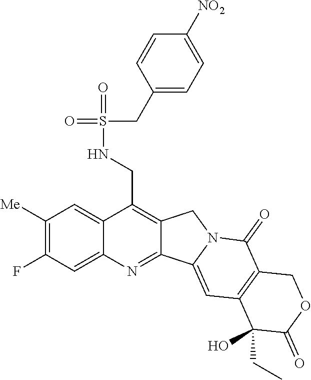 [SMILES: CC[C@@]1(O)C(=O)OCc2c1cc1n(c2=O)Cc2c-1nc1cc(F)c(C)cc1c2CNS(=O)(=O)Cc1ccc([N+](=O)[O-])cc1]

The title compound was prepared according to General Procedure 3 starting from Compound 1.2 (20 mg) and (4-nitrophenyl)methanesulfonyl chloride. Preparative HPLC purification was accomplished as described in General Procedure 9, eluting with a 10 to 50% CH₃CN/H₂O+0.1% TFA gradient to give the title compound as an off-white solid (5.0 mg, 17% yield).

LC/MS: Calc'd m/z=608.1 for C₂₉H₂₅FN408S, found [M+H]⁺=609.2.

¹H NMR (300 MHz, CDCl₃) δ 8.02-7.92 (m, 3H), 7.74 (d, J=10.5 Hz, 1H), 7.65 (s, 1H), 7.33 (d, J=8.6 Hz, 2H), 5.66 (d, J=16.8 Hz, 1H), 5.28 (d, J=16.5 Hz, 1H), 5.14 (d, J=5.4 Hz, 2H), 4.67 (s, 2H), 4.28 (d, J=6.3 Hz, 2H), 3.39 (s, 3H), 2.03-1.83 (m, 2H), 1.04 (t, J=7.4 Hz, 3H).

### 1.22: (S)—N-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)benzenesulfonamide (Compound 125)

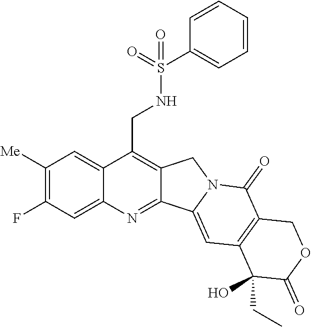 [SMILES: CC[C@@]1(O)C(=O)OCc2c1cc1n(c2=O)Cc2c-1nc1cc(F)c(C)cc1c2CNS(=O)(=O)c1ccccc1]

The title compound was prepared according to General Procedure 3 starting from Compound 1.2 (10 mg) and benzenesulfonyl chloride. Preparative HPLC purification was accomplished as described in General Procedure 9, eluting with a 10 to 50% CH₃CN/H₂O+0.1% TFA gradient to give the title compound as an off-white solid (9.8 mg, 73% yield).

LC/MS: Calc'd m/z=549.6 for C₂₈H₂₄FN306S, found [M+H]⁺=550.6.

¹H NMR (300 MHz, DMSO-d6) δ 8.60 (t, J=6.2 Hz, 1H), 8.17 (d, J=8.1 Hz, 1H), 7.83 (d, J=10.8 Hz, 1H), 7.71 (dd, J=7.1, 1.7 Hz, 2H), 7.66-7.48 (m, 2H), 7.46 (dd, J=8.3, 6.8 Hz, 2H), 7.40-7.27 (m, 2H), 7.18 (s, 1H), 7.01 (s, 1H), 5.45 (s, 2H), 5.33 (s, 2H), 4.63 (d, J=6.2 Hz, 2H), 2.48 (s, 3H), 1.98-1.76 (m, 2H), 0.89 (t, J=7.3 Hz, 3H).

### 1.23: (S)-N-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-4-nitrobenzenesulfonamide (Compound 1.23)

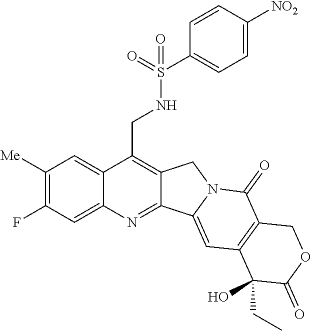 [SMILES: CC[C@@]1(O)C(=O)OCc2c1cc1n(c2=O)Cc2c-1nc1cc(F)c(C)cc1c2CNS(=O)(=O)c1ccc([N+](=O)[O-])cc1]

The title compound was prepared according to General Procedure 3 starting from Compound 1.2 (75 mg) and 4-nitrobenzenesulfonyl chloride. Purification of the title compound was accomplished as described in General Procedure 9, using a 12 g C₁₈ column and eluting with a 5 to 75% CH₃CN/H₂O+0.1% TFA gradient to give the title compound as an off-white solid (37.8 mg, 47% yield).

LC/MS: Calc'd m/z=594.6 for C₂₈H₂₃FN408S, found [M+H]⁺=595.2.

### 1.24: (S)-4-amino-N-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)benzenesulfonamide (Compound 127)

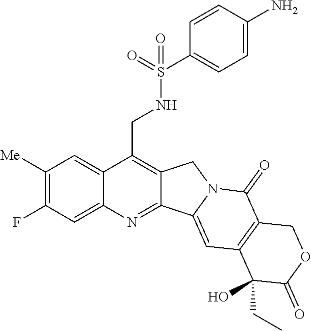 [SMILES: CC[C@@]1(O)C(=O)OCc2c1cc1n(c2=O)Cc2c-1nc1cc(F)c(C)cc1c2CNS(=O)(=O)c1ccc(N)cc1]

To a solution of Compound 1.23 (37.8 mg, 0.064 mmol) in methanol (6.4 mL) was added platinum 1% vanadium 2% on carbon (75 mg). The flask was purged with H₂ then stirred at room temperature under an H₂ atmosphere for 45 min. The mixture was filtered through a pad of celite, washed with DMF, and the filtrate evaporated to give the title compound as a pale yellow solid (30 mg, 84% yield).

LC/MS: Calc'd m/z=564.6 for C₂₈H₂₄FN406S, found [M+H]⁺=565.2.

¹H NMR (300 MHz, DMSO-d6) δ 8.13 (d, J=8.2 Hz, 1H), 8.02 (t, J=6.2 Hz, 1H), 7.88 (d, J=10.8 Hz, 1H), 7.48-7.35 (m, 2H), 7.31 (d, J=8.4 Hz, 1H), 6.63-6.45 (m, 2H), 5.45 (s, 2H), 5.36 (s, 2H), 4.50 (d, J=6.3 Hz, 2H), 1.98-1.75 (m, 2H), 0.89 (t, J=7.3 Hz, 3H).

### 1.25: (S)-N-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-2-hydroxyethane-1-sulfonamide (Compound 129)

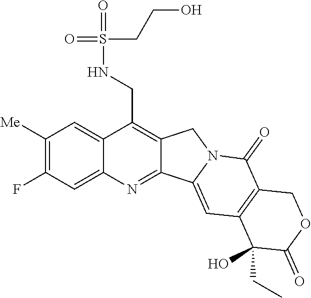 [SMILES: CC[C@@]1(O)C(=O)OCc2c1cc1n(c2=O)Cc2c-1nc1cc(F)c(C)cc1c2CNS(=O)(=O)CCO]

The title compound was prepared according to General Procedure 3 starting from Compound 1.2 (20 mg) and 2-hydroxyethanesulfonyl chloride. Preparative HPLC purification was accomplished as described in General Procedure 9, eluting with a 25 to 50% CH₃CN/H₂O+0.1% TFA gradient to give the title compound as an off-white solid (1.3 mg, 13% yield).

LC/MS: Calc'd m/z=517.1 for C₂₄H₂₄FN307S, found [M+H]⁺=518.2.

¹H NMR (300 MHz, DMSO-d6) δ 8.30 (d, J=8.4 Hz, 1H), 7.91 (d, J=10.9 Hz, 1H), 7.84 (t, J=6.3 Hz, 1H), 7.33 (s, 1H), 5.50-5.33 (m, 4H), 5.07 (t, J=5.4 Hz, 1H), 4.78 (d, J=6.0 Hz, 2H), 4.07 (s, 3H), 3.80 (dt, J=6.3 Hz, J=5.8 Hz, 2H), 1.86 (m, 2H), 0.87 (d, J=7.3 Hz, 3H).

### 1.26: (S)-N-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)methanesulfamide (Compound 131)

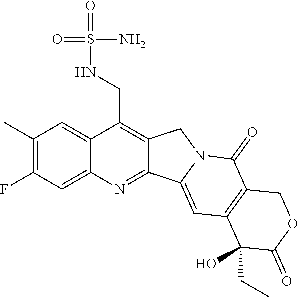 [SMILES: CC[C@@]1(O)C(=O)OCc2c1cc1n(c2=O)Cc2c-1nc1cc(F)c(C)cc1c2CNS(N)(=O)=O]

To a solution of chlorosulfonyl isocyanate (3 μL) in dichloromethane (1 mL) was added tert-butanol (3 μL). This solution was stirred for 1 h, then Compound 1.2 (13 mg) dissolved in dichloromethane (1 mL) was added followed by triethylamine (13 μL). The reaction was stirred for 1 hr then concentrated to dryness. Preparative HPLC purification of the intermediate Boc compound was accomplished as described in General Procedure 9, eluting with a 10 to 50% CH₃CN/H₂O+0.1% TFA gradient. To the purified solid in dichloromethane (1 mL) was added trifluoroacetic acid (200 μL). The reaction was stirred for 16 h then concentrated to dryness to provide the title compound as an off-white solid (7.5 mg, 48% yield).

LC/MS: Calc'd m/z=488.1 for C₂₂H₂₁FN406S, found [M+H]⁺=489.0.

¹H NMR (300 MHz, MeOD) δ 8.25 (d, J=8.1 Hz, 1H), 7.73 (d, J=10.7 Hz, 1H), 7.62 (s, 1H), 5.59 (d, J=16.4 Hz, 1H), 5.45 (s, 2H), 5.39 (d, J=16.4 Hz, 1H), 4.81 (s, 2H), 2.55 (d, J=1.7 Hz, 3H), 2.07-1.89 (m, 2H), 1.03 (t, J=7.4 Hz, 3H).

### 1.27: 4-nitrophenyl-(S)-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)carbamate (Compound 1.27)

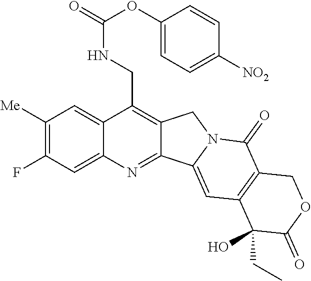 [SMILES: CC[C@@]1(O)C(=O)OCc2c1cc1n(c2=O)Cc2c-1nc1cc(F)c(C)cc1c2CNC(=O)Oc1ccc([N+](=O)[O-])cc1]

The title PNP-carbamate intermediate compound was prepared according to the first step of General Procedure 4 starting from Compound 1.2 (24 mg). Purification was accomplished as described in General Procedure 9, using a 12 g column C₁₈ column and eluting with a 10 to 50% CH₃CN/H₂O+0.1% TFA gradient to give the title compound as an off-white solid (14 mg, 53% yield).

LC/MS: Calc'd m/z=574.2 for C₂₉H₂₃FN408S, found [M+H]⁺=575.2

### 1.28: (S)-1-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-3-methylurea (Compound 132)

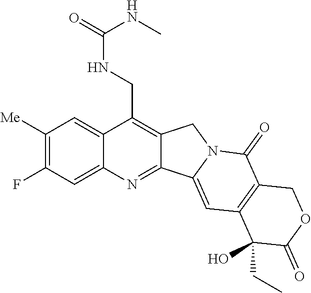 [SMILES: CC[C@@]1(O)C(=O)OCc2c1cc1n(c2=O)Cc2c-1nc1cc(F)c(C)cc1c2CNC(=O)NC]

The title compound was prepared according to General Procedure 4 starting from Compound 1.2 (25 mg) and aqueous methyl amine (500 μL, 40 wt. % in water) as the primary amine. In this instance, the intermediate PNP carbamate was used crude. Preparative HPLC purification was accomplished as described in General Procedure 9, eluting with a 10 to 50% CH₃CN/H₂O+0.1% TFA gradient to give the title compound as an off-white solid (8.9 mg, 31% yield).

LC/MS: Calc'd m/z=466.2 for C₂₄H₂₃FN405, found [M+H]⁺=467.2.

¹H NMR (300 MHz, MeOD) δ 8.26 (d, J=8.2 Hz, 1H), 7.79 (d, J=10.7 Hz, 1H), 7.66 (s, 1H), 5.61 (d, J=16.3 Hz, 1H), 5.48 (s, 2H), 5.41 (d, J=16.4 Hz, 1H), 4.97 (s, 2H), 2.73 (s, 3H), 2.57 (s, 3H), 2.08-1.93 (m, 2H), 1.03 (t, J=7.4 Hz, 3H).

### 1.29: (S)-1-(4-aminobenzyl)-3-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)urea (Compound 134)

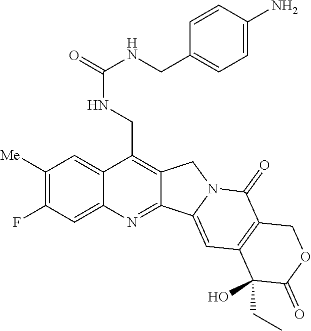 [SMILES: CC[C@@]1(O)C(=O)OCc2c1cc1n(c2=O)Cc2c-1nc1cc(F)c(C)cc1c2CNC(=O)NCc1ccc(N)cc1]

The title compound was prepared according to the second step of General Procedure 4 using Compound 1.27 (4 mg) as the PNP-carbamate and 4-(aminomethyl)aniline as the primary amine. Preparative HPLC purification was accomplished as described in General Procedure 9, eluting with a 20 to 50% CH₃CN/H₂O+0.1% TFA gradient to give the title compound as an off-white solid (0.6 mg, 12% yield).

LC/MS: Calc'd m/z=557.2 for C₃₀H28FN505, found [M+H]⁺=558.4.

¹H NMR (300 MHz, MeOD) δ 8.25 (d, J=8.1 Hz, 1H), 7.80 (d, J=10.8 Hz, 1H), 7.67 (s, 1H), 7.43 (d, J=8.2 Hz, 2H), 7.24 (d, J=8.3 Hz, 2H), 5.63 (d, J=16.4 Hz, 1H), 5.48 (s, 2H), 5.43 (d, J=16.4 Hz, 1H), 5.01 (s, 2H), 4.37 (s, 2H), 2.56 (d, J=1.7 Hz, 3H), 2.05-1.94 (m, 2H), 1.03 (t, J=7.3 Hz, 3H).

### 1.30: (S)-1-((4-ethyl-8-fluoro-4-hydroxy-9-methyl-3,14-dioxo-3,4,12,14-tetrahydro-1H-pyrano[3′,4′ 6,7]indolizino[1,2-b]quinolin-11-yl)methyl)-3-(2-hydroxyethyl)urea (Compound 136)

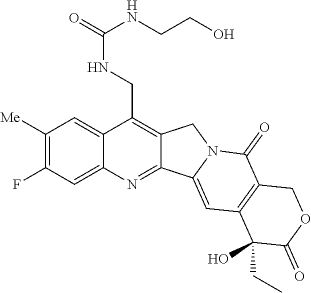 [SMILES: CC[C@@]1(O)C(=O)OCc2c1cc1n(c2=O)Cc2c-1nc1cc(F)c(C)cc1c2CNC(=O)NCCO]

The title compound was prepared according to the second step of General Procedure 4 using Compound 1.27 (4 mg) as the PNP-carbamate and hydroxyethylamine as the primary amine. Preparative HPLC purification was accomplished as described in General Procedure 9, eluting with a 10 to 50% CH₃CN/H₂O+0.1% TFA gradient to give the title compound as an off-white solid (2.4 mg, 66% yield).

LC/MS: Calc'd m/z=496.2 for C₂₅H₂₅FN406, found [M+H]⁺=497.2.

¹H NMR (300 MHz, MeOD) δ 8.08 (d, J=8.0 Hz, 1H), 7.74 (d, J=10.5 Hz, 1H), 7.68 (s, 1H), 5.64 (d, J=16.4 Hz, 1H), 5.41 (s, 2H), 5.31 (d, J=16.4 Hz, 1H), 4.96 (s, 2H), 3.63 (t, J=5.2 Hz, 2H), 3.29 (t, J=5.3 Hz, 2H), 2.54 (s, 3H), 1.98-1.87 (m, 2H), 1.01 (t, J=7.4 Hz, 3H).

### 1.31: Methyl-(S)-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)carbamate (Compound 138)

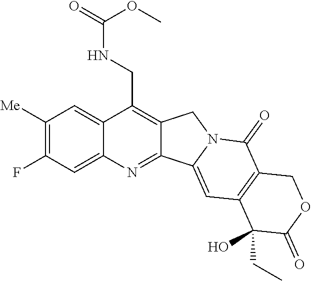 [SMILES: CC[C@@]1(O)C(=O)OCc2c1cc1n(c2=O)Cc2c-1nc1cc(F)c(C)cc1c2CNC(=O)OC]

The title compound was prepared according to General Procedure 5 starting from Compound 1.2 (50 mg) and reacting methanol with the intermediate PNP-carbamate. Preparative HPLC purification was accomplished as described in General Procedure 9, eluting with a 20 to 50% CH₃CN/H₂O+0.1% TFA gradient to give the title compound as an off-white solid (3.5 mg, 6% yield).

LC/MS: Calc'd m/z=467.2 for C₂₄H₂₂FN306, found [M+H]⁺=468.2.

¹H NMR (300 MHz, MeOD) δ 8.17 (d, J=8.2 Hz, 1H), 7.77 (d, J=10.5 Hz, 1H), 7.69 (s, 1H), 5.65 (d, J=16.5 Hz, 1H), 5.48 (s, 2H), 5.33 (d, J=16.4 Hz, 1H), 4.86 (d, J=5.6 Hz, 2H), 3.65 (s, 3H), 2.56 (s, 3H), 2.02-1.89 (m, 2H), 1.02 (t, J=7.4 Hz, 3H).

### 1.32: 2-hydroxyethyl (S)-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)carbamate (Compound 139)

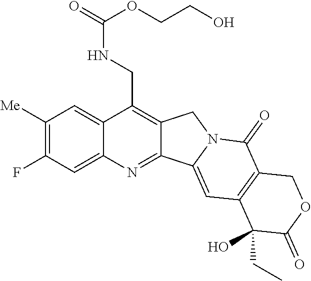 [SMILES: CC[C@@]1(O)C(=O)OCc2c1cc1n(c2=O)Cc2c-1nc1cc(F)c(C)cc1c2CNC(=O)OCCO]

The title compound was prepared according to General Procedure 5 starting from Compound 1.2 (18 mg) and reacting 1,2-ethanediol with the intermediate PNP-carbamate. Preparative HPLC purification was accomplished as described in General Procedure 9, eluting with a 10 to 60% CH₃CN/H₂O+0.1% TFA gradient to give the title compound as an off-white solid (4.2 mg, 19% yield).

LC/MS: Calc'd m/z=497.2 for C₂₅H₂₄FN307, found [M+H]⁺=498.2.

¹H NMR (300 MHz, DMSO) δ 8.23 (d, J=8.2 Hz, 1H), 7.78 (d, J=10.7 Hz, 1H), 7.40 (s, 1H), 5.47 (d, J=16.5 Hz, 1H), 5.42 (s, 2H), 5.34 (d, J=16.4 Hz, 1H), 4.77 (s, 2H), 3.99 (t, J=4.9 Hz, 2H), 3.64-3.38 (m, 2H), 2.48 (s, 3H), 2.02-1.67 (m, 2H), 0.89 (t, J=7.3 Hz, 3H).

### Example 2: Preparation of Camptothecin Analogues Having Methoxy at the C10 Position

### 2.1: 1-(2-anino-4-fluoro-5-methoxyphenyl)-2-chloroethan-1-one (Compound 2.1)

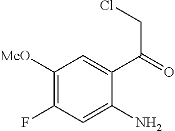 [SMILES: COc1cc(C(=O)CCl)c(N)cc1F]

A solution of 3-fluoro-4-methoxyaniline (10 g, 71 mmol) in DCM (100 mL) was cooled to 0° C. To this solution was first added a 1 M BCl₃ in DCM (71 mL, 71 mmol), followed by a 1 M chloro(diethyl)alumane in DCM (71 mL, 71 mmol), then finally 2-chloroacetonitrile (6.4 g, 85 mmol). The solution was heated at reflux for 3 h, cooled to room temperature, and quenched by the addition of an aqueous 2 M HCl solution. The resulting heterogenous mixture was heated to reflux for 1 h, cooled to room temperature, then the pH was adjusted to ˜12 with Na2CO₃. The layers were separated, and the aqueous layer extracted with DCM (3×100 mL). The combined organic layers were dried over Na₂SO₄, concentrated, and flash purified as described in General Procedure 9, eluting with 0 to 20% EtOAc/Hexanes to give the title compound (6 g, 28 mmol, 39% yield).

LC/MS: Calc'd m/z=217.1 for C₉H₉ClFNO₂, found [M+H]⁺=218.1.

¹H NMR (400 MHz, CDCl₃) δ 7.19 (d, J=9.2 Hz, 1H), 6.44 (d, J=12.8 Hz, 1H), 4.59 (s, 2H), 3.86 (s, 3H)

### 2.2: (S)-11-(chloromethyl)-4-ethyl-8-fluoro-4-hydroxy-9-methoxy-1,12-dihydro-14H-pyrano[3′,4′ 6,7]indolizino[1,2-b]quinoline-3,14(4H)-dione (Compound 2.2)

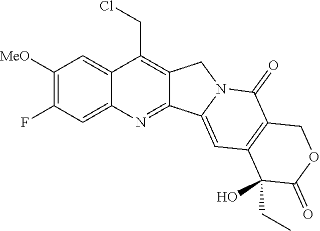 [SMILES: CC[C@@]1(O)C(=O)OCc2c1cc1n(c2=O)Cc2c-1nc1cc(F)c(OC)cc1c2CCl]

To a solution of Compound 2.1 (1.65 g, 7.6 mmol) and (S)-4-ethyl-4-hydroxy-7,8-dihydro-1H-pyrano[3,4-f]indolizine-3,6,10(4H)-trione (2 g, 7.6 mmol) in toluene (200 mL) was added toluene-4-sulfonic acid (157 mg, 0.9 mmol). This solution was heated at 140° C. for 3 h then cooled to room temperature. The product as yellow precipitate was collected by filtration to give the title compound (1.27 g, 2.85 mmol, 37.5% yield).

LC/MS: Calc'd m/z=445.2 for C₂₂H₁₈C₁FN205, found [M+H]⁺=445.1.

¹H NMR (400 MHz, DMSO-d6) δ 7.99 (d, J=12.0 Hz, 1H) 7.80 (d, J=9.2 Hz, 1H) 7.27 (s, 1H), 6.50 (s, 1H), 5.45 (s, 2H), 5.41 (s, 2H), 5.33 (s, 2H) 4.08 (s, 3H), 1.87-1.83 (m, 2H), 0.87 (t, J=7.2 Hz, 3H)

### 2.3: (S)-4-ethyl-8-fluoro-4-hydroxy-9-methoxy-11-(morpholinomethyl)-1,12-dihydro-14H-pyrano[3′,4′:6,7]indolizino[1,2-b]quinoline-3,14(4H)-dione (Compound 101)

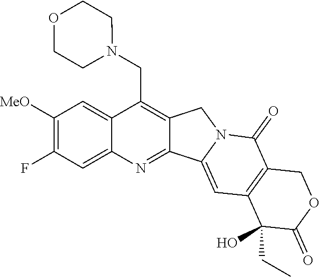 [SMILES: CC[C@@]1(O)C(=O)OCc2c1cc1n(c2=O)Cc2c-1nc1cc(F)c(OC)cc1c2CN1CCOCC1]

The title compound was prepared according to General Procedure 1 starting from Compound 2.2 (10 mg) and morpholine. Preparative HPLC purification was accomplished as described in General Procedure 9, eluting with a 20 to 60% CH₃CN/H₂O+0.1% TFA gradient to give the title compound as an off-white solid (5.6 mg, 41% yield).

LC/MS: Calc'd m/z=495.2 for C₂₆H₂₆FN306, found [M+H]⁺=496.4.

¹H NMR (300 MHz, MeOD) δ 7.84-7.70 (m, 2H), 7.59 (s, 1H), 5.62 (d, J=16.3 Hz, 1H), 5.45-5.36 (m, 3H), 4.29 (s, 2H), 4.12 (s, 3H), 3.58-3.48 (m, 2H), 3.28-3.09 (m, 2H), 2.75-2.61 (m, 2H), 2.05-1.91 (m, 2H), 1.02 (t, J=7.4 Hz, 3H).

### 2.4: (S)-4-ethyl-8-fluoro-4-hydroxy-9-methoxy-11-((4-(phenylsulfonyl)piperazin-1-yl)methyl)-1,12-dihydro-14H-pyrano[3′,4′: 6,7]indolizino[1,2-b]quinoline-3,14(4H)-dione (Compound 103)

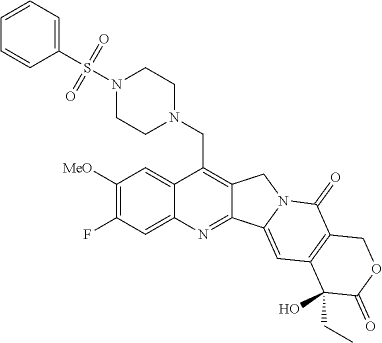 [SMILES: CC[C@@]1(O)C(=O)OCc2c1cc1n(c2=O)Cc2c-1nc1cc(F)c(OC)cc1c2CN1CCN(S(=O)(=O)c2ccccc2)CC1]

The title compound was prepared according to General Procedure 1 starting from Compound 2.2 (10 mg) and 1-(phenylsulfonyl)piperazine. Preparative HPLC purification was accomplished as described in General Procedure 9, eluting with a 20 to 60% CH₃CN/H₂O+0.1% TFA gradient to give the title compound as an off-white solid (2.5 mg, 14% yield).

LC/MS: Calc'd m/z=634.2 for C₃₂H₃₁FN407S, found [M+H]⁺=635.4.

### 2.5: (S)-11-((4-((4-aminophenyl)sulfonyl)piperazin-1-yl)methyl)-4-ethyl-8-fluoro-4-hydroxy-9-methoxy-1,12-dihydro-14H-pyrano[3′,4′:6,7]indolizino[1,2-b]quinoline-3,14(4H)-dione (Compound 105)

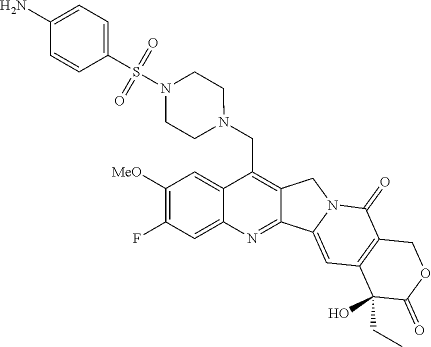 [SMILES: CC[C@@]1(O)C(=O)OCc2c1cc1n(c2=O)Cc2c-1nc1cc(F)c(OC)cc1c2CN1CCN(S(=O)(=O)c2ccc(N)cc2)CC1]

The title compound was prepared according to General Procedure 1 starting from Compound 2.2 (10 mg) and 4-(piperazin-1-ylsulfonyl)aniline. Preparative HPLC purification was accomplished as described in General Procedure 9, eluting with a 20 to 60% CH₃CN/H₂O+0.1% TFA gradient to give the title compound as an off-white solid (4.0 mg, 23% yield).

LC/MS: Calc'd m/z=649.2 for C₃₂H₃₂FN₅O₇S, found [M+H]+=650.4.

¹H NMR (300 MHz, DMSO) δ 8.08 (s, 2H), 7.90-7.67 (m, 2H), 7.35 (s, 1H), 7.32-7.26 (m, 2H), 6.67-6.57 (m, 2H), 5.46 (d, J=16.5 Hz, 1H), 5.33-5.22 (m, 3H), 3.92 (s, 3H), 3.02-2.72 (m, 4H), 2.75-2.58 (m, 4H), 1.97-1.70 (m, 2H), 0.90 (t, J=7.3 Hz, 3H).

### 2.6: (S)-4-ethyl-8-fluoro-4-hydroxy-9-methoxy-11-((4-methylpiperazin-1-yl)methyl)-1,12-dihydro-14H-pyrano[3′,4′:6,7]indolizino[1,2-b]quinoline-3,14(4H)-dione (Compound 107)

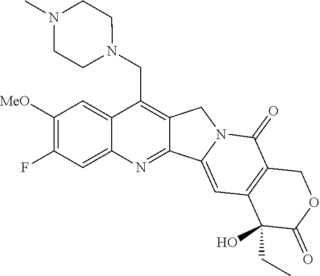 [SMILES: CC[C@@]1(O)C(=O)OCc2c1cc1n(c2=O)Cc2c-1nc1cc(F)c(OC)cc1c2CN1CCN(C)CC1]

The title compound was prepared according to General Procedure 1 starting from Compound 2.2 (10 mg) and N-methylpiperazine. Preparative HPLC purification was accomplished as described in General Procedure 9, eluting with a 20 to 60% CH₃CN/H₂O+0.1% TFA gradient to give the title compound as an off-white solid (2.1 mg, 19% yield).

LC/MS: Calc'd m/z=508.2 for C₂₇H₂₉FN₄O₅, found [M+H]⁺=509.4.

### 2.7: (S)-11-((4-(4-aminophenyl)piperazin-1-yl)methyl)-4-ethyl-8-fluoro-4-hydroxy-9-methoxy-1,12-dihydro-14H-pyrano[3′,4′: 6,7]indolizino[1,2-b]quinoline-3,14(4H)-dione (Compound 109)

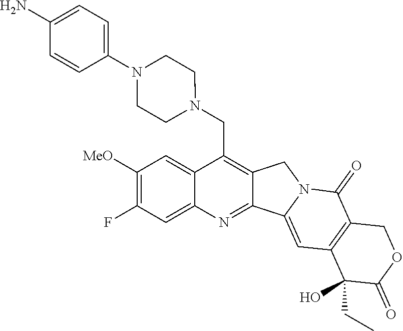 [SMILES: CC[C@@]1(O)C(=O)OCc2c1cc1n(c2=O)Cc2c-1nc1cc(F)c(OC)cc1c2CN1CCN(c2ccc(N)cc2)CC1]

The title compound was prepared according to General Procedure 1 starting from Compound 2.2 (10 mg) and 4-(piperazin-1-yl)aniline. Preparative HPLC purification was accomplished as described in General Procedure 9, eluting with a 20 to 60% CH₃CN/H₂O+0.1% TFA gradient to give the title compound as an off-white solid (3.2 mg, 20% yield).

LC/MS: Calc'd m/z=585.2 for C₃₂H₃₂FN₅O₅, found [M+H]⁺=586.4.

¹H NMR (300 MHz, MeOD) δ 7.83-7.74 (m, 2H), 7.62 (s, 1H), 7.06 (d, J=8.9 Hz, 2H), 6.98 (d, J=8.9 Hz, 2H), 5.65 (d, J=16.4 Hz, 1H), 5.36 (s, 2H), 5.27 (d, J=16.4 Hz, 1H), 4.13 (s, 2H), 4.06 (s, 3H), 3.26 (br s, 4H), 2.79 (br s, 4H), 1.97-1.83 (m, 2H), 1.00 (t, J=7.4 Hz, 3H).

### 2.8: (S)-11-(aminomethyl)-4-ethyl-8-fluoro-4-hydroxy-9-methoxy-1,12-dihydro-14H-pyrano[3′,4′:6,7]indolizino[1,2-b]quinoline-3,14(4H)-dione (Compound 2.8)

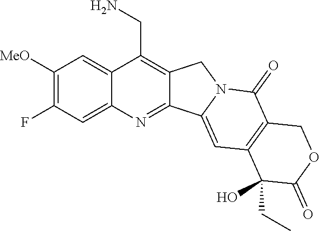 [SMILES: CC[C@@]1(O)C(=O)OCc2c1cc1n(c2=O)Cc2c-1nc1cc(F)c(OC)cc1c2CN]

To a solution of Compound 2.2 (250 mg, 0.56 mmol) in ethanol (7 mL) was added hexamethylenetetramine (236 mg, 1.7 mmol) followed by iPr₂NEt (100 μL, 0.56 mmol). This solution was heated at reflux for 5 h, cooled to room temperature and quenched with 12 M aqueous HCl (60 μL). This solution was concentrated to 1₂ volume and 1 M aqueous HCl (1.5 mL) was added, stirred for 5 min, then concentrated to give a brown residue. Purification was accomplished as described in General Procedure 9, using a 12 g C18 flash column and eluting with a 5 to 40% CH₃CN/H₂O+0.1% TFA gradient to give the title compound as pale yellow solid (179 mg, 75% yield).

LC/MS: Calc'd m/z=425.4 for C₂₂H₂₀FN₃O₅, found [M+H]⁺=426.2

### 2.9: (S)—N-((4-ethyl-8-fluoro-4-hydroxy-9-methoxy-3,14-dioxo-3,4,12,14-tetrahydro-1H-pyrano[3′, 4′: 6,7]indolizino[1,2-b]quinolin-11-yl)methyl)methanesulfonamide (Compound 123)

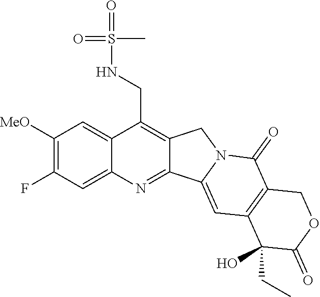 [SMILES: CC[C@@]1(O)C(=O)OCc2c1cc1n(c2=O)Cc2c-1nc1cc(F)c(OC)cc1c2CNS(C)(=O)=O]

The title compound was prepared according to General Procedure 3 starting from Compound 2.8 (10 mg) and methanesulfonyl chloride. Preparative HPLC purification was accomplished as described in General Procedure 9, eluting with a 5 to 65% CH₃CN/H₂O+0.1% TFA gradient to give the title compound as an off-white solid (8.5 mg, 91% yield).

LC/MS: Calc'd m/z=503.1 for C₂₃H₂₂FN₃O₇S, found [M+H]⁺=504.2.

¹H NMR (300 MHz, DMSO-d6) δ 7.98 (d, J=12.1 Hz, 1H), 7.89 (t, J=6.4 Hz, 1H), 7.80 (d, J=9.1 Hz, 1H), 7.28 (s, 1H), 5.42 (s, 2H), 5.39 (s, 2H), 4.77 (d, J=6.4 Hz, 2H), 4.06 (s, 3H), 3.06 (s, 3H), 1.95-1.73 (m, 2H), 0.88 (d, J=7.3 Hz, 3H).

### 2.10: (S)—N-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)benzenesulfonamide (Compound 126)

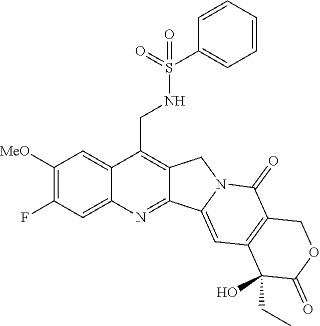 [SMILES: CC[C@@]1(O)C(=O)OCc2c1cc1n(c2=O)Cc2c-1nc1cc(F)c(OC)cc1c2CNS(=O)(=O)c1ccccc1]

The title compound was prepared according to General Procedure 3 starting from Compound 2.8 (7.5 mg) and benzenesulfonyl chloride. Preparative HPLC purification was accomplished as described in General Procedure 9, eluting with a 5 to 70% CH₃CN/H₂O+0.1% TFA gradient to give the title compound as an off-white solid (4.6 mg, 46% yield).

LC/MS: Calc'd m/z=565.6 for C₂₈H₂₄FN₃O₇S, found [M+H]⁺=566.2.

¹H NMR (300 MHz, DMSO-d6) δ 8.59 (t, J=6.3 Hz, 1H), 7.94 (d, J=12.2 Hz, 1H), 7.82-7.68 (m, 2H), 7.62-7.46 (m, 1H), 7.51-7.40 (m, 1H), 7.28 (d, J=8.3 Hz, 1H), 6.52 (s, 1H), 5.44 (s, 1H), 5.36 (s, 1H), 4.64 (d, J=6.3 Hz, 1H), 4.09 (s, 2H), 1.95-1.81 (m, 1H), 0.89 (t, J=7.3 Hz, 2H).

### 2.11: (S)—N-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-4-nitrobenzenesulfonamide (Compound 2.11)

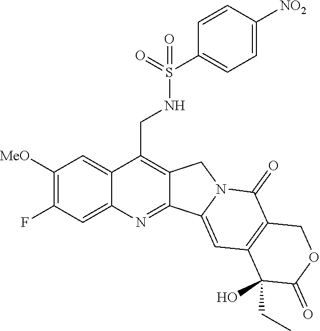 [SMILES: CC[C@@]1(O)C(=O)OCc2c1cc1n(c2=O)Cc2c-1nc1cc(F)c(OC)cc1c2CNS(=O)(=O)c1ccc([N+](=O)[O-])cc1]

The title compound was prepared according to General Procedure 3 starting from Compound 2.8 (12 mg) and 4-nitrobenzenesulfonyl chloride. Purification was accomplished as described in General Procedure 9 using a 12 g C18 flash column and eluting with a 5 to 75% CH₃CN/H₂O+0.1% TFA gradient to give the title compound as pale yellow solid (9.7 mg, 71% yield).

LC/MS: Calc'd m/z=610.6 for C₂₈H₂₃FN₄O₉S, found [M+H]⁺=611.5.

### 2.12: (S)-4-amino-N-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)benzenesulfonamide (Compound 128)

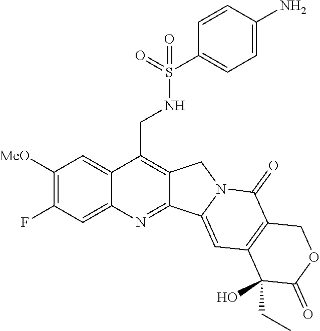 [SMILES: CC[C@@]1(O)C(=O)OCc2c1cc1n(c2=O)Cc2c-1nc1cc(F)c(OC)cc1c2CNS(=O)(=O)c1ccc(N)cc1]

To a solution of Compound 2.11 (9.7 mg, 0.016 mmol) in methanol (1.6 mL) was added platinum 1% vanadium 2% on carbon (15 mg). The flask was purged with H₂ then stirred at room temperature under an H₂ atmosphere for 45 min. The mixture was filtered through a pad of celite, washed with DMF, then the filtrate was evaporated to give the title compound as a pale yellow solid (1.5 mg, 16% yield).

LC/MS: Calc'd m/z=580.6 for C₂₈H₂₅FN₄O₇S, found [M+H]⁺=581.4.

¹H NMR (300 MHz, MeOD) δ 7.77 (d, J=11.0 Hz, 1H), 7.58 (s, 1H), 7.48 (d, J=8.6 Hz, 1H), 6.61 (d, J=8.6 Hz, 1H), 5.59 (d, J=16.3 Hz, 1H), 5.39 (d, J=16.4 Hz, 1H), 5.30 (s, 1H), 4.56 (s, 1H), 4.10 (d, J=3.7 Hz, 3H), 2.04-1.91 (m, 2H), 1.31 (s, 1H), 1.02 (t, J=7.3 Hz, 3H), 0.90 (s, 1H).

### 2.13: (S)—N-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-2-hydroxyethane-1-sulfonamide (Compound 130)

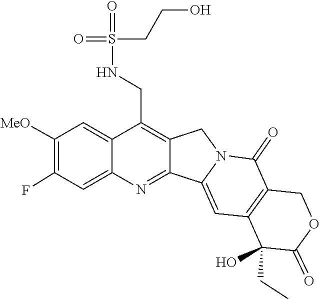 [SMILES: CC[C@@]1(O)C(=O)OCc2c1cc1n(c2=O)Cc2c-1nc1cc(F)c(OC)cc1c2CNS(=O)(=O)CCO]

The title compound was prepared according to General Procedure 3 starting from Compound 2.8 (8 mg) and 2-hydroxyethanesulfonyl chloride. Preparative HPLC purification was accomplished as described in General Procedure 9, eluting with a 15 to 50% CH₃CN/H₂O+0.1% TFA gradient to give the title compound as an off-white solid (2.2 mg, 22% yield).

LC/MS: Calc'd m/z=533.1 for C₂₄H₂₄FN₃O₈S found [M+H]⁺=534.2.

¹H NMR (300 MHz, DMSO-d6) δ 7.99 (d, J=12.2 Hz, 1H), 7.89-7.79 (m, 2H), 7.29 (s, 1H), 5.43 (s, 2H), 5.40 (s, 2H), 4.76 (d, J=6.4 Hz, 2H), 4.06 (s, 3H), 3.81 (t, J=6.3 Hz, 2H), 3.34 (t, J=6.3 Hz, 2H), 1.94-1.75 (m, 2H), 0.87 (d, J=7.4 Hz, 3H).

### 2.14: 4-nitrophenyl-(S)-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)carbamate (Compound 2.14)

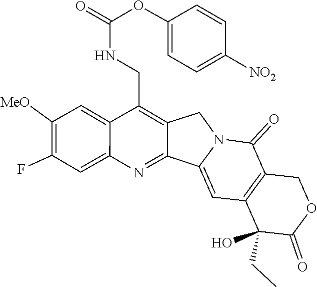 [SMILES: CC[C@@]1(O)C(=O)OCc2c1cc1n(c2=O)Cc2c-1nc1cc(F)c(OC)cc1c2CNC(=O)Oc1ccc([N+](=O)[O-])cc1]

The title PNP-carbamate intermediate compound was prepared according to the first step of General Procedure 4 starting from Compound 2.8 (65 mg) and using a 1:1 mixture of dimethylformamide and dichloromethane as the solvent. Flash purification was accomplished as described in General Procedure 9, using a 12 g C12 column and eluting with a 10 to 50% CH₃CN/H₂O+0.1% TFA gradient to give the title compound as an off-white solid (61 mg, 86% yield). This intermediate was divided and used to generate the following compounds.

LC/MS: Calc'd m/z=590.1 for C₂₉H₂₃FN₄O₉, found [M+H]⁺=591.2.

### 2.15: (S)-1-((4-ethyl-8-fluoro-4-hydroxy-9-methoxy-3,14-dioxo-3,4,12,14-tetrahydro-1H-pyrano[3′,4′: 6,7]indolizino[1,2-b]quinolin-11-yl)methyl)-3-methylurea (Compound 133)

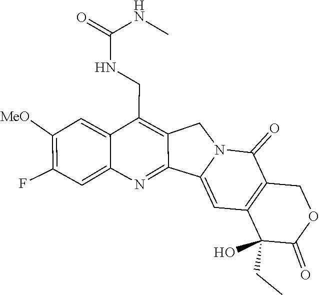 [SMILES: CC[C@@]1(O)C(=O)OCc2c1cc1n(c2=O)Cc2c-1nc1cc(F)c(OC)cc1c2CNC(=O)NC]

The title compound was prepared according to the second step of General Procedure 4 using Compound 2.14 (15 mg) as the PNP-carbamate and aqueous methyl amine (500 μL, 40 wt. % in water) as the primary amine. Preparative HPLC purification was accomplished as described in General Procedure 9, eluting with a 20 to 60% CH₃CN/H₂O+0.1% TFA gradient to give the title compound as an off-white solid (5.8 mg, 47% yield).

LC/MS: Calc'd m/z=482.2 for C₂₄H₂₃FN₄O₆, found [M+H]⁺=483.2.

¹H NMR (300 MHz, DMSO-d6) δ 8.00-7.87 (m, 2H), 7.31 (s, 1H), 5.48-5.39 (m, 3H), 4.81 (s, 3H), 2.56 (s, 3H), 1.93-1.81 (m, 2H), 0.89 (t, J=7.3 Hz, 3H).

### 2.16: (S)-1-(4-aminobenzyl)-3-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)urea (Compound 135)

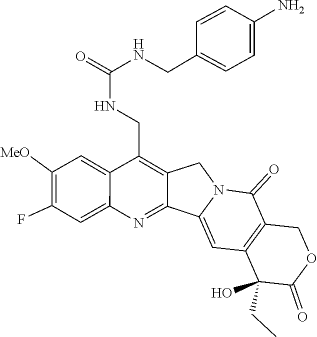 [SMILES: CC[C@@]1(O)C(=O)OCc2c1cc1n(c2=O)Cc2c-1nc1cc(F)c(OC)cc1c2CNC(=O)NCc1ccc(N)cc1]

The title compound was prepared according to the second step of General Procedure 4 using Compound 2.14 (15 mg) as the PNP-carbamate and 4-(aminomethyl)aniline as the primary amine. Preparative HPLC purification was accomplished as described in General Procedure 9, eluting with a 20 to 60% CH₃CN/H₂O+0.1% TFA gradient to give the title compound as an off-white solid (TFA salt, 2.1 mg, 12% yield).

LC/MS: Calc'd m/z=573.2 for C₃₀H₂₈FN₅O₆, found [M+H]⁺=574.2.

¹H NMR (300 MHz, MeOD) δ 7.79 (d, J=11.9 Hz, 1H), 7.74 (d, J=9.0 Hz, 1H), 7.59 (s, 1H), 7.43 (d, J=8.2 Hz, 2H), 7.25 (d, J=8.2 Hz, 2H), 5.61 (d, J=16.3 Hz, 1H), 5.52-5.35 (m, 3H), 4.98 (s, 2H), 4.39 (s, 2H), 4.01 (s, 3H), 2.03-1.93 (m, 2H), 1.03 (t, J=7.4 Hz, 3H).

### 2.17: (S)-1-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-3-(2-hydroxyethyl)urea (Compound 137)

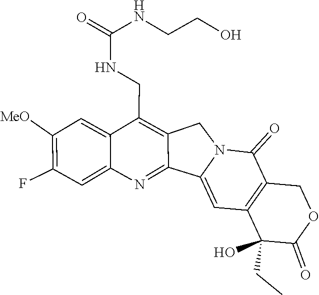 [SMILES: CC[C@@]1(O)C(=O)OCc2c1cc1n(c2=O)Cc2c-1nc1cc(F)c(OC)cc1c2CNC(=O)NCCO]

The title compound was prepared according to the second step of General Procedure 4 using Compound 2.14 (15 mg) as the PNP-carbamate and hydroxyethylamine as the primary amine. Preparative HPLC purification was accomplished as described in General Procedure 9, eluting with a 20 to 60% CH₃CN/H₂O+0.1% TFA gradient to give the title compound as an off-white solid (1.5 mg, 12% yield).

LC/MS: Calc'd m/z=512.2 for C₂₅H₂₅FN₄O₇, found [M+H]⁺=513.2.

¹H NMR (300 MHz, MeOD) δ 7.93 (d, J=12.1 Hz, 1H), 7.88 (d, J=9.2 Hz, 1H), 7.56 (s, 1H), 5.62 (d, J=16.2 Hz, 1H), 5.52 (s, 2H), 5.45 (d, J=16.3 Hz, 1H), 4.98 (s, 2H), 4.17 (s, 3H), 3.59 (t, J=5.6 Hz, 2H), 3.28 (t, J=5.6 Hz, 2H), 2.10-1.91 (m, 2H), 1.05 (t, J=7.3 Hz, 3H).

### Example 3: Preparation of Camptothecin Analogues Having Amino at the C10 Position

### 3.1: 5-bromo-4-fluoro-2-nitrobenzaldehyde (Compound 3.1)

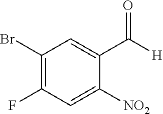 [SMILES: O=Cc1cc(Br)c(F)cc1[N+](=O)[O-]]

To a stirring solution of HNO₃ (121.2 mL, 67% purity, 2.0 eq.) in H₂SO₄ (500 mL) at 0° C. was added 3-bromo-4-fluorobenzaldehyde (180 g, 1.0 eq.). After the addition was complete, the ice bath was removed, and the reaction was allowed to stir for 5 h at 25° C. The mixture was poured into ice (5 L), filtered and then dried under vacuum. The title compound was obtained as a yellow solid (219 g).

¹H NMR (400 MHz, CDCl₃) δ 10.39 (s, 1H), 8.23 (d, J=6.8 Hz, 1H), 7.91 (d, J=7.6 Hz, 1H).

### 3.2: tert-butyl (2-fluoro-5-formyl-4-nitrophenyl)carbamate (Compound 3.2)

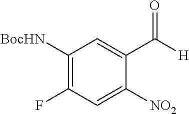 [SMILES: CC(C)(C)OC(=O)Nc1cc(C=O)c([N+](=O)[O-])cc1F]

A mixture of Compound 3.1 (219 g, 1.0 eq.), tert-butyl carbamate (124 g, 1.2 eq.), Cs₂CO₃ (575 g, 2 eq.), Pd₂(dba)₃ (40 g, 0.05 eq.) and XPhos (84 g, 0.2 eq.) in toluene (2000 mL) was degassed and purged with N₂ for three cycles. The mixture was then stirred at 90° C. for 15 h under N₂ atmosphere. The reaction mixture was diluted with H₂O (800 mL) and extracted with EtOAc (300 mL×2). The combined organic layers were washed with brine (200 mL×2), then dried over sodium sulfate, filtered and concentrated under reduced pressure. The residue was purified by column chromatography (SiO₂, petroleum ether: ethyl acetate=100:1 to 20:1) to afford the title compound as a yellow solid (140 g, 56% yield).

¹H NMR (400 MHz, DMSO-d6) δ 10.24 (s, 1H), 9.94 (s, 1H), 8.42 (d, J=7.6 Hz, 1H), 8.16 (d, J=10.8 Hz, 1H), 1.50 (s, 9H)

### 3.3: tert-butyl (4-amino-2-fluoro-5-formylphenyl)carbamate (Compound 3.3)

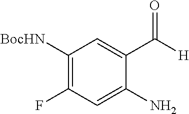 [SMILES: CC(C)(C)OC(=O)Nc1cc(C=O)c(N)cc1F]

To a solution of Compound 3.2 (100 g, 1.0 eq.) in H₂O (300 mL) and EtOH (1200 mL) was added NH₄C₁ (30.5 g, 1.62 eq.). Iron (78.6 g, 4.0 eq.) was added in portions at 80° C. The mixture was stirred at 80° C. for 6 h. The mixture was filtered, water was added to the filtrate, and the resulting mixture was extracted with ethyl acetate. The organic layer was washed with brine, dried over sodium sulfate, and concentrated under vacuum. The residue was purified by column chromatography (SiO₂, Petroleum ether: ethyl acetate=1:0 to 0: 1), TLC (petroleum ether) to afford the title compound as a yellow solid (19.0 g, 21% yield).

LC/MS: Calc'd m/z=254.1 for C₁₂H₁₅FN₂₀₃, found [M+H]⁺=255.0.

¹H NMR (400 MHz, DMSO-d6) δ 9.73 (s, 1H), 8.57 (s, 1H), 7.58 (d, J=4.8 Hz, 1H), 7.21 (s, 2H), 6.53 (d, J=12.8 Hz, 1H), 1.43 (s, 9H).

### 3.4: tert-butyl (S)-(4-ethyl-8-fluoro-4-hydroxy-3,14-dioxo-3,4,12,14-tetrahydro-1H-pyrano[3′,4′:6,7]indolizino[1,2-b]quinolin-9-yl)carbamate (Compound 3.4)

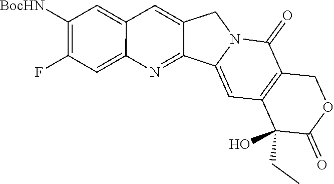 [SMILES: CC[C@@]1(O)C(=O)OCc2c1cc1n(c2=O)Cc2cc3cc(NC(=O)OC(C)(C)C)c(F)cc3nc2-1]

A mixture of Compound 3.3 (4.20 g, 1.2 eq.), (S)-4-ethyl-4-hydroxy-7,8-dihydro-1H-pyrano[3,4-f]indolizine-3,6,10(4H)-trione (3.5 g, 1 eq.) and TsOH (monohydrate, 253 mg, 0.1 eq.) in toluene (350 mL) was stirred at 110° C. for 2 hrs. The reaction solution was cooled to 25° C., filtered, the solid was washed with methyl-t-butyl ether (30 mL) and then dried under vacuum. The title compound was obtained as a yellow solid (4.5 g, 62% yield).

LC/MS: Calc'd m/z=481.2 for C₂₅H₂₄FN₃O₆, found [M+H]⁺=482.1.

¹H NMR (400 MHz, DMSO-d6) δ 9.49 (s, 1H), 8.65 (s, 1H), 8.43 (d, J=8.4 Hz, 1H), 7.95 (d, J=12.0 Hz, 1H), 7.30 (s, 1H), 6.51 (s, 1H), 5.42 (s, 2H), 5.25 (s, 2H), 1.80-1.92 (m, 2H), 1.52 (s, 9H), 0.88 (t, J=7.2 Hz, 3H)

### 3.5: tert-butyl (S)-(4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)carbamate (Compound 3.5)

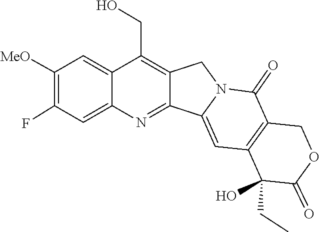 [SMILES: CC[C@@]1(O)C(=O)OCc2c1cc1n(c2=O)Cc2c-1nc1cc(F)c(OC)cc1c2CO]

To a mixture of Compound 3.4 (4.00 g) in MeOH (360 mL) was added a solution of FeSO₄ (heptahydrate, 1.2 g), H₂SO₄ (280 μL) in H₂O (4 mL). The reaction mixture was heated at 65° C. while H₂O₂ (24 mL, 30% purity) was added dropwise over 30 min and then stirred 0.5 h. The reaction solution was cooled to 25° C., then filtered to provide the title compound as a yellow solid (1.53 g, 33.2% yield). To the filtrate was added H₂O (400 mL), then quenched with saturated aqueous Na₂S₂O₃. The pH was adjusted to 7-8 with saturated aqueous Na₂CO₃ then the solution was concentrated and filtered. The solid was triturated with MeOH (30 mL) at 55° C. for 1 h, then filtered, to provide a second batch of the title compound as a brown solid (1.09 g, 26% yield).

LC/MS: Calc'd m/z=511.2 for C₂₆H₂₆FN₃O₇, found [M+H]⁺=512.2.

¹H NMR (300 MHz, d6-DMSO) δ 9.47 (s, 1H), 8.47 (d, J=7.6 Hz, 1H), 7.94 (d, J=12.0 Hz, 1H), 7.29 (d, J=1.6 Hz, 1H), 6.49 (s, 1H), 5.86-5.76 (m, 1H), 5.42 (s, 2H), 5.38 (s, 2H), 5.16 (d, J=4.4 Hz, 2H), 1.90-1.83 (m, 2H), 1.52 (s, 9H), 0.88 (t, J=6.4 Hz, 3H).

### 3.6: tert-butyl(S)-(4-ethyl-8-fluoro-11-formyl-4-hydroxy-3,14-dioxo-3,4,12,14-tetrahydro-1H-pyrano[3′,4′:6,7]indolizino[1,2-b]quinolin-9-yl)carbamate (Compound 3.6)

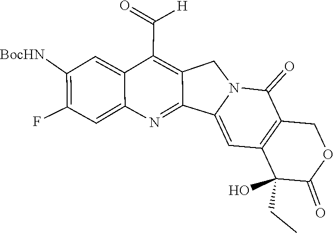 [SMILES: CC[C@@]1(O)C(=O)OCc2c1cc1n(c2=O)Cc2c-1nc1cc(F)c(NC(=O)OC(C)(C)C)cc1c2C=O]

In a 50 mL round-bottom flask containing Compound 3.5 (150 mg, 0.293 mmol) was added DCM (2.9 mL) followed by Dess-Martin periodinane (0.56 g, 1.32 mmol) and water (15.8 μL, 0.88 mmol). This solution was stirred at room temperature for 18 h then diluted with DCM, washed with saturated aqueous NaHCO₃ and brine. The layers were separated, and the combined organic layers were evaporated onto celite. Flash purification was accomplished as described in General Procedure 9, using a 10 g silica column and eluting with 0 to 10% DCM/MeOH to give the title product as an orange powder (42.5 mg, 28%).

LC/MS: Calc'd m/z=509.2 for C₂₆H₂₄FN₃O₇, found [M+H]⁺=510.4.

¹H NMR (300 MHz, Acetone-d6) δ 11.10 (s, 1H), 9.68 (d, J=8.6 Hz, 1H), 8.81 (s, 1H), 8.04 (d, J=11.9 Hz, 1H), 7.63 (s, 1H), 5.73 (s, 2H), 5.69 (d, J=16.2 Hz, 1H), 5.42 (d, J=16.2 Hz, 1H), 2.02-1.95 (m, 2H), 8.47 (d, J=7.6 Hz, 1H), 7.94 (d, J=12.0 Hz, 1H), 7.29 (d, J=1.6 Hz, 1H), 6.49 (s, 1H), 5.86-5.76 (m, 1H), 5.42 (s, 2H), 5.38 (s, 2H), 5.16 (d, J=4.4 Hz, 2H), 1.90-1.83 (m, 2H), 1.52 (s, 9H), 0.88 (t, J=6.4 Hz, 3H).

### 3.7: (S)-9-amino-4-ethyl-8-fluoro-4-hydroxy-1,12-dihydro-14H-pyrano[3′, 4′:6,7]indolizino [1,2-b]quinoline-3,14(4H)-dione (Compound 140)

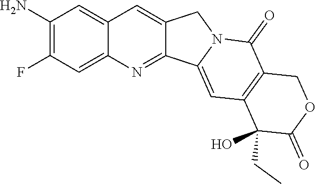 [SMILES: CC[C@@]1(O)C(=O)OCc2c1cc1n(c2=O)Cc2cc3cc(N)c(F)cc3nc2-1]

The title compound was prepared according to General Procedure 6 starting from Compound 3.4 (40 mg) to give the title compound as a red solid (TFA salt, 36 mg, 87% yield).

LC/MS: Calc'd m/z=381.1 for C₂₀H₁₆FN₃O₄, found [M+H]⁺=382.2.

¹H NMR (300 MHz, DMSO) δ 8.28 (s, 1H), 7.72 (d, J=12.5 Hz, 1H), 7.21 (d, J=7.3 Hz, 1H), 5.43 (d, J=16.2 Hz, 1H), 5.34 (d, J=16.2 Hz, 1H), 5.17 (s, 2H), 1.92-1.74 (m, 2H), 0.88 (t, J=7.3 Hz, 3H).

### 3.8: (S)-9-amino-4-ethyl-8-fluoro-4-hydroxy-11-(hydroxymethyl)-1,12-dihydro-14H-pyrano[3′,4′: 6,7]indolizino[1,2-b]quinoline-3,14(4H)-dione (Compound 141)

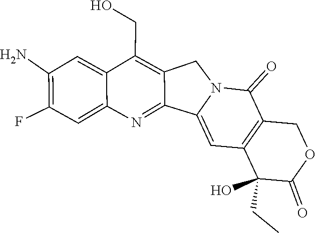 [SMILES: CC[C@@]1(O)C(=O)OCc2c1cc1n(c2=O)Cc2c-1nc1cc(F)c(N)cc1c2CO]

The title compound was prepared according to General Procedure 6 starting from Compound 3.5 (5 mg) to give the title compound as a red solid (TFA salt, 4.1 mg, 78% yield).

LC/MS: Calc'd m/z=411.2 for C₂₁H₁₈FN₃O₅, found [M+H]⁺=412.2.

¹H NMR (300 MHz, MeOD) δ 7.71 (d, J=12.2 Hz, 1H), 7.60 (s, 1H), 7.29 (d, J=9.5 Hz, 1H), 5.61 (d, J=16.3 Hz, 1H), 5.47 (s, 2H), 5.40 (d, J=16.3 Hz, 1H), 5.25 (s, 2H), 2.03-1.94 (m, 2H), 1.03 (t, J=7.4 Hz, 3H).

### 3.9: tert-butyl (S)-(11-(chloromethyl)-4-ethyl-8-fluoro-4-hydroxy-3,14-dioxo-3,4,12,14-tetrahydro-1H-pyrano[3′,4′:6,7]indolizino[1,2-b]quinolin-9-yl)carbamate (Compound 3.9)

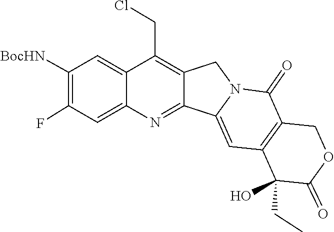 [SMILES: CC[C@@]1(O)C(=O)OCc2c1cc1n(c2=O)Cc2c-1nc1cc(F)c(NC(=O)OC(C)(C)C)cc1c2CCl]

To a stirring solution of Compound 3.5 (100 mg) in dichloromethane (5 mL) was added a solution of thionyl chloride (14 μL) in dichloromethane (0.1 mL). After 1 h, additional thionyl chloride (14 μL) in dichloromethane (0.1 mL) was added. After another 1 h the reaction was diluted with dichloromethane (10 mL) and toluene (1 mL) then concentrated in vacuo to provide the title compound as a red solid that was used in subsequent reactions without additional purification.

LC/MS: Calc'd m/z=529.1 for C₂₆H₂₅C₁FN₃O₆, found [M+H]⁺=530.2.

### 3.10: tert-butyl (S)-(11-(aminomethyl)-4-ethyl-8-fluoro-4-hydroxy-3,14-dioxo-3,4,12,14-tetrahydro-1H-pyrano[3′,4′:6,7]indolizino[1,2-b]quinolin-9-yl)carbamate (Compound 3.10)

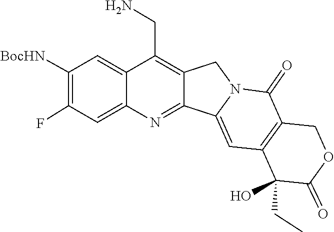 [SMILES: CC[C@@]1(O)C(=O)OCc2c1cc1n(c2=O)Cc2c-1nc1cc(F)c(NC(=O)OC(C)(C)C)cc1c2CN]

To Compound 3.9 (100 mg) in ethanol (500 μL) was added hexamethylenetetramine (79 mg) then DIPEA (99 μL). This solution was heated at 60° C. for 16 h then concentrated to dryness in vacuo. Flash purification was accomplished as described in General Procedure 9, using a 12 g C18 column and eluting with a 10 to 50% CH₃CN/H₂O+0.1% TFA gradient to give the title compound as an off-white solid (TFA salt, 29 mg, 24% yield).

LC/MS: Calc'd m/z=510.2 for C₂₆H₂₇FN₄O₆, found [M+H]⁺=511.4.

¹H NMR (300 MHz, MeOD) δ 8.88 (d, J=8.2 Hz, 1H), 7.96 (d, J=11.9 Hz, 1H), 7.62 (s, 1H), 5.60 (d, J=16.4 Hz, 1H), 5.48 (s, 2H), 5.41 (d, J=16.4 Hz, 1H), 4.80 (s, 2H), 2.07-1.89 (m, 2H), 1.64 (s, 9H), 1.02 (t, J=7.3 Hz, 3H).

### 3.11: (S)-9-amino-11-(aminomethyl)-4-ethyl-8-fluoro-4-hydroxy-1,12-dihydro-14H-pyrano[3′,4′:6,7]indolizino[1,2-b]quinoline-3,14(4H)-dione (Compound 145)

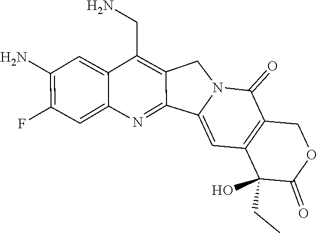 [SMILES: CC[C@@]1(O)C(=O)OCc2c1cc1n(c2=O)Cc2c-1nc1cc(F)c(N)cc1c2CN]

The title compound was prepared according to General Procedure 6 starting from Compound 3.10 (2.1 mg) to give the title compound as a red solid (TFA salt, 1.8 mg, 100% yield).

LC/MS: Calc'd m/z=410.1 for C₂₁H₁₉FN₄O₄, found [M+H]⁺=411.2.

¹H NMR (300 MHz, MeOD) δ 7.82 (d, J=12.1 Hz, 1H), 7.60 (s, 1H), 7.37 (d, J=9.1 Hz, 1H), 5.61 (d, J=16.3 Hz, 1H), 5.42 (s, 2H), 5.41 (d, J=16.3 Hz, 1H), 4.69 (s, 2H), 2.08-1.94 (m, 2H), 1.03 (t, J=7.4 Hz, 3H).

### Example 3.12: (S)-9-amino-4-ethyl-8-fluoro-4-hydroxy-11-(morpholinomethyl)-1,12-dihydro-14H-pyrano[3′,4′:6,7]indolizino[1,2-b]quinoline-3,14(4H)-dione (Compound 3.12)

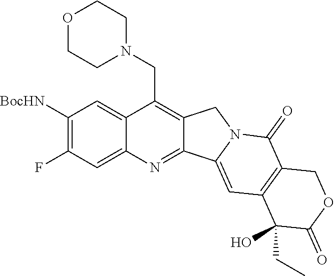 [SMILES: CC[C@@]1(O)C(=O)OCc2c1cc1n(c2=O)Cc2c-1nc1cc(F)c(NC(=O)OC(C)(C)C)cc1c2CN1CCOCC1]

The title compound was prepared according to General Procedure 1 starting from Compound 3.9 (150 mg) and morpholine. Preparative HPLC purification was accomplished as described in General Procedure 9, eluting with a 10 to 60% CH₃CN/H₂O+0.1% TFA gradient to give the title compound as a red solid (TFA salt, 103 mg, 52% yield).

LC/MS: Calc'd m/z=580.2 for C₃₀H₃₃FN₄O₇, found [M+H]⁺=581.4.

¹H NMR (300 MHz, MeOD) δ 9.06 (d, J=8.3 Hz, 1H), 7.93 (d, J=12.0 Hz, 1H), 7.66 (s, 1H), 5.63 (d, J=16.3 Hz, 1H), 5.51 (s, 2H), 5.43 (d, J=16.4 Hz, 1H), 4.92 (s, 2H), 3.84 (s, 4H), 3.10 (s, 4H), 1.99 (d, J=5.5 Hz, 2H), 1.63 (s, 9H), 1.03 (t, J=7.4 Hz, 3H).

### 3.13: (S)-9-amino-4-ethyl-8-fluoro-4-hydroxy-11-(morpholinomethyl)-1,12-dihydro-14H-pyrano[3′,4′:6,7]indolizino[1,2-b]quinoline-3,14(4H)-dione (Compound 142)

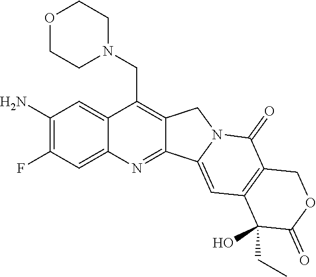 [SMILES: CC[C@@]1(O)C(=O)OCc2c1cc1n(c2=O)Cc2c-1nc1cc(F)c(N)cc1c2CN1CCOCC1]

The title compound was prepared according to General Procedure 6 starting from Compound 3.12 (45 mg) to give the title compound as a red solid (TFA salt, 37 mg, 99% yield).

LC/MS: Calc'd m/z=480.2 for C₂₅H₂₅FN₄O₅, found [M+H]⁺=481.4.

¹H NMR (300 MHz, MeOD) δ 7.73 (d, J=12.0 Hz, 1H), 7.54 (s, 1H), 7.48 (d, J=9.2 Hz, 1H), 5.60 (d, J=16.3 Hz, 1H), 5.47-5.34 (m, 3H), 4.65 (s, 2H), 3.91-3.85 (m, 4H), 3.30-3.24 (m, 4H), 2.08-1.91 (m, 2H), 1.02 (t, J=7.3 Hz, 3H).

### 3.14: (S)-9-amino-4-ethyl-8-fluoro-4-hydroxy-11-(piperidin-1-ylmethyl)-1,12-dihydro-14H-Pyrano[3′,4′ 6,7]indolizino[1,2-b]quinoline-3,14(4H)-dione (Compound 148)

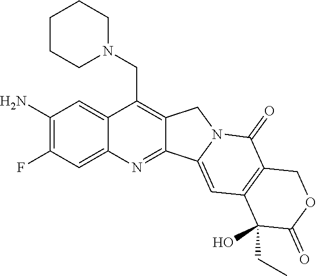 [SMILES: CC[C@@]1(O)C(=O)OCc2c1cc1n(c2=O)Cc2c-1nc1cc(F)c(N)cc1c2CN1CCCCC1]

To a 5 mL flask containing Compound 3.6 (37 mg, 0.067 mmol) was added dichloromethane (1.45 mL) followed by acetic acid (18.69 μL, 0.327 mmol), piperidine (21.52 μL, 0.218 mmol), and sodium triacetoxyborohydride (23.0 mg, 0.109 mmol). This solution was then stirred at room temperature for 2 h, quenched by the addition of water+0.1% TFA and DMF (1:1, 1.0 mL), and partially evaporated. Purification was accomplished as described in General Procedure 9, using a 12 g C18 flash column and eluting with a 5 to 40% CH₃CN/H₂O+0.1% TFA gradient to give the Boc-protected intermediate as a yellow powder. This intermediate was then deprotected according to General Procedure 6 to give the title compound as a yellow solid (TFA salt, 32.5 mg, 98% yield).

LC/MS: Calc'd m/z=478.2 for C₂₆H₂₇FN₄O₄, found [M+H]⁺=479.4.

¹H NMR (300 MHz, MeOD) δ 7.78 (d, J=12.1 Hz, 1H), 7.56 (s, 1H), 7.41 (d, J=9.1 Hz, 1H), 5.60 (d, J=16.4 Hz, 1H), 5.47-5.35 (m, 3H), 4.86 (s, 2H), 3.80-3.68 (m, 2H), 3.28-3.19 (m, 2H), 2.02-1.68 (m, 8H), 1.01 (t, J=7.4 Hz, 3H).

### 3.15: (S)-9-amino-4-ethyl-8-fluoro-4-hydroxy-11-((4-methylpiperazin-1-yl)methyl)-1,12-dihydro-14H-pyrano[3′,4′: 6,7]indolizino[1,2-b]quinoline-3,14(4H)-dione (Compound 149)

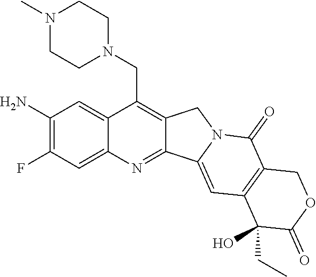 [SMILES: CC[C@@]1(O)C(=O)OCc2c1cc1n(c2=O)Cc2c-1nc1cc(F)c(N)cc1c2CN1CCN(C)CC1]

To a 2 mL vial containing Compound 3.6 (15 mg, 0.029 mmol) was added dichloromethane (0.59 mL), acetic acid (7.58 μL, 0.132 mmol), and N-methylpiperazine (4.90 μL, 0.044 mmol). This solution was stirred at room temperature for 4 h then sodium triacetoxyborohydride (7.8 mg, 0.037 mmol) was then added and stirred for an additional 45 min. Excess hydride was then quenched by the addition of a 0.1% aqueous TFA solution (0.5 mL). Purification was accomplished as described in General Procedure 9 using a 12 g C18 flash column and eluting with a 5 to 40% CH₃CN/H₂O+0.1% TFA gradient to give the Boc-protected intermediate as a yellow powder. This intermediate was deprotected according to General Procedure 6 to give the title product as a yellow solid (TFA salt, 1.5 mg, 7.1% yield).

LC/MS: Calc'd m/z=493.2 for C₂₆H₂₈FN₅₀₄, found [M+H]⁺=494.4.

¹H NMR (300 MHz, MeOD) δ 7.68 (d, J=12.2 Hz, 1H), 7.56 (s, 1H), 7.53 (d, J=9.5 Hz, 1H), 5.60 (d, J=16.3 Hz, 1H), 5.45-5.30 (m, 3H), 4.15 (s, 2H), 3.55-3.44 (m, 2H), 3.18-3.07 (m, 2H), 2.93 (s, 3H), 2.70-2.51 (m, 2H), 2.03-1.89 (m, 2H), 1.02 (t, J=7.4 Hz, 3H).

### 3.16: (S)-9-amino-4-ethyl-8-fluoro-4-hydroxy-11-((4-(phenylsulfonyl)piperazin-1-yl)methyl)-1,12-dihydro-14H-pyrano[3′,4′:6,7]indolizino[1,2-b]quinoline-3,14(4H)-dione (Compound 153)

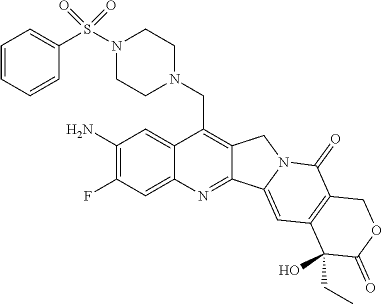 [SMILES: CC[C@@]1(O)C(=O)OCc2c1cc1n(c2=O)Cc2c-1nc1cc(F)c(N)cc1c2CN1CCN(S(=O)(=O)c2ccccc2)CC1]

The Boc-protected precursor of the title compound was prepared according to General Procedure 1 starting from Compound 3.9 (10 mg) and 1-(phenylsulfonyl)piperazine. Preparative HPLC was accomplished as described in General Procedure 9, eluting with a 35 to 44% CH₃CN/H₂O+0.1% TFA gradient to give the Boc-protected intermediate as a yellow powder. This intermediate was then deprotected according to General Procedure 6 to give the title compound (TFA salt, 2.4 mg, 17% yield over 2 steps).

LC/MS: Calc'd m/z=619.2 for C₃₁H₃₀FN₅O₆S, found [M+H]⁺=520.4.

¹H NMR (300 MHz, MeOD) δ 7.81-7.60 (m, 7H), 7.34 (s, 1H), 5.51 (d, J=16.4 Hz, 1H), 5.35 (d, J=16.4 Hz, 1H), 5.22 (s, 2H), 4.10 (s, 2H), 3.15-3.02 (m, 4H), 2.79-2.71 (m, 4H), 2.00-1.93 (m, 2H), 1.00 (t, J=7.4 Hz, 3H).

### 3.17: (S)—N-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)acetamide (Compound 147)

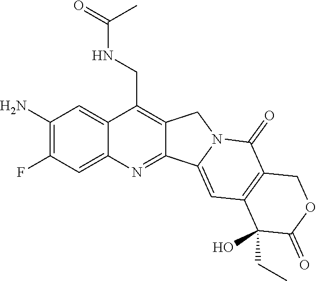 [SMILES: CC[C@@]1(O)C(=O)OCc2c1cc1n(c2=O)Cc2c-1nc1cc(F)c(N)cc1c2CNC(C)=O]

The title compound was prepared according to General Procedure 2 followed by General Procedure 6 starting from Compound 3.10 (8 mg) and acetic acid. Preparative HPLC purification of the intermediate Boc-protected compound was accomplished as described in General Procedure 9, eluting with a 10 to 60% CH₃CN/H₂O+0.1% TFA gradient. The title compound was obtained as a red solid (4.0 mg, 56% yield).

LC/MS: Calc'd m/z=452.2 for C₂₃H₂₁FN₄O₅, found [M+H]⁺=453.2.

¹H NMR (300 MHz, MeOD) δ 7.69 (d, J=12.1 Hz, 1H), 7.56 (s, 1H), 7.38 (d, J=9.3 Hz, 1H), 5.59 (d, J=16.3 Hz, 1H), 5.44-5.33 (m, 3H), 4.85 (s, 3H), 2.03 (s, 3H), 2.00-1.84 (m, 2H), 1.03 (t, J=7.4 Hz, 3H).

### 3.18: (S)—N-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)methanesulfonamide (Compound 146)

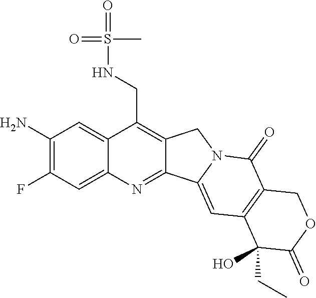 [SMILES: CC[C@@]1(O)C(=O)OCc2c1cc1n(c2=O)Cc2c-1nc1cc(F)c(N)cc1c2CNS(C)(=O)=O]

The title compound was prepared according to General Procedure 3 followed by General Procedure 6 starting from Compound 3.10 (8 mg) and methane sulfonyl chloride. Preparative HPLC purification of the intermediate Boc-protected compound was accomplished as described in General Procedure 9, eluting with a 10 to 60% CH₃CN/H₂O+0.1% TFA gradient. The title compound was obtained as a red solid (4.4 mg, 57% yield).

LC/MS: Calc'd m/z=488.1 for C₂₂H₂₁FN₄O₆S, found [M+H]⁺=489.2.

¹H NMR (300 MHz, MeOD) δ 7.74 (d, J=12.2 Hz, 1H), 7.60 (s, 1H), 7.49 (d, J=9.3 Hz, 1H), 5.61 (d, J=16.2 Hz, 1H), 5.45 (s, 2H), 5.40 (d, J=16.2 Hz, 1H), 4.78 (s, 2H), 3.05 (s, 3H), 2.08-1.94 (m, 2H), 1.03 (t, J=7.4 Hz, 3H).

### 3.19: (S)—N-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-2-hydroxyethane-1-sulfonamide (Compound 150)

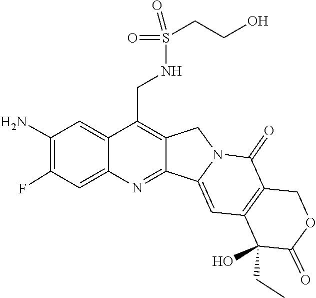 [SMILES: CC[C@@]1(O)C(=O)OCc2c1cc1n(c2=O)Cc2c-1nc1cc(F)c(N)cc1c2CNS(=O)(=O)CCO]

The title compound was prepared according to General Procedure 3 followed by General Procedure 6 starting from Compound 3.10 (6 mg) and 2-hydroxyethanesulfonyl chloride. Preparative HPLC purification of the intermediate Boc-protected compound was accomplished as described in General Procedure 9, eluting with a 10 to 60% CH₃CN/H₂O+0.1% TFA gradient. The title compound was obtained as a red solid (1 mg, 16% yield).

LC/MS: Calc'd m/z=518.5 for C₂₃H₂₃FN₄O₇S, found [M+H]⁺=519.5.

¹H NMR (300 MHz, 10% D₂O/CD₃CN) δ 7.77-7.61 (m, 1H), 7.48-7.30 (m, 2H), 5.53 (d, J=16.3 Hz, 1H), 5.31 (d, J=15.4 Hz, 3H), 4.69 (s, 2H), 3.97 (dd, J=6.6, 4.9 Hz, 2H), 3.39 (t, J=5.8 Hz, 2H), 2.93 (s, 1H), 1.99-1.83 (m, 2H), 0.94 (t, J=7.3 Hz, 3H).

### 3.20: 4-nitrophenyl (S)-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)carbamate (Compound 3.20)

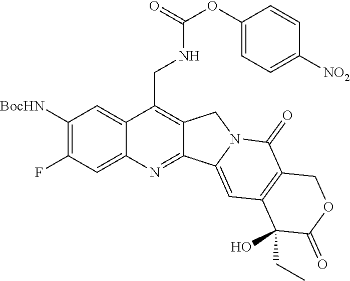 [SMILES: CC[C@@]1(O)C(=O)OCc2c1cc1n(c2=O)Cc2c-1nc1cc(F)c(NC(=O)OC(C)(C)C)cc1c2CNC(=O)Oc1ccc([N+](=O)[O-])cc1]

To a solution of Compound 3.10 (10 mg, 0.02 mmol) in DMF (400 μL, 0.05 M) was added 4-nitrophenyl carbonate (12 mg, 0.04 mmol) and diisopropylethylamine (6.8 μL, 0.04 mmol). This solution was stirred at room temperature for ˜30 min, then used directly in subsequent reactions.

### 3.21: Methyl (S)-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)carbamate (Compound 143)

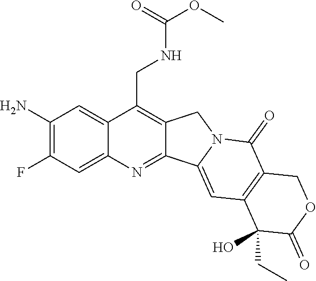 [SMILES: CC[C@@]1(O)C(=O)OCc2c1cc1n(c2=O)Cc2c-1nc1cc(F)c(N)cc1c2CNC(=O)OC]

The title compound was prepared by addition of MeOH (100 μL) to 200 ul of the solution of Compound 3.20. This solution was stirred at room temperature for 30 min. Preparative HPLC purification of the intermediate Boc-protected compound was accomplished as described in General Procedure 9, eluting with a 10 to 60% CH₃CN/H₂O+0.1% TFA gradient. The title compound was obtained according to General Procedure 6 as a red solid (2.1 mg, 47% yield).

LC/MS: Calc'd m/z=468.4 for C₂₃H₂₁FN₄O₆, found [M+H]⁺=468.3.

¹H NMR (300 MHz, 10% D₂O/CD₃CN) δ 7.72 (d, J=12.2 Hz, 1H), 7.41 (d, J=18.1 Hz, 1H), 6.96 (s, 1H), 5.52 (d, J=3.6 Hz, 1H), 5.39-5.23 (m, 3H), 4.82 (s, 1H), 4.73 (s, 1H), 3.63 (d, J=1.2 Hz, 3H), 1.56 (s, 3H), 1.27 (s, 2H), 0.94 (t, J=7.4 Hz, 3H).

### 3.22: (S)-1-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-3-methylurea (Compound 144)

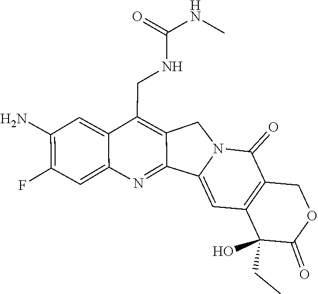 [SMILES: CC[C@@]1(O)C(=O)OCc2c1cc1n(c2=O)Cc2c-1nc1cc(F)c(N)cc1c2CNC(=O)NC]

The title compound was prepared by addition of methylamine hydrochloride (10 mg) to 200 μL of the solution of Compound 3.20, followed by iPr₂NEt (5 μL). This solution was stirred at room temperature for 30 min. Preparative HPLC purification of the intermediate Boc-protected compound was accomplished as described in General Procedure 9, eluting with a 10 to 60% CH₃CN/H₂O+0.1% TFA gradient. The title compound was obtained according to General Procedure 6 as a red solid (2.9 mg, 64.5% yield).

LC/MS: Calc'd m/z=467.5 for C₂₃H₂₁FN₅O₅, found [M+H]⁺=468.5.

¹H NMR (300 MHz, 10% D₂O/CD₃CN) δ 8.13 (d, J=9.2 Hz, 1H), 7.92 (s, 1H), 7.73 (d, J=12.3 Hz, 1H), 7.52-7.35 (m, 2H), 6.94 (d, J=9.2 Hz, 2H), 5.55 (d, J=16.5 Hz, 2H), 5.44-5.27 (m, 4H), 4.85 (s, 2H), 4.78 (s, 1H), 1.56 (d, J=2.5 Hz, 3H), 1.27 (s, 2H), 0.93 (q, J=11.7, 9.5 Hz, 3H).

### 3.23: (S)-1-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-3-(2-hydroxyethyl)urea (Compound 151)

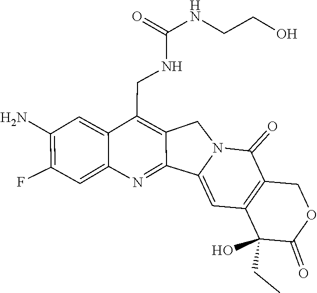 [SMILES: CC[C@@]1(O)C(=O)OCc2c1cc1n(c2=O)Cc2c-1nc1cc(F)c(N)cc1c2CNC(=O)NCCO]

The title compound was prepared by addition of ethanolamine (100 μL) to 200 μL of the solution of Compound 3.20. This solution was stirred at room temperature for 30 min. Preparative HPLC purification of the intermediate Boc-protected compound was accomplished as described in General Procedure 9, eluting with a 10 to 60% CH₃CN/H₂O+0.1% TFA gradient. The title compound was obtained according to General Procedure 6 as a red solid (0.5 mg, 8.5% yield).

LC/MS: Calc'd m/z=497.5 for C₂₄H₂₄FN₅O₆, found [M+H]⁺=498.5.

¹H NMR (300 MHz, 10% D₂O/CD₃CN) δ 7.77-7.61 (m, 1H), 7.48-7.30 (m, 2H), 5.53 (d, J=16.3 Hz, 1H), 5.31 (d, J=15.4 Hz, 1H), 5.19 (s, 2H), 4.69 (s, 2H), 3.97 (dd, J=6.6, 4.9 Hz, 2H), 3.39 (t, J=5.8 Hz, 2H), 2.93 (s, 1H), 2.01-1.83 (m, 2H), 0.94 (t, J=7.3 Hz, 3H).

### 3.24: (S)-9-amino-11-(azidomethyl)-4-ethyl-8-fluoro-4-hydroxy-1,12-dihydro-14H-pyrano[3′,4′ 6,7]indolizino[1,2-b]quinoline-3,14(4H)-dione (Compound 152)

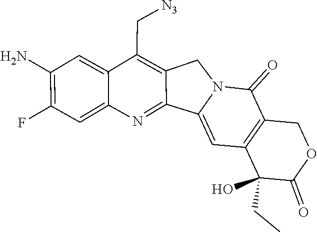 [SMILES: CC[C@@]1(O)C(=O)OCc2c1cc1n(c2=O)Cc2c-1nc1cc(F)c(N)cc1c2CN=[N+]=[N-]]

To a stirring solution of Compound 3.5 (100 mg) in 2 mL dichloromethane was added thionyl chloride (35 μL, 2.5 eq.). The solution was stirred at room temperature for 20 min, then additional thionyl chloride (35 μL, 2.5 eq.) was added. After 20 minutes, toluene (1 mL) was added, and the reaction mixture was concentrated in vacuo. The crude solid was suspended in DMSO (1 mL) and sodium azide (19 mg, 1.5 eq.) was added. This solution was stirred at room temperature for 16 h. Purification was accomplished as described in General Procedure 9, eluting with a 5 to 50% CH₃CN/H₂O+0.1% TFA gradient to give the title compound as an off-white solid (20 mg, 23% yield).

LC/MS: Calc'd m/z=436.1 for C₂₁H₁₇FN₆₀₄, found [M+H]⁺=437.2.

¹H NMR (300 MHz, MeOD) δ 7.75 (d, J=12.2 Hz, 1H), 7.60 (s, 1H), 7.38 (d, J=9.3 Hz, 1H), 5.61 (d, J=16.3 Hz, 1H), 5.46-5.35 (m, 3H), 5.07 (s, 2H), 2.03-1.97 (m, 2H), 1.03 (t, J=7.3 Hz, 3H).

### 3.25: (S)—N-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)acetamide (Compound 164)

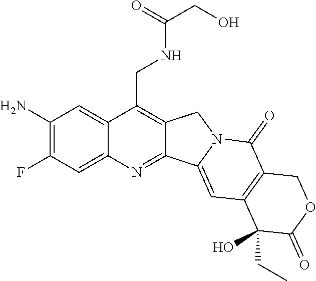 [SMILES: CC[C@@]1(O)C(=O)OCc2c1cc1n(c2=O)Cc2c-1nc1cc(F)c(N)cc1c2CNC(=O)CO]

The title compound was prepared according to General Procedure 2 starting from Compound 145 (10 mg) and glycolic acid. Preparative HPLC purification was accomplished as described in General Procedure 9, eluting with a 10 to 45% CH₃CN/H₂O+0.1% TFA gradient. The title compound was obtained as a yellow solid (6.9 mg, 60% yield).

LC/MS: Calc'd m/z=468.1 for C₂₃H₂₁FN₄O₆, found [M+H]⁺=469.2.

¹H NMR (300 MHz, MeOD) 7.70 (d, J=12.2 Hz, 1H), 7.60 (s, 1H), 7.42 (d, J=9.4 Hz, 1H), 5.62 (d, J=16.3 Hz, 1H), 5.43 (s, 2H), 5.36 (d, J=16.2 Hz, 1H), 4.95 (d, J=5.9 Hz, 2H), 4.08 (s, 2H), 2.04-1.90 (m, 1H), 1.03 (t, J=7.4 Hz, 3H).

### 3.26: (S)-1-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-3-methylthiourea (Compound 161)

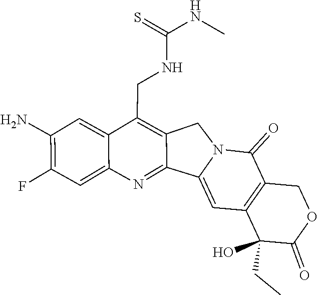 [SMILES: CC[C@@]1(O)C(=O)OCc2c1cc1n(c2=O)Cc2c-1nc1cc(F)c(N)cc1c2CNC(=S)NC]

To a solution of Compound 145 (9 mg, 1.0 eq.) in DMF (1 mL) was added thiocarbonyldiimidazole (6 mg, 1.5 eq.) then DIPEA (8 μL, 2.0 eq.). The resulting solution was stirred at 25° C. for 2 h, after which complete conversion to the isothiocyanate intermediate was observed. Methylammonium chloride (3 mg, 2.0 eq.) was then added and the reaction mixture was heated at 60° C. for 30 min. Preparative HPLC purification was accomplished as described in General Procedure 9, eluting with a 10 to 45% CH₃CN/H₂O+0.1% TFA gradient. The title compound was obtained as a yellow solid (2.3 mg, 22% yield).

LC/MS: Calc'd m/z=483.1 for C₂₃H₂₂FN₅₀₄S found [M+H]⁺=484.2.

¹H NMR (300 MHz, MeOD) δ 7.70 (d, J=12.0 Hz, 1H), 7.60 (s, 1H), 7.38 (d, J=9.3 Hz, 1H), 5.62 (d, J=16.2 Hz, 1H), 5.36 (s, 2H), 5.31 (d, J=16.2 Hz, 1H), 5.30 (s, 2H), 3.04 (s, 3H), 1.99-1.90 (m, 2H), 1.02 (t, J=7.4 Hz, 3H).

### 3.27: S-(2-hydroxyethyl)-(S)-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)carbamothioate (Compound 160)

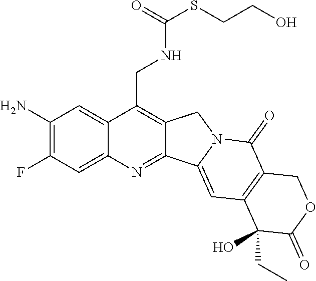 [SMILES: CC[C@@]1(O)C(=O)OCc2c1cc1n(c2=O)Cc2c-1nc1cc(F)c(N)cc1c2CNC(=O)SCCO]

The title compound was prepared according to General Procedure 5 starting from Compound 145 (10 mg) and 2-mercaptoethanol. Preparative HPLC purification was accomplished as described in General Procedure 9, eluting with a 10 to 45% CH₃CN/H₂O+0.1% TFA gradient. The title compound was obtained as a yellow solid (4.2 mg, 43% yield).

LC/MS: Calc'd m/z=514.1 for C₂₄H₂₃FN₄O₆S found [M+H]⁺=515.2.

¹H NMR (300 MHz, MeOD) δ 7.71 (d, J=12.1 Hz, 1H), 7.60 (s, 1H), 7.36 (d, J=9.4 Hz, 1H), 5.62 (d, J=16.3 Hz, 1H), 5.42 (s, 2H), 5.35 (d, J=16.2 Hz, 1H), 4.88 (d, J=4.6 Hz, 2H), 3.68 (t, J=6.4 Hz, 2H), 3.03 (t, J=6.5 Hz, 2H), 2.04-1.92 (m, 2H), 1.03 (t, J=7.4 Hz, 3H).

### 3.28: (S)-9-amino-4,11-diethyl-8-fluoro-4-hydroxy-1,12-dihydro-14H-pyrano[3′,4′:6,7]indolizino[1,2-b]quinoline-3,14(4H)-dione (Compound 154)

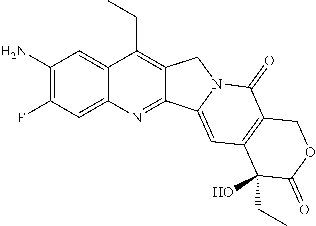 [SMILES: CCc1c2c(nc3cc(F)c(N)cc13)-c1cc3c(c(=O)n1C2)COC(=O)[C@]3(O)CC]

To a 5 mL flask containing Compound 140 (50 mg) was added water (0.72 mL), FeSO₄ (heptahydrate, 11.0 mg) and propionaldehyde (74 μL). The obtained suspension was cooled to −15° C. using an ice brine bath, then sulfuric acid (0.40 mL) was added dropwise. Hydrogen peroxide (95 μL) was then added dropwise. This mixture was stirred at −15° C. for 10 min then allowed to warm up to room temperature and stirred for 2 h. The reaction mixture was diluted with water (30 mL) and the obtained suspension was extracted with DCM (3×30 mL). The organic phase was then evaporated to dryness. Preparative HPLC purification was accomplished as described in General Procedure 9, eluting with a 25 to 70% CH₃CN/H₂O+0.1% TFA gradient to give the title compound as a dark orange solid (2.4 mg, 4.4% yield).

LC/MS: Calc'd m/z=410.1 for C₂₂H₂₀FN₃O₄ found [M+H]⁺=410.2.

¹H NMR (300 MHz, MeOD) δ 7.63 (d, J=12.3 Hz, 1H), 7.55 (s, 1H), 7.36 (d, J=9.4 Hz, 1H), 5.57 (d, J=16.4 Hz, 1H), 5.37 (d, J=16.4 Hz, 1H), 5.21 (s, 2H), 3.13 (q, J=7.7 Hz, 2H), 2.02-1.90 (m, 2H), 1.38 (t, J=7.7 Hz, 3H), 1.01 (t, J=7.3 Hz, 3H).

### 3.29: tert-butyl-(S)-(11-((carbamoyloxy)methyl)-4-ethyl-8-fluoro-4-hydroxy-3,14-dioxo-3,4,12,14-tetrahydro-1H-pyrano[3′,4′:6,7]indolizino[1,2-b]quinolin-9-yl)carbamate (Compound 3.29)

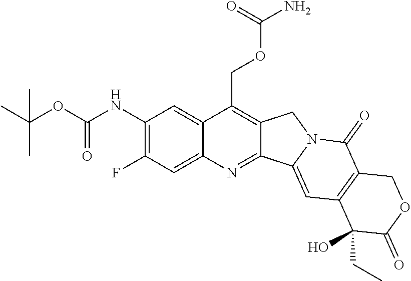 [SMILES: CC[C@@]1(O)C(=O)OCc2c1cc1n(c2=O)Cc2c-1nc1cc(F)c(NC(=O)OC(C)(C)C)cc1c2COC(N)=O]

In a 5 mL conical flask containing a solution of chlorosulfonyl isocyanate (7.7 μL) in dimethylformamide (0.29 mL), at −20° C., was added Compound 3.5 (15 mg). The obtained suspension was stirred at −20° C. for 5 min. Water (59 μL) was added, and the reaction mixture was allowed to warm up to room temperature and stirred for 2 h, then heated at 70° C. for 1 h. The reaction mixture was allowed to cool down to room temperature and partially evaporated. Preparative HPLC purification was accomplished as described in General Procedure 9, eluting with a 40 to 55% CH₃CN/H₂O+0.1% TFA gradient to give the title compound as a dark orange solid (5.1 mg, 31% yield).

LC/MS: Calc'd m/z=555.2 for C₂₇H₂₇FN₄O₈ found [M+H]⁺=555.2.

¹H NMR (300 MHz, DMSO-d6) δ 9.53 (s, 1H), 8.56 (d, J=8.5 Hz, 1H), 8.00 (d, J=12.0 Hz, 1H), 7.31 (s, 1H), 7.11-6.62 (m, 2H), 6.52 (s, 1H), 5.58 (s, 2H), 5.49-5.27 (m, 4H), 1.94-1.77 (m, 2H), 1.52 (s, 9H), 1.38 (t, J=7.7 Hz, 3H), 0.87 (t, J=7.2 Hz, 3H).

### 3.30: (S)-(9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl carbamate (Compound 169)

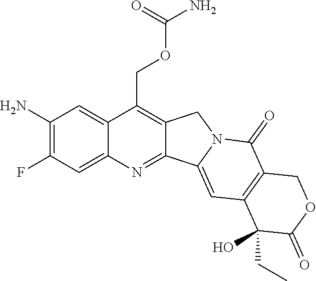 [SMILES: CC[C@@]1(O)C(=O)OCc2c1cc1n(c2=O)Cc2c-1nc1cc(F)c(N)cc1c2COC(N)=O]

The title compound was prepared according to General Procedure 6 starting from Compound 3.29 (5.1 mg) to give the title compound as yellow powder (TFA salt, 3.8 mg, 73% yield).

LC/MS: Calc'd m/z=455.1 for C₂₂H₁₉FN₄O₆ found [M+H]⁺=455.2.

¹H NMR (300 MHz, DMSO-d6) δ 7.79 (d, J=12.4 Hz, 1H), 7.29 (d, J=9.7 Hz, 1H), 7.21 (s, 1H), 7.0-6.50 (m, 2H), 5.45 (s, 2H), 5.40 (s, 2H), 5.33 (s, 2H), 1.95-1.77 (m, 2H), 0.87 (t, J=7.3 Hz, 3H).

### 3.31: ((S)-9-amino-4-ethyl-8-fluoro-4-hydroxy-11-(methoxymethyl)-1,12-dihydro-14H-pyrano[3′,4′:6,7]indolizino[1,2-b]quinoline-3,14(4H)-dione (Compound 155)

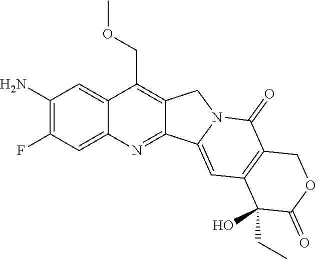 [SMILES: CC[C@@]1(O)C(=O)OCc2c1cc1n(c2=O)Cc2c-1nc1cc(F)c(N)cc1c2COC]

In a 50 mL flask containing Compound 3.5 (30 mg) was added MeOH/Dioxane (1:1) (9.8 mL) and sulfuric acid (0.73 mL). The reaction mixture was then stirred at reflux for 24 h. The reaction mixture was concentrated, poured into water (30 mL), and extracted with DCM (3×50 mL). The organic phases were combined and dried over MgSO₄. Preparative HPLC purification was accomplished as described in General Procedure 9, eluting with a 25 to 40% CH₃CN/H₂O+0.1% TFA gradient to give the title compound as a dark orange solid (5.1 mg, 16% yield).

LC/MS: Calc'd m/z=426.1 for C₂₂H₂₀FN₃O₅ found [M+H]⁺=426.2.

¹H NMR (300 MHz, DMSO-d6) δ 7.75 (d, J=12.3 Hz, 1H), 7.24 (d, J=9.9 Hz, 1H), 7.20 (s, 1H), 6.47 (s, 1H), 6.30-5.92 (brs, 2H), 5.40 (s, 2H), 5.24 (s, 2H), 4.93 (s, 2H), 3.43 (s, 3H), 1.95-1.75 (m, 2H), 0.87 (t, J=7.3 Hz, 3H).

### 3.32: (4S)-9-amino-4-ethyl-8-fluoro-4-hydroxy-11-(((1R,5S)-6-hydroxy-3-azabicyclo[3.1.1]heptan-3-yl)methyl)-1,12-dihydro-14H-pyrano[3′,4′:6,7]indolizino[1,2-b]quinoline-3,14(4H)-dione (Compound 158)

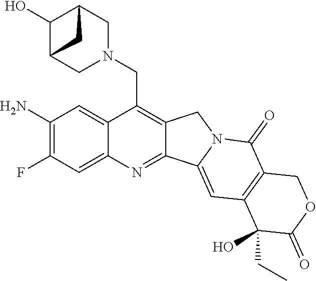 [SMILES: CC[C@@]1(O)C(=O)OCc2c1cc1n(c2=O)Cc2c-1nc1cc(F)c(N)cc1c2CN1C[C@H]2C[C@@H](C1)C2O]

In a 5 mL conical flask containing Compound 3.6 (15 mg) was added dichloromethane (0.6 mL) followed by 3-azabicyclo[3.1.1]heptan-6-ol (10 mg) and acetic acid (7.6 μL). The reaction was stirred at room temperature and sodium triacetoxyborohydride (9.4 mg) was added. After 1 hour at room temperature, the reaction was quenched by addition of water+0.1% TFA and diluted with DMF. The reaction mixture was then partially evaporated. Preparative HPLC purification was accomplished as described in General Procedure 9, eluting with a 20 to 50% CH₃CN/H₂O+0.1% TFA gradient to give the Boc-protected title compound as a yellow powder. Deprotection was performed according to General Procedure 6, and the obtained residue was purified by preparative HPLC purification as described in General Procedure 9, eluting with a 20 to 50% CH₃CN/H₂O+0.1% TFA gradient to give the title compound as yellow powder (TFA salt, 7.1 mg, 39% yield).

LC/MS: Calc'd m/z=507.2 for C₂₇H₂₇FN₄O₅ found [M+H]⁺=507.4.

¹H NMR (300 MHz, DMSO-d6) δ 7.85 (d, J=12.1 Hz, 1H), 7.46 (d, J=9.4 Hz, 1H), 7.23 (s, 1H), 6.64-5.85 (m, 3H), 5.60-5.25 (m, 4H), 4.85 (s, 1H), 4.10-3.95 (m, 1H), 3.68 (s, 2H), 2.45-2.33 (m, 2H), 1.96-1.72 (m, 2H), 0.87 (t, J=7.3 Hz, 3H).

### 3.33: (S)-9-amino-4-ethyl-8-fluoro-11-((3-fluoro-3-(hydroxymethyl)azetidin-1-yl)methyl)-4-hydroxy-1,12-dihydro-14H-pyrano[3′,4′:6,7]indolizino[1,2-b]quinoline-3,14(4H)-dione (Compound 159)

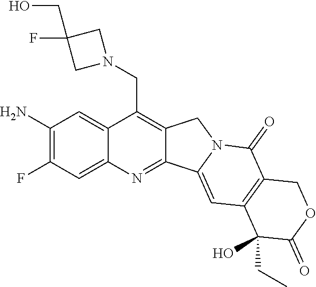 [SMILES: CC[C@@]1(O)C(=O)OCc2c1cc1n(c2=O)Cc2c-1nc1cc(F)c(N)cc1c2CN1CC(F)(CO)C1]

In a 5 mL conical flask containing Compound 3.6 (15 mg) was added dichloromethane (0.6 mL) followed by (3-fluoroazetidin-3-yl)methanol (9.3 mg) and acetic acid (7.6 μL). The reaction was stirred at room temperature and sodium triacetoxyborohydride (9.4 mg) was added. After 1 hour at room temperature, the reaction was quenched by addition of water+0.1% TFA, diluted with DMF, then partially evaporated. Preparative HPLC purification was accomplished as described in General Procedure 9, eluting with a 20 to 50% CH₃CN/H₂O+0.1% TFA gradient to give the Boc-protected title compound as a yellow powder. Deprotection was then performed according to General Procedure 6. The obtained residue was purified by preparative HPLC purification as described in General Procedure 9, eluting with a 20 to 50% CH₃CN/H₂O+0.1% TFA gradient to give the title compound as yellow powder (TFA salt, 1.8 mg, 10% yield).

LC/MS: Calc'd m/z=499.2 for C₂₅H₂₄F₂N₄O₅ found [M+H]⁺=499.4.

¹H NMR (300 MHz, DMSO-d6) δ 7.82 (d, J=12.4 Hz, 1H), 7.45 (d, J=9.5 Hz, 1H), 7.21 (s, 1H), 5.45-5.33 (m, 4H), 3.75-3.61 (m, 2H), 1.93-1.78 (m, 2H), 0.87 (t, J=7.3 Hz, 3H).

### 3.34: tert-butyl-(S)-(4-ethyl-8-fluoro-4-hydroxy-11-((methylamino)methyl)-3,14-dioxo-3,4,12,14-tetrahydro-1H-pyrano[3′,4′:6,7]indolizino[1,2-b]quinolin-9-yl)carbamate (Compound 3.34)

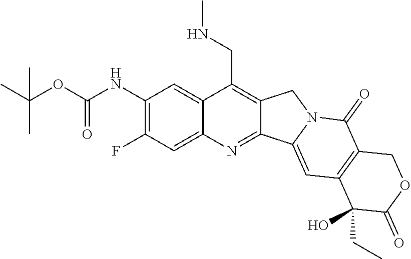 [SMILES: CC[C@@]1(O)C(=O)OCc2c1cc1n(c2=O)Cc2c-1nc1cc(F)c(NC(=O)OC(C)(C)C)cc1c2CNC]

To a stirring solution of Compound 3.9 (210 mg) in DMF (5 mL) was added sodium iodide (5.9 mg) followed by methylammonium chloride (107 mg). The reaction mixture was then stirred at room temperature overnight. Reverse phase purification was accomplished as described in General Procedure 9 using a 30 g C18 column and eluting with a 10 to 65% CH₃CN/H₂O+0.1% TFA gradient to give the title compound as a yellow solid (15.0 mg, 7.2% yield).

LC/MS: Calc'd m/z=524.2 for C₂₇H₂₉FN₄O₆, found [M+H]⁺=525.4.

### 3.35: (S)—N-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-2-hydroxy-N-methylacetamide (Compound 165)

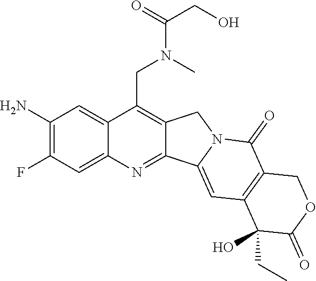 [SMILES: CC[C@@]1(O)C(=O)OCc2c1cc1n(c2=O)Cc2c-1nc1cc(F)c(N)cc1c2CN(C)C(=O)CO]

The Boc-protected version of the title compound was prepared according to General Procedure 2 starting from Compound 3.34 (6.4 mg) and glycolic acid. Preparative HPLC purification was accomplished as described in General Procedure 9, eluting with a 20 to 50% CH₃CN/H₂O+0.1% TFA gradient. Deprotection was then performed according to General Procedure 6 to give the title compound as yellow powder (TFA salt, 2.0 mg, 28% yield).

LC/MS: Calc'd m/z=482.2 for C₂₄H₂₃FN₄O₆, found [M+H]⁺=483.2.

¹H NMR (300 MHz, DMSO-d6) δ 7.79 (d, J=12.3 Hz, 1H), 7.27 (d, J=9.5 Hz, 1H), 7.22 (s, 1H), 6.48 (s, 1H), 6.28-6.02 (m, 2H), 5.40 (s, 2H), 5.21 (s, 2H), 5.06-4.93 (m, 2H), 4.18 (s, 2H), 2.80 (s, 3H), 1.92-1.78 (m, 2H), 0.87 (t, J=7.3 Hz, 3H).

### 3.36: (S)—N-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-N-methylmethanesulfonamide (Compound 166)

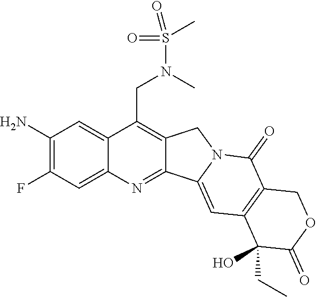 [SMILES: CC[C@@]1(O)C(=O)OCc2c1cc1n(c2=O)Cc2c-1nc1cc(F)c(N)cc1c2CN(C)S(C)(=O)=O]

The Boc-protected version of the title compound was prepared according to General Procedure 3 starting from Compound 3.34 (8.0 mg) and methanesulfonyl chloride. Preparative HPLC purification was accomplished as described in General Procedure 9, eluting with a 10 to 50% CH₃CN/H₂O+0.1% TFA gradient. Deprotection was then performed according to General Procedure 6 to give the title compound as yellow powder (TFA salt, 2.6 mg, 34% yield).

LC/MS: Calc'd m/z=502.1 for C₂₃H₂₃FN₄O₆S, found [M+H]⁺=503.2.

¹H NMR (300 MHz, DMSO-d6) δ 7.81 (d, J=12.3 Hz, 1H), 7.41 (d, J=9.4 Hz, 1H), 7.23 (s, 1H), 6.63-5.84 (m, 2H), 5.42 (s, 2H), 5.29 (s, 2H), 4.81-4.64 (m, 2H), 3.14 (s, 3H), 2.67 (s, 3H), 1.96-1.76 (m, 2H), 0.88 (t, J=7.3 Hz, 3H).

### 3.37: (S)-9-amino-4-ethyl-8-fluoro-4-hydroxy-11-(2-methoxyethyl)-1,12-dihydro-14H-pyrano[3′,4′:6,7]indolizino[1,2-b]quinoline-3,14(4H)-dione (Compound 170)

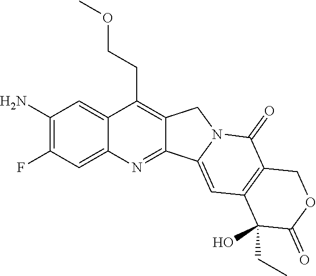 [SMILES: CC[C@@]1(O)C(=O)OCc2c1cc1n(c2=O)Cc2c-1nc1cc(F)c(N)cc1c2CCOC]

To a 10 mL round bottom flask containing Compound 3.4 (62.0 mg) was added water (0.89 mL), FeSO₄ (heptahydrate, 18.0 mg), and 3-methoxypropanal (113.0 mg). To the obtained suspension was added sulfuric acid (0.495 mL) dropwise while stirring at −15° C. in an ice salt bath. Hydrogen peroxide (0.118 mL) was then added dropwise. The mixture was stirred at −15° C. for 10 min and was then allowed to warm up to room temperature and stirred for 1 h. The reaction mixture was then diluted with water (30 mL) and the obtained suspension was extracted with DCM (3×30 mL). The organic phase was evaporated to dryness. Preparative HPLC purification was accomplished as described in General Procedure 9, eluting with a 25 to 45% CH₃CN/H₂O+0.1% TFA gradient to give the title compound as a dark orange solid (TFA salt, 3.1 mg, 4.4% yield).

LC/MS: Calc'd m/z=440.2 for C₂₃H₂₂FN₃O₅, found [M+H]⁺=440.2.

¹H NMR (300 MHz, DMSO-d6) δ 7.75 (d, J=12.4 Hz, 1H), 7.33 (d, J=9.4 Hz, 1H), 7.20 (s, 1H), 6.60-6.42 (m, 2H), 5.40 (s, 2H), 5.25 (s, 2H), 3.69 (t, J=6.5 Hz, 2H), 3.24 (s, 3H), 3.23 (t, J=6.5 Hz, 2H), 1.96-1.76 (m, 2H), 0.88 (t, J=7.3 Hz, 3H).

### 3.38: (S)—N-(4-ethyl-8-fluoro-4-hydroxy-3,14-dioxo-3,4,12,14-tetrahydro-1H-pyrano[3′,4′:6,7]indolizino[1,2-b]quinolin-9-yl)acetamide (Compound 171)

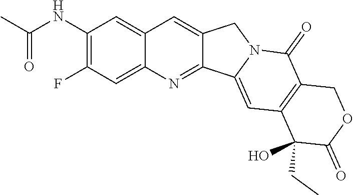 [SMILES: CC[C@@]1(O)C(=O)OCc2c1cc1n(c2=O)Cc2cc3cc(NC(C)=O)c(F)cc3nc2-1]

To a 25 mL round bottom flask containing acetic acid (0.071 mL) in dimethylformamide (0.69 mL) was added N-methylmorpholine (0.343 mL), HOAt (0.142 g), and HATU (0.435 g). After stirring at room temperature for 5 min, this solution was added to a 10 mL cone-shaped flask containing Compound 140 (0.127 g). This solution was stirred at room temperature for 24 h then directly purified by preparative HPLC as described in General Procedure 9, eluting with a 25 to 45% CH₃CN/H₂O+0.1% TFA gradient to give the title compound as a bright yellow powder (43.0 mg, 38% yield).

LC/MS: Calc'd m/z=424.1 for C₂₂H₁₈FN₃O₅, found [M+H]⁺=424.2.

¹H NMR (300 MHz, DMSO-d6) δ 10.13 (s, 1H), 8.73 (d, J=8.5 Hz, 1H), 8.61 (s, 1H), 7.96 (d, J=912.1 Hz, 1H), 7.29 (s, 1H), 6.60-6.42 (m, 2H), 5.41 (s, 2H), 5.21 (s, 2H), 2.20 (s, 3H), 1.96-1.76 (m, 2H), 0.88 (t, J=7.3 Hz, 3H).

### 3.39: tert-butyl (5-formyl-2-methoxy-4-nitrophenyl)carbamate (Compound 3.39)

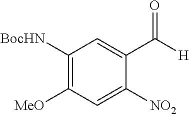 [SMILES: COc1cc([N+](=O)[O-])c(C=O)cc1NC(=O)OC(C)(C)C]

To a solution of Compound 3.2 (1.3 g, 1.0 eq.) in MeOH (12 mL) at 0° C. was added sodium methoxide (0.74 g, 3.0 eq.). After the addition was complete, the ice bath was removed and the resulting solution was stirred at room temperature for 72 h. The reaction was then quenched with ice water (50 mL) and extracted with DCM (3×100 mL). The combined organic layers were washed with brine (50 mL), dried over sodium sulfate, filtered, and concentrated in vacuo to yield the title compound as an orange solid (1.2 g, 89% yield).

LC/MS: Calc'd m/z=296.10 for C₁₃H₁₆N₂O₆, found [M+H]⁺=297.1.

¹H NMR (300 MHz, MeOD) δ 10.29 (s, 1H), 8.61 (s, 1H), 7.73 (s, 1H), 4.08 (s, 3H), 1.57 (s, 9H)

### 3.40: tert-butyl (4-amino-5-formyl-2-methoxyphenyl)carbamate (Compound 3.40)

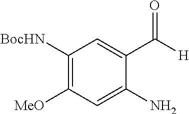 [SMILES: COc1cc(N)c(C=O)cc1NC(=O)OC(C)(C)C]

To a solution of Compound 3.39 (500 mg, 1 eq.) in MeOH (10 mL) and H₂O (1 mL) was added B₂(OH)₄ (454 mg, 3 eq.). The resulting mixture was cooled to 0° C. and an aqueous 5M NaOH solution (2.75 mL) was added with stirring over the course of 10 min. The reaction mixture was stirred for an additional 5 min then quenched by pouring the solution into ice (40 mL). The resulting mixture was extracted with DCM (3×50 mL), dried over sodium sulfate, filtered, and concentrated in vacuo. Flash purification was accomplished as described in General Procedure 9, using a 25 g silica column and eluting with 10 to 50% hexanes/EtOAc to give the title compound as an orange solid (386 mg, 86%).

LC/MS: Calc'd m/z=266.1 for C₁₃H₁₈N₂O₄, found [M+H]⁺=297.2.

### 3.41: (S)-9-amino-4-ethyl-8-fluoro-4-hydroxy-1,12-dihydro-14H-pyrano[3′,4′:6,7]indolizine o[1,2-b]quinoline-3,14(4H)-dione (Compound 168)

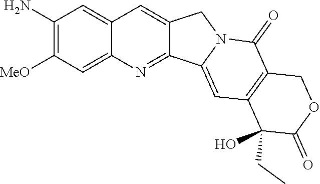 [SMILES: CC[C@@]1(O)C(=O)OCc2c1cc1n(c2=O)Cc2cc3cc(N)c(OC)cc3nc2-1]

A mixture of Compound 3.40 (385 mg, 1.0 eq.) and (S)-4-ethyl-4-hydroxy-7,8-dihydro-1H-pyrano[3,4-f]indolizine-3,6,10(4H)-trione (362 mg, 0.95 eq.), TsOH (monohydrate, 25 mg, 0.1 eq.) and toluene (30 mL) in a 250 mL round bottom flask equipped with a Dean-Stark apparatus was stirred at 110° C. for 2 h. The reaction mixture was then cooled to 25° C. and concentrated in vacuo. Purification was accomplished as described in General Procedure 9, using a 25 g silica column and eluting with a 0 to 50% DCM/MeOH gradient to provide the Boc-protected intermediate as a red solid. This material was then deprotecting according to General Procedure 6 followed by preparative HPLC purification as described in General Procedure 9, eluting with a 20 to 65% CH₃CN/H₂O+0.1% TFA gradient to give the title compound as a red solid (TFA salt, 300 mg, 53% yield).

LC/MS: Calc'd m/z=393.2 for C₂₁H₁₉N₃O₅, found [M+H]⁺=393.2.

¹H NMR (300 MHz, MeOD) δ 8.27 (s, 1H), 7.62 (s, 1H), 7.42 (s, 1H), 7.11 (s, 1H), 5.61 (d, J=16.2 Hz, 1H), 5.38 (d, J=16.2 Hz, 1H), 5.24 (s, 2H), 4.11 (s, 3H), 2.06-1.91 (m, 2H), 1.04 (t, J=7.4 Hz, 3H).

### 3.42: 5-bromo-2-nitro-4-(trifluoromethyl)benzaldehyde (Compound 3.42)

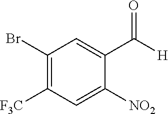 [SMILES: O=Cc1cc(Br)c(C(F)(F)F)cc1[N+](=O)[O-]]

To a stirring solution of HNO₃ (2.0 g, 1.4 mL, 67% purity, 2 eq.) in H₂SO₄ (8 mL) at 0° C. was added 3-bromo-4-(trifluoromethyl)benzaldehyde (4 g, 1 eq.). After the addition was complete, the ice bath was removed, and the reaction was allowed to stir for 5 h at room temperature. The mixture was poured into ice (100 mL) and the precipitate extracted with DCM (3×100 mL). The combined organic fractions were then washed with brine (50 mL), dried over Na₂SO₄, and concentrated in vacuo to yield the title compound as a yellow solid (4.4 g, 93% yield).

LC/MS: Calc'd m/z=296.90 for C₈H₃BrF₃NO₃, found [M+H]⁺=298.0.

¹H NMR (300 MHz, MeOD) δ 10.35 (s, 1H), 8.29 (s, 1H), 8.23 (s, 1H).

### 3.43: tert-butyl (5-formyl-4-nitro-2-(trifluoromethyl)phenyl)carbamate (Compound 3.43)

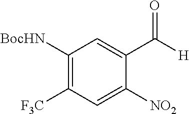 [SMILES: CC(C)(C)OC(=O)Nc1cc(C=O)c([N+](=O)[O-])cc1C(F)(F)F]

A mixture of Compound 3.42 (800 mg, 1 eq.), tert-butyl carbamate (378 mg, 1.2 eq.), Cs₂CO₃ (1.7 g, 2 eq.), Pd₂(dba)₃ (122 mg, 0.05 eq.), and dicyclohexyl[2′,4′,6′-tris(propan-2-yl)[1,1′-biphenyl]-2-yl]phosphane (XPhos) (256 mg, 0.2 eq.) in toluene (5 mL) was degassed and purged with N₂ for three cycles. The mixture was then stirred at 90° C. for 15 h under N₂ atmosphere. The reaction mixture was diluted with H₂O (25 mL) and extracted with EtOAc (3×50 mL). The combined organic layers were washed with brine (2×25 mL), dried over sodium sulfate, filtered, and concentrated under reduced pressure. Flash purification was achieved according to General Procedure 9, using a 25 g silica column and eluting with 0 to 25% DCM/MeOH to give the title compound as an orange solid (750 mg, 84% yield).

LC/MS: Calc'd m/z=334.1 for C₁₃H₁₃FN₂₀₅, found [M−H]⁻=333.1.

### 3.44: tert-butyl (4-amino-5-formyl-2-(trifluoromethyl)phenyl)carbamate (Compound 3.44)

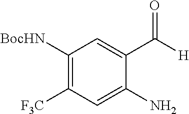 [SMILES: CC(C)(C)OC(=O)Nc1cc(C=O)c(N)cc1C(F)(F)F]

To a solution of Compound 3.43 (750 mg, 1 eq.) in MeOH (16 mL) and H₂O (1.6 mL) was added B₂(OH)₄ (603 mg, 3 eq.). The resulting mixture was cooled to 0° C. and an aqueous 5M NaOH solution (2.75 mL) was added with stirring over the course of 10 min. The reaction mixture was stirred for an additional 5 min then quenched by pouring the solution into ice (50 mL). The resulting mixture was extracted with DCM (3×75 mL), dried over sodium sulfate, filtered, and concentrated in vacuo. Flash purification was accomplished as described in General Procedure 9, using a 25 g silica column and eluting with 10 to 50% hexanes/EtOAc to give the title compound as an orange solid (460 mg, 67%).

LC/MS: Calc'd m/z=304.1 for C₁₃H₁₅F₃N₂₀₃, found [M+H]⁺=305.2

### 3.45: (S)-9-amino-4-ethyl-4-hydroxy-8-(trifluoromethyl)-1,12-dihydro-14H-pyrano[3′,4′:6,7]indolizino [1,2-b]quinoline-3,14(4H)-dione (Compound 167)

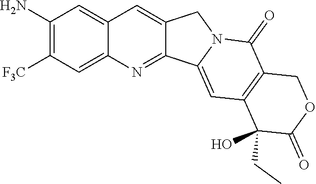 [SMILES: CC[C@@]1(O)C(=O)OCc2c1cc1n(c2=O)Cc2cc3cc(N)c(C(F)(F)F)cc3nc2-1]

A mixture of Compound 3.44 (460 mg, 1 eq.) and (S)-4-ethyl-4-hydroxy-7,8-dihydro-1H-pyrano[3,4-f]indolizine-3,6,10(4H)-trione (378 mg, 0.95 eq.), TsOH (monohydrate, 26 mg, 0.1 eq.) and toluene (35 mL) in a 250 mL round bottom flask equipped with a Dean-Stark apparatus was stirred at 110° C. for 2 h. The reaction mixture was then cooled to 25° C. and concentrated in vacuo. Purification was accomplished as described in General Procedure 9, using a 25 g silica column and eluting with a 0 to 50% DCM/MeOH gradient to provide the Boc-protected intermediate as a red solid. This material was then deprotecting according to General Procedure 6 followed by preparative HPLC purification as described in General Procedure 9, eluting with a 20 to 65% CH₃CN/H₂O+0.1% TFA gradient to give the titled compound as a yellow solid (6.2 mg, 48%).

LC/MS: Calc'd m/z=431.1 for C₂₁H₁₆F₃N₃₀₄, found [M+H]⁺=432.2.

¹H NMR (300 MHz, MeOD) δ 8.29 (s, 1H), 8.27 (s, 1H), 7.59 (s, 1H), 7.24 (s, 1H), 5.59 (d, J=16.3 Hz, 1H), 5.39 (d, J=16.3 Hz, 1H), 5.28 (s, 2H), 2.00-1.89 (m, 2H), 1.03 (t, J=7.4 Hz, 3H).

### Example 4: Preparation of Drug-Linkers

### 4.1: 2,5-dioxopyrrolidin-1-yl (((9H-fluoren-9-yl)methoxy)carbonyl)glycylglycinate (Compound 4.1)

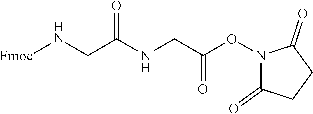

The title compound was prepared according to the procedure described in Chinese Patent Publication No. CN105218644.

### 4.2: (((9H-fluoren-9-yl)methoxy)carbonyl)glycylglycyl-L-phenylalanine (Fmoc-GGF-OH; Compound 4.2)

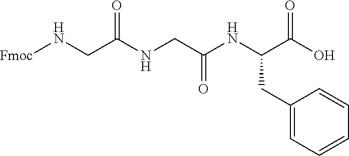

To L-phenylalanine (965 mg) in acetonitrile (10 mL) and dimethyl formamide (0.5 mL) was added DIPEA (1.51 mL) then Compound 4.1 (1.3 g). After 1 h the reaction was concentrated to dryness. Flash purification was accomplished as described in General Procedure 9, eluting with a 10 to 50% CH₃CN/H₂O+0.1% TFA gradient to provide the title compound as a white solid (430 mg, 30% yield).

LC/MS: Calc'd m/z=501.2 for C₂₈H₇₁N₃₀₆S, found [M+H]⁺=502.4.

¹H NMR (300 MHz, DMSO) δ 8.16 (d, J=8.1 Hz, 1H), 8.04 (t, J=5.8 Hz, 1H), 7.90 (d, J=7.5 Hz, 2H), 7.72 (d, J=7.4 Hz, 2H), 7.59 (t, J=6.0 Hz, 1H), 7.54-7.39 (m, 2H), 7.33 (t, J=7.6 Hz, 2H), 7.28-7.13 (m, 5H), 4.44 (td, J=8.5, 5.1 Hz, 1H), 4.33-4.13 (m, 3H), 3.83-3.59 (m, 4H), 3.06 (dd, J=13.7, 5.1 Hz, 1H), 2.88 (dd, J=13.8, 9.0 Hz, 1H).

### 4.3: 2,3,5,6-tetrafluorophenyl 3-(2-(2-(2-(2,5-dioxo-2,5-dihydro-1H-pyrrol-1-yl)ethoxy) ethoxy)ethoxy)propanoate (MT-OTfp; Compound 4.3)

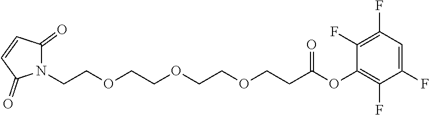 [SMILES: O=C(CCOCCOCCOCCN1C(=O)C=CC1=O)Oc1c(F)c(F)cc(F)c1F]

The title compound was prepared according to the procedure described in International Patent Publication No. WO 2017/054080.

### 4.4: (3-(2-(2-(2-(2,5-dioxo-2,5-dihydro-1H-pyrrol-1-yl)ethoxy)ethoxy)ethoxy)propanoyl) glycylglycyl-L-phenylalanine (Compound 4.4)

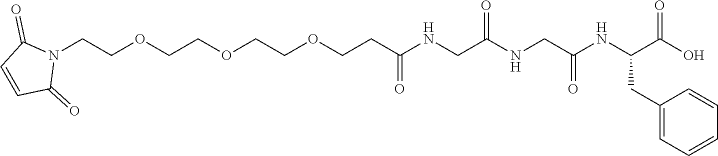 [SMILES: O=C(CCOCCOCCOCCN1C(=O)C=CC1=O)NCC(=O)NCC(=O)N[C@@H](Cc1ccccc1)C(=O)O]

To a solution of Compound 4.3 (1.61 g, 3.58 mmol) in DMF (35 mL) was added Gly-Gly-Phe (1 g, 3.58 mmol) as a single portion followed by iPr₂NEt (1.25 mL, 7.2 mmol). This solution was stirred at room temperature for 1 h, then evaporated to dryness. Purification was accomplished as described in General Procedure 9 using a 30 g C18 flash column and eluting with a 10 to 90% CH₃CN/H₂O+0.1% TFA gradient to provide the title compound as a white solid (400 mg, 20% yield).

LC/MS: Calc'd m/z=562.6 for C₂₆H₃₄N₄₀₁₀, found [M−H]⁻=561.5.

¹H NMR (300 MHz, CDCl₃) δ 7.60 (t, J=5.6 Hz, 2H), 7.41 (d, J=7.7 Hz, 1H), 7.32-7.07 (m, 5H), 6.70 (s, 2H), 6.33-6.07 (m, 3H), 4.72 (td, J=7.6, 5.3 Hz, 1H), 4.12-3.78 (m, 4H), 3.72 (ddd, J=15.2, 6.9, 4.8 Hz, 5H), 3.60 (dd, J=11.6, 6.1 Hz, 10H), 3.12 (ddd, J=48.2, 14.0, 6.5 Hz, 2H), 2.52 (d, J=11.7 Hz, 2H).

### 4.5: (S)-11-benzyl-1-(9H-fluoren-9-yl)-3,6,9,12,15-pentaoxo-2-oxa-4,7,10,13,16-pentaazaheptadecan-17-yl acetate (Compound 4.5)

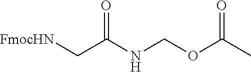

The title compound was prepared according to the procedure described in US Patent Publication No. US 2017/021031.

### 4.6: (S)-11-benzyl-1-(9H-fluoren-9-yl)-3,6,9,12,15-pentaoxo-2-oxa-4,7,10,13,16-pentaazaheptadecan-17-yl acetate (Compound 4.6)

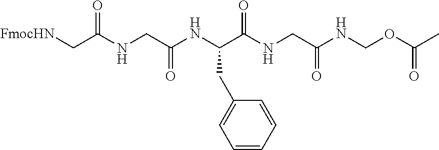

The title compound was prepared according to the procedure described in US Patent Publication No. US 2017/021031 using Fmoc-GGFGG-OH as the starting peptide.

### 4.7. tert-butyl (2-((2-(((S)-1-((2-((4-((4-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)piperazin-1-yl)sulfonyl)phenyl)amino)-2-oxoethyl)amino)-1-oxo-3-phenylpropan-2-yl)amino)-2-oxoethyl)amino)-2-oxoethyl)carbamate (Compound 4.7)

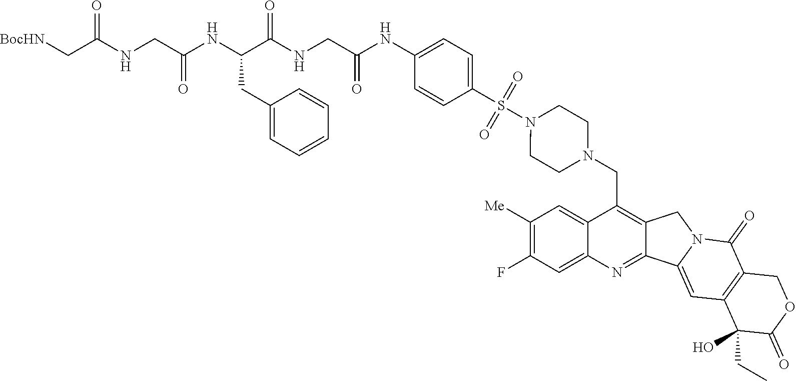 [SMILES: CC[C@@]1(O)C(=O)OCc2c1cc1n(c2=O)Cc2c-1nc1cc(F)c(C)cc1c2CN1CCN(S(=O)(=O)c2ccc(NC(=O)CNC(=O)[C@H](Cc3ccccc3)NC(=O)CNC(=O)CNC(=O)OC(C)(C)C)cc2)CC1]

The title compound was prepared according to General Procedure 7 starting from Compound 104 (20 mg). Preparative HPLC purification was accomplished as described in General Procedure 9, eluting with a 10 to 60% CH₃CN/H₂O+0.1% TFA gradient to provide the title compound as a white solid (14 mg, 42% yield).

LC/MS: Calc'd m/z=1051.4 for C₅₂H₅₈N₉₀₁₂S, found [M+H]⁺=1052.6.

### 4.8: (S)-2-(1-(2,5-dioxo-2,5-dihydro-1H-pyrrol-1-yl)-12,15-dioxo-3,6,9-trioxa-13,16-diazaoctadecan-18-amido)-N-(2-((4-((4-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)piperazin-1-yl)sulfonyl)phenyl)amino)-2-oxoethyl)-3-phenylpropanamide (MT-GGFG-Compound 104)

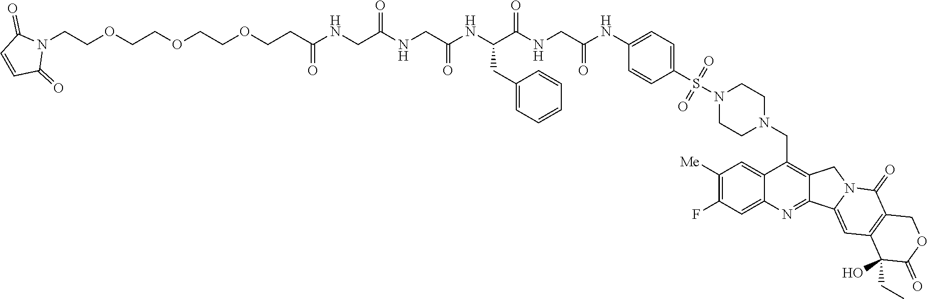 [SMILES: CC[C@@]1(O)C(=O)OCc2c1cc1n(c2=O)Cc2c-1nc1cc(F)c(C)cc1c2CN1CCN(S(=O)(=O)c2ccc(NC(=O)CNC(=O)[C@H](Cc3ccccc3)NC(=O)CNC(=O)CNC(=O)CCOCCOCCOCCN3C(=O)C=CC3=O)cc2)CC1]

The title compound was prepared according to Procedure 6 followed by Procedure 8 starting from Compound 4.7 (14 mg). Preparative HPLC purification was accomplished as described in General Procedure 9, eluting with a 10 to 60% CH₃CN/H₂O+0.1% TFA gradient to provide the title compound as a white solid (9.1 mg, 56% yield).

LC/MS: Calc'd m/z=1234.4 for C₆₀H₆₇FN₁₀O₁₆S, found [M+H]⁺=1235.8.

### 4.9: tert-butyl (2-((2-(((S)-1-((2-((4-(4-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)piperazin-1-yl)phenyl)amino)-2-oxoethyl)amino)-1-oxo-3-phenylpropan-2-yl)amino)-2-oxoethyl)amino)-2-oxoethyl)carbamate (Compound 4.9)

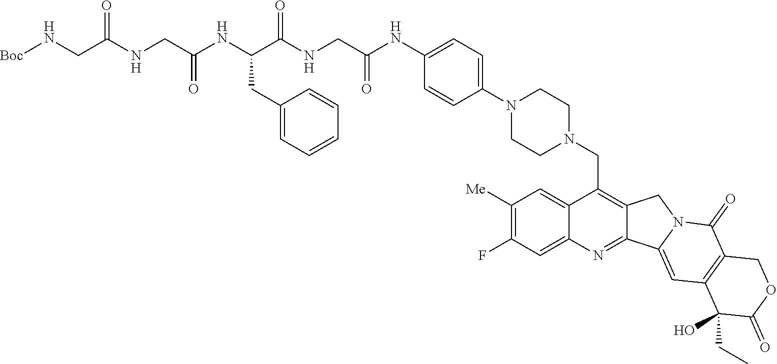 [SMILES: CC[C@@]1(O)C(=O)OCc2c1cc1n(c2=O)Cc2c-1nc1cc(F)c(C)cc1c2CN1CCN(c2ccc(NC(=O)CNC(=O)[C@H](Cc3ccccc3)NC(=O)CNC(=O)CNC(=O)OC(C)(C)C)cc2)CC1]

The title compound was prepared according to General Procedure 7 starting from Compound 108 (12 mg). Preparative HPLC purification was accomplished as described in General Procedure 9, eluting with a 10 to 60% CH₃CN/H₂O+0.1% TFA gradient to provide the title compound as a white solid (13 mg, 62% yield).

LC/MS: Calc'd m/z=987.4 for C₅₂H₅₈N₉O₁₀, found [M+H]⁺=988.6.

### 4.10: (S)-2-(1-(2,5-dioxo-2,5-dihydro-1H-pyrrol-1-yl)-12,15-dioxo-3,6,9-trioxa-13,16-diazaoctadecan-18-amido)-N-(2-((4-(4-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)piperazin-1-yl)phenyl)amino)-2-oxoethyl)-3-phenylpropanamide (MT-GGFG-Compound 108)

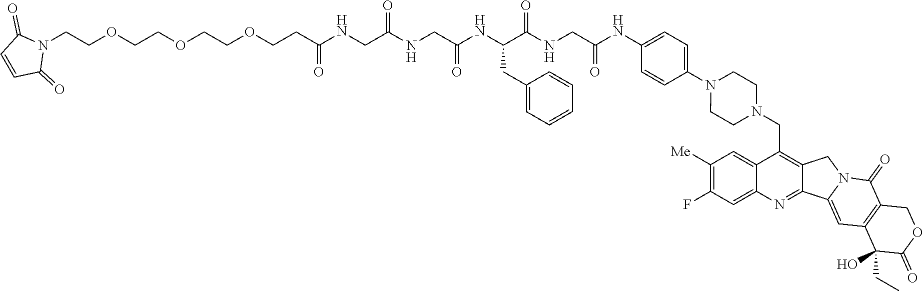 [SMILES: CC[C@@]1(O)C(=O)OCc2c1cc1n(c2=O)Cc2c-1nc1cc(F)c(C)cc1c2CN1CCN(c2ccc(NC(=O)CNC(=O)[C@H](Cc3ccccc3)NC(=O)CNC(=O)CNC(=O)CCOCCOCCOCCN3C(=O)C=CC3=O)cc2)CC1]

The title compound was prepared according to Procedure 6 followed by Procedure 8 starting from Compound 4.9 (13 mg). Preparative HPLC purification was accomplished as described in General Procedure 9, eluting with a 10 to 50% CH₃CN/H₂O+0.1% TFA gradient to provide the title compound as a white solid (3.1 mg, 20% yield).

LC/MS: Calc'd m/z=1170.5 for C₆₀H₆₇FN₁₀O₁₄, found [M+H]⁺=1171.6.

### 4.11: (9H-fluoren-9-yl)methyl (S)-(1-(4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-3,10-dioxo-7-oxa-2,4,9-triazaundecan-11-yl)carbamate (Compound 4.11)

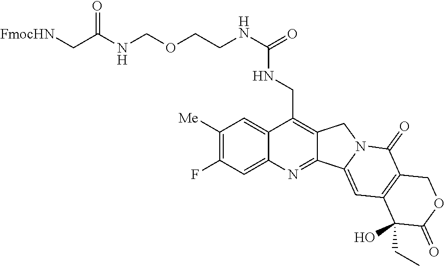

To a solution of Compound 1.2 (31 mg, 0.076 mmol) in DMF (750 μL) was added (9H-fluoren-9-yl)methyl (2-(((2-(((4-nitrophenoxy)carbonyl)amino)ethoxy)methyl)amino)-2-oxoethyl)carbamate (41 mg, 0.076 mmol) followed by iPr₂NEt (26 μL, 0.15 mmol). This solution was stirred at room temperature for 2 h and then applied directly to 12 g C18 column. Purification was accomplished as described in General Procedure 9, eluting with a 10 to 100% CH₃CN/H₂O+0.1% TFA gradient to provide the title compound as a white solid (21 mg, 35% yield).

LC/MS: Calc'd m/z=804.87 for C₄₃H₄₁FN₆₀₉, found [M+H]⁺=805.6.

### 4.12: (S)-2-(1-(2,5-dioxo-2,5-dihydro-1H-pyrrol-1-yl)-12,15-dioxo-3,6,9-trioxa-13,16-diazaoctadecan-18-amido)-N-(1-((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)-3,10-dioxo-7-oxa-2,4,9-triazaundecan-11-yl)-3-phenylpropanamide (MT-GGFG-AM-Compound 136)

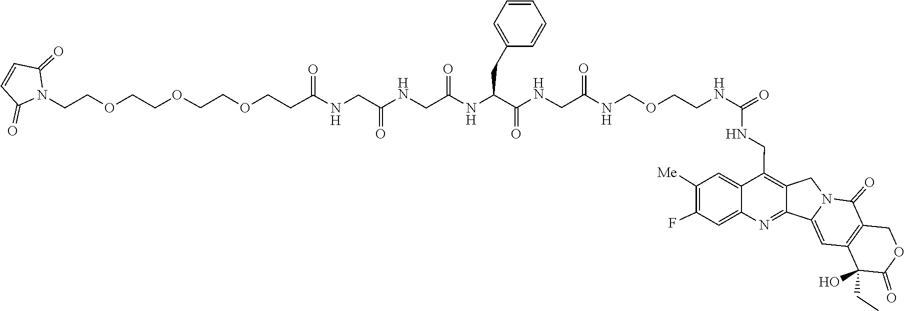 [SMILES: CC[C@@]1(O)C(=O)OCc2c1cc1n(c2=O)Cc2c-1nc1cc(F)c(C)cc1c2CNC(=O)NCCOCNC(=O)CNC(=O)[C@H](Cc1ccccc1)NC(=O)CNC(=O)CNC(=O)CCOCCOCCOCCN1C(=O)C=CC1=O]

Compound 4.11 (21 mg, 0.026 mmol) was taken up in a 10% solution of piperidine in DMF (1 mL) and stirred for 10 min. The piperidine solution was evaporated, the resulting residue was redissolved in DMF (5 mL), and then evaporated to dryness once more. To this residue was added DMF (50 μL) and DCM (450 μL) followed by Compound 4.4 (15 mg, 0.026 mmol), NMM (10 μL) and HATU (10 mg, 0.026 mmol). Preparative HPLC purification was accomplished as described in General Procedure 9, eluting with a 30 to 60% CH₃CN/H₂O+0.1% TFA gradient to provide the title compound as a yellow solid (7.6 mg, 26% yield).

LC/MS: Calc'd m/z=1127.1 for C₅₄H₆₃FN₁₀O₁₆, found [M+H]⁺=1128.2.

### 4.13: (9H-fluoren-9-yl)methyl (2-(((2-(chlorosulfonyl)ethoxy)methyl)amino)-2-oxoethyl)carbamate (Compound 4.13)

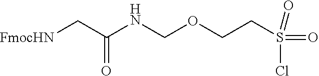

To a solution of Compound 4.5 (50 mg, 0.14 mmol), in DCM (800 μL) was added 2-hydroxyethane-1-sulfonyl chloride (100 mg, 0.7 mmol) followed by TFA (200 μL). This solution was stirred at room temperature for 30 min then evaporated to dryness. Purification was accomplished as described in General Procedure 9, using a 10 g silica column and eluting with a 10 to 100% EtOAc/Hexanes gradient to provide the title compound as a clear film (31 mg, 50% yield).

LC/MS: Calc'd m/z=452.1 for C₂₀H₂₁ClN₂O₆S, found [M+Na]⁺=472.9

### 4.14: (9H-fluoren-9-yl)methyl (S)-(2-(((2-(N-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)sulfamoyl)ethoxy)methyl)amino)-2-oxoethyl)carbamate (Compound 4.14)

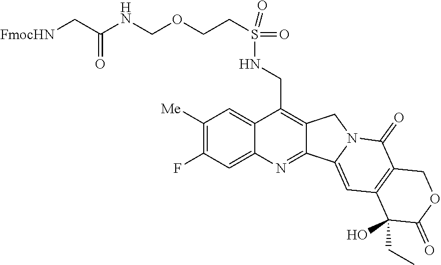

The title compound was prepared as described in General Procedure 3, using Compound 1.2 (28 mg, 0.07 mmol) and Compound 4.13 (31 mg, 0.07 mmol). Purification was accomplished as described in General Procedure 9, using a 12 g C18 column and eluting with a 10 to 100% CH₃CN/H₂O+0.1% TFA gradient to provide the title compound as a yellow solid (22 mg, 39% yield).

LC/MS: Calc'd m/z=825.9 for C₄₂H₄₀FN₅O₁₀S, found [M+H]⁺=826.7.

### 4.15: (S)-2-(1-(2,5-dioxo-2,5-dihydro-1H-pyrrol-1-yl)-12,15-dioxo-3,6,9-trioxa-13,16-diazaoctadecan-18-amido)-N-(2-(((2-(N—(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)sulfamoyl) ethoxy)methyl)amino)-2-oxoethyl)-3-phenylpropanamide (MT-GGFG-AM-Compound 129)

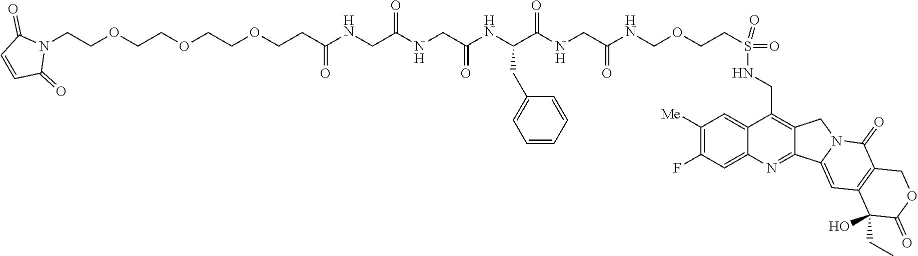 [SMILES: CC[C@@]1(O)C(=O)OCc2c1cc1n(c2=O)Cc2c-1nc1cc(F)c(C)cc1c2CNS(=O)(=O)CCOCNC(=O)CNC(=O)[C@H](Cc1ccccc1)NC(=O)CNC(=O)CNC(=O)CCOCCOCCOCCN1C(=O)C=CC1=O]

Compound 4.14 (22 mg, 0.027 mmol) was taken up in a 10% solution of piperidine in DMF (1 mL) and stirred for 10 min. The piperidine solution was evaporated, the resulting residue was redissolved in DMF (5 mL), and then evaporated to dryness once more. To this residue was added DMF (50 μL) and DCM (450 μL) followed by Compound 4.4 (30 mg, 0.053 mmol), NMM (10 μL) and HATU (18 mg, 0.048 mmol). Preparative HPLC purification was accomplished as described in General Procedure 9, eluting with a 30 to 60% CH₃CN/H₂O+0.1% TFA gradient to provide the title compound as a yellow solid (6.4 mg, 21% yield).

LC/MS: Calc'd m/z=1148.2 for C₅₃H₆₂FN₉O₁₇S, found [M+H]⁺=1148.6.

¹H NMR (300 MHz, MeOD) δ 8.60 (t, J=6.5 Hz, 1H), 8.36 (t, J=8.6 Hz, 2H), 8.13 (d, J=6.6 Hz, 1H), 7.77 (d, J=10.6 Hz, 1H), 7.65 (d, J=4.8 Hz, 1H), 7.28-7.00 (m, 6H), 6.80 (s, 2H), 5.69-5.50 (m, 3H), 5.45-5.33 (m, 2H), 4.44 (dd, J=8.7, 5.7 Hz, 1H), 3.96 (t, J=5.3 Hz, 2H), 3.90-3.76 (m, 5H), 3.76-3.57 (m, 7H), 3.09-2.81 (m, 3H), 2.61-2.45 (m, 5H), 2.04-1.90 (m, 2H), 1.03 (t, J=7.3 Hz, 3H).

### 4.16: (9H-fluoren-9-yl)methyl (S)-(2-(((morpholin-2-ylmethoxy)methyl)amino)-2-oxoethyl)carbamate (Compound 4.16)

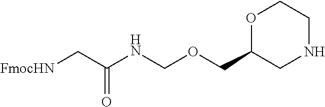

To a solution of Compound 4.5 (100 mg, 0.27 mmol) in DCM (800 μL) was added (S)-morpholin-2-ylmethanol (160 mg, 1.36 mmol) followed by TFA (200 μL). This solution was stirred at room temperature for 1 h then evaporated to dryness. Purification was accomplished as described in General Procedure 9, using a 12 g C18 flash column and eluting with a 10 to 90% CH₃CN/H₂O+0.1% TFA gradient to provide the title compound as a yellow solid (TFA salt, 105 mg, 72% yield).

LC/MS: Calc'd m/z=425.2 for C₂₃H₂₇N₃O₅, found [M+Na]⁺=448.0.

### 4.17. (9H-fluoren-9-yl)methyl (2-(((((S)-4-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)morpholin-2-yl)methoxy)methyl)amino)-2-oxoethyl)carbamate (Compound 4.17)

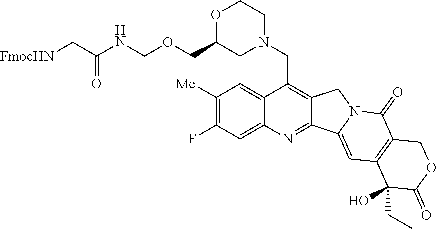

The title compound was prepared according to General Procedure 1 starting from Compound 1.1 (50 mg, 0.117 mmol) and Compound 4.16 (63 mg, 0.117 mmol). Preparative HPLC purification was accomplished as described in General Procedure 9, eluting with a 10 to 100% CH₃CN/H₂O+0.1% TFA gradient to give the title compound as an off-white solid (TFA salt, 33 mg, 35% yield).

LC/MS: Calc'd m/z=817.9 for C₄₅H₄₄FN₅₀₉, found [M+H]⁺=818.7.

### 4.18: (S)-2-(1-(2,5-dioxo-2,5-dihydro-1H-pyrrol-1-yl)-12,15-dioxo-3,6,9-trioxa-13,16-diazaoctadecan-18-amido)-N-(2-(((((S)-4-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)morpholin-2-yl)methoxy)methyl)amino)-2-oxoethyl)-3-phenylpropanamide (MT-GGFG-AM-Compound 113)

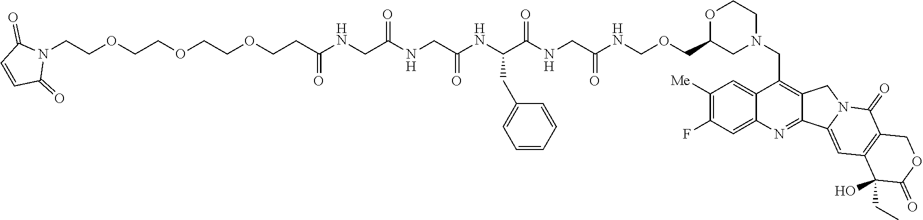 [SMILES: CC[C@@]1(O)C(=O)OCc2c1cc1n(c2=O)Cc2c-1nc1cc(F)c(C)cc1c2CN1CCO[C@H](COCNC(=O)CNC(=O)[C@H](Cc2ccccc2)NC(=O)CNC(=O)CNC(=O)CCOCCOCCOCCN2C(=O)C=CC2=O)C1]

Compound 4.17 (33 mg, 0.04 mmol) was taken up in a 10% solution of piperidine in DMF (1 mL) and stirred for 10 min. The piperidine solution was evaporated, the resulting residue was redissolved in DMF (5 mL), and then evaporated to dryness once more. To this residue was added DMF (100 μL) and DCM (900 μL) followed by Compound 4.4 (45 mg, 0.08 mmol), NMM (20 μL) and HATU (28 mg, 0.073 mmol). Preparative HPLC purification was accomplished as described in General Procedure 9, eluting with a 30 to 60% CH₃CN/H₂O+0.1% TFA gradient to provide the title compound as a yellow solid (22 mg, 48% yield).

LC/MS: Calc'd m/z=1140.2 for C₅₆H₆₆FN₉O₁₆, found [M+H]⁺=1141.1.

¹H NMR (300 MHz, MeOD) δ 8.35 (d, J=7.5 Hz, 2H), 7.74-7.61 (m, 1H), 7.53 (s, 1H), 7.34-7.10 (m, 6H), 6.81 (s, 2H), 5.65-5.30 (m, 4H), 4.64 (t, J=3.4 Hz, 2H), 4.42 (tt, J=6.3, 2.5 Hz, 1H), 4.09 (d, J=12.3 Hz, 1H), 3.98-3.76 (m, 8H), 3.72 (t, J=6.0 Hz, 2H), 3.69-3.44 (m, 17H), 3.21-2.85 (m, 3H), 2.64-2.42 (m, 5H), 2.03-1.84 (m, 2H), 0.98 (t, J=7.3 Hz, 3H). 4.19: (9H-fluoren-9-yl)methyl (S)-(2-((((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methoxy)methyl) amino)-2-oxoethyl)carbamate (Compound 4.19)

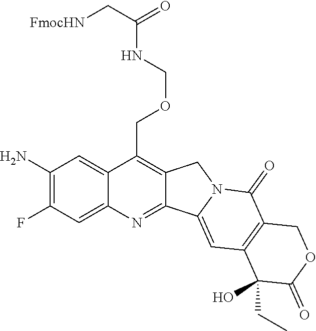

Compound 3.5 (55 mg, 0.11 mmol) was dissolved in TFA (500 μL) and stirred at room temperature for 20 min, then hexafluoroisopropanol (2 mL) was added followed by Compound 4.5 (40 mg, 0.11 mmol). This solution was stirred at room temperature for ˜16 h then concentrated to dryness. Purification was accomplished as described in General Procedure 9, using a 12 g C18 flash column and eluting with a 10 to 50% CH₃CN/H₂O+0.1% TFA gradient to provide the title compound as a yellow solid (11 mg, 14% yield).

LC/MS: Calc'd m/z=719.7 for C₃₉H₃₄FN₅₀₈, found [M+H]⁺=720.6.

### 4.20: (S)—N-(2-(((((S)-9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methoxy)methyl)amino)-2-oxoethyl)-2-(1-(2,5-dioxo-2,5-dihydro-1H-pyrrol-1-yl)-12,15-dioxo-3,6,9-trioxa-13,16-diazaoctadecan-18-amido)-3-phenylpropanamide (MT-GGFG-AM-Compound 141)

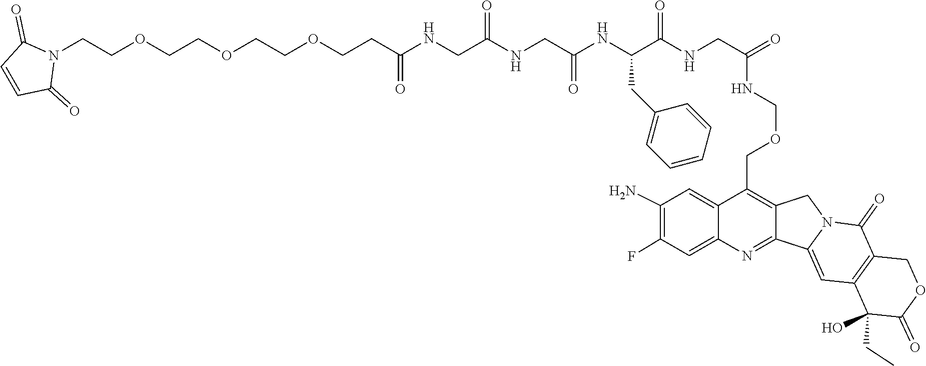 [SMILES: CC[C@@]1(O)C(=O)OCc2c1cc1n(c2=O)Cc2c-1nc1cc(F)c(N)cc1c2COCNC(=O)CNC(=O)[C@H](Cc1ccccc1)NC(=O)CNC(=O)CNC(=O)CCOCCOCCOCCN1C(=O)C=CC1=O]

Compound 4.19 (11 mg, 0.015 mmol) was taken up in a 10% solution of piperidine in DMF (1 mL) and stirred for 10 min. The piperidine solution was evaporated, the resulting residue was redissolved in DMF (5 mL), and then evaporated to dryness once more. To this residue was added DMF (50 μL) and DCM (450 μL) followed by Compound 4.4 (26 mg, 0.045 mmol), NMM (5 μL) and HATU (18 mg, 0.045 mmol). Preparative HPLC purification was accomplished as described in General Procedure 9, eluting with a 32 to 45% CH₃CN/H₂O+0.1% TFA gradient to provide the title compound as a yellow solid (4.6 mg, 29% yield).

LC/MS: Calc'd m/z=1142.0 for C₅₀H₅₆FN₉O₁₅, found [M+H]⁺=1143.1.

¹H NMR (300 MHz, MeOD) δ 8.36 (s, 1H), 8.28 (d, J=6.1 Hz, 1H), 8.16 (dd, J=20.1, 6.8 Hz, 3H), 7.59-7.44 (m, 2H), 7.31-7.08 (m, 6H), 6.79 (s, 2H), 5.58 (d, J=16.1 Hz, 1H), 5.37 (d, J=16.1 Hz, 1H), 5.30-5.16 (m, 3H), 4.56-4.39 (m, 1H), 4.07-3.90 (m, 2H), 3.85 (dt, J=11.5, 5.4 Hz, 4H), 3.79-3.67 (m, 4H), 3.67-3.55 (m, 7H), 3.54 (d, J=6.5 Hz, 8H), 3.10 (dd, J=14.0, 6.1 Hz, 1H), 2.92 (dd, J=13.9, 9.1 Hz, 1H), 2.53 (t, J=6.0 Hz, 2H), 1.98 (q, J=7.2 Hz, 2H), 1.31 (s, 1H), 1.04 (t, J=7.3 Hz, 3H). 4.21: N—((S)-1-((S)-9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)-9-benzyl-5,8,11,14-tetraoxo-2-oxa-4,7,10,13-tetraazapentadecan-15-yl)-6-(2,5-dioxo-2,5-dihydro-1H-pyrrol-1-yl)hexanamide (MC-GGFG-AM-Compound 141)

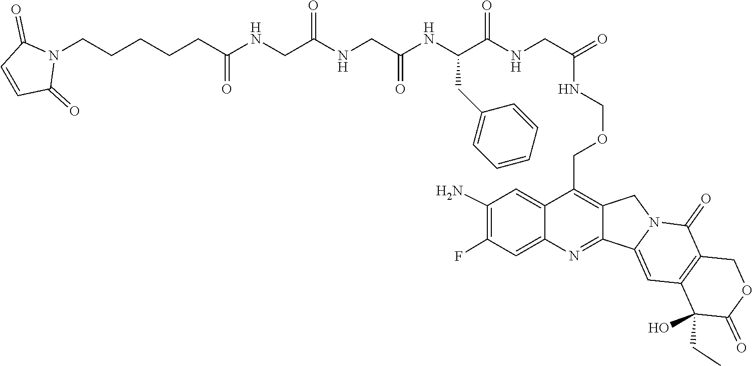 [SMILES: CC[C@@]1(O)C(=O)OCc2c1cc1n(c2=O)Cc2c-1nc1cc(F)c(N)cc1c2COCNC(=O)CNC(=O)[C@H](Cc1ccccc1)NC(=O)CNC(=O)CNC(=O)CCCCCN1C(=O)C=CC1=O]

Compound 4.19 (25 mg, 0.035 mmol) was taken up in a 10% solution of piperidine in DMF (1 mL) and stirred for 10 min. The piperidine solution was evaporated, the resulting residue was redissolved in DMF (5 mL), and then evaporated to dryness once more. To this residue was added DMF (50 μL) and DCM (450 μL), followed by MC-GGF-OH (33 mg, 0.07 mmol), NMM (20 μL) and HATU (25 mg, 0.066 mmol). Preparative HPLC purification was accomplished as described in General Procedure 9, eluting with a 30 to 50% CH₃CN/H₂O+0.1% TFA gradient to provide the title compound as a yellow solid (4.3 mg, 13% yield).

LC/MS: Calc'd m/z=952.0 for C₄₇H₅₀FN₉O₁₂, found [M+H]⁺=952.9.

¹H NMR (300 MHz, CD₃CN) δ 7.96-7.72 (m, 1H), 7.39-7.07 (m, 8H), 6.94 (d, J=9.1 Hz, 1H), 6.73 (s, 2H), 5.44 (d, J=16.2 Hz, 1H), 5.25 (d, J=16.2 Hz, 1H), 5.06 (d, J=4.4 Hz, 2H), 4.81 (d, J=26.1 Hz, 4H), 4.61 (s, 1H), 3.96 (s, 1H), 3.77 (d, J=8.1 Hz, 7H), 3.02 (d, J=5.6 Hz, 5H), 2.19 (t, J=7.7 Hz, 3H), 1.50 (dp, J=14.8, 7.4 Hz, 6H), 1.32-1.12 (m, 3H), 0.96 (t, J=7.2 Hz, 3H). 4.22: tert-butyl (2-((2-(((S)-1-((2-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-2-oxoethyl)amino)-1-oxo-3-phenylpropan-2-yl)amino)-2-oxoethyl)amino)-2-oxoethyl)carbamate (Compound 4.22)

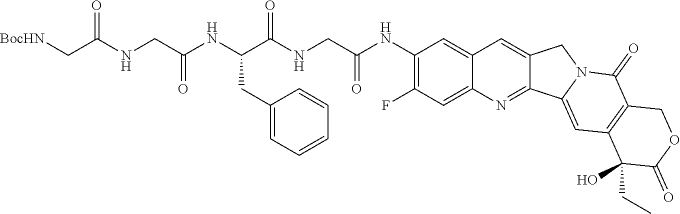 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)OC(C)(C)C)c(F)cc3nc2-1]

The title compound was prepared according to Procedure 7 starting from Compound 140 (28 mg). Preparative HPLC purification was accomplished as described in General Procedure 9, eluting with a 10 to 50% CH₃CN/H₂O+0.1% TFA gradient to provide the title compound as a white solid (10 mg, 17% yield).

LC/MS: Calc'd m/z=799.3 for C₄₀H₄₂N₇₀₁₀, found [M+H]⁺=800.6.

### 4.23: (S)-2-(1-(2,5-dioxo-2,5-dihydro-1H-pyrrol-1-yl)-12,15-dioxo-3,6,9-trioxa-13,16-diazaoctadecan-18-amido)-N-(2-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-2-oxoethyl)-3-phenylpropanamide (MT-GGFG-Compound 140)

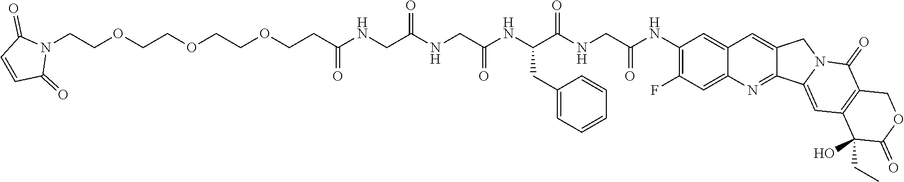 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)CCOCCOCCOCCN4C(=O)C=CC4=O)c(F)cc3nc2-1]

The title compound was prepared according to General Procedure 6 followed by General Procedure 8 starting from Compound 4.22 (10 mg). Preparative HPLC purification was accomplished as described in General Procedure 9, eluting with a 10 to 50% CH₃CN/H₂O+0.1% TFA gradient to provide the title compound as a white solid (6.8 mg, 55% yield).

LC/MS: Calc'd m/z=982.4 for C₄₈H₅₁FN₈O₁₄, found [M+H]⁺=983.6.

### 4.24: tert-butyl (2-((2-(((S)-1-((2-(((S)-4-ethyl-8-fluoro-4-hydroxy-11-(morpholinomethyl)-3,14-dioxo-3,4,12,14-tetrahydro-1H-pyrano[3′,4′:6,7]indolizino[1,2-b]quinolin-9-yl)amino)-2-oxoethyl)amino)-1-oxo-3-phenylpropan-2-yl)amino)-2-oxoethyl)amino)-2-oxoethyl)carbamate (Compound 4.24)

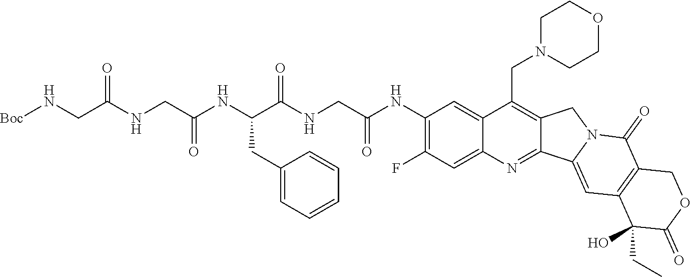 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)OC(C)(C)C)cc1c2CN1CCOCC1]

The title compound was prepared according to General Procedure 7 starting from Compound 142 (TFA salt, 45 mg). Purification was accomplished as described in General Procedure 9, using a 12 g C18 flash column and eluting with a 10 to 50% CH₃CN/H₂O+0.1% TFA gradient to provide the title compound as a white solid (13 mg, 22% yield).

LC/MS: Calc'd m/z=898.4 for C₄₅H₅₁N₈₀₁₁, found [M+H]⁺=899.6.

### 4.25: (S)-2-(1-(2,5-dioxo-2,5-dihydro-1H-pyrrol-1-yl)-12,15-dioxo-3,6,9-trioxa-13,16-diazaoctadecan-18-amido)-N-(2-(((S)-4-ethyl-8-fluoro-4-hydroxy-11-(morpholinomethyl)-3,14-dioxo-3,4,12,14-tetrahydro-1H-pyrano[3′,4′:6,7]indolizino[1,2-b]quinolin-9-yl)amino)-2-oxoethyl)-3-phenylpropanamide (MT-GGFG-Compound 142)

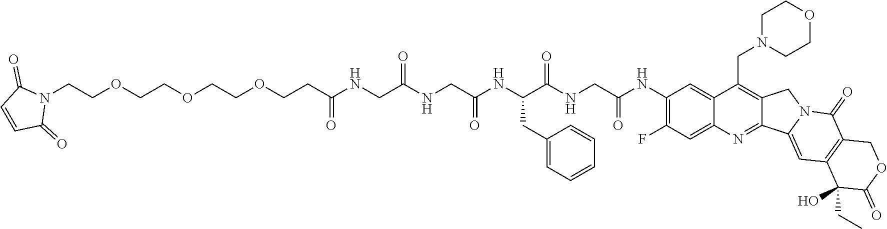 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN1CCOCC1]

The title compound was prepared according to General Procedure 6 followed by General Procedure 8 starting from Compound 4.24 (13 mg). Preparative HPLC purification was accomplished as described in General Procedure 9, eluting with a 10 to 50% CH₃CN/H₂O+0.1% TFA gradient to provide the title compound as a white solid (2.6 mg, 17% yield).

LC/MS: Calc'd m/z=1081.4 for C₅₃H₆₀FN₉O₁₅, found [M+H]⁺=1082.6.

¹H NMR (300 MHz, MeOD) δ 9.34 (d, J=8.5 Hz, 1H), 7.87 (d, J=11.8 Hz, 1H), 7.62 (s, 1H), 7.33-7.19 (m, 5H), 6.80 (s, 2H), 5.62 (d, J=16.3 Hz, 1H), 5.51 (s, 2H), 5.47-5.35 (m, 3H), 4.73 (dd, J=9.6, 5.1 Hz, 1H), 4.61 (s, 3H), 4.30-4.15 (m, 2H), 4.11 (s, 2H), 4.00-3.82 (m, 4H), 3.82-3.70 (m, 7H), 3.70-3.50 (m, 13H), 3.18-3.04 (m, 1H), 2.88 (s, 1H), 2.64 (d, J=5.8 Hz, 4H), 2.54 (t, J=6.0 Hz, 2H), 2.09-1.92 (m, 2H), 1.03 (t, J=7.3 Hz, 3H). 4.26: (9H-fluoren-9-yl)methyl (S)-(12-benzyl-1-(4-nitrophenoxy)-1,8,11,14,17-pentaoxo-2,5-dioxa-7,10,13,16-tetraazaoctadecan-18-yl)carbamate (Compound 4.26)

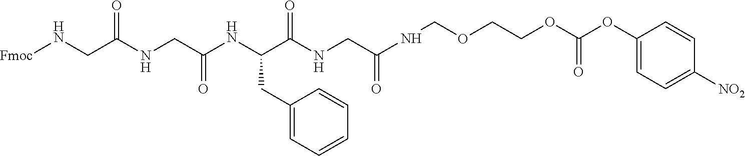

To a stirring solution of Compound 4.6 (60 mg) in dichloromethane (2 mL) was added ethylene glycol (100 μL) followed by trifluoracetic acid (0.4 mL). After 30 min the reaction was concentrated in vacuo. Purification of the intermediate compound was accomplished as described in General Procedure 9, using a 10 g flash column and eluting with a 0 to 20% dichloromethane/methanol gradient. To the purified intermediate in tetrahydrofuran (0.5 mL) was added bis-nitrophenol carbonate (58 mg) followed by DIPEA (50 μL). The solution was stirred for 16 h, quenched with acetic acid (˜100 μL) then concentrated to dryness. Purification was accomplished as described in General Procedure 9, using a 10 g flash column and eluting with a 0 to 20% dichloromethane/methanol gradient to provide the title compound as a white solid (40 mg, 53% yield from Compound 4.6).

LC/MS: Calc'd m/z=796.3 for C₄₀H₄₀N₆O₁₂, found [M+Na]⁺=819.4.

### 4.27: (S)-16-amino-10-benzyl-6,9,12,15-tetraoxo-3-oxa-5,8,11,14-tetraazahexadecyl (((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)carbamate (Compound 4.27)

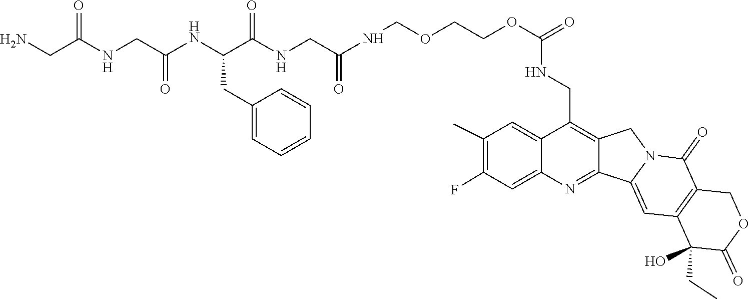 [SMILES: CC[C@@]1(O)C(=O)OCc2c1cc1n(c2=O)Cc2c-1nc1cc(F)c(C)cc1c2CNC(=O)OCCOCNC(=O)CNC(=O)[C@H](Cc1ccccc1)NC(=O)CNC(=O)CN]

To a solution of Compound 4.26 (40 mg) in dimethylformamide (1 mL) was added DIPEA (26 μL) then a solution of Compound 1.2 (24 mg) in dimethylformamide (0.5 mL). This solution was stirred for 4 h at room temperature then quenched with a 20% piperidine in dimethylformamide solution (0.5 mL) and stirred for an additional 20 min. Purification was accomplished as described in General Procedure 9, using a 12 g C18 flash column and eluting with a 10 to 50% CH₃CN/H₂O+0.1% TFA gradient to provide the title compound as a white solid (TFA salt, 19 mg, 39% yield).

LC/MS: Calc'd m/z=844.3 for C₄₁H₄₅FN₈O₁₁, found [M+H]⁺=845.6.

### 4.28: (S)-10-benzyl-29-(2,5-dioxo-2,5-dihydro-1H-pyrrol-1-yl)-6,9,12,15,18-pentaoxo-3,21,24,27-tetraoxa-5,8,11,14,17-pentaazanonacosyl (((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)carbamate (MT-GGFG-AM-Compound 139)

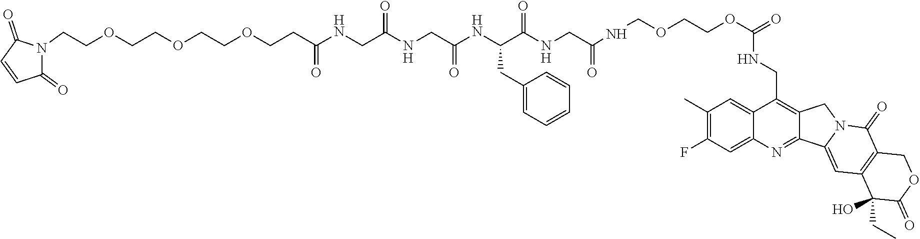 [SMILES: CC[C@@]1(O)C(=O)OCc2c1cc1n(c2=O)Cc2c-1nc1cc(F)c(C)cc1c2CNC(=O)OCCOCNC(=O)CNC(=O)[C@H](Cc1ccccc1)NC(=O)CNC(=O)CNC(=O)CCOCCOCCOCCN1C(=O)C=CC1=O]

The title compound was prepared according to General Procedure 8 starting from Compound 4.27 (10 mg). Preparative HPLC purification was accomplished as described in General Procedure 9, eluting with a 10 to 50% CH₃CN/H₂O+0.1% TFA gradient to provide the title compound as a white solid (8.8 mg, 75% yield).

LC/MS: Calc'd m/z=1127.4 for C₅₄H₆₂FN₉O₁₇, found [M+H]⁺=1128.6.

¹H NMR (300 MHz, MeOD) δ 8.27 (d, J=8.1 Hz, 1H), 7.81 (d, J=10.7 Hz, 1H), 7.65 (s, 1H), 7.32-7.16 (m, 5H), 6.81 (s, 2H), 5.62 (d, J=16.4 Hz, 1H), 5.53 (s, 2H), 5.42 (d, J=16.4 Hz, 1H), 4.93 (s, 2H), 4.67 (s, 1H), 4.51 (dd, J=9.3, 5.6 Hz, 1H), 4.18 (t, J=4.7 Hz, 2H), 4.01-3.44 (m, 19H), 3.17 (dd, J=13.9, 5.8 Hz, 1H), 2.97 (dd, J=13.9, 9.0 Hz, 1H), 2.57 (s, 3H), 2.52 (t, J=6.0 Hz, 2H), 2.03-1.91 (m, 2H), 1.03 (t, J=7.4 Hz, 3H). 4.29: (S)-2-amino-N-(4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)acetamide (Compound 4.29)

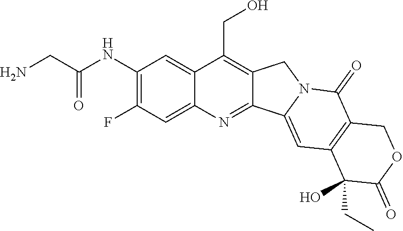 [SMILES: CC[C@@]1(O)C(=O)OCc2c1cc1n(c2=O)Cc2c-1nc1cc(F)c(NC(=O)CN)cc1c2CO]

To a stirring solution of Fmoc-glycine (217 mg) in dimethylformamide (2.5 mL) was added HATU (254 mg), HOAt (83 mg) then NMM (188 μL). This solution was stirred for 10 min then Compound 141 (50 mg) was added and the reaction was stirred at room temperature for 16 h. Lithium hydroxide (2.5 mL, 1 M in water) was added, and the reaction mixture was stirred for 2 h. This solution was partially concentrated, then a solution of 20% piperidine in dimethylformamide (0.5 mL) was added and was stirred for another 20 min. The reaction was then evaporated onto celite and purification was accomplished as described in General Procedure 9, using a 12 g C18 flash column and eluting with a 0 to 40% CH₃CN/H₂O+0.1% TFA gradient to provide the title compound as a white solid (TFA salt, 44 mg, 62% yield).

LC/MS: Calc'd m/z=468.1 for C₂₃H₂₁FN₄O₆, found [M+H]⁺=469.4.

¹H NMR (300 MHz, MeOD) δ 8.99 (d, J=8.3 Hz, 1H), 7.99 (s, 1H), 7.87 (d, J=12.0 Hz, 1H), 7.55 (s, 1H), 5.60 (d, J=16.3 Hz, 1H), 5.46-5.35 (m, 3H), 5.30 (s, 2H), 3.53-3.45 (m, 1H), 3.43-3.38 (m, 1H), 2.03-1.87 (m, 2H), 1.02 (t, J=7.3 Hz, 3H). 4.30: (S)-2-(1-(2,5-dioxo-2,5-dihydro-1H-pyrrol-1-yl)-12,15-dioxo-3,6,9-trioxa-13,16-diazaoctadecan-18-amido)-N-(2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)-3-phenylpropanamide (MT-GGFG-Compound 141)

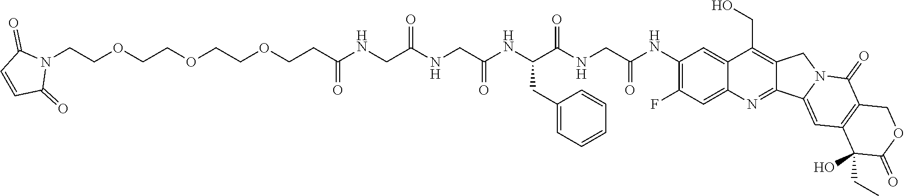 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CO]

To a stirring solution of Compound 4.4 (23 mg) in a mixture of dimethylformamide (0.1 mL) and dichloromethane (0.9 mL) was added HATU (14 mg), a solution of Compound 4.29 (20 mg) in dimethyl formamide (0.1 mL) and dichloromethane (0.9 mL), and DIPEA (24 μL). The mixture was stirred for 15 min, then the reaction was partially concentrated. Preparative HPLC purification was accomplished as described in General Procedure 9, eluting with a 0 to 40% CH₃CN/H₂O+0.1% TFA gradient to provide the title compound as a white solid (7.1 mg, 20% yield).

LC/MS: Calc'd m/z=1012.4 for C₄₉H₅₃FN₈O₁₅, found [M+H]⁺=1013.6.

¹H NMR (300 MHz, MeOD) δ 9.89 (s, 1H), 8.75 (d, J=8.3 Hz, 1H), 8.44-8.32 (m, 1H), 8.27-8.14 (m, 2H), 7.78 (d, J=11.9 Hz, 1H), 7.53 (s, 1H), 7.39-7.20 (m, 5H), 6.82 (s, 2H), 5.57 (d, J=16.3 Hz, 1H), 5.39 (d, J=16.3 Hz, 1H), 5.34-5.25 (m, 2H), 5.22 (s, 2H), 4.32-4.09 (m, 2H), 3.96-3.83 (m, 3H), 3.76 (t, J=6.0 Hz, 2H), 3.69-3.62 (m, 2H), 3.62-3.47 (m, 9H), 3.40-3.33 (m, 1H), 3.08 (dd, J=14.0, 9.6 Hz, 1H), 2.56 (t, J=6.1 Hz, 2H), 2.03-1.91 (m, 2H), 1.04 (t, J=7.3 Hz, 3H). 4.31: tert-butyl (S)-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)carbamate (Compound 4.31)

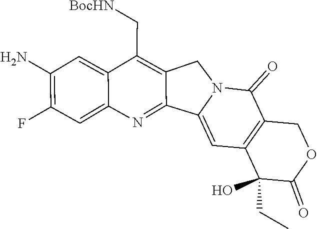 [SMILES: CC[C@@]1(O)C(=O)OCc2c1cc1n(c2=O)Cc2c-1nc1cc(F)c(N)cc1c2CNC(=O)OC(C)(C)C]

To a stirring solution of Compound 145 (32 mg) in dichloromethane (2 mL) and acetonitrile (0.5 mL) was added di-tert-butyl dicarbonate (20 μL) followed by DIPEA (42 μL). The reaction mixture was stirred at room temperature for 3 h then concentrated to dryness to provide the title compound as a red solid (34 mg, 87%).

LC/MS: Calc'd m/z=510.2 for C₂₆H₂₇FN₄O₆, found [M+H]⁺=511.2.

### 4.32: tert-butyl (S)-((9-(2-aminoacetamido)-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)carbamate (Compound 4.32)

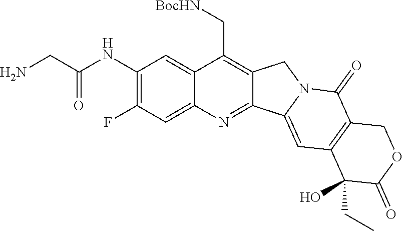 [SMILES: CC[C@@]1(O)C(=O)OCc2c1cc1n(c2=O)Cc2c-1nc1cc(F)c(NC(=O)CN)cc1c2CNC(=O)OC(C)(C)C]

To a stirring solution of Fmoc-glycine (98 mg) in dimethylformamide (1 mL) was added HATU (115 mg), HOAt (37 mg) then NMM (85 μL). This solution was stirred for 10 min, then Compound 4.31 (28 mg) was added. The reaction was stirred at room temperature for 16 h then quenched with a solution of 20% piperidine in dimethylformamide (1 mL) and stirred for an additional 20 min. Purification was accomplished as described in General Procedure 9, using a 12 g C18 flash column and eluting with a 5 to 40% CH₃CN/H₂O+0.1% TFA gradient to provide the title compound as a white solid (TFA salt, 25 mg, 67% yield).

LC/MS: Calc'd m/z=567.2 for C₂₈H₃₀FN₆₀₇, found [M+H]⁺=568.4.

¹H NMR (300 MHz, MeOD) δ 9.01 (d, J=8.3 Hz, 1H), 7.83 (d, J=11.9 Hz, 1H), 7.52 (s, 1H), 5.57 (d, J=16.4 Hz, 1H), 5.38 (d, J=16.3 Hz, 1H), 5.27 (d, J=3.1 Hz, 2H), 4.80 (s, 2H), 4.10 (s, 2H), 1.97 (q, J=7.4 Hz, 2H), 1.50 (s, 9H), 1.02 (t, J=7.3 Hz, 3H). 4.33: tert-butyl (((S)-9-(2-((S)-2-(2-(2-aminoacetamido)acetamido)-3-phenylpropanamido)acetamido)-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)carbamate (Compound 4.33)

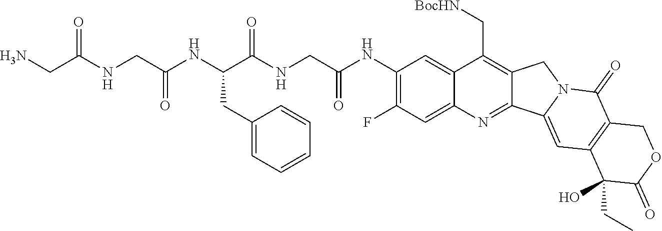 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CN)cc1c2CNC(=O)OC(C)(C)C]

To a stirring solution of Fmoc-GGF-OH (28 mg) and HATU (20 mg) in a mixture of DMF (0.2 mL) and dichloromethane (1.8 mL) was added Compound 4.32 (25 mg) followed by DIPEA (32 μL). This solution was stirred for 15 min at room temperature, quenched with a solution of 20% piperidine in dimethylformamide (0.250 mL), stirred for an additional 20 min, then partially concentrated in vacuo. Purification was accomplished as described in General Procedure 9, using a 12 g C18 flash column and eluting with a 10 to 45% CH₃CN/H₂O+0.1% TFA gradient to provide the title compound as a white solid (TFA salt, 22 mg, 64% yield).

LC/MS: Calc'd m/z=828.3 for C₄₁H₄₅FN₈O₁₀, found [M+H]⁺=829.6.

### 4.34: (S)—N-(2-(((S)-11-(aminomethyl)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-2-oxoethyl)-2-(1-(2,5-dioxo-2,5-dihydro-1H-pyrrol-1-yl)-12,15-dioxo-3,6,9-trioxa-13,16-diazaoctadecan-18-amido)-3-phenylpropanamide (MT-GGFG-Compound 145)

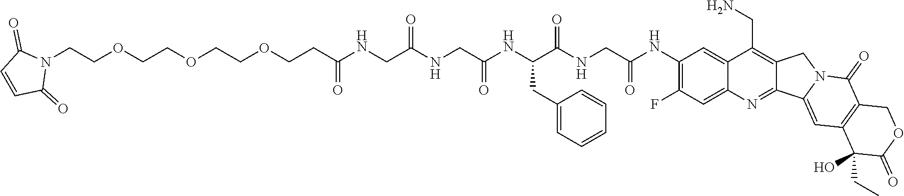 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN]

The title compound was prepared according to Procedure 6 followed by Procedure 8 starting from Compound 4.33 (15 mg). Preparative HPLC purification of the intermediate Boc-protected compound was accomplished as described in General Procedure 9, eluting with a 10 to 50% CH₃CN/H₂O+0.1% TFA gradient. The title compound was obtained post Boc-deprotection as a white-solid (TFA salt, 8.5 mg, 52% yield).

LC/MS: Calc'd m/z=1011.4 for C₄₉H₅₃FN₈O₁₅, found [M+H]⁺=1012.6.

¹H NMR (300 MHz, MeOD) δ 9.04 (d, J=8.0 Hz, 1H), 8.40 (d, J=5.7 Hz, 1H), 8.21 (d, J=7.7 Hz, 1H), 8.05 (d, J=11.5 Hz, 1H), 7.67 (s, 1H), 7.42-7.03 (m, 5H), 6.81 (s, 2H), 5.63 (d, J=16.4 Hz, 1H), 5.51 (s, 1H), 5.43 (d, J=16.5 Hz, 1H), 4.81 (s, 2H), 4.75-4.58 (m, 1H), 4.29-4.10 (m, 2H), 3.98-3.81 (m, 4H), 3.78-3.71 (m, 2H), 3.71-3.63 (m, 2H), 3.62-3.53 (m, 9H), 3.14-2.98 (m, 1H), 2.54 (t, J=6.0 Hz, 2H), 2.08-1.93 (m, 2H), 1.03 (t, J=7.3 Hz, 3H). 4.355: (9H-fluoren-9-yl)methyl(S)-(2-((4-ethyl-8-fluoro-4-hydroxy-3,14-dioxo-11-(piperidin-1-ylmethyl)-3,4,12,14-tetrahydro-1H-pyrano[3′,4′:6,7]indolizino[1,2-b]quinolin-9-yl)amino)-2-oxoethyl)carbamate (Compound 4.35)

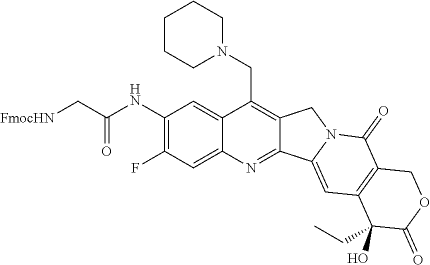

To a solution of Fmoc-Gly-OH (100.9 mg, 0.34 mmol) in dimethylformamide (550 μL) was added NMM (0.112 mL,1.02 mmol) and HATU (0.103 g, 0.272 mmol). This solution was stirred at room temperature for 20 min, then a solution of Compound 148 (32.5 mg, 0.068 mmol) in DMF (250 μL) was added, and the reaction mixture was stirred for 16 h. Purification was accomplished as described in General Procedure 9, using a 12 g C18 flash column and eluting with a 5 to 40% CH₃CN/H₂O+0.1% TFA gradient. The obtained residue was re-purified according to General Procedure 9, using a 10 g flash column and eluting with a 0 to 10% MeOH/DCM gradient to provide the title compound as a yellow powder (15.3 mg, 30% yield).

LC/MS: Calc'd m/z=757.3 for C₄₃H₄₀FN₅O₇, found [M+H]⁺=758.6.

### 4.36: (S)-2-(1-(2,5-dioxo-2,5-dihydro-1H-pyrrol-1-yl)-12,15-dioxo-3,6,9-trioxa-13,16-diazaoctadecan-18-amido)-N-(2-(((S)-4-ethyl-8-fluoro-4-hydroxy-3,14-dioxo-11-(piperidin-1-ylmethyl)-3,4,12,14-tetrahydro-1H-pyrano[3′,4′:6,7]indolizino[1,2-b]quinolin-9-yl)amino)-2-oxoethyl)-3-phenylpropanamide (MT-GGFG-Compound 148)

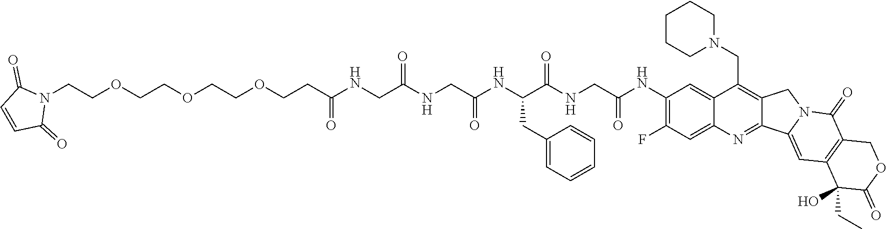 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN1CCCCC1]

To a 50 mL flask containing Compound 4.35 (15.3 mg, 0.02 mmol) was added a solution of 20% piperidine in DMF (2.0 mL). This solution was stirred at room temperature for 5 min then evaporated to dryness. The obtained residue was then dissolved in 10% DMF/DCM (1.0 mL), then NMM (5.50 μL, 0.05 mmol), Compound 4.4 (11.2 mg, 0.02 mmol) and HATU (8.7 mg, 0.02 mmol) were added. This solution was stirred for 45 min, then partially evaporated. Preparative HPLC purification was accomplished as described in General Procedure 9, eluting with a 15 to 45% CH₃CN/H₂O+0.1% TFA gradient to give the title product as a yellow powder (7.8 mg, 33% yield).

LC/MS: Calc'd m/z=1079.4 for C₅₄H₆₂FN₉O₁₄, found [M+H]⁺=1080.8.

¹H NMR (300 MHz, MeOD) δ 8.99 (d, J=8.2 Hz, 1H), 7.52 (d, J=12.3 Hz, 1H), 7.39-7.25 (m, 5H), 7.25-7.17 (m, 1H), 6.79 (s, 2H), 5.53 (d, J=16.4 Hz, 1H), 5.33 (d, J=16.5 Hz, 1H), 4.80-4.72 (m, 1H), 4.32-4.11 (m, 2H), 3.98-3.79 (m, 6H), 3.76 (t, J=6.0 Hz, 2H), 3.66-3.60 (m, 2H), 3.62-3.49 (m, 10H), 3.15-3.03 (m, 1H), 2.65-2.47 (m, 6H), 1.96 (q, J=7.4 Hz, 2H), 1.72-1.57 (m, 4H), 1.57-1.42 (m, 2H), 1.03 (t, J=7.4 Hz, 3H). 4.37. tert-butyl (2-((2-(((S)-1-((2-((4-(N—(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)sulfamoyl)phenyl)amino)-2-oxoethyl)amino)-1-oxo-3-phenylpropan-2-yl)amino)-2-oxoethyl)amino)-2-oxoethyl)carbamate (Compound 4.37)

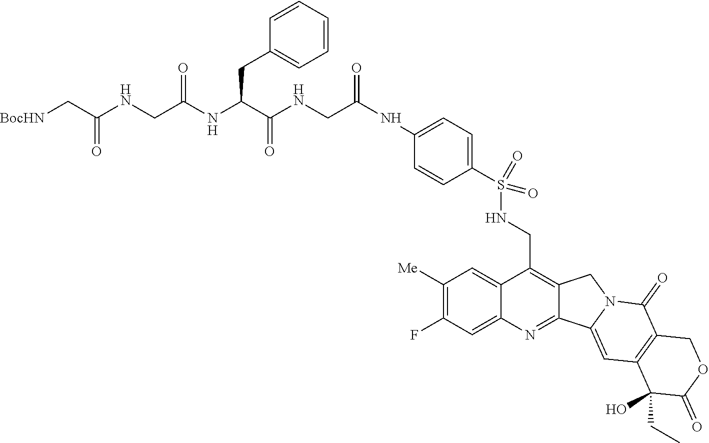 [SMILES: CC[C@@]1(O)C(=O)OCc2c1cc1n(c2=O)Cc2c-1nc1cc(F)c(C)cc1c2CNS(=O)(=O)c1ccc(NC(=O)CNC(=O)[C@H](Cc2ccccc2)NC(=O)CNC(=O)CNC(=O)OC(C)(C)C)cc1]

The title compound was prepared according to General Procedure 7 starting from Compound 127 (46 mg). Preparative HPLC purification was accomplished as described in General Procedure 9, eluting with a 10 to 60% CH₃CN/H₂O+0.1% TFA gradient to provide the title compound as a white solid (7.2 mg, 21% yield).

LC/MS: Calc'd m/z=983.0 for C₄₈H₅₁N₈₀₁₂S, found [M+H]⁺=983.9.

### 4.38: (S)-2-(1-(2,5-dioxo-2,5-dihydro-1H-pyrrol-1-yl)-12,15-dioxo-3,6,9-trioxa-13,16-diazaoctadecan-18-amido)-N-(2-((4-(N—(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)sulfamoyl) phenyl)amino)-2-oxoethyl)-3-phenylpropanamide (MT-GGFG-Compound 127)

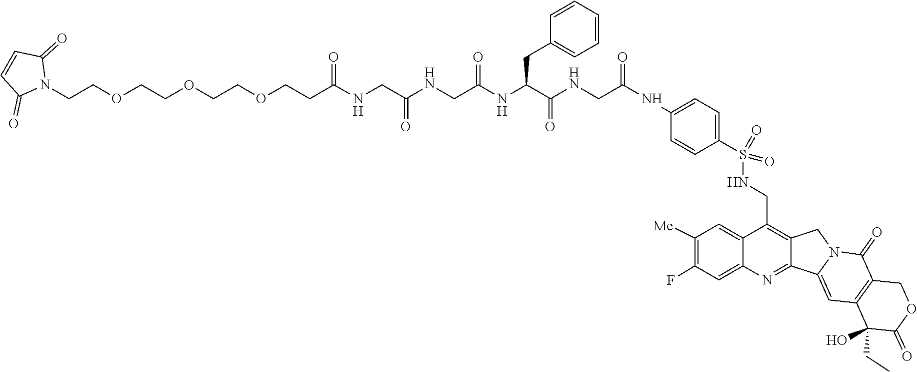 [SMILES: CC[C@@]1(O)C(=O)OCc2c1cc1n(c2=O)Cc2c-1nc1cc(F)c(C)cc1c2CNS(=O)(=O)c1ccc(NC(=O)CNC(=O)[C@H](Cc2ccccc2)NC(=O)CNC(=O)CNC(=O)CCOCCOCCOCCN2C(=O)C=CC2=O)cc1]

The title compound was prepared according to Procedure 6 followed by Procedure 8 starting from Compound 4.37 (7.2 mg). Preparative HPLC purification was accomplished as described in General Procedure 9, eluting with a 10 to 50% CH₃CN/H₂O+0.1% TFA gradient to provide the title compound as a white solid (1 mg, 12% yield).

LC/MS: Calc'd m/z=1166.2 for C₅₆H₆₀FN₉O₁₆, found [M+H]⁺=1167.1

### 4.39: (9H-fluoren-9-yl)methyl ((7S)-1-((3-azabicyclo[3.1.1]heptan-6-yl)oxy)-7-benzyl-3,6,9,12-tetraoxo-2,5,8,11-tetraazatridecan-13-yl)carbamate (Compound 4.39)

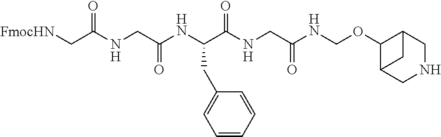

To a stirring solution of Compound 4.6 (44 mg) in dichloromethane (2 mL) was added 3-azabicyclo[3.1.1]heptan-6-ol (5.3 mg) followed by trifluoracetic acid (0.4 mL). After 30 min the reaction was concentrated in vacuo. Purification was accomplished as described in General Procedure 9, using a 10 g flash column and eluting with a 0 to 20% dichloromethane/methanol gradient to provide the title compound as a white solid (14.7 mg, 46% yield).

LC/MS: Calc'd m/z=682.8 for C₃₇H₄₂N₆O₇, found [M+H]⁺=683.6.

### 4.40: (2S)-2-(2-(2-aminoacetamido)acetamido)-N-(2-((((3-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-3-azabicyclo[3.1.1]heptan-6-yl)oxy)methyl)amino)-2-oxoethyl)-3-phenylpropanamide (Compound 4.40)

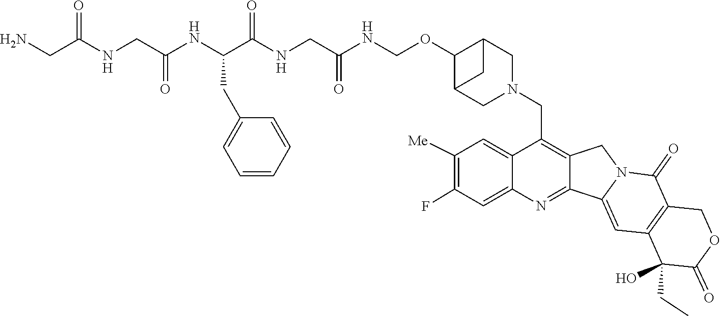 [SMILES: CC[C@@]1(O)C(=O)OCc2c1cc1n(c2=O)Cc2c-1nc1cc(F)c(C)cc1c2CN1CC2CC(C1)C2OCNC(=O)CNC(=O)[C@H](Cc1ccccc1)NC(=O)CNC(=O)CN]

The title compound was prepared according to General Procedure 1 starting from Compound 1.1 (3 mg, 0.007 mmol) and Compound 4.39 (14.7 mg, 0.022 mmol) and utilizing 200 μL DMF. Following complete consumption of Compound 1.1, a solution of 20% piperidine in DMF (200 μL) was added and this solution was stirred at room temperature for 10 min.

Preparative HPLC purification was accomplished as described in General Procedure 9, eluting with a 20 to 37% CH₃CN/H₂O+0.1% TFA gradient to give the title compound as an off-white solid (TFA salt, 1.8 mg, 29% yield).

LC/MS: Calc'd m/z=852.9 for C₄₄H₄₉FN₈₀₉, found [M+H]⁺=853.7.

### 4.41: (2S)-2-(1-(2,5-dioxo-2,5-dihydro-1H-pyrrol-1-yl)-12,15-dioxo-3,6,9-trioxa-13,16-diazaoctadecan-18-amido)-N-(2-((((3-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-3-azabicyclo[3.1.1]heptan-6-yl)oxy)methyl)amino)-2-oxoethyl)-3-phenylpropanamide (MT-GGFG-AM-Compound 117)

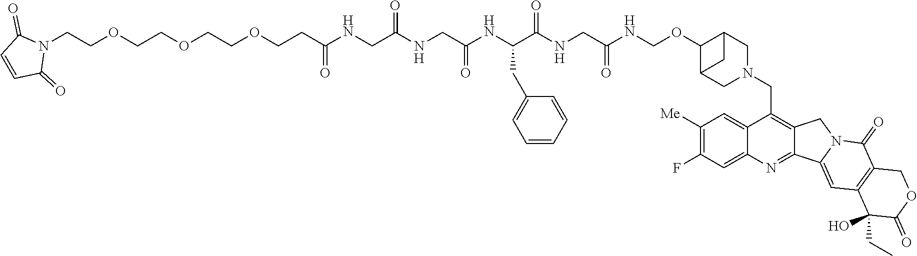 [SMILES: CC[C@@]1(O)C(=O)OCc2c1cc1n(c2=O)Cc2c-1nc1cc(F)c(C)cc1c2CN1CC2CC(C1)C2OCNC(=O)CNC(=O)[C@H](Cc1ccccc1)NC(=O)CNC(=O)CNC(=O)CCOCCOCCOCCN1C(=O)C=CC1=O]

The title compound was prepared according to Procedure 8 starting from Compound 4.40 (1.8 mg). Preparative HPLC purification was accomplished as described in General Procedure 9, eluting with a 25 to 45% CH₃CN/H₂O+0.1% TFA gradient to provide the title compound as a white-solid (TFA salt, 0.5 mg, 22% yield).

LC/MS: Calc'd m/z=1135.5 for C₅₇H₆₆FN₉O₁₅, found [M+H]⁺=1136.3.

### 4.42: (9H-fluoren-9-yl)methyl (S)-(9-benzyl-1-(3-fluoroazetidin-3-yl)-5,8,11,14-tetraoxo-2-oxa-4,7,10,13-tetraazapentadecan-15-yl)carbamate (Compound 4.42)

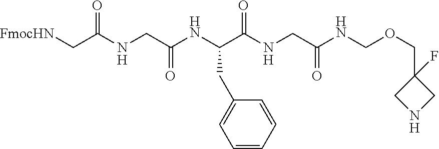

To a stirring solution of Compound 4.6 (144 mg) in dichloromethane (2 mL) was added (3-fluoroazetidin-3-yl)methanol (16 mg) followed by trifluoracetic acid (0.4 mL). After 30 min the reaction was concentrated in vacuo. Purification was accomplished as described in General Procedure 9, using a 10 g flash column and eluting with a 0 to 20% dichloromethane/methanol gradient to provide the title compound as a white solid (55 mg, 54% yield).

LC/MS: Calc'd m/z=674.7 for C₃₅H₃₉N₆F₀₇, found [M+H]⁺=675.6.

### 4.43: (S)-2-(2-(2-aminoacetamido)acetamido)-N-(2-((((1-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-3-fluoroazetidin-3-yl)methoxy)methyl)amino)-2-oxoethyl)-3-phenylpropanamide (Compound 4.43)

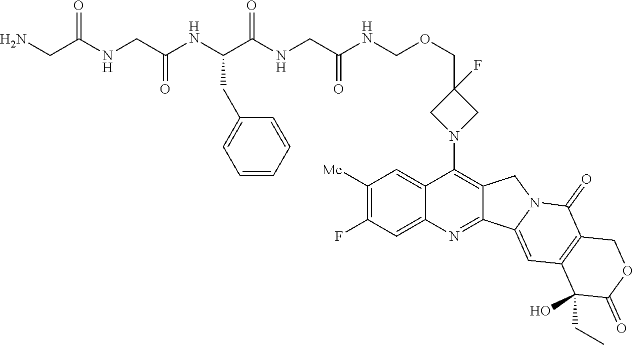 [SMILES: CC[C@@]1(O)C(=O)OCc2c1cc1n(c2=O)Cc2c-1nc1cc(F)c(C)cc1c2N1CC(F)(COCNC(=O)CNC(=O)[C@H](Cc2ccccc2)NC(=O)CNC(=O)CN)C1]

The title compound was prepared according to General Procedure 1 starting from Compound 1.1 (11.6 mg, 0.027 mmol) and Compound 4.42 (55 mg, 0.082 mmol) and utilizing 500 μL DMF. Following complete consumption of Compound 1.1, a solution of 20% piperidine in DMF (500 μL) was added and this solution was stirred at room temperature for 10 min.

Preparative HPLC purification was accomplished as described in General Procedure 9, eluting with a 25 to 32% CH₃CN/H₂O+0.1% TFA gradient to give the title compound as an off-white solid (TFA salt, 8.1 mg, 28% yield).

LC/MS: Calc'd m/z=844.3 for C₄₂H₄₆F₂N₈₀₉, found [M+H]⁺=845.3 4.44: (S)-2-(1-(2,5-dioxo-2,5-dihydro-1H-pyrrol-1-yl)-12,15-dioxo-3,6,9-trioxa-13,16-diazaoctadecan-18-amido)-N-(2-((((1-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-3-fluoroazetidin-3-yl)methoxy)methyl)amino)-2-oxoethyl)-3-phenylpropanamide (MT-GGFG-AM-Compound 118)

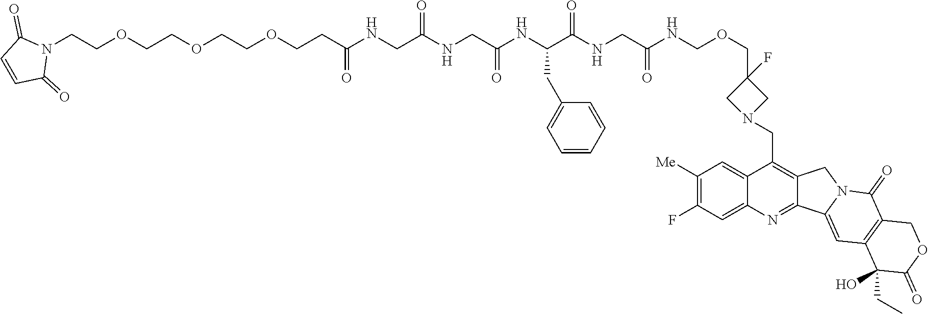 [SMILES: CC[C@@]1(O)C(=O)OCc2c1cc1n(c2=O)Cc2c-1nc1cc(F)c(C)cc1c2CN1CC(F)(COCNC(=O)CNC(=O)[C@H](Cc2ccccc2)NC(=O)CNC(=O)CNC(=O)CCOCCOCCOCCN2C(=O)C=CC2=O)C1]

The title compound was prepared according to Procedure 8 starting from Compound 4.43 (8.1 mg). Preparative HPLC purification was accomplished as described in General Procedure 9, eluting with a 25 to 45% CH₃CN/H₂O+0.1% TFA gradient to provide the title compound as a white-solid (TFA salt, 2.9 mg, 28% yield).

LC/MS: Calc'd m/z=1127.4 for C₅₅H₆₃F₂N₉₀₁₅, found [M+H]⁺=1128.8.

### 4.45: (S)-10-BENZYL-23-(2,5-DIOXO-2,5-DIHYDRO-JH-PYRROL-1-YL)-6,9,12,15,18-PENTAOXO-3-OXA-5,8,11,14,17-PENTAAZATRICOSYL (((S)-4-ETHYL-8-FLUORO-4-HYDROXY-9-METHYL-3,14-DIOXO-3,4,12,14-TETRAHYDRO-1H-PYRANO[3′,4′:6,7]INDOLIZINO[1,2-B]QuINOLIN-11-YL)METHYL)CARBAMATE (MC-GGFG-AM-COMPOUND 139)

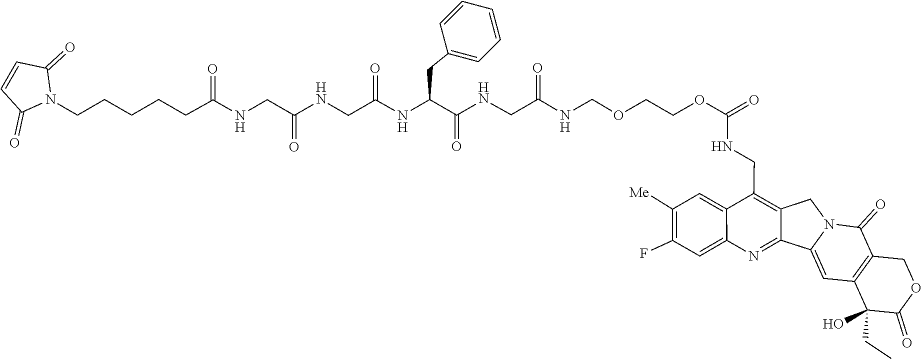 [SMILES: CC[C@@]1(O)C(=O)OCc2c1cc1n(c2=O)Cc2c-1nc1cc(F)c(C)cc1c2CNC(=O)OCCOCNC(=O)CNC(=O)[C@H](Cc1ccccc1)NC(=O)CNC(=O)CNC(=O)CCCCCN1C(=O)C=CC1=O]

To Compound 4.27 (450 mg) was added a solution of 2,5-dioxopyrrolidin-1-yl 6-(2,5-dioxopyrrol-1-yl)hexanoate (130 mg) and N-ethyldiisopropylamine (250 μL) in DMF (10 mL). This solution was stirred at room temperature for 30 min then concentrated to −1 mL volume. Purification was accomplished as described in General Procedure 9 first using a 60 g C18 flash column and eluting with a 10 to 60% CH₃CN/H₂O+0.1% TFA gradient followed by preparative HPLC of impure fractions using a 20 to 50% CH₃CN/H₂O+0.1% TFA gradient to provide the title compound as a white solid (320 mg, 66% yield).

LC/MS: Calc'd m/z=1037.4 for C₅₁H₅₆FN₉O₁₄, found [M+H]⁺=1038.6.

¹H NMR (300 MHz, MeOD) δ 8.10 (d, J=8.1 Hz, 2H), 8.01 (s, 1H), 7.95 (d, J=7.0 Hz, 1H), 7.74 (d, J=10.4 Hz, 1H), 7.66 (s, 1H), 7.56 (s, 1H), 7.32-7.10 (m, 5H), 6.69 (s, 2H), 5.63 (d, J=16.4 Hz, 1H), 5.46 (s, 2H), 5.32 (s, 1H), 5.28 (d, J=16.5 Hz, 1H), 4.88 (s, 2H), 4.67 (d, J=6.4 Hz, 2H), 4.48 (d, J=7.1 Hz, 2H), 4.15 (t, J=4.2 Hz, 2H), 3.92 (dd, J=17.1, 6.2 Hz, 2H), 3.83-3.57 (m, 6H), 3.46 (t, J=7.1 Hz, 2H), 3.16 (dd, J=14.0, 5.9 Hz, 1H), 2.95 (dd, J=13.9, 8.9 Hz, 1H), 2.53 (s, 3H), 2.21 (t, J=7.6 Hz, 2H), 1.97-1.79 (m, 2H), 1.58 (dp, J=15.0, 7.6 Hz, 4H), 1.29 (dd, J=16.6, 9.3 Hz, 3H), 1.01 (t, J=7.3 Hz, 3H). 4.46: tert-butyl (S)-(2-((4-ethyl-8-fluoro-4-hydroxy-3,14-dioxo-3,4,12,14-tetrahydro-1H-pyrano[3′,4′:6,7]indolizino[1,2-b]quinolin-9-yl)amino)-2-oxoethyl)carbamate (Compound 4.46)

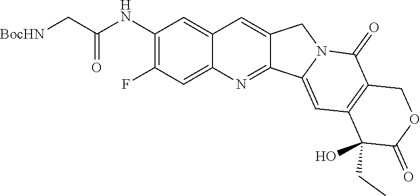 [SMILES: CC[C@@]1(O)C(=O)OCc2c1cc1n(c2=O)Cc2cc3cc(NC(=O)CNC(=O)OC(C)(C)C)c(F)cc3nc2-1]

A solution of Compound 140 (860 mg, 1.7 mmol, TFA salt), Boc-Gly-OH (760 mg, 4.3 mmol), HATU (1.6 g, 4.1 mmol), and N-ethyldiisopropylamine (0.6 mL) in DMF (4 mL) was stirred at room temperature for 24 h then poured into water (50 mL). The resulting solid was collected by filtration, redissolved in 10% MeOH/DCM and purification was accomplished as described in General Procedure 9, using a 30 g silica column and eluting with a 0 to 10% MeOH/DCM to provide the title compound as a yellow solid (750 mg, 80% yield).

LC/MS: Calc'd m/z=538.5 for C₂₇H₂₇FN₄O₇, found [M+H]⁺=539.4.

¹H NMR (300 MHz, MeOD) δ 8.84 (d, J=8.4 Hz, 1H), 8.52 (s, 1H), 8.00 (s, 1H), 7.87 (d, J=12.1 Hz, 1H), 7.62 (s, 1H), 5.60 (d, J=16.3 Hz, 1H), 5.40 (d, J=16.4 Hz, 1H), 5.27 (s, 2H), 4.02 (s, 2H), 1.99 (dt, J=8.7, 6.7 Hz, 2H), 1.52 (s, 9H), 1.03 (t, J=7.4 Hz, 3H). 4.47: (S)-1-((2-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-2-oxoethyl)amino)-1-oxo-3-phenylpropan-2-aminium (Compound 4.47)

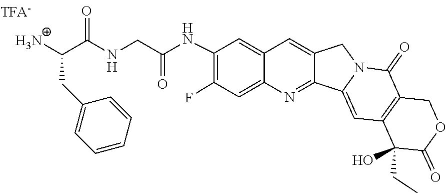 [SMILES: CC[C@@]1(O)C(=O)OCc2c1cc1n(c2=O)Cc2cc3cc(NC(=O)CNC(=O)[C@@H]([NH3+])Cc4ccccc4)c(F)cc3nc2-1]

The title compound was prepared in three steps from Compound 4.46 (750 mg). The Boc protecting group was cleaved in neat TFA (2 mL) followed by precipitation in Et₂O (50 mL). The solid was collected by filtration and added to a solution of 2,5-dioxopyrrolidin-1-yl (2S)-2-[(tert-butoxycarbonyl)amino]-3-phenylpropanoate (340 mg, 1.1 equiv) and N-ethyldiisopropylamine (300 μL) in DMF (1.7 mL). This solution was stirred at room temperature for 30 min then pipetted into Et₂O (50 mL). The precipitate was collected by filtration, dried under vacuum then dissolved in neat TFA (2 mL). After 20 min, Et₂O (50 mL) was added and the precipitate collected by filtration to provide the title compound as a yellow solid (531 mg, 54% yield).

LC/MS: Calc'd m/z=585.2 for C₃₁H₂₈FN₅O₆, found [M+H]⁺=586.1.

### 4.48: 2-((2-(((S)-1-((2-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-2-oxoethyl)amino)-1-oxo-3-phenylpropan-2-yl)amino)-2-oxoethyl)amino)-2-oxoethan-1-aminium (Compound 4.48)

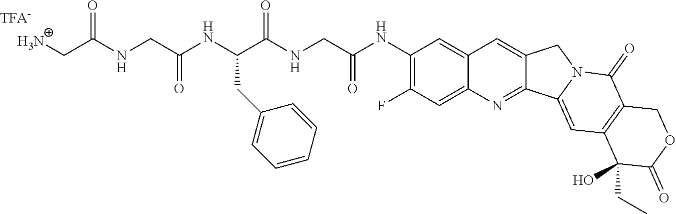 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)C[NH3+])c(F)cc3nc2-1]

To Compound 4.47 (490 mg) was added a solution of Boc-gly-gly-NHS (250 mg, 1.1 equiv) and N-ethyldiisopropylamine (250 μL) in DMF (3 mL). This solution was stirred at room temperature for 30 min then pipetted into Et₂O (50 mL). The precipitate was collected by filtration then dissolved in neat TFA (2 mL). After 20 min, Et₂O (50 mL) was added and the precipitate collected by filtration to provide the title compound as a yellow solid (500 mg, 88% yield).

LC/MS: Calc'd m/z=699.2 for C₃₅H₃₄FN₇₀₈, found [M+H]⁺=700.4.

### 4.49: 6-(2,5-dioxo-2,5-dihydro-1H-pyrrol-1-yl)-N-(2-((2-(((S)-1-((2-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-2-oxoethyl)amino)-1-oxo-3-phenylpropan-2-yl)amino)-2-oxoethyl)amino)-2-oxoethyl)hexanamide (MC-GGFG-Compound 140)

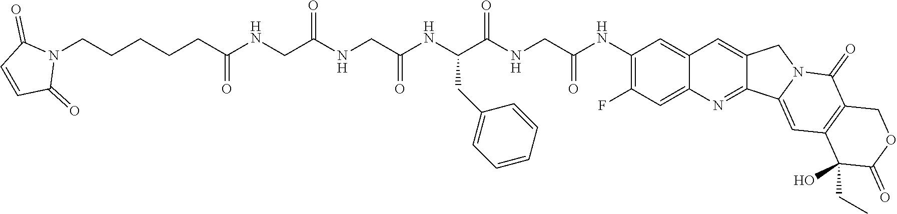 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)CCCCCN4C(=O)C=CC4=O)c(F)cc3nc2-1]

To Compound 4.48 (500 mg) was added a solution of 2,5-dioxocyclopentyl 6-(2,5-dioxopyrrol-1-yl)hexanoate (210 mg, 1.1 equiv) and N-ethyldiisopropylamine (215 μL) in DMF (4 mL). This solution was stirred at room temperature for 30 min then pipetted into Et₂O (50 mL).

The precipitate was collected by filtration then dissolved in DMF (2 mL). Preparative HPLC purification was accomplished as described in General Procedure 9, eluting with a 24 to 38% CH₃CN/H₂O+0.1% TFA gradient to provide the title compound as a yellow solid (190 mg, 40% yield).

LC/MS: Calc'd m/z=892.9 for C₄₅H₄₅FN₈O₁₁, found [M+H]⁺=893.6.

¹H NMR (300 MHz, CD₃CN) δ 8.67 (d, J=8.4 Hz, 1H), 8.44 (s, 1H), 7.78 (d, J=12.1 Hz, 1H), 7.41 (s, 1H), 7.30 (d, J=4.3 Hz, 4H), 7.26-7.16 (m, 1H), 6.72 (s, 2H), 5.52 (d, J=16.4 Hz, 1H), 5.31 (d, J=16.4 Hz, 1H), 5.12 (s, 2H), 4.64 (dd, J=9.7, 5.0 Hz, 1H), 4.11 (d, J=3.2 Hz, 2H), 3.87-3.68 (m, 4H), 3.37 (t, J=7.1 Hz, 2H), 3.00 (dd, J=14.0, 9.7 Hz, 1H), 2.20 (t, J=7.6 Hz, 2H), 1.49 (dq, J=19.5, 7.4 Hz, 4H), 1.22 (p, J=7.6, 7.1 Hz, 2H), 0.94 (t, J=7.3 Hz, 3H). 4.50: tert-butyl (S)-(2-((4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamate (Compound 4.50)

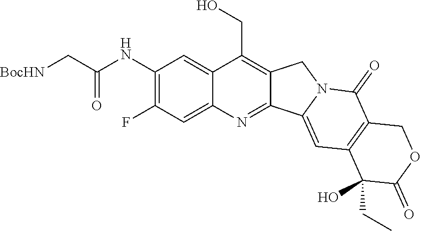 [SMILES: CC[C@@]1(O)C(=O)OCc2c1cc1n(c2=O)Cc2c-1nc1cc(F)c(NC(=O)CNC(=O)OC(C)(C)C)cc1c2CO]

A solution of Compound 4.46 (1.8 g), iron (II) sulfate heptahydrate (1.4 g, 1.5 equiv), and sulfuric acid (450 μL, 2.5 equiv) in MeOH (33 mL) was heated to 60° C. and hydrogen peroxide (1.25 mL, 12 equiv) was added dropwise over 10 min. This solution was heated for another 20 min then cooled to room temperature and poured into ice water (˜200 mL). The brown precipitate was collected by filtration and the filtrate was quenched with saturated aqueous Na₂S₂O₃. MeOH was evaporated and the solution allowed to stand for 2 h while a second brown precipitate formed.

This precipitate was collected by filtration and the combined precipitates were purified as described in General Procedure 9 using a 50 g silica column and eluting with a 0 to 15% MeOH/DCM gradient to provide the title compound as a yellow solid (860 mg, 45% yield).

LC/MS: Calc'd m/z=568.5 for C₂₈H₂₉FN₄O₈, found [M+H]⁺=569.7.

### 4.51: (S)-1-((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)amino)-1-oxo-3-phenylpropan-2-aminium (Compound 4.51)

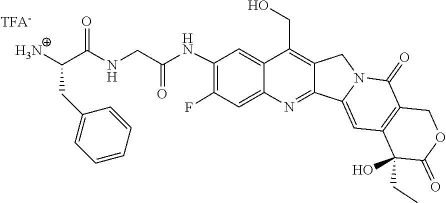 [SMILES: CC[C@@]1(O)C(=O)OCc2c1cc1n(c2=O)Cc2c-1nc1cc(F)c(NC(=O)CNC(=O)[C@@H]([NH3+])Cc3ccccc3)cc1c2CO]

The title compound was prepared in three steps from Compound 4.50 (750 mg). The Boc protecting group was cleaved in neat TFA (2 mL) followed by precipitation in Et₂O (100 mL). The solid was collected by filtration and added to a solution of 2,5-dioxopyrrolidin-1-yl (2S)-2-[(tert-butoxycarbonyl)amino]-3-phenylpropanoate (600 mg, 1.1 equiv) and N-ethyldiisopropylamine (300 μL) in DMF (7 mL). This solution was stirred at room temperature for 30 min then pipetted into Et₂O (100 mL). The precipitate was collected by filtration, dried under vacuum then dissolved in neat TFA (2 mL). After 20 min, Et₂O (100 mL) was added and the precipitate collected by filtration to provide the title compound as a yellow solid (756 mg, 78% yield).

LC/MS: Calc'd m/z=615.2 for C₃₂H₃₀FN₅O₇, found [M+H]⁺=616.3.

### 4.52: 2-((2-(((S)-1-((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)amino)-1-oxo-3-phenylpropan-2-yl)amino)-2-oxoethyl)amino)-2-oxoethan-1-aminium (Compound 4.52)

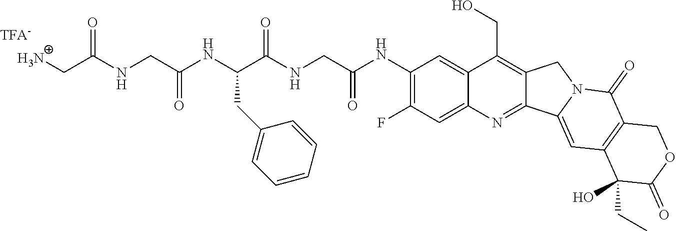 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)C[NH3+])cc1c2CO]

To Compound 4.51 (756 mg) was added a solution of Boc-gly-gly-NHS (375 mg, 1.1 equiv) and N-ethyldiisopropylamine (400 μL) in DMF (5 mL). This solution was stirred at room temperature for 30 min then pipetted into Et₂O (75 mL). The precipitate was collected by filtration then dissolved in neat TFA (4 mL). After 20 min, Et₂O (100 mL) was added and the precipitate collected by filtration to provide the title compound as a yellow solid (826 mg, 95% yield).

LC/MS: Calc'd m/z=729.2 for C₃₆H₃₆FN₇₀₉, found [M+H]⁺=730.2.

### 4.53: 6-(2,5-dioxo-2,5-dihydro-1H-pyrrol-1-yl)-N-(2-((2-(((S)-1-((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)amino)-1-oxo-3-phenylpropan-2-yl)amino)-2-oxoethyl)amino)-2-oxoethyl)hexanamide (MC-GGFG-Compound 141)

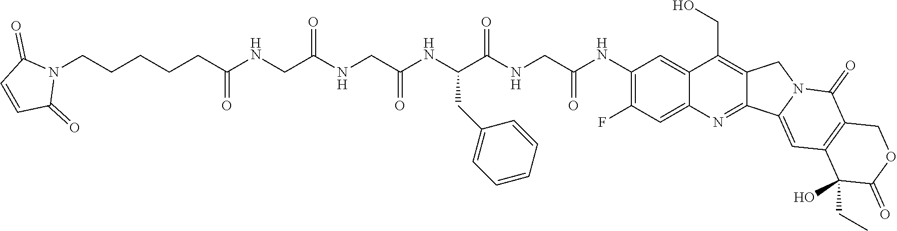 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCCCCN3C(=O)C=CC3=O)cc1c2CO]

To Compound 4.52 (826 mg) was added a solution of 2,5-dioxocyclopentyl 6-(2,5-dioxopyrrol-1-yl)hexanoate (382 mg, 1.1 equiv) and N-ethyldiisopropylamine (300 μL) in DMF (5.5 mL). This solution was stirred at room temperature for 30 min then pipetted into Et₂O (100 mL). The precipitate was collected by filtration then dissolved in DMF (2 mL). Preparative HPLC purification was accomplished as described in General Procedure 9, eluting with a 25 to 40% CH₃CN/H₂O+0.1% TFA gradient to provide the title compound as a yellow solid (370 mg, 35% yield).

LC/MS: Calc'd m/z=922.9 for C₄₆H₄₇FN₈O₁₂, found [M+H]⁺=923.8.

¹H NMR (300 MHz, CD₃CN) δ 8.63 (d, J=8.4 Hz, 1H), 7.67 (d, J=11.9 Hz, 1H), 7.38-7.27 (m, 5H), 7.24 (d, J=4.3 Hz, 1H), 6.72 (s, 2H), 5.48 (d, J=16.4 Hz, 1H), 5.28 (d, J=16.3 Hz, 1H), 5.24-5.01 (m, 4H), 4.65 (dd, J=9.7, 4.9 Hz, 1H), 4.13 (s, 2H), 3.85-3.75 (m, 3H), 3.37 (t, J=7.1 Hz, 2H), 3.00 (dd, J=14.0, 9.8 Hz, 1H), 2.21 (t, J=7.6 Hz, 2H), 1.51 (dp, J=22.0, 7.4 Hz, 4H), 1.22 (p, J=7.4, 7.0 Hz, 2H), 0.94 (t, J=7.3 Hz, 3H). 4.54: tert-butyl ((S)-1-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-1-oxopropan-2-yl)carbamate (Compound 4.54)

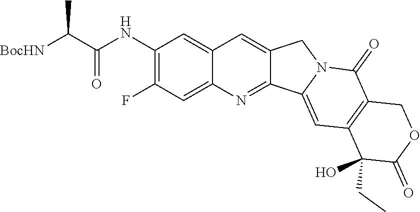 [SMILES: CC[C@@]1(O)C(=O)OCc2c1cc1n(c2=O)Cc2cc3cc(NC(=O)[C@H](C)NC(=O)OC(C)(C)C)c(F)cc3nc2-1]

To Compound 3.4 (500 mg, 1.0 mmol) was added TFA (4 mL) and this solution was allowed to stand at rt for 1 h, then Et₂O (100 mL) was added, and the precipitate was collected by filtration. This solid was taken up in DMF (3.4 mL) and Boc-Ala-OH (590 mg, 3.1 mmol, 3 equiv) and HATU (1.2 g, 3.1 mmol, 3equiv) were added followed by N-ethyldiisopropylamine (0.9 mL, 5.2 mmol, 5 equiv). This solution was stirred at rt for 3 days then poured into ice water (50 mL) and the precipitate was collected by filtration to give the title compound as a brown solid (125 mg, 22% yield).

LC/MS: Calc'd m/z=552.6 for C₂₈H₂₉FN₄O₇, found [M+H]⁺=553.7.

### 4.55: (S)-2-amino-N—((S)-1-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-1-oxopropan-2-yl)-3-methylbutanamide (Compound 4.55)

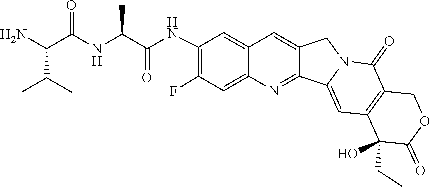 [SMILES: CC[C@@]1(O)C(=O)OCc2c1cc1n(c2=O)Cc2cc3cc(NC(=O)[C@H](C)NC(=O)[C@@H](N)C(C)C)c(F)cc3nc2-1]

To Compound 4.54 (125 mg, 0.225 mmol) in a 100 mL round bottom flask was added TFA (2 mL). This solution was allowed to stand for 10 min, then Et₂O (50 mL) was added, and the precipitate collected by filtration. The resulting orange solid was added to a solution of Boc-Val-NHS (78 mg, 0.25 mmol, 1.1 equiv) and N-ethyldiisopropylamine (80 μL, 0.45 mmol, 2 equiv) in DMF (2 mL). This solution was stirred at rt for 30 min, then pipetted into Et₂O (40 mL) in a 50 mL falcon tube and the precipitate was collected by centrifugation and decanting of the Et₂₀. The pellet was dissolved in TFA (2 mL) and allowed to stand for 10 min prior to the addition of Et₂O (40 mL). The precipitate was collected by centrifugation and decanting the Et₂₀. The pellet was dried under high vacuum to give the title compound as an orange solid (135 mg, 90% yield over 3 steps).

LC/MS: Calc'd m/z=551.2 for C₂₈H₃₀FN₅₀₆, found [M+H]⁺=552.2.

### 4.56: 6-(2,5-dioxo-2,5-dihydro-1H-pyrrol-1-yl)-N—((S)-1-(((S)-1-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-1-oxopropan-2-yl)amino)-3-methyl-1-oxobutan-2-yl)hexanamide (MC-VA-Compound 140)

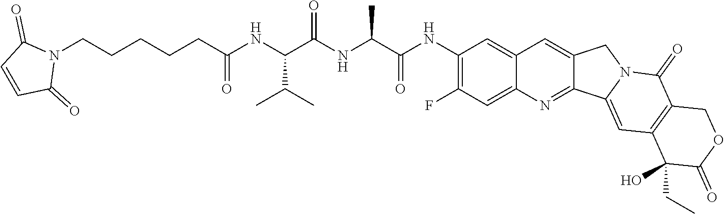 [SMILES: CC[C@@]1(O)C(=O)OCc2c1cc1n(c2=O)Cc2cc3cc(NC(=O)[C@H](C)NC(=O)[C@@H](NC(=O)CCCCCN4C(=O)C=CC4=O)C(C)C)c(F)cc3nc2-1]

To Compound 4.55 (20 mg, 0.03 mmol) was added a solution of 2,5-dioxopyrrolidin-1-yl 6-(2,5-dioxopyrrol-1-yl)hexanoate (11 mg, 0.036 mmol) and N-ethyldiisopropylamine (10 μL) in DMF (1 mL). This solution was stirred at rt for 30 min then purified directly. Preparative HPLC purification was accomplished as described in General Procedure 9, eluting with a 20 to 60% CH₃CN/H₂O+0.1% TFA gradient to provide the title compound as a yellow solid (8.8 mg, 40% yield).

LC/MS: Calc'd m/z=744.8 for C₃₈H₄₁FN₆₀₉, found [M+H]⁺=745.6.

¹H NMR (300 MHz, 10% D₂O/CD₃CN) δ 8.60 (d, J=8.5 Hz, 1H), 8.31 (s, 1H), 7.96 (d, J=6.5 Hz, 1H), 7.65 (d, J=12.0 Hz, 1H), 7.37-7.26 (m, 2H), 6.75 (s, 2H), 5.45 (d, J=16.6 Hz, 1H), 5.25 (d, J=16.3 Hz, 1H), 5.04 (d, J=4.0 Hz, 2H), 4.78-4.58 (m, 1H), 4.30-4.13 (m, 1H), 2.32-2.16 (m, 2H), 2.10 (dt, J=13.6, 6.8 Hz, 1H), 1.88 (q, J=7.4 Hz, 2H), 1.57 (dq, J=15.5, 7.6 Hz, 4H), 1.45 (d, J=7.1 Hz, 3H), 1.26 (tt, J=10.1, 6.1 Hz, 2H), 1.05-0.83 (m, 9H). 4.57. 2,5-dioxopyrrolidin-1-yl 6-(((S)-1-(((S)-1-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-1-oxopropan-2-yl)amino)-3-methyl-1-oxobutan-2-yl)amino)-6-oxohexanoate (NHC—C-VA-Compound 140)

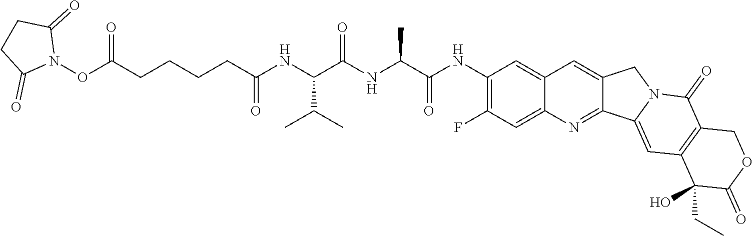 [SMILES: CC[C@@]1(O)C(=O)OCc2c1cc1n(c2=O)Cc2cc3cc(NC(=O)[C@H](C)NC(=O)[C@@H](NC(=O)CCCCC(=O)ON4C(=O)CCC4=O)C(C)C)c(F)cc3nc2-1]

To Compound 4.55 (20 mg, 0.03 mmol) was added a solution of bis(2,5-dioxopyrrolidin-1-yl) adipate (30 mg, 0.09 mmol, 3 equiv) and N-ethyldiisopropylamine (10 μL) in DMF (1 mL).

This solution was stirred at rt for 30 min then purified directly. Preparative HPLC purification was accomplished as described in General Procedure 9, eluting with a 25 to 35% CH₃CN/H₂O+0.1% TFA gradient to provide the title compound as a yellow solid (4.1 mg, 18% yield).

LC/MS: Calc'd m/z=776.8 for C₃₈H₄₁FN₆₀₁₁, found [M+H]⁺=777.6.

¹H NMR (300 MHz, 10% D₂O/CD₃CN) δ 8.65 (dd, J=8.4, 2.3 Hz, 1H), 8.38 (s, 1H), 7.95 (d, J=6.5 Hz, 1H), 7.72 (d, J=12.0 Hz, 1H), 7.37 (d, J=11.8 Hz, 2H), 5.58-5.19 (m, 2H), 5.10 (s, 2H), 4.78-4.56 (m, 1H), 4.23 (dd, J=8.4, 7.0 Hz, 1H), 2.80 (s, 4H), 2.65 (t, J=6.9 Hz, 2H), 2.39-2.22 (m, 2H), 2.11 (q, J=6.8 Hz, 1H), 1.94-1.81 (m, 2H), 1.79-1.57 (m, 4H), 1.45 (d, J=7.1 Hz, 3H), 1.10-0.78 (m, 9H). 4.58: (S)-2-(32-azido-5-oxo-3,9,12,15,18,21,24,27,30-nonaoxa-6-azadotriacontanamido)-N—((S)-1-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-1-oxopropan-2-yl)-3-methylbutanamide (2-((Azido-PEG8-carbamoyl)methoxy)acetamido-VA-Compound 140)

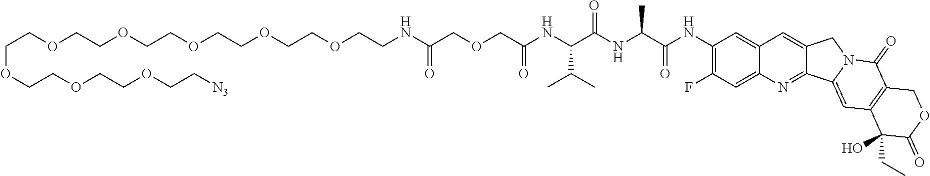 [SMILES: CC[C@@]1(O)C(=O)OCc2c1cc1n(c2=O)Cc2cc3cc(NC(=O)[C@H](C)NC(=O)[C@@H](NC(=O)COCC(=O)NCCOCCOCCOCCOCCOCCOCCOCCOCCN=[N+]=[N-])C(C)C)c(F)cc3nc2-1]

A solution of Compound 4.55 (20 mg, 0.03 mmol), 32-azido-5-oxo-3,9,12,15,18,21,24,27,30-nonaoxa-6-azadotriacontanoic acid (17 mg, 0.03 mmol), and HATU (13 mg, 0.03 mmol) in DMF (300 μL) was cooled to 0° C. and N-ethyldiisopropylamine (16 μL, 0.09 mmol) was added. This solution was stirred for 30 min the purified directly. Preparative HPLC purification was accomplished as described in General Procedure 9, eluting with a 20 to 50% CH₃CN/H₂O+0.1% TFA gradient to provide the title compound as a yellow solid (13.7 mg, 42% yield).

LC/MS: Calc'd m/z=1088.2 for C₅₀H₇₀FN₉O₁₇, found [M+H]⁺=1088.8.

¹H NMR (300 MHz, 10% D₂O/CD₃CN) δ 8.62 (d, J=8.5 Hz, 1H), 8.33 (s, 1H), 7.66 (d, J=12.2 Hz, 1H), 7.35 (s, 1H), 5.52-5.18 (m, 2H), 5.04 (s, 2H), 4.72 (q, J=7.1 Hz, 1H), 4.32 (d, J=7.3 Hz, 1H), 4.15-3.98 (m, 4H), 3.68-3.48 (m, 35H), 3.41-3.34 (m, 6H), 2.18 (h, J=6.8 Hz, 1H), 1.88 (q, J=7.4 Hz, 2H), 1.47 (d, J=7.1 Hz, 3H), 1.13-0.84 (m, 9H).

### 4.58: tert-butyl (2-(pyridin-2-yldisulfaneyl)ethyl)carbamate (Compound 4.58)

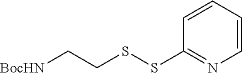 [SMILES: CC(C)(C)OC(=O)NCCSSc1ccccn1]

The title compound was prepared as described in Wang, et al., Nano Lett., 2014, 14(10):5577-5583.

### 4.59: tert-butyl (2-((2-hydroxyethyl)disulfaneyl)ethyl)carbamate (Compound 4.59)

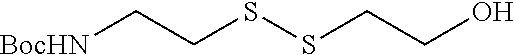 [SMILES: CC(C)(C)OC(=O)NCCSSCCO]

To a solution of Compound 4.58 (200 mg, 0.7 mmol) in DCM (1.4 mL) was added β-mercaptoethanol (50 μL, 0.7 mmol) and this solution was stirred at rt for 5 h. The solution was diluted with DCM (10 mL), washed with a water (3×10 mL), dried over Na₂SO₄, and concentrated to an oil. Purification was accomplished as described in General Procedure 9, using a 10 g silica column, and eluting with a 0 to 10% MeOH/DCM to give the title compound as a colorless solid (212 mg, 82% yield).

LC/MS: Calc'd m/z=253.1 for C₁₁H₂₃NO₃S₂, found [M+H,−Boc]⁺=154.0.

¹H NMR (300 MHz, Chloroform-d) δ 4.94 (s, 1H), 3.91 (t, J=5.7 Hz, 2H), 3.49 (q, J=6.4 Hz, 2H), 2.86 (dt, J=23.7, 6.1 Hz, 4H), 2.15 (s, 2H), 1.47 (s, 9H).

### 4.60: 2-((2-(3-(2,5-dioxo-2,5-dihydro-1H-pyrrol-1-yl)propanamido)ethyl)disulfaneyl)ethyl (4-nitrophenyl) carbonate (Compound 4.60)

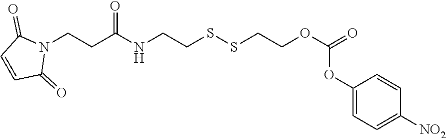 [SMILES: O=C(CCN1C(=O)C=CC1=O)NCCSSCCOC(=O)Oc1ccc([N+](=O)[O-])cc1]

To Compound 4.59 (212 mg, 0.837 mmol) in a 25 mL round bottom flask was added a 4M HCl/dioxane solution (5 mL) and the solution was stirred at rt for 30 min, then evaporated to dryness. The residue was suspended in EtOAc (10 mL) and evaporated to dryness to give the amine as the HCl salt and as a white powder. To this solid was added a solution of 2,5-dioxopyrrolidin-1-yl 3-(2,5-dioxopyrrol-1-yl)propanoate (245 mg, 0.92 mmol, 1.1 equiv.) and N-ethyldiisopropylamine (0.438 mL, 2.51 mmol) in DMF (1.7 mL). This solution was stirred at rt for 20 min then 4-nitrophenyl carbonate (280 mg, 0.92 mmol) was added and the reaction was then left to stir overnight. Purification of the crude reaction mixture was accomplished as described in General Procedure 9, using a 12 g C18 flash column, and eluting with a 10 to 100% CH₃CN/H₂O+0.1% TFA gradient to provide the title compound as a white solid (141 mg, 36% yield).

LC/MS: Calc'd m/z=469.5 for C₁₈H₁₉N₃O₈S₂, found [M+H]⁺=470.2.

¹H NMR (300 MHz, Chloroform-d) δ 8.37-8.25 (m, 2H), 7.46-7.35 (m, 2H), 6.71 (d, J=2.1 Hz, 2H), 6.32 (s, 1H), 4.55 (t, J=6.6 Hz, 2H), 3.83 (t, J=7.0 Hz, 2H), 3.65-3.50 (m, 2H), 3.09-2.99 (m, 2H), 2.84 (q, J=6.1 Hz, 2H), 2.52 (td, J=7.1, 3.1 Hz, 2H).

### 4.61: 2-((2-(3-(2,5-dioxo-2,5-dihydro-1H-pyrrol-1-yl)propanamido)ethyl)disulfaneyl)ethyl (S)-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)carbamate (DiS-Compound 145)

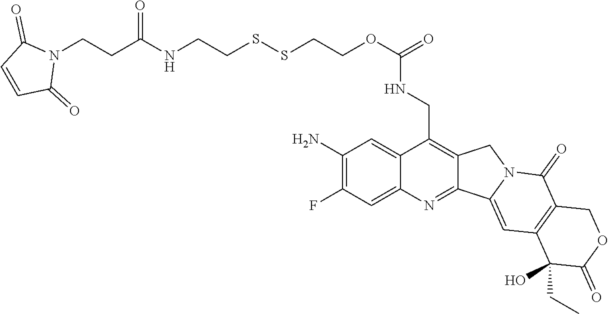 [SMILES: CC[C@@]1(O)C(=O)OCc2c1cc1n(c2=O)Cc2c-1nc1cc(F)c(N)cc1c2CNC(=O)OCCSSCCNC(=O)CCN1C(=O)C=CC1=O]

A solution of Compound 4.60 (18 mg, 0.038 mmol) and N-ethyldiisopropylamine (15 μL, 0.087 mmol) in DMF (300 μL) was added to Compound 145 (13 mg, 0.029 mmol) and this solution was stirred at rt for 20 min. The solution was acidified with an aqueous 1M HCl solution (100 μL) and purified directly. Preparative HPLC purification was accomplished as described in General Procedure 9, eluting with a 20 to 45% CH₃CN/H₂O+0.1% TFA gradient to provide the title compound as a yellow solid (6.8 mg, 32% yield).

LC/MS: Calc'd m/z=740.8 for C₃₃H₃₃FN₆₀₉S₂, found [M+H]⁺=741.5.

¹H NMR (300 MHz, 10% D₂O/CD₃CN) δ 7.63 (d, J=12.1 Hz, 1H), 7.39-7.22 (m, 2H), 6.74 (d, J=6.7 Hz, 2H), 5.50 (d, J=16.2 Hz, 1H), 5.26 (d, J=16.2 Hz, 1H), 5.20 (s, 2H) 4.69 (s, 2H), 4.28 (t, J=6.3 Hz, 2H), 3.62 (t, J=7.0 Hz, 2H), 3.31 (t, J=6.6 Hz, 2H), 2.74-2.64 (m, 2H), 2.35 (t, J=7.0 Hz, 2H), 1.90 (dd, J=15.5, 8.1 Hz, 2H), 1.23-1.04 (m, 6H), 0.93 (t, J=7.4 Hz, 3H).

### Example 5: In Vitro Cytotoxicity Of Camptothecin Analogues

Cytotoxicity of the camptothecin analogues was assessed in vitro as follows.

In vitro potency was assessed on multiple cancer cell lines: SK-BR-3 (breast cancer), SKOV-3 (ovarian cancer), Calu-3 (lung cancer), ZR-75-1 (breast cancer) and MDA-MB-468 (breast cancer). Serial dilutions of camptothecin analogues were prepared in RPMI 1640+10% FBS, and 20 μL of each dilution was added to 384-well plates. Cells cultured in log-phase growth were detached by brief incubation in 0.05% Trypsin and resuspended in respective culturing media at 20,000 cells/mL (with the exception of ZR-75 cells, which were resuspended at 10,000 cells/mL). 50 μL of cell suspension was then added to the plates containing test articles. Cells were incubated with test articles for 4 d at 37° C. (with the exception of ZR-75 cells, which were incubated for 5 d). Growth inhibition was assessed by CellTiter-Glo® (Promega Corporation, Madison, WI) and luminescence was measured on a plate reader. IC50 values were determined by GraphPad Prism (GraphPad Software, San Diego CA).

The results are shown in Table 5.1.

TABLE 5.1
In vitro Cytotoxicity of Camptothecin Analogues (pIC50)

| | pIC50 | | | | |
|---|---|---|---|---|---|
| Camptothecin | | | | | MDA-MB- |
| Analogue | Calu-3 | SKBR-3 | SKOV-3 | ZR-75 | 468 |
| Compound 100 | 8.95 | 8.96 | 8.64 | 8.91 | 8.60 |
| Compound 102 | ND* | 9.03 | 8.67 | 9.10 | 8.63 |
| Compound 104 | 8.28 | 8.57 | 8.11 | 8.59 | 8.58 |
| Compound 122 | 8.43 | 8.95 | 8.04 | 8.89 | 8.96 |
| Compound 132 | 7.29 | 7.64 | 6.98 | 7.81 | 7.57 |
| Compound 106 | 8.34 | 8.47 | 8.13 | 8.42 | 8.03 |
| Compound 108 | 7.39 | 7.53 | 7.26 | 7.73 | 7.32 |
| Compound 101 | ND | 8.97 | 8.80 | 8.97 | 8.59 |
| Compound 103 | 8.63 | 8.77 | 8.36 | 8.62 | 8.33 |
| Compound 105 | 8.35 | 8.44 | 7.97 | 8.51 | 8.56 |
| Compound 107 | 8.39 | 8.51 | ND | 8.56 | 8.41 |
| Compound 109 | ND | 8.24 | 8.15 | 8.30 | 8.09 |
| Compound 134 | 7.61 | 8.02 | 7.11 | 7.92 | 7.94 |
| Compound 138 | 9.41 | 9.32 | 9.21 | 9.71 | 9.06 |
| Compound 124 | 8.77 | 8.92 | ND | 8.64 | 8.46 |
| Compound 136 | 7.58 | 8.40 | 7.29 | 8.30 | 8.20 |
| Compound 133 | 7.92 | 8.17 | 7.13 | 8.01 | 7.97 |
| Compound 135 | 7.60 | 7.91 | ND | 7.88 | 7.97 |
| Compound 137 | 7.20 | 7.77 | ND | 7.73 | 7.55 |
| Compound 123 | 8.43 | 8.72 | 7.71 | 8.61 | 8.70 |
| Compound 125 | 8.55 | 8.88 | 8.15 | 8.88 | 8.42 |
| Compound 128 | ND | 8.28 | 7.74 | 8.37 | 8.08 |
| Compound 126 | 8.53 | 8.96 | 8.20 | 9.14 | 8.71 |
| Compound 130 | 8.78 | 7.83 | 7.62 | ND | 8.19 |
| Compound 129 | 8.28 | 8.51 | 7.69 | 8.58 | 8.40 |
| Compound 127 | 8.03 | 8.48 | 7.84 | 8.28 | 8.42 |
| Compound 110 | 9.09 | 8.88 | 9.03 | 9.07 | 8.81 |
| Compound 111 | 7.71 | 8.07 | 7.78 | 8.04 | 7.54 |
| Compound 112 | 7.76 | 8.18 | 7.70 | 7.92 | 7.87 |
| Compound 113 | 8.52 | 8.71 | 8.37 | 8.66 | 8.47 |
| Compound 139 | 8.15 | 8.92 | 8.30 | 8.97 | 8.82 |
| Compound 140 | 9.51 | 9.50 | 9.34 | 9.48 | 9.15 |
| Compound 141 | 8.99 | 9.46 | 8.55 | 9.51 | 8.84 |
| Compound 142 | 8.89 | 9.20 | 8.89 | 9.12 | 8.89 |
| Compound 143 | 9.15 | 9.41 | 8.55 | 9.07 | 9.15 |
| Compound 144 | 7.65 | 9.10 | 7.16 | 7.88 | 7.65 |
| Compound 145 | 9.57 | 9.45 | 9.03 | 9.42 | 9.57 |
| Compound 146 | 8.36 | 8.76 | 7.95 | 8.22 | 8.36 |
| Compound 147 | 7.67 | 8.29 | 7.36 | ND | 7.67 |
| Compound 148 | 9.69 | 9.49 | ND | 9.66 | 9.69 |
| Compound 131 | 8.04 | 8.98 | ND | 9.02 | 8.04 |
| Compound 149 | 8.20 | 8.50 | 8.00 | 8.74 | 8.20 |
| Compound 150 | ND | 8.10 | 7.20 | 8.19 | ND |
| Compound 151 | 7.94 | 8.57 | 7.34 | 8.23 | 7.94 |
| Compound 152 | 8.83 | 8.49 | 8.54 | ND | 8.30 |
| Compound 153 | 9.94 | 9.29 | 9.03 | ND | ND |
| Compound 114 | 10.03 | 9.97 | 9.75 | ND | 9.38 |
| Compound 115 | 8.89 | 8.59 | 8.42 | ND | 8.38 |
| Compound 117 | 9.79 | 10.02 | 9.77 | ND | 9.33 |
| Compound 118 | 9.03 | 8.82 | 8.84 | ND | 8.73 |
| Compound 119 | 8.93 | 8.38 | 8.43 | ND | 8.56 |
| Compound 120 | 10.00 | 8.96 | 9.60 | ND | 9.72 |
| Compound 121 | 9.84 | 9.83 | 9.71 | ND | 9.57 |
| Compound 116 | 9.10 | 8.15 | 7.90 | ND | 8.03 |

*ND = not determined

### Example 6: Production of M3-H1L1 and M3-H18L6 ANTI-GPC3 Antibodies

The VH and VL polypeptide sequences of M3-H₁L1 and M3-H18L6 antibodies were identified from International Patent Publication No. WO 2021/226321. Constructs were produced in full-size antibody (FSA) format containing two identical full-length heavy chains and two identical kappa light chains. Reference antibodies codrituzumab and BMS-986182 were also constructed.

### 6.1 Cloning

The full-length heavy chains contained the human CH1-hinge-CH2-CH3 domain sequence of GHG1*01 (SEQ ID NO:24; see Table 6.1) or human CH-hinge-CH2-CH3 domain sequence of IGHG1*03 (SEQ ID NO:25; see Table 6.1) and the light chains contained the human kappa CL sequence of IGKC*01 (SEQ ID NO:26; see Table 6.1).

TABLE 6
Table 6.1: Constant Heavy and Light Chain Sequences

| | | SEQ ID |
|---|---|---|
| | Sequence | NO: |
| CH1-hinge- | ASTKGPSVFPLAPSSKSTSGGTAALGCLVKDYFPEPVTVSWNSGALTS | 24 |
| CH2-CH3 | GVHTFPAVLQSSGLYSLSSVVTVPSSSLGTQTYICNVNHKPSNTKVDK | |
| (IGHG1*01) | KVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPSRD | |
| | ELTKNQVSLTCLVKGFYPSDIAVEWESNGQPENNYKTTPPVLDSDGSF | |
| | FLYSKLTVDKSRWQQGNVFSCSVMHEALHNHYTQKSLSLSPG | |
| CH1-hinge- | ASTKGPSVFPLAPSSKSTSGGTAALGCLVKDYFPEPVTVSWNSGALTS | 25 |
| CH2-CH3 | GVHTFPAVLQSSGLYSLSSVVTVPSSSLGTQTYICNVNHKPSNTKVDKR | |
| (IGHG1*03) | VEPKSCDKTHTCPPCPAPELLGGPSVFLFPPKPKDTLMISRTPEVTCVV | |
| | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSREE | |
| | MTKNQVSLTCLVKGFYPSDIAVEWESNGQPENNYKTTPPVLDSDGSFF | |
| | LYSKLTVDKSRWQQGNVFSCSVMHEALHNHYTQKSLSLSPG | |
| CL_(kappa) | RTVAAPSVFIFPPSDEQLKSGTASVVCLLNNFYPREAKVQWKVDNALQ | 26 |
| (IGKC*01) | SGNSQESVTEQDSKDSTYSLSSTLTLSKADYEKHKVYACEVTHQGLSS | |
| | PVTKSFNRGEC | |

Table 6.2: VH and VL Sequences of M3-H1L1, M3-H18L6 and Reference Antibodies

| | | SEQ ID |
|---|---|---|
| | Sequence | NO |
| M3-HIL1 VH | EVQLVQSGAEVKKPGSSVKVSCKASGYTFTDYEMHWVRQAPGQG | 27 |
| | LEWIGAIDPETGDTAYNQKFKGRATLTADKSTSTAYMELSSLRSEDT | |
| | AVYYCTRYFSFAYWGQGTLVTVSS | |
| M3-HIL1 VL | DVQMTQSPSSVSASVGDRVTITCRSSQSLRHSNGNTYLQWYQQKPG | 28 |
| | KAPKLLIYKVSNRFSGVPSRFSGSGSGTDFTLTISSLQPEDFATYFCY | |
| | QSKHVPYTFGQGTKVEIK | |
| M3-H18L6 | QVQLVQSGAEVKKPGSSVKVSCKASGYTFTDYEMHWVKQAPGHG | 29 |
| VH | LEWIGAIDPETGDTAYNQKFKGKATLTADTSTSTAYMELSSLRSEDT | |
| | AVYYCTRYFSFAYWGQGTLVTVSS | |
| M3-H18L6 VL | EIVLTQSPATLSLSPGERATISCRSSQSLRHSNGNTYLQWYQQKPGQ | 30 |
| | APRLLIYKVSNRFSGVPARFSGSGSGTDFTLTISSLEPEDFAVYFCYQ | |
| | SKHVPYTFGGGTKVEIK | |
| Codrituzumab | QVQLVQSGAEVKKPGASVKVSCKASGYTFTDYEMHWVRQAPGQG | 31 |
| VH | LEWMGALDPKTGDTAYSQKFKGRVTLTADKSTSTAYMELSSLTSE | |
| | DTAVYYCTRFYSYTYWGQGTLVTVSS | |
| Codrituzumab | DVVMTQSPLSLPVTPGEPASISCRSSQSLVHSNRNTYLHWYLQKPGQ | 32 |
| VL | SPQLLIYKVSNRFSGVPDRFSGSGSGTDFTLKISRVEAEDVGVYYCS | |
| | QNTHVPPTFGQGTKLEIK | |
| BMS-986182 | EVQLVQSGAEVKKPGESLKISCKGSGYSFTSYWIAWVRQMPGKGLE | 33 |
| VH | WMGIIFPGDSDTRYSPSFQGQVTISADRSIRTAYLQWSSLKASDTAL | |
| | YYCARTREGYFDYWGQGTLVTVSS | |
| BMS-986182 | EIVLTQSPGTLSLSPGERATLSCRAVQSVSSSYLAWYQQKPGQAPRL | 34 |
| VL | LIYGASSRATGIPDRFSGSGSGTDFTLTISRLEPEDFAVYYCQQYGSS | |
| | PTFGGGTKVEIK | |

Table 6.3: HC and LC Sequences of M3-HIL1, M3-H18L6 and Reference Antibodies

| | | SEQ ID |
|---|---|---|
| | Sequence | NO |
| M3-H1L1 HC | EVQLVQSGAEVKKPGSSVKVSCKASGYTFTDYEMHWVRQAPGQG | 35 |
| | LEWIGAIDPETGDTAYNQKFKGRATLTADKSTSTAYMELSSLRSEDT | |
| | AVYYCTRYFSFAYWGQGTLVTVSSASTKGPSVFPLAPSSKSTSGGT | |
| | AALGCLVKDYFPEPVTVSWNSGALTSGVHTFPAVLQSSGLYSLSSV | |
| | VTVPSSSLGTQTYICNVNHKPSNTKVDKKVEPKSCDKTHTCPPCPAP | |
| | ELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKV | |
| | SNKALPAPIEKTISKAKGQPREPQVYTLPPSRDELTKNQVSLTCLVK | |
| | GFYPSDIAVEWESNGQPENNYKTTPPVLDSDGSFFLYSKLTVDKSR | |
| | WQQGNVFSCSVMHEALHNHYTQKSLSLSPG | |
| M3-HIL1 LC | DVQMTQSPSSVSASVGDRVTITCRSSQSLRHSNGNTYLQWYQQKPG | 36 |
| | KAPKLLIYKVSNRFSGVPSRFSGSGSGTDFTLTISSLQPEDFATYFCY | |
| | QSKHVPYTFGQGTKVEIKRTVAAPSVFIFPPSDEQLKSGTASVVCLL | |
| | NNFYPREAKVQWKVDNALQSGNSQESVTEQDSKDSTYSLSSTLTLS | |
| | KADYEKHKVYACEVTHQGLSSPVTKSFNRGEC | |
| M3-H18L6 | QVQLVQSGAEVKKPGSSVKVSCKASGYTFTDYEMHWVKQAPGHG | 37 |
| HC | LEWIGAIDPETGDTAYNQKFKGKATLTADTSTSTAYMELSSLRSEDT | |
| | AVYYCTRYFSFAYWGQGTLVTVSSASTKGPSVFPLAPSSKSTSGGT | |
| | AALGCLVKDYFPEPVTVSWNSGALTSGVHTFPAVLQSSGLYSLSSV | |
| | VTVPSSSLGTQTYICNVNHKPSNTKVDKKVEPKSCDKTHTCPPCPAP | |
| | ELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKV | |
| | SNKALPAPIEKTISKAKGQPREPQVYTLPPSRDELTKNQVSLTCLVK | |
| | GFYPSDIAVEWESNGQPENNYKTTPPVLDSDGSFFLYSKLTVDKSR | |
| | WQQGNVFSCSVMHEALHNHYTQKSLSLSPG | |
| M3-H18L6 LC | EIVLTQSPATLSLSPGERATISCRSSQSLRHSNGNTYLQWYQQKPGQ | 38 |
| | APRLLIYKVSNRFSGVPARFSGSGSGTDFTLTISSLEPEDFAVYFCYQ | |
| | SKHVPYTFGGGTKVEIKRTVAAPSVFIFPPSDEQLKSGTASVVCLLN | |
| | NFYPREAKVQWKVDNALQSGNSQESVTEQDSKDSTYSLSSTLTLSK | |
| | ADYEKHKVYACEVTHQGLSSPVTKSFNRGEC | |
| Codrituzumab | QVQLVQSGAEVKKPGASVKVSCKASGYTFTDYEMHWVRQAPGQG | 39 |
| HC | LEWMGALDPKTGDTAYSQKFKGRVTLTADKSTSTAYMELSSLTSE | |
| | DTAVYYCTRFYSYTYWGQGTLVTVSSASTKGPSVFPLAPSSKSTSG | |
| | GTAALGCLVKDYFPEPVTVSWNSGALTSGVHTFPAVLQSSGLYSLS | |
| | SVVTVPSSSLGTQTYICNVNHKPSNTKVDKKVEPKSCDKTHTCPPCP | |
| | APELLGGPSVFLFPPKPKDTLMISRTPEVTCVVVDVSHEDPEVKFNW | |
| | YVDGVEVHNAKTKPREEQYNSTYRVVSVLTVLHQDWLNGKEYKC | |
| | KVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKS | |
| | RWQQGNVFSCSVMHEALHNHYTQKSLSLSPG | |
| Codrituzumab | DVVMTQSPLSLPVTPGEPASISCRSSQSLVHSNRNTYLHWYLQKPGQ | 40 |
| LC | SPQLLIYKVSNRFSGVPDRFSGSGSGTDFTLKISRVEAEDVGVYYCS | |
| | QNTHVPPTFGQGTKLEIKRTVAAPSVFIFPPSDEQLKSGTASVVCLLN | |
| | NFYPREAKVQWKVDNALQSGNSQESVTEQDSKDSTYSLSSTLTLSK | |
| | ADYEKHKVYACEVTHQGLSSPVTKSFNRGEC | |
| BMS-986182 | EVQLVQSGAEVKKPGESLKISCKGSGYSFTSYWIAWVRQMPGKGLE | 41 |
| HC | WMGIIFPGDSDTRYSPSFQGQVTISADRSIRTAYLQWSSLKASDTAL | |
| | YYCARTREGYFDYWGQGTLVTVSSASTKGPSVFPLAPSSKSTSGGT | |
| | AALGCLVKDYFPEPVTVSWNSGALTSGVHTFPAVLQSSGLYSLSSV | |
| | VTVPSSSLGTQTYICNVNHKPSNTKVDKRVEPKSCDKTHTCPPCPAP | |
| | ELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKV | |
| | SNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSR | |
| | WQQGNVFSCSVMHEALHNHYTQKSLSLSPG | |
| BMS-986182 | EIVLTQSPGTLSLSPGERATLSCRAVQSVSSSYLAWYQQKPGQAPRL | 42 |
| LC | LIYGASSRATGIPDRFSGSGSGTDFTLTISRLEPEDFAVYYCQQYGSS | |
| | PTFGGGTKVEIKRTVAAPSVFIFPPSDEQLKSGTASVVCLLNNFYPRE | |
| | AKVQWKVDNALQSGNSQESVTEQDSKDSTYSLSSTLTLSKADYEK | |
| | HKVYACEVTHQGLSSPVTKSFNRGEC | |

Each VH domain sequence was appended to the human CH1-hinge-CH2-CH3 domain sequence of IGHG1*01, to provide M3-H₁L1 and M3-H18L6 heavy chain sequences, as well as reference antibody codrituzumab full heavy chain sequences. The VH sequence for reference antibody BMS-986182 was appended to the human CH1-hinge-CH₂—CH₃ domain sequence of IGHG1*03, to provide the full heavy chain sequence. Each VL domain sequence was appended to the human kappa CL sequence of IGKC*01 to provide M3-H₁L1 and M3-H₁₈L6, light chain sequences as well as reference antibody codrituzumab and BMS-986182 light chain sequences. All sequences were reverse translated to DNA, codon optimized for mammalian expression and gene synthesized.

Heavy chain vector inserts comprising a signal peptide (artificially designed sequence: MRPTWAWWLFLVLLLALWAPARG (SEQ ID NO:43, Barash et al., 2002, Biochem and Biophys Res. Comm., 294:835-842) and the heavy chain clone terminating at residue G446 (EU numbering) of the CH₃ domain were ligated into a pTT5 vector to produce heavy chain expression vectors. Light chain vector inserts comprising the same signal peptide were ligated into a pTT5 vector to produce light chain expression vectors. The resulting heavy and light chain expression vectors were sequenced to confirm correct reading frame and sequence of the coding DNA.

### 6.2 Expression and Purification of Antibodies

Antibodies were prepared as described in the following two methods. Two lots of M3-H18L6 (v37574) were prepared, each using one of the two methods. No substantive differences were observed when comparing the antibody product resulting from each method.

Method 1: Expression and Purification of M3-H18L6 (v37574) and reference antibodies codrituzumab (v37575) and BMS-986182 (v33624)

Reference antibody BMS-986182 (v33624) was expressed and purified according to Method 1, with minor deviations related to expression volume and with PBS as the final buffer.

The heavy and light chains of v37574 (M3 H₁₈L6) and v37575 (codrituzumab) were expressed in 1 L cultures of CHO-3E7 cells. Briefly, CHO-3E7 cells, at a density of 1.7-2.2×10⁶ cells/mL, viability >95%, were cultured at 37° C. in FreeStyle™ F₁₇ medium (Thermo Fisher Scientific, Waltham, MA) supplemented with 4 mM glutamine (Hyclone™ SH30034.01) and 0.1% Pluronica F-68 (Gibco™/Thermo Fisher Scientific, Waltham, MA). A total volume of 1 L CHO-3E7 cells+1×antibiotic/antimycotics (GE Life Sciences, Marlborough, MA) was transfected with a total of 1 mg DNA (500 μg of antibody DNA and 500 μg of GFP/AKT/stuffer DNA) using PEI-MAX® (Polyscience, Inc., Philadelphia, PA) at a DNA:PEI ratio of 1:4 (w/w). Twenty-four hours after the addition of the DNA-PEI mixture, 0.5 mM valproic acid (final concentration)+1% w/v Tryptone (final concentration) were added to the cells, which were then transferred to 32° C. and incubated for 6 more days prior to harvesting.

Protein-A purification was performed using HiTrap™ MabSelect™ SuRe™ columns (Cytiva, Marlborough, MA). Clarified supernatant samples were loaded on cleaned-in-place (CIP'd) with NaOH and equilibrated in Dulbecco's PBS (DPBS) columns. The columns were washed with DPBS before the elution. Protein was eluted with 100 mM sodium citrate buffer pH 3.0. The eluted fractions were pH adjusted by adding 10% (v/v) 1 M HEPES (pH ˜10.6-10.7) to yield a final pH of 6-7. Antibodies were further purified by preparatory SEC chromatography on a HiLoad™ 26/600 Superdex™ 200 μg column (Cytiva, Marlborough, MA) in H₆NaCl (50 mM Histidine, 150 mM NaCl, pH 6.0) mobile phase following protein-A purification. Samples were buffer exchanged into H₆Su buffer (50 mM Histidine, 9% w/v sucrose, pH6.0). Protein was quantitated based on absorbance at 280 nm (A280 nm).

Following purification, purity of samples was assessed by electrophoresis under non-reducing and reducing conditions using the High Throughput Protein Express assay and Caliper LabChip® GXII or GXII Touch HT (Perkin Elmer, Waltham, MA). Procedures were carried out according to HT Protein Express LabChip® User Guide version 2 with the following modifications. Antibody samples, at either 2 L or 5 L (concentration range 5-2000 ng/μL), were added to separate wells in 96 well plates (BioRad, Hercules, CA) along with 7 L of HT Protein Express Sample Buffer (Perkin Elmer, Cat #760328). Antibody samples were then denatured at 70° C. for 15 mins. The LabChip® instrument was operated using the HT Protein Express LabChip® (Perkin Elmer, Waltham, MA) and the Ab-200 assay setting.

The yield post preparatory SEC purification for v37574 was 70 mg and for v37575 was 62.1 mg per 1 L of culture (post protein-A: 92.8 and 84.2 mg/L respectively) and the yield for reference antibody v33624 was 10.7 mg from 2 L culture (or 5.4 mg/L) post protein-A purification (preparatory SEC was not performed). FIG. 1A shows the Caliper electrophoresis results for these antibodies. As can be seen from FIG. 1A, non-reducing (NR) and reducing (R) Caliper reflected a single species corresponding to full-size antibody and intact heavy and light chains for all antibodies.

Method 2: Expression and Purification of M3-H18L6 (v37574) and M3-H₁L1 (v36180)

ExpiCHO™ cells were cultured at 37° C. in ExpiCHO™ expression medium (Thermo Fisher Scientific™, Waltham, MA) on an orbital shaker rotating at 120 rpm in a humidified atmosphere of 8% CO₂. 100 mL expression volumes and 400 mL expression volume in the case of v37574 were used. Each 1 mL of cells at a density of 6×10⁶ cells/mL was transfected with a total of 0.8 μg DNA. Prior to transfection the DNA was diluted in 76.8 L OptiPRO™ SFM (Thermo Fisher, Waltham, MA), after which 3.2 L of ExpiFectamine™ CHO reagent (Thermo Fisher, Waltham, MA) was directly added to make a total volume of 80 μL. After incubation for 1-5 minutes, the DNA-ExpiFectamine™ CHO Reagent complex was added to the cell culture (80 μL complex per 1 mL of cell culture) then incubated in a 120 rpm shaking incubator at 37° C. and 8% CO₂. Following incubation at 37° C. for 18-22 hours, 6 L of ExpiCHO™ Enhancer and 240 μL of ExpiCHO™ Feed (Thermo Fisher, Waltham, MA) were added per 1 mL of culture. Cells were maintained in culture at 37° C. for a total of 8-10 days, after which each culture was harvested by transferring into appropriately sized falcon tubes and centrifuging at 3500 rpm for 15 minutes. In the case of v37574 (Max Titer protocol), cells were transferred to an orbital shaker rotating at 120 rpm in a humidified atmosphere of 5% CO₂ and a temperature of 32° C. On Day 5 post transfection, 160 L of ExpiCHO™ Feed (Thermo Fisher, Waltham, MA) was added again per 1 mL of culture and the cells were maintained at 5% CO₂ and 32° C. After a total of 12-14 days from Day 0 (transfection day), culture was transferred into appropriately sized falcon tubes and centrifuged at 3500 rpm for 15 minutes. Supernatants were filtered using a 0.2 μm polyethersulfone membrane (Thermo Fisher, Waltham, MA), then analyzed by non-reducing SDS-PAGE and Octet (ForteBio).

Protein purification was performed in either batch mode or with the use of an AKTA™ Pure purification system. In batch mode, supernatants from transient transfections were applied to slurries containing 50% MabSelect SuRe™ resin (Cytiva, Marlborough, MA) and incubated overnight at 2-8° C. on an orbital shaker at 150 rpm. The slurries were transferred into chromatography columns and supernatants were allowed to flow through while resins remained in the column. The resins were then washed with at least 5 Bed Volumes (BV) of resin Equilibration buffer (PBS). To elute the targeted proteins, 2.5 BV of Elution Buffer (100 mM sodium citrate buffer pH 3.5) was added to the columns and collected. Elutions were then neutralized by adding 20% (v/v) 1 M Tris pH 9 to reach a final pH of 6-7. In AKTA™ Pure purification mode, supernatants from transient transfections were loaded onto HiTrap MabSelect SuRE LX columns (Cytiva, Marlborough, MA) that were pre-equilibrated with 5 Column Volume (CV) of PBS. After the proteins were captured, the columns were then washed with 10 CV of PBS. The captured proteins were eluted with 5 CV of Elution Buffer (100 mM sodium citrate buffer pH 3.5) in fractions. Pooled fractions were neutralized with 20% (v/v) if 1 M Tris pH 9. The protein content of each elution was determined by 280 nm absorbance measurement using a Nanodrop™. Samples not undergoing preparative SEC were buffer exchanged into PBS buffer. Where preparative SEC was needed, samples were loaded onto a Superdex 200 HiLoad 16/600 column (Cytiva, Marlborough, MA) on an AKTA™ Pure 25 chromatography system (Cytiva, Marlborough, MA) in PBS with a flow rate of 1 mL/min. Fractions of eluted protein were collected based on A280 nm and their purity were analyzed by non-reducing CE-SDS with LabChip™ GXII Touch (Perkin Elmer, Waltham, MA). Protein containing fractions of high purity were pooled and protein in final pools was quantitated based on A280 nm (Nanodrop™) post SEC.

The purity of protein samples was assessed by non-reducing and reducing LabChip™ CE-SDS. LabChip™ GXII Touch (Perkin Elmer, Waltham, MA). Analysis was carried out according to Protein Express Assay User Guide (PerkinElmer, Waltham, MA), with the following modifications. Samples at a concentration range of 5-2000 ng/μL were added to separate wells in 96 well plates (#MSP9631, BioRad, Hercules, CA) along with 7 μL of HT Protein Express Sample Buffer (#CLS920003, Perkin Elmer) and denatured at 90° C. for 5 mins. The LabChip™ instrument was operated using the LabChip™ HT Protein Express Chip (Perkin Elmer #760528) with HT Protein Express 200 assay setting.

Post Protein-A purification, the yield of v36180 was 785 mg/L of culture, for v37574 the yield was 260 mg/L of culture. All antibodies displayed similar Caliper profiles reflective of expected antibody composition.

### 6.3 Quality Assessment of Antibodies

Species homogeneity of the antibodies was assessed by UPLC-SEC after protein-A purification or after preparatory SEC purification (whichever was the final step).

Samples prepared according to Method 1 were analyzed as follows: UPLC-SEC was performed using a Waters Acquity BEH200 SEC column (2.5 mL, 4.6×150 mm, stainless steel, 1.7 m particles) (Waters LTD, Mississauga, ON) set to 30° C. and mounted on a Waters Acquity UPLC™ H-Class Bio system with a photodiode array (PDA) detector. The mobile phase was 200 mM KPO₄, 200 mM KCl, 0.02% Tween-20, pH 7.0 and the flow rate was 0.4 ml/min. Total run time for each injection was 7 min with a total mobile phase volume of 2.8 mL. Elution was monitored by UV absorbance in the range 210-500 nm, and chromatograms were extracted at 280 nm. Peak integration was performed using Waters Empower® 3 software employing the Apex Track™ and detect shoulders features.

FIG. 1B shows the UPLC-SEC profiles for the v37574 and v37575 (post SEC purification) and for v33624 (post Protein A purification). The UPLC-SEC profiles reflected high species homogeneity.

Samples prepared according to Method 2 were analyzed as follows: UPLC-SEC was performed using an Agilent Technologies AdvanceBio SEC300 Å SEC column (7.8×150 mm, 1.7 m particles) (Agilent Technologies, Santa Clara, California) set to 25° C. and mounted on an Agilent Technologies 1260 infinity II system with a DAD detector. Run times consisted of 7 min and a total volume per injection of 7 mL with a running buffer of either PBS pH7.4 or 200 mM KPO₄, 200 mM KCl, pH 7. Elution was monitored by UV absorbance in the range 190-400 nm, and chromatograms were extracted at 280 nm. Peak integration was performed using OpenLAB™ CDS ChemStation™ software.

UPLC-SEC profiles of v36180 and v37574 after protein-A purification reflected high species homogeneity.

### 6.4 Purity Assessment of Antibodies

The apparent purity of antibodies v37574, v37575, and v33624 was assessed using mass spectrometry after protein A purification or preparatory SEC and non-denaturing deglycosylation.

As the antibody samples contained Fc N-linked glycans only, the samples were treated with N-glycosidase F (PNGase-F) only. The purified samples were de-glycosylated with PNGaseF as follows: 20 μg of antibody was diluted to 1 mg/ml with dd (double-distilled) H₂O then 20 μL of 300 mM Tris-HCl pH 8 (for samples in A5Su or H₆Su buffer) or 100 mM Tris-HCl pH 7 (for samples in PBS), as well as 2U PNGaseF (Sigma), was added and the antibody was incubated overnight at 37° C. (final protein concentration of 0.48 mg/mL). After deglycosylation, the samples were stored at 4° C. prior to LC-MS analysis.

The deglycosylated protein samples were analyzed by intact LC-MS using an Dionex UltiMate 3000 HPLC system (Thermo Fisher, Watham, MA) coupled to an LTQ-Orbitrap™ XL mass spectrometer (ThermoFisher, Waltham, MA) (tuned for optimal detection of larger proteins (>50 kDa)) via an Ion Max electrospray source. The samples were injected onto a 2.1×30 mm Poros R2 reverse phase column (Applied Biosystems Corp., Waltham, MA) and resolved using a 0.1% formic acid aq/acetonitrile (degassed) linear gradient consisting of increasing concentration (20-90%) of acetonitrile. The column was heated to 82.5° C. and solvents were heated pre-column to 80° C. to improve protein peak shape. The cone voltage (source fragmentation setting) was approximately 40 V, the FT resolution setting was 7,500 and the scan range was m/z 400-4,000. The LC-MS system performance was evaluated prior to sample analysis using a deglycosylated IgG standard (Waters IgG standard) as well as a deglycosylated mAb standard mix (25:75 half:full sized antibody). For each LC-MS analysis, the mass spectra acquired across the antibody peak (typically 3.9-4.4 minutes) were summed and the entire multiply charged ion envelope (m/z 1,200-4,000) was deconvoluted into a molecular weight profile using the MaxEnt 1 module of MassLynx™ data analysis software (Waters, Milford, MA) (Peak width at half height=1.0, iterations=10, Minimum intensity ratios Left=60%, Right=60%). The apparent amount of each antibody species in each sample was determined from peak heights in the resulting molecular weight profiles.

The results are shown in Table 6.4. All antibody samples were determined to contain about 100% of the desired species.

TABLE 6.4
Purity of Antibodies Determined by LC/MS

| Antibody | Name | % Desired Species |
|---|---|---|
| v37574* | M3-H18L6 | 100 |
| v37575 | codrituzumab | 100 |
| v33624 | BMS-986182 | \hat{ } effectively 100 |

*Sample prepared according to Method 1 only.

\hat{ }89.82% of full-size Ab signal and 10.18% of half-Ab signal, with half-Ab signal likely being a deconvolution artifact

### Example 7: Binding of Antibodies to Human and Cynomolgus GPC3 by Surface Plasmon Resonance

The ability of antibodies to bind to GPC3 was assessed by surface plasmon resonance (SPR) as described below.

The SPR assay for determination of GPC3 affinity of the antibodies was carried out on a Biacore™ T200 SPR system with PBS-T (PBS+0.05% (v/v) Tween 20) running buffer (with 0.5 M EDTA stock solution added to 3.4 mM final concentration) at a temperature of 25° C. CM5 Series S sensor chip, Biacore™ amine coupling kit (NHS, EDC and 1 M ethanolamine), and 10 mM sodium acetate buffers were purchased from Cytiva Life Sciences (Mississauga, ON, Canada). PBS running buffer with 0.05% Tween20 (PBST) was purchased from Teknova Inc. (Hollister, CA). Antigens: recombinant human and cynomolgus GPC3 was purchased from ACROBiosystems (Newark, DE) and SEC purified on a Superdex 200 10/300 GL column (Cytiva) in PBST running buffer at 0.8 mL/min.

Screening of the antibodies for binding to GPC3 antigen was conducted via anti-Fc capture of antibodies, followed by the injection of five concentrations of GPC3. The anti-Fc surface was prepared on a CM5 Series S sensor chip by standard amine coupling methods as described by the manufacturer (Cytiva Life Sciences, Mississauga, ON, Canada). The immobilization of the anti-Fc was performed using goat anti-human IgG (Cat #109-005-098; Jackson Immuno Research, West Grove, PA) at 25 μg/mL in 10 mM sodium acetate buffer pH 4.5 and the Biacore™ T200 immobilization wizard with an amine coupling method aiming for 4000 RUs. Approximately 500-600 RUs of each antibody (1-3 μg/mL) were captured on the goat anti-human IgG surface by injecting at 10 μL/min for 60s. Using single-cycle kinetics, five concentrations of a two-fold dilution series of human or cynomolgus GPC3 starting at 40 nM (or 20 nM) with a blank buffer control were injected at 40 μL/min for 180s of contact time, followed by 180 s (or 300 s) dissociation phase, resulting in sensorgrams with a buffer blank reference. The anti-Fc surface was regenerated to prepare for the next injection cycle by one pulse of 10 mM glycine/HCl pH 1.5 for 180 s at 40 μL/min. Blank-subtracted sensorgrams were analyzed using Biacore™ T200 Evaluation Software v3.0. The blank-subtracted sensorgrams were then fit to the 1:1 Langmuir binding model.

The results are shown in Table 7.1. M3-H₁L1 and M3-H18L6 showed similar affinity for human GPC3 (Kd of 10.5 and 11.1 nM respectively), while reference antibody codrituzumab had approximately 10-fold higher affinity than M3-H18L6 (Kd=0.91 nM). All antibodies exhibited almost identical affinity to cynomolgus GPC3 when compared to human GPC3.

TABLE 7.1
Antigen binding Assessment of Selected
Antibodies (in PBS) by SPR

| | | Kd (nM) | Kd (nM) | Fold difference |
|---|---|---|---|---|
| Antibody | Name | huGPC3 | cynoGPC3 | (hu/cyno) |
| v36180 | M3-H1L1 | 10.5 | 9.67 | 1.1 |
| v37574 | M3-H18L6 | 11.1 | 11.1 | 1.0 |
| v37575 | codrituzumab | 0.91 | 0.91 | 1.0 |

### Example 8: Thermal Stability of Anti-GPC3 Antibodies

The thermal stability of the v37574 and v37575 antibodies, in three different buffers was assessed by differential scanning calorimetry (DSC) as described below. Aliquots were buffer exchanged post protein-A into A5Su and H₆Su, alongside PBS buffer.

400 mL of purified samples primarily at concentrations of 0.4 mg/mL in PBS or A5Su or H₆Su were used for DSC analysis with a VP-Capillary DSC (Malvern Panalytical Inc., Westborough, MA). At the start of each DSC run, 5 buffer blank injections were performed to stabilize the baseline, and a buffer injection was placed before each sample injection for referencing. Each sample was scanned from 20° C. to 100° C. at a 60° C./hr rate, with low feedback, 8 sec filter, 3 min pre-scan thermostat, and 70 psi nitrogen pressure. The resulting thermograms were referenced and analyzed using Origin 7 software (OriginLab Corporation, Northampton, MA) to determine melting temperature (Tm) as an indicator of thermal stability.

The Fab Tm values were determined for M3-H18L6 and codrituzumab and shown in Table 8.1. Thermal stability of M3-H18L6 (Fab Tm of ˜74.6-76.1° C.) is comparable to that of codrituzumab (Fab Tm of ˜75-75.8° C.) and is independent of the buffer composition.

TABLE 8.1
Thermal Stability of M3-H18L6 and codrituzumab

| Antibody | Name | Fab Tm (° C.) |
|---|---|---|
| v37574 in PBS | M3-H18L6 | 76.1 |
| v37574 in A5Su | M3-H18L6 | 74.6 |
| v37574 in H6Su | M3-H18L6 | 74.9 |
| v37575 in PBS | codrituzumab | 75.8 |
| v37575 in A5Su | codrituzumab | 75.0 |
| v37575 in H6Su | codrituzumab | 75.3 |

### Example 9: Developability Assessment of Anti-Gpc3 Antibodies

The isoelectric point, propensity for self-aggregation, and non-specific binding of anti-GPC3 antibodies v36180 (M3-H₁L1) and v37574 (M3-H₁₈L6), were determined and compared to v37575 (codrituzumab) in order to assess the developability of these antibodies. The isoelectric point was measured by capillary isoelectric focusing (clEF), the propensity for self-aggregation was measured by Affinity-capture self-interaction nanoparticle spectroscopy (AC-SINS) and non-specific binding was measured by NS-ELISA, as described below.

Capillary Isoelectric Focusing (cIEF)

cIEF was carried out using Maurice C. (ProteinSimple©) system, System Suitability Kit and Method Development Kit. System suitability standard, fluorescence calibration standard, cartridge and samples were prepared according to vendor's recommendations. The capillary was automatically calibrated with a fluorescence standard preconditioned with Maurice cIEF System Suitability Kit to ensure the capillary was functioning properly. The antibody samples were diluted to a concentration of 0.5 mg/mL in a final volume of 40 μL in Gibco™ Distilled Water, and mixed Maurice cIEF Method Development Kit Samples. The samples were then vortexed, centrifuged and the supernatant pipetted into individual wells of a 96-well plate. All electropherograms were detected with UV absorbance at 280 nm. All data analyses were performed using vendor software Compass for iCE (ProteinSimple©). The Compass software aligned each electropherogram using the pI markers so that the x-axis is displayed as a normalized pI for each injection.

### Ac—SINS Assay

AC-SINS method was carried out in a 384-well plate format (Corning® #3702). Initially, 20 nm gold nanoparticles (Ted Pella, Inc., #15705) washed with 0.22 μm filtered Gibco™ Distilled Water were coated with a mixture of capture antibody—80% AffiniPure Goat Anti-Human IgG (H+L) (Jackson ImmunoResearch Laboratories© #109-005-088), and the non-capture antibody—20% ChromPure Goat IgG, whole molecule (Jackson ImmunoResearch Laboratories© #005-000-003), that were initially buffer exchanged into 20 mM sodium acetate pH 4.3 and diluted to 0.4 mg/mL. The mixture of gold nanoparticles, capture antibody and non-capture antibody was incubated in the dark for 18 h at room temperature. Sites unoccupied on the gold nanoparticles were blocked with 1 μM thiolated polyethylene glycol (2 kD) in 20 mM sodium acetate, pH 4.3 to a final concentration of 0.1 μM, followed by 1 h incubation at room temperature. The coated nanoparticles were then concentrated by centrifugation at 21,000×g for 7 min, at 8° C. 95% of the supernatant was removed and the gold pellet was resuspended in the remaining buffer. 5 μL of concentrated nanoparticles were added to 45 μL of antibody at 0.05 mg/mL in Gibco™ PBS pH 7.4 in a 384-well plate. The coated nanoparticles were incubated with the antibody of interest for 4 h at room temperature in the dark. The absorbance was read from 450-700 nm at 1 nm increments, and a Microsoft Excel macro was used to identify the max absorbance, smooth the data, and fit the data using a second-order polynomial. The Δlambda (nm) was calculated based on the smoothed max absorbance of the average blank (PBS alone) subtracted from the smoothed max absorbance of the antibody sample to determine the antibody AC-SINS score. Antibody-antibody interactions directly correlate with the shift in maximum absorbance wavelength of gold nanoparticles coated with the antibody of interest. The cutoff of Δlambda 10 nm was set as high self-aggregation propensity of the antibody.

### NS-ELISA

NS-ELISA was used to measure the propensity of the antibodies to bind to a range of biomolecules to emulate the undesirable non-specific interactions to biological matrices in vivo as described below.

NS-ELISA was carried out in a Corning® 96-well EIA/RIA Easy Wash™ Clear Flat Bottom Polystyrene High Bind Microplate coated overnight at 4° C. with 50 μL of Heparin (Sigma, H₃₁₄₉) diluted with 50 mM sodium carbonate pH 9.6 to a final concentration of 250 μg/mL. The plate was incubated for 2 days at room temperature, wells that were coated with heparin were left uncovered to air dry. Insulin (Sigma-Aldrich®, 19278) and KLH (Sigma-Aldrich®, H₈₂₈₃) were each diluted with 50 mM sodium carbonate pH 9.6 to a final concentration of 5 μg/mL. ssDNA (Sigma-Aldrich®, D8899) and dsDNA (Sigma-Aldrich®, D₄₅₅₃) was diluted with Gibco™ PBS pH7.4 to a final concentration of 10 μg/mL. 50 μL each of insulin, KLH, dsDNA and ssDNA were added to a 96 well plate, followed by the incubation at 37° C. for 2 h. The coating materials were removed, and the plate was blocked with 200 μL of Gibco™ PBS pH7.4, 0.1% Tween®20, and incubated for 1 h at room temperature with shaking at 200 rpm. The plate was washed 3 times with Gibco™ PBS pH7.4, 0.1% Tween 20. 50 μL of each mAb at 100 nM (15 mg/mL) in Gibco™ PBS pH 7.4, 0.1% Tween®20 was added in duplicate to the wells and incubated for 1 h at room temperature with shaking at 200 rpm. Plates were washed three times with Gibco™ PBS pH7.4, 0.1% Tween 20, and 50 μL of 50 ng/mL anti-human IgG HRP (Thermofisher Scientific©, H₁₀₃₀₇) was added to each well. Plates were incubated for 1 h at room temperature, with shaking at 200 rpm. The plate was washed three times with Gibco™ PBS pH7.4, 0.1% Tween 20, and 100 μL of TMB substrate (Cell Signaling Technology©, 7004P6) added to each well. Reactions were stopped after approximately 10 minutes by adding 100 μL of 1 M HCl to each well, and absorbance was read at 450 nm. Binding scores were calculated as the ratio of the ELISA signal of the antibody to the signal of a well containing buffer instead of the primary antibody. The cutoffs considered for each binding molecule (ssDNA. KLH, Insulin, dsDNA and Heparin) were internally calculated.

The results of all three assays are shown in Table 9.1. In these assays a score higher than the cutoff was taken to indicate potentially less desirable biophysical characteristics.

TABLE 11.1
Developability assessment results for anti-GPC3 antibodies

| | | AC- | | | | | |
|---|---|---|---|---|---|---|---|
| | | SINS | | | | | |
| | cIEF | Δlambda | NS-ELISA | | | | |
| Sample | pI | (nm) | ssDNA | KLH | Insulin | dsDNA | Heparin |
| v36180 (M3-H1L1) | 8.96 | 11.7 | 18.60 | 5.45 | 1.78 | 5.93 | 3.07 |
| v37574 (M3-H18L6) | 8.73 | 5.3 | 1.67 | 0.91 | 0.83 | 0.85 | 0.75 |
| v37575 (codrituzumab) | 8.73 | 4.3 | 0.98 | 1.11 | 0.83 | 0.68 | 1.08 |

The pI values determined for the main isoform for the variants v36180, v37574 and codrituzumab (v37575) were 8.96, 8.73 and 8.73 respectively, all within the typical range for therapeutic antibodies. The analysis for v36180 showed a Δlambda higher than 10.0 nm in the AC-SINS assay and higher scores than cutoff on ssDNA, KLH and dsDNA in the NS-ELISA. Antibodies v37574 and v37575 scored below the cutoffs for the AC-SINS assay and NS-ELISA.

### Example 10: Functional Characterization of Anti-GPC3 Antibodies—Binding to Human and Cynomolgus GPC3 on Whole Cells

The cross-reactivity of the humanized anti-GPC3 antibody v37574 (M3-H₁₈L6) to human and cynomolgus monkey GPC3 was assessed by flow cytometry using transfected CHO—S cells as described below. Codrituzumab (v37575) was used as a positive control, and palivizumab (anti-RSV) (v21995) was used as a negative control.

Briefly, CHO—S cells were transiently transfected for −24 hours with a pTT5-based expression plasmid encoding human GPC3 or cynomolgus monkey GPC3, 0.5 μg DNA per 1 million cells, using the Neon™ Transfection System (Thermo Fisher Scientific Corp., Waltham, MA), to transiently express human or cynomolgus monkey GPC3. Following transfection, cells were seeded at 50,000 cells/well in V-bottom 96-well plates and treated with antibody for 24 hours at 4° C. to prevent internalization. Following incubation, cells were washed and stained with anti-human IgG Fc AF647 conjugate (Jackson Immuno Research Labs, West Grove, PA; Cat. No. 109-605-098) at 4° C. for 30 min. Following incubation and washing, fluorescence was detected by flow cytometry on a BD LSRFortessa™ Cell Analyzer (BD Biosciences, Franklin Lake, NJ) with 1,000 minimum events collected per well. AF647/APC-A GeoMean (fluorescence signal geometric mean, proportional to anti-Human AF647 binding) in live cell population was plotted against antibody concentration using GraphPad Prism Version 9 (GraphPad Software, San Diego, CA). The Bmax and Kd values were derived from the “Specific binding with Hill slope” nonlinear regression curve fit in GraphPad Prism Version 9.

The results are shown in Table 10.1. v37574 (M3-H₁₈L6) showed comparable binding to human and cynomolgus GPC3 on CHO—S transfected cells, with apparent Kd values of 534 pM and 376 pM on human GPC3 and cynomolgus monkey GPC3 transfected cells, respectively. The positive control reference antibody v37575 (codrituzumab) showed comparable binding to human and cynomolgus GPC3 on CHO—S transfected cells, with apparent Kd values of 1230 pM and 976 pM on human GPC3 and cynomolgus monkey GPC3 transfected cells, respectively. No binding by non-targeting control v21995 was observed, as expected.

TABLE 10.1
Binding to Cynomolgus Monkey GPC3

| Antibody | Cells | Bmax | Apparent Kd (nM) |
|---|---|---|---|
| v37574 (M3-H18L6) | CHO-S Human | 1264 | 0.53 |
| v37575 (codrituzumab) | GPC3 | 1620 | 1.23 |
| v21995 (palivizumab) | | NA | NA |
| v37574 (M3-H18L6) | CHO-S Cyno | 1002 | 0.38 |
| v37575 (codrituzumab) | GPC3 | 1165 | 0.98 |
| v21995 (palivizumab) | | NA | NA |

### Example 11: Functional Characterization of Anti-GPC3 Antibodies—GPC3 Specificity

The binding cross-reactivity of humanized antibody M3-H₁L1 (v36180) to GPC1, GPC2, GPC3, and GPC5 was assessed by ELISA. For this particular experiment, the M3-H₁L1 antibody used included a C-terminal lysine in the heavy chains. This single amino acid difference has not been shown to affect the activity of this antibody. Anti-GPC3 antibody BMS-986182 (v33624) was included as a GPC3-binding positive control. Palivizumab was included as a non-targeting antibody control. A mouse anti-His tag APC (allophycocyanin)-conjugated antibody (R&D Systems; Cat No. MAB050) was used as a positive control for His tag binding.

Briefly, individual wells of an ELISA 384-well plate was coated with commercial purified soluble His-tagged human GPC1 (ACRO Biosystems; Cat. No. GP1-H₅₂H₉), His-tagged human GPC2 (ACRO Biosystems; Cat. No. GP2-H₅₂H₃), His-tagged human GPC3 (ACRO Biosystems; Cat. No. GP3-H₅₂H₄), or His-tagged human GPC5 (R&D Systems; Cat. No. 2607-G5-050/CF) overnight at 4° C. The plate was blocked with 2% milk in PBS pH 7.4 for 1 hr at RT. Following blocking, primary antibodies were added for 1 hr at RT. Antibody detection was performed by adding HRP-conjugated anti-human IgG F(ab′)2 (Jackson Immuno Research Labs; Cat. No. 109-035-097) or anti-mouse IgG Fc antibodies (Jackson Immuno Research Labs; Cat. No. 115-035-071) for 1 hr at RT. Plates were developed using a 1-Step™ Turbo TMB-ELISA substrate solution (Thermo Scientific; Cat. No. 34022) and HCl was used to stop the reaction. Absorbance was read at 450 nm using a Synergy™ H₁ microplate (BioTek Instruments, Winooski, VT). Absorbance values of each treatment was then subtracted by the absorbance values of blank wells.

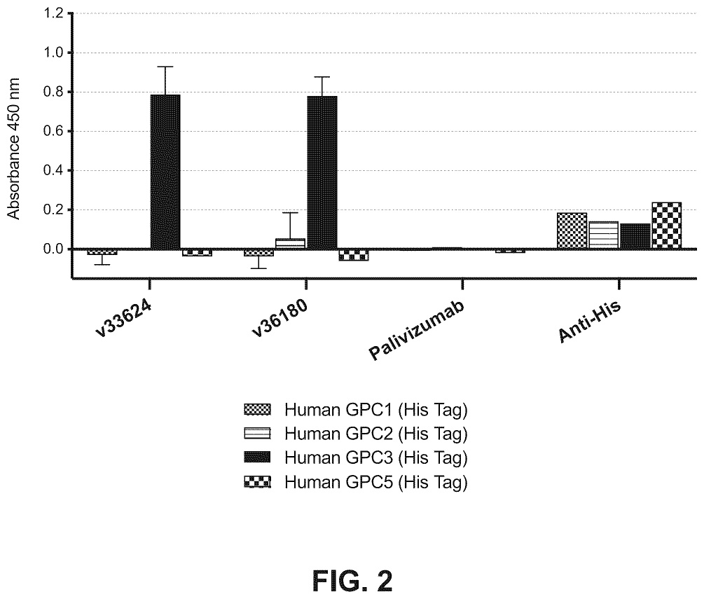

The results are shown in FIG. 2. The anti-His tag antibody demonstrated binding to all soluble His-tagged constructs, demonstrating appropriate loading of target proteins. As shown in FIG. 2, anti-GPC3 antibodies showed expected binding to soluble GPC3 by ELISA, and minimal binding to soluble GPC1, GPC2, and GPC5. Non-targeting palivizumab antibody did not exhibit binding to any of the tested proteins.

### Example 12: Quantification of Surface GPC3 Protein on Tumor Cells

The level of GPC3 expression was assessed in a panel of tumor cell lines using the Quantum™ Simply Cellular anti-human IgG Bead Kit (Bangs Laboratories; Cat. No. 816C).

HepG2, FU-97, Hep3B, JHH-7, JHH-5, Huh-7, NCI-H₄₄₆, Huh-1, Huh-6, PLC/PRF/5, SNU-398, MKN-45, SNU-423, SNU-182, SNU-449, SNU-387, and SNU-601 cells were cultured in 10 cm³ plates at 37° C./5% CO₂ in ATCC-recommended growth media. Tumor cells were detached using Cell Dissociation Buffer (Invitrogen) and incubated with a saturating concentration of reference anti-GPC3 antibody BMS-986182 (v33624) conjugated to AF647 (prepared as described below) for 30 min at 4° C. In parallel, beads calibrated with known antibody binding capacities were similarly stained. Following incubation and washing, fluorescence was detected by flow cytometry using a BD LSRFortessa™ Cell Analyzer (BD Biosciences, Franklin Lake, NJ). The AF647 geometric mean of fluorescence of stained tumor cells and beads were assessed. The antibody binding capacity of each tumor cell was determined by comparison to a standard curve generated with the fluorescence values of the calibrated beads.

For conjugation with Alexa Fluor 647, v33624 was reacted with 8eq. of NHS-Alexa Fluor 647 (Thermofisher A20006, 10 mM) in PBS, pH 7.4. The reaction was allowed to proceed protected from light at room temperature for 150 minutes. Following incubation, the reaction was purified using a 40 kDa Zeba column, pre-equilibrated with PBS pH7.4. Conjugation was confirmed by SEC chromatography (Ex: 650 nm, Em: 665 nm). SEC analysis also estimated the amount of unpurified NHIS-Alexa Fluor 647.

Table 12.1 provides the results of surface GPC3 quantification and identifies tumor cell lines with high, medium, low, or negative expression of the target. Cell lines were designated as “high” expressers if the average number of GPC3 proteins detected was greater than about 1,000,000 receptors per cell; “mid” if the number was between about 50,000 and about 1,000,000 receptors per cell; “low” if the number was between about 500 and about 50,000 receptors per cell; and “negative” if the number was less than about 500 receptors per cell.

TABLE 12.1
GPC3 expression in tumor cell lines

| | Average number of | GPC3 |
|---|---|---|
| Cell Line | GPC3 proteins per cell | Designation |
| HepG2 | 4,060,000 | High |
| FU-97 | 3,430,000 | High |
| Hep3B | 1,270,000 | High |
| JHH-7 | 1,100,000 | High |
| JHH-5 | 380,000 | Mid |
| Huh-7 | 256,000 | Mid |
| NCI-H446 | 228,000 | Mid |
| Huh-1 | 213,000 | Mid |
| Huh-6 | 155,000 | Mid |
| PLC/PRF/5 | 94,600 | Mid |
| SNU-398 | 52,800 | Mid |
| MKN-45 | 41,100 | Low |
| SNU-423 | 2,340 | Low |
| SNU-182 | 1,130 | Low |
| SNU-449 | 567 | Low |
| SNU-387 | <500 | Negative |
| SNU-475 | NT* | Negative |
| SNU-601 | <500 | Negative |

*NT = Not Tested;
SNU-475 is a GPC3-negative cell line as characterized by Sung et al. (Sung et al. 2003. Cancer Sci 94(3):259-62)

### Example 13: Functional Characterization of Anti-GPC3 Antibodies—Cellular Binding

The on-cell binding capabilities of the humanized variants v36180 (M3-H₁L1) and v37574 (M3-H₁₈L6) were assessed on HepG2 (hepatocellular carcinoma; GPC3-high) and JHH-7 (hepatocellular carcinoma; GPC3-high) by flow cytometry as described below. The GPC3-targeting antibody codrituzumab (v37575) was used as a positive control, and anti-RSV antibody palivizumab (v22277) was used as a negative control. Variant 22277 differs from anti-RSV antibody v21995 used in previous examples in that it has a heterodimeric Fc. This does not affect the function of this antibody.

Briefly, cells were seeded at 50,000 cells/well in V-bottom 96-well plates and treated with antibody for 24 hours at 4° C. to prevent internalization. Following incubation, cells were washed and stained with anti-Human IgG Fc AF647 conjugate (Jackson Immuno Research Labs, West Grove, PA; Cat. No. 109-605-098) at 4° C. for 30 min. Following incubation and washing, fluorescence was detected by flow cytometry on a BD LSRFortessa™ Cell Analyzer (BD Biosciences, Franklin Lake, NJ) with 1,000 minimum events collected per well. AF647/APC-A GeoMean (fluorescence signal geometric mean, proportional to anti-Human AF647 binding) in live cell population was plotted against antibody concentration using GraphPad Prism Version 9 (GraphPad Software, San Diego, CA). The Bmax, Curve Hill Slope (h) and Kd values were derived from the “Specific binding with Hill slope” nonlinear regression curve fit in GraphPad Prism Version 9.

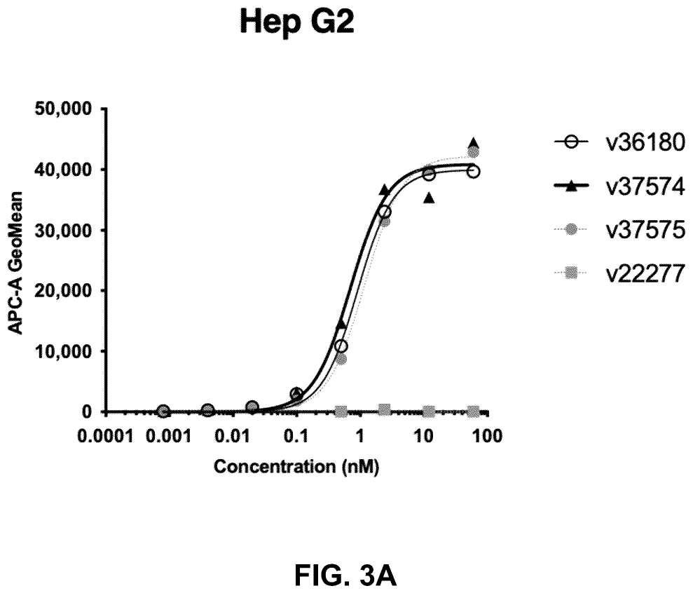

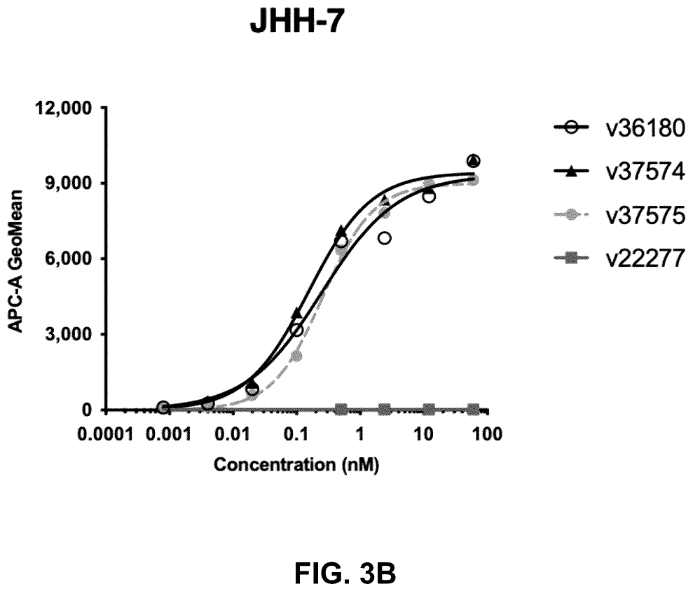

The results are shown in Table 13.1, and FIG. 3A and FIG. 3B. Both v36180 and v37574 yielded comparable apparent Kd and Bmax values to v37575 in both HepG2 and JHH-7.

TABLE 13.1
Cellular Binding of anti-GPC3 antibodies

| | HepG2 | | | JHH-7 | | |
|---|---|---|---|---|---|---|
| | | Curve Hill | Kd | | Curve Hill | Kd |
| Antibody | Bmax | Slope (h) | (nM) | Bmax | Slope (h) | (nM) |
| v36180 (M3-HIL1) | 39,964 | 1.54 | 0.91 | 9,339 | 0.73 | 0.25 |
| v37574 (M3-H18L6) | 33,929 | 1.41 | 1.10 | 8,524 | 1..09 | 0.22 |
| v37575 (codrituzumab) | 42,286 | 1.48 | 1.20 | 9,000 | 1.09 | 0.26 |
| v22277 (palivizumab) | NA | NA | >60 | NA | NA | >60 |

### Example 14: Functional Characterization of Anti-Gpc3 Antibodies—Internalization

The receptor-mediated internalization capabilities of the humanized variants, v36180 (M3-H₁L1) and v37574 (M3-H₁₈L6), in GPC3-expressing cell lines Hep G2 (high GPC3-expressing) and JHH-7 (high GPC3-expressing) were determined by flow cytometry as described below. The GPC3-targeting antibody codrituzumab (v37575) was used as a positive control, and the anti-RSV antibody palivizumab (v22277) was used as a negative control.

Briefly, antibodies were fluorescently labeled by coupling to an anti-Human IgG Fc Fab fragment AF488 conjugate (Jackson Immuno Research Labs, West Grove, PA; Cat. No. 109-547-008) at a 1:1 stoichiometric molar ratio in PBS pH 7.4 (Thermo Fisher Scientific, Waltham, MA; Cat. No. 10010-023), for 24 hours at 4° C. Cells were seeded at 50,000 cells/well in 48-well plates and incubated overnight under standard culturing conditions (37° C./5% CO₂) in Gibco™ Dulbecco's Modified Eagle Media (DMEM) (Thermo Fisher Scientific, Waltham, MA; Cat. No. 11995040) with 10% Fetal Bovine Serum (FBS) (Thermo Fisher Scientific, Waltham, MA; Cat. No. 12483020) for Hep G2 cells, and Gibco™ William's E Medium, GlutaMAX™ Supplement (Thermo Fisher Scientific, Waltham, MA; Cat. No. 32551020) with 10% FBS (Thermo Fisher Scientific, Waltham, MA; Cat. No. 12483020) for JHH-7 cells. Coupled antibodies were added to cells the following day at 10 nM or at 100 nM and incubated under standard culturing conditions for 5-24 hours to allow for internalization. Following incubation, cells were dissociated, washed, and surface AF488 fluorescence was quenched using an anti-AF488 antibody (Life Technologies, Carlsbad, CA; Cat. No. A-11094) at 100 nM for 30 minutes at 4° C. Quenched AF488 fluorescence (internalized fluorescence) was detected by flow cytometry on a BD LSRFortessa™ Cell Analyzer (BD Biosciences, Franklin Lake, NJ) with 1,000 minimum events collected per well. The AF488/FITC-A GeoMean (fluorescence signal geometric mean, proportional to anti-Human Fab AF488 labelling) was calculated for the live single cell population using FlowJo™ Version 10.8.1 (BD Biosciences, Franklin Lake, NJ) and plotted for each variant using GraphPad Prism Version 9 (GraphPad Software, San Diego, CA).

The results for the 10 nM test concentration are shown in Table 14.1 and are similar to those observed for the 100 nM test concentration. GPC3-targeting antibody v37575 positive control showed comparable levels of internalization to the humanized variants v36180 and v37574 in both Hep G2 cells (high GPC3) and JHH-7 cells (high GPC3). In both Hep G2 and JHH-7 cells, both humanized antibodies v36180 and v37574, and v37575 positive control showed increased internalization compared to palivizumab negative control across all tested concentrations (100 nM and 10 nM) and time points (5-24 hours). For example, following a 5-hour incubation in Hep G2 cells, humanized variants v36180 and v37574 showed 17.0- and 16.3-fold increase in internalized fluorescence compared to palivizumab, respectively at 10 nM. Following a 24-hour incubation in Hep G2 cells, humanized variants v36180 and v37574 showed 66.2- and 74.1-fold increase in internalized fluorescence compared to palivizumab, respectively, at 10 nM.

TABLE 14.1
Internalization of anti-GPC3 antibodies at 10 nM

| | Hep G2 (fold increase | | JHH-7 (fold increase | |
|---|---|---|---|---|
| | over palivizumab) | | over palivizumab) | |
| Antibody (10 nM) | 5 hours | 24 hours | 5 hours | 24 hours |
| v36180 (M3-H1L1) | 17.0 | 66.2 | 8.6 | 46.4 |
| v37574 (M3-H18L6) | 16.3 | 74.1 | 8.8 | 46.1 |
| v37575 (codrituzumab) | 14.4 | 57.2 | 8.5 | 46.7 |
| v22277 (palivizumab) | 1.0 | 1.0 | 1.0 | 1.0 |
| Blank | 0.9 | 0.8 | 1.0 | 0.8 |

### Example 15: Preparation of Antibody-Drug Conjugates

Antibody-drug conjugates shown in Table 15.1 were prepared. Exemplary protocols are provided below.

### v37574-MC-GGFG-AM-DXd1, v37574-MC-GGFG-Compound 141, v37574-MC-GGFG-AM-Compound 139 DAR 8

A solution (3.88 mL) of variant v37574 (28.5 g) in 50 mM histidine buffer, pH 6.0 was diluted in 0.481 mL of PBS, pH 7.4. The antibody was reduced by addition of 5 mM diethylenetriamine pentaacetic acid (DTPA) (1.14 mL in PBS, pH adjusted to 7.4) and 10 mM of an aqueous tris(2-carboxyethyl)phosphine (TCEP) solution (0.197 mL, 10 eq.). After 3 hours at 37° C., the reduced antibody was purified using Zeba™ desalting columns (40 kDa MWCO, 10 mL; Thermo Scientific, 87772) primed with 10 mM NaOAc, pH 5.5. To the antibody solution was added 340 μL of DMSO and an excess of drug-linker MC-GGFG-AM-DXd1, MC-GGFG-Compound 141 or MC-GGFG-AM-Compound 139 (295 μL; 15 eq.) from a 10 mM DMSO stock solution. The conjugation reaction proceeded at room temperature with mixing for 60 minutes. An excess of a 30 mM N-acetyl-L-cysteine solution (79 μL, 12 eq.) was added to quench the conjugation reaction.

### v37574-MC-GGFG-Compound 141, v37574-MC-GGFG-AM-Compound 139 DAR 4

A solution (2.66 mL) of humanized variant v37574 (20 mg) in 50 mM histidine buffer, pH 6.0 was diluted in 0.768 mL of PBS, pH 7.4. The antibody was reduced by addition of 5 mM diethylenetriamine pentaacetic acid (DTPA) (0.87 mL in PBS, pH adjusted to 7.4) and 10 mM of an aqueous tris(2-carboxyethyl)phosphine (TCEP) solution (0.042 mL, 3.15 eq.). After 120 minutes at 37° C., to the antibody solution was added 347 μL of DMSO and an excess of either MC-GGFG-Compound 141, MC-GGFG-AM-Compound 139 (134 μL; 10 eq.) from a 10 mM DMSO stock solution. The conjugation reaction proceeded at room temperature with mixing for 60 minutes. An excess of 30 mM N-acetyl-L-cysteine solution (40 μL, 9 eq.) was added to quench each conjugation reaction.

TABLE 15.1
Antibody-Drug Conjugates

| Variant | Antibody | Drug-Linker¹ | DAR |
|---|---|---|---|
| v33624 | Humanized | MC-GGFG-AM-DXd1 | 8 |
| (BMS-986182) | | | |
| v36180 | Humanized | MC-GGFG-AM-DXd1 | 8 |
| (M3-HIL1) | | MC-GGFG-Compound 141 | 8 |
| | | MC-GGFG-AM-Compound 139 | 8 |
| | | MC-GGFG-AM-Compound 141 | 8 |
| | | MT-GGFG-Compound 140 | 8 |
| | | MT-GGFG-Compound 141 | 8 |
| v37574 | Humanized | MC-GGFG-AM-DXd1 | 8 |
| (M3-H18L6) | | MC-GGFG-Compound 141 | 8 |
| | | MC-GGFG-AM-Compound 139 | 8 |
| | | MC-GGFG-AM-DXd1 | 4 |
| | | MC-GGFG-Compound 141 | 4 |
| | | MC-GGFG-AM-Compound 139 | 4 |
| v37575 | Humanized | MC-GGFG-AM-DXd1 | 8 |
| (codrituzumab) | | MC-GGFG-AM-DXd1 | 4 |

Controls

| | | | |
|---|---|---|---|
| v21995 | — | MC-GGFG-AM-DXd1 | 8 |
| (palivizumab) | | MC-GGFG-Compound 141 | 8 |
| | | MC-GGFG-AM-Compound 139 | 8 |
| | | MT-GGFG-Compound 140 | 8 |
| | | MT-GGFG-Compound 141 | 8 |
| | | MC-GGFG-Compound 141 | 4 |
| | | MC-GGFG-AM-Compound 139 | 4 |

### Example 16: Purification and Characterization of Antibody-Drug Conjugates

ADCs prepared as described in Example 15 were purified on an AKTA™ pure chromatography system (Cytiva Life Sciences, Marlborough, MA) using a 53 mL HiPrep 26/10 Desalting column (Cytiva Life Sciences, Marlborough, MA) and a mobile phase consisting of 10 mM NaOAc, pH 4.5 with 150 mM NaCl and a flow rate of 7.5 mL/min.

Following purification, the concentration of the ADCs was determined by measurement of absorption at 280 nm using extinction coefficients taken from the literature (European Patent No. 3 342 785, for MC-GGFG-AM-DXd1) or determined experimentally (for the remaining drug-linkers). ADCs were also characterized by hydrophobic interaction chromatography (HIC) and size exclusion chromatography (SEC) as described below.

### 16.1 Hydrophobic Interaction Chromatography

Antibody and ADCs were analyzed by HIC to estimate the drug-to-antibody ratio (DAR). Chromatography was performed on an Agilent Infinity 111290 HPLC (Agilent Technologies, Santa Clara, CA) using a TSKgel® Butyl-NPR column (2.5 μm, 4.6×35 mm; TOSOH Bioscience GmbH, Griesheim, Germany) and employing a gradient of 95/5% MPA/MPB to 5/95% MPA/MPB over a period of 12 minutes at a flow rate of 0.5 mL/min (MPA=1.5 M (NH₄)₂SO₄, 25 mM NaₓPO₄, pH 7 and MPB=75% 25 mM NaₓPO₄, pH 7, 25% isopropanol). Detection was by absorbance at 280 nm.

### 16.2 Size Exclusion Chromatography

The extent of aggregation of the antibody and ADCs (˜15-150 μg, 5 μL injection volume) was assessed by SEC on an Agilent Infinity 111260 HPLC (Agilent Technologies, Santa Clara, CA) using an AdvanceBio SEC column (300 angstroms, 2.7 μm, 7.8×150 mm) (Agilent, Santa Clara, California) and a mobile phase consisting of 150 mM phosphate, pH 6.95 and a flow rate of 1 mL/min. Detection was by absorbance at 280 nm.

### Results

The individual contributions of the DAR0, DAR2, DAR4, DAR6 and DAR8 species to the average DAR of the purified ADCs were assessed by integration of the HPLC-HIC chromatogram. The average drug to antibody ratio (DAR) of each ADC was determined by the weighted average of each DAR species. The average DAR for each ADC, when rounded to the nearest integer, was the same as the target DAR shown in Table 16.1.

The extent of aggregation and monomer content was assessed by integration of the HPLC-SEC chromatogram. The monomer peak of each ADC was identified as the peak with the same retention time as the unconjugated antibody from which each ADC was derived from. All peaks with an earlier retention time relative to the monomer species was determined to be aggregated species. Percent monomer species determined for each ADC is shown in Table 16.1. All ADC preparations showed >95% monomer species.

TABLE 16.1
Characterization of Prepared ADCs

| | | Target | % |
|---|---|---|---|
| Variant | Drug-Linker | DAR | Monomer |
| v33624 | MC-GGFG-AM-DXd1 | 8 | 100 |
| (BMS-986182) | | | |
| v36180 | MC-GGFG-AM-DXd1 | 8 | 100 |
| (M3-H1L1) | MC-GGFG-Compound 141 | 8 | 100 |
| | MC-GGFG-AM-Compound 139 | 8 | 99.7 |
| | MC-GGFG-AM-Compound 141 | 8 | 99.2 |
| | MT-GGFG-Compound 140 | 8 | 100 |
| | MT-GGFG-Compound 141 | 8 | 100 |
| v37574 | MC-GGFG-AM-DXd1 | 8 | 100 |
| (M3-H18L6) | MC-GGFG-Compound 141 | 8 | 100 |
| | MC-GGFG-AM-Compound 139 | 8 | 100 |
| | MC-GGFG-AM-DXd1 | 4 | 100 |
| | MC-GGFG-Compound 141 | 4 | 100 |
| | MC-GGFG-AM-Compound 139 | 4 | 100 |
| v37575 | MC-GGFG-AM-DXd1 | 8 | 100 |
| (codrituzumab) | MC-GGFG-AM-DXd1 | 4 | 100 |
| v21995 | MC-GGFG-AM-DXd1 | 8 | 100 |
| (palivizumab) | MC-GGFG-Compound 141 | 8 | 97.2 |
| | MC-GGFG-AM-Compound 139 | 8 | 100 |
| | MT-GGFG-Compound 140 | 8 | 98.8 |
| | MT-GGFG-Compound 141 | 8 | 100 |
| | MC-GGFG-Compound 141 | 4 | 100 |
| | MC-GGFG-AM-Compound 139 | 4 | 100 |

### Example 17: In Vitro Cytotoxicity of Anti-GPC3 Antibody-Drug Conjugates—2D Monolayer Cell Culture

The cell growth inhibition (cytotoxicity) capabilities of the humanized variants M3-H₁L1 (v36180) and M3-H18L6 (v37574) conjugated to various drug-linkers at different DARs were assessed in a panel of GPC3-expressing cell lines as described below. An ADC comprising the anti-GPC3 antibody codrituzumab (v37575) conjugated to DXd1 was assessed as a comparator. Cell lines used were GPC3-high cells: HepG2 (hepatocellular carcinoma), JHH-7 (hepatocellular carcinoma), and Hep3B (hepatocellular carcinoma); GPC3-mid cells: JHH-5 (hepatocellular carcinoma), NCI-H446 (lung carcinoma), Huh-1 (hepatocellular carcinoma), Huh-6 (hepatoblastoma), Huh-7 (hepatocellular carcinoma), and PLC/PRF/5 (hepatocellular carcinoma); GPC3-low cells: SNU-398 (hepatocellular carcinoma), SNU423 (hepatocellular carcinoma), SNU-182 (hepatocellular carcinoma), and SNU-449 (hepatocellular carcinoma); and GPC3-negative cells: SNU-387 (hepatocellular carcinoma) and SNU-601 (gastric carcinoma). SNU-475 hepatocellular carcinoma cells were also included as a potential GPC3-negative cell based on mRNA expression. ADCs comprising the antibody palivizumab (v21995) were used as non-targeted controls. In these experiments, the ability of the GPC3-targeting ADCs to specifically kill GPC3 expressing cells was assessed as was the difference in potency between ADCs having DAR8 or DAR4.

Briefly, cells were seeded in 384-well plates at 1,000 cells/well and treated with a titration of test article, generated in RPMI-1640 (Thermo Fisher Scientific; Cat. No. 15230-162)+10% FBS (Thermo Fisher Scientific; Cat. No. 12483-020). Cells were incubated for 4 days in RPMI-1640+10% FBS at 37° C./5% CO₂. After incubation, CellTiter-Glo® reagent (Promega Corporation, Madison, WI) was added to all wells and luminescence corresponding to ATP present in each well was measured using a Synergy™ H₁ plate reader (BioTek Instruments, Winooski, VT). The % cytotoxicity value for each treatment was calculated by the following formula: (1−(Luminescence of Treated Cells/Average Luminescence of Untreated Cells))×100. These values were plotted against test article concentration using GraphPad Prism 9 software (GraphPad Software, San Diego, CA).

The cytotoxic activity of DAR8 ADCs is shown in Table 17.1.

TABLE 17.1
Cytotoxicity of DAR8 ADCs (2D monolayer cell culture)

| | Cell Line (GPC3 Expression) | | | | |
|---|---|---|---|---|---|
| | HepG2 | JHH-7 | JHH-5 | Huh-6 | Huh-7 |
| | (High) | (High) | (Mid) | (Mid) | (Mid) |
| | EC50 (nM) | | | | |
| v37574-MC-GGFG- | 0.06 | 0.08 | 0.29 | IC | 0.03 |
| AM-DXd1 | | | | | |
| v37575-MC-GGFG- | 0.16 | 0.10 | 0.18 | 0.30 | 0.03 |
| AM-DXd1 | | | | | |
| v36180-MC-GGFG- | 0.13 | IC* | 0.16 | 0.52 | 0.04 |
| AM-DXd1 | | | | | |
| v36180-MC-GGFG- | 0.09 | 1.00 | 0.14 | IC | 0.01 |
| AM-Compound 139 | | | | | |
| v36180-MC-GGFG- | 0.03 | 0.03 | 0.11 | 0.12 | 0.03 |
| Compound 141 | | | | | |
| v36180-MC-GGFG- | 0.03 | 0.02 | 0.08 | 0.12 | 0.01 |
| AM-Compound 141 | | | | | |
| v21995-MC-GGFG- | 23.94 | 14.66 | 17.41 | IC | 3.31 |
| AM-DXd1 | | | | | |

*IC = Incomplete Curve, an accurate EC50 cannot be determined

As shown in Table 17.1, targeted killing as evidenced by enhanced potency relative to a non-targeting DXd1 ADC, was observed in GPC3-high HepG2 and JHH-7, but not in GPC3-high Hep3B cells. In GPC3-mid lines targeted killing was observed in JHH-5, Huh-6, and Huh-7 cells but not in GPC3-mid Huh-1 and PLC/PRF/5 cell lines. Anti-GPC3 DXd1 ADCs did not show targeted killing in all GPC3-negative and GPC3-low cell lines tested. DXd1 conjugates of codrituzumab (v37575), M3-H₁L1 (v36180), or M3-H18L6 (v37574) demonstrated comparable cytotoxic properties across the cell lines tested. In addition, MC-GGFG-AM-Compound 139, MC-GGFG-Compound 141, and MC-GGFG-AM-Compound 141 conjugates of M3-H₁L1 (v36180) exhibited comparable cytotoxicity to the MC-GGFG-AM-DXd1 conjugate. These data show that anti-GPC3 DXd1 ADCs exhibited dose-dependent killing in certain hepatocellular or hepatoblastoma cell lines.

Table 17.2 shows data comparing potency between ADCs having DAR8 or DAR4 in selected cell lines where targeted killing was observed.

TABLE 17.2
Comparison of Cytotoxicity between ADCs of DAR4 and DAR8

| | | EC50 (nM) | | |
|---|---|---|---|---|
| ADC | DAR | HepG2 | JHH-7 | JHH-5 |
| v37574-MC-GGFG- | 4.0 | 1.12 | 2.10 | IC* |
| AM-Compound 139 | | | | |
| v37574-MC-GGFG- | 8.0 | 0.45 | 0.56 | 0.76 |
| AM-Compound 139 | | | | |
| v37574-MC-GGFG- | 3.8 | 0.09 | 0.50 | 4.00 |
| Compound 141 | | | | |
| v37574-MC-GGFG- | 8.0 | 0.05 | 0.13 | 0.30 |
| Compound 141 | | | | |

Controls

| | | | | |
|---|---|---|---|---|
| v21995-MC-GGFG- | 4.1 | IC | >100 | IC |
| AM-Compound 139 | | | | |
| v21995-MC-GGFG- | 8.0 | IC | >100 | IC |
| AM-Compound 139 | | | | |
| v21995-MC-GGFG- | 4.2 | IC | >100 | >100 |
| Compound 141 | | | | |
| v21995-MC-GGFG- | 8.0 | IC | >100 | IC |
| Compound 141 | | | | |

*IC = Incomplete Curve where an accurate EC50 cannot be determined

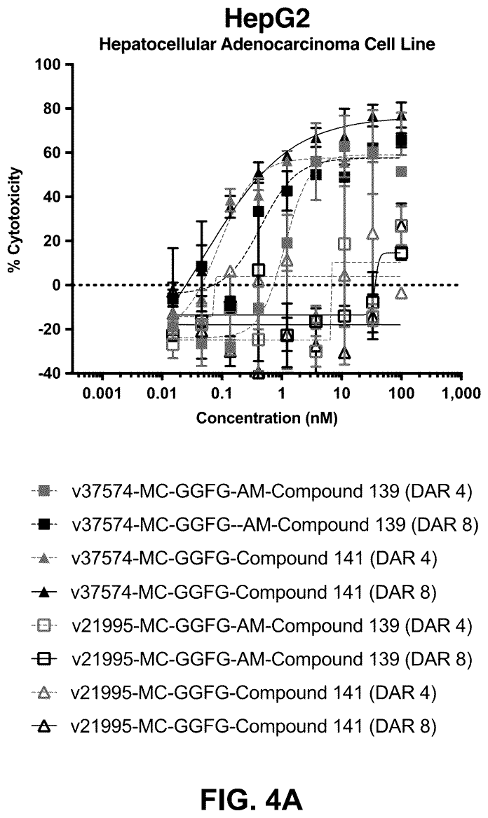

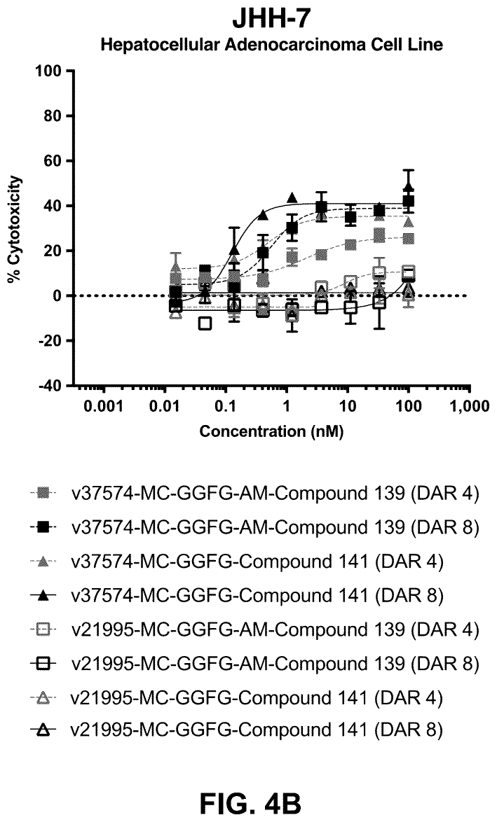

As shown in Table 17.2, ADCs of v37574 conjugated to MC-GGFG-AM-Compound 139 or MC-GGFG-Compound 141 demonstrated potent dose-dependent cytotoxicity relative to non-targeting controls in GPC3-high HepG2 and JHH-7 cells, as well as GPC3-mid JHH-5 cells (representative curves shown in FIG. 4A (HepG2 cells) and FIG. 4B (JHH-7 cells)). Regardless of drug-linker, DAR8 ADCs consistently exhibited stronger cytotoxicity compared to DAR4 ADCs.

### Example 18: In Vitro Cytotoxicity of Anti-GPC3 Antibody-Drug Conjugates—3D Spheroid Cell Culture

The cytotoxicity of the humanized variant M3-H18L6 (v37574) conjugated to various drug-linkers was assessed in a panel of 3D spheroids of GPC3-expressing cell lines as described below. Cell lines used were GPC3-high HepG2 (hepatocellular carcinoma), GPC3-mid NCI-H446 (lung carcinoma) cells, and GPC3-negative SNU-601 (gastric carcinoma) cells. ADCs comprising the antibody palivizumab (v21995) were used as non-targeted controls.

Briefly, cells were seeded in Ultra-Low Attachment 384-well plates, centrifuged and incubated at 37° C./5% Co₂ for 4 days in ATCC-recommended complete growth medium to allow for spheroid formation and growth. Monoculture cell line spheroids were then treated with a titration of test article, generated in cell growth medium RPMI-1640 (Thermo Fisher Scientific; Cat. No. 15230-162)+10% FBS (Thermo Fisher Scientific; Cat. No. 12483-020). Spheroids were incubated for a further 6 days. After incubation, CellTiter-Glo® 3D reagent (Promega Corporation, Madison, WI) was added in all wells. Plates were incubated in the dark at room temperature for 1 hour and luminescence was quantified using a BioTek Cytation 5 Cell Imaging Multi-Mode Reader (Agilent Technologies, Inc., Santa Clara, CA). The % cytotoxicity value for each treatment was calculated by the following formula: (1−(Luminescence of Treated Cells/Average Luminescence of Untreated Cells))×100. These values were plotted against test article concentration using GraphPad Prism 9 software (GraphPad Software, San Diego, CA).

The results are provided in Table 18.1.

TABLE 18.1
Cytotoxicity of ADCs in 3D Spheroid Cell Culture

| | | EC50 (nM) | | |
|---|---|---|---|---|
| ADC | DAR | HepG2 | NCI-H446 | SNU-601 |
| v37574-MC-GGFG- | 4.0 | 0.55 | 0.31 | >100 |
| AM-Compound 139 | | | | |
| v37574-MC-GGFG- | 8.0 | 0.32 | 0.07 | 34.54 |
| AM-Compound 139 | | | | |
| v37574-MC-GGFG- | 3.8 | 0.11 | 0.15 | 53.59 |
| Compound 141 | | | | |
| v37574-MC-GGFG- | 8.0 | 0.22 | 0.06 | 44.00 |
| Compound 141 | | | | |

Controls

| | | | | |
|---|---|---|---|---|
| v21995-MC-GGFG- | 4.1 | IC* | >100 | 56.45 |
| AM-Compound 139 | | | | |
| v21995-MC-GGFG- | 8.0 | IC | 30.62 | 29.48 |
| AM-Compound 139 | | | | |
| v21995-MC-GGFG- | 4.2 | IC | 44.90 | 96.92 |
| Compound 141 | | | | |
| v21995-MC-GGFG- | 8.0 | IC | 22.17 | 35.82 |
| Compound 141 | | | | |

*IC = Incomplete curve where an accurate EC50 cannot be determined

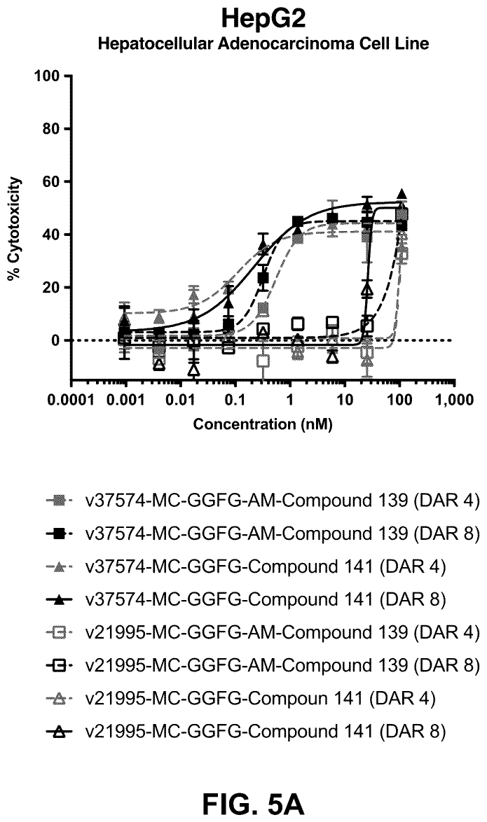

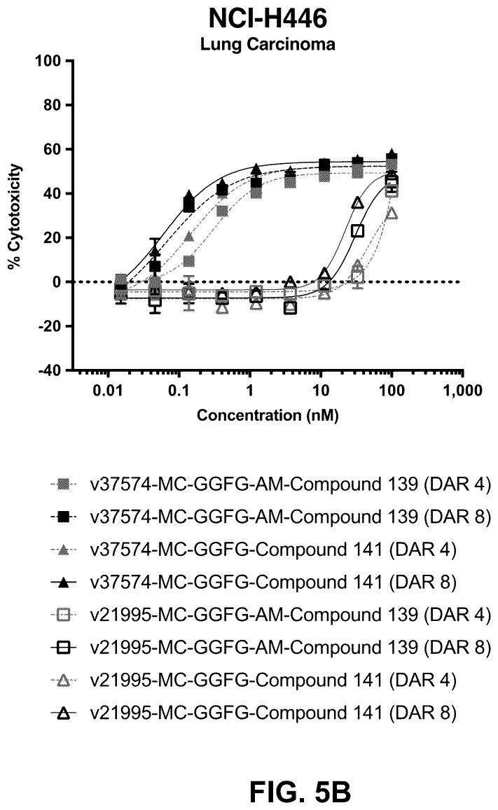

As shown in Table 18.1, ADCs of v37574 conjugated to MC-GGFG-AM-Compound 139 or MC-GGFG-Compound 141 demonstrated potent dose-dependent cytotoxicity in GPC3-high HepG2 and JHH-7 spheroids, as well as GPC3-mid NCI-H446 spheroids compared to non-targeting controls (representative curves shown in FIG. 5A (HepG2 cells) and FIG. 5B (NCI-H₄₄₆)). Targeted killing was not observed in GPC3-negative SNU-601 cells. Compound 139 DAR8 ADCs exhibited higher potencies vs Compound 139 DAR4 ADCs in GPC3-positive tumor spheroids.

### Example 19: In Vitro Cytotoxicity of Anti-GPC3 Antibody-Drug Conjugates—2D Monolayer and 3d Spheroid Cell Cultures

The cell growth inhibition (cytotoxicity) capabilities of the humanized antibody BMS-986182 (v33624) and humanized variant M3-H₁L1 (v36180) conjugated to various drug-linkers were assessed in a panel of GPC3-expressing cell lines as described below. Cell lines used were GPC3-high FU-97 (gastric carcinoma), JHH-7 (hepatocellular carcinoma), and Hep3B (hepatocellular carcinoma), GPC3-mid NCI-H446 (lung carcinoma), Huh-6 (hepatoblastoma), and Huh-7 (hepatocellular carcinoma), and GPC3-low MKN-45 (gastric carcinoma) cells. ADCs of the antibody palivizumab (v21995) were used as non-targeted controls.

2D cytotoxicity assays were carried out as described in Example 17.

3D spheroid cytotoxicity assays were carried out as described in Example 18, except spheroids were incubated with test article for 7 days rather than 6.

The results for the 2D monolayer cell culture cytotoxicity assays are shown in Table 19.1 below.

TABLE 19.1
Cytotoxic Potency of ADCs in 2D monolayer cell culture

| | | 2D EC50 (nM) | | | | | |
|---|---|---|---|---|---|---|---|
| | | | | NCI- | | | |
| ADC | DAR | FU-97 | JHH-7 | H446 | Huh-6 | Huh-7 | MKN-45 |
| v33624-MC-GGFG-AM-DXd1 | 7.6 | 0.07 | 0.37 | 0.01 | 0.07 | 0.08 | >50 |
| v36180-MC-GGFG-AM-DXd1 | 7.6 | 0.06 | 0.32 | 0.01 | 0.05 | 0.06 | >50 |
| v36180-MT-GGFG-Compound 140 | 7.7 | 1.32 | 0.88 | IC* | 1.04 | 0.67 | >50 |
| v36180-MT-GGFG-Compound 141 | 7.7 | 0.17 | 0.33 | 0.01 | 0.20 | 0.03 | >50 |
| v21995-MC-GGFG-AM-DXd1 | 8.0 | 9.58 | 71.70 | 1.38 | 16.45 | 4.35 | >50 |

*IC = Incomplete Curve where an accurate EC50 cannot be determined

*IC=Incomplete Curve where an accurate EC50 cannot be determined

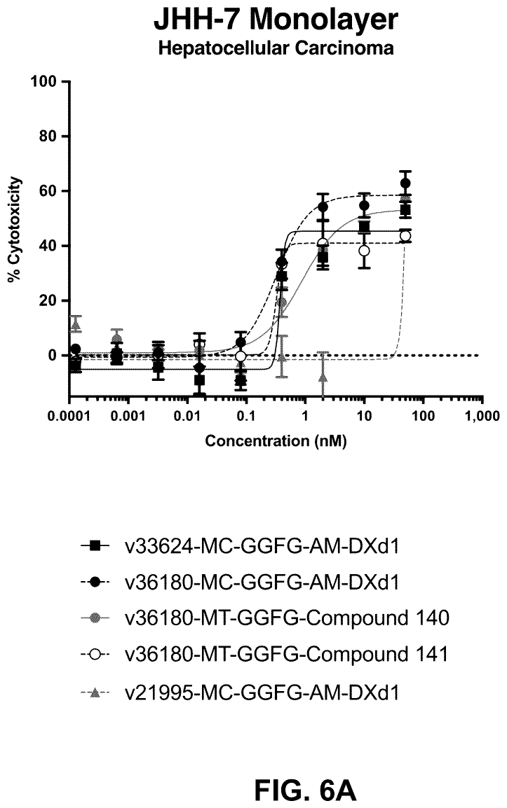

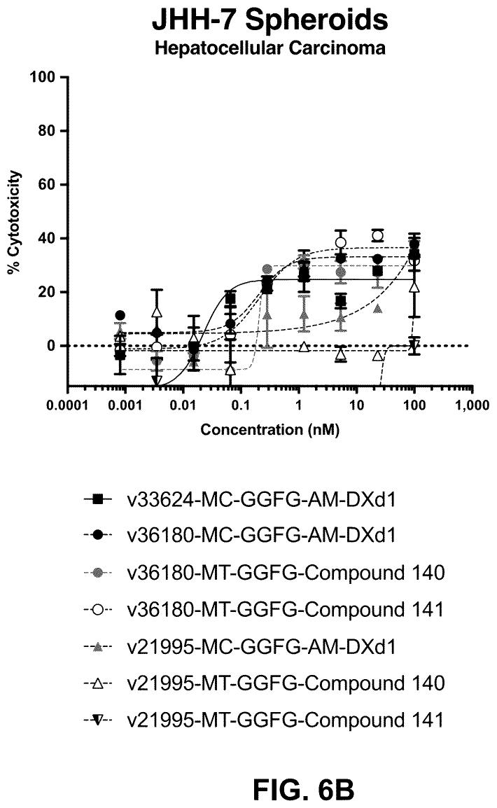

Anti-GPC3 DXd1 ADCs exhibited dose-dependent killing in a panel of cell lines in 2D cytotoxicity assays. As shown in Table 19.1, targeted killing by anti-GPC3 DXd1 ADCs as evidenced by enhanced potency relative to a non-targeting DXd1 ADC, was observed in GPC3-high cells FU-97 and JHH-7 cells (representative curves for JHH-7 cells are shown in FIG. 6A), but not in GPC3-high Hep3B cells. Targeted killing was also observed in GPC3-mid NCI-H₄₄₆, Huh-7, and Huh-6 cell lines, but not in GPC3-low MKN-45 cells. The cytotoxic effect was comparable in DXd1 ADCs of v33624 and v36180. v36180-MT-GGFG-Compound 141 displayed comparable cytotoxicity to v36180-MC-GGFG-AM-DXd1, with a slight potency reduction in FU-97 and Huh-7 cells, whereas v36180-MT-GGFG-Compound 140 demonstrated weaker potency compared to its DXd1 counterpart in all cell lines tested.

The results for the 3D cytotoxicity assay are shown in Table 19.2 below.

TABLE 19.2
Cytotoxic Potency of ADCs in 3D spheroid cell culture

| | | EC50 (nM) | |
|---|---|---|---|
| ADC | DAR | JHH-7 | NCI-H446 |
| v33624-MC-GGFG-AM-DXd1 | 7.6 | 0.02 | 0.01 |
| v36180-MC-GGFG-AM-DXd1 | 7.6 | 0.20 | 0.02 |
| v36180-MT-GGFG-Compound 140 | 7.7 | 0.21 | 0.05 |
| v36180-MT-GGFG-Compound 141 | 7.7 | 0.21 | 0.04 |
| v21995-MC-GGFG-AM-DXd1 | 8.0 | IC* | 2.37 |
| v21995-MT-GGFG-Compound 140 | 8.0 | >100 | 9.06 |
| v21995-MT-GGFG-Compound 141 | 8.0 | >100 | 9.35 |

*IC = Incomplete Curve where an accurate EC50 cannot be determined

The data in Table 19.2 demonstrate that anti-GPC3 ADCs showed targeted dose-dependent killing of GPC3-high JHH-7 and GPC3-mid NCI-H446 3D spheroids compared to non-targeted ADCs (representative curves for JHH-7 cells are shown in FIG. 6B). Regardless of drug-linker, all ADCs of v36180 demonstrated comparable potency in both cell lines. v33624-MC-GGFG-AM-DXd1 elicited slightly increased cytotoxic potency compared to v36180-MC-GGFG-AM-DXd1 in JHH-7 cells.

### Example 20: Stability of ADCs in Mouse Plasma

The in vitro stability in mouse plasma of 4 ADCs comprising the variant v36180 and variant v33624 was assessed using immunoprecipitation/mass spectrometry (IP-MS) as described below. The ADCs assessed were: v36180-MC-GGFG-AM-DXd1 (DAR8), v36180-MT-GGFG-Compound 140 (DAR8), v36180-MT-GGFG-Compound 141 (DAR8), and v33624-MC-GGFG-AM-DXd1 (DAR8). The assay was carried out as described below.

ADCs were diluted to reach a final concentration of 1 mg/mL in CD₁ mouse plasma from BioreclamationIVT™ (Catalog #MSE00PL38NCXNN). Samples were then incubated at 37° C. for a duration of 7 days. Samples were taken at various timepoints (0, 0.17, 1 and 7 days) and immediately frozen at −80° C. before proceeding with analysis by IP-MS.

IP-MS was performed as follows. Briefly, for each sample, 15 μg of biotinylated anti-human IgG F(ab′)2 antibody from Jackson ImmunoResearch™ (Catalog #109-065-097) was coupled to magnetic beads coated with streptavidin from GE Healthcare Biosciences™ (Catalog #28-9857-99) for 30 min at room temperature. Following coupling, the beads were incubated with test sample for 1.5 hrs at room temperature to allow for immunocapture. Following incubation and washing using DynaMag™-2 magnet (ThermoFisher Scientific™ Corporation, Waltham, MA), immunocaptured sample was reduced using 50 μL of dithiothreitol (DTT) in PBS (25 mM), pH 7.4, for 1 hr at room temperature. After reduction, sample was eluted by incubating with pH 3.0 buffer (distilled water containing 20% acetonitrile and 1% formic acid) for 1 hr at room temperature. Purified ADC samples were then analyzed by mass spectrometry to quantify DAR or drug loading or kept frozen at −80° C. until further analysis.

Drug loading and percent maleimide ring opening of purified ADC samples were assessed using an Agilent 1290 Infinity II HPLC™ system coupled to an Agilent 6545 Quadrupole Time of Flight Mass Spectrometer™ (Agilent Technologies™, Santa Clara, CA). Extent of drug loading and percent maleimide ring opening at each time point was graphed using GraphPad Prism™ software (GraphPad Software™, San Diego, CA).

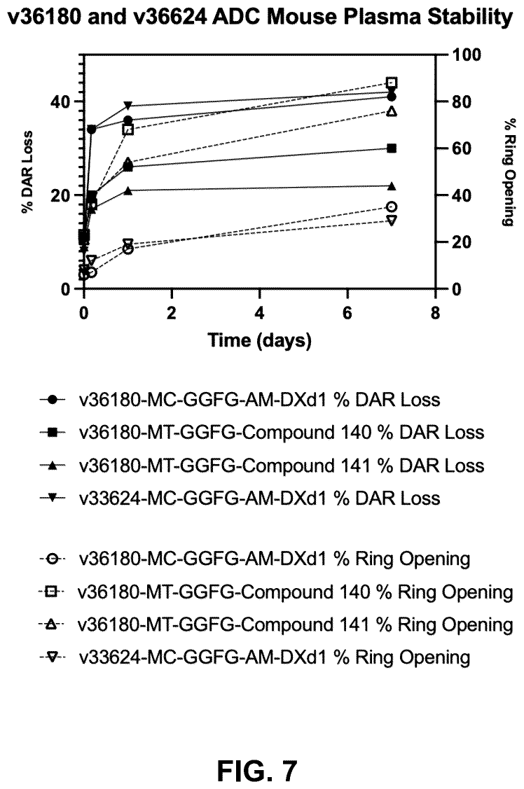

The results are shown in FIG. 7 and Table 20.1. Overall, the two DXd1 ADCs showed highly similar results for DAR loss over time and extent of maleimide ring opening. Both of these ADCs showed 41-42% DAR loss and 29-35% maleimide ring opening over 7 days, suggesting that the antibodies would not affect drug linker properties in this case. ADCs conjugated to Compound 140 or Compound 141 showed highly similar results for DAR loss over time and extent of maleimide ring opening that were different from the DXd1 ADCs. The Compound 140 and Compound 141 ADCs showed 22-30% DAR loss and 76-88% maleimide ring opening over 7 days. No significant linker drug decomposition was observed for any ADC tested.

TABLE 20.1
DAR Loss and Percent Maleimide Ring
Opening (Changes over 7 days)

| | Percent | Percent Maleimide |
|---|---|---|
| ADC | DAR Loss | Ring Opening |
| v36180-MC-GGFG-AM-DXd1 | 41 | 35 |
| v36180-MT-GGFG-Compound 140 | 30 | 88 |
| v36180-MT-GGFG-Compound 141 | 22 | 76 |
| v33624-MC-GGFG-AM-DXd1 | 42 | 29 |

### Example 21: Pharmacokinetic Study in Tg32 Mice

The pharmacokinetics of the humanized antibodies v36180, v37574, and three ADCs were assessed in humanized FcRn Tg32 mice as described below. This mouse model can be predictive of the pharmacokinetics of a drug in humans (see Avery et al. (2016) Utility of a human FcRn transgenic mouse model in drug discovery for early assessment and prediction of human pharmacokinetics of monoclonal antibodies, mAbs, 8:6, 1064-1078). The ADCs assessed were: M3-H₁L1 v36180-MC-GGFG-Compound 141 and M3-H18L6 v37574-MC-GGFG-Compound 141, and Codrituzumab (v37575-MC-GGFG-AM-DXd1). All ADCs were DAR8.

All test articles were administered at 5 mg/kg to hFcRn Tg32 mice (The Jackson Laboratory, Sacramento, CA; Stock #014565) by intravenous injection. For each test article, blood was collected from n=4 animals by retro-orbital bleed at 1, 3, and 6 hours and 1, 3, 7, 10, 14, 21 days post-dose. Blood was processed to serum and stored frozen at −80° C. in 96-well storage plates prior to pharmacokinetic analysis.

Test article concentrations were measured in mouse serum by sandwich ELISA utilizing an anti-human IgG1 Fc capture antibody (Jackson Immuno Research Labs, West Grove, PA; Cat. 709-005-098) and an HRP-conjugated anti-IgG1 Fab detection antibody (Jackson Immuno Research Labs; Cat. 109-035-097) for total IgG levels. Absorbance at 450 nm was measured using a Synergy™ H1 Hybrid Multi-Mode Plate Reader (BioTek Instruments, Winooski, VT). Sample data were analyzed using SoftMax® Pro 7.1 (Molecular Devices, San Jose, CA). Pharmacokinetics parameters were calculated from non-compartmental analysis using Phoenix WinNonlin™ software (Certara, Princeton, NJ).

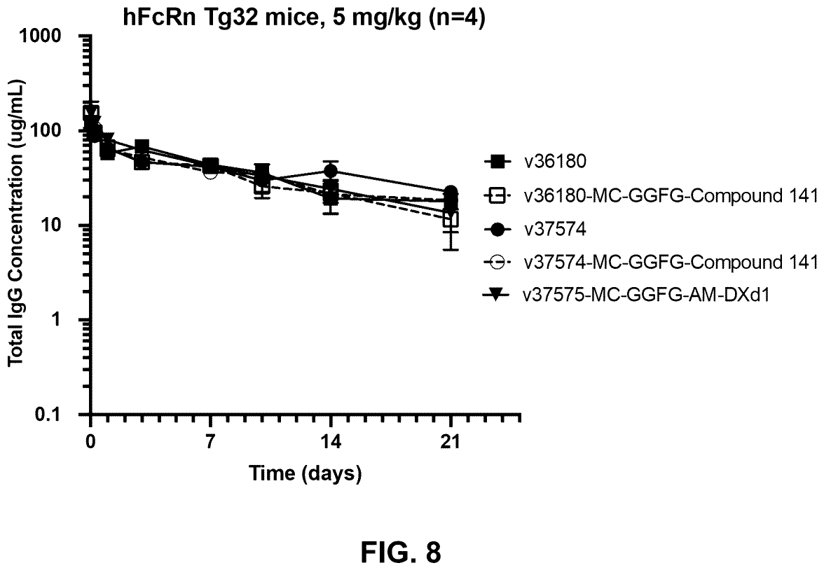

The results are shown in FIG. 8. Analysis in hFcRn Tg32 mice demonstrated the total IgG PK profiles of the ADCs were comparable to their naked antibody counterparts and to v37575, with typical antibody-like prolonged exposures. Elimination half-life of each antibody was determined and is shown in Table 21.1.

TABLE 21.1
Elimination Half-life of Antibodies and ADCs

| | Dose | Total IgG Half-life |
|---|---|---|
| Test Article | (mg/kg) | (Days) |
| v36180 | 5 | 9.7 |
| v36180-MC-GGFG-Compound 141 | 5 | 8.3 |
| v37574 | 5 | 15.4 |
| v37574-MC-GGFG-Compound 141 | 5 | 10.9 |
| v37575-MC-GGFG-AM-DXd1 | 5 | 8.5 |

Elimination half-life of the v36180 antibody was determined to be 9.7 days and elimination of the ADC v36180-MC-GGFG-Compound 141 was determined to be 8.3 days. Elimination half-life of the v37574 antibody was determined to be 15.4 days and elimination of the ADC v37574-MC-GGFG-Compound 141 was determined to be 10.9 days. The elimination half-life of v37575 was determined to be 8.5 days. All elimination half-lives were determined by non-compartmental analysis.

### Example 22: In Vivo Efficacy of M3 H₁L1 (v36180) ADCs in JHH-7 and NCI-H446 Cell Line-Derived Xenografts (Cdx)

M3 H₁L1 (v36180) ADCs were tested in JHH-7 and NCI-H446 CDX models to determine their in vivo efficacy and their relative anti-tumor activity compared to reference GPC3-targeting antibody BMS-986182 (v33624) conjugated to DXd1. An ADC of palivizumab (v21995) conjugated to DXd1 was included as a non-targeting control. This experiment was also designed to assess the activity of the M3 H₁L1 paratope compared to the reference antibody BMS-986182.

The studies were carried out as follows. Cancer cells (Table 22.1) suspended in a 1:1 mixture of PBS and Matrigel® were injected subcutaneously into the right front flank region of 6-7 week old female BALB/c nude mice. Tumors were measured using a caliper and tumor volume (V, mm³) was determined by the formula V=(L×W×W)/2, where L and W is the length and width of the tumor, respectively. When tumors reached a mean volume of ˜140 mm³, mice were randomized into treatment groups (JHH-7: n=7 per group; NCI-H₄₄₆: n=6 per group) and injected with a single intravenous dose of test article on day 0 (Table 22.2). Tumor volumes and body weights were monitored twice a week over a 28-day study period. Whole blood was collected retro-orbitally at multiple time points and processed to serum for pharmacokinetic (PK) analysis (as described in Example 23). All JHH-7 tumor-bearing mice were given dietary gel supplements from Day 4 to the end of study.

TABLE 22.1
Characteristics of CDX models

| | | | Inoculum and Volume | Mean Tumor |
|---|---|---|---|---|
| Xenograft | Cancer | GPC3 | of PBS mixed with | Volume at |
| Model | Type | Expression | Matrigel ® (1:1) | Day 0 (mm³) |
| JHH-7 | Liver | High | 1 × 10⁷ in 0.1 mL | ~135 |
| NCI-H446 | Lung | Mid | 5 × 10⁶ in 0.1 mL | ~138 |

TABLE 22.2
Treatment groups in each CDX model

| | | Dose |
|---|---|---|
| | | Level |
| Group | Test Article | (mg/kg) |
| 1 | Vehicle, 10 mM Na acetate, | 0 |
| | 9% sucrose, pH 4.5 (A5Su) | |
| 2 | v33624-MC-GGFG-AM-DXd1 DAR8 | 3 |
| 3 | v33624-MC-GGFG-AM-DXd1 DAR8 | 10 |
| 4 | v36180-MC-GGFG-AM-DXd1 DAR8 | 3 |
| 5 | v36180-MC-GGFG-AM-DXd1 DAR8 | 10 |
| 6 | v36180-MT-GGFG-Compound 140 DAR8 | 3 |
| 7 | v36180-MT-GGFG-Compound 141 DAR8 | 3 |
| 8 | v21995-MC-GGFG-AM-DXd1 DAR8 | 10 |

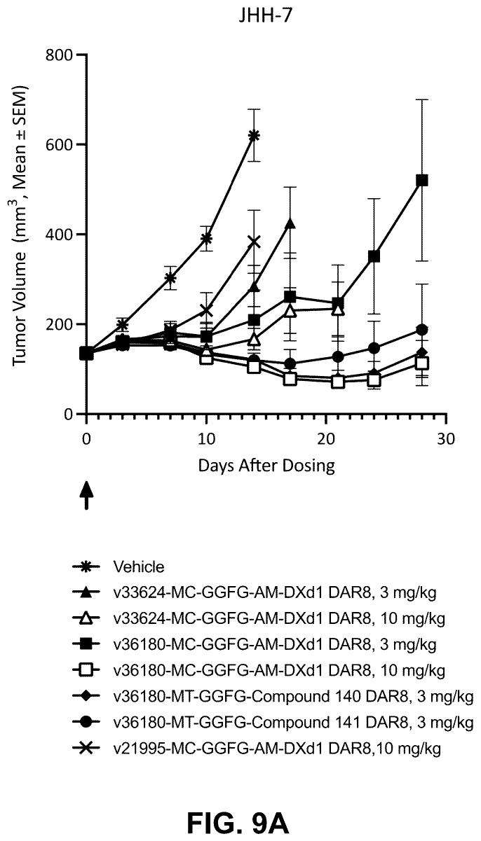

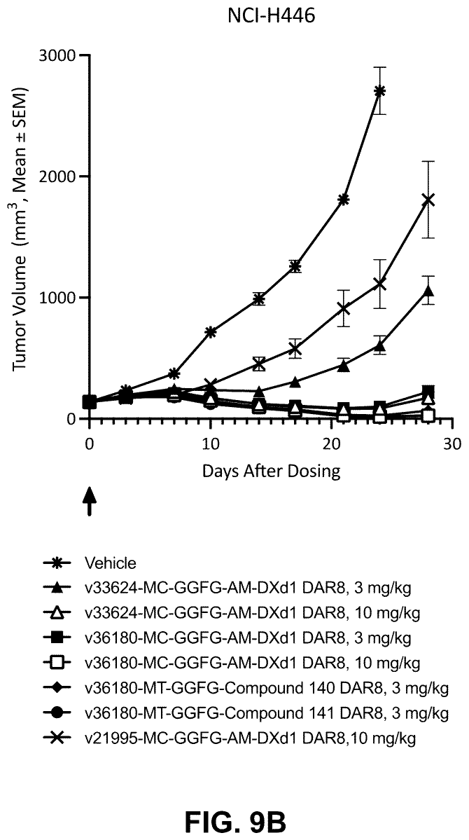

The results of the JHH-7 study are shown in FIG. 9A. The mean tumor volume plot for each group was terminated when >20% of mice were lost (e.g. humane endpoints reached due to body weight loss or tumors exceeding 2000 mm³). All anti-GPC3 ADCs exhibited varying degrees of anti-tumor activity as measured by inhibition of tumor growth and displayed greater anti-tumor activity when compared to the non-targeting ADC (v21995-MC-GGFG-AM-DXd1 DAR8) at the same dose level. At 3 mg/kg, v36180-MT-GGFG-Compound 140 DAR8 and v36180-MT-GGFG-Compound 141 DAR8 demonstrated greater tumor growth inhibition compared to v36180-MC-GGFG-AM-DXd1 DAR8. Comparing ADCs with the same payload but conjugated to different antibodies, v36180-MC-GGFG-AM-DXd1 DAR8 exhibited greater anti-tumor activity over the reference antibody v33624-MC-GGFG-AM-DXd1 DAR8 at both dose levels (3 mg/kg and 10 mg/kg).

The results of the NCI-H446 study are shown in FIG. 9B. The mean tumor volume plot for each group was terminated when >20% of mice were lost (e.g. humane endpoints reached due to body weight loss or tumors exceeding 2000 mm³). At 3 mg/kg, all anti-GPC3 ADCs showed anti-tumor activity and tumor regression was observed at study termination in most mice receiving v36180-MT-GGFG-Compound 140 or v36180-MT-GGFG-Compound 141 DAR8. Antibody drug conjugate v36180-MC-GGFG-AM-DXd1 DAR8 demonstrated superior activity over v33624-MC-GGFG-AM-DXd1 DAR8 at the 3 mg/kg dose. At 10 mg/kg, v36180-MC-GGFG-AM-DXd1 DAR8 and v33624-MC-GGFG-AM-DXd1 DAR8 strongly inhibited tumor growth compared to the non-targeting ADC (v21995-MC-GGFG-AM-DXd1 DAR8).

### Example 23: Pharmacokinetics of ADCs in In Vivo Efficacy Models

Serum was collected from the xenograft studies described in Example 22, as noted, and analyzed for the pharmacokinetics (PK) of the ADCs as described below. An ADC of palivizumab (v21995) conjugated to DXd1 was used as a non-targeted control.

Test article concentrations were measured in mouse serum by sandwich ELISA utilizing an anti-human IgG1 Fc capture antibody (Jackson Immuno Research Labs, West Grove, PA; Cat. 709-005-098) and an HRP-conjugated anti-IgG1 Fab detection antibody (Jackson Immuno Research Labs; Cat. 109-035-097) for total IgG levels. Absorbance at 450 nm was measured using a Synergy™ H1 Hybrid Multi-Mode Plate Reader (BioTek Instruments, Winooski, VT). Sample data were analyzed using SoftMax® Pro 7.1 (Molecular Devices, San Jose, CA). Pharmacokinetics parameters were calculated from non-compartmental analysis using Phoenix WinNonlin™ software (Certara, Princeton, NJ).

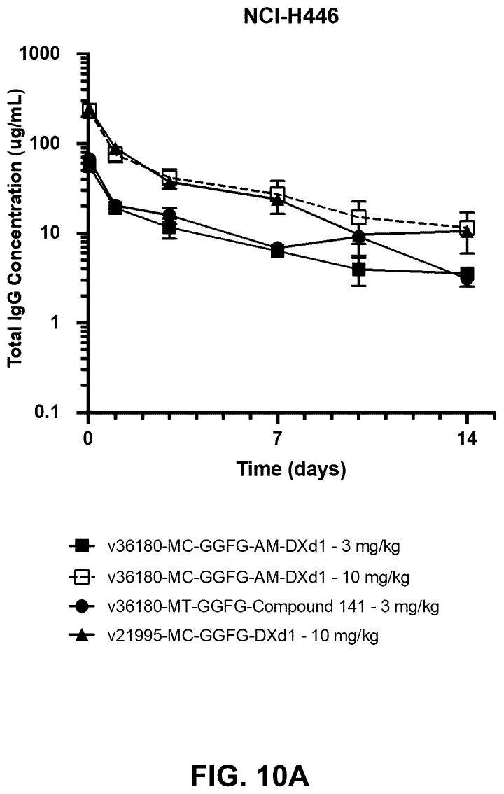

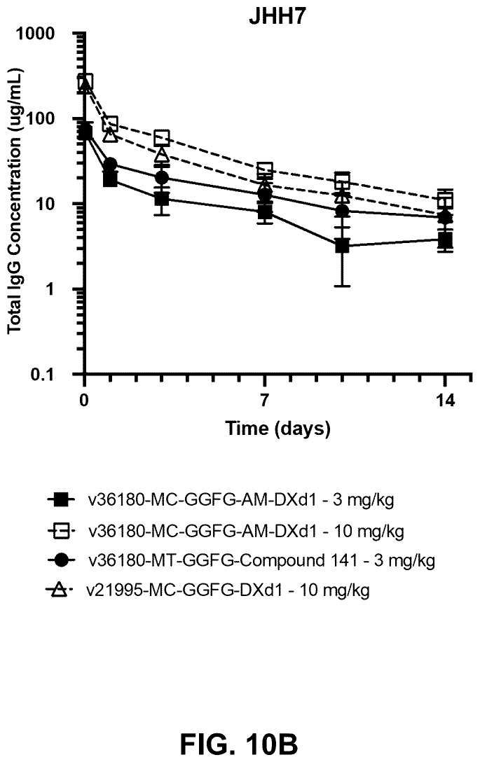

In both tumor models: NCI-H446 and JHH-7, the total IgG concentrations of all ADCs demonstrated PK profiles with typical antibody-like prolonged exposures. Clear dose-dependency for the ADC v36180-MC-GGFG-AM-DXd1 was observed across the 2 different dosing concentrations in both models. In the NCI-H446 model (FIG. 10A), Cmax for doses 3 mg/kg and 10 mg/kg were approximately 56 μg/mL and 234 μg/mL, respectively. In the JHH7 model (FIG. 10B), Cmax for doses 3 mg/kg and 10 mg/kg were approximately 67 μg/mL and 271 μg/mL, respectively.

Table 23.1 provides a summary of Cmax and elimination half-lives determined in both models. All elimination half-lives were determined by non-compartmental analysis.

TABLE 23.1
Cmax and Elimination Half-life of Antibodies and ADCs

| Xenograft | | Dose | Cmax | Total IgG Half- |
|---|---|---|---|---|
| Model | Test Article | (mg/kg) | (μg/ml) | life (Days) |
| NCI-H446 | v36180-MC-GGFG- | 3 | 56.1 | 5.3 |
| | AM-DXd1 | | | |
| | v36180-MC-GGFG- | 10 | 233.6 | 5.7 |
| | AM-DXd1 | | | |
| | v36180-MT-GGFG- | 3 | 67.2 | 5.2 |
| | Compound 141 | | | |
| | v21995-MC-GGFG- | 10 | 247.4 | 4.2 |
| | DXd1 | | | |
| JHH7 | v36180-MC-GGFG- | 3 | 66.9 | 5.3 |
| | AM-DXd1 | | | |
| | v36180-MC-GGFG- | 10 | 271.5 | 6.0 |
| | AM-DXd1 | | | |
| | v36180-MT-GGFG- | 3 | 74.1 | 6.1 |
| | Compound 141 | | | |
| | v21995-MC-GGFG- | 10 | 239.6 | 6.0 |
| | DXd1 | | | |

In the NCI-H446 model, the elimination half-life of the ADC v36180-MC-GGFG-AM-DXd1 was determined to be 5.3 days at the 3 mg/kg dose and 5.7 days at the 10 mg/kg dose. The elimination half-life of the ADC v36180-MT-GGFG-Compound 141 was determined to be 5.7 days. The half-life of the non-targeting control v21995 ADC was 4.2 days. In the JHH7 model, the elimination half-life of the ADC v36180-MC-GGFG-AM-DXd1 was determined to be 5.3 days at the 3 mg/kg dose and 6 days at the 10 mg/kg dose. The elimination half-life of the ADC v36180-MT-GGFG-Compound 141 was determined to be 6.1 days.

### Example 24: In Vivo Efficacy of M3 H₁L1 (V36180) ADC and M3 H18L6 (V37574) ADC in JHH-7 and NCI-H446 CDX Models

The in vivo efficacy of ADCs comprised of v36180 and v37574 humanized paratopes were compared in JHH-7 and NCI-H446 CDX models. An ADC of palivizumab (v21995) conjugated to compound 141 was included as a non-targeting control.

The studies were carried out as follows. Cancer cells suspended in a 1:1 mixture of PBS and Matrigel® were injected subcutaneously into the right front flank region of 8-10 week old female BALB/c nude mice as summarized in Table 24.1. When tumors reached a mean volume of 140-155 mm³, mice were randomized into treatment groups (JHH-7: n=8 per group; NCI-H₄₄₆: n=6 per group) and injected intravenously with a single dose of test article as identified in Table 24.2. Tumor volumes and body weights were monitored twice weekly over a 28-day study period as described in Example 22. Whole blood was collected retro-orbitally at multiple time points and processed to serum for future PK analysis. Dietary gel supplements were provided to all JHH-7 study mice from day 0 to end of study.

TABLE 24.1
Characteristics of CDX models

| | | | Inoculum and Volume | Mean Tumor |
|---|---|---|---|---|
| Xenograft | Cancer | GPC3 | of PBS mixed with | Volume at |
| Model | Type | Expression | Matrigel ® (1:1) | Day 0 (mm³) |
| JHH-7 | Liver | High | 1 × 10⁷ in 0.1 mL | ~144 |
| NCI-H446 | Lung | Mid | 5 × 10⁶ in 0.1 mL | ~150 |

TABLE 24.2
Treatment groups in each CDX model

| | | Dose |
|---|---|---|
| | | Level |
| Group | Test Article | (mg/kg) |
| 1 | Vehicle, 10 mM Na acetate, | 0 |
| | 9% sucrose, pH 4.5 (A5Su) | |
| 2 | v36180-MC-GGFG-Compound 141 DAR8 | 0.3 |
| 3 | v36180-MC-GGFG-Compound 141 DAR8 | 1 |
| 4 | v36180-MC-GGFG-Compound 141 DAR8 | 3 |
| 5 | v37574-MC-GGFG-Compound 141 DAR8 | 0.3 |
| 6 | v37574-MC-GGFG-Compound 141 DAR8 | 1 |
| 7 | v37574-MC-GGFG-Compound 141 DAR8 | 3 |
| 8 | v21995-MC-GGFG-Compound 141 DAR8 | 3 |

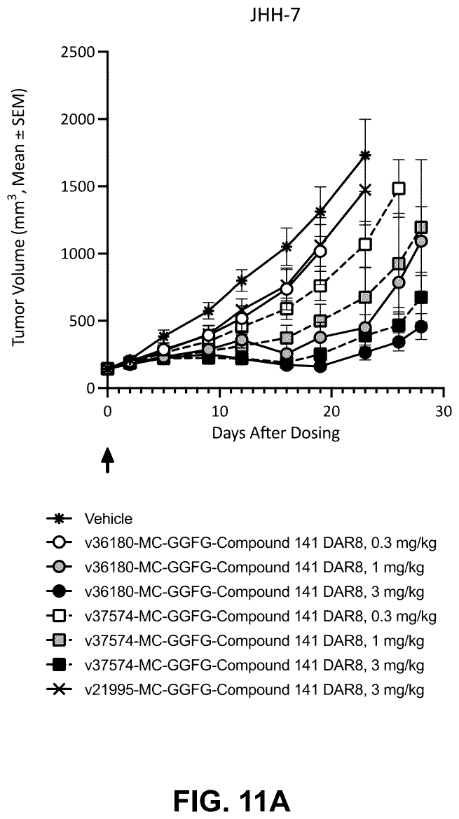

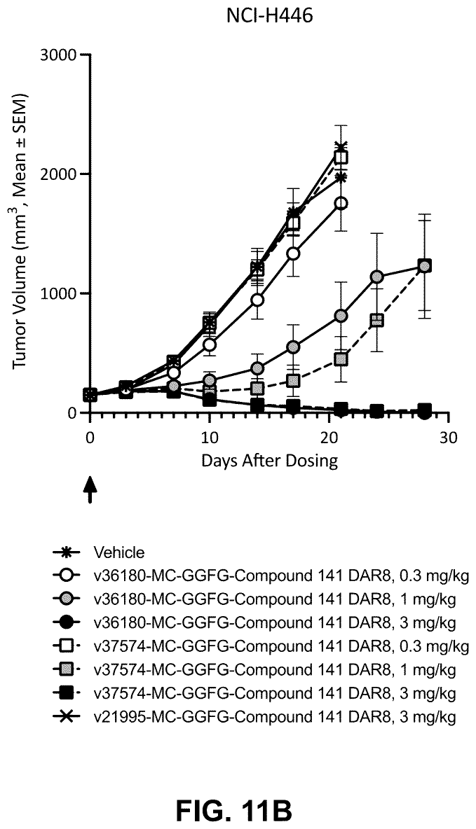

Study results for JHH-7 are shown in FIG. 11A. Cachexia, unrelated to treatment, was frequently observed and was the main reason for euthanasia before the end of the study period. As such, the mean tumor volume plot for each group was terminated only when >20% of mice were euthanized due to tumor volumes exceeding 2000 mm³ or when <4 mice remained on study. The ADCs v36180-MC-GGFG-Compound 141 DAR8 and v37574-MC-GGFG-Compound 141 DAR8 inhibited tumor growth at 1 mg/kg and 3 mg/kg but had negligible anti-tumor activity at 0.3 m/kg. The non-targeting ADC (v21995-MC-GGFG-Compound 141 DAR8) displayed minimal activity at 3 mg/kg. Both v36180-MC-GGFG-Compound 141 DAR8 and v37574-MC-GGFG-Compound 141 DAR8 showed comparable anti-tumor activity within each of the 3 dose levels.

The results for the NCI-H446 study are shown in FIG. 11B. The mean tumor volume plot for each group was terminated when >20% of mice were lost (e.g. humane endpoints reached due to body weight loss or tumors exceeding 2000 mm³). The ADCs v36180-MC-GGFG-Compound 141 DAR8 and v37574-MC-GGFG-Compound 141 DAR8 exhibited comparable anti-tumor activity across the various dose levels. Both ADCs displayed negligible anti-tumor activity at 0.3 mg/kg but inhibited tumor growth at 1 mg/kg. Tumor regression was observed in most mice treated with 3 mg/kg of v36180-MC-GGFG-Compound 141 DAR8 and 3 mg/kg of v37574-MC-GGFG-Compound 141. The non-targeting ADC (v21995-MC-GGFG-Compound 141 DAR8) showed minimal activity at 3 mg/kg.

In both models, both humanized variants of the M3 paratope showed similar efficacy with the same payload.

### Example 25: In Vivo Efficacy of M3 H18L6 (v37574) ADCs in Liver Cancer CDX Models

The anti-tumor activity of M3 H18L6 ADCs was further tested in a larger panel of liver cancer CDX models.

The studies were carried out as follows. Cancer cells suspended in a 1:1 mixture of PBS and Matrigel® were injected subcutaneously into the right front flank region of 8-10 week old female BALB/c nude mice as described in Table 25.1. When tumors reached a mean volume of 140-160 mm³, mice were randomized into treatment groups and injected intravenously with a single dose of test article at day 0 as shown in Table 25.2. Tumor volumes and body weights were monitored twice weekly over a 28-day study period as described in Example 22. Whole blood was collected retro-orbitally at multiple time points and processed to serum for future PK analysis. Dietary gel supplements were provided to all HepG2, Hep3B, and PLC/PRF/5 study mice from day 4, 15, and 14, respectively, to the end of study.

TABLE 25.1
Characteristics of CDX Models

| | | | Inoculum and | Mean Tumor |
|---|---|---|---|---|
| | | | Volume of PBS | Volume at |
| Xenograft | Cancer | GPC3 | mixed with | Day 0 |
| Model | Type | Expression | Matrigel ® (1:1) | (mm³) |
| HepG2 | Liver | High | 1 × 10⁷ in 0.1 mL | ~150 |
| Hep3B | Liver | High | 5 × 10⁶ in 0.1 mL | ~150 |
| Huh-7 | Liver | Mid | 1 × 10⁷ in 0.2 mL | ~150 |
| PLC/PRF/5 | Liver | Mid | 1 × 10⁷ in 0.2 mL | ~155 |

TABLE 25.2
Treatment groups in each CDX model

| | | Dose | |
|---|---|---|---|
| | | Level | |
| Group | Test Article | (mg/kg) | n |
| 1 | Vehicle, 10 mM Na acetate, 9% sucrose, pH 4.5 | 0 | 6 |
| 2 | v37574-MC-GGFG-AM-Compound 139 DAR8 | 8 | 6 |
| 3 | v37574-MC-GGFG-Compound 141 DAR8 | 8 | 6 |
| 4 | v37574-MC-GGFG-AM-Compound 139 DAR4 | 8 | 6 |
| 5 | v37574-MC-GGFG-Compound 141 DAR4 | 8 | 6 |
| 6 | v21995-MC-GGFG-AM-Compound 139 DAR8 | 8 | 5 |
| 7 | v21995-MC-GGFG-Compound 141 DAR8 | 8 | 5 |

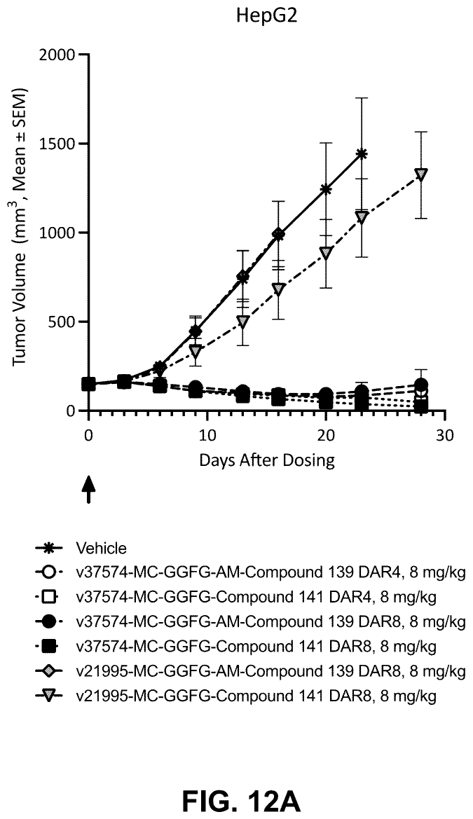

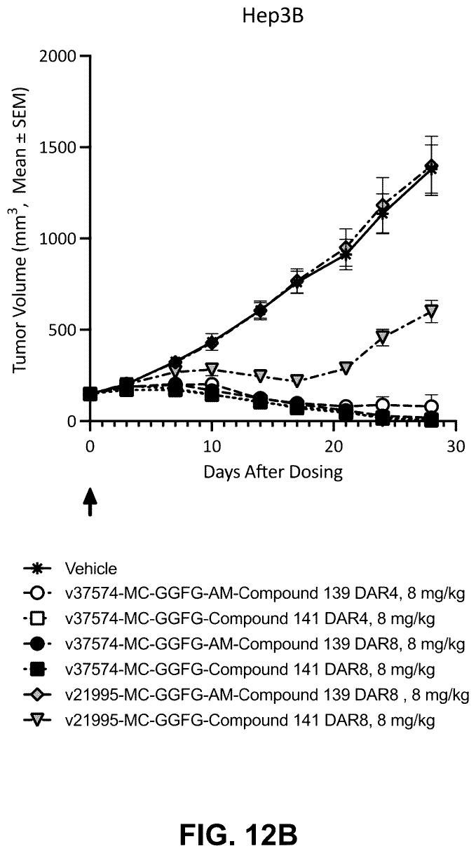

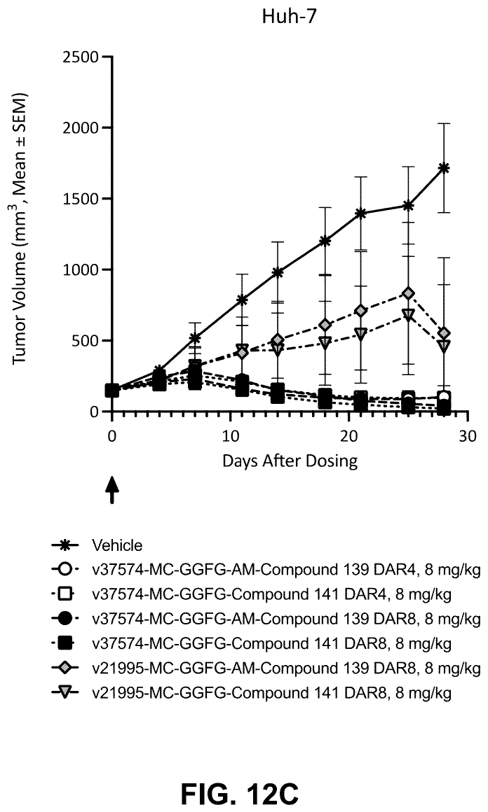

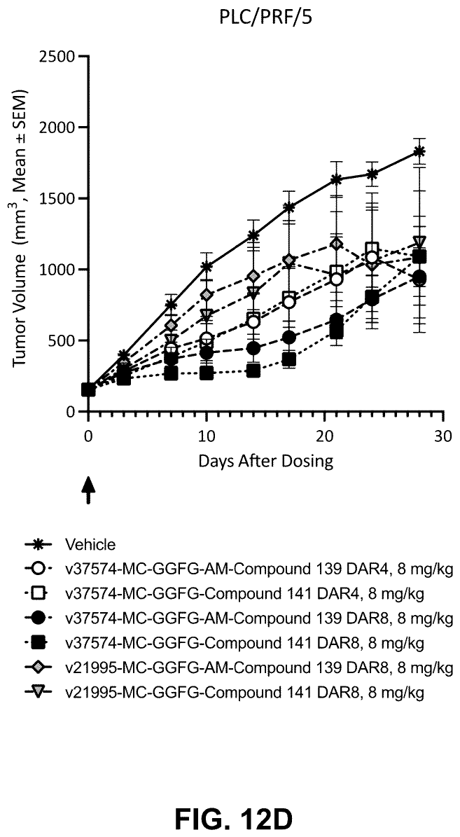

Results for the HepG2 model are shown in FIG. 12A. The mean tumor volume plot for each group was terminated when >20% of mice were lost (e.g. humane endpoints reached due to body weight loss or tumors exceeding 2000 mm³). All v37574 ADCs demonstrated strong anti-tumor activity relative to the non-targeting v21995 ADCs. Tumor regression was frequently observed in mice treated with v37574 ADCs. The anti-tumor activity of 37574 ADCs did not substantially differentiate by DAR or payload.

Results for Hep3B are shown in FIG. 12B. All v37574 ADCs demonstrated strong anti-tumor activity. Tumor regression was frequently observed. The anti-tumor activity of 37574 ADCs did not differentiate by DAR or payload. Non-targeting ADC v21995-MC-GGFG-AM-Compound 139 was comparable to the vehicle control but v21995-MC-GGFG-Compound 141 modestly inhibited tumor growth.

Results for the Huh-7 model are shown in FIG. 12C. All v37574 ADCs demonstrated strong anti-tumor activity, with minimal differences between DAR4 and DAR8, and AM-Compound 139 and Compound 141. Tumor regression was frequently observed in mice treated with v37574 ADCs. Non-targeting v21995 ADCs delayed tumor growth but to a much lesser extent compared to v37574 ADCs.

Results for the PLC/PRF/5 model are shown in FIG. 12D. All v37574 ADCs delayed tumor growth. DAR 8 ADCs exhibited greater activity compared to DAR4 ADCs, with minimal differences observed between payloads. Non-targeting v21995 ADCs modestly delayed tumor growth but to a lesser extent compared to v37574 DAR8 ADCs. This difference was most pronounced at 2-3 weeks after dosing and was gradually reduced over time.

Overall, v37574 ADCs demonstrated in vivo efficacy in multiple liver cancer CDX models.

### Example 26: In Vivo Efficacy of H18L6 (v37574) ADCs in Two Patient-Derived Xenograft (PDX) Models of Hepatocellular Carcinoma (HCC)

The anti-tumor activity of H18L6 (v37574) ADCs was investigated in two PDX models of hepatocellular carcinoma.

The studies were carried out as follows. Tumor fragments (approximately 2 to 3 mm³) from stock mice bearing LI1025 and LI1037 patient-derived xenografts (HuPrime® Liver Cancer Xenograft Models, Crown Bioscience Inc.) were implanted subcutaneously into 6-8 week old female BALB/c nude mice as described in Table 26.1. IHC results of historic tumor samples from non-study mice show that LI1025 and LI1037 express mid and high levels of GPC3, respectively. When tumors reached a mean volume of 140-170 mm³, mice were randomized into treatment groups and injected intravenously with a single dose of test article at day 0 as shown in Table 26.2. Tumor volumes and body weights were monitored twice weekly over a 28-day study period as described in Example 22. Whole blood was collected retro-orbitally at multiple timepoints and processed to serum for future PK analysis.

TABLE 26.1
Characteristics of PDX Models

| | | | | Mean Tumor |
|---|---|---|---|---|
| | | GPC3 | Age of Study | Volume at |
| Model | Cancer Type | Expression | Mice (week) | Day 0 (mm³) |
| LI1025 | Liver/HCC | Mid | 7-8 | ~147 |
| LI1037 | Liver/HCC | High | 6-7 | ~164 |

TABLE 26.2
Treatment groups in each PDX model

| | | Dose | |
|---|---|---|---|
| | | Level | |
| Group | Test Article | (mg/kg) | n |
| 1 | Vehicle, 10 mM Na acetate, 9% sucrose, pH 4.5 | 0 | 3 |
| 2 | v37574-MC-GGFG-AM-Compound 139 DAR4 | 8 | 3 |
| 3 | v37574-MC-GGFG-AM-Compound 139 DAR8 | 8 | 3 |
| 4 | v21995-MC-GGFG-AM-Compound 139 DAR8 | 8 | 3 |

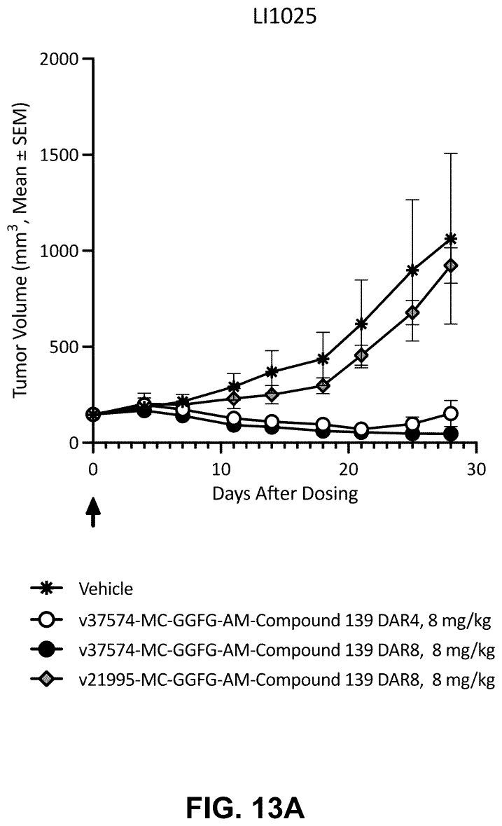

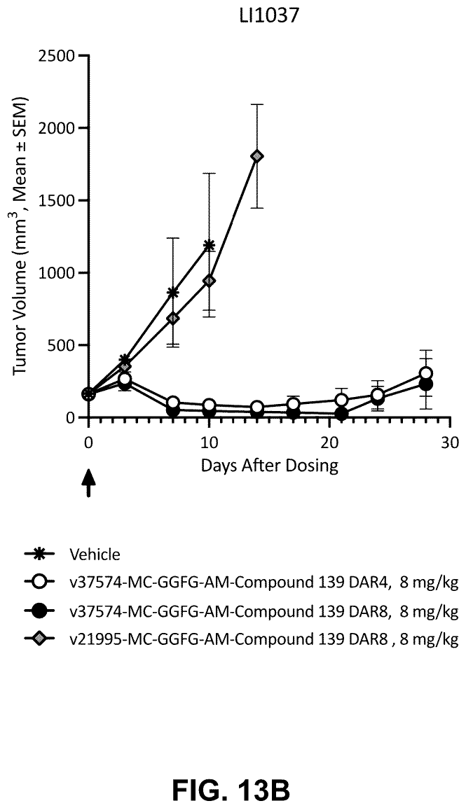

Results for LI1025 are shown in FIG. 13A. DAR4 and DAR8 v37574 ADCs showed strong anti-tumor activity. At the end of the study period (day 28), tumors were partially regressed in 1 of 3 mice and 3 of 3 mice treated with DAR4 and DAR8 v37574 ADCs, respectively. No substantial anti-tumor activity was observed with the non-targeting control (v21995) ADC.

Results for LI1037 are shown in FIG. 13B. The mean tumor volume plot for each group was terminated when <3 mice remained on study. DAR4 and DAR8 v37574 ADCs showed strong anti-tumor activity. At day 28, 1 of 3 mice treated with DAR4 v37574 ADC showed complete tumor regression, and 2 of 3 mice treated with DAR8 v37574 ADC showed partial tumor regression. No substantial anti-tumor activity was observed with the non-targeting control (v21995) ADC.

Overall, v37574 ADCs demonstrated in vivo efficacy in GPC3-expressing PDX models of hepatocellular carcinoma.

### Example 27: Bystander Activity of Anti-GPC3 Antibody-Drug Conjugates

The ability of ADCs of humanized variant M3-H18L6 (v37574) to exert a bystander killing effect on cancer cells was assessed as described below. Bystander killing can occur after target-specific uptake of an ADC into an antigen-positive cell. In this case, catabolism of the ADC results in release of payload or an active catabolite, which then crosses the cell membrane of nearby cells to elicit death.

The M3-H18L6 ADCs tested were v37574-MC-GGFG-AM-Compound 139 at DAR8 and DAR4 as well as v37574-MC-GGFG-AM-DXd1 at DAR8. The MC-GGFG-AM-DXd1 drug linker is known to have bystander activity. An ADC of reference antibody codrituzumab v37575-MC-GGFG-AM-DXd1 at DAR8 was also tested. Negative (non-GPC3-targeting) controls palivizumab v21995-MC-GGFG-AM-DXd1 and palivizumab v21995-MC-GGFG-AM-Compound 139, were also assessed.

GPC3-high HepG2 (hepatocellular carcinoma) or GPC3-mid JHH-5 (hepatocellular carcinoma) cells were seeded either as mono-cultures or as co-cultures with GPC3-negative SNU-601 (gastric carcinoma) cells. This was done by seeding 25,000 HepG2 or 15,000 JHH-5 cells with 5,000 SNU-601 cells in each well of a 48-well plate in 100 μL assay media (RPMI-1640+10% FBS). ADCs were diluted in assay media and added to the cell-containing plates to a final concentration of 1 nM. Cells were incubated with test ADCs for 4 d at 37° C./5% CO₂ and detached by Cell Dissociation Buffer (Invitrogen). Cells were stained using a viability dye, YO—PRO®-1 (ThermoFisher Scientific, Waltham, MA), and v33624 conjugated to AF647 (previously described in Example 12). After 20 min incubation at room temperature, cells were washed in FACS buffer (PBS+1% FBS) and analyzed on the BD Fortessa™ flow cytometer (BD Biosciences, San Jose, CA). Dead cells were gated out by YO—PRO®-1 staining. The number of HepG2/JHH-5 and SNU-601 cells was then determined by the number of events in the GPC3-positive and GPC3-negative gates, respectively. % SNU-601 viability was calculated as the number of SNU-601 cells in treated conditions divided by the number of SNU-601 cells in untreated conditions.

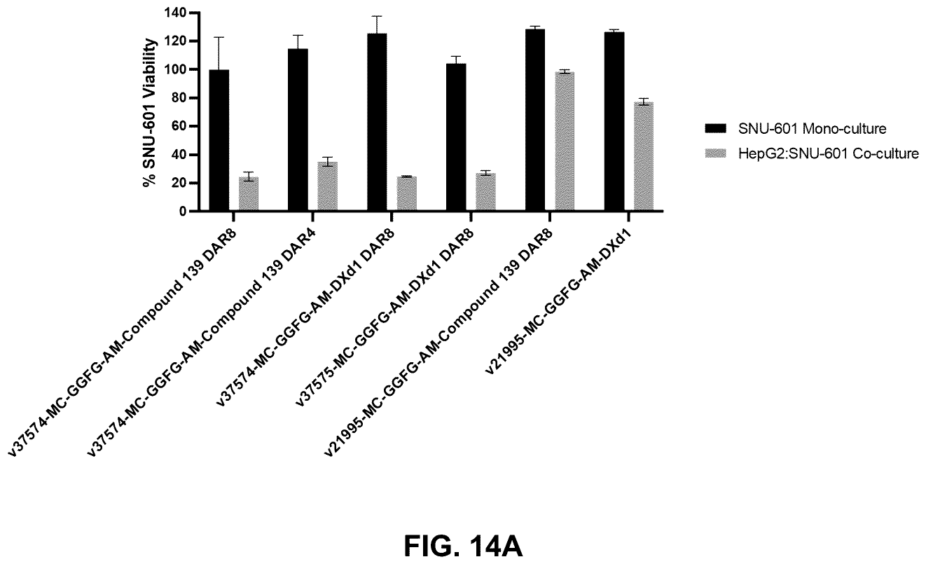

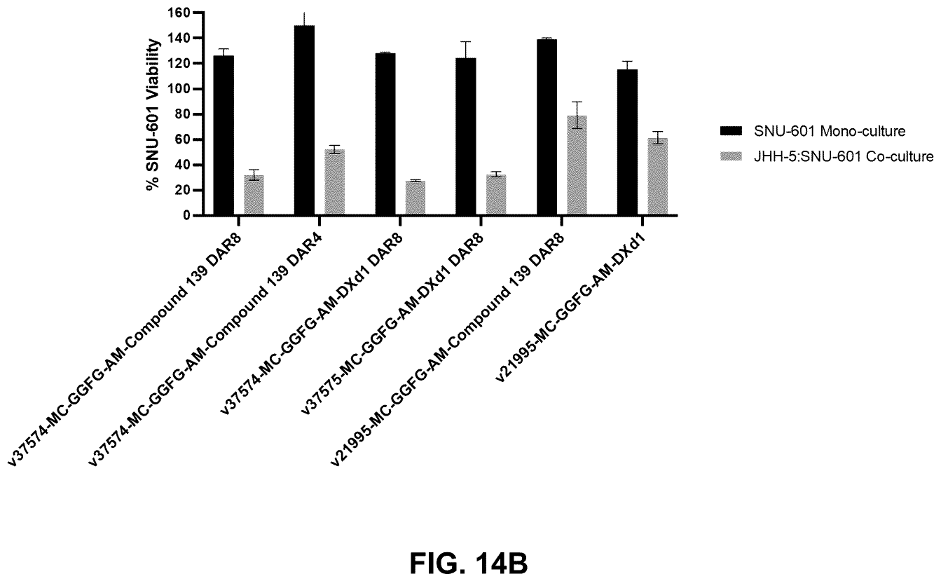

The results are shown in FIG. 14A and FIG. 14B. Bystander effect was evaluated by comparing the viability of GPC3-negative SNU-601 cells treated as a mono-culture (black bars) with that of the cells treated as a co-culture with GPC3-positive HepG2 or JHH-5 cells (grey bars). A greater decrease in viability in co-culture compared with mono-culture indicated a higher bystander effect. In GPC3-negative SNU-601 mono-cultures, neither v37574 ADCs nor v37575 ADCs demonstrated a decrease in viability compared to non-targeting ADCs, as expected. In contrast, ADCs of v37574 and v37575 exhibited a greater reduction in SNU-601 viability compared to non-targeting ADCs in co-cultures with GPC3-high HepG2 (see FIG. 14A) or GPC3-mid JHH-5 cells (see FIG. 14B), indicating a bystander effect. MC-GGFG-AM-DXd1 conjugates of v37574 and v37575 demonstrated comparable bystander activity in the cell lines tested. v37574-MC-GGFG-AM-Compound 139 conjugate also exhibited comparable bystander activity to v37574-MC-GGFG-AM-DXd1 in the cell lines tested.

### Example 28: Assessment of Specificity of Anti-GPC3 Antibody

Membrane Proteome Array™ (Integral Molecular, Philadelphia, PA, USA) was used to screen for specific off-target binding interactions for antibody v38592. This anti-GPC3 antibody has amino acid sequences that are identical to v37574, except that the cDNA encoding the heavy chains of v38592 included a C-terminal lysine. The majority of the C-terminal lysine is cleaved once the antibody is secreted from the cell it is produced in.

Briefly, the study consisted of three phases: phase (1) determination of assay screening conditions, phase (2) membrane proteome array (library) screen and phase (3) protein target validation. In phase (1), conditions appropriate for detecting v38592 binding by high-throughput flow cytometry were determined, including the optimal antibody concentration and cell type for screening (two cell types were tested, HEK293T and avian QT6). In phase (2), using optimal conditions determined in phase 1, v38592 was screened against the library of over 6000 human membrane proteins (individually expressed in unfixed HEK293T cells), including 94% of all single-pass, multi-pass, and GPI-anchored proteins, including GPCRs, ion channels and transporters. In phase (3), each protein target hit from the screen stage (potential off-target interactions) was assessed in titration experiment using flow cytometry.

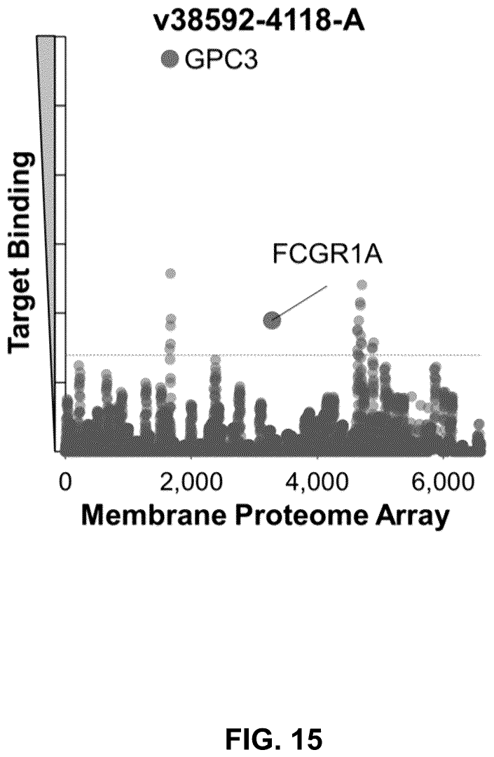

Phase (1) determined that HEK293T cells and an antibody concentration of 1.25 μg/mL were optimal for library screening. As shown in FIG. 15, library screening resulted in no validated protein target hits other than the primary target GPC3, and the target FcgR1A which binds to the Fc portion of the antibody. This data indicates that the humanized antibody v38592 was specific for the primary target, GPC3.

### Example 29: Functional Characterization of Anti-GPC3 ADCs—Binding to Human and Cynomolgus GPC3

The cross-reactivity of DAR 4 and DAR 8 MC-GGFG-AM-Compound 139 conjugates of humanized variant v38592 to human and cynomolgus monkey GPC3 was assessed by flow cytometry using transfected CHO—S cells as described below. Humanized variant v37574 (M3-H₁₈L6, lacking the C-terminal lysine residue in heavy chains) was used as a positive control, and v21995 was used as a negative control.

Briefly, CHO—S cells were transiently transfected for −24 hours with a pTT5-based expression plasmid encoding human GPC3 or cynomolgus monkey GPC3, 0.5 μg DNA per 1 million cells, using the Neon™ Transfection System (Thermo Fisher Scientific Corp., Waltham, MA), to transiently express human or cynomolgus monkey GPC3. Following transfection, cells were seeded at 50,000 cells/well in V-bottom 96-well plates and treated with antibody for 24 hours at 4° C. to prevent internalization. Following incubation, cells were washed and stained with anti-human IgG Fc AF647 conjugate (Jackson Immuno Research Labs, West Grove, PA; Cat. No. 109-605-098) at 4° C. for 30 min. Following incubation and washing, fluorescence was detected by flow cytometry on a BD LSRFortessa™ Cell Analyzer (BD Biosciences, Franklin Lake, NJ) with 1,000 minimum events collected per well. AF647/APC-A GeoMean (fluorescence signal geometric mean, proportional to anti-Human AF647 binding) in live cell population was plotted against antibody concentration using GraphPad Prism Version 9 (GraphPad Software, San Diego, CA). The Bmax and Kd values were derived from the “Specific binding with Hill slope” nonlinear regression curve fit in GraphPad Prism Version 9.

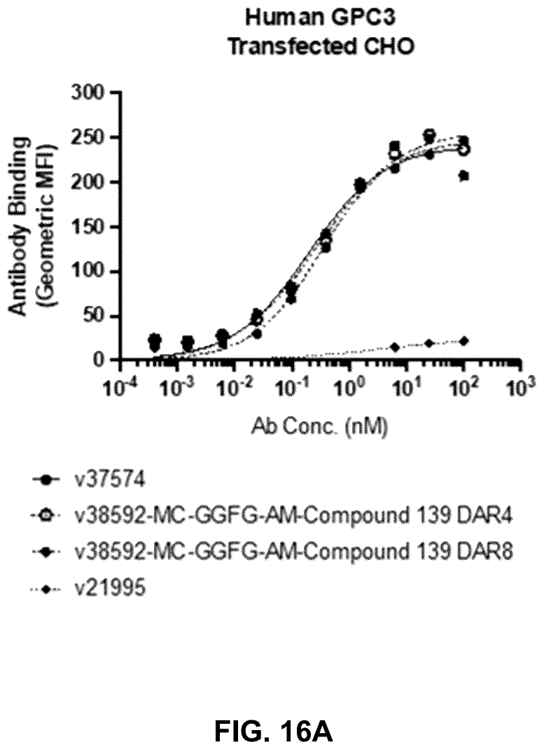

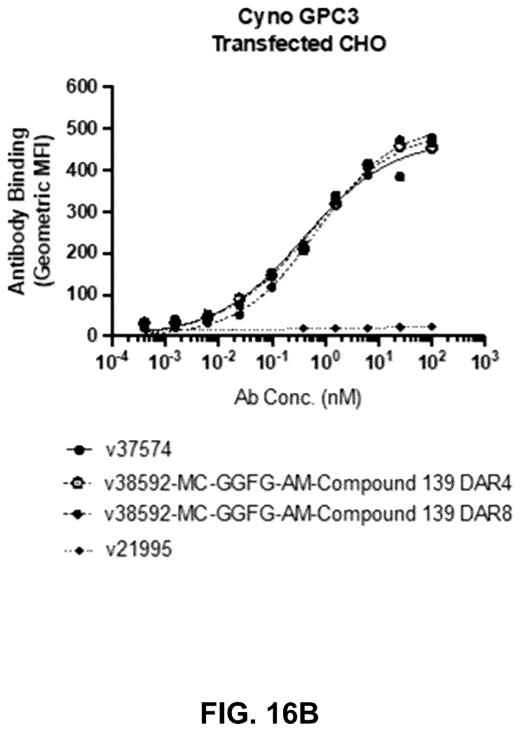

The results are shown in Table 29.1 and FIG. 16A and FIG. 16B. v37574 showed comparable binding to human and cynomolgus GPC3 on CHO—S transfected cells and v21995 exhibited minimal binding to CHO—S transfected cells as expected. ADCs v38592-MC-GGFG-AM-Compound 139 DAR 4 and DAR 8 demonstrated comparable binding affinity and maximal binding to v37574 in both human and cynomolgus GPC3 on CHO—S transfected cells. Both ADCs also demonstrated similar binding profiles between CHO—S cells expressing human GPC3 and cynomolgus monkey GPC3. These results show that minimal impact to cross-reactivity between human and cynomolgus monkey GPC3 was seen with MC-GGFG-AM-Compound 139 conjugation.

TABLE 29.1
Binding to Human and Cynomolgus Monkey GPC3

| | | | Apparent Kd |
|---|---|---|---|
| Antibody | Cells | Bmax | (nM) |
| v37574 | CHO-S | 242 | 0.20 |
| v38592-MC-GGFG-AM- | Human GPC3 | 259 | 0.29 |
| Compound 139, DAR 4 | | | |
| v38592-MC-GGFG-AM- | | 247 | 0.33 |
| Compound 139, DAR 8 | | | |
| v21995 | | I.C. | >100 |
| v37574 | CHO-S | 474 | 0.39 |
| v38592-MC-GGFG-AM- | Cyno GPC3 | 506 | 0.55 |
| Compound 139, DAR 4 | | | |
| v38592-MC-GGFG-AM- | | 511 | 0.70 |
| Compound 139, DAR 8 | | | |
| v21995 | | I.C. | >100 |

I.C.—Incomplete curves

### Example 30: Functional Characterization of Anti-GPC3 ADCs—Tumor Cell Binding

The on-cell binding capabilities of the DAR 4 and DAR 8 MC-GGFG-AM-Compound 139 conjugates of humanized variant v38592 to various GPC3-expressing tumor cells was assessed by flow cytometry as described below. Cell lines investigated included GPC3-hi HepG2 and JHH-7 cells, GPC3-mid JHH-5 cells, as well as GPC3-negative SNU-601 cells. Humanized variant v37574 was used as a positive control, and v21995 was used as a negative control.

Briefly, cells were seeded at 50,000 cells/well in V-bottom 96-well plates and treated with antibody for 24 hours at 4° C. to prevent internalization. Following incubation, cells were washed and stained with anti-Human IgG Fc AF647 conjugate (Jackson Immuno Research Labs, West Grove, PA; Cat. No. 109-605-098) at 4° C. for 30 min. Following incubation and washing, fluorescence was detected by flow cytometry on a BD LSRFortessa™ Cell Analyzer (BD Biosciences, Franklin Lake, NJ) with 1,000 minimum events collected per well. AF647/APC-A GeoMean (fluorescence signal geometric mean, proportional to anti-Human AF647 binding) in live cell population was plotted against antibody concentration using GraphPad Prism Version 9 (GraphPad Software, San Diego, CA). The Bmax, Curve Hill Slope (h) and Kd values were derived from the “Specific binding with Hill slope” nonlinear regression curve fit in GraphPad Prism Version 9.

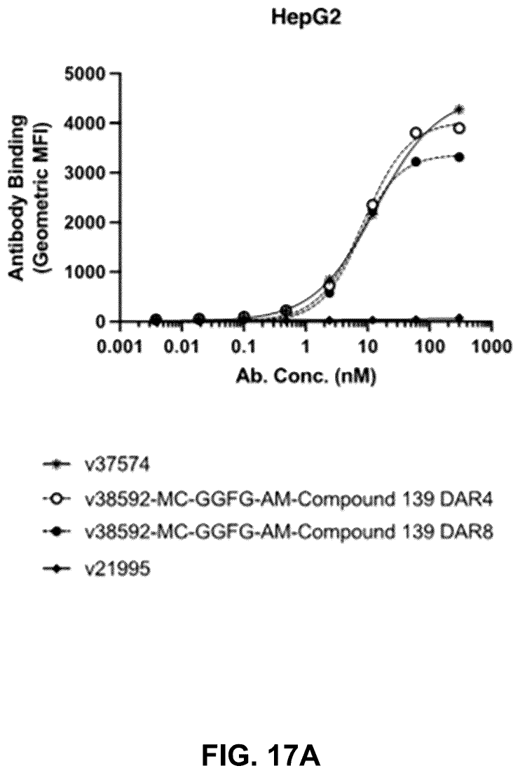

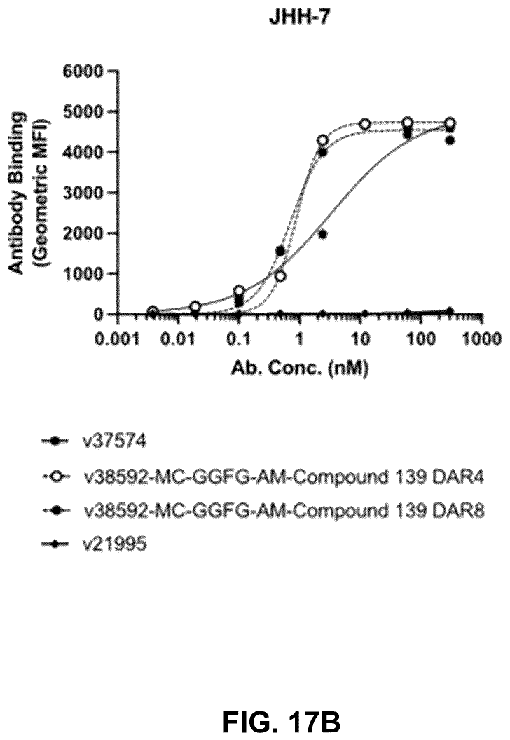

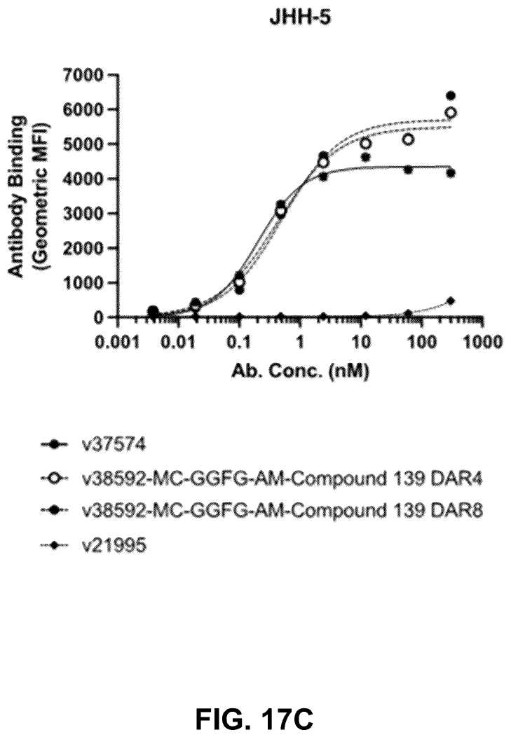

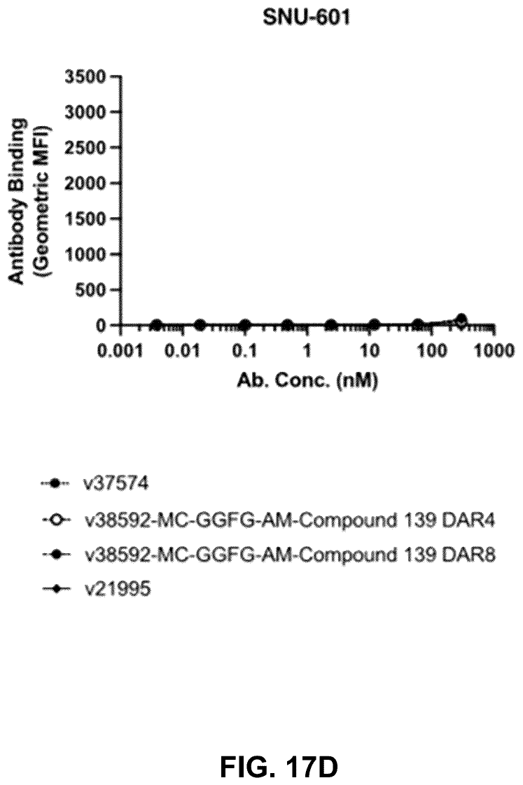

The results are shown in Table 30.1 and FIGS. 17A-17D. v37574 exhibited dose-dependent binding of GPC3-expressing cells, whereas v21995 showed minimal binding as expected. No binding was observed with any test articles in GPC3-negative SNU-601 cells. ADCs v38592-MC-GGFG-AM-Compound 139 DAR 4 and 8 showed comparable maximal binding and Kd in all GPC3-expressing cell lines tested. These results suggest that MC-GGFG-AM-Compound 139 conjugation has minimal effect on tumor binding properties.

TABLE 30.1
Cellular Binding

| | HepG2 | | JHH-7 | | JHH-5 | | SNU-601 | |
|---|---|---|---|---|---|---|---|---|
| | | Kd | | Kd | | Kd | | Kd |
| | Bmax | (nM) | Bmax | (nM) | Bmax | (nM) | Bmax | (nM) |
| v37574 | 4567 | 13.55 | 4975 | 3.31 | 4353 | 0.20 | I.C. | >300 |
| v38592-MC-GGFG-AM- | 4046 | 8.69 | 4734 | 0.88 | 5499 | 0.43 | I.C. | >300 |
| Compound 139, DAR 4 | | | | | | | | |
| v38592-MC-GGFG-AM- | 3368 | 7.27 | 4547 | 0.70 | 5709 | 0.51 | I.C. | >300 |
| Compound 139, DAR 8 | | | | | | | | |
| v21995 | I.C. | >300 | I.C. | >300 | I.C. | >300 | I.C. | >300 |

I.C.—Incomplete curve

### Example 31: In Vivo Efficacy of Anti-GPC3 M3-H18L6 ADCs in JHH-5, JHH-7, and Hep3B Liver Cancer Models

The dose-response anti-tumour activity of anti-GPC3 antibody M3-H18L6 (v38592) conjugated to MC-GGFG-AM-Compound 139 at DAR 4 or at DAR 8 were further tested in the JHH-7 and Hep3B CDX models.

In addition, the anti-tumor activity of M3-H18L6 (v37574) conjugated to MC-GGFG-AM-Compound 139 at DAR 4 or DAR 8 was investigated in the JHH-5 model. In this study, non-targeting ADC v21995-MC-GGFG-AM-Compound 139 at DAR 8 was included as a control.

Cancer cells (Table 31.1) suspended in a 1:1 mixture of PBS and Matrigel® were injected subcutaneously into the right front flank region of 8-10 week old female BALB/c nude mice. When tumours reached a mean volume of 150-200 mm³, mice were randomized into treatment groups and injected intravenously with a single dose of test article at day 0 (Table 31.2 and Table 31.3). Tumour volumes and body weights were monitored twice weekly over a 28-day study period as described in Example 22. Whole blood was collected retro-orbitally at multiple time points and processed to serum for future PK analysis. Dietary gel supplements were provided to all Hep3B mice from day 21 to day 27 and to all JHH-7 mice from day 0 to day 27.

TABLE 31.1
Characteristics of CDX Models

| | | | Inoculum and | Mean Tumour |
|---|---|---|---|---|
| | | | Volume of PBS | Volume at |
| Xenograft | Cancer | GPC3 | mixed with | Day 0 |
| Model | Type | Expression | Matrigel ® (1:1) | (mm³) |
| JHH-7 | Liver | High | 1 × 10⁷ in 0.1 mL | ~170 |
| Hep3B | Liver | High | 5 × 10⁶ in 0.1 mL | ~170 |
| JHH-5 | Liver | Mid | 1 × 10⁷ in 0.2 mL | ~150 |

TABLE 31.2
Treatment groups in the Hep3B CDX model

| | | | Dose | |
|---|---|---|---|---|
| | | | Level | |
| Group | Test Article | DAR | (mg/kg) | n |
| 1 | Vehicle, 10 mM Na acetate, | N/A | 0 | 6 |
| | 9% sucrose, pH 4.5 | | | |
| 2 | v38592-MC-GGFG-AM-Compound 139 | 4 | 2 | 6 |
| 3 | | | 4 | 6 |
| 4 | | | 6 | 6 |
| 5 | v38592-MC-GGFG-AM-Compound 139 | 8 | 2 | 6 |
| 6 | | | 4 | 6 |
| 7 | | | 6 | 6 |

TABLE 31.3
Treatment groups in the JHH-7 CDX model

| | | | Dose | |
|---|---|---|---|---|
| | | | Level | |
| Group | Test Article | DAR | (mg/kg) | n |
| 1 | Vehicle, 10 mM Na acetate, | N/A | 0 | 8 |
| | 9% sucrose, pH 4.5 | | | |
| 2 | v38592-MC-GGFG-AM-Compound 139 | 4 | 2 | 8 |
| 3 | | | 4 | 8 |
| 4 | | | 8 | 8 |
| 5 | v38592-MC-GGFG-AM-Compound 139 | 8 | 2 | 8 |
| 6 | | | 4 | 8 |
| 7 | | | 8 | 8 |

TABLE 31.3
Treatment groups in the JHH-5 CDX model

| | | | Dose | |
|---|---|---|---|---|
| | | | Level | |
| Group | Test Article | DAR | (mg/kg) | n |
| 1 | Vehicle, 10 mM Na acetate, | N/A | 0 | 8 |
| | 9% sucrose, pH 4.5 | | | |
| 2 | v37574-MC-GGFG-AM-Compound 139 | 4 | 8 | 8 |
| 3 | v37574-MC-GGFG-AM-Compound 139 | 8 | 8 | 8 |
| 4 | v21995-MC-GGFG-AM-Compound 139 | 8 | 8 | 8 |

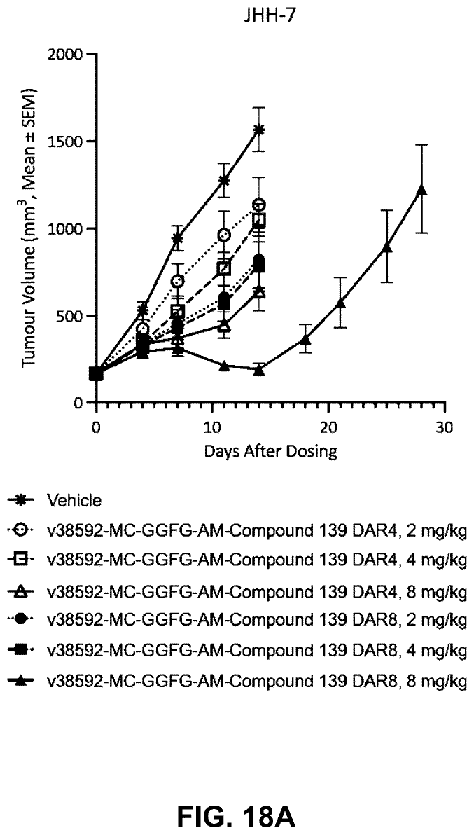

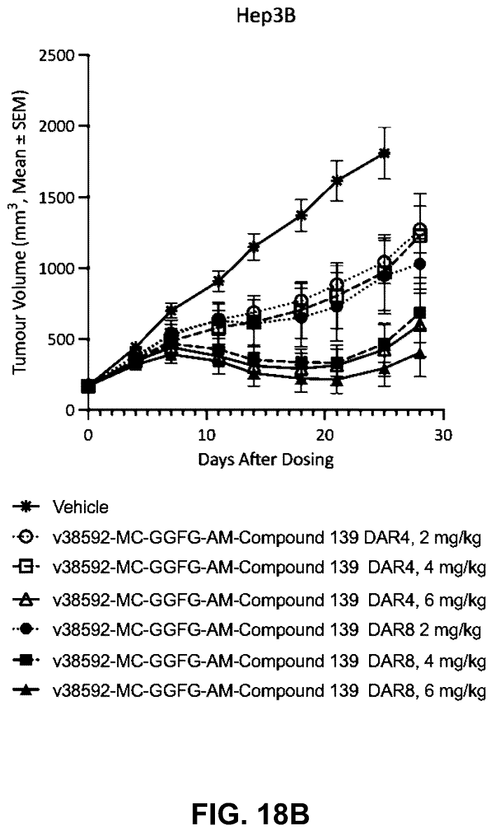

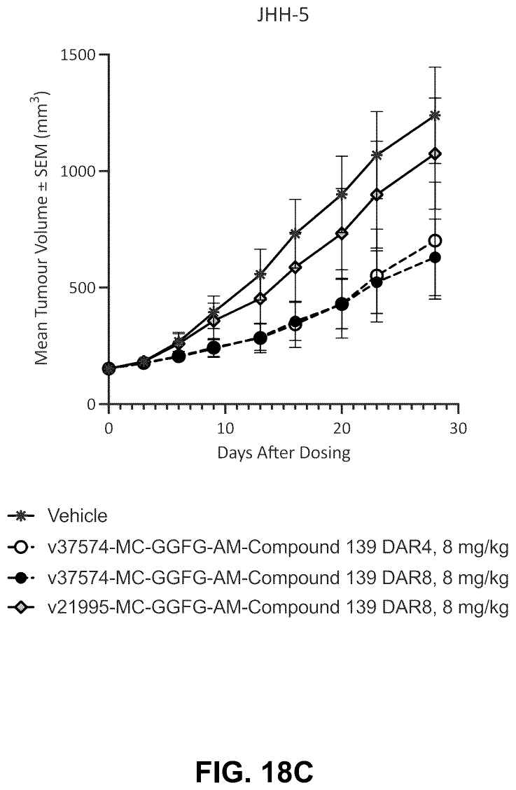

Results for the JHH-7 model are shown in FIG. 18A. Mean plot for each group was terminated when >20% of mice were lost due to tumour volumes exceeding 2000 mm³. Both ADCs demonstrated a dose-dependent increase in anti-tumor activity from 2 to 8 mg/kg. Comparable anti-tumor activity was observed between toxin matched doses as well, such as between DAR 4 ADC at 4 mg/kg and DAR 8 ADC at 2 mg/kg or between DAR4 ADC at 8 mg/kg and DAR8 ADC at 4 mg/kg.

Results for Hep3B are shown in FIG. 18B. Mean plot for each group was terminated when >20% of mice were lost due to tumour volumes exceeding 2000 mm³. Both DAR 4 and DAR 8 ADCs demonstrated a dose-dependent increase in anti-tumor activity from 2 to 6 mg/kg. Comparable anti-tumor activity was observed between the toxin matched doses of DAR 4 ADC at 4 mg/kg and DAR 8 ADC at 2 mg/kg.

Results for JHH-5 are shown in FIG. 18C. Both DAR 4 and DAR 8 ADCs demonstrated anti-tumor activity at 8 mg/kg compared to vehicle treatment or a non-targeting ADC control.

Overall, dose-dependent anti-tumor activity was observed with both DAR 4 and DAR 8 ADCs of anti-GPC3 antibody M3-H18L6 conjugated to MC-GGFG-AM-Compound 139 in JHH-7 and Hep3B CDX models. Further, anti-tumor activity of M3-H18L6 conjugated to MC-GGFG-AM-Compound 139 was demonstrated compared to a non-targeting control ADC in a JHH-5 CDX model.

### Example 32: In Vivo Efficacy of Anti-Gpc3 Antibody M3-H18L6 (V37574) ADCs in Seven Patient-Derived Xenograft (PDX) Models of Hepatocellular Carcinoma (HCC)

The anti-tumour activity of anti-GPC3 antibody M3-H18L6 (v37574) conjugated to MC-GGFG-AM-Compound 139 was investigated in seven PDX models of hepatocellular carcinoma.

The studies were carried out as follows. Tumour fragments (approximately 2 to 3 mm³) from stock mice bearing LI0050, LI1005, LI1069, LI1097, LI6610, LI6619, or LI6677 patient-derived xenografts (HuPrime® Liver Cancer Xenograft Models, Crown Bioscience Inc.) were implanted subcutaneously into 6-8 week old female BALB/c nude mice as described in Table 32.1. When tumors reached a mean volume of 150-180 mm³, mice were randomized into treatment groups and injected intravenously with a single dose of test article at day 0 as shown in Table 32.2. Tumor volumes and body weights were monitored twice weekly over a 28-day study period as described in Example 22. Dietary gel supplements were provided to all LI6677 study mice from Day 12 to the end of study.

TABLE 32.1
Characteristics of PDX Models

| | | | Mean Tumor |
|---|---|---|---|
| | | Age of Study | Volume at |
| Model | Cancer Type | Mice (weeks) | Day 0 (mm³) |
| LI0050 | Liver/HCC | 6-7 | ~170 |
| LI1005 | Liver/HCC | 6-7 | ~166 |
| LI1069 | Liver/HCC | 6-7 | ~163 |
| LI1097 | Liver/HCC | 6-7 | ~166 |
| LI6610 | Liver/HCC | 6-7 | ~150 |
| LI6619 | Liver/HCC | 7-8 | ~177 |
| LI6677 | Liver/HCC | 6-7 | ~163 |

TABLE 32.2
Treatment groups in each PDX model

| | | Dose | |
|---|---|---|---|
| | | Level | |
| Group | Test Article | (mg/kg) | n |
| 1 | Vehicle, 10 mM Na acetate, 9% sucrose, pH 4.5 | 0 | 3 |
| 2 | v37574-MC-GGFG-AM-Compound 139 DAR4 | 8 | 3 |
| 3 | v37574-MC-GGFG-AM-Compound 139 DAR8 | 8 | 3 |
| 4 | v21995-MC-GGFG-AM-Compound 139 DAR8 | 8 | 3 |

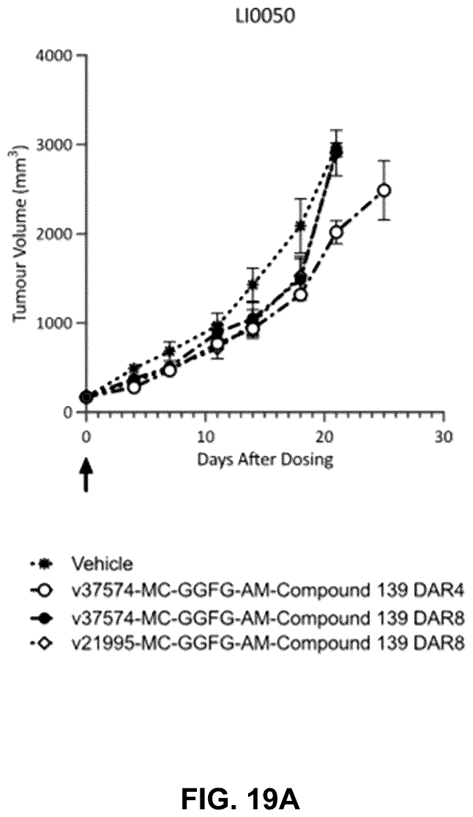

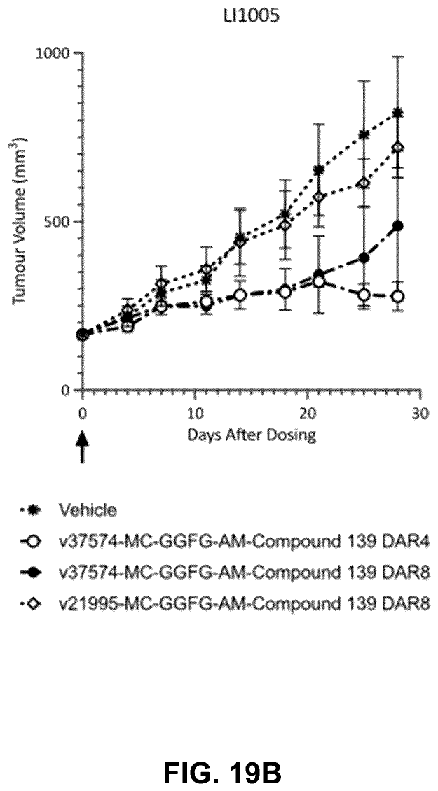

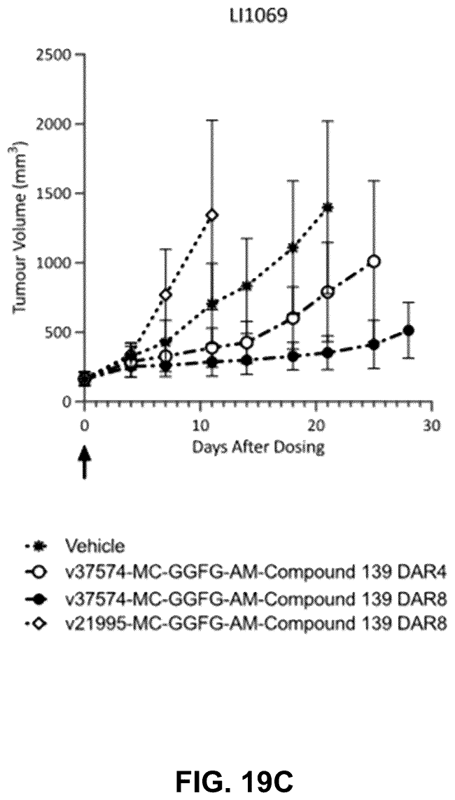

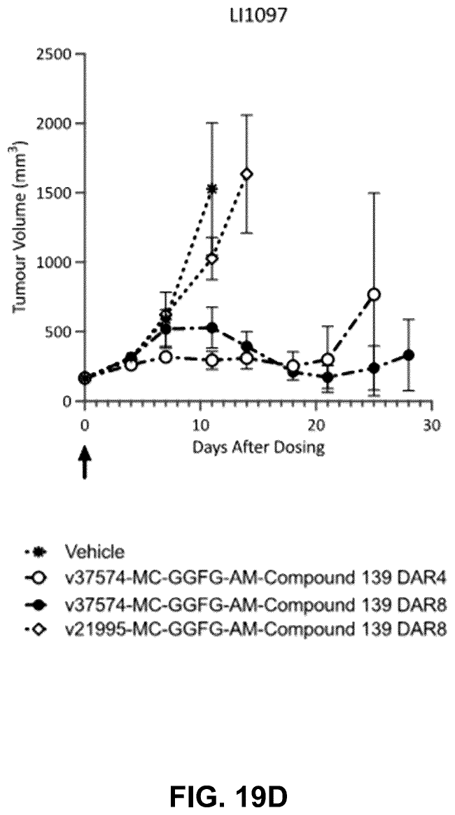

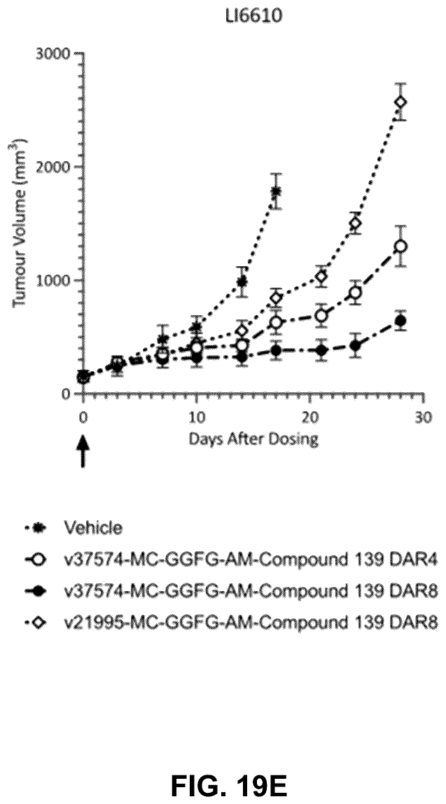

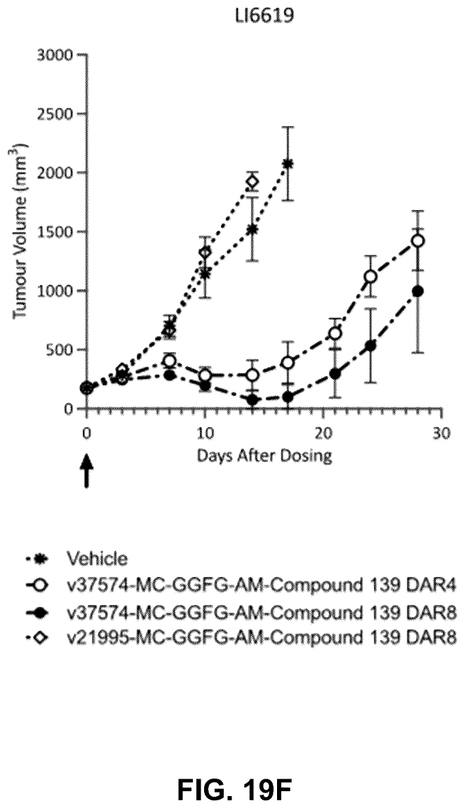

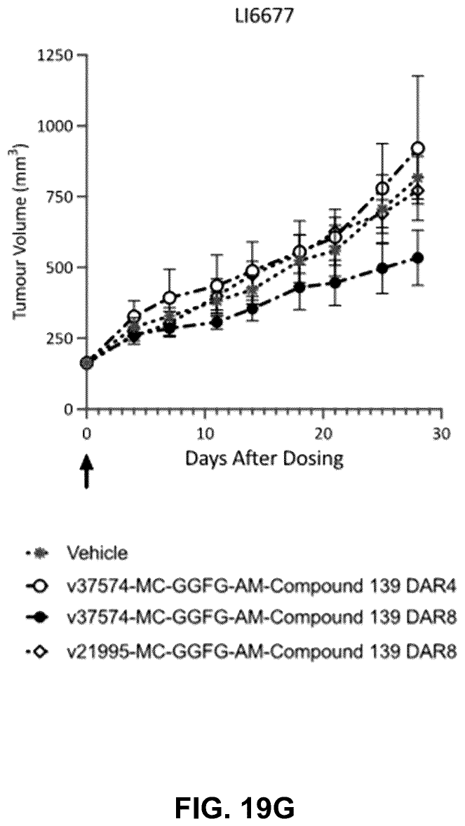

Results for LI0050 are shown in FIG. 19A. DAR4 and DAR8 v37574 ADCs showed minimal anti-tumor activity relative to non-targeting or vehicle controls.

Results for LI1005 are shown in FIG. 19B. DAR4 and DAR8 v37574 ADCs showed anti-tumor activity. At the end of the study period (day 28), 3 of 3 mice treated with DAR4 v37574 ADC and 2 of 3 mice treated with DAR8 v37574 ADC showed a delay in tumor growth. No substantial anti-tumor activity was observed with the non-targeting control (v21995) ADC.

Results for LI1069 are shown in FIG. 19C. DAR4 and DAR8 v37574 ADCs showed anti-tumor activity. At day 28, 2 of 3 mice treated with DAR4 v37574 ADC and 3 of 3 mice treated with DAR8 v37574 ADC showed tumor growth inhibition relative to the control groups. No substantial anti-tumor activity was observed with the non-targeting control ADC.

Results for LI1097 are shown in FIG. 19D. DAR4 and DAR8 v37574 ADCs showed anti-tumor activity. At day 28, 2 of 3 mice treated with DAR4 v37574 ADC and 2 of 3 mice treated with DAR8 v37574 ADC showed tumor regression. No substantial anti-tumor activity was observed with the non-targeting control ADC.

Results for LI6610 are shown in FIG. 19E. DAR4 and DAR8 v37574 ADCs showed anti-tumor activity. At day 28, all mice treated with DAR4 v37574 ADC and DAR8 v37574 ADC showed tumor growth delay compared to non-targeting control. Weak anti-tumor activity was observed with the non-targeting control ADC relative to vehicle control.

Results for LI6619 are shown in FIG. 19F. DAR4 and DAR8 v37574 ADCs showed anti-tumor activity. At day 28, 3 of 3 mice treated with DAR4 v37574 ADC exhibited tumor growth delay relative to controls, and 2 of 3 mice treated with DAR8 v37574 ADC showed tumor delay following a period of tumor regression. One mouse treated with DAR8 37574 ADC showed complete tumor regression at study termination. No substantial anti-tumor activity was observed with the non-targeting control ADC.

Results for LI6677 are shown in FIG. 19G. DAR4 and DAR8 v37574 ADCs showed minimal anti-tumor activity relative to non-targeting or vehicle controls.

Overall, v37574 ADCs demonstrated in vivo efficacy in several GPC3-expressing PDX models of hepatocellular carcinoma.

### Example 33: Assessment of GPC3 Expression and Anti-Tumor Responses in CDX/PDX Models of HCC by Immunohistochemistry (IHC)

In order to characterize and compare GPC3 expression in the CDX and PDX xenograft studies described in Examples 25, 26, 31, and 32, GPC3 expression in tumor samples sourced from these studies was assessed by immunohistochemistry (IHC). IHC was carried out as follows.

Humanized reference anti-GPC3 antibody codrituzumab (v37575) was labelled with digoxigenin using the Human-on-Human HRP-Polymer kit (Biocare Medical Cat #BRR4056KG). Tissues were fixed in 10% neutral buffered formalin for 24 h at room temperature, stored in 70% ethanol and then processed into paraffin blocks. Formalin-fixed paraffin embedded tissues were cut into 4 μm-thick sections and mounted onto Superfrost Plus glass slides (Fisher Scientific Cat #12-550-15). Sections were deparaffinized with xylene and rehydrated in decreasing concentrations of alcohol. Slides were submerged in antigen retrieval solution (Diva Decloaker, Biocare Medical Cat #DV2004) and heated in a Decloaking Chamber (Biocare Medical, Model DC2008US) to 110° C. for 15 min. Slides were cooled at room temperature for 10 min and washed with dH20. Tissue sections were delimited with a Super Pap Pen and rinsed with TBS buffer containing 0.05% (v/v) Tween-20 (TBST). Subsequent blocking and staining steps were performed at room temperature using the intelliPATH FLX™ autostainer. Slides were washed with TBST between incubations. Tissue sections were blocked with Peroxidazed 1 (Biocare Medical Cat #PX968) for 5 min, washed, and then blocked with Background Punisher (Biocare Medical Cat #BP974) for 10 min. Sections were stained with digoxigenin-labelled v37575 (0.5 μg/mL) for 30 min, washed, and then incubated with 1 μg/mL rabbit anti-digoxigenin (R&D Systems Cat #MAB10386-SP) for 30 min. After washing with TBST, sections were incubated with MACH4 Universal HRP Polymer (Biocare Medical Cat #M4U534G) for 30 min, washed, and incubated with intelliPATH FLX™ DAB Chromogen (Biocare Medical Cat #IPK5010G80) for 5 min. Tissue sections were counterstained with CAT Hematoxylin (Biocare Medical Cat #CATHE-M) diluted 1:5 in dH20 for 5 min, rinsed with dH20, and then cover-slipped with EcoMount (Biocare Medical Cat #EM897L). Whole slide scans were acquired with an Aperio Versa (Leica) slide scanner and images were collected via Aperio ImageScope (Version 12.4.3.5008). Representative fields were reviewed by pathologist and given an H-score calculated using the following equation: [(1×% IHC1+cells)+(2×% IHC2+cells)+(3×% IHC3+cells)]. Average H-scores for each sample are listed in Table 33A.

In conjunction, the in vivo anti-tumor activity of a single administration of 8 mg/kg anti-GPC3 antibody M3-H18L6 (v37574 and v38592) conjugated to MC-GGFG-AM-Compound 139 at DAR 4 or DAR 8 was determined. Anti-tumor effect in the CDX and PDX models described in Examples 25, 26, 31, and 32 was determined by % tumor growth inhibition (% TGI) calculated as [(1-mean tumor volumetreatment/mean tumor volumevehicle)×100] at study day 21 post-treatment, or at the closest evaluable time point as indicated.

The averaged H-scores of each xenograft model, and the % TGI of each ADC, is listed in Table 33A. A range of GPC3 expression was observed across the 6 CDX and 9 PDX models investigated, with H-scores ranging from 84 to 300. Tumor growth inhibition was observed with a single 8 mg/kg dose of either DAR 4 or DAR 8 conjugates of anti-GPC3 antibody M3-H₁₈L6-MC-GGFG-AM-Compound 139 in nearly all models tested. Table 33B outlines the relationship between ADC anti-tumor activity and GPC3 expression in HCC tumor xenografts, where positive anti-tumor activity was defined as treatment resulting in % TGI equal or greater than 50%. Both DAR 4 and DAR 8 ADCs demonstrated positive anti-tumor activity in 82% of HCC models with a GPC3 expression greater than H-score 200. In HCC models with GPC3 expression less than H-score 200, the DAR 4 and DAR 8 ADCs exhibited positive anti-tumor activity in 50% and 75% of models, respectively. Overall, these results suggest that anti-GPC3 antibody M3-H₁₈L6-MC-GGFG-AM-Compound 139 conjugates demonstrate broad activity in HCC CDX/PDX models, including those with lower or heterogenous GPC3 expression.

TABLE 33A
In vivo tumor growth inhibition with a single dose of 8 mg/kg anti-GPC3
antibody M3-H18L6-MC-GGFG-AM-Compound 139 in CDX and PDX tumor models

| | | | Tumor growth inhibition | | | |
|---|---|---|---|---|---|---|
| | | | v37574-MC- | v37574-MC- | v38592-MC- | v38592-MC- |
| | | | GGFG-AM- | GGFG-AM- | GGFG-AM- | GGFG-AM- |
| | | Study | Compound | Compound | Compound | Compound |
| | H- | day | 139 | 139 | 139 | 139 |
| Model | score | evaluated | DAR 4 | DAR 8 | DAR 4 | DAR 8 |
| JHH-5 | 300 | 20 | 52% | 52% | | |
| HepG2 | 278 | 20 | 94% | 92% | | |
| Hep3B | 267 | 21 | 91% | 93% | | |
| Huh-7 | 265 | 21 | 94% | 95% | | |
| JHH-7 | 198 | 14 | | | 59% | 88% |
| PLC/PRF/5 | 138 | 21 | 43% | 60% | | |
| LI1097 | 300 | 11 | 81% | 65% | | |
| LI1069 | 300 | 21 | 43% | 75% | | |
| LI1005 | 295 | 21 | 51% | 48% | | |
| LI1025 | 288 | 21 | 88% | 91% | | |
| LI0050 | 250 | 14 | 34% | 26% | | |
| LI6619 | 248 | 17 | 81% | 95% | | |
| LI1037 | 225 | 10 | 93% | 96% | | |
| LI6677 | 142 | 21 | −9% | 20% | | |
| LI6610 | 84 | 17 | 65% | 79% | | |

TABLE 33B
Frequency of in vivo anti-GPC3 antibody M3-
H18L6-MC-GGFG-AM-Compound 139 anti-tumor
activity in relation to GPC3 expression

| | Frequency of M3-H18L6-MC-GGFG-AM-Compound | |
|---|---|---|
| CDX/PDX | 139 anti-tumor activity | |
| H-score | DAR 4 | DAR 8 |
| <200 | 50% (2/4) | 75% (3/4) |
| >200 | 82% (9/11) | 82% (9/11) |

### Example 34: Cynomolgus Monkey Maximum Tolerated Dose Toxicity Study

The objective of this study was to determine the maximum tolerated dose of anti-GPC3 antibody M3-H18L6 (v38592) conjugated to MC-GGFG-AM-Compound 139 at DAR4 or DAR8 in male cynomolgus monkeys following three repeat slow intravenous bolus injections. In addition, the toxicokinetic (TK) profiles of these ADCs in cynomolgus monkeys was characterized.

In this study, vehicle and test article ADCs were administered by slow intravenous injection over 3 minutes on Day 1, Day 22, and Day 43 to male cynomolgus monkeys (n=3/group). Study design, dose levels and dose volume details are summarized in Table 34.1. All the animals were evaluated for moribundity/mortality, clinical signs, body weight, food consumption, clinical pathology (hematology, serum chemistry and coagulation), changes in organ weight, and macroscopic and microscopic changes in organs/tissues. Blood samples were collected for toxicokinetic analysis following the first dose. The test article concentrations in all dose formulations were analyzed using UV-Vis assay. Scheduled necropsy was conducted on study Day 50.

TABLE 34.1
Study Design

| | | | | Dose | | |
|---|---|---|---|---|---|---|
| | | | Dose level | volume | Concentration | Number of |
| Group | Treatment | DAR | (mg/kg/dose) | (mL/kg) | (mg/mL) | animals |
| 1 | Vehicle | | 0 | 5 | 0 | 3 |
| 2 | v38592-MC-GGFG- | 4 | 20 | 5 | 4 | 3 |
| 3 | AM-Compound 139 | | 60 | 5 | 12 | 3 |
| 4 | | | 120 | 5 | 24 | 3 |
| 5 | v38592-MC-GGFG- | 8 | 10 | 5 | 2 | 3 |
| 6 | AM-Compound 139 | | 30 | 5 | 6 | 3 |
| 7 | | | 60 | 5 | 12 | 3 |

### Results

v38592-MC-GGFG-AM-Compound 139, DAR4: All animals survived to their scheduled euthanasia (Study Day 50). No abnormal functional observational battery observations were noted. treatment-related but non-adverse cage-side clinical observations included loose feces intermittently throughout the study at 120 mg/kg/day.

Preterminal Animals: Mean body weight gain and mean body weights were comparable to controls and no effect on qualitative food consumption was noted. As compared to animal baseline values and/or historical control data, fibrinogen (FIB) and lactate dehydrogenase (LDH) levels were transiently increased on Day 4 at all dose levels; however, values returned to baseline throughout the remainder of the study.

Terminal Animals: A single animal administered 120 mg/kg/dose was noted with the macroscopic observation of decreased thymus size. Dose-responsive decrease in absolute, organ to body weight, and organ to brain weight ratios were noted in the thymus of animals administered 20, 60, or 120 mg/kg/dose.

v38592-MC-GGFG-AM-Compound 139, DAR8: All animals survived to their scheduled euthanasia (Study Day 50). No abnormal functional observational battery observations were noted. Treatment-related but non-adverse cage-side clinical observations included loose feces intermittently throughout the study at 60 mg/kg/day.

Preterminal Animals: Mean body weight gain and mean body weights were comparable to controls and no effect on qualitative food consumption was noted. As compared to animal baseline values and/or historical control data, fibrinogen (FIB) and lactate dehydrogenase (LDH) levels were transiently increased on Day 4; however, values returned to baseline throughout the remainder of the study.

Terminal Animals: A single animal administered 60 mg/kg/dose was noted with the macroscopic observation of unilateral epididymis agenesis. Dose-responsive decrease in absolute, organ to body weight, and organ to brain weight ratios were noted in the thymus of animals administered 10, 30, or 60 mg/kg/dose.

### Detailed Summary of Results at End of Study

v38592-MC-GGFG-AM-Compound 139, DAR4: All animals survived their scheduled euthanasia (Study Day 50). No abnormal functional observational battery observations were noted. treatment-related but non-adverse cage-side clinical observations included loose feces intermittently throughout the study at 120 mg/kg/day. Treatment-related, non-adverse lower mean body weight was observed following each dose administration. At the end of the dosing phase (Study Day 50), mean body weight was 5.80% lower as compared to controls for animals administered 120 mg/kg/dose. Treatment-related, non-adverse decreased reticulocyte counts were observed intermittently throughout the study at 20, 60, or 120 mg/kg/dose. No treatment-related effects on organ weights were observed. Test article-related, non-adverse macroscopic observation of decreased thymus size (with correlating microscopic observation of decreased cellularity) and decreased cellularity in the mesenteric lymph node was observed in a single male administered 120 mg/kg/dose. Based upon these data, the MTD is considered to be 120 mg/kg/dose.

v38592-MC-GGFG-AM-Compound 139, DAR8. All animals survived their scheduled euthanasia (Study Day 50). No abnormal functional observational battery observations were noted. treatment-related but non-adverse cage-side clinical observations included loose and soft feces intermittently throughout the study at 60 mg/kg/day. Treatment-related, non-adverse lower mean body weight was observed following each dose administration. At the end of the dosing phase (Study Day 50), mean body weight was 6.4600 lower as compared to controls for animals administered 60 mg/kg/dose. Treatment-related, non-adverse decreased reticulocyte counts were observed intermittently throughout the study at 10, 30, or 60 mg/kg/dose. No treatment-related effects on organ weights or macroscopic or microscopic observations were noted. Based upon these data, the MTD is considered to be 60 mg/kg/dose.

This study indicated that the test articles were well tolerated in non-human primates at the doses tested.

TABLE 35.1
NHP 3-Dose Non-GLP Toxicology Study, Q3W × 3

| | Dose | | Clinical | Histo- | Clinical | | |
|---|---|---|---|---|---|---|---|
| Test Article | mg/kg | Mortality | Observations | pathology | Chemistry | Hematology | MTD |
| v38592-MC- | 20 | None | None | None | None | Decreased | 120 |
| GGFG-AM- | | | | | | reticulocyte | |
| Compound | | | | | | counts (Day | |
| 139, DAR4 | | | | | | 46) | |
| | 60 | None | None | None | None | Decreased | |
| | | | | | | reticulocyte | |
| | | | | | | counts (Days | |
| | | | | | | 4, 46, 50) | |
| | 120 | None | Loose feces | Decreased | None | Decreased | |
| | | | | cellularity | | reticulocyte | |
| | | | | (Thymus and | | counts (Days | |
| | | | | mesenteric | | 4, 8, 46, 50) | |
| | | | | lymph node) | | | |
| v38592-MC- | 10 | None | None | None | None | Decreased | 60 |
| GGFG-AM- | | | | | | reticulocyte | |
| Compound | | | | | | counts (Day | |
| 139, DAR8 | | | | | | 46) | |
| | 30 | None | None | None | None | Decreased | |
| | | | | | | reticulocyte | |
| | | | | | | counts (Days | |
| | | | | | | 4, 46, 50) | |
| | 60 | None | Loose and | None | None | Decreased | |
| | | | soft feces | | | reticulocyte | |
| | | | | | | counts (Days | |
| | | | | | | 4, 46, 50) | |

### Example 35: Cynomolgus Monkey Pharmacokinetics Study

This example describes additional methods and results from the study described in Example 34, related to characterizing the pharmacokinetic profiles of ADCs v38592-MC-GGFG-AM-Compound 139 (DAR4) and v38592-MC-GGFG-AM-Compound 139 (DAR8) in male cynomolgus monkeys following three repeat slow intravenous bolus injections.

As indicated in Example 34, vehicle and test article ADCs were administered by slow intravenous injection over 3 minutes on Day 1, Day 22, and Day 43 to male cynomolgus monkeys (n=3/group) followed by scheduled necropsy on Day 50. Detailed study design, dose levels and dose volume details are described in Example 34.

Test articles concentrations in cynomolgus monkey serum samples were measured by a sandwich ELISA utilizing a non-NIP-cross-reactive recombinant anti-human IgG antibody (Abcam plc, Cambridge, UK; Cat. ab124055) and an HRP-conjugated Goat anti-Human IgG (H+L) detection antibody (Novus International, Inc, Chesterfield, MO; Cat. NB7489) for total antibody levels. Absorbance at 450 nm was measured using a Synergy™ H1 Hybrid Multi-Mode Plate Reader (BioTek Instruments, Winooski, VT). Sample data were analyzed using SoftMax® Pro 7.1 (Molecular Devices, San Jose, CA). Individual animal pharmacokinetics parameters were calculated from non-compartmental analysis using Phoenix WinNonlin™ software (Certara, Princeton, NJ), and group mean was reported.

### Results

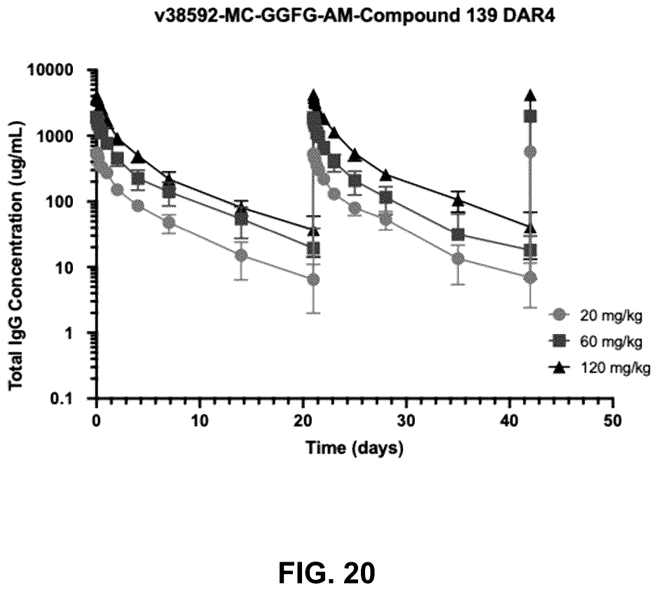

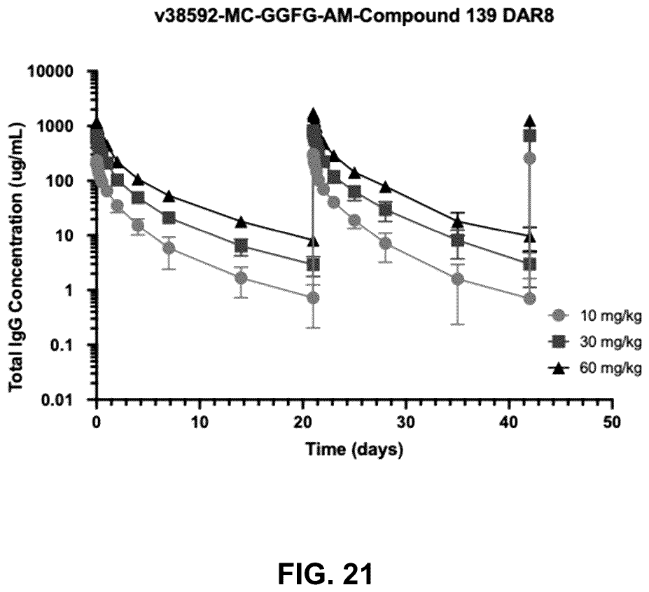

Pharmacokinetic (PK)profile: The PK profiles obtained are shown in FIG. 20 for v38592-MC-GGFG-AM-Compound 139 DAR4 and in FIG. 21 v38592-MC-GGFG-AM-Compound 139 DAR8. All ADCs assessed demonstrated a typical antibody PK profile. Across first and second dose, both DAR4 and DAR8 ADCs demonstrated PK profiles with typical antibody-like prolonged exposures. Dose proportionality for v38592-MC-GGFG-AM-Compound 139 DAR4 and DAR8 ADCs was observed across all three dosing levels.

Values for total antibody Cmax, AUCₗₐₛₜ, and ADC half-lives are noted in Table 35.1 and Table 35.2. Geometric mean was reported for Cmax and AUCₗₐₛₜ, while arithmetic mean was reported for t_{1/2}. Overall, dose proportionality was observed in both DAR4 and DAR8 ADCs following the first and second dose, with minimal dose accumulation observed at each dose.

TABLE 35.1
First Dose PK Parameter Summary

| | Dose | Cmax | AUCₗₐₛₜ | t½ |
|---|---|---|---|---|
| Test Article | (mg/kg) | (μg/mL) | (day*μg/mL) | (days) |
| v38592-MC- | 20 | 556 | 1262 | 4.6 |
| GGFG-AM- | 60 | 1947 | 3822 | 4.8 |
| Compound 139, | 120 | 4002 | 7716 | 5.4 |
| DAR4 | | | | |
| v38592-MC- | 10 | 235 | 268 | 4.4 |
| GGFG-AM- | 30 | 678 | 857 | 4.7 |
| Compound 139, | 60 | 1190 | 1869 | 5.0 |
| DAR8 | | | | |

TABLE 35.2
Second Dose PK Parameter Summary

| | Dose | Cmax | AUCₗₐₛₜ | t½ |
|---|---|---|---|---|
| Test Article | (mg/kg) | (μg/mL) | (day*μg/mL) | (days) |
| v38592-MC- | 20 | 533 | 1173 | 4.3 |
| GGFG-AM- | 60 | 1885 | 3208 | 3.7 |
| Compound 139, | 120 | 4242 | 8342 | 5.2 |
| DAR4 | | | | |
| v38592-MC- | 10 | 306 | 313 | 3.2 |
| GGFG-AM- | 30 | 819 | 1030 | 4.0 |
| Compound 139, | 60 | 1727 | 2375 | 4.0 |
| DAR8 | | | | |

### Example 36: Production of Light Chain-Modified M3-H18L6 Anti-GPC3 Antibodies

Assessment of v37574 and v38592 (antibody constructs based on anti-GPC3 antibody M3-H₁₈L6) for deamidation at asparagine residues was performed according to standard methods (for example, the method described in Example 44). It was determined that deamidation at N33 (Kabat residue 28) of LCDR1 did occur, resulting in some loss of binding activity to GPC3 (data not shown). To mitigate deamidation at this residue, light chain (LC)-modified constructs were produced in full-size antibody (FSA) format containing two identical full-length heavy chains and two identical kappa light chains, as described in Example 6. Modifications in the light chain consisted of point mutations in the N33 (Kabat 28) or G34 (Kabat 29) residues in the LC CDR1 region. Amino acid residues for such substitutions were selected with the goal of primarily eliminating deamidation as a liability at position 33 or reducing/eliminating deamidation at N33 by substitution at adjacent residue G34. The following LC-modified antibody constructs were constructed: H₁₈L6_LC_G34D mAb, H₁₈L6_LC_G34E mAb, H₁₈L6_LC_G34H mAb, H₁₈L6_LC_G34R mAb, H₁₈L6_LC_G34K mAb, H₁₈L6_LC_G34Q mAb, H₁₈L6_LC_G34T mAb, H₁₈L6_LC_G34S mAb, H₁₈L6_LC_G34V mAb, H₁₈L6_LC_G34A mAb, H₁₈L6_LC_N₃₃D mAb, H₁₈L6_LC_N₃₃Q mAb, H₁₈L6_LC_N₃₃S mAb, H₁₈L6_LC_N₃₃E mAb, H₁₈L6_LC_N₃₃T mAb, H₁₈L6_LC_N₃₃V mAb, H₁₈L6_LC_N₃₃R mAb, H₁₈L6_LC_N₃₃K mAb, H₁₈L6_LC_N₃₃H mAb, H₁₈L6_LC_N₃₃A mAb. These antibody constructs and their corresponding variant numbers are shown in Table 36.1.

TABLE 36.1
LC-modified antibody constructs
and corresponding variant number

| Variant number | Antibody construct |
|---|---|
| v38592 | M3-H18L6 (WT) mAb |
| v40203 | H18L6_LC_G34D mAb |
| v40204 | H18L6_LC_G34E mAb |
| v40205 | H18L6_LC_G34H mAb |
| v40206 | H18L6_LC_G34R mAb |
| v40207 | H18L6_LC_G34K mAb |
| v40208 | H18L6_LC_G34Q mAb |
| v40209 | H18L6_LC_G34T mAb |
| v40210 | H18L6_LC_G34S mAb |
| v40211 | H18L6_LC_G34V mAb |
| v40212 | H18L6_LC_G34A mAb |
| v40213 | H18L6_LC_N33E mAb |
| v40214 | H18L6_LC_N33T mAb |
| v40215 | H18L6_LC_N33V mAb |
| v40216 | H18L6_LC_N33R mAb |
| v40217 | H18L6_LC_N33K mAb |
| v40218 | H18L6_LC_N33H mAb |
| v40219 | H18L6_LC_N33A mAb |
| v40220 | H18L6_LC_N33Q mAb |
| v40221 | H18L6_LC_N33S mAb |
| v40222 | H18L6_LC_N33D mAb |

The amino acid sequences of the VH and VL regions for 3 of the G34 LC-modified antibody constructs are provided in Table 36.2:

TABLE 36.2
VH and VL sequences of Light Chain Modified M3-H18L6 mAb Constructs

| Antibody | |
|---|---|
| Construct | VH and VL Sequences of Antibody Construct |
| H18L6_LC_G34 | VH (SEQ ID NO: 29) |
| RmAb | QVQLVQSGAEVKKPGSSVKVSCKASGYTFTDYEMHWVKQAPGH |
| | GLEWIGAIDPETGDTAYNQKFKGKATLTADTSTSTAYMELSSLRS |
| | EDTAVYYCTRYFSFAYWGQGTLVTVSS |
| | VL (SEQ ID NO: 68) |
| | EIVLTQSPATLSLSPGERATISCRSSQSLRHSNRNTYLQWYQQKPG |
| | QAPRLLIYKVSNRFSGVPARFSGSGSGTDFTLTISSLEPEDFAVYFC |
| | YQSKHVPYTFGGGTKVEIK |
| H18L6_LC_G34 | VH (SEQ ID NO: 29) |
| K mAb | QVQLVQSGAEVKKPGSSVKVSCKASGYTFTDYEMHWVKQAPGH |
| | GLEWIGAIDPETGDTAYNQKFKGKATLTADTSTSTAYMELSSLRS |
| | EDTAVYYCTRYFSFAYWGQGTLVTVSS |
| | VL (SEQ ID NO: 64) |
| | EIVLTQSPATLSLSPGERATISCRSSQSLRHSNKNTYLQWYQQKPG |
| | QAPRLLIYKVSNRFSGVPARFSGSGSGTDFTLTISSLEPEDFAVYFC |
| | YQSKHVPYTFGGGTKVEIK |
| H18L6_LC_G34 | VH (SEQ ID NO: 29) |
| Q mAb | QVQLVQSGAEVKKPGSSVKVSCKASGYTFTDYEMHWVKQAPGH |
| | GLEWIGAIDPETGDTAYNQKFKGKATLTADTSTSTAYMELSSLRS |
| | EDTAVYYCTRYFSFAYWGQGTLVTVSS |
| | VL (SEQ ID NO: 60) |
| | EIVLTQSPATLSLSPGERATISCRSSQSLRHSNQNTYLQWYQQKPG |
| | QAPRLLIYKVSNRFSGVPARFSGSGSGTDFTLTISSLEPEDFAVYFC |
| | YQSKHVPYTFGGGTKVEIK |

### 36.1 Expression and Purification of Light Chain-Modified M3-H₁₁₈L6 Antibody Constructs

The LC-modified antibody constructs described above were prepared as follows.

ExpiCHO™ cells were cultured at 37° C. in ExpiCHO™ expression medium (Thermo Fisher, Waltham, MA) on an orbital shaker rotating at 120 rpm in a humidified atmosphere of 800 CO₂. 400 mL expression volumes were used. Each 1 mL of cells at a density of 6×10⁶ cells/mL was transfected with a total of 0.8 g DNA. Prior to transfection the DNA was diluted in 76.8 μL OptiPRO™ SFM (Thermo Fisher, Waltham, MA), after which 3.2 μL of ExpiFectamine™ CHO reagent (Thermo Fisher, Waltham, MA) was directly added to make a total volume of 80 μL. After incubation for 1-5 minutes, the DNA-ExpiFectamine™ CHO Reagent complex was added to the cell culture (80 μL complex per 1 mL of cell culture) then incubated in a 120 rpm shaking incubator at 37° C. and 8% CO₂. Following incubation at 37° C. for 18-22 hours, 6 μL of ExpiCHO™ Enhancer and 240 μL of ExpiCHO™ Feed (Thermo Fisher, Waltham, MA) were added per 1 mL of culture. Cells were maintained in culture at 37° C. for a total of 8 days, after which each culture was harvested by transferring into appropriately sized centrifuge tubes and centrifuging at 4200 rpm for 15 minutes. Supernatants were filtered using a 0.2 mm polyethersulfone membrane (Thermo Fisher, Waltham, MA), then analyzed by non-reducing SDS-PAGE and Octet (ForteBio).

Protein purification was performed in either batch mode or with the use of an AKTA™ Pure purification system. For LC-modified antibody constructs with substitution at N33 only half of the supernatant (200 ml) was used in purification; for those with substitution at G34, the entire supernatant was used. In batch mode, supernatants from transient transfections were applied to slurries containing 50% MabSelect SuRe™ resin (Cytiva, Marlborough, MA) and incubated at room temperature for 1 hr on an orbital shaker at 150 rpm. The slurries were transferred into chromatography columns and supernatants were allowed to flow through while resins remained in the column. The resins were then washed with at least 5 Bed Volumes (BV) of resin Equilibration buffer (PBS). To elute the targeted proteins, 2.5 BV of Elution Buffer (100 mM sodium citrate buffer pH 3.5) was added to the columns and collected. Elutions were then neutralized by adding 16% (v/v) 1 M Tris pH 9 to reach a final pH of 5.5. In AKTA™ Pure purification mode, supernatants from transient transfections were loaded onto HiTrap MabSelect SuRE LX columns (Cytiva, Marlborough, MA) that were pre-equilibrated with 5 Column Volume (CV) of PBS. After the proteins were captured, the columns were then washed with 10 CV of PBS. The captured proteins were eluted with 5 CV of Elution Buffer (100 mM sodium citrate buffer pH 3.5) in fractions. Pooled fractions were neutralized with 16% (v/v) if 1 M Tris pH 9. Samples were then buffer exchanged into H₆NaCl buffer (20 mM L-Histidine, 50 mM NaCl pH 6.0) The protein content of each elution was determined by 280 nm absorbance measurement using a Nanodrop™.

The purity of protein samples was assessed by non-reducing and reducing LabChip™ CE-SDS. LabChip™ GXII Touch (Perkin Elmer, Waltham, MA). Analysis was carried out according to Protein Express Assay User Guide (PerkinElmer, Waltham, MA), with the following modifications. Samples at a concentration range of 5-2000 ng/μL were added to separate wells in 96 well plates (#MSP9631, BioRad, Hercules, CA) along with 7 μL of HT Protein Express Sample Buffer (#CLS920003, Perkin Elmer) and denatured at 90° C. for 5 mins. The LabChip™ instrument was operated using the LabChip™ HT Protein Express Chip (Perkin Elmer #760528) with HT Protein Express 200 assay setting.

Final yields (post Protein-A purification) for LC-modified antibody constructs with substitution at N33 ranged from 4.23 to 18.12 mg (21.15-90.6 mg/L of culture) and for LC-modified antibody constructs with substitution at G34 modified constructs final yields ranged from 6.76 to 18.71 mg (16.9-46.78 mg/L of culture). All antibodies displayed similar Caliper profiles reflective of expected antibody composition reflecting a single species corresponding to full-size antibody (NR Caliper) and intact heavy and light chains for all antibodies (R Caliper) (data not shown).

### 36.2 Quality Assessment of Antibody Constructs

Species homogeneity of the antibodies was assessed by UPLC-SEC after protein-A purification (final purification step).

Samples were analyzed as follows: UPLC-SEC was performed using an Agilent Technologies AdvanceBio SEC300 Å SEC column (7.8×150 mm, 1.7 m particles) (Agilent Technologies, Santa Clara, California) set to 25° C. and mounted on an Agilent Technologies 1260 infinity II system with a DAD detector. Run times consisted of 7 min and a total volume per injection of 5 mL with a running buffer of 200 mM K₃PO₄, 200 mM KCl, pH 7. Elution was monitored by UV absorbance in the range 190-400 nm, and chromatograms were extracted at 280 nm. Peak integration was performed using OpenLAB™ CDS ChemStation™ software. The profiles of all the tested constructs reflected high species homogeneity (data not shown).

### Example 37: Binding of Light Chain-Modified M3-H18L6 Constructs to Human and Cynomolgus GPC3

The ability of the LC-modified M3-H18L6 antibodies to bind to GPC3 was assessed by surface plasmon resonance (SPR) similarly as described in Example 7.

The SPR assay for determination of GPC3 affinity of the antibodies was carried out on a Biacore™ T200 SPR system with PBS-T (PBS+0.05% (v/v) Tween 20) running buffer (with 0.5 M EDTA stock solution added to 3 mM final concentration) at a temperature of 25° C. CM5 Series S sensor chip, Biacore™ amine coupling kit (NHS, EDC and 1 M ethanolamine), and 10 mM sodium acetate buffers were purchased from Cytiva Life Sciences (Mississauga, ON, Canada). PBS running buffer with 0.05% Tween20 (PBST) was purchased from Teknova Inc. (Hollister, CA). Antigens: recombinant human and cynomolgus GPC3 was purchased from ACROBiosystems (Newark, DE) and SEC purified on a Superdex 200 10/300 GL column (Cytiva) in PBST running buffer at 0.8 mL/min.

Screening of the antibodies for binding to GPC3 antigen was conducted via anti-Fc capture of antibodies, followed by the injection of five concentrations of GPC3. The anti-Fc surface was prepared on a CM5 Series S sensor chip by standard amine coupling methods as described by the manufacturer (Cytiva Life Sciences, Mississauga, ON, Canada). The immobilization of the anti-Fc was performed using goat anti-human IgG (Cat #109-005-098; Jackson Immuno Research, West Grove, PA) at 25 μg/mL in 10 mM sodium acetate buffer pH 4.5 and the Biacore™ T200 immobilization wizard with an amine coupling method aiming for 4000 RUs. Approximately 150-400 RUs of each antibody (5-20 μg/mL) were captured on the goat anti-human IgG surface by injecting at 10 μL/min for 60s. Using single-cycle kinetics, five concentrations of a two-fold dilution series of human or cynomolgus GPC3 starting at 40 nM, 20 nM, 10 nM, or 5 nM depending on the antibody, with a blank buffer control were injected at 20 μL/min for 180s of contact time, followed by 180 s dissociation phase, resulting in sensorgrams with a buffer blank reference. The anti-Fc surface was regenerated to prepare for the next injection cycle by one pulse of 10 mM glycine/HCl pH 1.5 for 120 s at 40 μL/min. Blank-subtracted sensorgrams were analyzed and fit to the 1:1 Langmuir binding model using Biacore™ T200 Evaluation Software v3.2.

The results for binding of antibody constructs to human GPC3 are shown in Table 37.1 and the results for binding of antibody constructs to cynomolgus GPC3 are shown in Table 37.2. As can be seen from these tables, Kd values for LC-modified antibody constructs were not significantly different to that of WT (v38592) as measured by SPR. Specifically, the data in Table 37.1 show that Kd values for the majority of LC-modified antibody constructs with modification at G34 were no more than approximately 2-fold higher compared to the Kd for M3 H18L6 (WT), while for majority of LC-modified antibody constructs with modification at N₃₃ the Kd was no more than approximately 3-4-fold higher compared to M3 H18L6 (WT), for both human and cyno GPC3 binding.

However, some differences were observed in % binding. LC-modified antibody constructs with modifications at G34 and the WT v38592 exhibited higher % binding compared to LC-modified antibody constructs with modification at N33 modification. None of the modifications introduced any significant differential affinity to human vs cyno GPC3, as can be seen from Table 37.2 where for the majority of constructs the fold difference in Kd (hu/cyno) was in ˜1.1-1.6 range.

TABLE 37.1
Assessment of Binding to huGPC3 for Light
chain-modified M3-H18L6 mAbs by SPR

| | | | Avg % binding |
|---|---|---|---|
| Variant | | Avg Kd (M) | huGPC3 (RU |
| number | Antibody Construct | huGPC3 | observed/theoretical) |
| v38592 | M3-H18L6 (WT) mAb | 7.14E−09 | 51.9 |
| v40203 | H18L6_LC_G34D mAb | 1.37E−08 | 44.1 |
| v40204 | H18L6_LC_G34E mAb | 1.49E−08 | 43.0 |
| v40205 | H18L6_LC_G34H mAb | 1.04E−08 | 43.5 |
| v40206 | H18L6_LC_G34R mAb | 1.19E−08 | 42.0 |
| v40207 | H18L6_LC_G34K mAb | 1.15E−08 | 42.2 |
| v40208 | H18L6_LC_G34Q mAb | 1.06E−08 | 46.3 |
| v40209 | H18L6_LC_G34T mAb | 1.13E−08 | 43.6 |
| v40210 | H18L6_LC_G34S mAb | 1.03E−08 | 42.8 |
| v40211 | H18L6_LC_G34V mAb | 2.10E−08 | 23.4 |
| v40212 | H18L6_LC_G34A mAb | 1.25E−08 | 42.5 |
| v40213 | H18L6_LC_N33E mAb | 2.27E−08 | 14.9 |
| v40214 | H18L6_LC_N33T mAb | 1.99E−08 | 21.3 |
| v40215 | H18L6_LC_N33V mAb | 2.25E−08 | 17.5 |
| v40216 | H18L6_LC_N33R mAb | 2.01E−08 | 15.8 |
| v40217 | H18L6_LC_N33K mAb | 1.82E−08 | 15.5 |
| v40218 | H18L6_LC_N33H mAb | 2.01E−08 | 15.6 |
| v40219 | H18L6_LC_N33A mAb | 1.83E−08 | 17.4 |
| v40220 | H18L6_LC_N33Q mAb | 1.75E−08 | 16.1 |
| v40221 | H18L6_LC_N33S mAb | 1.95E−08 | 18.0 |
| v40222 | H18L6_LC_N33D mAb | N/D | N/D |

n = 2 technical and independent repeats
N/D: Kd not determined due to low signal and poor curve fitting to 1:1 model

TABLE 37.2
Assessment of Binding to cynomolgus GPC3 for
Light chain-modified M3-H18L6 mAbs by SPR

| | | | Kd Fold |
|---|---|---|---|
| | | Avg Kd (M) | difference |
| Variant number | Antibody construct | cynoGPC3 | (hu/cyno) |
| v38592 | M3-H18L6 (WT) mAb | 4.54E−09 | 1.57 |
| v40203 | H18L6_LC_G34D mAb | 1.15E−08 | 1.20 |
| v40204 | H18L6_LC_G34E mAb | 1.25E−08 | 1.19 |
| v40205 | H18L6_LC_G34H mAb | 8.79E−09 | 1.19 |
| v40206 | H18L6_LC_G34R mAb | 7.67E−09 | 1.55 |
| v40207 | H18L6_LC_G34K mAb | 7.29E−09 | 1.58 |
| v40208 | H18L6_LC_G34Q mAb | 7.18E−09 | 1.48 |
| v40209 | H18L6_LC_G34T mAb | 7.43E−09 | 1.53 |
| v40210 | H18L6_LC_G34S mAb | 6.58E−09 | 1.56 |
| v40211 | H18L6_LC_G34V mAb | 1.85E−08 | 1.13 |
| v40212 | H18L6_LC_G34A mAb | 7.78E−09 | 1.61 |
| v40213 | H18L6_LC_N33E mAb | 1.66E−08 | 1.37 |
| v40214 | H18L6_LC_N33T mAb | 1.64E−08 | 1.21 |
| v40215 | H18L6_LC_N33V mAb | 1.78E−08 | 1.27 |
| v40216 | H18L6_LC_N33R mAb | 1.11E−08 | 1.82 |
| v40217 | H18L6_LC_N33K mAb | 1.4E−08 | 1.30 |
| v40218 | H18L6_LC_N33H mAb | 9.57E−09 | 2.10 |
| v40219 | H18L6_LC_N33A mAb | 1.04E−08 | 1.77 |
| v40220 | H18L6_LC_N33Q mAb | 1.57E−08 | 1.12 |
| v40221 | H18L6_LC_N33S mAb | 1.58E−08 | 1.24 |
| v40222 | H18L6_LC_N33D mAb | N/D | N/D |

n = 2 technical repeats
N/D: Fold change not calculated due to low signal and poor curve fitting to 1:1 model for human and cyno GPC3 binding

### Example 38: Functional Characterization of Light Chain-Modified M3-H18L6 Antibodies—Tumor Cell Binding

The on-cell binding capabilities of light chain (LC)-modified M3-H18L6 antibody constructs were assessed on JHH-7 (hepatocellular carcinoma; GPC3-high) and JHH-5 (hepatocellular carcinoma; GPC3-mid) by flow cytometry as described below. Codrituzumab (v37575) and the wild-type M3-H18L6 antibody (v38592) were used as a positive control, and palivizumab anti-RSV antibody (v21995) was used as a negative control. The assay was carried out as described in Example 13.

### Results

The results are shown in Table 38.1. In JHH-7 cells, wild-type M3-H18L6 (v38592) demonstrated dose-dependent binding with maximal binding and Kd comparable to codrituzumab. In general, N33 LC-modified antibody constructs exhibited reduced Bmax compared to wild-type antibody; similar Kd was maintained in all N33 LC-modified antibody constructs except for N₃₃V which showed a greater than 2-fold increase in Kd from wild-type. All G34 LC-modified antibody constructs showed comparable Bmax and Kd to wild-type M3-H18L6 (v38592) antibody except the G34V LC-modified antibody construct, which had an approximate 48% reduction in Bmax from wild-type. LC-modified constructs that did not show impaired binding in JHH-7 cells were further tested in JHH-5 cells.

In JHH-5 cells, wild-type M3-H18L6 (v38592) demonstrated dose-dependent binding with comparable maximal binding and Kd to codrituzumab as expected. The N₃₃E LC-modified antibody construct demonstrated impaired binding from wild-type, showing an approximate 59% reduction in Bmax and a nearly 29-fold increase in Kd. In general, the G34 LC-modified antibody constructs demonstrated comparable binding to wild-type except for G34V, which showed an approximate 37% reduction in Bmax compared to wild-type.

Negative control antibody palivizumab did not exhibit any binding to JHH-7 or JHH-5 cells, as expected.

TABLE 38.1
Cellular Binding

| | | JHH-7 | | | JHH-5 | | |
|---|---|---|---|---|---|---|---|
| | | | Curve | | | Curve | |
| | LC | Bmax | Hill | Kd | Bmax | Hill | Kd |
| Variant | Modification | (MFI) | Slope (h) | (nM) | (MFI) | Slope (h) | (nM) |
| v37575 | N/A | 14809 | 1.50 | 0.31 | 5586 | 1.12 | 0.19 |
| v38592 | N/A | 14090 | 1.30 | 0.42 | 4667 | 0.76 | 0.12 |
| v40203 | G34D | 12206 | 1.74 | 0.40 | 4276 | 1.07 | 0.24 |
| v40204 | G34E | 12515 | 1.43 | 0.42 | 4137 | 1.02 | 0.17 |
| v40205 | G34H | 12513 | 1.74 | 0.43 | 4579 | 1.07 | 0.16 |
| v40206 | G34R | 12504 | 1.72 | 0.34 | 4912 | 1.17 | 0.19 |
| v40207 | G34K | 12237 | 2.24 | 0.39 | 4745 | 1.26 | 0.20 |
| v40208 | G34Q | 11625 | 1.41 | 0.44 | 4361 | 1.19 | 0.16 |
| v40209 | G34T | 10910 | 1.70 | 0.42 | 4133 | 1.27 | 0.11 |
| v40210 | G34S | 10596 | 2.14 | 0.42 | 4136 | 1.45 | 0.10 |
| v40211 | G34V | 7415 | 1.39 | 0.40 | 2935 | 0.80 | 0.24 |
| v40212 | G34A | 13261 | 1.26 | 0.56 | 3852 | 0.87 | 0.31 |
| v40222 | N33D | N.B. | N/A | >47 | N.T. | | |
| v40220 | N33Q | 7216 | 1.04 | 0.67 | N.T. | | |
| v40221 | N33S | 7221 | 1.09 | 0.79 | N.T. | | |
| v40213 | N33E | N.T. | | | 1939 | 0.65 | 3.39 |
| v40214 | N33T | 9986 | 0.98 | 0.75 | N.T. | | |
| v40215 | N33V | 9544 | 0/86 | 1.01 | N.T. | | |
| v40216 | N33R | 9650 | 1.10 | 0.67 | N.T. | | |
| v40217 | N33K | 9227 | 1.23 | 0.56 | N.T. | | |
| v40218 | N33H | 8070 | 1.01 | 0.77 | N.T. | | |
| v40219 | N33A | 7872 | 1.06 | 0.62 | N.T. | | |
| v21995 | N/A | N.B. | N/A | >47 | N.B. | N/A | >150 |

N/A = Not applicable;
N.T. = Not tested;
N.B. = No binding

### Example 39: Functional Characterization of Light Chain-Modified M3-H18L6 Antibodies—Internalization

The receptor-mediated internalization capabilities of light chain (LC)-modified M3-H18L6 LC-modified antibody constructs, in GPC3-expressing cell lines JHH-7 (high GPC3-expressing) and JHH-5 (mid GPC3-expressing) were determined by flow cytometry as described below. Codrituzumab (v37575) and the wild-type M3-H18L6 antibody (v38592) were used as positive controls, and palivizumab anti-RSV antibody (v21995) was used as a negative control.

Briefly, antibodies were fluorescently labeled by coupling to an anti-Human IgG Fc Fab fragment AF488 conjugate (Jackson Immuno Research Labs, West Grove, PA; Cat. No. 109-547-008) at a 1:1 stoichiometric molar ratio in PBS pH 7.4 (Thermo Fisher Scientific, Waltham, MA; Cat. No. 10010-023), for 24 hours at 4° C. Cells were seeded at 50,000 cells/well in 48-well plates and incubated overnight under standard culturing conditions (37° C./5% CO₂) Gibco™ William's E Medium, GlutaMAX™ Supplement (Thermo Fisher Scientific, Waltham, MA; Cat. No. 32551020) with 10% FBS (Thermo Fisher Scientific, Waltham, MA; Cat. No. 12483020). Coupled antibodies were added to cells the following day at 5 nM and incubated under standard culturing conditions for 24 hours to allow for internalization. Following incubation, cells were dissociated, washed, and surface AF488 fluorescence was quenched using an anti-AF488 antibody (Life Technologies, Carlsbad, CA; Cat. No. A-11094) at 100 nM for 30 minutes at 4° C. Quenched AF488 fluorescence (internalized fluorescence) was detected by flow cytometry on a BD LSRFortessa™ Cell Analyzer (BD Biosciences, Franklin Lake, NJ) with 1,000 minimum events collected per well. The AF488/FITC-A GeoMean (fluorescence signal geometric mean, proportional to anti-Human Fab AF488 labelling) was calculated for the live single cell population using FlowJo™ Version 10.8.1 (BD Biosciences, Franklin Lake, NJ) and plotted for each antibody using GraphPad Prism Version 9 (GraphPad Software, San Diego, CA).

### Results

The results are shown in Table 39.1. Treatment with 5 nM of GPC3-targeting antibodies resulted in internalization in both JHH-7 and JHH-5 cells after 24 h of treatment, whereas palivizumab treatment induced minimal internalization. The N₃₃E LC-modified antibody construct exhibited approximately an 83% and 89% reduction in internalization compared to wild-type in JHH-7 and JHH-5 cells, respectively. The G34V LC-modified antibody construct resulted in an approximate 55% and 66% reduction in internalization compared to wild-type in JHH-7 and JHH-5 cells, respectively. The G34D and G34E LC-modified antibody constructs resulted in an approximate 30-40% reduction in internalization compared to wild-type in either cell line. The remaining G34 LC-modified antibody constructs exhibited comparable internalization to wild-type.

TABLE 39.1
Internalized fluorescence after antibody treatment.

| | | JHH-7 | JHH-5 |
|---|---|---|---|
| | | Internalization | Internalization |
| Antibody | Light Chain Modification | (MFI) | (MFI) |
| v37575 | N/A | 6564 | 8772 |
| v38592 | N/A | 7257 | 10466 |
| v40203 | G34D | 5011 | 6770 |
| v40204 | G34E | 4406 | 5773 |
| v40205 | G34H | 7316 | 9168 |
| v40206 | G34R | 9237 | 10673 |
| v40207 | G34K | 8757 | 10016 |
| v40208 | G34Q | 8915 | 9510 |
| v40209 | G34T | 9042 | 9281 |
| v40210 | G34S | 8901 | 8946 |
| v40211 | G34V | 3333 | 3557 |
| v40212 | G34A | 8071 | 9068 |
| v40213 | N33E | 1218 | 1202 |
| v21995 | N/A | 265 | 399 |

### Example 40: Functional Characterization of Light Chain-Modified M3-H18L6 Antibodies—Binding to Cynomolgus GPC3

The cross-reactivity of a subset of light chain (LC)-modified M3-H18L6 LC-modified antibody constructs to human and cynomolgus monkey GPC3 was assessed by flow cytometry using transfected CHO—S cells as described below. Codrituzumab (v37575) and the wild-type M3-H18L6 antibody (v38592) were used as a positive control, and palivizumab anti-RSV antibody (v21995) was used as a negative control. The subset of constructs tested were those that maintained comparable functional activity to v38592 with respect to cell binding and internalization as demonstrated in Example 38 and Example 39.

Briefly, CHO—S cells were transiently transfected for −24 hours with a pTT5-based expression plasmid encoding human GPC3 or cynomolgus monkey GPC3, 0.5 μg DNA per 1 million cells, using the Neon™ Transfection System (Thermo Fisher Scientific Corp., Waltham, MA), to transiently express human or cynomolgus monkey GPC3. Cells transfected with GFP were used as negative transfection controls. Following transfection, cells were seeded at 50,000 cells/well in V-bottom 96-well plates and treated with antibody for 24 hours at 4° C. to prevent internalization. Following incubation, cells were washed and stained with anti-human IgG Fc AF647 conjugate (Jackson Immuno Research Labs, West Grove, PA; Cat. No. 109-605-098) at 4° C. for 30 min. Following incubation and washing, fluorescence was detected by flow cytometry on a BD LSRFortessa™ Cell Analyzer (BD Biosciences, Franklin Lake, NJ) with 1,000 minimum events collected per well. AF647/APC-A GeoMean (fluorescence signal geometric mean, proportional to anti-Human AF647 binding) in live cell population was plotted against antibody concentration using GraphPad Prism Version 9 (GraphPad Software, San Diego, CA). The Bmax and Kd values were derived from the “Specific binding with Hill slope” nonlinear regression curve fit in GraphPad Prism Version 9.

The results are shown in Table 40.1. All GPC3-binding LC-modified antibody constructs exhibited comparable dose-dependent binding profiles between human GPC3−and cynomolgus GPC3-expressing CHO cells, and no binding to GFP-transfected CHO cells, as expected. All tested LC-modified antibody constructs demonstrated comparable binding to wild-type M3-H18L6 in CHO cells expressing either human or cynomolgus monkey GPC3, apart from the N₃₃E construct. The N₃₃E construct exhibited an approximately 26% reduction in Bmax compared to wild type in either cell type. Palivizumab did not exhibit any binding to any cell type, as expected.

TABLE 40.1
Binding to Human and Cynomolgus
Monkey GPC3-Expressing CHO Cells

| | LC | HuGPC3-CHO | | CynoGPC3-CHO | | GFP-CHO | |
|---|---|---|---|---|---|---|---|
| | Modi- | Bmax | Kd | Bmax | Kd | Bmax | Kd |
| Variant | fication | (MFI) | (nM) | (MFI) | (nM) | (MFI) | (nM) |
| v37575 | N/A | 2664 | 0.08 | 3276 | 0.09 | N.B. | >150 |
| v38592 | N/A | 2527 | 0.14 | 3150 | 0.19 | N.B. | >150 |
| v40203 | G34D | 2466 | 0.18 | 2872 | 0.18 | N.B. | >150 |
| v40204 | G34E | 2263 | 0.11 | 2866 | 0.15 | N.B. | >150 |
| v40205 | G34H | 2462 | 0.10 | 2992 | 0.14 | N.B. | >150 |
| v40206 | G34R | 2453 | 0.10 | 2851 | 0.13 | N.B. | >150 |
| v40207 | G34K | 2328 | 0.11 | 2928 | 0.16 | N.B. | >150 |
| v40208 | G34Q | 2282 | 0.11 | 2816 | 0.15 | N.B. | >150 |
| v40209 | G34T | 2324 | 0.11 | 2835 | 0.14 | N.B. | >150 |
| v40210 | G34S | 2361 | 0.06 | 2971 | 0.09 | N.B. | >150 |
| v40211 | G34V | 2098 | 0.11 | 2641 | 0.17 | N.B. | >150 |
| v40212 | G34A | 2201 | 0.12 | 3156 | 0.25 | N.B. | >150 |
| v40213 | N33E | 1855 | 0.25 | 2324 | 0.22 | N.B. | >150 |
| v21995 | N/A | N.B. | >150 | N.B. | >150 | N.B. | >150 |

N/A = Not applicable;
N.T. = Not tested;
N.B. = No binding

### Example 41: Preparation of Antibody-Drug Conjugates Based on M3-H18L6 LC-Modified Antibodies

Antibody-drug conjugates of the selected M3-H18L6 LC-modified antibody constructs shown in Table 41.1 were prepared. Exemplary protocols are provided below.

### v40203, 40204, 40206, 40207, 40208, 40212, 40222-MC-GGFG-AM-Compound 139

A solution of each antibody construct (10 mg) in 50 mM histidine buffer, pH 6.0 was diluted in PBS, pH 7.4 to an antibody concentration of 4.2 mg/mL. The antibody was reduced by addition of 5 mM diethylenetriamine pentaacetic acid (DTPA) (0.48 mL in PBS, pH adjusted to 7.4) and 10 mM of an aqueous tris(2-carboxyethyl)phosphine (TCEP) solution (0.019 to 0.022 mL, 2.75 to 3.20 eq). After 180 minutes at 37° C., to the antibody solution was added 347 μL of DMSO and an excess of either MC-GGFG-AM-Compound 139 (69.0 μL; 10 eq.) from a 10 mM DMSO stock solution. The conjugation reaction proceeded at room temperature with mixing for 60 minutes. An excess of 30 mM N-acetyl-L-cysteine solution (21 μL, 9 eq.) was added to quench each conjugation reaction.

TABLE 41.1
Antibody-Drug Conjugates

| Variant Number | Antibody | Drug-Linker | DAR |
|---|---|---|---|
| v40203 (G34D) | Humanized | MC-GGFG-AM-Compound 139 | 4 |
| v40204 (G34E) | | | |
| v40206 (G34R) | | | |
| v40207 (G34K) | | | |
| v40208 (G34Q) | | | |
| v40212 (G34A) | | | |
| v40222 (N33D) | | | |

### Example 42: Purification and Characterization of Antibody-Drug Conjugates

ADCs prepared as described in Example 41 were purified on an AKTA™ pure chromatography system (Cytiva Life Sciences, Marlborough, MA) using a 53 mL HiPrep 26/10 Desalting column (Cytiva Life Sciences, Marlborough, MA) and a mobile phase consisting of 10 mM NaOAc, pH 4.5 with 150 mM NaCl and a flow rate of 7.5 mL/min.

Following purification, the concentration of the ADCs was determined by measurement of absorption at 280 nm using extinction coefficients determined experimentally ADCs were also characterized by hydrophobic interaction chromatography (HIC) and size exclusion chromatography (SEC) as described below.

### 42.1.1 Hydrophobic Interaction Chromatography

Antibody and ADCs were analyzed by HIC to estimate the drug-to-antibody ratio (DAR). Chromatography was performed on an Agilent Infinity 111290 HPLC (Agilent Technologies, Santa Clara, CA) using a TSKgel® Butyl-NPR column (2.5 μm, 4.6×35 mm; TOSOH Bioscience GmbH, Griesheim, Germany) and employing a gradient of 95/5% MPA/MPB to 5/95% MPA/MPB over a period of 12 minutes at a flow rate of 0.5 mL/min (MPA=1.5 M (NH₄)₂SO₄, 25 mM NaₓPO₄, pH 7 and MPB=75% 25 mM NaₓPO₄, pH 7, 25% isopropanol). Detection was by absorbance at 280 nm.

### 42.2 Size Exclusion Chromatography

The extent of aggregation of the antibody and ADCs (˜15 μg, 5 μL injection volume) was assessed by SEC on an Agilent Infinity 111260 HPLC (Agilent Technologies, Santa Clara, CA) using an AdvanceBio SEC column (300 angstroms, 2.7 μm, 7.8×150 mm) (Agilent, Santa Clara, California) and a mobile phase consisting of 150 mM phosphate, pH 6.95 and a flow rate of 1 mL/min. Detection was by absorbance at 280 nm.

### Results

The individual contributions of the DAR0, DAR2, DAR4, DAR6 and DAR8 species to the average DAR of the purified ADCs were assessed by integration of the HPLC-HIC chromatogram. The average drug to antibody ratio (DAR) of each ADC was determined by the weighted average of each DAR species. The average DAR for each ADC, when rounded to the nearest integer, was the same as the target DAR shown in Table 42.1.

The extent of aggregation and monomer content was assessed by integration of the HPLC-SEC chromatogram. The monomer peak of each ADC was identified as the peak with the same retention time as the unconjugated antibody from which each ADC was derived from. All peaks with an earlier retention time relative to the monomer species was determined to be aggregated species. Percent monomer species determined for each ADC is shown in Table 42.1.

All ADC preparations showed >95% monomer species.

TABLE 42.1
Characterization of Prepared ADCs

| | | Target | % |
|---|---|---|---|
| Variant Number | Drug-Linker | DAR | Monomer |
| v40203 (G34D) | MC-GGFG-AM-Compound 139 | 4 | 99.5 |
| v40204 (G34E) | | 4 | 100 |
| v40206 (G34R) | | 4 | 100 |
| v40207 (G34K) | | 4 | 100 |
| v40208 (G34Q) | | 4 | 100 |
| v40212 (G34A) | | 4 | 100 |
| v40222 (N33D) | | 4 | 100 |

### Example 43: In Vitro Cytotoxicity of Light Chain-Modified M3-H18L6 Antibody-Drug Conjugates—3D Spheroid Cell Cultures

The cell growth inhibition (cytotoxicity) capabilities of light chain (LC)-modified M3-H18L6 constructs conjugated to MC-GGFG-AM-Compound 139 were assessed in cell lines HepG2 (high GPC3-expressing), NCI-H446 (mid GPC3-expressing), and SNU-601 (GPC3-negative) as described below. The ADC v38592-MC-GGFG-AM-Compound 139 was utilized as a positive control, while the ADC v21995-MC-GFG-AM-Compound 139 was utilized a negative control.

Briefly, cells were seeded in Ultra-Low Attachment 384-well plates, centrifuged and incubated at 37° C./5% CO₂ for 2 days in ATCC-recommended complete growth medium to allow for spheroid formation and growth. Monoculture cell line spheroids were then treated with a titration of test article, generated in cell growth medium RPMI-1640 (Thermo Fisher Scientific; Cat. No. 15230-162)+10% FBS (Thermo Fisher Scientific; Cat. No. 12483-020). Spheroids were incubated for a further 6 days. After incubation, CellTiter-Glo® 3D reagent (Promega Corporation, Madison, WI) was added in all wells. Plates were incubated in the dark at room temperature for 1 hour and luminescence was quantified using a BioTek Cytation 5 Cell Imaging Multi-Mode Reader (Agilent Technologies, Inc., Santa Clara, CA). The % cytotoxicity value for each treatment was calculated by the following formula: (1−(Luminescence of Treated Cells/Average Luminescence of Untreated Cells))×100. These values were plotted against test article concentration using GraphPad Prism 9 software (GraphPad Software, San Diego, CA).

The results for the 3D cytotoxicity assay are shown in Table 43.1. In brief, v38592-MC-GGFG-AM-Compound 139 showed dose-dependent targeted killing of HepG2 and NCI-H446 spheroids compared to v21995-MC-GGFG-AM-Compound 139, as expected. Minimal differentiation was observed between these two ADCs in GPC3-negative SNU-601 cells. Antibody-drug conjugates of LC-modified M3-H18L6 demonstrated comparable killing of HepG2 and NCI-H446 spheroids as v38592-MC-GGFG-AM-Compound 139, with minimal targeted killing observed in SNU-601 cells.

TABLE 43.1
Cytotoxicity of ADCs in 3D Spheroid Cell Culture

| | | | 3D EC50 | | |
|---|---|---|---|---|---|
| | LC | | | NCI- | SNU- |
| ADC | Modification | DAR | HepG2 | H446 | 601 |
| v38592-MC-GFG- | N/A | 4 | 0.89 | 0.20 | I.C. |
| AM-Compound 139 | | | | | |
| v40203-MC-GFG- | G34D | 4 | 0.72 | 0.36 | I.C. |
| AM-Compound 139 | | | | | |
| v40204-MC-GFG- | G34E | 4 | 0.60 | 0.45 | I.C. |
| AM-Compound 139 | | | | | |
| v40206-MC-GFG- | G34R | 4 | 0.60 | 0.14 | I.C. |
| AM-Compound 139 | | | | | |
| v40207-MC-GFG- | G34K | 4 | 0.51 | 0.10 | I.C. |
| AM-Compound 139 | | | | | |
| v40208-MC-GFG- | G34Q | 4 | 0.57 | 0.17 | I.C. |
| AM-Compound 139 | | | | | |
| v40212-MC-GFG- | G34A | 4 | 0.61 | 0.15 | I.C. |
| AM-Compound 139 | | | | | |
| v21995-MC-GFG- | N/A | 4 | >200 | 20.56 | I.C. |
| AM-Compound 139 | | | | | |

N/A = Not applicable;
I.C. = Incomplete curve

### Example 44: Assessment of Deamidation in Lc-Modified Anti-Gpc3 Antibodies

The objective of this experiment was to quantify deamidation by peptide mapping at residue Asn33 (N₃₃) in the light chain of wild-type v38592, and LC-modified variants v40203, v40204, v40206, v40207, v40208, v40212 after incubation in mouse plasma at 37° C. for 14 days.

Briefly, antibodies were diluted to a final concentration of 1.0 mg/ml in mouse plasma (BioIVT® MSE00PL38NC-013070) and incubated at 37° C., aiming for less than 20% v/v of antibody formulation solution in the mouse plasma dilution. Samples were removed after 0 and 14 days and stored at −80° C. until characterization. Samples were thawed at room temperature and 50 μg were incubated with 1.5 μg of recombinant EndoS endoglycosidase for one hour at room temperature. For immunoprecipitation, capture cartridges were prepared using the Agilent™ AssayMAP™ Bravo™ liquid handling platform, coupling 5 μl AssayMAP™ streptavidin cartridges (Agilent™, G5496-60010) to 50 μl of a 0.25 mg/ml solution of biotinylated goat anti-Human IgG Fc capture antibody (Jackson Immunoresearch™ 109-065-098). PBS was used as equilibration and wash solution.

After deglycosylation, plasma-incubated samples were loaded to capture cartridges using the Agilent™ AssayMAP™ Bravo™ liquid handling platform, using PBS as equilibration and wash solution. Protein was eluted with 35 μL of LC-MS grade water with 20% acetonitrile and 0.1% formic acid.

30 μL of eluate were concentrated down to dryness using an Eppendorf @Vacufuge® at 60° C. and reconstituted in 2.5 μl of LC-MS grade water for digestion. Samples were digested with the Promega™ AccuMAP™ low pH digestion kit, with manufacturer protocol “Sample Preparation Under Reducing Conditions with Urea”, scaled down half in volume, with the following modifications: antibodies v38592, v40203, v40204, v40208 and v40212 were not alkylated with iodoacemide, and underwent pre-digestion with Lys-C, followed by digestion with Lys-C and Trypsin; antibodies v40206 and v40207 were alkylated with iodoacetamide, and underwent pre-digestion with Lys-C, followed by digestion with Lys-C. Peptides of v40206 were further purified with the Agilent™ AssayMAP™ Bravo™ liquid handling platform using AssayMAP™ Reversed Phase (RP—S) cartridges (Agilent™, G5496-60033), using 50% acetonitrile and 0.1% formic acid in water as priming solution, 0.1% formic acid in water as binding and wash solution, and 25 μl of 90% acetonitrile as elution solution. Cleaned up peptides of antibodies v40206 were further digested with Glu-C(Promega, v1651) at a 1:20 mass ratio, for 4 hours at 37° C.

Digested samples were transferred to glass inserts in LC-MS vials. For LC-MS analysis, 10 μL of sample were injected into a Waters™ X-select C18 Column, 2.5 μm, 2.1 mm×150 mm using a Waters™ ACQUITY™ UPLC I-Class HPLC system coupled to a Waters™ Synapt™ G2-Si HDMS with a column temperature of 60° C. and a flow rate of 0.2 ml/min. Mobile phases consisted of A: LC-MS grade water with 0.1% v/v formic acid, and B: acetonitrile with 0.1% v/v formic acid. For each run, the column was pre-equilibrated in 1.0% mobile phase B before injection and held for 2 min at that mobile phase B %. Then, a 60 min 1 to 35% mobile phase B gradient was applied, followed by a 2 min 35 to 45% mobile phase B gradient, a 2 min 45 to 95% mobile phase B gradient, and a column wash of 2 min at 95% mobile phase B. The column was brought down to 1% mobile phase B in 4 min and then re-equilibrated to 1% mobile phase B for 3 minutes.

ESI (Electrospray ionization) was performed in positive mode with 3 kV of capillary voltage, 120° C. source temperature, 30 V sampling cone voltage, source offset of 30 V, source gas flow of 0 ml/min, desolvation temperature 350° C., cone gas flow 0 L/Hr, desolvation gas flow 800 L/hr, nebulizer gas flow 6.5 bar. Data was collected with analyzer set in resolution mode (20 000), with a m/z range from 50 to 2000 using Leu-enkephaline as Lock Mass™, and three TOF parent functions (PF1-3) with precursor selection set as “everything”. Parent MS survey in a range from 50 to 2000 m/z (PF 1-3), collision energies fixed 10 (PF1) or 30 (PF2) V and ramp high energies from 18.0 to 45.0 V (PF3) with survey scan time of 0.5 sec (PF1-3), survey interscan time of 0 (PF1-2), 0.1 (PF3) secs, MS/MS data was collected as continuum in a range of 50 to 2000 m/z (PF1-3).

Peak integration, quantification and assignments were performed in Protein Metrics Byos® v4.6 with workflow parameters using a precursor mass tolerance of 10 ppm, fragmentation type QTOF/HCD, fragment mass tolerances of 50 ppm, recalibration with Leu-Enkephalin, cleavage site RK at C-terminus, semi-specific search with 2 missed cleavages. Workflow monitored modifications were Oxidation (+15.994915 a.m.u), deamidated (+0.984016 a.m.u), Gln/Glu to Pyro-Glu (-18.010565 a.m.u), dioxidation (+31.989829 a.m.u) and N-glycan 59 common biantennary glycan library. Spectrum input options applying charges to unassigned spectra with max number of precursors per MS2 of 10, smoothing width of 0.01. A minimum score of 200 was set up as threshold for data analysis.

Deamidation quantification is reported in Table 44.1 as the percent ratio of the MS1 extracted ion chromatogram area under the curve of the deamidated peak or peaks (+0.984016 a.m.u), divided by the sum of the MS1 extracted ion chromatogram area under the curve of the deamidated peak or peaks (+0.984016 a.m.u) and the non-modified peak. LC-modified antibody constructs v40206, v40207, and v40208 showed less than half total deamidation % at residue Asn33 of the light chain after incubation in mouse plasma for 14 days at 37° C., when compared to wild-type variant v38592.

TABLE 44.1
Total deamidation % from residue Asn33 (N33) of
the light chain quantified by peptide mapping

| Antibody Sample | Day 0 | Day 14 |
|---|---|---|
| v40203 (G34D) | 2.7 ± 0.06 | 30.6 ± 0.49 |
| v40204 (G34E) | 0.0 ± 0.00 | 18.4 ± 0.49 |
| v40206 (G34R) | ND | 14.4 ± 1.20 |
| v40207 (G34K) | 0.0 ± 0.00 | 13.8 ± 0.71 |
| v40208 (G34Q) | 0.0 ± 0.00 | 13.6 ± 0.31 |
| v40212 (G34A) | 6.5 ± 0.12 | 37.3 ± 0.14 |
| v38592 (WT) | 4.8 ± 0.25 | 39.6 ± 0.79 |

ND = not determined

### Example 45: Functional Characterization of Plasma-Stressed Light Chain-Modified M3-H18L6 Antibodies—Cell Binding and Internalization

The binding and internalization capabilities of a subset of LC-modified M3-H18L6 LC antibodies after plasma stress was determined in GPC3-expressing cell lines JHH-7 (high GPC3-expressing) and JHH-5 (mid GPC3-expressing) by flow cytometry as described below. Wild-type M3-H18L6 antibody (v38592) were used as a positive control, and palivizumab anti-RSV antibody (v21995) was used as a negative control.

Briefly, plasma stress was carried out by incubating M3-H18L6 LC-modified antibodies in mouse plasma (BioIVT, Westbury, NY; Cat. No. MSE00PL38NC-0103070) for 14 days in a 37° C./5% CO₂ incubator. Functional profiles of 14-day plasma stressed antibodies were compared to 0-day controls. v21995 in PBS was used as a negative control in all assays and was not incubated in mouse plasma. For binding cells were seeded at 50,000 cells/well in V-bottom 96-well plates and treated with antibody for 24 hours at 4° C. to prevent internalization. Following incubation, cells were washed and stained with anti-Human IgG Fc AF647 conjugate (Jackson Immuno Research Labs, West Grove, PA; Cat. No. 109-605-098) at 4° C. for 30 min. Following incubation and washing, fluorescence was detected by flow cytometry on a BD LSRFortessa™ Cell Analyzer (BD Biosciences, Franklin Lake, NJ) with 1,000 minimum events collected per well. AF647/APC-A GeoMean (fluorescence signal geometric mean, proportional to anti-Human AF647 binding) in live cell population was plotted against antibody concentration using GraphPad Prism Version 9 (GraphPad Software, San Diego, CA). The Bmax, Curve Hill Slope (h) and Kd values were derived from the “Specific binding with Hill slope” nonlinear regression curve fit in GraphPad Prism Version 9. For internalization, antibodies were fluorescently labeled by coupling to an anti-Human IgG Fc Fab fragment AF488 conjugate (Jackson Immuno Research Labs, West Grove, PA; Cat. No. 109-547-008) at a 1:1 stoichiometric molar ratio in PBS pH 7.4 (Thermo Fisher Scientific, Waltham, MA; Cat. No. 10010-023), for 24 hours at 4° C. Cells were seeded at 50,000 cells/well in 48-well plates and incubated overnight under standard culturing conditions (37° C./5% CO₂) in Gibco™ William's E Medium, GlutaMAX™ Supplement (Thermo Fisher Scientific, Waltham, MA; Cat. No. 32551020) with 10% FBS (Thermo Fisher Scientific, Waltham, MA; Cat. No. 12483020). Coupled antibodies were added to cells the following day at 5 nM and incubated under standard culturing conditions for 24 hours to allow for internalization. Following incubation, cells were dissociated, washed, and surface AF488 fluorescence was quenched using an anti-AF488 antibody (Life Technologies, Carlsbad, CA; Cat. No. A-11094) at 100 nM for 30 minutes at 4° C. Quenched AF488 fluorescence (internalized fluorescence) was detected by flow cytometry on a BD LSRFortessa™ Cell Analyzer (BD Biosciences, Franklin Lake, NJ) with 1,000 minimum events collected per well. The AF488/FITC-A GeoMean (fluorescence signal geometric mean, proportional to anti-Human Fab AF488 labelling) was calculated for the live single cell population using FlowJo™ Version 10.8.1 (BD Biosciences, Franklin Lake, NJ) and plotted for each antibody using GraphPad Prism Version 9 (GraphPad Software, San Diego, CA).

The cell binding results are shown in Table 45.1. LC-modified M3-H18L6 antibody constructs demonstrated dose-dependent binding comparable to WT M3-H18L6 (v38592) in both JHH-7 and JHH-5 cells. After 14 days of incubation in mouse plasma, LC-modified antibody constructs exhibited comparable binding to respective Day 0 controls, suggesting no strong impact of mouse plasma stress on antibody binding. v21995 showed no binding to either JHH-7 and JHH-5 cells, as expected.

The internalization results are shown in Table 45.2. All LC-modified antibodies and WT M3-H18L6 demonstrated internalization above negative control v21995 in both JHH-7 and JHH-5 cell lines. Consistent with previous results, at day 0 v40203 and v40204 exhibited weaker internalization compared to WT M3-H18L6 (v38592), whereas at day 0 v40206, v40207, v40208, and v40212 showed comparable internalization to WT M3-H18L6 (v38592). All 14-day plasma-stressed samples showed a decrease in internalization compared to respective day 0 controls. WT M3-H18L6 (v38592) demonstrated a 40-50% decrease in internalization capability after mouse plasma incubation. Similarly, v40203 showed a 35-40% decrease in internalization capacity after mouse plasma incubation. In contrast, LC-modified antibody constructs v40206, v40207, and v40208 only showed a 5-20% loss in internalization capability after mouse plasma incubation. These results suggest that these LC modifications could reduce the impact of mouse plasma stress on the internalization capability of the M3-H18L6 antibody.

TABLE 45.1
Cellular binding of mouse plasma-stressed antibodies

| | | JHH-7 | | | | JHH-5 | | | |
|---|---|---|---|---|---|---|---|---|---|
| Variant | LC | Bmax (MFI) | | Kd (nM) | | Bmax (MFI) | | Kd (nM) | |
| Number | Modification | Day 0 | Day 14 | Day 0 | Day 14 | Day 0 | Day 14 | Day 0 | Day 14 |
| v38592 | N/A | 9767 | N.T. | 0.39 | N.T. | 5126 | N.T. | 0.14 | N.T. |
| v40203 | G34D | 9140 | 8065 | 0.26 | 0.62 | 4664 | 4521 | 0.11 | 0.34 |
| v40204 | G34E | 8495 | 8426 | 0.26 | 0.67 | 4756 | 4460 | 0.11 | 0.26 |
| v40206 | G34R | 9391 | 9708 | 0.15 | 0.39 | 5453 | 5319 | 0.07 | 0.21 |
| v40207 | G34K | 9625 | 10930 | 0.16 | 0.31 | 5489 | 6459 | 0.07 | 0.26 |
| v40208 | G34Q | 8911 | 11050 | 0.32 | 0.23 | 4730 | 5637 | 0.11 | 0.15 |
| v40212 | G34A | 8364 | 11321 | 0.36 | 0.28 | 4928 | 6049 | 0.15 | 0.23 |
| v21995 | N/A | 309 | N.T. | 37.7 | N.T. | N.B. | N.T. | N.B. | N.T. |

N/A = Not applicable;
N.T. = Not tested;
N.B. = No binding

TABLE 45.2
Internalized fluorescence of mouse plasma-stressed antibodies

| | | JHH-7 | | JHH-5 | |
|---|---|---|---|---|---|
| Variant | LC | Internalization (MFI) | | Internalization (MFI) | |
| Number | Modification | Day 0 | Day 14 | Day 0 | Day 14 |
| v38592 | N/A | 152 | 76 | 12179 | 7248 |
| v40203 | G34D | 104 | 63 | 8690 | 5690 |
| v40204 | G34E | 91 | 73 | 7096 | 6042 |
| v40206 | G34R | 144 | 115 | 13330 | 11062 |
| v40207 | G34K | 143 | 115 | 12770 | 11372 |
| v40208 | G34Q | 131 | 106 | 10519 | 9976 |
| v40212 | G34A | 131 | 75 | 10222 | 7291 |
| v21995 | N/A | 4 | N.T. | 506 | N.T. |

N/A = Not applicable;
N.T. = Not tested

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Modifications of the specific embodiments described herein that would be apparent to those skilled in the art are intended to be included within the scope of the following claims.

TABLE A
Clone numbers for variants

| Variant ID | H1 | H2 | L1 | L2 |
|---|---|---|---|---|
| 36180 (M3-H1L1) | CL_#27287 | CL_#27287 | CL_#26960 | CL_#26960 |
| 37574 (M3-H18L6) | CL_#28294 | CL_#28294 | CL_#28295 | CL_#28295 |
| 38592 (M3-H18L6 with HC C-terminal lysine) | CL_#29372 | CL_#29372 | CL_#28295 | CL_#28295 |
| 40206 (G34R) | CL_#28294 | CL_#28294 | CL_#30545 | CL_#30545 |
| 40207 (G34K) | CL_#28294 | CL_#28294 | CL_#30544 | CL_#30544 |
| 40208 (G34Q) | CL_#28294 | CL_#28294 | CL_#30542 | CL_#30542 |

TABLE B
DNA and amino acid sequences of clones in Table A

| | | SEQ | |
|---|---|---|---|
| | | ID | |
| Clone ID | Region | NO: | Sequence |
| CL_#27287 | HC full | 44 | EVQLVQSGAEVKKPGSSVKVSCKASGYTFTDYEMHWVRQAPGQ |
| | length | | GLEWIGAIDPETGDTAYNQKFKGRATLTADKSTSTAYMELSSLRS |
| | (amino | | EDTAVYYCTRYFSFAYWGQGTLVTVSSASTKGPSVFPLAPSSKSTS |
| | acid) | | GGTAALGCLVKDYFPEPVTVSWNSGALTSGVHTFPAVLQSSGLYS |
| | | | LSSVVTVPSSSLGTQTYICNVNHKPSNTKVDKKVEPKSCDKTHTCP |
| | | | PCPAPELLGGPSVFLFPPKPKDTLMISRTPEVTCVVVDVSHEDPEV |
| | | | KFNWYVDGVEVHNAKTKPREEQYNSTYRVVSVLTVLHQDWLNG |
| | | | KEYKCKVSNKALPAPIEKTISKAKGQPREPQVYTLPPSRDELTKNQ |
| | | | VSLTCLVKGFYPSDIAVEWESNGQPENNYKTTPPVLDSDGSFFLYS |
| | | | KLTVDKSRWQQGNVFSCSVMHEALHNHYTQKSLSLSPG |
| CL_#27287 | HC full | 45 | GAGGTGCAGCTGGTGCAGAGCGGAGCCGAGGTGAAGAAGCCC |
| | length | | GGCAGCTCCGTGAAGGTGTCTTGCAAGGCCAGCGGCTACACCT |
| | (DNA) | | TCACAGACTATGAGATGCACTGGGTGCGGCAGGCCCCCGGACA |
| | | | GGGACTGGAGTGGATCGGAGCCATCGACCCTGAGACCGGCGAT |
| | | | ACAGCCTACAACCAGAAGTTTAAGGGCAGGGCCACCCTGACAG |
| | | | CCGACAAGAGCACCTCCACAGCCTATATGGAGCTGTCTAGCCT |
| | | | GCGGTCCGAGGATACCGCCGTGTACTATTGTACAAGATACTTCT |
| | | | CTTTTGCCTATTGGGGCCAGGGCACCCTGGTGACAGTGTCCTCT |
| | | | GCTAGCACTAAGGGGCCTTCCGTGTTTCCACTGGCTCCCTCTAG |
| | | | TAAATCCACCTCTGGAGGCACAGCTGCACTGGGATGTCTGGTG |
| | | | AAGGATTACTTCCCTGAACCAGTCACAGTGAGTTGGAACTCAG |
| | | | GGGCTCTGACAAGTGGAGTCCATACTTTTCCCGCAGTGCTGCAG |
| | | | TCAAGCGGACTGTACTCCCTGTCCTCTGTGGTCACCGTGCCTAG |
| | | | TTCAAGCCTGGGCACCCAGACATATATCTGCAACGTGAATCAC |
| | | | AAGCCATCAAATACAAAAGTCGACAAGAAAGTGGAGCCCAAG |
| | | | AGCTGTGATAAAACTCATACCTGCCCACCTTGTCCGGCGCCAGA |
| | | | ACTGCTGGGAGGACCAAGCGTGTTCCTGTTTCCACCCAAGCCTA |
| | | | AAGACACCCTGATGATTTCCCGGACTCCTGAGGTCACCTGCGTG |
| | | | GTCGTGGACGTGTCTCACGAGGACCCCGAAGTCAAGTTCAACT |
| | | | GGTACGTGGATGGCGTCGAAGTGCATAATGCCAAGACCAAACC |
| | | | CCGGGAGGAACAGTACAACTCTACCTATAGAGTCGTGAGTGTC |
| | | | CTGACAGTGCTGCACCAGGACTGGCTGAATGGGAAGGAGTATA |
| | | | AGTGTAAAGTGAGCAACAAAGCCCTGCCCGCCCCAATCGAAAA |
| | | | AACAATCTCTAAAGCAAAAGGACAGCCTCGCGAACCACAGGTG |
| | | | TACACTCTGCCTCCATCTCGGGACGAGCTGACTAAGAACCAGG |
| | | | TCAGTCTGACCTGTCTGGTGAAAGGATTCTATCCCAGCGATATC |
| | | | GCTGTGGAGTGGGAATCCAATGGCCAGCCTGAGAACAATTACA |
| | | | AGACCACACCCCCTGTGCTGGACTCTGATGGCAGTTTCTTTCTG |
| | | | TATAGTAAGCTGACCGTCGATAAATCACGATGGCAGCAGGGGA |
| | | | ACGTGTTCAGCTGTTCAGTGATGCACGAAGCCCTGCACAACCAT |
| | | | TACACCCAGAAGAGCCTGAGCCTGTCTCCCGGC |
| CL_#27287 | VH | 27 | EVQLVQSGAEVKKPGSSVKVSCKASGYTFTDYEMHWVRQAPGQ |
| | (amino | | GLEWIGAIDPETGDTAYNQKFKGRATLTADKSTSTAYMELSSLRS |
| | acid) | | EDTAVYYCTRYFSFAYWGQGTLVTVSS |
| CL_#27287 | VH | 46 | GAGGTGCAGCTGGTGCAGAGCGGAGCCGAGGTGAAGAAGCCC |
| | (DNA) | | GGCAGCTCCGTGAAGGTGTCTTGCAAGGCCAGCGGCTACACCT |
| | | | TCACAGACTATGAGATGCACTGGGTGCGGCAGGCCCCCGGACA |
| | | | GGGACTGGAGTGGATCGGAGCCATCGACCCTGAGACCGGCGAT |
| | | | ACAGCCTACAACCAGAAGTTTAAGGGCAGGGCCACCCTGACAG |
| | | | CCGACAAGAGCACCTCCACAGCCTATATGGAGCTGTCTAGCCT |
| | | | GCGGTCCGAGGATACCGCCGTGTACTATTGTACAAGATACTTCT |
| | | | CTTTTGCCTATTGGGGCCAGGGCACCCTGGTGACAGTGTCCTCT |
| CL_#26960 | LC full | 47 | DVQMTQSPSSVSASVGDRVTITCRSSQSLRHSNGNTYLQWYQQKP |
| | length | | GKAPKLLIYKVSNRFSGVPSRFSGSGSGTDFTLTISSLQPEDFATYF |
| | (amino | | CYQSKHVPYTFGQGTKVEIKRTVAAPSVFIFPPSDEQLKSGTASVV |
| | acid) | | CLLNNFYPREAKVQWKVDNALQSGNSQESVTEQDSKDSTYSLSST |
| | | | LTLSKADYEKHKVYACEVTHQGLSSPVTKSFNRGEC |
| CL_#26960 | LC full | 48 | GACGTGCAGATGACACAGTCCCCAAGCTCCGTGAGCGCCTCCG |
| | length | | TGGGCGATCGGGTGACCATCACATGCAGATCTAGCCAGTCTCT |
| | (DNA) | | GAGGCACAGCAACGGCAATACCTACCTGCAGTGGTATCAGCAG |
| | | | AAGCCCGGCAAGGCCCCTAAGCTGCTGATCTACAAGGTGTCCA |
| | | | ACCGCTTCTCTGGCGTGCCTAGCCGGTTTTCTGGCAGCGGCTCC |
| | | | GGCACCGACTTCACCCTGACAATCTCCTCTCTGCAGCCTGAGGA |
| | | | TTTCGCCACATACTTTTGTTATCAGTCTAAGCACGTGCCATATA |
| | | | CCTTTGGCCAGGGCACAAAGGTGGAGATCAAGAGAACAGTGGC |
| | | | GGCGCCCAGTGTCTTCATTTTTCCCCCTAGCGACGAACAGCTGA |
| | | | AGTCTGGGACAGCCAGTGTGGTCTGTCTGCTGAACAACTTCTAC |
| | | | CCTAGAGAGGCTAAAGTGCAGTGGAAGGTCGATAACGCACTGC |
| | | | AGTCCGGAAATTCTCAGGAGAGTGTGACTGAACAGGACTCAAA |
| | | | AGATAGCACCTATTCCCTGTCAAGCACACTGACTCTGAGCAAG |
| | | | GCCGACTACGAGAAGCATAAAGTGTATGCTTGTGAAGTCACCC |
| | | | ACCAGGGGCTGAGTTCACCAGTCACAAAATCATTCAACAGAGG |
| | | | GGAGTGC |
| CL_#26960 | VL | 28 | DVQMTQSPSSVSASVGDRVTITCRSSQSLRHSNGNTYLQWYQQKP |
| | (amino | | GKAPKLLIYKVSNRFSGVPSRFSGSGSGTDFTLTISSLQPEDFATYF |
| | acid) | | CYQSKHVPYTFGQGTKVEIK |
| CL_#26960 | VL | 49 | GACGTGCAGATGACACAGTCCCCAAGCTCCGTGAGCGCCTCCG |
| | (DNA) | | TGGGCGATCGGGTGACCATCACATGCAGATCTAGCCAGTCTCT |
| | | | GAGGCACAGCAACGGCAATACCTACCTGCAGTGGTATCAGCAG |
| | | | AAGCCCGGCAAGGCCCCTAAGCTGCTGATCTACAAGGTGTCCA |
| | | | ACCGCTTCTCTGGCGTGCCTAGCCGGTTTTCTGGCAGCGGCTCC |
| | | | GGCACCGACTTCACCCTGACAATCTCCTCTCTGCAGCCTGAGGA |
| | | | TTTCGCCACATACTTTTGTTATCAGTCTAAGCACGTGCCATATA |
| | | | CCTTTGGCCAGGGCACAAAGGTGGAGATCAAG |
| CL_#28294 | HC full | 50 | QVQLVQSGAEVKKPGSSVKVSCKASGYTFTDYEMHWVKQAPGH |
| | length | | GLEWIGAIDPETGDTAYNQKFKGKATLTADTSTSTAYMELSSLRSE |
| | (amino | | DTAVYYCTRYFSFAYWGQGTLVTVSSASTKGPSVFPLAPSSKSTSG |
| | acid) | | GTAALGCLVKDYFPEPVTVSWNSGALTSGVHTFPAVLQSSGLYSL |
| | | | SSVVTVPSSSLGTQTYICNVNHKPSNTKVDKKVEPKSCDKTHTCPP |
| | | | CPAPELLGGPSVFLFPPKPKDTLMISRTPEVTCVVVDVSHEDPEVK |
| | | | FNWYVDGVEVHNAKTKPREEQYNSTYRVVSVLTVLHQDWLNGK |
| | | | EYKCKVSNKALPAPIEKTISKAKGQPREPQVYTLPPSRDELTKNQV |
| | | | SLTCLVKGFYPSDIAVEWESNGQPENNYKTTPPVLDSDGSFFLYSK |
| | | | LTVDKSRWQQGNVFSCSVMHEALHNHYTQKSLSLSPG |
| CL_#28294 | HC full | 51 | CAGGTGCAGCTGGTGCAGAGCGGAGCCGAGGTGAAGAAGCCC |
| | length | | GGCAGCTCCGTGAAGGTGTCTTGCAAGGCCAGCGGCTACACCT |
| | (DNA) | | TCACAGACTATGAGATGCACTGGGTGAAGCAGGCCCCCGGACA |
| | | | CGGACTGGAGTGGATCGGAGCCATCGACCCTGAGACAGGCGAT |
| | | | ACCGCCTACAACCAGAAGTTTAAGGGCAAGGCCACCCTGACAG |
| | | | CCGACACCAGCACATCCACCGCCTATATGGAGCTGTCTAGCCTG |
| | | | CGGTCCGAGGATACAGCCGTGTACTATTGTACCAGATACTTCTC |
| | | | TTTTGCCTATTGGGGCCAGGGCACACTGGTGACCGTGTCCTCTG |
| | | | CTAGCACTAAGGGGCCTTCCGTGTTTCCACTGGCTCCCTCTAGT |
| | | | AAATCCACCTCTGGAGGCACAGCTGCACTGGGATGTCTGGTGA |
| | | | AGGATTACTTCCCTGAACCAGTCACAGTGAGTTGGAACTCAGG |
| | | | GGCTCTGACAAGTGGAGTCCATACTTTTCCCGCAGTGCTGCAGT |
| | | | CAAGCGGACTGTACTCCCTGTCCTCTGTGGTCACCGTGCCTAGT |
| | | | TCAAGCCTGGGCACCCAGACATATATCTGCAACGTGAATCACA |
| | | | AGCCATCAAATACAAAAGTCGACAAGAAAGTGGAGCCCAAGA |
| | | | GCTGTGATAAAACTCATACCTGCCCACCTTGTCCGGCGCCAGAA |
| | | | CTGCTGGGAGGACCAAGCGTGTTCCTGTTTCCACCCAAGCCTAA |
| | | | AGACACCCTGATGATTTCCCGGACTCCTGAGGTCACCTGCGTGG |
| | | | TCGTGGACGTGTCTCACGAGGACCCCGAAGTCAAGTTCAACTG |
| | | | GTACGTGGATGGCGTCGAAGTGCATAATGCCAAGACCAAACCC |
| | | | CGGGAGGAACAGTACAACTCTACCTATAGAGTCGTGAGTGTCC |
| | | | TGACAGTGCTGCACCAGGACTGGCTGAATGGGAAGGAGTATAA |
| | | | GTGTAAAGTGAGCAACAAAGCCCTGCCCGCCCCAATCGAAAAA |
| | | | ACAATCTCTAAAGCAAAAGGACAGCCTCGCGAACCACAGGTGT |
| | | | ACACTCTGCCTCCATCTCGGGACGAGCTGACTAAGAACCAGGT |
| | | | CAGTCTGACCTGTCTGGTGAAAGGATTCTATCCCAGCGATATCG |
| | | | CTGTGGAGTGGGAATCCAATGGCCAGCCTGAGAACAATTACAA |
| | | | GACCACACCCCCTGTGCTGGACTCTGATGGCAGTTTCTTTCTGT |
| | | | ATAGTAAGCTGACCGTCGATAAATCACGATGGCAGCAGGGGAA |
| | | | CGTGTTCAGCTGTTCAGTGATGCACGAAGCCCTGCACAACCATT |
| | | | ACACCCAGAAGAGCCTGAGCCTGTCTCCCGGC |
| CL_#28294 | VH | 29 | QVQLVQSGAEVKKPGSSVKVSCKASGYTFTDYEMHWVKQAPGH |
| | (amino | | GLEWIGAIDPETGDTAYNQKFKGKATLTADTSTSTAYMELSSLRSE |
| | acid) | | DTAVYYCTRYFSFAYWGQGTLVTVSS |
| CL_#28294 | VH | 52 | CAGGTGCAGCTGGTGCAGAGCGGAGCCGAGGTGAAGAAGCCC |
| | (DNA) | | GGCAGCTCCGTGAAGGTGTCTTGCAAGGCCAGCGGCTACACCT |
| | | | TCACAGACTATGAGATGCACTGGGTGAAGCAGGCCCCCGGACA |
| | | | CGGACTGGAGTGGATCGGAGCCATCGACCCTGAGACAGGCGAT |
| | | | ACCGCCTACAACCAGAAGTTTAAGGGCAAGGCCACCCTGACAG |
| | | | CCGACACCAGCACATCCACCGCCTATATGGAGCTGTCTAGCCTG |
| | | | CGGTCCGAGGATACAGCCGTGTACTATTGTACCAGATACTTCTC |
| | | | TTTTGCCTATTGGGGCCAGGGCACACTGGTGACCGTGTCCTCT |
| CL_#28295 | LC full | 53 | EIVLTQSPATLSLSPGERATISCRSSQSLRHSNGNTYLQWYQQKPG |
| | length | | QAPRLLIYKVSNRFSGVPARFSGSGSGTDFTLTISSLEPEDFAVYFC |
| | (amino | | YQSKHVPYTFGGGTKVEIKRTVAAPSVFIFPPSDEQLKSGTASVVC |
| | acid) | | LLNNFYPREAKVQWKVDNALQSGNSQESVTEQDSKDSTYSLSSTL |
| | | | TLSKADYEKHKVYACEVTHQGLSSPVTKSFNRGEC |
| CL_#28295 | LC full | 54 | GAGATCGTGCTGACCCAGTCCCCAGCCACACTGTCCCTGTCTCC |
| | length | | AGGAGAGAGGGCAACCATCTCTTGCAGAAGCTCCCAGAGCCTG |
| | (DNA) | | AGGCACTCCAACGGCAATACATACCTGCAGTGGTATCAGCAGA |
| | | | AGCCAGGACAGGCCCCTCGCCTGCTGATCTACAAGGTGTCTAA |
| | | | CCGCTTCAGCGGCGTGCCCGCCCGGTTTAGCGGCTCCGGCTCTG |
| | | | GCACCGACTTCACCCTGACAATCTCTAGCCTGGAGCCTGAGGAT |
| | | | TTCGCCGTGTACTTTTGTTATCAGTCCAAGCACGTGCCATATAC |
| | | | CTTTGGCGGCGGCACAAAGGTGGAGATCAAGCGGACAGTGGCG |
| | | | GCGCCCAGTGTCTTCATTTTTCCCCCTAGCGACGAACAGCTGAA |
| | | | GTCTGGGACAGCCAGTGTGGTCTGTCTGCTGAACAACTTCTACC |
| | | | CTAGAGAGGCTAAAGTGCAGTGGAAGGTCGATAACGCACTGCA |
| | | | GTCCGGAAATTCTCAGGAGAGTGTGACTGAACAGGACTCAAAA |
| | | | GATAGCACCTATTCCCTGTCAAGCACACTGACTCTGAGCAAGG |
| | | | CCGACTACGAGAAGCATAAAGTGTATGCTTGTGAAGTCACCCA |
| | | | CCAGGGGCTGAGTTCACCAGTCACAAAATCATTCAACAGAGGG |
| | | | GAGTGC |
| CL_#28295 | VL | 30 | EIVLTQSPATLSLSPGERATISCRSSQSLRHSNGNTYLQWYQQKPG |
| | (amino | | QAPRLLIYKVSNRFSGVPARFSGSGSGTDFTLTISSLEPEDFAVYFC |
| | acid) | | YQSKHVPYTFGGGTKVEIK |
| CL_#28295 | VL | 55 | GAGATCGTGCTGACCCAGTCCCCAGCCACACTGTCCCTGTCTCC |
| | (DNA) | | AGGAGAGAGGGCAACCATCTCTTGCAGAAGCTCCCAGAGCCTG |
| | | | AGGCACTCCAACGGCAATACATACCTGCAGTGGTATCAGCAGA |
| | | | AGCCAGGACAGGCCCCTCGCCTGCTGATCTACAAGGTGTCTAA |
| | | | CCGCTTCAGCGGCGTGCCCGCCCGGTTTAGCGGCTCCGGCTCTG |
| | | | GCACCGACTTCACCCTGACAATCTCTAGCCTGGAGCCTGAGGAT |
| | | | TTCGCCGTGTACTTTTGTTATCAGTCCAAGCACGTGCCATATAC |
| | | | CTTTGGCGGCGGCACAAAGGTGGAGATCAAG |
| CL_#29372 | HC full | 56 | QVQLVQSGAEVKKPGSSVKVSCKASGYTFTDYEMHWVKQAPGH |
| | length | | GLEWIGAIDPETGDTAYNQKFKGKATLTADTSTSTAYMELSSLRSE |
| | (amino | | DTAVYYCTRYFSFAYWGQGTLVTVSSASTKGPSVFPLAPSSKSTSG |
| | acid) | | GTAALGCLVKDYFPEPVTVSWNSGALTSGVHTFPAVLQSSGLYSL |
| | | | SSVVTVPSSSLGTQTYICNVNHKPSNTKVDKKVEPKSCDKTHTCPP |
| | | | CPAPELLGGPSVFLFPPKPKDTLMISRTPEVTCVVVDVSHEDPEVK |
| | | | FNWYVDGVEVHNAKTKPREEQYNSTYRVVSVLTVLHQDWLNGK |
| | | | EYKCKVSNKALPAPIEKTISKAKGQPREPQVYTLPPSRDELTKNQV |
| | | | SLTCLVKGFYPSDIAVEWESNGQPENNYKTTPPVLDSDGSFFLYSK |
| | | | LTVDKSRWQQGNVFSCSVMHEALHNHYTQKSLSLSPGK |
| CL_#29372 | HC full | 57 | CAGGTCCAGCTTGTACAATCCGGGGCAGAGGTCAAGAAGCCAG |
| | length | | GCAGCAGCGTTAAAGTGTCCTGCAAGGCTTCTGGATACACATTT |
| | (DNA) | | ACCGATTATGAGATGCACTGGGTCAAGCAGGCTCCCGGGCATG |
| | | | GTCTCGAGTGGATTGGCGCCATCGACCCTGAAACAGGAGATAC |
| | | | CGCCTATAACCAGAAGTTTAAAGGGAAGGCCACCCTGACCGCT |
| | | | GATACATCAACATCCACCGCCTACATGGAGCTCAGTTCTTTGCG |
| | | | GAGCGAGGACACTGCCGTGTACTACTGTACCCGGTACTTTAGCT |
| | | | TCGCCTATTGGGGCCAGGGCACACTGGTGACAGTTTCTTCCGCT |
| | | | AGCACCAAGGGCCCATCGGTCTTCCCCCTGGCACCCTCCTCCAA |
| | | | GAGCACCTCTGGGGGCACAGCGGCCCTGGGCTGCCTGGTCAAG |
| | | | GACTACTTCCCCGAACCGGTGACGGTGTCGTGGAACTCAGGCG |
| | | | CCCTGACCAGCGGCGTGCACACCTTCCCGGCCGTCCTACAGTCC |
| | | | TCAGGACTCTACTCCCTCAGCAGCGTGGTGACCGTGCCCTCCAG |
| | | | CAGCTTGGGCACCCAGACCTACATCTGCAACGTGAATCACAAG |
| | | | CCCAGCAACACCAAGGTGGACAAGAAGGTTGAGCCCAAATCTT |
| | | | GTGACAAAACTCACACATGCCCACCGTGCCCAGCACCTGAACT |
| | | | CCTGGGGGGACCGTCAGTCTTCCTCTTCCCCCCAAAACCCAAGG |
| | | | ACACCCTCATGATCTCCCGGACCCCTGAGGTCACATGCGTGGTG |
| | | | GTGGACGTGAGCCACGAAGACCCTGAGGTCAAGTTCAACTGGT |
| | | | ACGTGGACGGCGTGGAGGTGCATAATGCCAAGACAAAGCCGCG |
| | | | GGAGGAGCAGTACAACAGCACGTACCGTGTGGTCAGCGTCCTC |
| | | | ACCGTCCTGCACCAGGACTGGCTGAATGGCAAGGAGTACAAGT |
| | | | GCAAGGTCTCCAACAAAGCCCTCCCAGCCCCCATCGAGAAAAC |
| | | | CATCTCCAAAGCCAAAGGGCAGCCCCGAGAACCACAGGTGTAC |
| | | | ACCCTGCCCCCATCCCGGGACGAGCTGACCAAGAACCAGGTCA |
| | | | GCCTGACCTGCCTGGTCAAAGGCTTCTATCCCAGCGACATCGCC |
| | | | GTGGAGTGGGAGAGCAATGGGCAGCCGGAGAACAACTACAAG |
| | | | ACCACGCCTCCCGTGCTGGACTCCGACGGCTCCTTCTTCCTCTA |
| | | | CAGCAAGCTCACCGTGGACAAGAGCAGGTGGCAGCAGGGGAA |
| | | | CGTCTTCTCATGCTCCGTGATGCATGAGGCTCTGCACAACCACT |
| | | | ACACGCAGAAGAGCCTCTCCCTGTCTCCGGGCAAA |
| CL_#30542 | LC full | 58 | EIVLTQSPATLSLSPGERATISCRSSQSLRHSNQNTYLQWYQQKPG |
| | length | | QAPRLLIYKVSNRFSGVPARFSGSGSGTDFTLTISSLEPEDFAVYFC |
| | (amino | | YQSKHVPYTFGGGTKVEIKRTVAAPSVFIFPPSDEQLKSGTASVVC |
| | acid) | | LLNNFYPREAKVQWKVDNALQSGNSQESVTEQDSKDSTYSLSSTL |
| | | | TLSKADYEKHKVYACEVTHQGLSSPVTKSFNRGEC |
| CL_#30542 | LC full | 59 | GAGATCGTGCTGACCCAGTCCCCAGCCACACTGAGCCTGTCCCC |
| | length | | AGGAGAGAGGGCAACCATCTCCTGCAGAAGCTCCCAGTCTCTG |
| | (DNA) | | AGGCACTCTAACCAGAACACATACCTGCAGTGGTATCAGCAGA |
| | | | AGCCAGGACAGGCCCCTCGCCTGCTGATCTACAAGGTGTCTAA |
| | | | TCGCTTCAGCGGCGTGCCCGCCCGGTTTTCTGGCAGCGGCTCCG |
| | | | GCACCGACTTCACCCTGACAATCTCCTCTCTGGAGCCTGAGGAT |
| | | | TTCGCCGTGTACTTTTGTTATCAGAGCAAGCACGTGCCATATAC |
| | | | CTTTGGCGGCGGCACAAAGGTGGAGATCAAGCGGACAGTGGCG |
| | | | GCGCCCAGTGTCTTCATTTTTCCCCCTAGCGACGAACAGCTGAA |
| | | | GTCTGGGACAGCCAGTGTGGTCTGTCTGCTGAACAACTTCTACC |
| | | | CTAGAGAGGCTAAAGTGCAGTGGAAGGTCGATAACGCACTGCA |
| | | | GTCCGGAAATTCTCAGGAGAGTGTGACTGAACAGGACTCAAAA |
| | | | GATAGCACCTATTCCCTGTCAAGCACACTGACTCTGAGCAAGG |
| | | | CCGACTACGAGAAGCATAAAGTGTATGCTTGTGAAGTCACCCA |
| | | | CCAGGGGCTGAGTTCACCAGTCACAAAATCATTCAACAGAGGG |
| | | | GAGTGC |
| CL_#30542 | VL | 60 | EIVLTQSPATLSLSPGERATISCRSSQSLRHSNQNTYLQWYQQKPG |
| | (amino | | QAPRLLIYKVSNRFSGVPARFSGSGSGTDFTLTISSLEPEDFAVYFC |
| | acid) | | YQSKHVPYTFGGGTKVEIK |
| CL_#30542 | VL | 61 | GAGATCGTGCTGACCCAGTCCCCAGCCACACTGAGCCTGTCCCC |
| | (DNA) | | AGGAGAGAGGGCAACCATCTCCTGCAGAAGCTCCCAGTCTCTG |
| | | | AGGCACTCTAACCAGAACACATACCTGCAGTGGTATCAGCAGA |
| | | | AGCCAGGACAGGCCCCTCGCCTGCTGATCTACAAGGTGTCTAA |
| | | | TCGCTTCAGCGGCGTGCCCGCCCGGTTTTCTGGCAGCGGCTCCG |
| | | | GCACCGACTTCACCCTGACAATCTCCTCTCTGGAGCCTGAGGAT |
| | | | TTCGCCGTGTACTTTTGTTATCAGAGCAAGCACGTGCCATATAC |
| | | | CTTTGGCGGCGGCACAAAGGTGGAGATCAAG |
| CL_#30544 | LC full | 62 | EIVLTQSPATLSLSPGERATISCRSSQSLRHSNKNTYLQWYQQKPG |
| | length | | QAPRLLIYKVSNRFSGVPARFSGSGSGTDFTLTISSLEPEDFAVYFC |
| | (amino | | YQSKHVPYTFGGGTKVEIKRTVAAPSVFIFPPSDEQLKSGTASVVC |
| | acid) | | LLNNFYPREAKVQWKVDNALQSGNSQESVTEQDSKDSTYSLSSTL |
| | | | TLSKADYEKHKVYACEVTHQGLSSPVTKSFNRGEC |
| CL_#30544 | LC full | 63 | GAGATCGTGCTGACCCAGTCCCCAGCCACACTGAGCCTGTCCCC |
| | length | | AGGAGAGAGGGCAACCATCTCCTGCAGAAGCTCCCAGTCTCTG |
| | (DNA) | | AGGCACTCTAACAAGAACACATACCTGCAGTGGTATCAGCAGA |
| | | | AGCCAGGACAGGCCCCTCGCCTGCTGATCTACAAGGTGTCTAA |
| | | | TCGCTTCAGCGGCGTGCCCGCCCGGTTTTCTGGCAGCGGCTCCG |
| | | | GCACCGACTTCACCCTGACAATCTCCTCTCTGGAGCCTGAGGAT |
| | | | TTCGCCGTGTACTTTTGTTATCAGAGCAAGCACGTGCCATATAC |
| | | | CTTTGGCGGCGGCACAAAGGTGGAGATCAAGCGGACAGTGGCG |
| | | | GCGCCCAGTGTCTTCATTTTTCCCCCTAGCGACGAACAGCTGAA |
| | | | GTCTGGGACAGCCAGTGTGGTCTGTCTGCTGAACAACTTCTACC |
| | | | CTAGAGAGGCTAAAGTGCAGTGGAAGGTCGATAACGCACTGCA |
| | | | GTCCGGAAATTCTCAGGAGAGTGTGACTGAACAGGACTCAAAA |
| | | | GATAGCACCTATTCCCTGTCAAGCACACTGACTCTGAGCAAGG |
| | | | CCGACTACGAGAAGCATAAAGTGTATGCTTGTGAAGTCACCCA |
| | | | CCAGGGGCTGAGTTCACCAGTCACAAAATCATTCAACAGAGGG |
| | | | GAGTGC |
| CL_#30544 | VL | 64 | EIVLTQSPATLSLSPGERATISCRSSQSLRHSNKNTYLQWYQQKPG |
| | (amino | | QAPRLLIYKVSNRFSGVPARFSGSGSGTDFTLTISSLEPEDFAVYFC |
| | acid) | | YQSKHVPYTFGGGTKVEIK |
| CL_#30544 | VL | 65 | GAGATCGTGCTGACCCAGTCCCCAGCCACACTGAGCCTGTCCCC |
| | (DNA) | | AGGAGAGAGGGCAACCATCTCCTGCAGAAGCTCCCAGTCTCTG |
| | | | AGGCACTCTAACAAGAACACATACCTGCAGTGGTATCAGCAGA |
| | | | AGCCAGGACAGGCCCCTCGCCTGCTGATCTACAAGGTGTCTAA |
| | | | TCGCTTCAGCGGCGTGCCCGCCCGGTTTTCTGGCAGCGGCTCCG |
| | | | GCACCGACTTCACCCTGACAATCTCCTCTCTGGAGCCTGAGGAT |
| | | | TTCGCCGTGTACTTTTGTTATCAGAGCAAGCACGTGCCATATAC |
| | | | CTTTGGCGGCGGCACAAAGGTGGAGATCAAG |
| CL_#30545 | LC full | 66 | EIVLTQSPATLSLSPGERATISCRSSQSLRHSNRNTYLQWYQQKPG |
| | length | | QAPRLLIYKVSNRFSGVPARFSGSGSGTDFTLTISSLEPEDFAVYFC |
| | (amino | | YQSKHVPYTFGGGTKVEIKRTVAAPSVFIFPPSDEQLKSGTASVVC |
| | acid) | | LLNNFYPREAKVQWKVDNALQSGNSQESVTEQDSKDSTYSLSSTL |
| | | | TLSKADYEKHKVYACEVTHQGLSSPVTKSFNRGEC |
| CL_#30545 | LC full | 67 | GAGATCGTGCTGACCCAGTCCCCAGCCACACTGAGCCTGTCCCC |
| | length | | AGGAGAGAGGGCAACCATCTCCTGCAGAAGCTCCCAGTCTCTG |
| | (DNA) | | AGGCACTCTAACAGAAACACATACCTGCAGTGGTATCAGCAGA |
| | | | AGCCAGGACAGGCCCCTCGCCTGCTGATCTACAAGGTGTCTAA |
| | | | TCGCTTCAGCGGCGTGCCCGCCCGGTTTTCTGGCAGCGGCTCCG |
| | | | GCACCGACTTCACCCTGACAATCTCCTCTCTGGAGCCTGAGGAT |
| | | | TTCGCCGTGTACTTTTGTTATCAGAGCAAGCACGTGCCATATAC |
| | | | CTTTGGCGGCGGCACAAAGGTGGAGATCAAGCGGACAGTGGCG |
| | | | GCGCCCAGTGTCTTCATTTTTCCCCCTAGCGACGAACAGCTGAA |
| | | | GTCTGGGACAGCCAGTGTGGTCTGTCTGCTGAACAACTTCTACC |
| | | | CTAGAGAGGCTAAAGTGCAGTGGAAGGTCGATAACGCACTGCA |
| | | | GTCCGGAAATTCTCAGGAGAGTGTGACTGAACAGGACTCAAAA |
| | | | GATAGCACCTATTCCCTGTCAAGCACACTGACTCTGAGCAAGG |
| | | | CCGACTACGAGAAGCATAAAGTGTATGCTTGTGAAGTCACCCA |
| | | | CCAGGGGCTGAGTTCACCAGTCACAAAATCATTCAACAGAGGG |
| | | | GAGTGC |
| CL_#30545 | VL | 68 | EIVLTQSPATLSLSPGERATISCRSSQSLRHSNRNTYLQWYQQKPG |
| | (amino | | QAPRLLIYKVSNRFSGVPARFSGSGSGTDFTLTISSLEPEDFAVYFC |
| | acid) | | YQSKHVPYTFGGGTKVEIK |
| CL_#30545 | VL | 69 | GAGATCGTGCTGACCCAGTCCCCAGCCACACTGAGCCTGTCCCC |
| | (DNA) | | AGGAGAGAGGGCAACCATCTCCTGCAGAAGCTCCCAGTCTCTG |
| | | | AGGCACTCTAACAGAAACACATACCTGCAGTGGTATCAGCAGA |
| | | | AGCCAGGACAGGCCCCTCGCCTGCTGATCTACAAGGTGTCTAA |
| | | | TCGCTTCAGCGGCGTGCCCGCCCGGTTTTCTGGCAGCGGCTCCG |
| | | | GCACCGACTTCACCCTGACAATCTCCTCTCTGGAGCCTGAGGAT |
| | | | TTCGCCGTGTACTTTTGTTATCAGAGCAAGCACGTGCCATATAC |
| | | | CTTTGGCGGCGGCACAAAGGTGGAGATCAAG |

1-59. (canceled)

60. An antibody-drug conjugate having the structure: 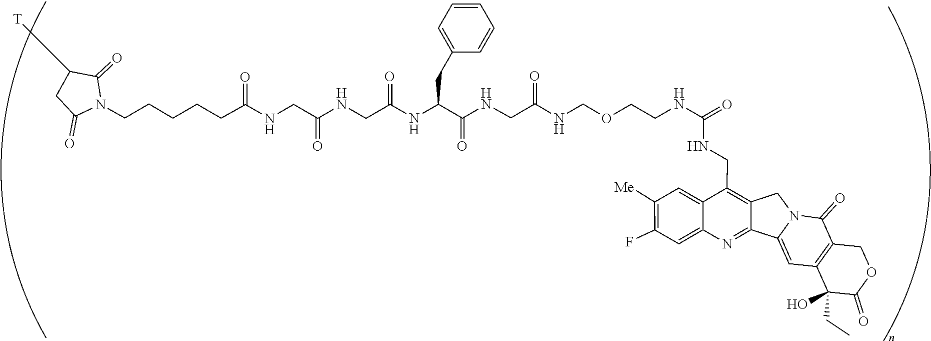
wherein:
n is between about 4 and about 8, and
T is an anti-glypican-3 (GPC3) antibody construct comprising an antigen-binding domain operably linked to an IgG Fc region, wherein the antigen-binding domain binds to human GPC3 and comprises:
a) the heavy chain CDR sequences (HCDR1, HCDR2, HCDR3) of the heavy chain variable (VH) domain having a sequence as set forth in SEQ ID NO: 29, and
b) the light chain CDR sequences (LCDR1, LCDR2, LCDR3) of the light chain variable (VL) domain having a sequence as set forth in SEQ ID NO: 30.

61. The antibody-drug conjugate according to claim 60, wherein the antigen-binding domain comprises:
a) a HCDR1 amino acid sequence comprising the sequence as set forth in SEQ ID NO: 6, a HCDR2 amino acid sequence comprising the sequence as set forth in SEQ ID NO: 7, and a HCDR3 amino acid sequence comprising the sequence as set forth in SEQ ID NO: 8, and
b) a LCDR1 amino acid sequence comprising the sequence as set forth in SEQ ID NO: 18, a LCDR2 amino acid sequence comprising the sequence as set forth in SEQ ID NO: 19, and a LCDR3 amino acid sequence comprising the sequence as set forth in SEQ ID NO: 17.

62. The antibody-drug conjugate according to claim 60, wherein the antigen-binding domain comprises a VH domain having at least 90% sequence identity with the sequence as set forth in SEQ ID NO: 29 and a VL domain having at least 90% sequence identity with the sequence as set forth in SEQ ID NO: 30.

63. The antibody-drug conjugate according to claim 60, wherein the antigen-binding domain comprises a VH domain comprising the sequence as set forth in SEQ ID NO: 29 and a VL domain comprising the sequence as set forth in SEQ ID NO: 30.

64. The antibody-drug conjugate according to claim 60, wherein the antigen-binding domain comprises a VH domain consisting of the sequence as set forth in SEQ ID NO: 29 and a VL domain consisting of the sequence as set forth in SEQ ID NO: 30.

65. The antibody-drug conjugate according to claim 60, wherein the IgG Fc region is an IgG1 Fc region.

66. The antibody-drug conjugate according to claim 60, wherein the IgG Fc region is a human IgG1 Fc region.

67. The antibody-drug conjugate according to claim 60, further comprising a second antigen-binding domain operably linked to the IgG Fc region, wherein the second antigen-binding domain binds to human GPC3 and comprises:
a) the heavy chain CDR sequences (HCDR1, HCDR2, HCDR3) of the heavy chain variable (VH) domain having a sequence as set forth in SEQ ID NO: 29, and
b) the light chain CDR sequences (LCDR1, LCDR2, LCDR3) of the light chain variable (VL) domain having a sequence as set forth in SEQ ID NO: 30.

68. The antibody-drug conjugate according to claim 67, wherein the second antigen-binding domain comprises:
a) a HCDR1 amino acid sequence comprising the sequence as set forth in SEQ ID NO: 6, a HCDR2 amino acid sequence comprising the sequence as set forth in SEQ ID NO: 7, and a HCDR3 amino acid sequence comprising the sequence as set forth in SEQ ID NO: 8, and
b) a LCDR1 amino acid sequence comprising the sequence as set forth in SEQ ID NO: 18, a LCDR2 amino acid sequence comprising the sequence as set forth in SEQ ID NO: 19, and a LCDR3 amino acid sequence comprising the sequence as set forth in SEQ ID NO: 17.

69. The antibody-drug conjugate according to claim 67, wherein the antigen-binding domain comprises a VH domain having at least 90% sequence identity with the sequence as set forth in SEQ ID NO: 29 and a VL domain having at least 90% sequence identity with the sequence as set forth in SEQ ID NO: 30.

70. The antibody-drug conjugate according to claim 67, wherein the antigen-binding domain comprises a VH domain comprising the sequence as set forth in SEQ ID NO: 29 and a VL domain comprising the sequence as set forth in SEQ ID NO: 30.

71. The antibody-drug conjugate according to claim 67, wherein the antigen-binding domain comprises a VH domain consisting of the sequence as set forth in SEQ ID NO: 29 and a VL domain consisting of the sequence as set forth in SEQ ID NO: 30.

72. The antibody-drug conjugate according to claim 67, wherein the IgG Fc region is an IgG1 Fc region.

73. The antibody-drug conjugate according to claim 67, wherein the IgG Fc region is a human IgG1 Fc region.

74. The antibody-drug conjugate according to claim 67, wherein the anti-GPC3 antigen-binding construct comprises:
a) two heavy chains, each comprising the sequence as set forth in SEQ ID NO: 37, and two light chains, each comprising the sequence as set forth in SEQ ID NO: 38, or
b) two heavy chains, each comprising the sequence as set forth in SEQ ID NO: 56, and two light chains, each comprising the sequence as set forth in SEQ ID NO: 38.

75. The antibody-drug conjugate according to claim 60, wherein n is about 4.

76. The antibody-drug conjugate according to claim 67, wherein n is about 4.

77. The antibody-drug conjugate according to claim 74, wherein n is about 4.

78. An antibody-drug conjugate having the structure: 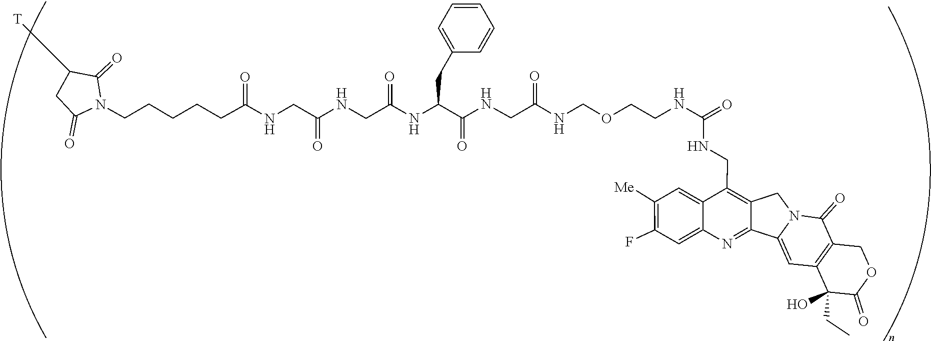
wherein:
n is about 4, and
T is an anti-glypican-3 (GPC3) antibody construct comprising two antigen-binding domains operably linked to an IgG Fc region, wherein each antigen-binding domain binds to human GPC3 and comprises a heavy chain variable (VH) domain comprising the sequence as set forth in SEQ ID NO: 29, and a light chain variable (VL) domain comprising the sequence as set forth in SEQ ID NO: 30.

79. The antibody-drug conjugate according to claim 78, wherein the VH domain consists of the sequence as set forth in SEQ ID NO: 29 and the VL domain consists of the sequence as set forth in SEQ ID NO: 30.

80. The antibody-drug conjugate according to claim 78, wherein the IgG Fc region is an IgG1 Fc region.

81. The antibody-drug conjugate according to claim 78, wherein the IgG Fc region is a human IgG1 Fc region.

82. The antibody-drug conjugate according to claim 78, wherein the anti-GPC3 antigen-binding construct comprises:
a) two heavy chains, each comprising the sequence as set forth in SEQ ID NO: 37, and two light chains, each comprising the sequence as set forth in SEQ ID NO: 38, or
b) two heavy chains, each comprising the sequence as set forth in SEQ ID NO: 56, and two light chains, each comprising the sequence as set forth in SEQ ID NO: 38.

83. A pharmaceutical composition comprising the antibody-drug conjugate according to claim 60.

84. A pharmaceutical composition comprising the antibody-drug conjugate according to claim 78.

85. A method of killing cancer cells comprising contacting the cells with the antibody-drug conjugate according to claim 60.

86. A method of treating cancer in a subject comprising administering to the subject the antibody-drug conjugate according to claim 60.

87. A method of treating cancer in a subject comprising administering to the subject an antibody-drug conjugate having Formula (X):
T-[L-(D)ₘ]ₙ (X)
wherein:
m is an integer between 1 and 4;
n is an integer between 1 and 10;
T is an anti-GPC3 (glypican-3) antibody construct, comprising an antigen-binding domain that binds to human GPC3, the antigen-binding domain comprising:
a) a heavy chain CDR1 (HCDR1) amino acid sequence comprising the sequence as set forth in SEQ ID NO: 6, a heavy chain CDR2 (HCDR2) amino acid sequence comprising the sequence as set forth in SEQ ID NO: 7, and a heavy chain CDR3 (HCDR3) amino acid sequence comprising the sequence as set forth in SEQ ID NO: 8, and
b) a light chain CDR1 (LCDR1) amino acid sequence comprising the sequence as set forth in SEQ ID NO: 18, a light chain CDR2 (LCDR2) amino acid sequence comprising the sequence as set forth in SEQ ID NO: 19, and a light chain CDR3 (LCDR3) amino acid sequence comprising the sequence as set forth in SEQ ID NO: 17;
L is a linker, and
D is a compound of Formula I: 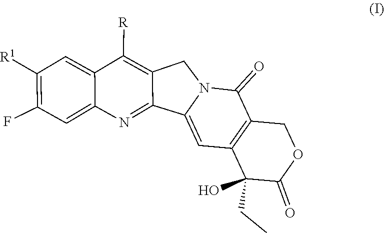
wherein:
R¹ is selected from: —H, —CH₃, —CHF₂, —CF₃, —F, —Br, —Cl, —OH, —OCH₃, —OCF₃ and —NH₂, and
R² is selected from: —H, —CH₃, —CF₃, —F, —Br, —Cl, —OH, —OCH₃ and —OCF₃, and wherein:
when R¹ is —NH₂, then R is R³ or R⁴, and when R¹ is other than —NH₂, then R is R⁴;
R³ is selected from: —H, —C₁-C₆ alkyl, —C₃-C₈ cycloalkyl, —(C₁-C₆ alkyl)-O—R⁵, 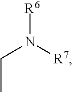
—CO₂R⁸, -aryl, -heteroaryl and —(C₁-C₆ alkyl)-aryl;
R is selected from: 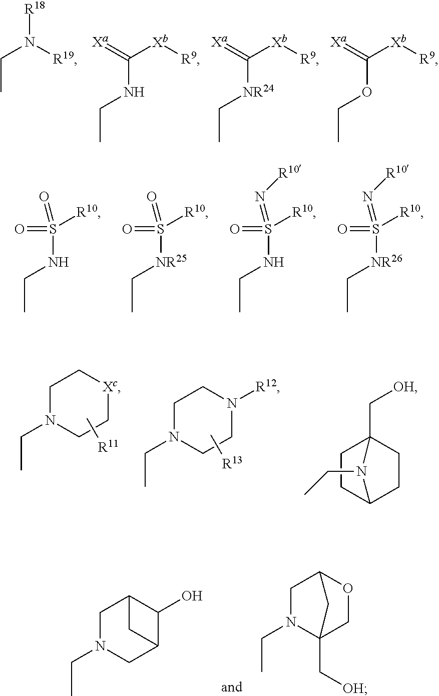
R⁵ is selected from: —H, —C₁-C₆ alkyl, —C₃-C₈ cycloalkyl, -aryl, -heteroaryl and —(C₁-C₆ alkyl)-aryl;
R⁶ and R⁷ are each independently selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, —NR¹⁴R^{14′}, -aryl, -heteroaryl and —(C₁-C₆ alkyl)-aryl;
R^{10′} is selected from: —H, —C₁-C₆ alkyl, —C₃-C₈ cycloalkyl, -aryl, -heteroaryl, and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl, —S(O)₂R¹⁶ and 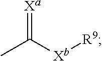
R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁷ is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R¹⁸ and R¹⁹ taken together with the N atom to which they are bonded form a 4-, 5-, 6- or 7-membered ring having 0 to 3 substituents selected from: halogen, —C₁-C₆ alkyl, —C₃-C₈ cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, O and S, and
X^{c} is selected from; O, S and S(O)₂,
with the proviso that the compound is other than (S)-9-amino-11-butyl-4-ethyl-4-hydroxy-1,12-dihydro-14H-pyrano[3′,4′:6,7]indolizino[1,2-b]quinoline-3,14(4H)-dione.

88. A method of preparing an antibody-drug conjugate having Formula (X):
T-[L-(D)ₘ]ₙ (X)
wherein:
m is an integer between 1 and 4;
n is an integer between 1 and 10;
T is an anti-GPC3 (glypican-3) antibody construct, comprising an antigen-binding domain that binds to human GPC3, the antigen-binding domain comprising:
a) a heavy chain CDR1 (HCDR1) amino acid sequence comprising the sequence as set forth in SEQ ID NO: 6, a heavy chain CDR2 (HCDR2) amino acid sequence comprising the sequence as set forth in SEQ ID NO: 7, and a heavy chain CDR3 (HCDR3) amino acid sequence comprising the sequence as set forth in SEQ ID NO: 8, and
b) a light chain CDR1 (LCDR1) amino acid sequence comprising the sequence as set forth in SEQ ID NO: 18, a light chain CDR2 (LCDR2) amino acid sequence comprising the sequence as set forth in SEQ ID NO: 19, and a light chain CDR3 (LCDR3) amino acid sequence comprising the sequence as set forth in SEQ ID NO: 17;
L is a linker, and
D is a compound of Formula I: 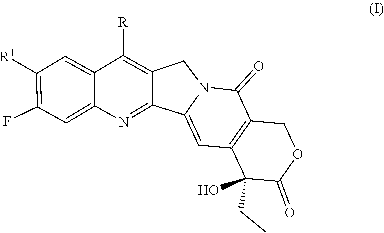
wherein:
R¹ is selected from: —H, —CH₃, —CHF₂, —CF₃, —F, —Br, —Cl, —OH, —OCH₃, —OCF₃ and —NH₂, and
R² is selected from: —H, —CH₃, —CF₃, —F, —Br, —Cl, —OH, —OCH₃ and —OCF₃, and wherein:
when R¹ is —NH₂, then R is R³ or R⁴, and when R¹ is other than —NH₂, then R is R⁴;
R³ is selected from: —H, —C₁-C₆ alkyl, —C₃-C₈ cycloalkyl, —(C₁-C₆ alkyl)-O—R⁵, 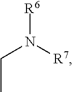
—CO₂R⁸, -aryl, -heteroaryl and —(C₁-C₆ alkyl)-aryl;
R⁴ is selected from: 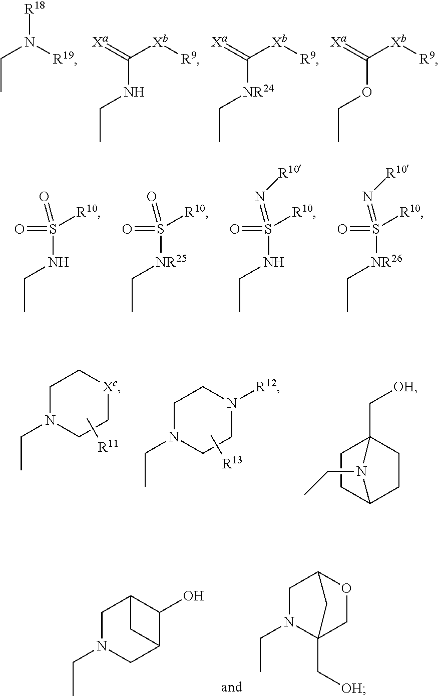
R⁵ is selected from: —H, —C₁-C₆ alkyl, —C₃-C₈ cycloalkyl, -aryl, -heteroaryl and —(C₁-C₆ alkyl)-aryl;
R⁶ and R⁷ are each independently selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, —NR¹⁴R^{14′}, -aryl, -heteroaryl and —(C₁-C₆ alkyl)-aryl;
R^{10′} is selected from: —H, —C₁-C₆ alkyl, —C₃-C₈ cycloalkyl, -aryl, -heteroaryl, and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl, —S(O)₂R¹⁶ and 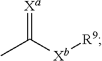
R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁷ is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R¹⁸ and R¹⁹ taken together with the N atom to which they are bonded form a 4-, 5-, 6- or 7-membered ring having 0 to 3 substituents selected from: halogen, —C₁-C₆ alkyl, —C₃-C₈ cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, O and S, and
X^{c} is selected from; O, S and S(O)₂,
with the proviso that the compound is other than (S)-9-amino-11-butyl-4-ethyl-4-hydroxy-1,12-dihydro-14H-pyrano[3′,4′:6,7]indolizino[1,2-b]quinoline-3,14(4H)-dione, the method comprising:
(a) preparing a drug-linker D-(L)ₘ, and
(b) conjugating the drug-linker D-(L)ₘ to T.
